(19) United States
(12) Patent Application Publication
(10) Pub. No.: US 2026/0044078 A1
(43) Pub. Date: Feb. 12, 2026

(54) ONIUM-SALT-TYPE MONOMER, MONOMERIC PHOTO-ACID GENERATOR, POLYMER, CHEMICALLY-AMPLIFIED RESIST COMPOSTION, AND PATTERNING PROCESS

(71) Applicant: SHIN-ETSU CHEMICAL CO., LTD., Tokyo (JP)

(72) Inventor: Masahiro FUKUSHIMA, Joetsu-shi (JP)

(73) Assignee: SHIN-ETSU CHEMICAL CO., LTD., Tokyo (JP)

(21) Appl. No.: 19/290,483

(22) Filed: Aug. 5, 2025

(30) Foreign Application Priority Data
Aug. 6, 2024 (JP) 2024-130228

(51) Int. Cl. G03F 7/004; C07C 381/12; C08G 63/06; G03F 7/00; G03F 7/039

(52) U.S. Cl. CPC G03F 7/0045; C07C 381/12; C08G 63/06; G03F 7/0046; G03F 7/0397; G03F 7/70033

(57) ABSTRACT

An onium-salt-type monomer is represented by the formula (a), where “n1” is 0 or 1, “n2” is 1 to 4, “n3” is 0 to 2, “n4” is 0 or 1, “n5” is 0 to 4, and “n6” is 0 to 2; R^{A} is a hydrogen atom, a fluorine atom, a methyl group, or a trifluoromethyl group; R^{AL} is an acid-labile group; R¹ is a halogen atom, a nitro group, a cyano group; R² is a halogen atom other than a fluorine atom, a nitro group, a cyano group; R^{F} is a fluorine atom, a fluorinated saturated hydrocarbyl group, a fluorinated saturated hydrocarbyloxy group, or a fluorinated saturated hydrocarbylthio group having 1 to 6 carbon atoms; L^{A} and L^{B} are each a single bond, an ester bond; X^{L} is a single bond or a hydrocarbylene group; and Z⁺ is an onium cation.

### TECHNICAL FIELD

The present invention relates to: an onium-salt-type monomer; a monomeric photo-acid generator; a polymer; a chemically-amplified resist composition; and a patterning process.

### BACKGROUND ART

As LSIs advance toward higher integration and higher processing speed, miniaturization of pattern rule is progressing rapidly. In particular, the wide-spreading flash memory market and the demand for increased storage capacities drive forward the miniaturization technology. As the advanced miniaturization technology, manufacturing of 65-nm node devices by the ArF lithography has been implemented in a mass scale, and manufacturing of 45-nm node devices by the next-generation ArF immersion lithography is approaching high-volume application. The candidates for the next-generation 32-nm node devices include ultra-high NA lens immersion lithography using a liquid having a higher refractive index than water in combination with a high refractive index lens and a high refractive index resist film, extreme ultraviolet ray (EUV) lithography of wavelength 13.5 nm, and double exposure (double patterning) version of the ArF lithography, on which active research efforts have been made.

As the miniaturization progresses and approaches the diffraction limit of light, light contrast decreases. In the case of a positive resist film, a decrease in light contrast leads to reductions of resolution and focus margin of hole and trench patterns.

As the patterns are miniaturized, the line width roughness (LWR) of line patterns and the critical dimension uniformity (CDU) of hole patterns are regarded significant. It is pointed out that these factors are affected by the segregation or agglomeration of a base polymer and acid generator and the diffusion of generated acid. Furthermore, there is a tendency that as the resist film becomes thinner, LWR becomes greater, and film thickness reduction to comply with the progress of miniaturization causes degradation of LWR, which is a serious problem.

Resist compositions for EUV lithography need to meet high sensitivity, high resolution, and low LWR at the same time. As the acid diffusion distance is reduced, LWR is reduced, but sensitivity becomes lower. For example, when the post-exposure baking (PEB) temperature is lowered, the outcome is a reduced LWR, but a lower sensitivity. When the amount of quencher added is increased, the outcome is a reduced LWR, but a lower sensitivity. It is necessary to overcome the tradeoff relation between sensitivity and LWR.

To suppress acid diffusion, Patent Documents 1 and 2 propose resist compounds containing repeating units derived from an onium salt of a sulfonic acid having a polymerizable unsaturated bond. The so-called polymer-bound acid generator is capable of generating a polymer-type sulfonic acid upon exposure and characterized by a very short distance of acid diffusion. Meanwhile, sensitivity may be enhanced by increasing the proportion of the acid generator. In the case of addition-type acid generators, as the amount of acid generator added is increased, a higher sensitivity is achievable, but the acid diffusion distance is also increased. Since the acid diffusion is non-uniform, increased acid diffusion leads to degraded LWR and CDU. With respect to a balance of sensitivity, LWR and CDU, the polymer-type acid generator has a high capability.

Iodine atoms very greatly absorb EUV having a wavelength of 13.5 nm, and an effect that secondary electrons are generated from iodine atoms during exposure is observed. Therefore, iodine atoms are attracting attention in EUV lithography. Patent Document 3 discloses a photo-acid generator having an iodine atom introduced into the anion, and Patent Document 4 discloses a polymerizable-group-containing photo-acid generator having an iodine atom introduced into the anion. Patent Document 5 discloses a photo-acid generator having an iodine atom introduced into both the cation and the anion. Although improvement in lithography performance to some extent by the above-described means is observed, iodine atoms do not have high organic solvent solubility, and there is concern about precipitation in the solvent.

Patent Document 6 discloses a photo-acid generator having multiple fluorine atoms introduced into the cation. Although the solvent solubility of the photo-acid generator is improved by the introduction of multiple fluorine atoms, the photo-acid generator is inadequate from the viewpoint of absorption of EUV, and there is still room for improvement.

Patent Documents 7 to 11 disclose photo-acid generators and quenchers (acid diffusion controllers) containing iodine atoms and fluorine atoms in the cations. Meanwhile, Patent Documents 12 to 15 disclose onium-salt-type monomers having iodine atoms and polymerizable groups introduced into the cations. Furthermore, Patent Documents 16 and 17 disclose onium-salt-type monomers having iodine atoms and polymerizable groups introduced into the anions. By virtue of these developments, improvement in performance as a resist material has been observed, but is still insufficient from the viewpoint of acid diffusion control, and there are demands for further development of a resist material useful for fine pattern formation.

### CITATION LIST

### Patent Literature

Patent Document 1: JP 4425776 B2
Patent Document 2: WO 2023/063203 A1
Patent Document 3: JP 6720926 B2
Patent Document 4: JP 6973274 B2
Patent Document 5: JP 7041204 B2
Patent Document 6: JP 7389562 B2
Patent Document 7: JP 2021-123579 A
Patent Document 8: JP 2021-123580 A
Patent Document 9: JP 2022-123839 A
Patent Document 10: JP 2023-88869 A
Patent Document 11: JP 2023-88870 A
Patent Document 12: JP 2022-28615 A
Patent Document 13: JP 2023-93372 A
Patent Document 14: JP 2023-165660 A
Patent Document 15: JP 2023-171323 A
Patent Document 16: JP 6973274 B2
Patent Document 17: WO 2024/014462 A1

### SUMMARY OF INVENTION

### Technical Problem

In the field of acid-catalyzed chemically-amplified resist compositions, there are demands for the development of a resist composition that has higher sensitivity, allows the improvement of lithography performance, such as exposure latitude (EL), LWR, CDU, and depth of focus (DOF), and is also excellent in etching resistance after patterning.

The present invention has been made in view of the above-described circumstances. An object of the present invention is to provide: an onium-salt-type monomer and a monomeric photo-acid generator that can be used for a chemically-amplified resist composition in the field of photolithography, the composition having excellent solvent solubility, high sensitivity, and high contrast, having excellent lithography performance, such as EL, LWR, CDU, and DOF, being resistant to pattern collapse even in fine pattern formation, and also having excellent etching resistance; a polymer including a repeating unit that is a derivative of the onium-salt-type monomer; a chemically-amplified resist composition containing the polymer; and a patterning process using the chemically-amplified resist composition.

### Solution to Problem

To achieve the object, the present invention provides an onium-salt-type monomer represented by the following formula (a),

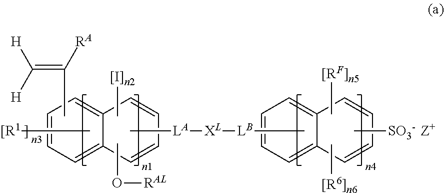

wherein “n1” represents 0 or 1, “n2” represents 1, 2, 3, or 4, “n3” represents 0, 1, or 2, provided that, when “n1” is 0, 1 n2+n3≤3 and when “n1” is 1, 1≤n2+n3≤5, “n4” represents 0 or 1, “n5” represents 0, 1, 2, 3, or 4, and “n6” represents 0, 1, or 2, provided that when “n4” is 0, 0≤n5+n6≤4 and when “n4” is 1, 0≤n5+n6≤6;
R^{A} represents a hydrogen atom, a fluorine atom, a methyl group, or a trifluoromethyl group;
R^{AL} represents an acid-labile group;
R¹ represents a halogen atom other than an iodine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “n3” is 2, the R's being identical to or different from each other, and the two R's optionally being bonded to each other to form a ring together with the carbon atoms bonded thereto;
R² represents a halogen atom other than a fluorine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “n6” is 2, the R²s being identical to or different from each other, and the two R²s optionally being bonded to each other to form a ring together with the carbon atoms bonded thereto;
R^{F} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms, when “n5” is 2, 3, or 4, the R^{F}s being identical to or different from each other;
L^{A} and L^{B} each independently represent a single bond, an ether bond, an ester bond, a sulfonic acid ester bond, an amide bond, a sulfonic acid amide bond, a carbonate bond, or a carbamate bond;
X^{L} represents a single bond or a hydrocarbylene group having 1 to 40 carbon atoms and optionally containing a heteroatom; and
Z⁺ represents an onium cation.

Such an onium-salt-type monomer can be used for a chemically-amplified resist composition that is excellent in solvent solubility, has high sensitivity and high contrast, is excellent in lithography performance, such as EL, LWR, CDU, and DOF, is also resistant to pattern collapse in fine patterning, and is also excellent in etching resistance, particularly in photolithography using a high-energy beam such as KrF excimer laser beam, ArF excimer laser beam, electron beam (EB), and EUV.

The inventive onium-salt-type monomer is preferably represented by the following formula (a1),

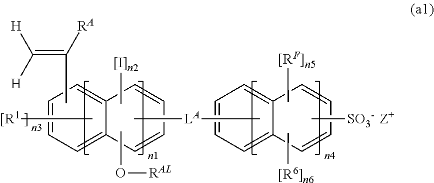

wherein “n1” to “n6”, R^{A}, R^{AL}, R¹, R², R^{F}, L^{A}, and Z⁺ are as defined above.

The inventive onium-salt-type monomer is more preferably represented by the following formula (a2),

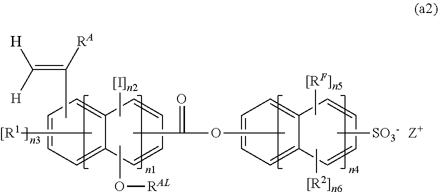

wherein “n1” to “n6”, R^{A}, R^{AL}, R¹, R², R^{F}, and Z⁺ are as defined above.

It is suitable for the inventive onium-salt-type monomer to have such structures.

The acid-labile group represented by R^{AL} preferably has a structure represented by the following formula (AL-1) or (AL-2),

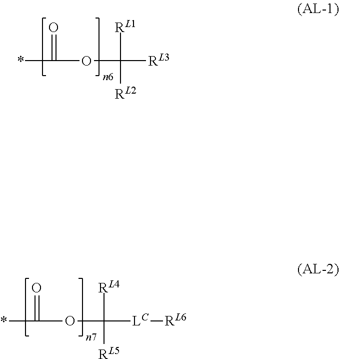

wherein “n6” represents 0 or 1 and “n7” represents 0 or 1;
R^{L1}, R^{L2}, and R^{L3} each independently represent a hydrocarbyl group having 1 to 12 carbon atoms, part of —CH₂— of the hydrocarbyl group optionally being substituted with —O— or —S—, when the hydrocarbyl group contains an aromatic ring, part or all of hydrogen atoms of the aromatic ring optionally being substituted with a halogen atom, a cyano group, a nitro group, an alkyl group having 1 to 4 carbon atoms and optionally containing a halogen atom, or an alkoxy group having 1 to 4 carbon atoms and optionally containing a halogen atom, and the R^{L1} and the R^{L2} optionally being bonded to each other to form a ring together with the carbon atom bonded thereto, part of —CH₂— of the ring optionally being substituted with —O— or —S—;
R^{L4} and R^{L5} each independently represent a hydrogen atom or a hydrocarbyl group having 1 to 10 carbon atoms and optionally containing a halogen atom, and R^{L6} represents a hydrocarbyl group having 1 to 20 carbon atoms and optionally containing a halogen atom, part of —CH₂— of the hydrocarbyl group optionally being substituted with —O— or —S—, and the R^{L5} and the R^{L6} optionally being bonded to each other to form a heterocyclic group having 3 to 20 carbon atoms together with the carbon atom and the L^{C} bonded thereto, part of the —CH₂— of the heterocyclic group optionally being substituted with —O— or —S—;
L^{C} represents —O— or —S—; and
“*” represents an attachment point to the adjacent —O—.

When the acid-labile group has such a structure, the advantageous effects of the present invention can be exhibited more sufficiently.

The onium cation represented by Z⁺ is preferably a sulfonium cation represented by the following formula (Z-1) or an iodonium cation represented by the following formula (Z-2),

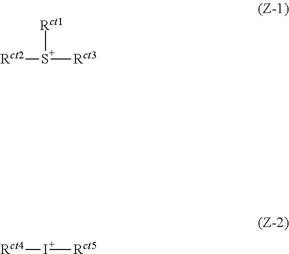

wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

Alternatively, the onium cation represented by Z⁺ may be a sulfonium cation represented by the following formula (Z-3),

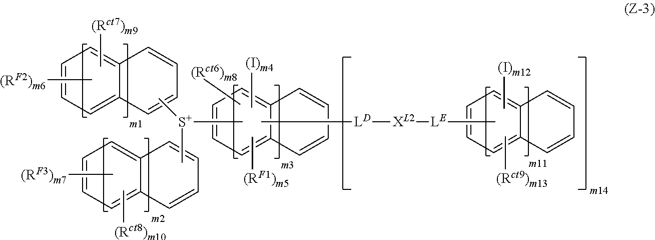

wherein “m1” represents 0 or 1, “m2” represents 0 or 1, “m3” represents 0 or 1, “m4” represents 0, 1, 2, 3, or 4, “m5” represents 0, 1, 2, 3, or 4, “m6” represents 0, 1, 2, 3, 4, 5, or 6, “m7” represents 0, 1, 2, 3, 4, 5, or 6, “m8” represents 0, 1, or 2, “m9” represents 0, 1, or 2, “m10” represents 0, 1, or 2, “m11” represents 0 or 1, “m12” represents 0, 1, 2, 3, or 4, “m13” represents 0, 1, or 2, and “m14” represents 0, 1, or 2, provided that, when “m1” is 0, 0 m6+m9≤4 and when “m1” is 1, 0 m6+m9≤6, when “m2” is 0, 0 m7+m10≤4 and when “m2” is 1, 0 m7+m10≤6, when “m3” is 0, 1 m4+m5+m8+m14≤4 and when “m3” is 1, 1 m4+m5+m8+m14≤6, when “m11” is 0, 0 m12+m13≤4 and when “m11” is 1, 0 m12+m13≤6, and m4+m12≤1;
R^{F1} to R^{F3} each independently represent a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms, when “m5” is 2 or more, the R^{F1}s being identical to or different from each other, when “m6” is 2 or more, the R^{F2}s being identical to or different from each other, and when “m7” is 2 or more, the R^{F3}s being identical to or different from each other;
R^{ct6} to R^{ct9} each represent a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m8” is 2, the two R^{ct6}s being identical to or different from each other and the two R^{ct6}s optionally being bonded to each other to form a ring together with the carbon atoms bonded thereto, when “m9” is 2, the two R^{ct7}s being identical to or different from each other and the two R^{ct7}s optionally being bonded to each other to form a ring together with the carbon atoms bonded thereto, when “m10” is 2, the two R^{ct8}s being identical to or different from each other and the two R^{ct8}s optionally being bonded to each other to form a ring together with the carbon atoms bonded thereto, and when “m13” is 2, the two R^{ct9}s being identical to or different from each other and the two R^{ct9}s optionally being bonded to each other to form a ring together with the carbon atoms bonded thereto;
the aromatic rings bonded directly to the S⁺ in the sulfonium cation are optionally bonded to each other to form a ring together with the S⁺;
L^{D} and L^{E} each independently represent a single bond, an ether bond, an ester bond, an amide bond, a sulfonic acid ester bond, a sulfonic acid amide bond, a carbonate bond, or a carbamate bond; and
X^{L2} represents a single bond or a hydrocarbylene group having 1 to 40 carbon atoms and optionally containing a heteroatom.

In the present invention, such an onium cation can be suitably used.

The present invention also provides a monomeric photo-acid generator comprising the above-described onium-salt-type monomer.

The inventive onium-salt-type monomer itself can be used as a photo-acid generator.

The present invention also provides a polymer comprising a repeating unit which is a derivative of the above-described onium-salt-type monomer.

The inventive polymer is a polymer-bound photo-acid generator that functions as a base polymer in a chemically-amplified resist composition, and also functions as a photo-acid generator.

The inventive polymer can further comprise a repeating unit represented by the following formula (b1) or (b2),

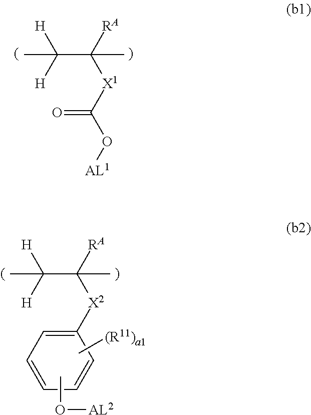

wherein each R^{A} independently represents a hydrogen atom, a fluorine atom, a methyl group, or a trifluoromethyl group;
X¹ represents a single bond, a phenylene group, a naphthylene group, *—C(═O)—O—X¹¹—, or *—C(═O)—NH—X¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, and X¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring;
X² represents a single bond, *—C(═O)—O—, or *—C(═O)—NH—;
“*” represents an attachment point to the carbon atom of the main chain;
R¹¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom;
AL¹ and AL² each independently represent an acid-labile group; and
“a1” represents 0, 1, 2, 3, or 4.

The inventive polymer can further comprise a repeating unit represented by the following formula (b3),

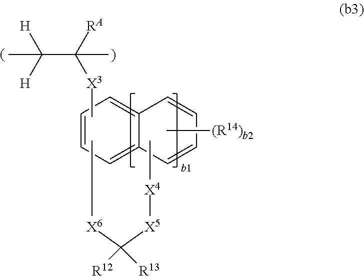

wherein “b1” represents 0 or 1, and “b2” represents 0, 1, 2, or 3 when “b1” is 0 and represents 0, 1, 2, 3, 4, or 5 when “b1” is 1;
R^{A} represents a hydrogen atom, a fluorine atom, a methyl group, or a trifluoromethyl group;
X³ represents a single bond, *—C(═O)—O—, or *—C(═O)—NH—, and “*” represents an attachment point to the carbon atom of the main chain;
R¹² and R¹³ each independently represent a hydrogen atom or a hydrocarbyl group having 1 to 20 carbon atoms and optionally containing a heteroatom, the R¹² and the R¹³ optionally being bonded to each other to form a ring together with the carbon atom bonded thereto;
R¹⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A})(R^{14B}), and R^{14A} and R^{14B} each independently represent a hydrogen atom or a hydrocarbyl group having 1 to 6 carbon atoms, when “b2” is 2 or more, the R¹⁴s optionally being bonded to each other to form a ring together with the carbon atoms of the aromatic ring bonded thereto;
X⁴ represents a single bond, an aliphatic hydrocarbylene group having 1 to 4 carbon atoms, a carbonyl group, a sulfonyl group, or a group which is a combination of the groups; and
X⁵ and X⁶ each independently represent an oxygen atom or a sulfur atom, provided that the X⁴ and the X⁶ are bonded to adjacent carbon atoms of the aromatic ring.

The inventive polymer can further comprise a repeating unit represented by the following formula (c),

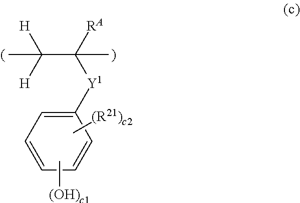

wherein R^{A} represents a hydrogen atom, a fluorine atom, a methyl group, or a trifluoromethyl group;
Y¹ represents a single bond, *—C(═O)—O—, or *—C(═O)—NH—, and “*” represents an attachment point to the carbon atom of the main chain;
R²¹ represents a halogen atom, a nitro group, a cyano group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
“c1” represents 1, 2, 3, or 4 and “c2” represents 0, 1, 2, or 3, provided that 1≤c1+c2≤5.

The inventive polymer can further comprise a repeating unit represented by the following formula (d),

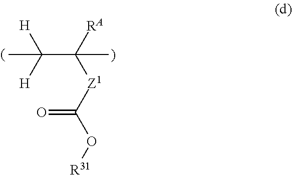

wherein R^{A} represents a hydrogen atom, a fluorine atom, a methyl group, or a trifluoromethyl group;
Z¹ represents a single bond, a phenylene group, a naphthylene group, *—C(═O)—O—Z¹¹—, or *—C(═O)—NH—Z¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, “*” represents an attachment point to the carbon atom of the main chain, and Z¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; and
R³¹ represents a hydrogen atom or a group having 1 to 20 carbon atoms and containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—).

In the inventive polymer, various repeating units, such as those described above, can be copolymerized as necessary.

The present invention also provides a chemically-amplified resist composition comprising (A) a base polymer comprising the above-described polymer.

The inventive polymer is a polymer-bound photo-acid generator that functions as a base polymer in a chemically-amplified resist composition, and also functions as a photo-acid generator.

The inventive chemically-amplified resist composition preferably further comprises one or more selected from (B) an organic solvent, (C) a quencher, (D) an acid generator, (E) a surfactant, and (F) a dissolution inhibitor.

The inventive chemically-amplified resist composition can contain various additives as necessary.

The present invention also provides a patterning process comprising the steps of:
forming a resist film by using the above-described chemically-amplified resist composition on a substrate;
exposing the resist film by using a high-energy beam; and
developing the exposed resist film by using a developer.

According to the inventive patterning process, a pattern can be formed with high sensitivity and high contrast, particularly in photolithography using a high-energy beam such as KrF excimer laser beam, ArF excimer laser beam, electron beam (EB), and EUV.

The high-energy beam is preferably an ArF excimer laser beam having a wavelength of 193 nm, a KrF excimer laser beam having a wavelength of 248 nm, an electron beam, or an extreme ultraviolet ray having a wavelength of 3 to 15 nm.

In the inventive patterning process, such high-energy beams can be suitably used.

### Advantageous Effects of Invention

A polymer that includes a repeating unit derived from an onium-salt-type monomer having a structure in which an aromatic ring substituted with a polymerizable group and an iodine atom is included and the aromatic ring is further bonded with a substituent including an aromatic sulfonate anion structure and an acid-labile group, and generating an acid on exposure has favorable solvent solubility by virtue of its branched structure. In EUV lithography of wavelength 13.5 nm, secondary electrons are generated from iodine atoms during exposure, since iodine atoms very greatly absorb EUV. In the aromatic sulfonate anion bonded to the aromatic ring substituted with the polymerizable group and the iodine atom, the distance from the polymer main chain to the acid generation position is short, and secondary electrons generated from the iodine atom promote the decomposition of the cation, located near the anion. Thus, an acid is generated efficiently, enhancing sensitivity. Furthermore, the atomic weight of an iodine atom is high and the polymer has a structure where the generated acid is bonded to the polymer main chain, and therefore, the polymer has a characteristic that acid diffusion is low. The acid-labile group contained in the anion has a structure where an aromatic hydroxy group is protected, and a deprotection reaction progresses along with the generation of the acid. Aromatic hydroxy groups thus produced have enhanced acidity by virtue of the electron-withdrawing effect of the iodine atoms, and have a high affinity with alkaline developers. Accordingly, in a case where exposed portions are developed with an alkaline developer, polymer residues are easily dissolved, and the dissolution contrast between exposed and unexposed portions is enhanced. And by the combined effect of these effects, LWR and CDU can be improved. Furthermore, the aromatic ring in the repeating unit acts as a favorable etching resistance group, and is suitable in fine patterning.

### DESCRIPTION OF EMBODIMENTS

As described above, there have been demands for the development of: an onium-salt-type monomer that can be used for a chemically-amplified resist composition in the field of photolithography, the composition having excellent solvent solubility, high sensitivity, and high contrast, having excellent lithography performance, such as EL, LWR, CDU, and DOF, being resistant to pattern collapse even in fine pattern formation, and also having excellent etching resistance; a polymer including a repeating unit that is a derivative of the onium-salt-type monomer; a chemically-amplified resist composition containing the polymer; and a patterning process using the chemically-amplified resist composition.

To achieve the object, the present inventor has studied earnestly and found out that a polymer that contains a repeating unit that is a derivative of an onium-salt-type monomer having an aromatic ring substituted with a polymerizable group and an iodine atom and having a structure where a substituent including an aromatic sulfonate anion structure and an acid-labile group are further bonded to the aromatic ring has favorable solvent solubility, and when the polymer is contained as a polymer-bound photo-acid generator, it is possible to obtain a chemically-amplified resist composition having high sensitivity, high contrast, high resolution, improved lithography performance, such as LWR and CDU, and also excellent etching resistance after patterning. Thus, the present invention has been completed.

That is, the present invention is an onium-salt-type monomer represented by the formula (a).

Hereinafter, the present invention will be described in detail, but the present invention is not limited thereto.

### [Onium-Salt-Type Monomer]

The inventive onium-salt-type monomer is represented by the following formula (a).

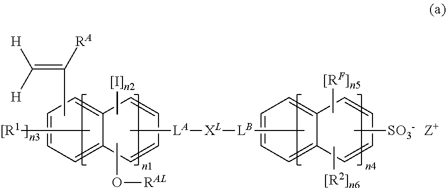

In the formula (a), “n1” represents 0 or 1. When “n1” is 0, a benzene ring is indicated and when “n1” is 1, a naphthalene ring is indicated, and from the viewpoint of solvent solubility, a benzene ring, where “n1” is 0, is preferable. “n2” represents 1, 2, 3, or 4. From the viewpoint of procuring raw materials, “n2” is preferably 1, 2, or 3, more preferably 1 or 2, and further preferably 1. “n3” represents 0, 1, or 2. However, when “n1” is 0, 1 n2+n3≤3 and when “n1” is 1, 1 n2+n3≤5.

In the formula (a), “n4” represents 0 or 1. When “n4” is 0, a benzene ring is indicated and when “n4” is 1, a naphthalene ring is indicated, and from the viewpoint of solvent solubility, a benzene ring, where “n4” is 0, is preferable. “n5” represents 0, 1, 2, 3, or 4, and when R^{F}, described later, is a fluorine atom, “n5” is preferably 4. “n6” represents 0, 1, or 2. However, when “n4” is 0, 0 n5+n6≤4 and when “n4” is 1, 0≤n5+n6≤6.

In the formula (a), R^{A} represents a hydrogen atom, a fluorine atom, a methyl group, or a trifluoromethyl group. Among these, a hydrogen atom and a methyl group are preferable, and a hydrogen atom is more preferable.

In the formula (a), R^{AL} represents an acid-labile group. As the acid-labile group, a group represented by the following formula (AL-1) or (AL-2) is preferable.

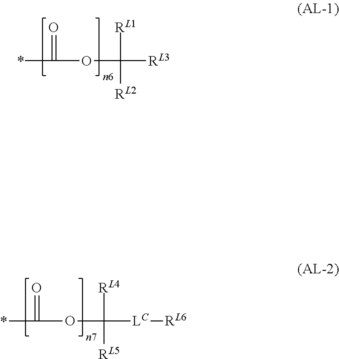

In the formulae, “*” represents an attachment point to the —O—.

In the formula (AL-1), “n6” represents 0 or 1. R^{L1}, R^{L2}, and R^{L3} each independently represent a hydrocarbyl group having 1 to 12 carbon atoms, part of —CH₂— of the hydrocarbyl group optionally being substituted with —O— or —S—, when the hydrocarbyl group contains an aromatic ring, part or all of hydrogen atoms of the aromatic ring optionally being substituted with a halogen atom, a cyano group, a nitro group, an alkyl group having 1 to 4 carbon atoms and optionally containing a halogen atom, or an alkoxy group having 1 to 4 carbon atoms and optionally containing a halogen atom, and the R^{L1} and the R^{L2} optionally being bonded to each other to form a ring together with the carbon atom bonded thereto, part of —CH₂— of the ring optionally being substituted with —O—or —S—.

In the formula (AL-2), “n7” represents 0 or 1. R^{L4} and R^{L5} each independently represent a hydrogen atom or a hydrocarbyl group having 1 to 10 carbon atoms and optionally containing a halogen atom, and R^{L6} represents a hydrocarbyl group having 1 to 20 carbon atoms and optionally containing a halogen atom, part of —CH₂— of the hydrocarbyl group optionally being substituted with —O— or —S—, and the R^{L5} and the R^{L6} optionally being bonded to each other to form a heterocyclic group having 3 to 20 carbon atoms together with the carbon atom and the L^{C} bonded thereto, part of the —CH₂— of the heterocyclic group optionally being substituted with —O— or —S—. L^{C} represents —O— or —S—.

Specific examples of the acid-labile group represented by the formula (AL-1) include the following, but are not limited thereto. “*” represents an attachment point to the adjacent —O—.

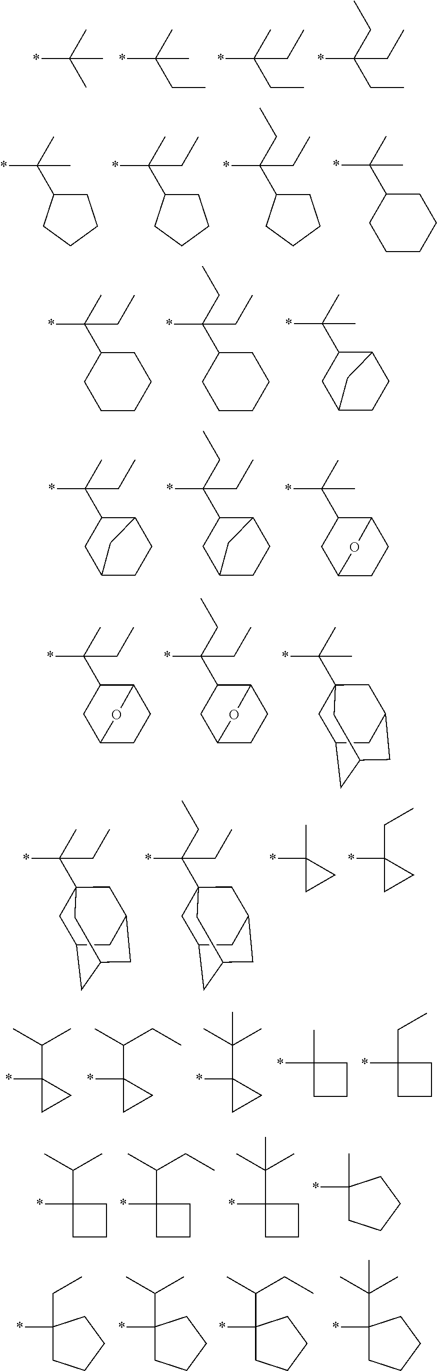

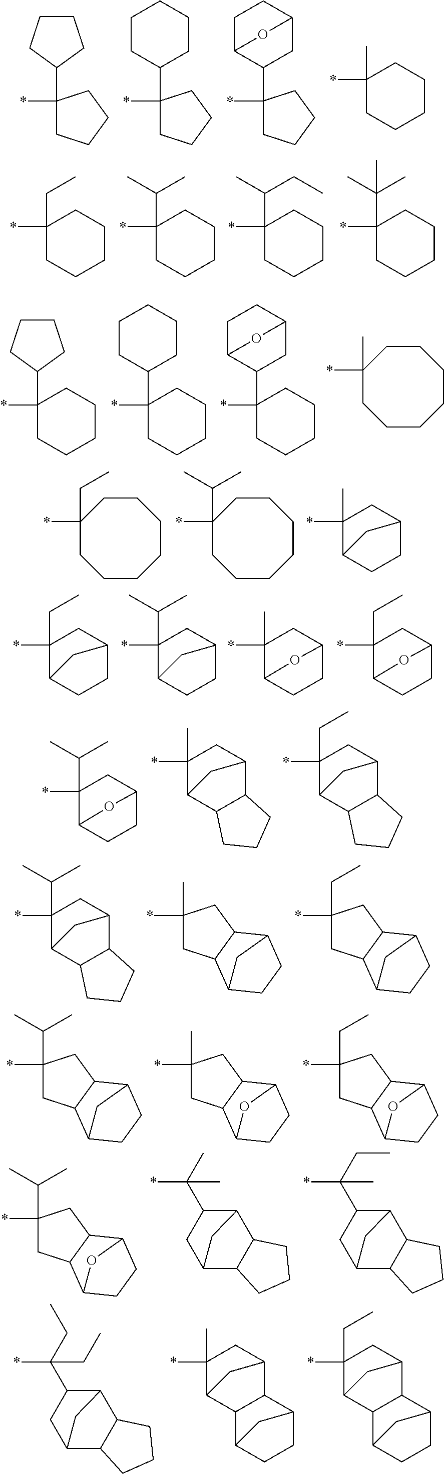

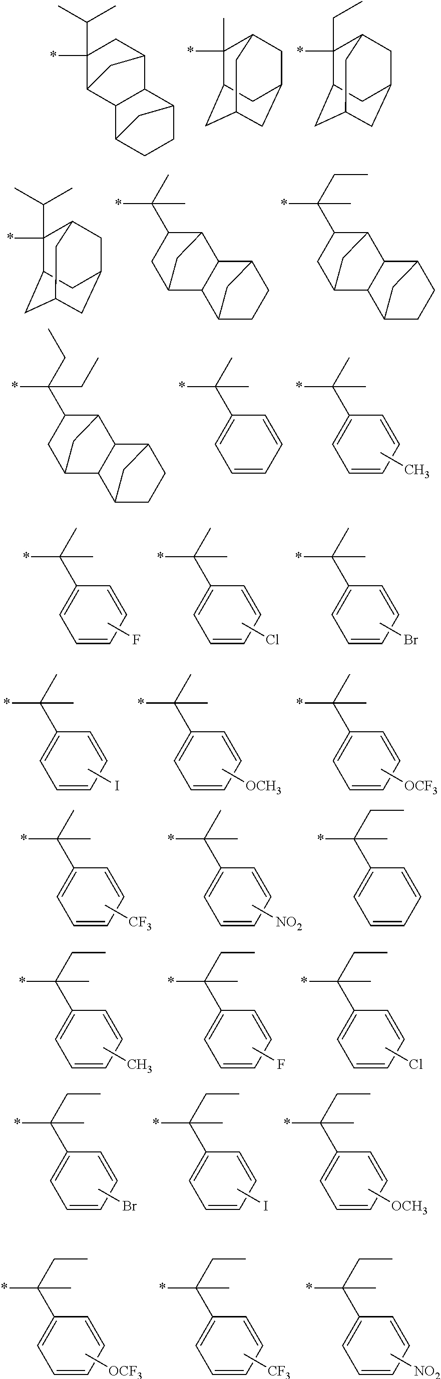

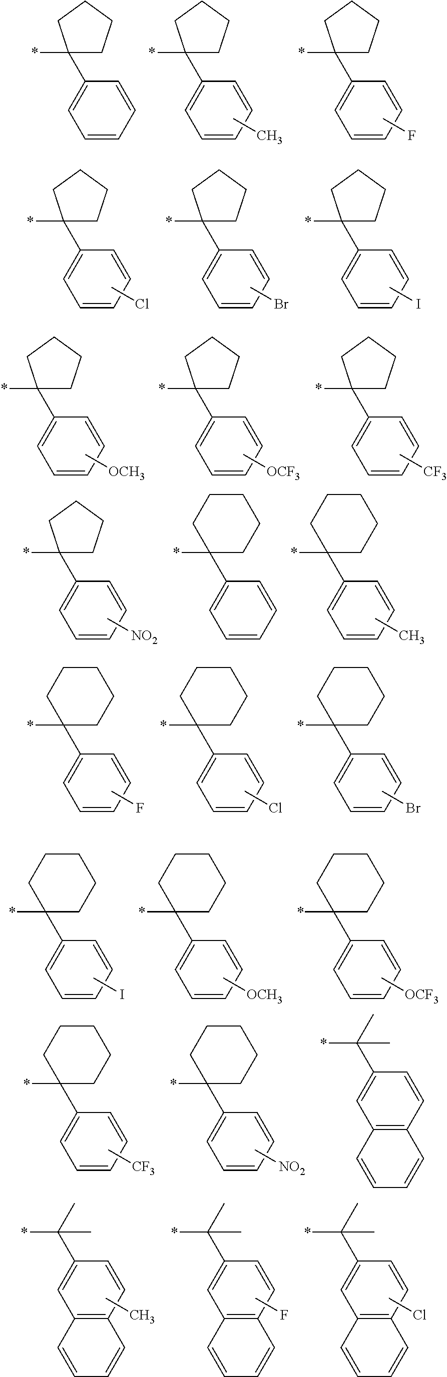

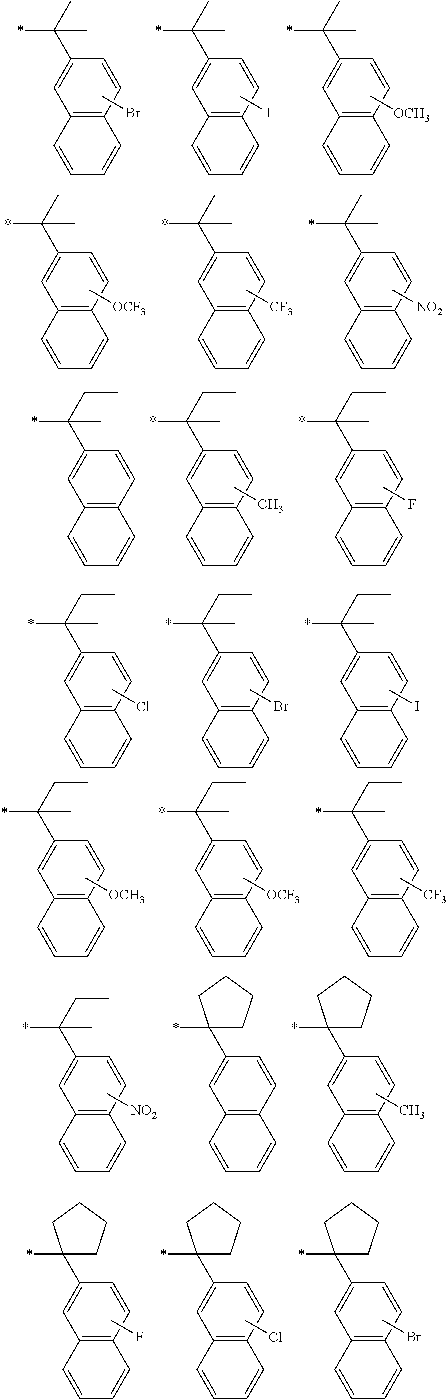

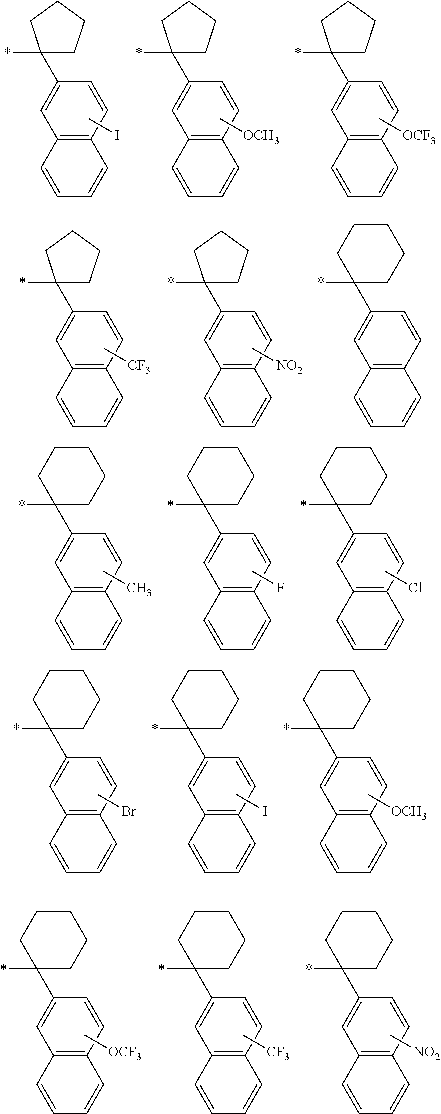

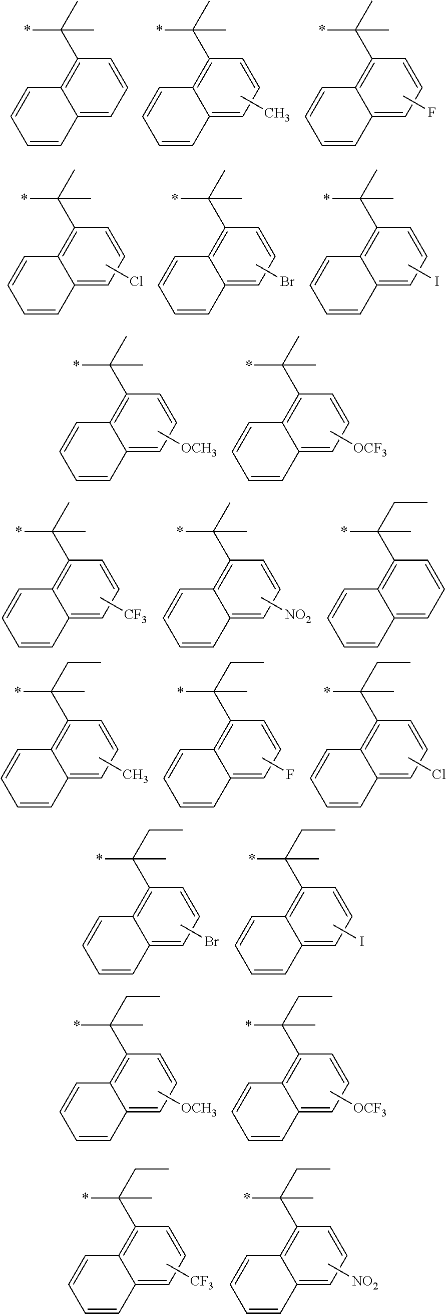

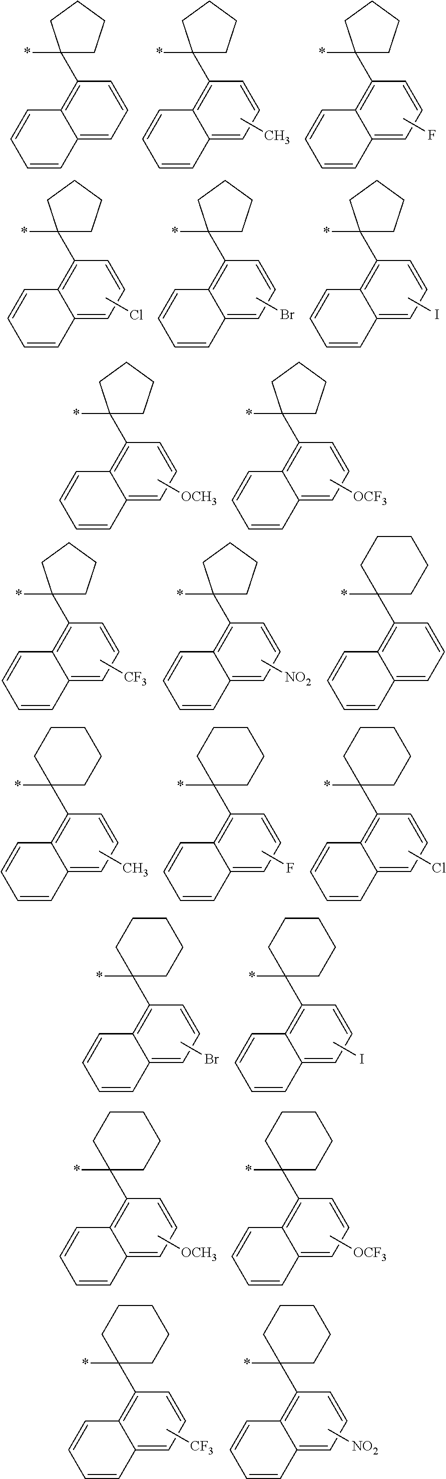

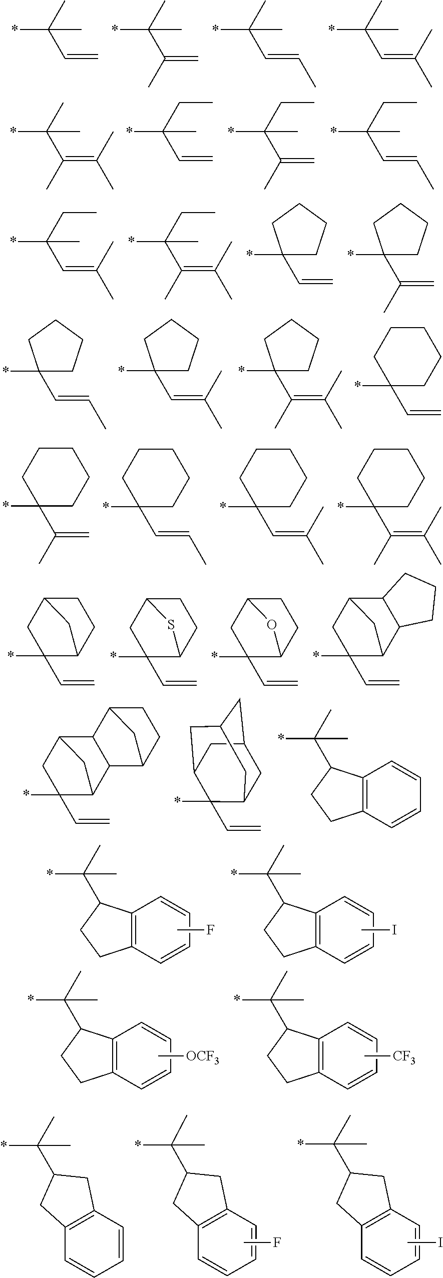

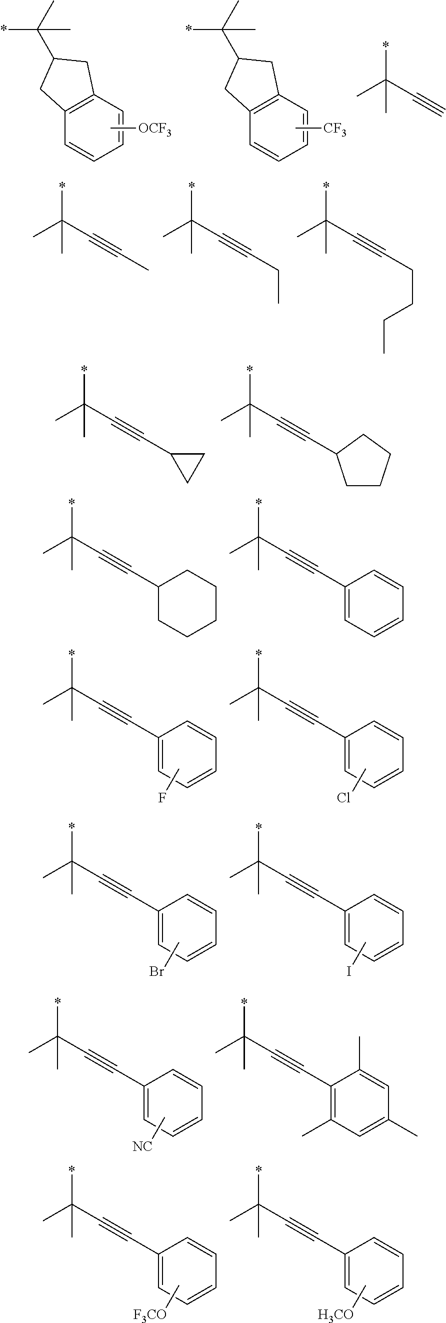

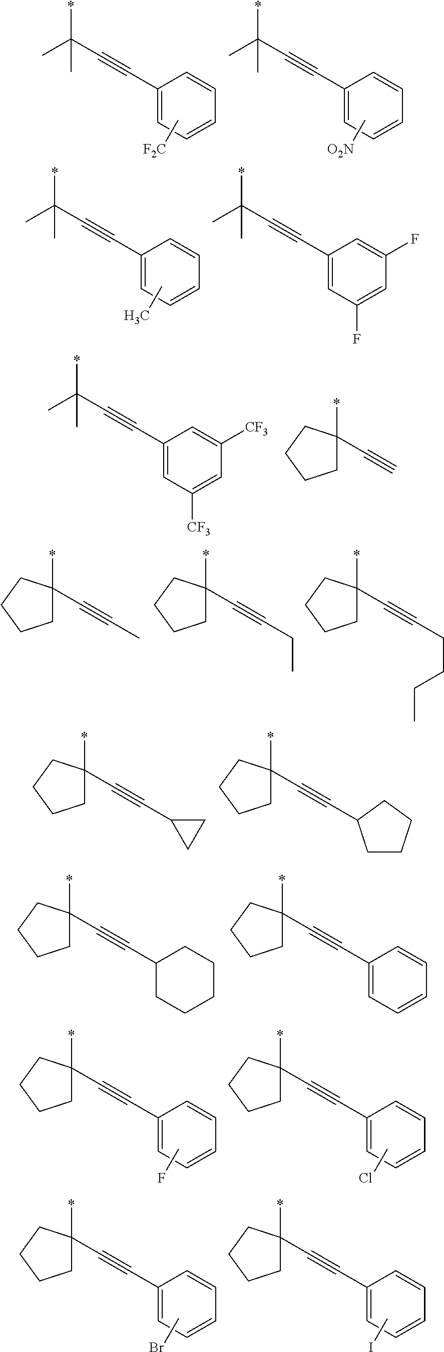

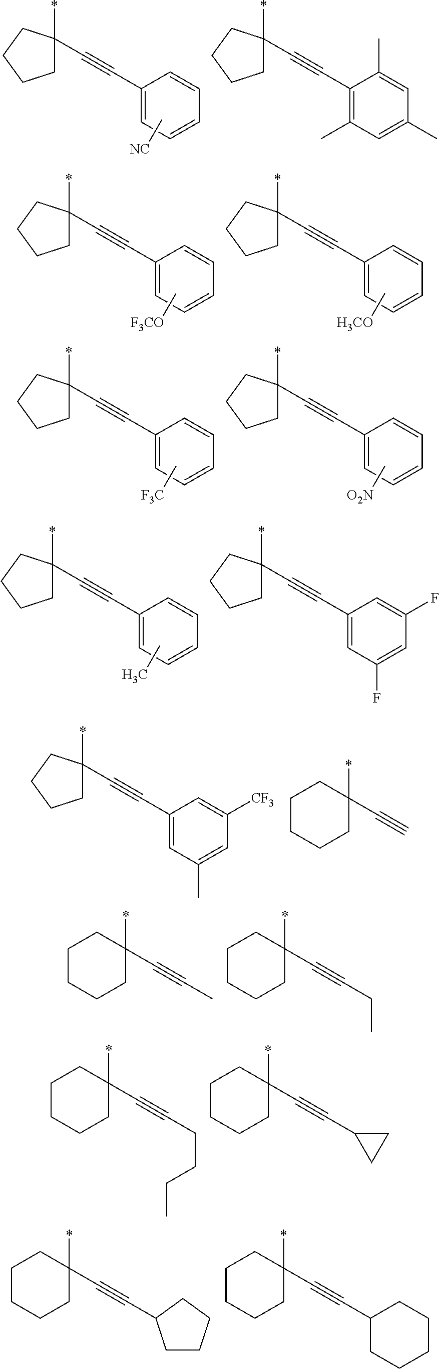

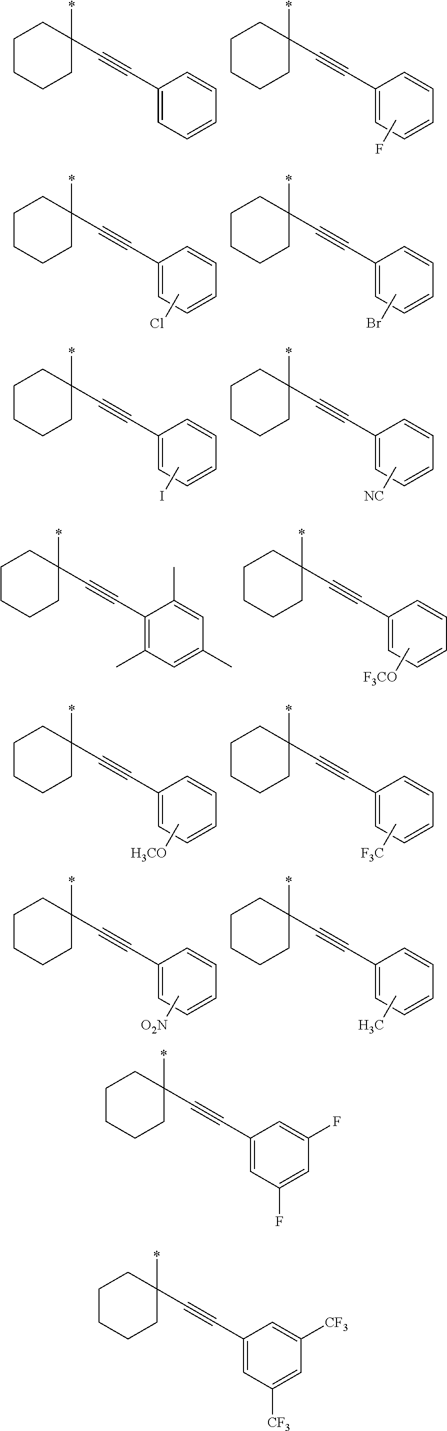

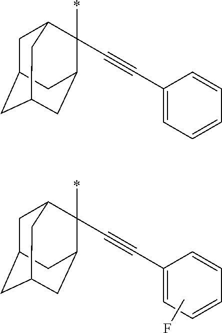

Specific examples of the acid-labile group represented by the formula (AL-2) include the following, but are not limited thereto. “*” represents an attachment point to the adjacent —O—.

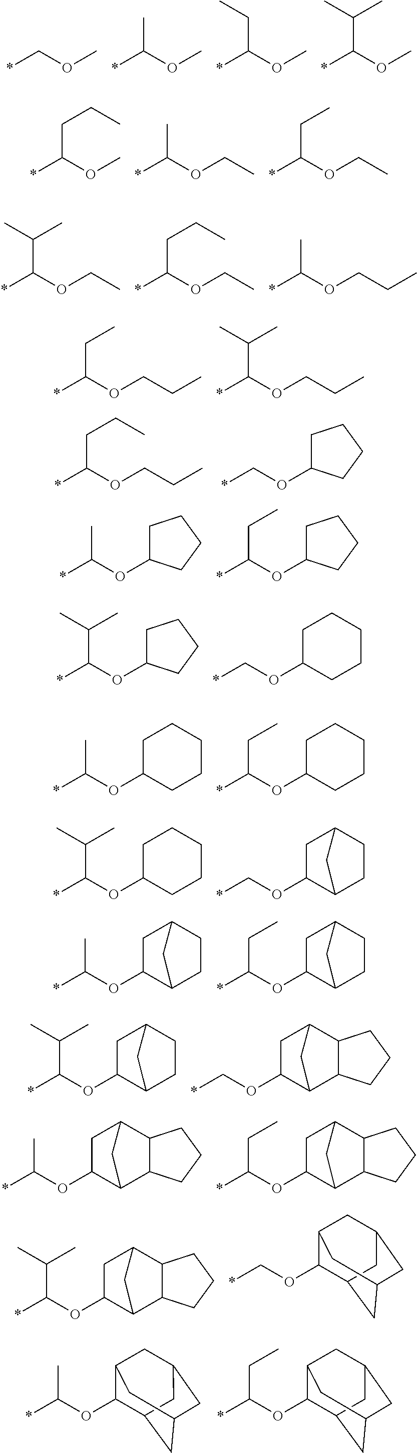

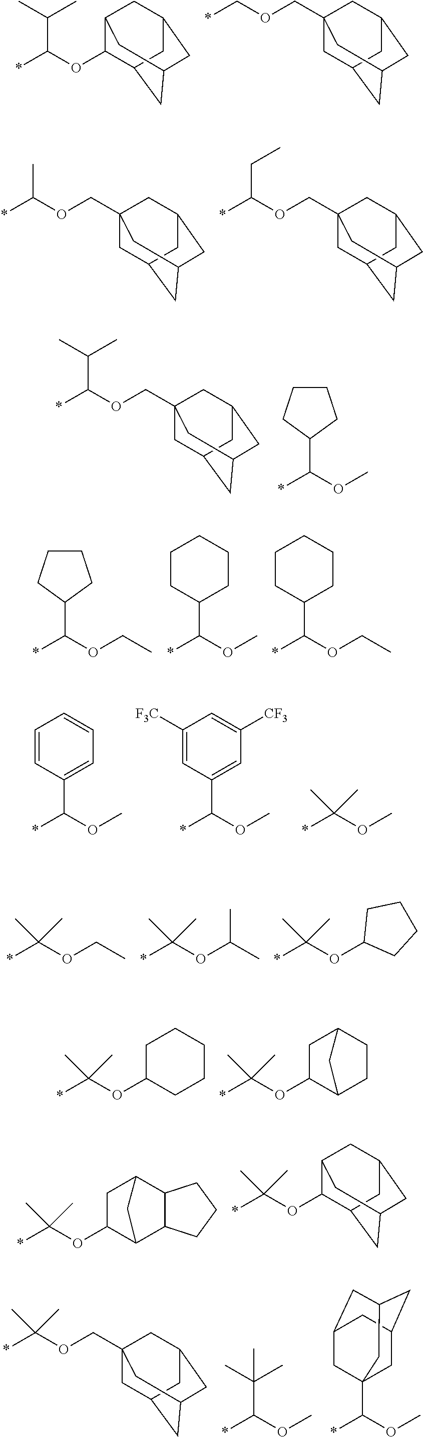

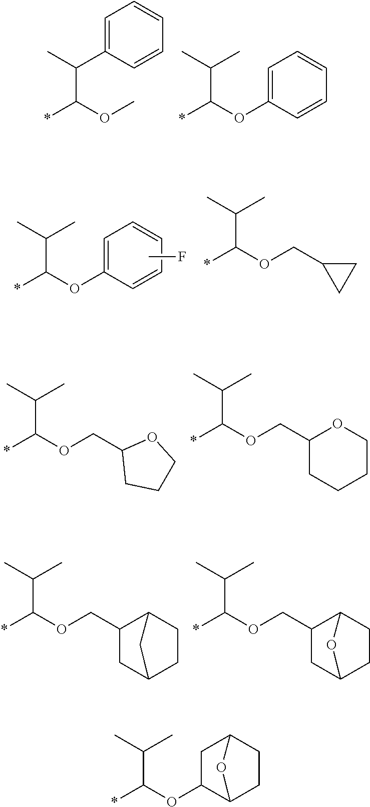

In the formula (a), R¹ represents a halogen atom other than an iodine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. As the halogen atom other than an iodine atom, a fluorine atom, a chlorine atom, or a bromine atom is preferable, and a fluorine atom is more preferable. The hydrocarbyl group and the hydrocarbyl moieties of the hydrocarbyloxy group and the hydrocarbylthio group may be saturated or unsaturated, and may be linear, branched, or cyclic. Specific examples thereof include: alkyl groups having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group and a naphthyl group; aralkyl groups having 7 to 20 carbon atoms, such as a benzyl group, a 1-phenylethyl group, and a 2-phenylethyl group; groups which are combinations of these groups; etc. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc. When “n3” is 2, the R's may be identical to or different from each other.

Furthermore, when “n3” is 2, the two R's are optionally bonded to each other to form a ring together with the carbon atoms bonded thereto. Specific examples of the ring formed in this case include a cyclopropane ring, a cyclobutane ring, a cyclopentane ring, a cyclohexane ring, a norbornane ring, and an adamantane ring. Furthermore, part or all of the hydrogen atoms in the ring may be substituted with a group containing a heteroatom, such as an oxygen atom, a sulfur atom, a nitrogen atom, or a halogen atom, and part of the —CH₂— in the ring may be substituted with a group containing a heteroatom such as an oxygen atom, a sulfur atom, or a nitrogen atom. The resulting ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

In the formula (a), R² represents a halogen atom other than a fluorine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. Specific examples of the halogen atom other than a fluorine atom include a chlorine atom, a bromine atom, and an iodine atom. The hydrocarbyl group and the hydrocarbyl moieties of the hydrocarbyloxy group and the hydrocarbylthio group may be saturated or unsaturated, and may be linear, branched, or cyclic. Specific examples thereof include those given as examples of the hydrocarbyl group represented by R¹, but are not limited thereto. When “n6” is 2, the R²s may be identical to or different from each other.

Furthermore, when “n6” is 2, the two R²s may be bonded to each other to form a ring together with the carbon atoms bonded thereto. As the ring, 5- to 8-membered rings are preferable.

In the formula (a), each R^{F} independently represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. Among these, a fluorine atom, a trifluoromethyl group, a trifluoromethoxy group, and a trifluoromethylthio group are preferable, and a fluorine atom is more preferable. When “n5” is 2, 3, or 4, the R^{F}s may be identical to or different from each other.

In the formula (a), L^{A} and L^{B} each independently represent a single bond, an ether bond, an ester bond, a sulfonic acid ester bond, an amide bond, a sulfonic acid amide bond, a carbonate bond, or a carbamate bond. Among these, the L^{A} is preferably a single bond, an ether bond, an ester bond, or a sulfonic acid ester bond, more preferably an ester bond or a sulfonic acid ester bond. The L^{B} is preferably a single bond, an ether bond, an ester bond, or a sulfonic acid ester bond, more preferably an ester bond or a sulfonic acid ester bond.

The L^{A} and the —O—R^{AL} are preferably bonded to adjacent carbon atoms of the aromatic ring. In this case, the substituent including the aromatic sulfonate anion structure and the acid-labile group are present in spatially closer positions, and therefore, it can be expected that a deprotection reaction progresses smoothly, thus, further enhancing dissolution contrast.

In the formula (a), each X^{L} independently represents a single bond or a hydrocarbylene group having 1 to 40 carbon atoms and optionally containing a heteroatom. The hydrocarbylene group may be linear, branched, or cyclic, and specific examples thereof include alkanediyl groups, cyclic saturated hydrocarbylene groups, and arylene groups. Specific examples of the heteroatom include an oxygen atom, a nitrogen atom, and a sulfur atom.

Specific examples of the hydrocarbylene group represented by X^{L} having 1 to 40 carbon atoms and optionally containing a heteroatom include the following, but are not limited thereto. Note that, in the following formulae, each “*” represents an attachment point to L^{A} and L^{B} respectively.

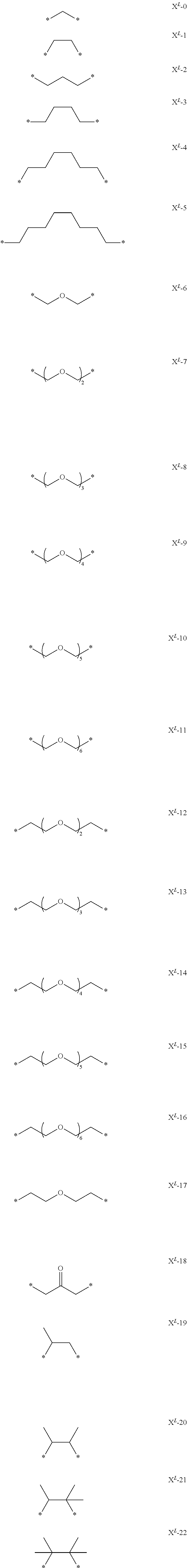

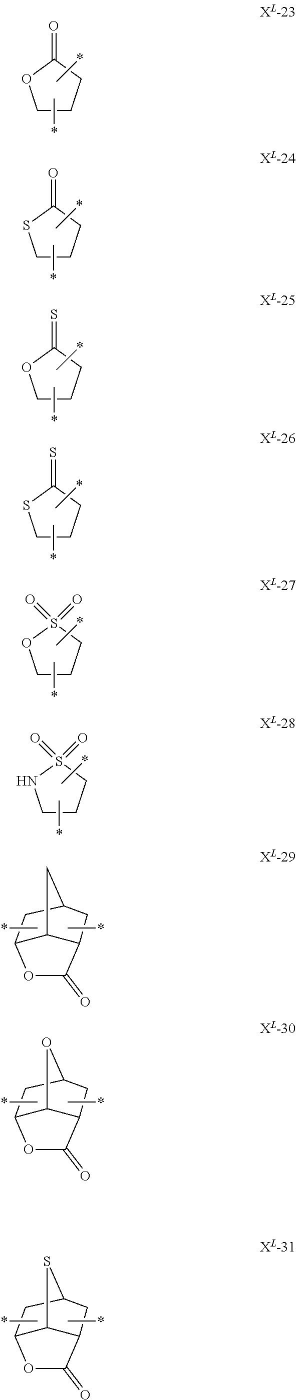

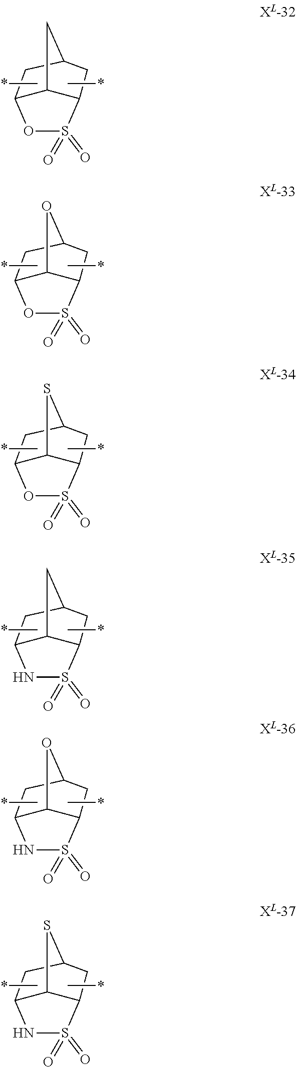

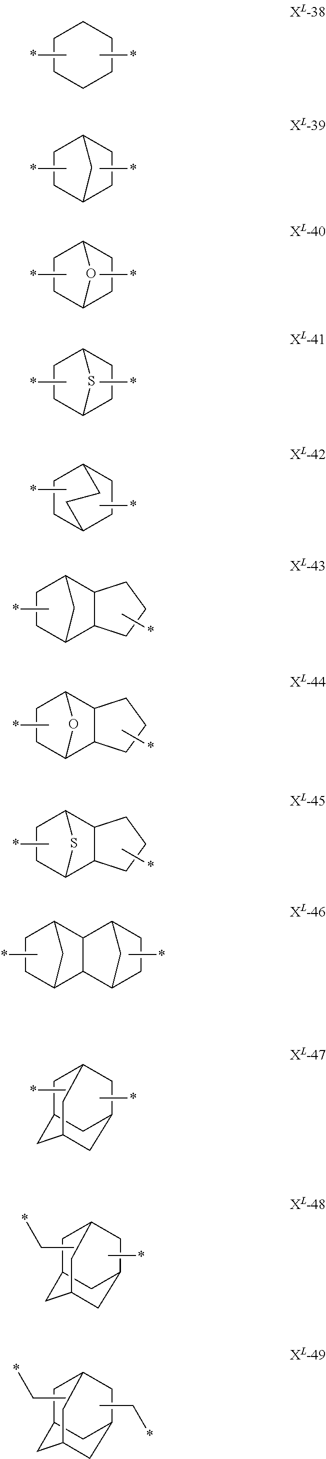

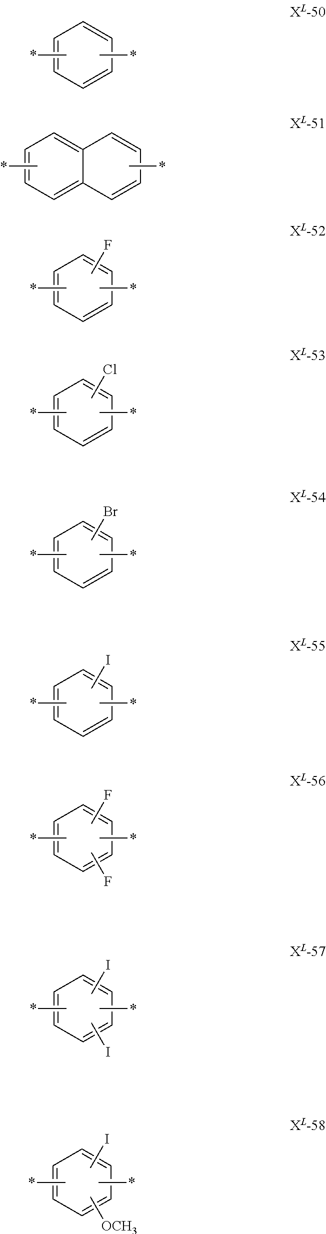

Among these, X^{L}-0 to X^{L}-22, X^{L}-29 to X^{L}-34, and X^{L}-47 to X^{L}-58 are preferable.

As the onium-salt-type monomer represented by the formula (a), one represented by the following formula (a1) is preferable.

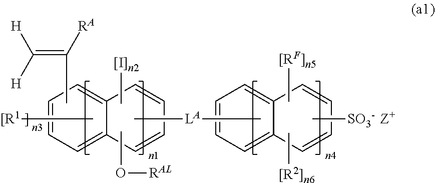

In the formula, “n1” to “n6”, R^{A}, R^{AL}, R¹, R², R^{F}, L^{A}, and Z⁺ are as defined above.

As the onium-salt-type monomer represented by the formula (a1), one represented by the following formula (a2) is preferable.

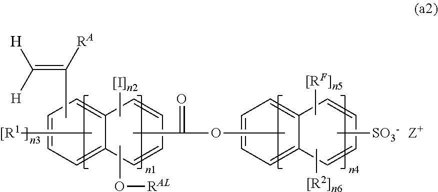

In the formula, “n1” to “n6”, R^{A}, R^{AL}, R¹, R², R^{F}, and Z⁺ are as defined above.

Examples of the anion of the onium-salt-type monomer represented by the formula (a) include the following, but are not limited thereto. Note that, in the following formulae, R^{A} is as defined above. Furthermore, the attachment positions of the substituents on the aromatic rings may change places with each other.

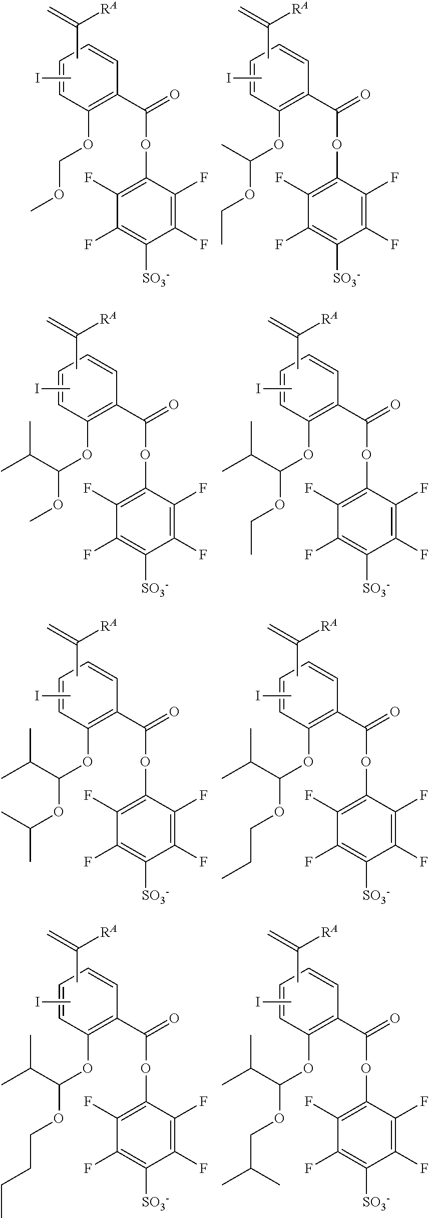

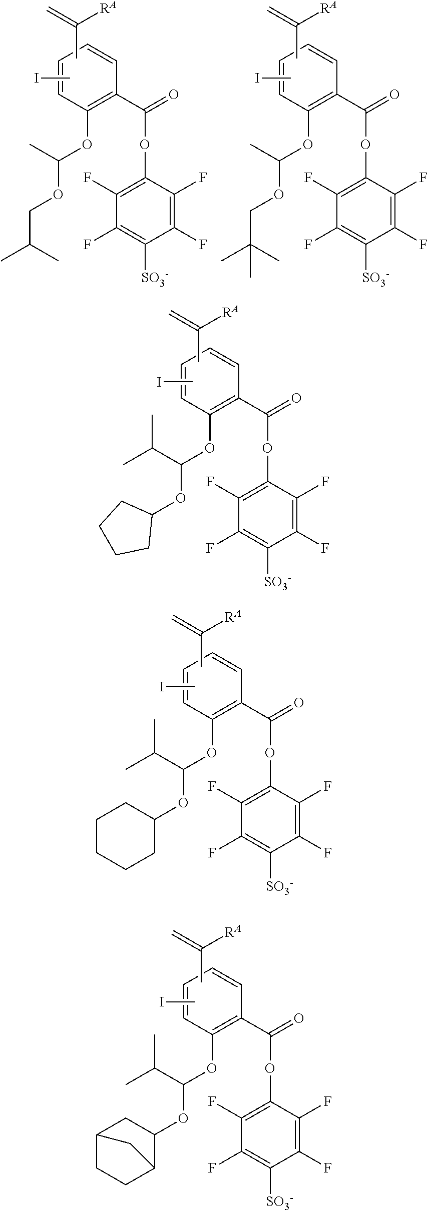

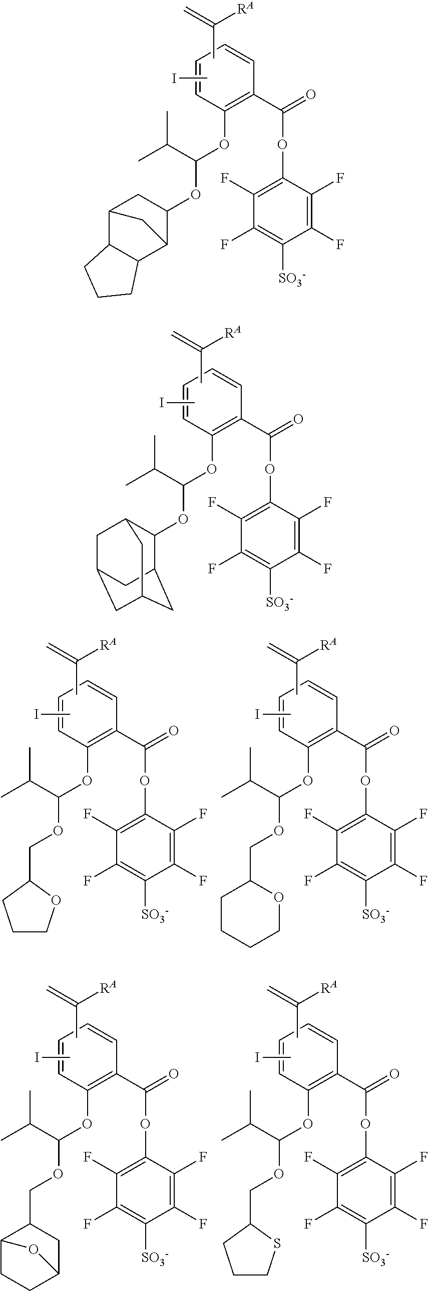

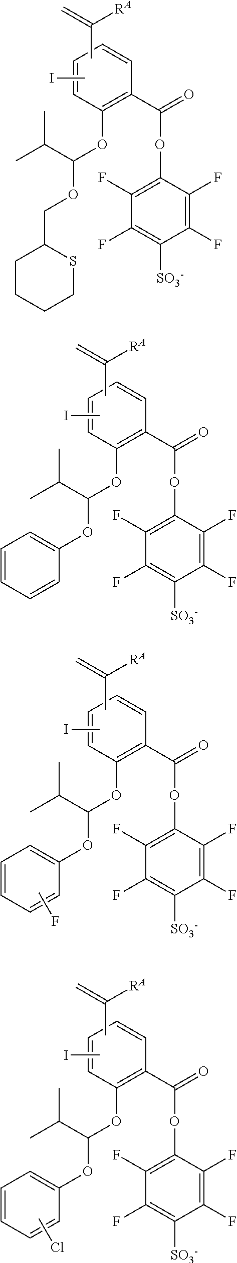

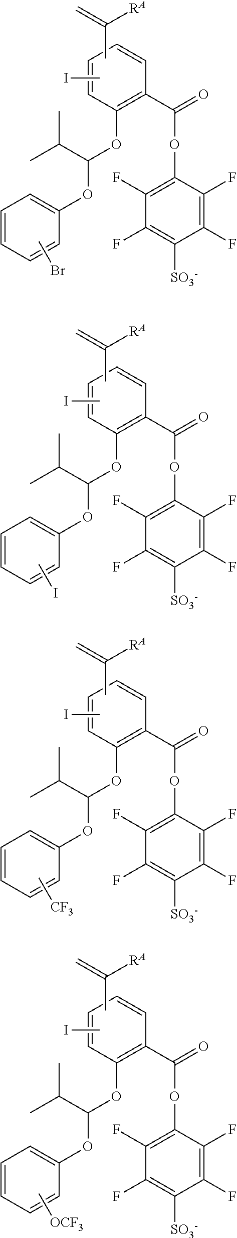

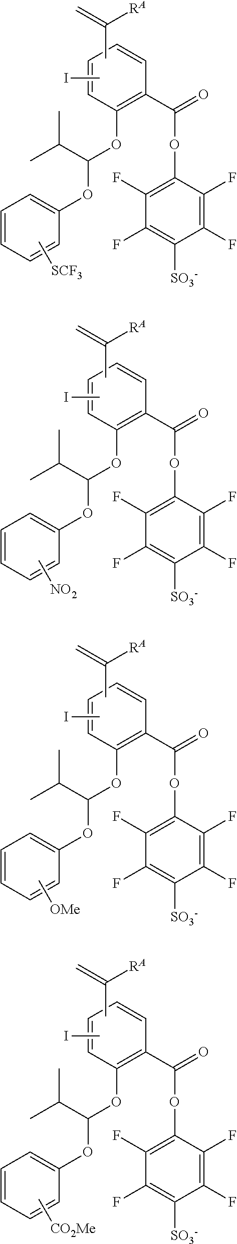

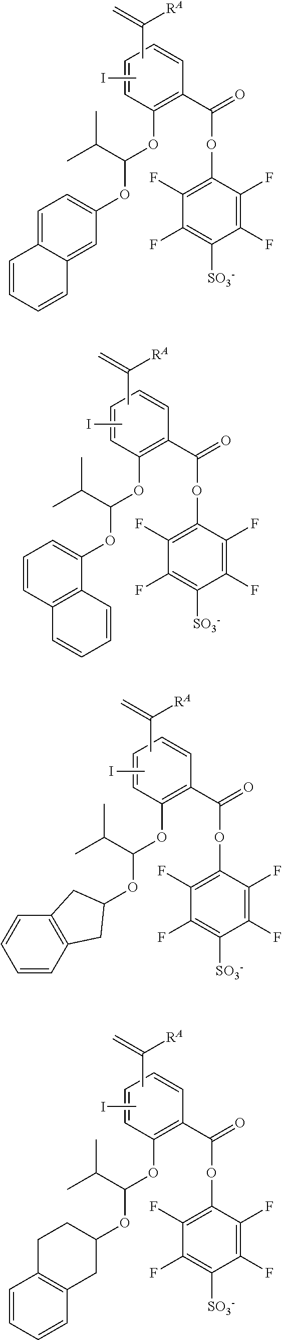

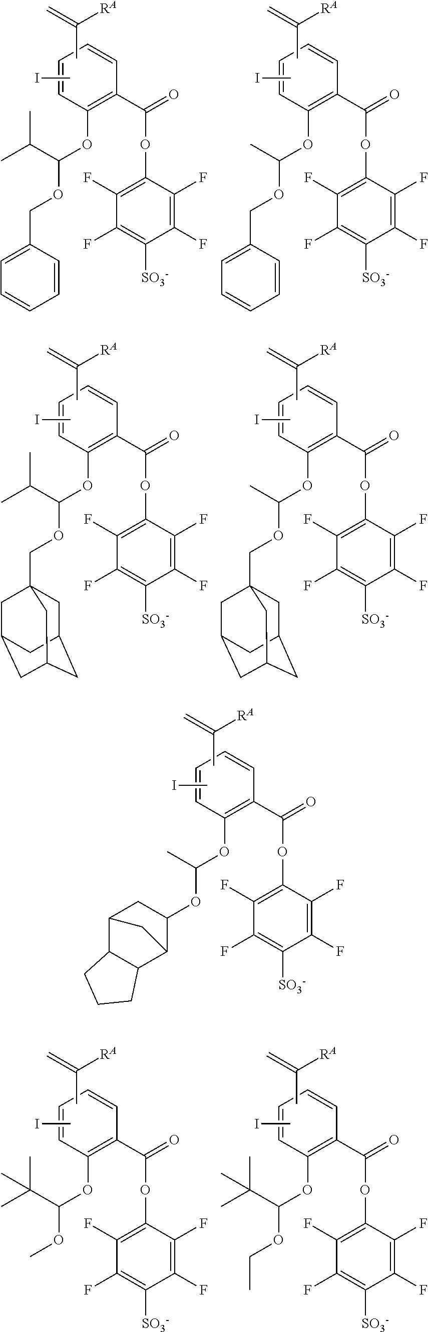

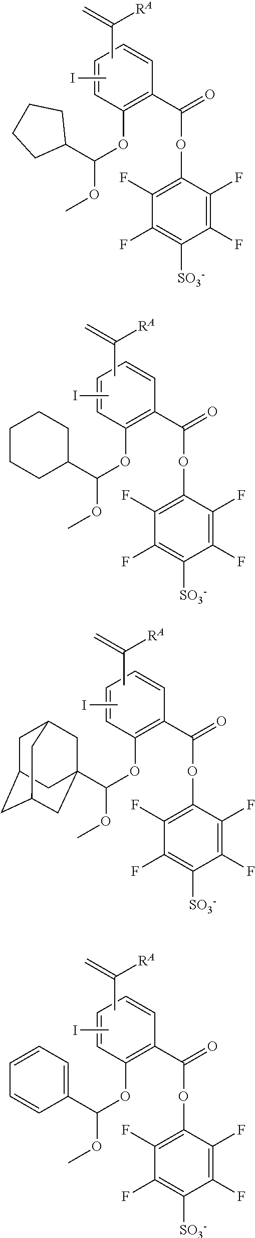

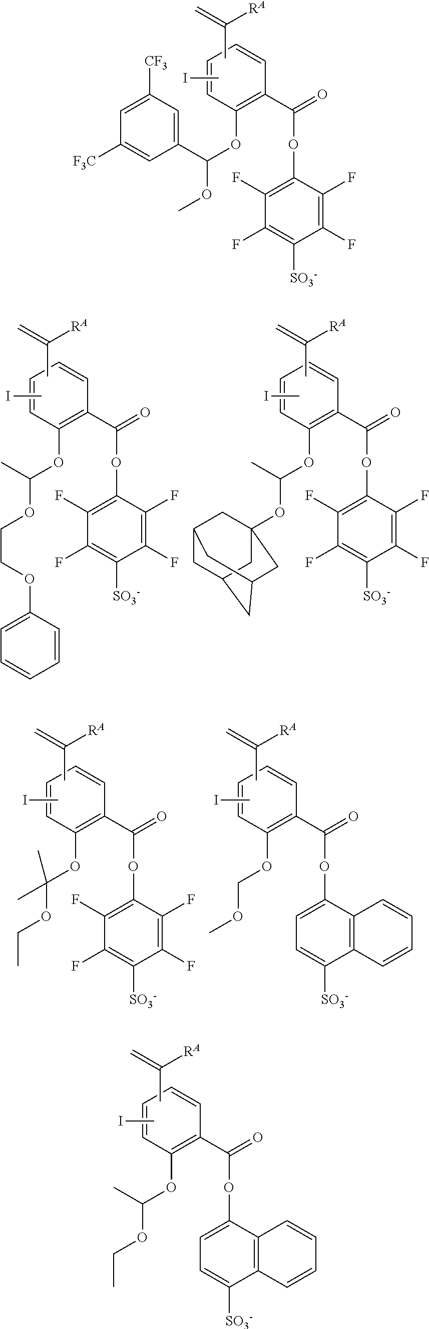

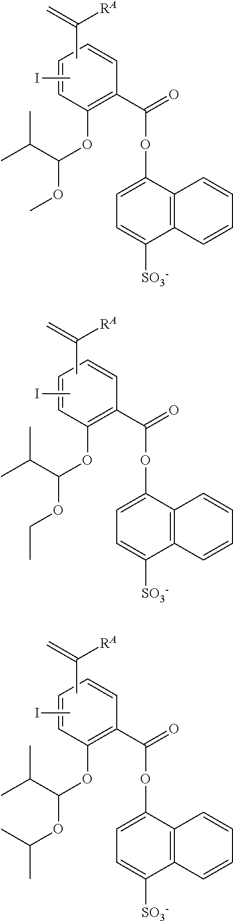

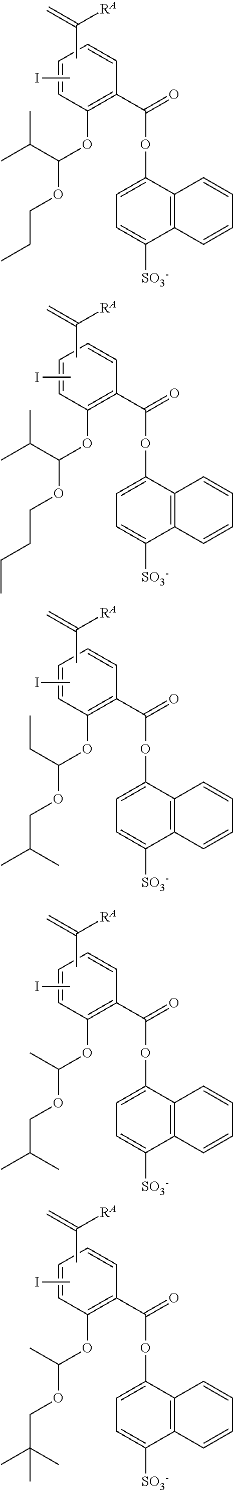

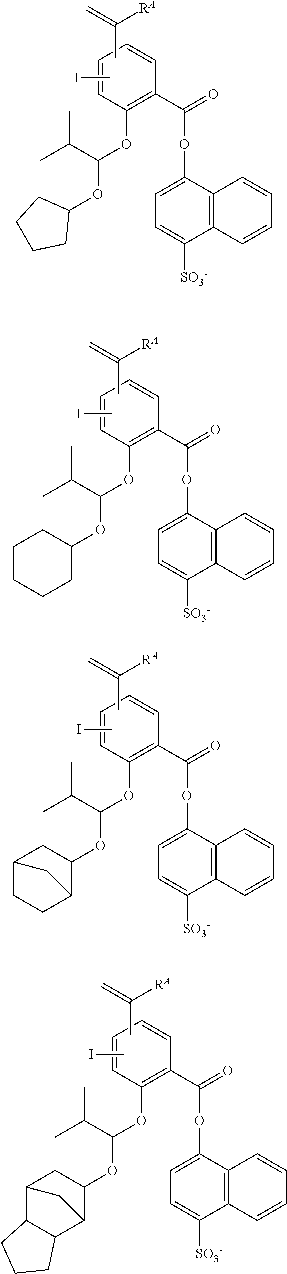

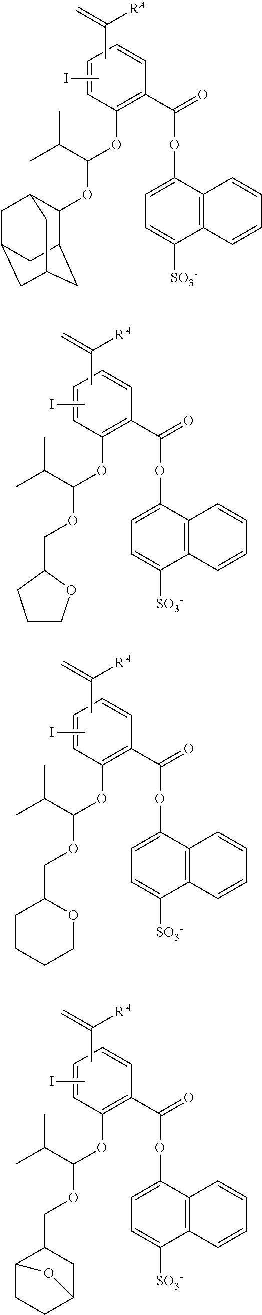

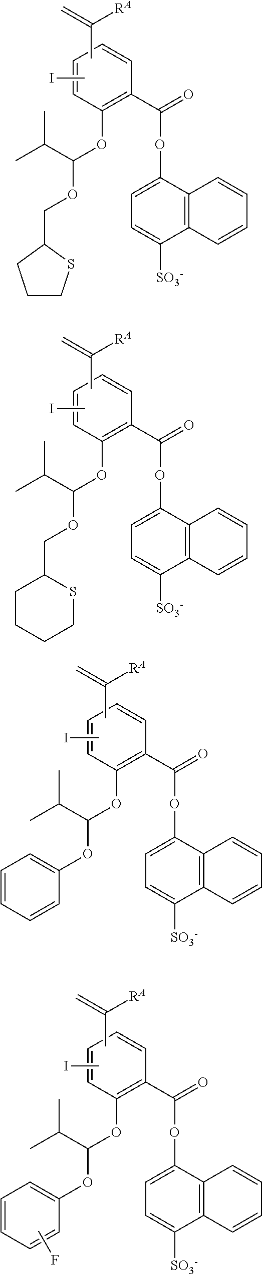

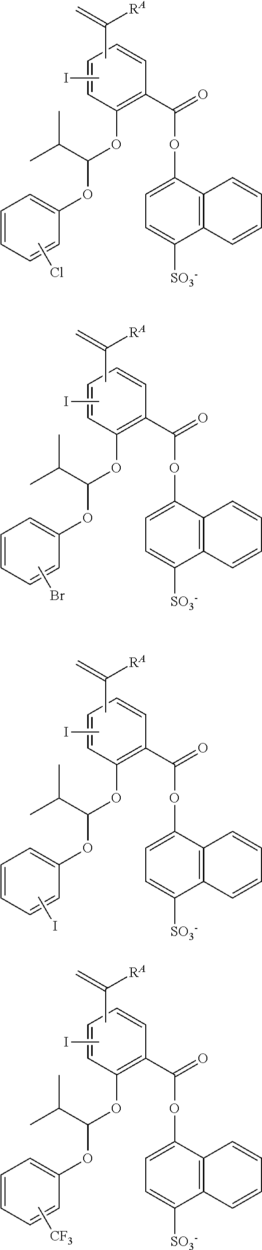

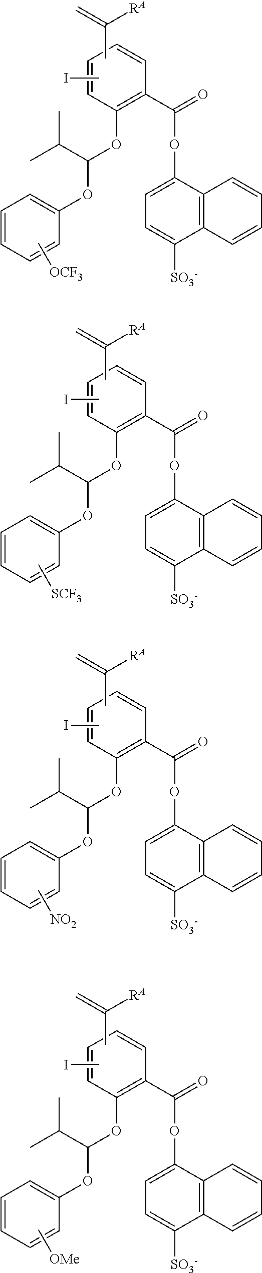

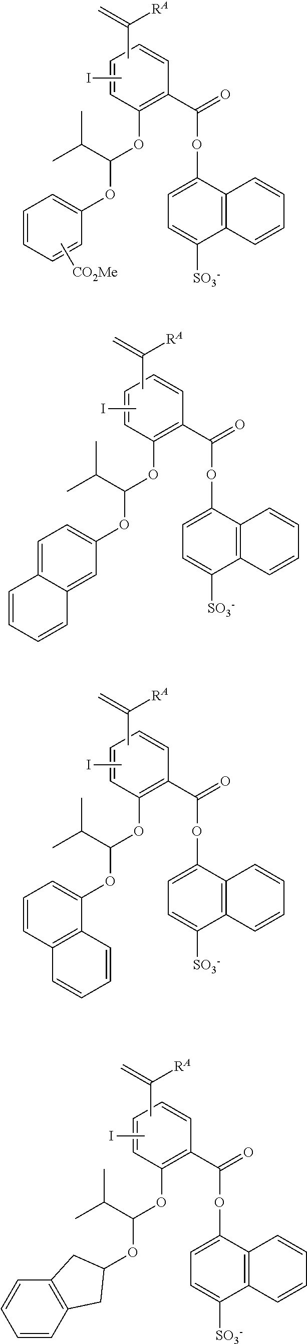

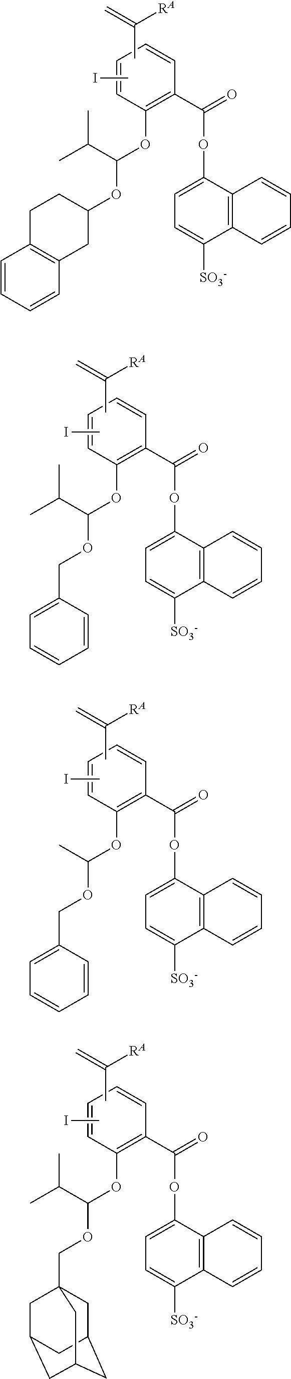

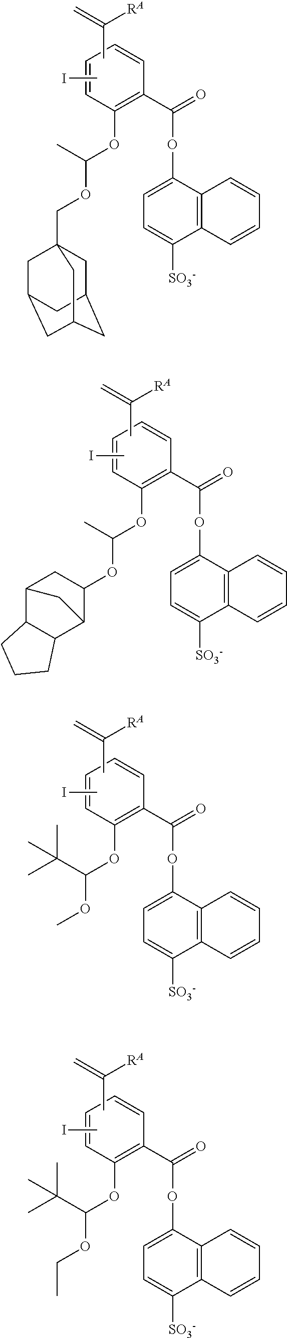

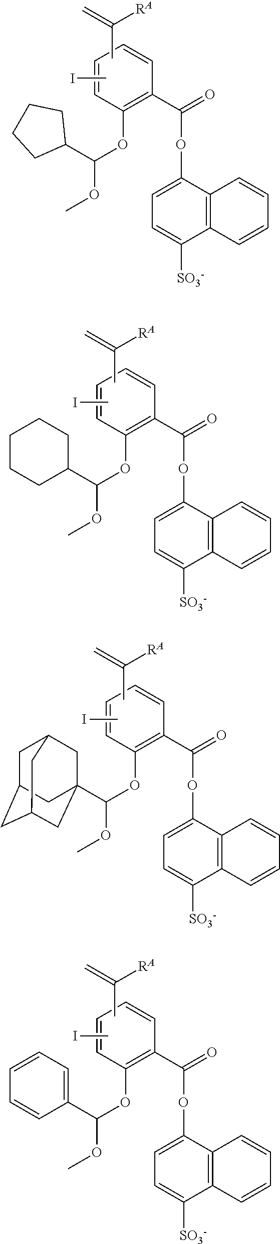

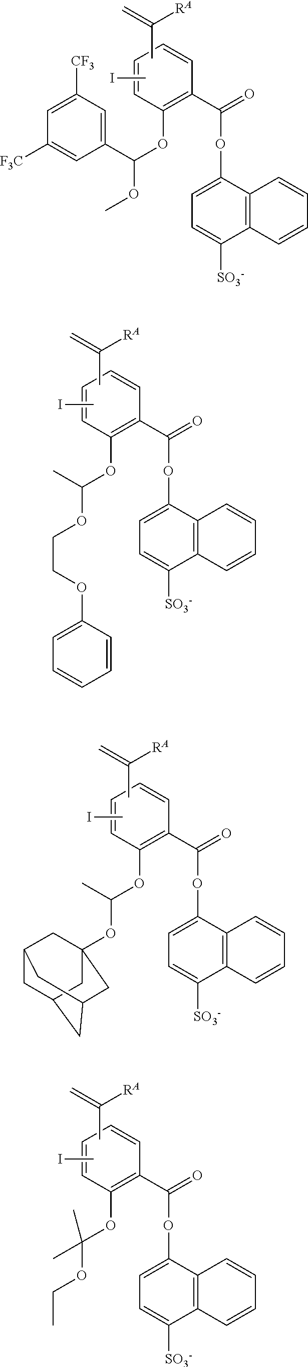

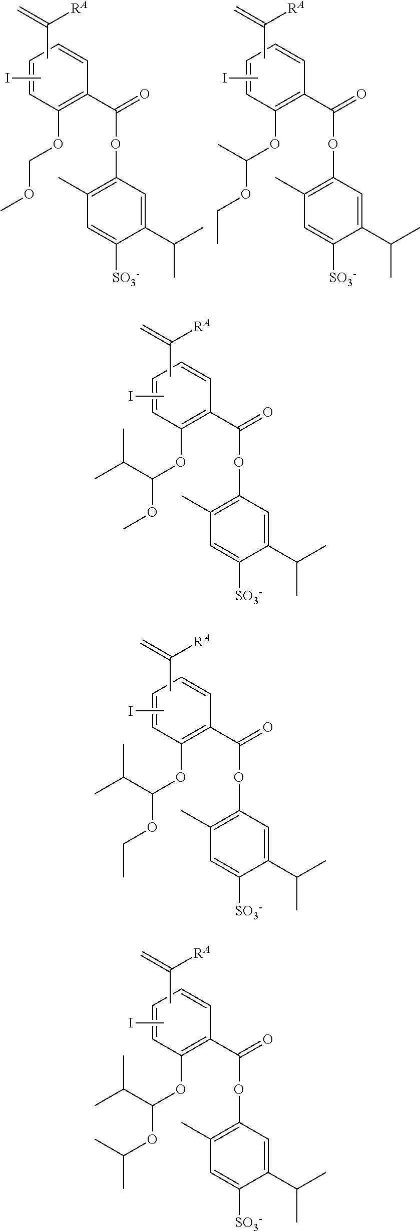

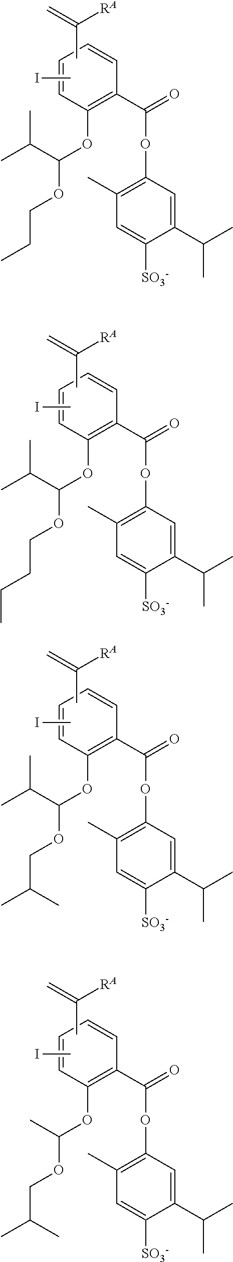

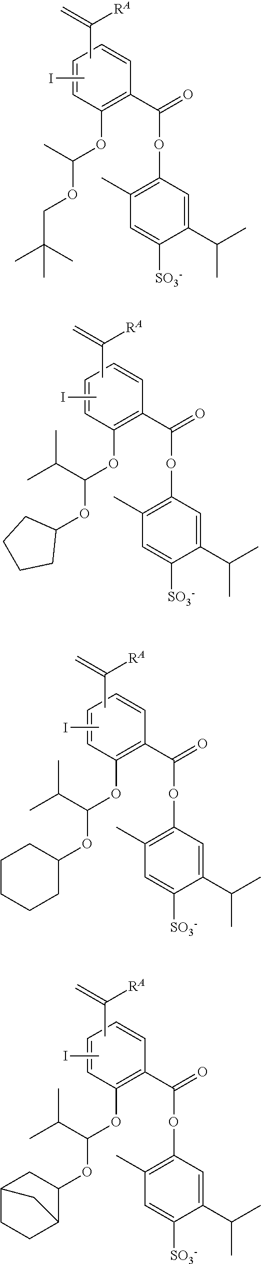

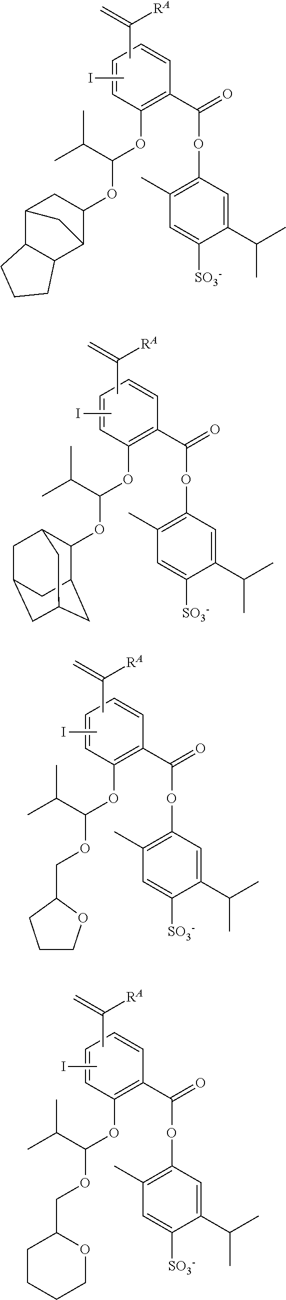

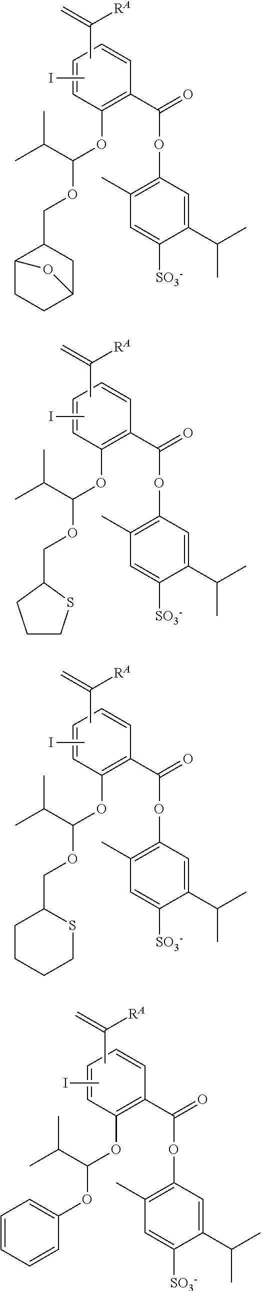

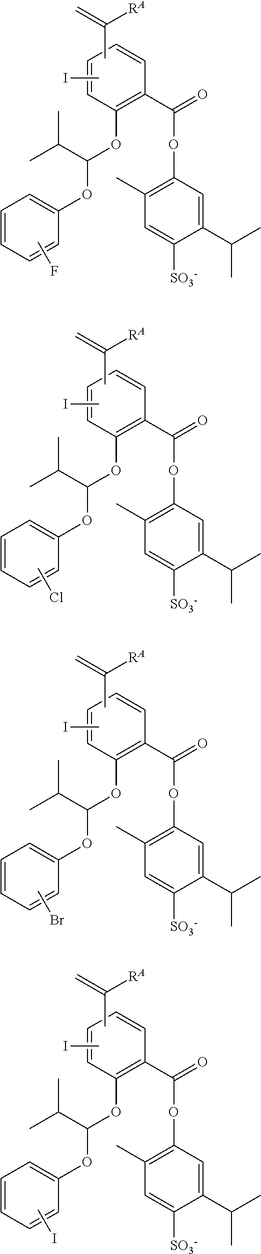

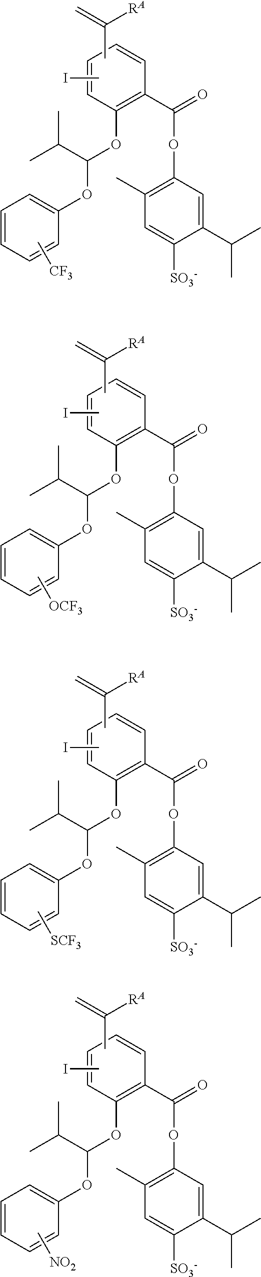

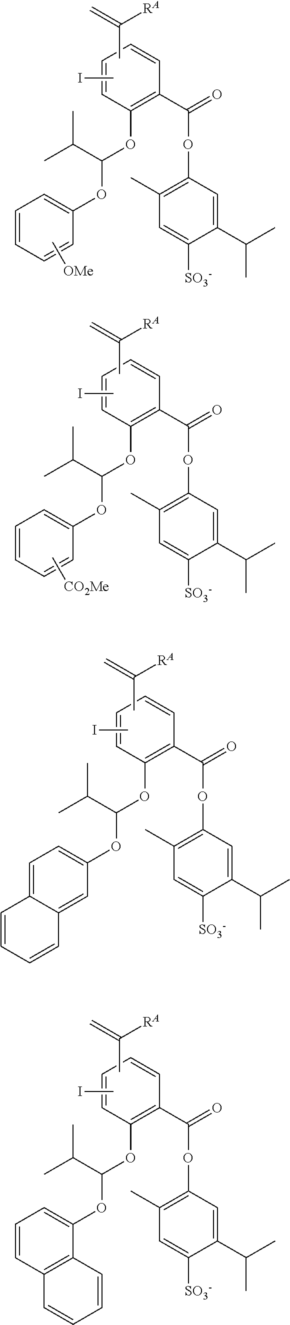

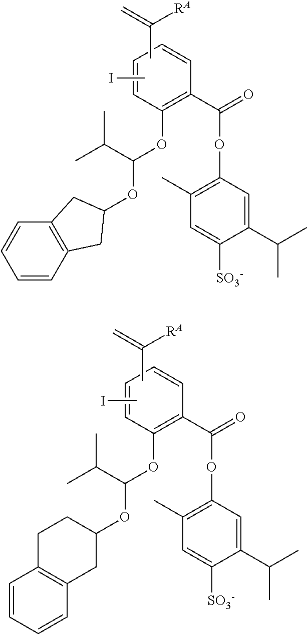

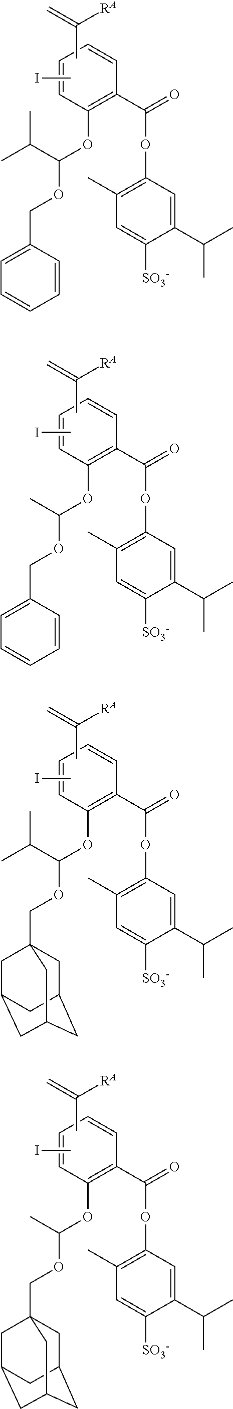

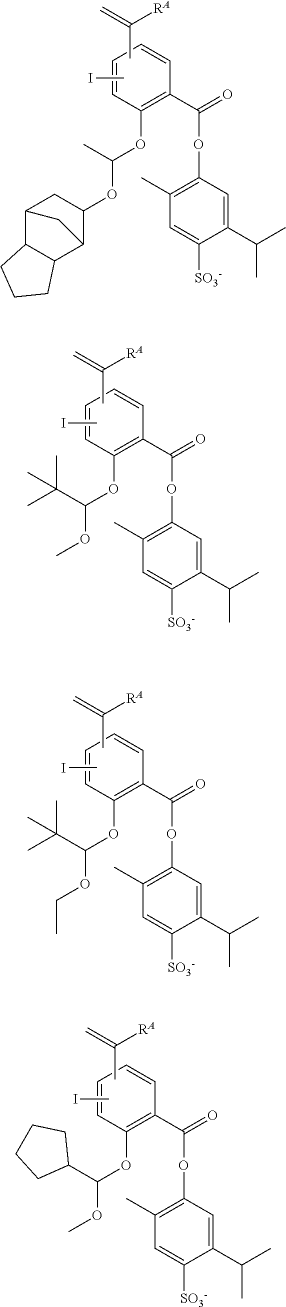

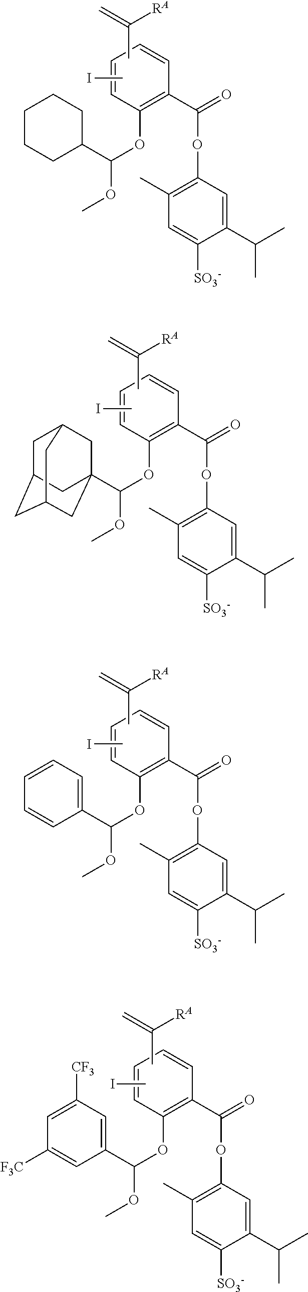

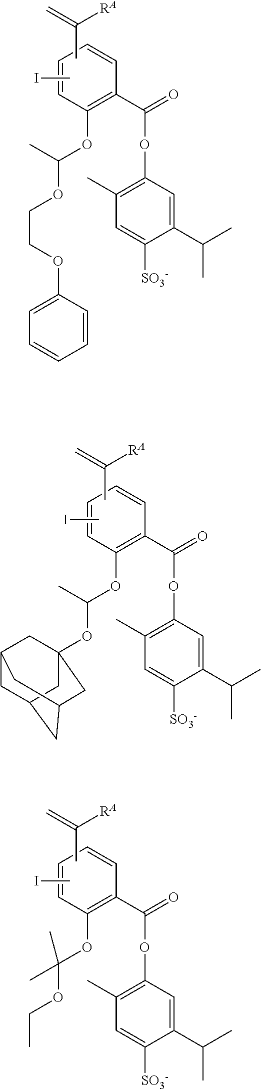

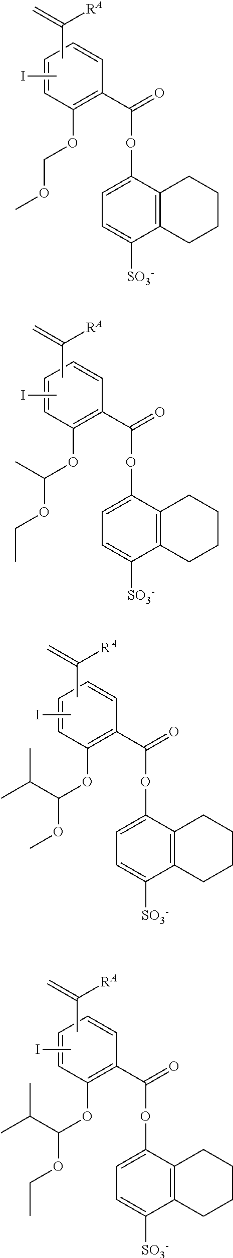

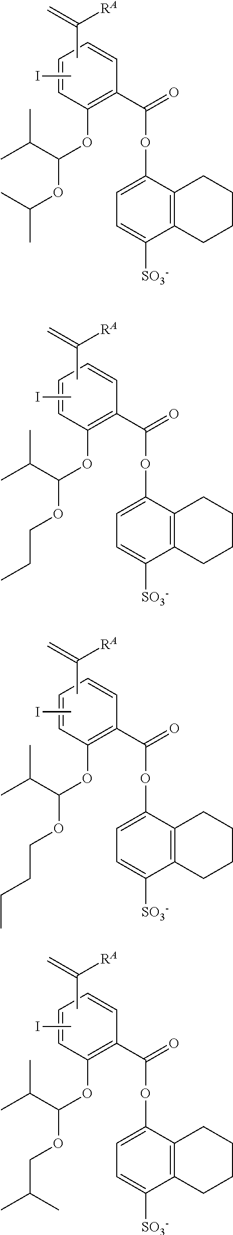

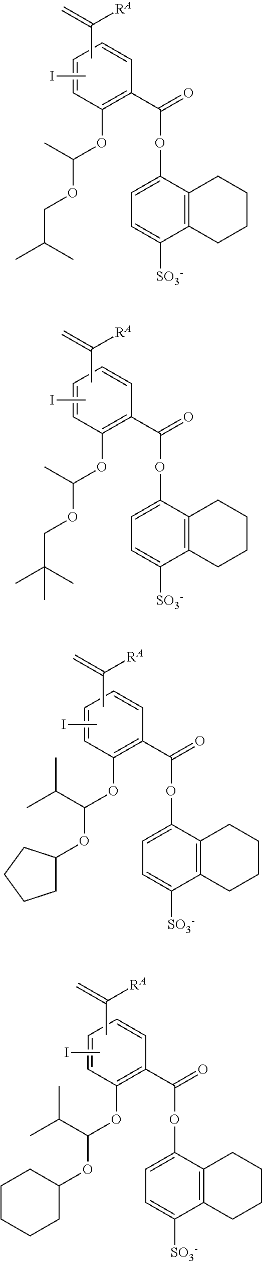

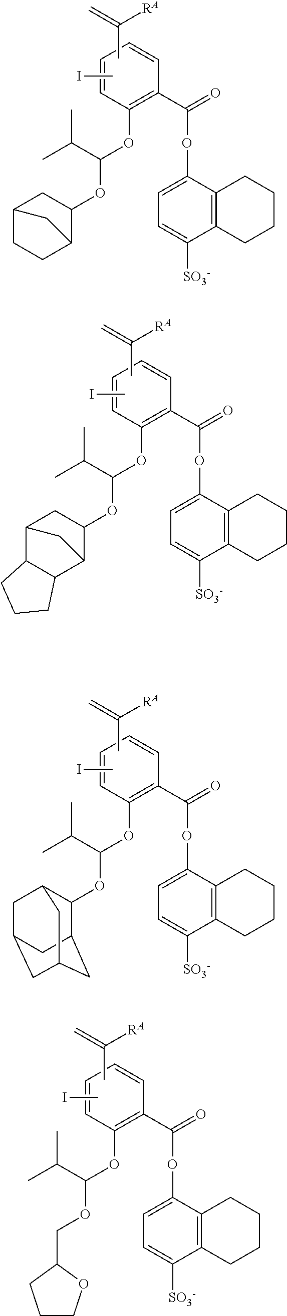

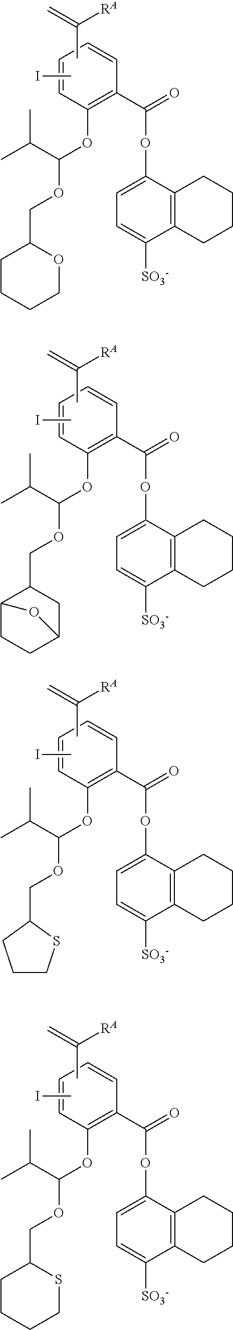

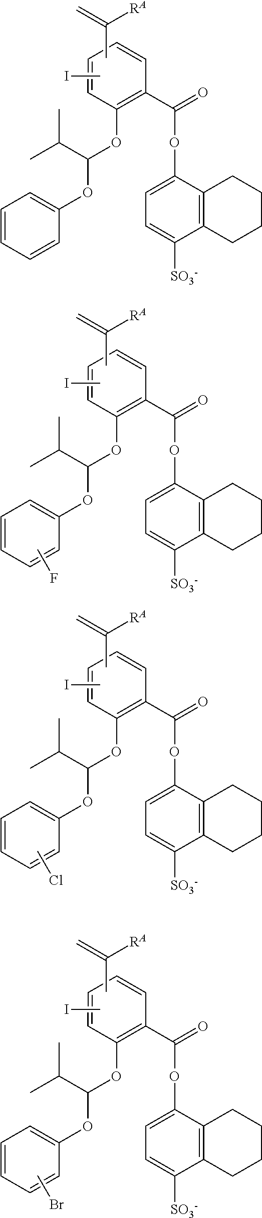

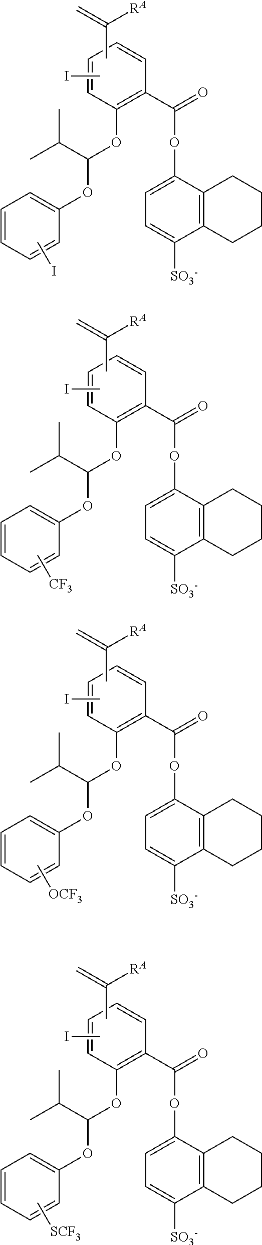

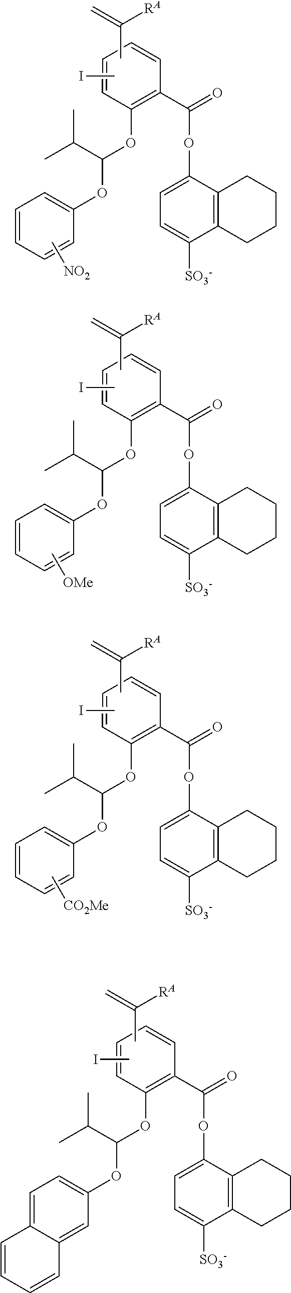

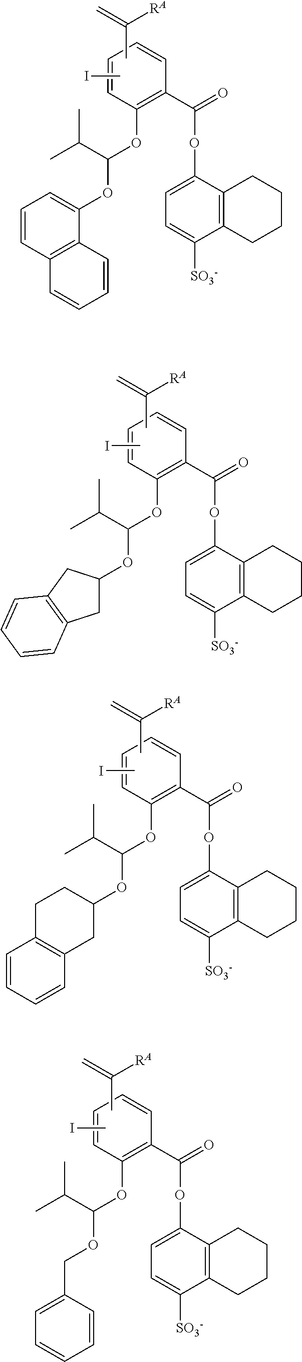

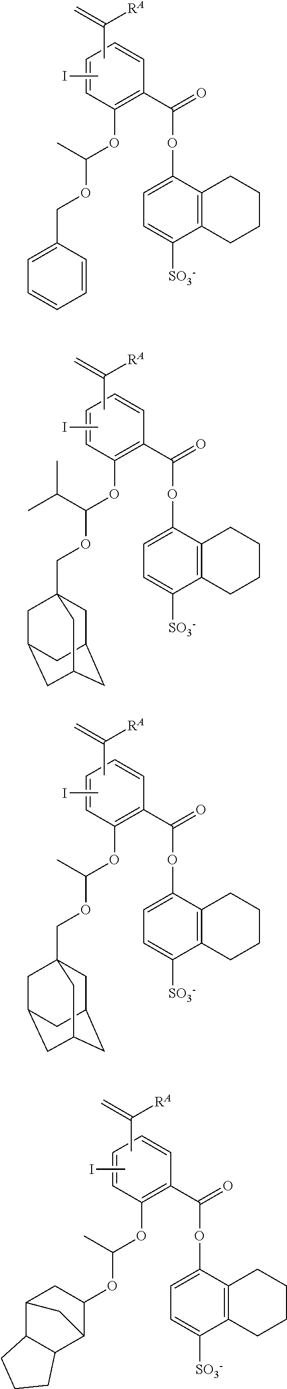

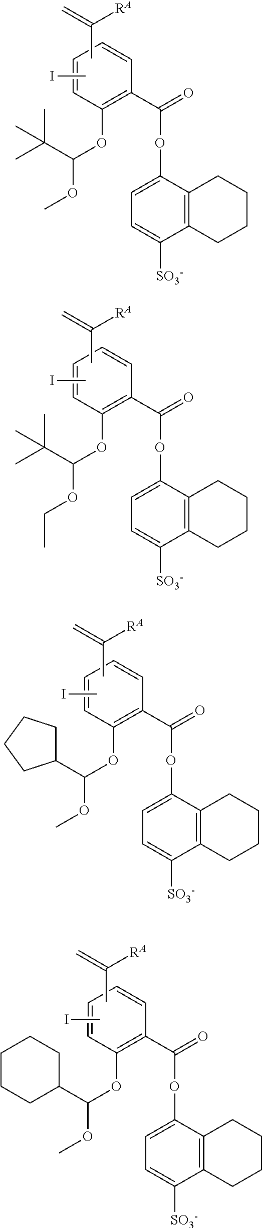

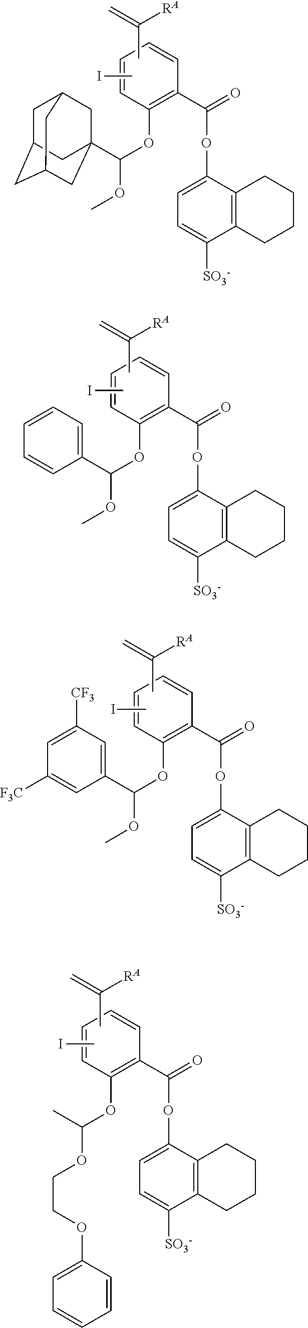

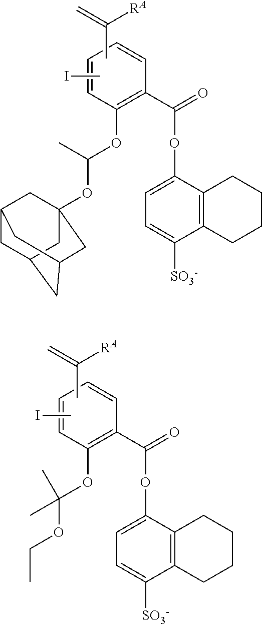

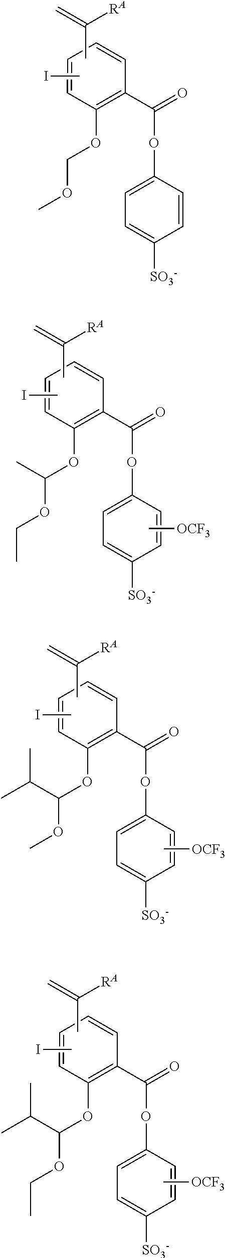

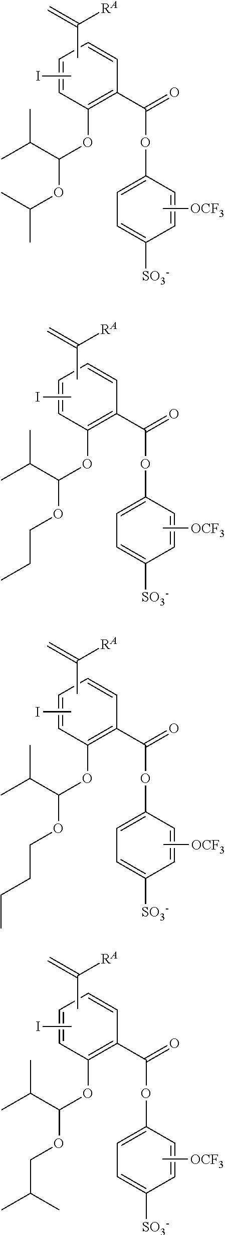

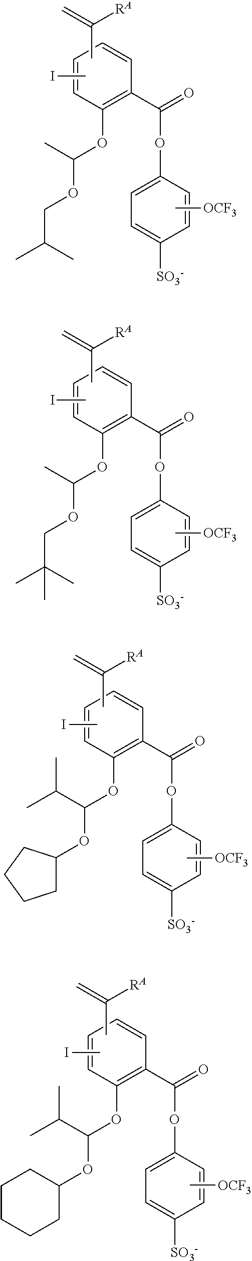

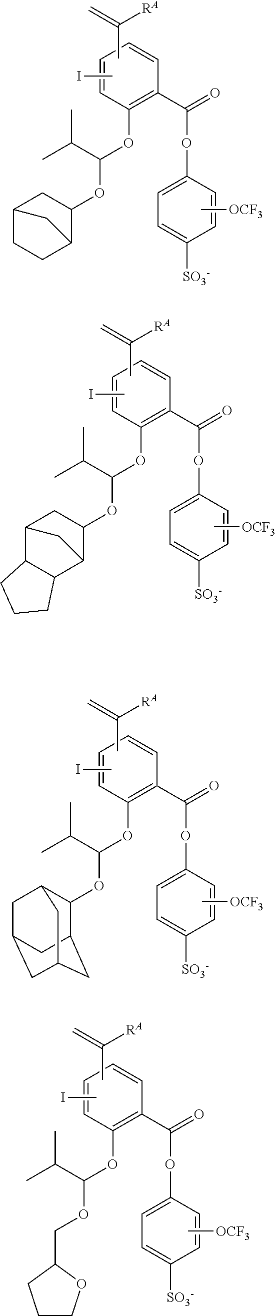

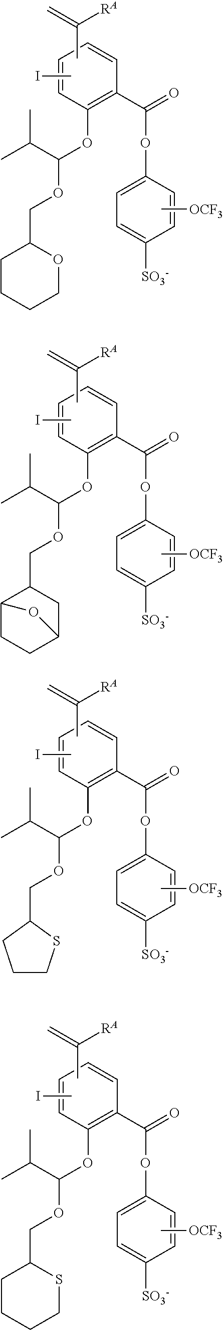

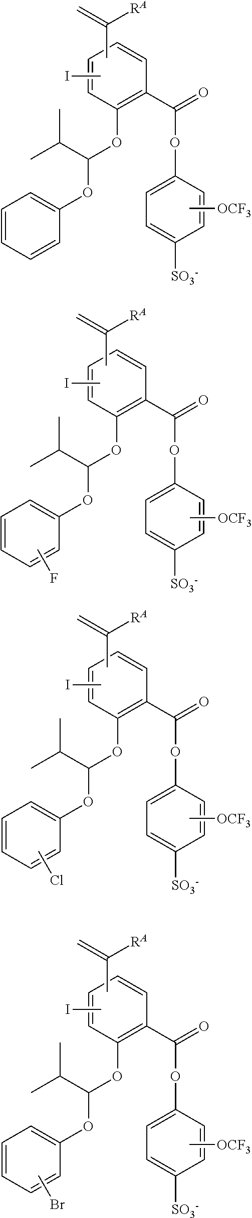

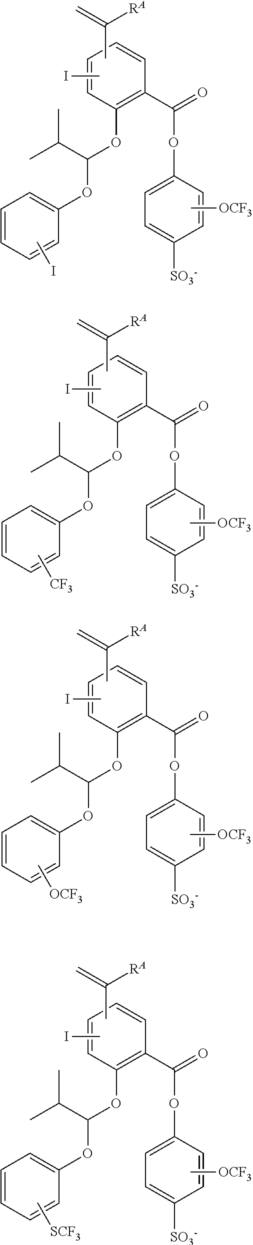

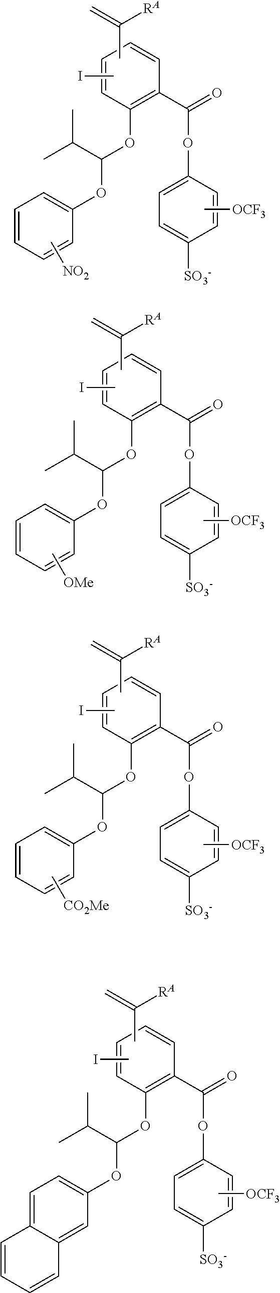

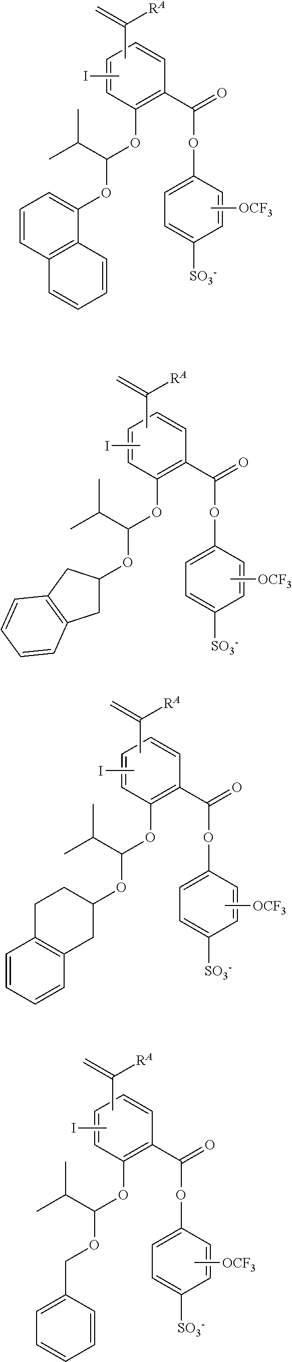

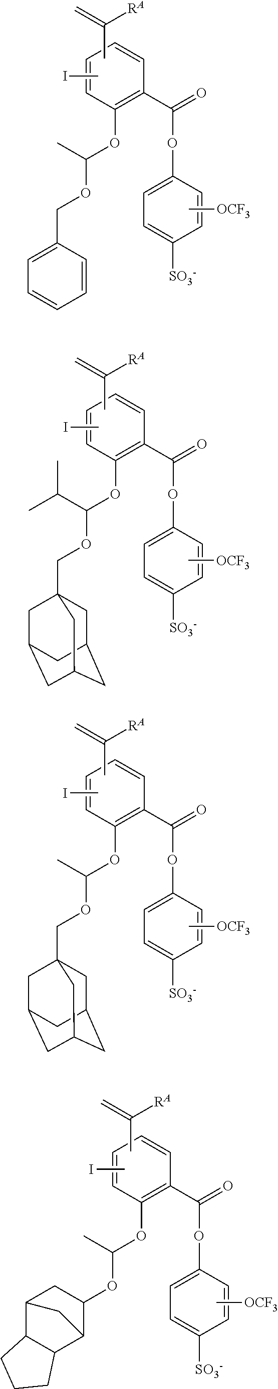

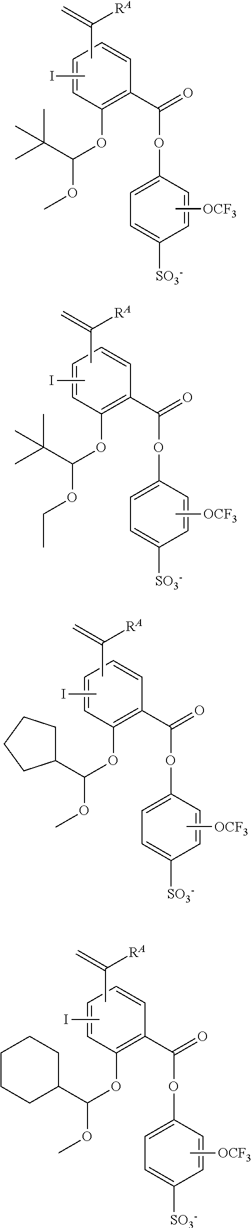

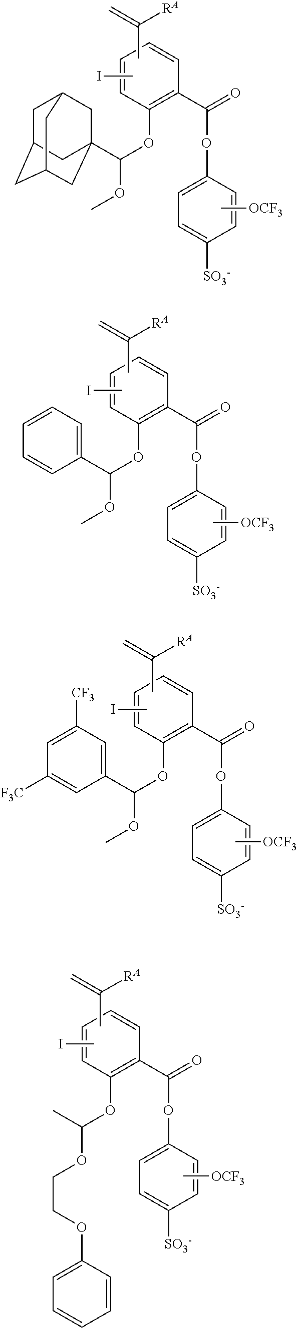

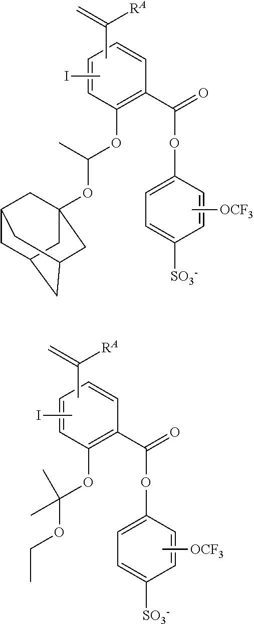

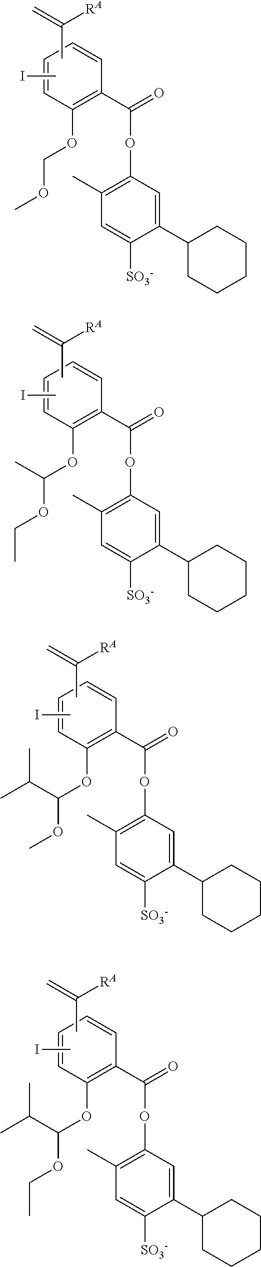

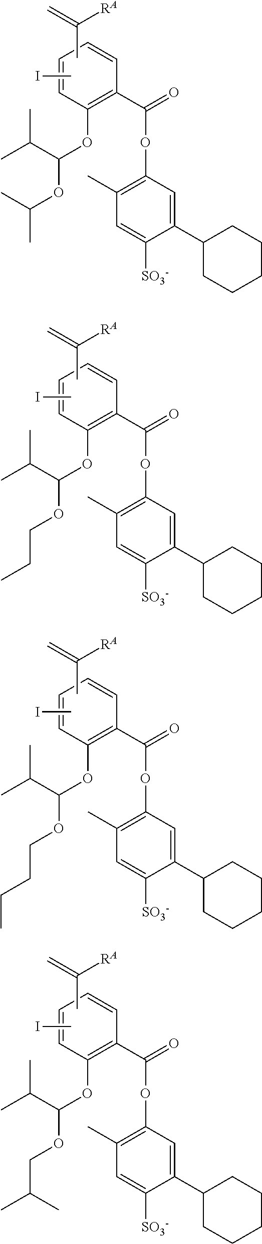

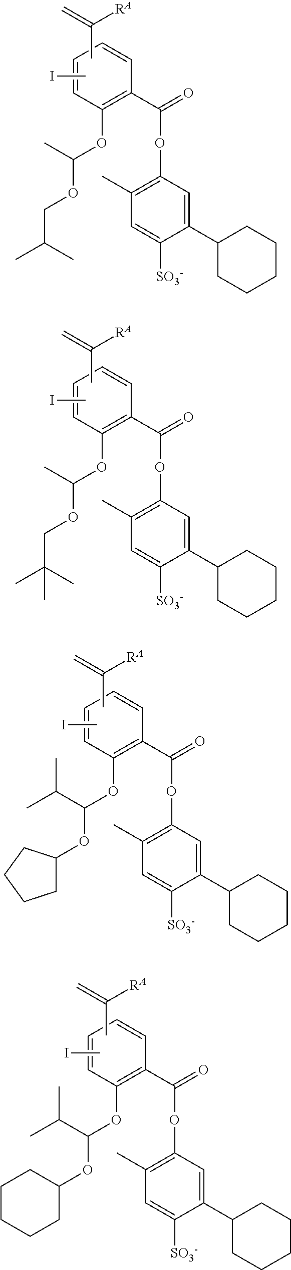

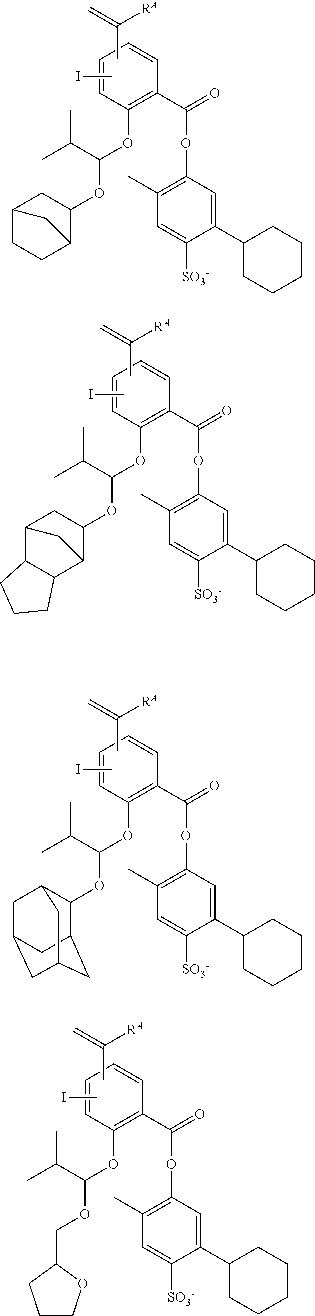

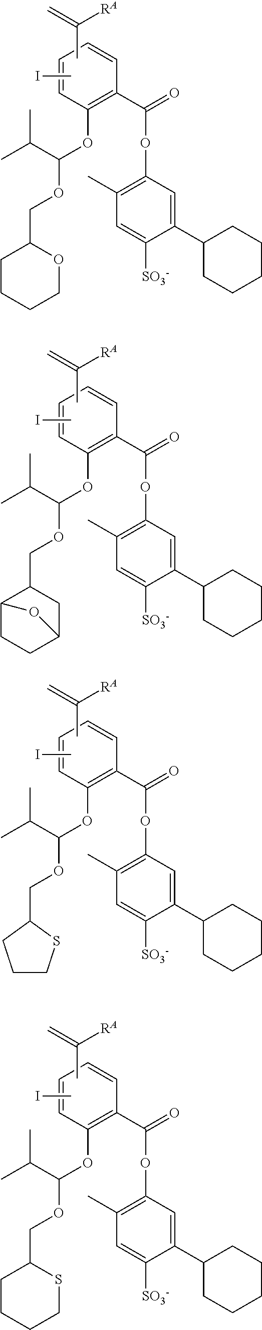

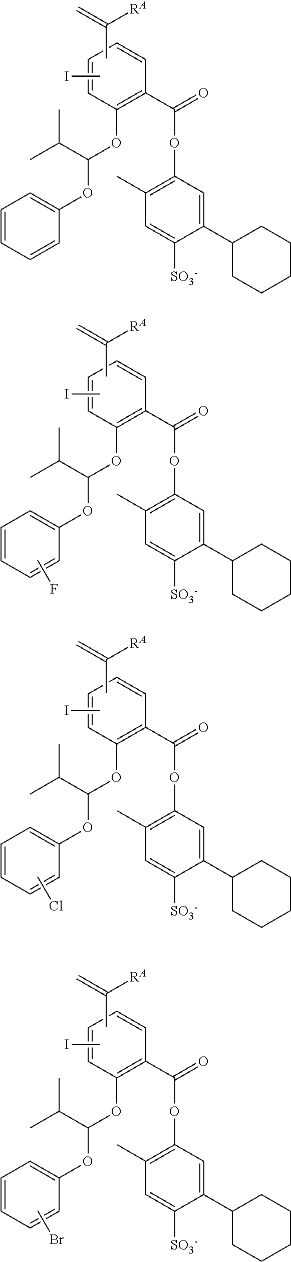

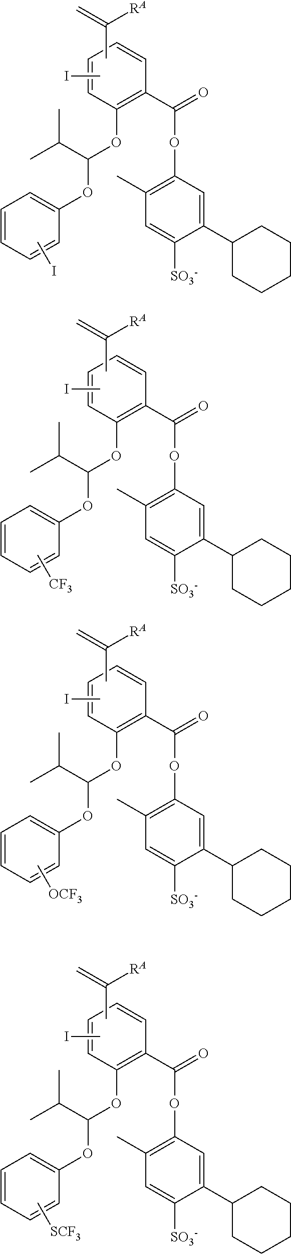

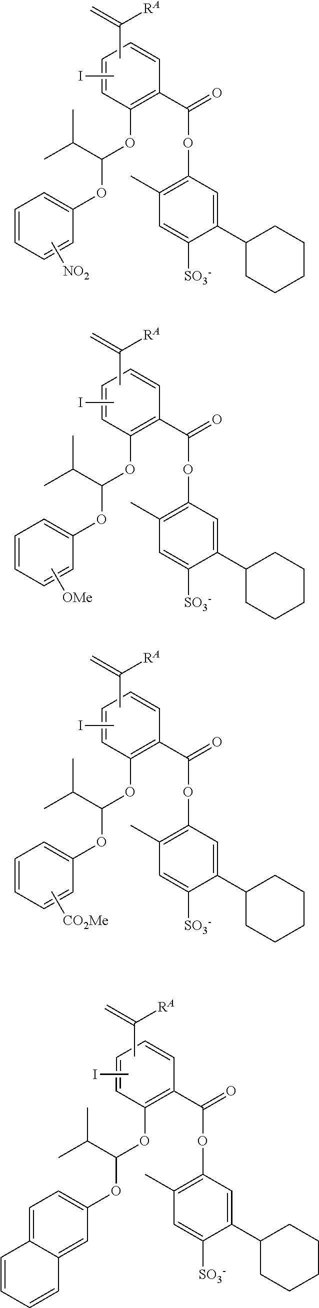

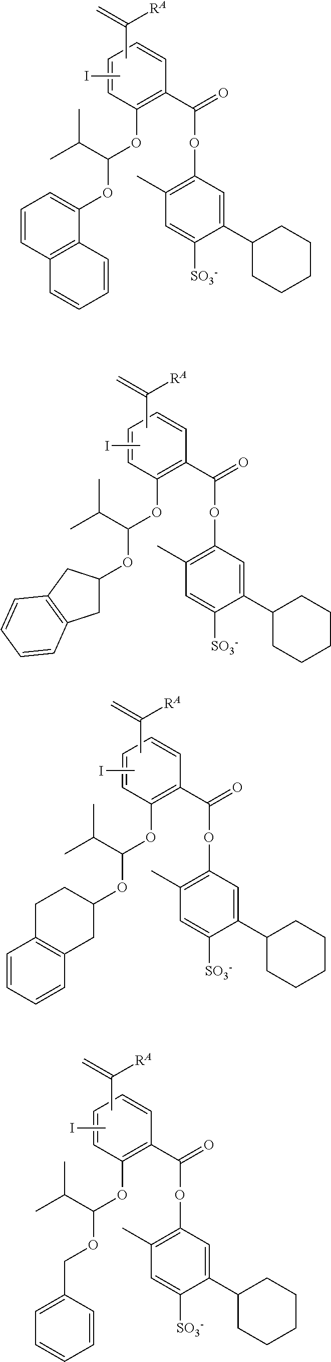

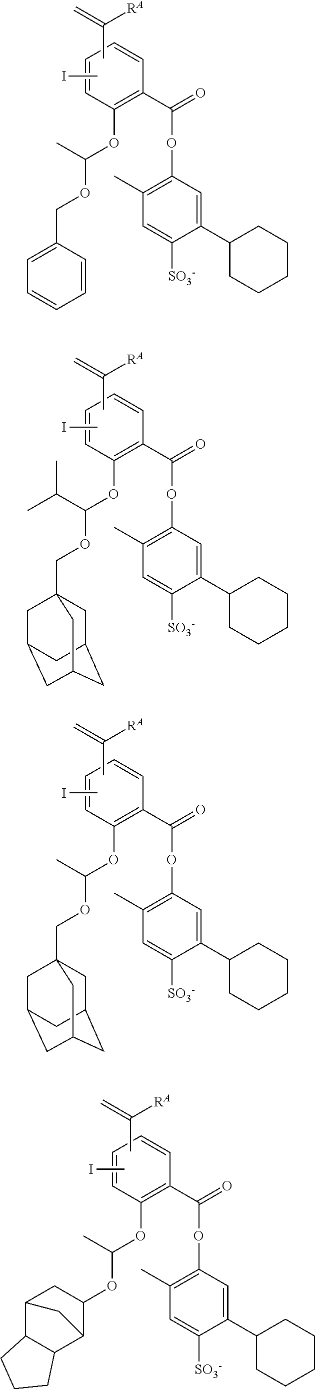

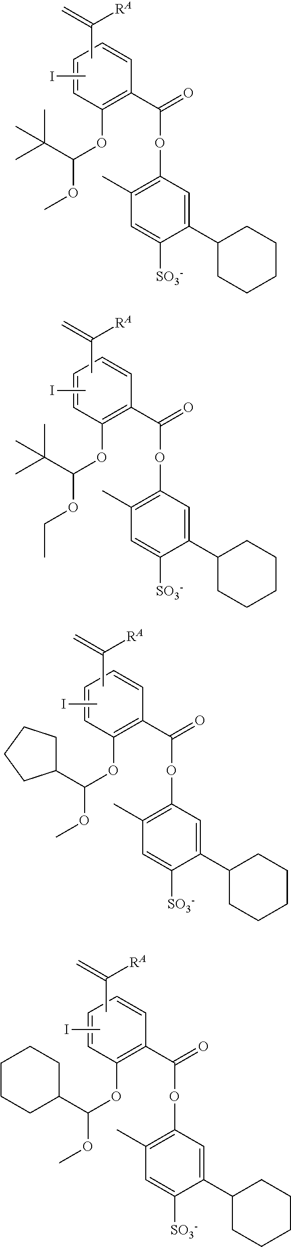

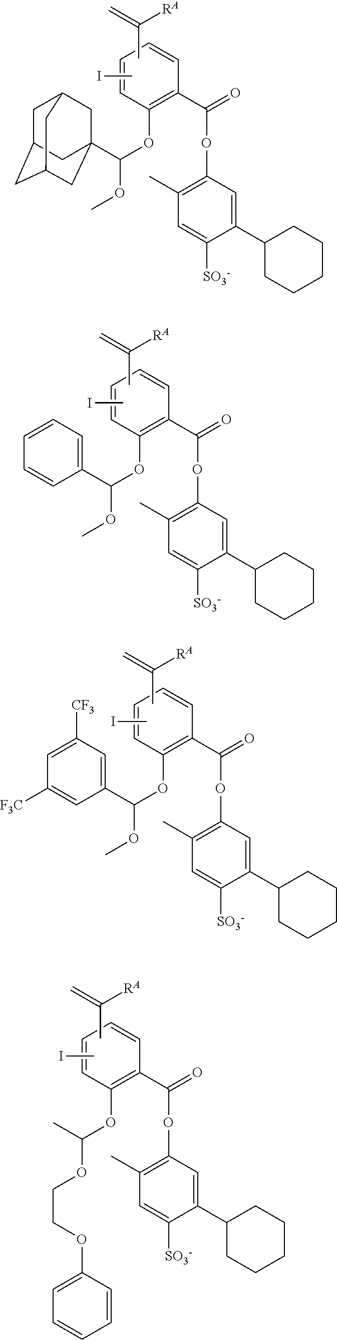

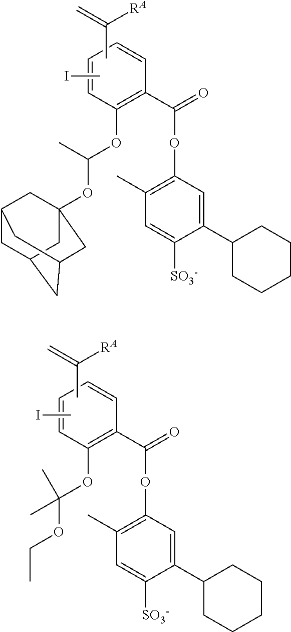

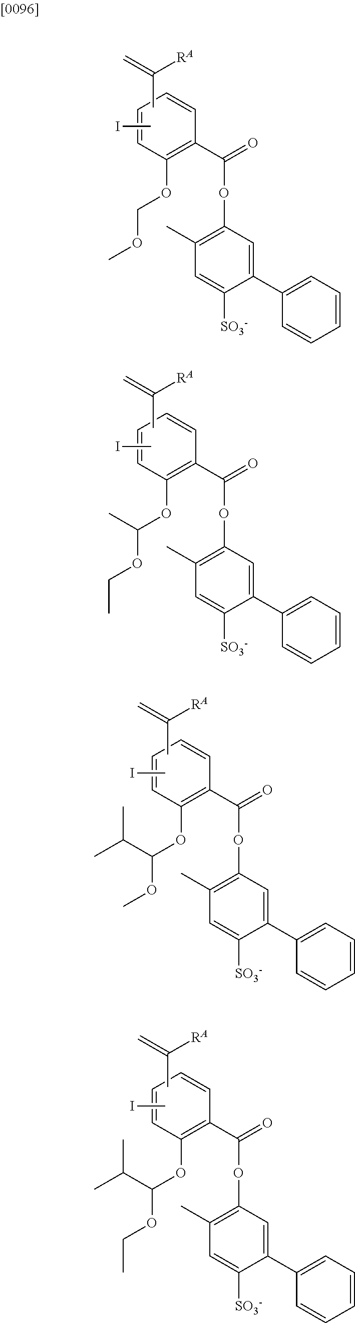

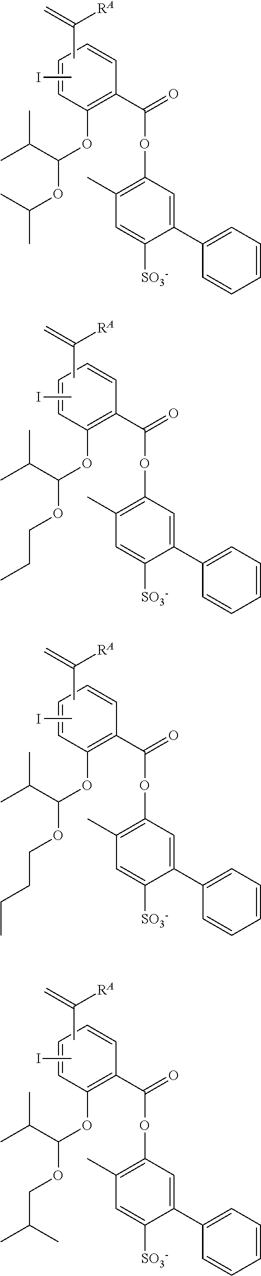

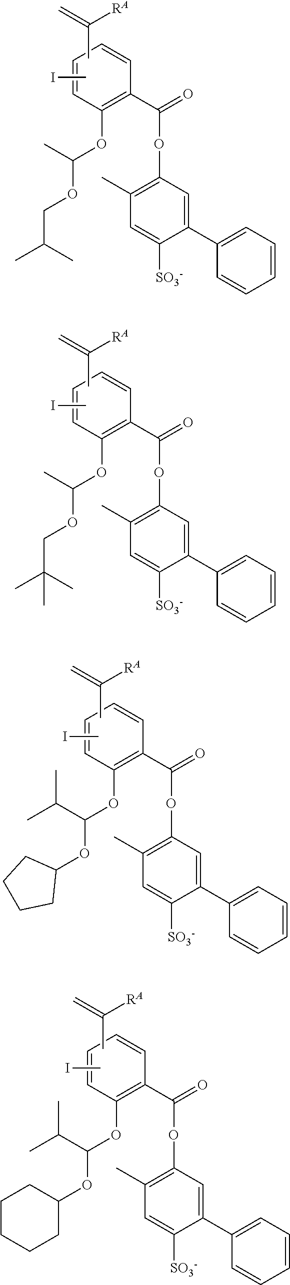

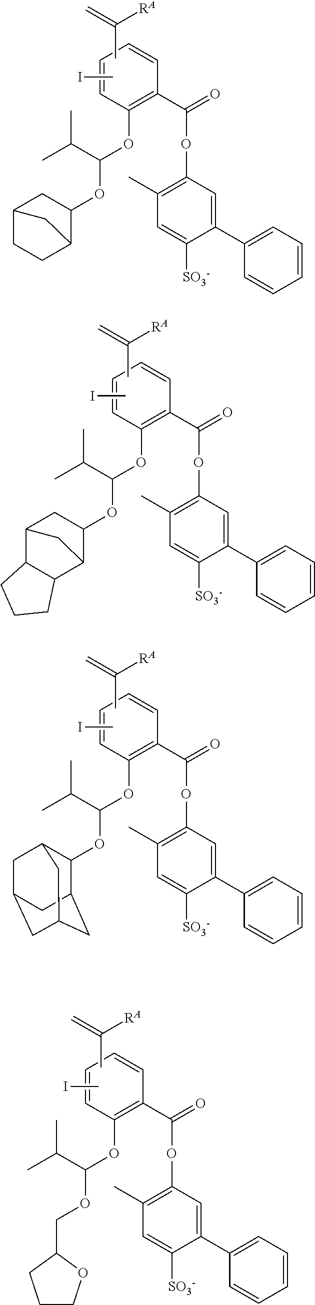

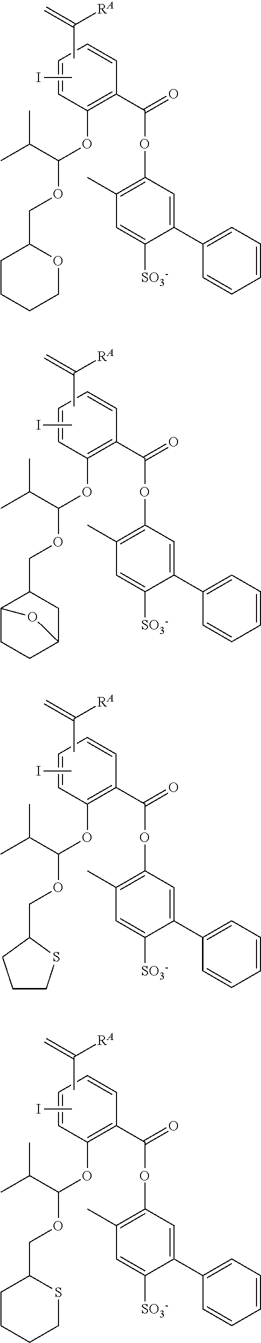

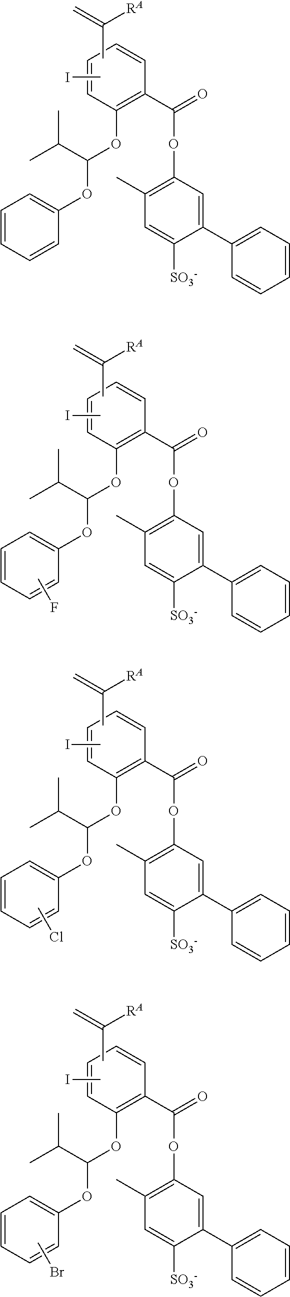

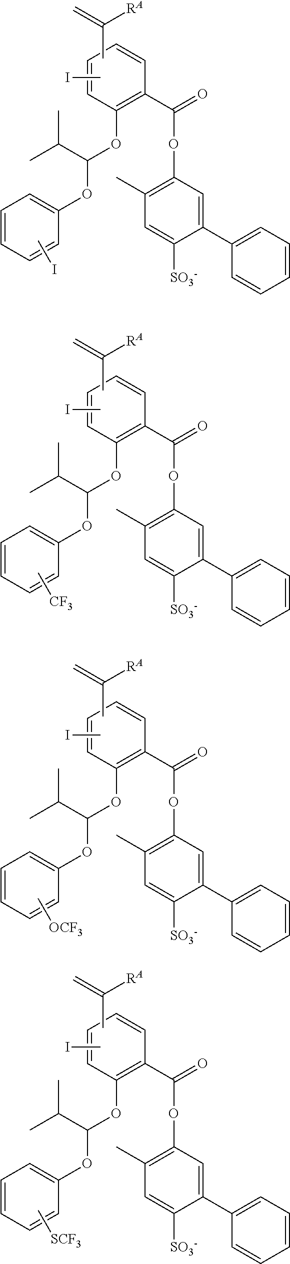

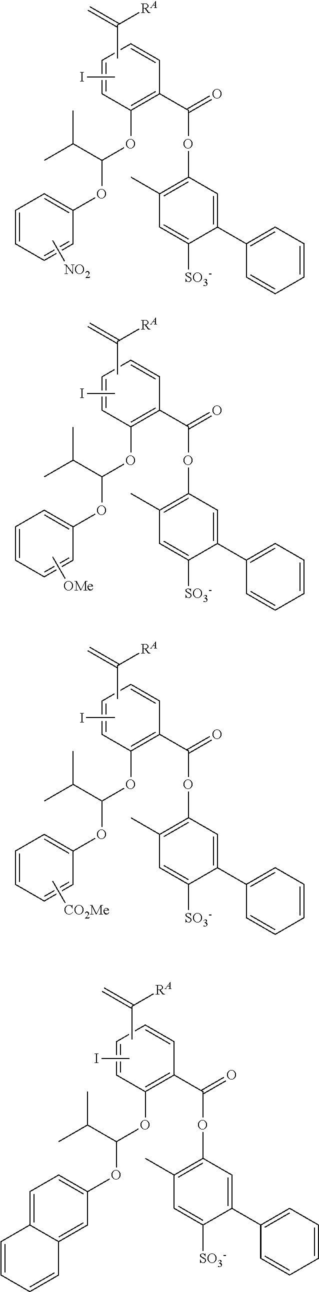

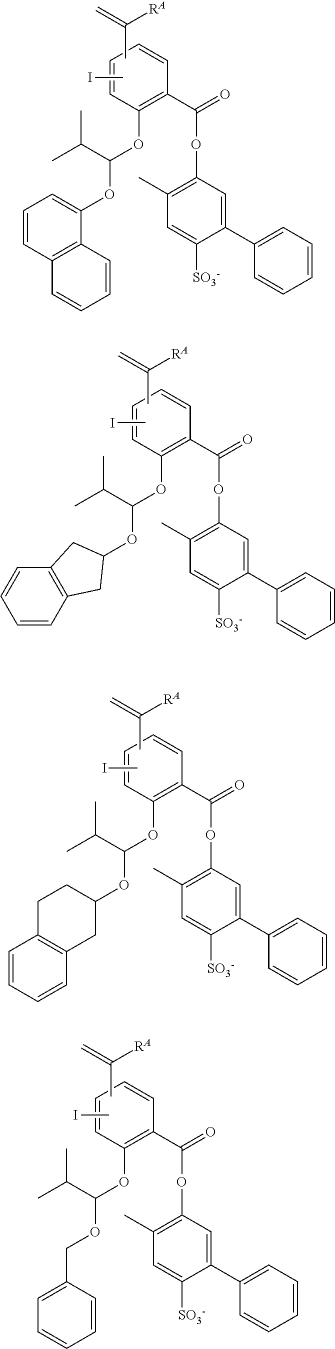

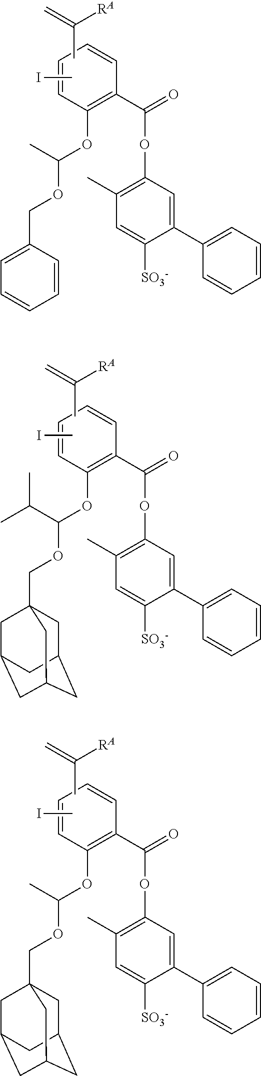

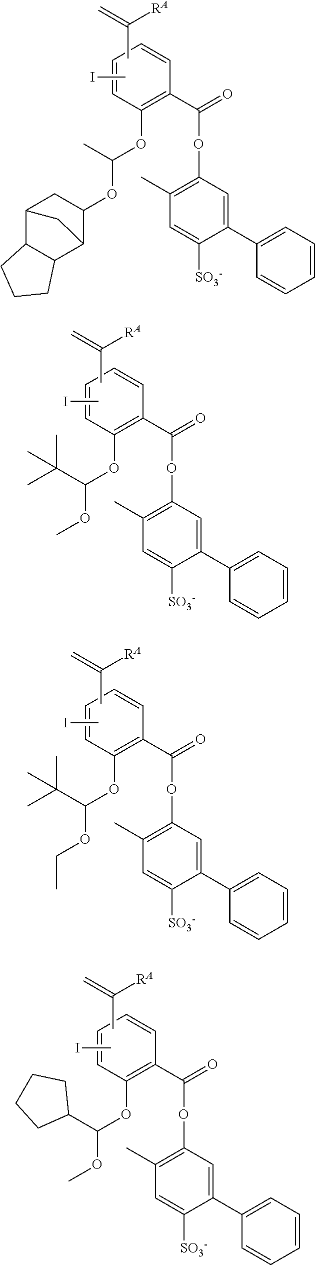

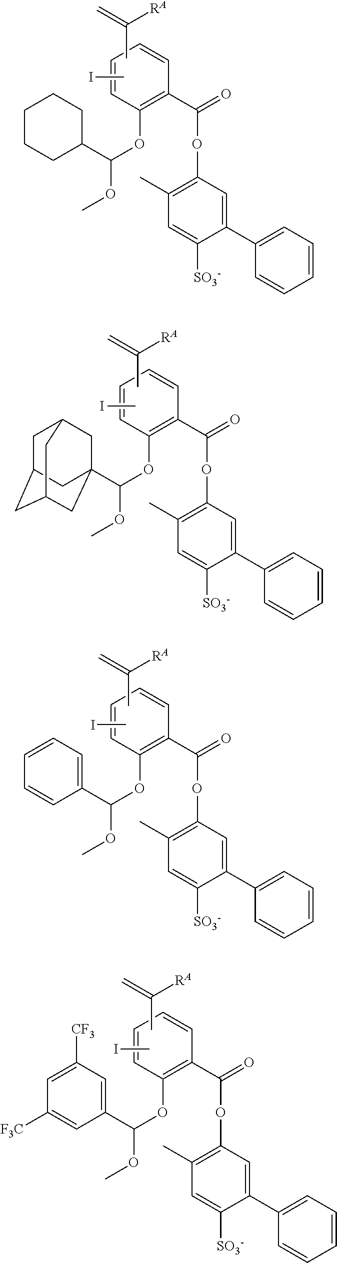

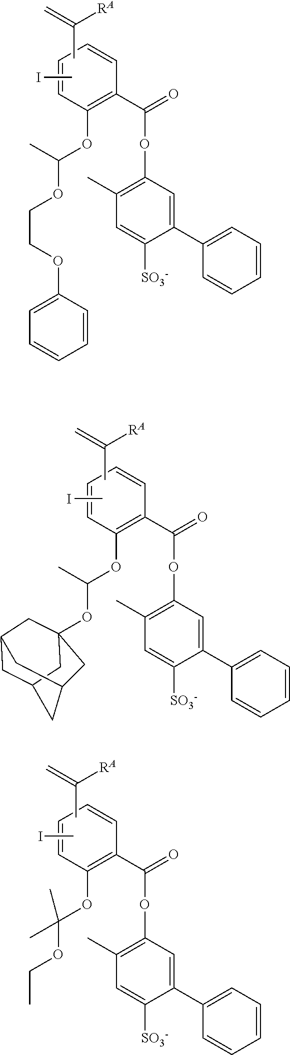

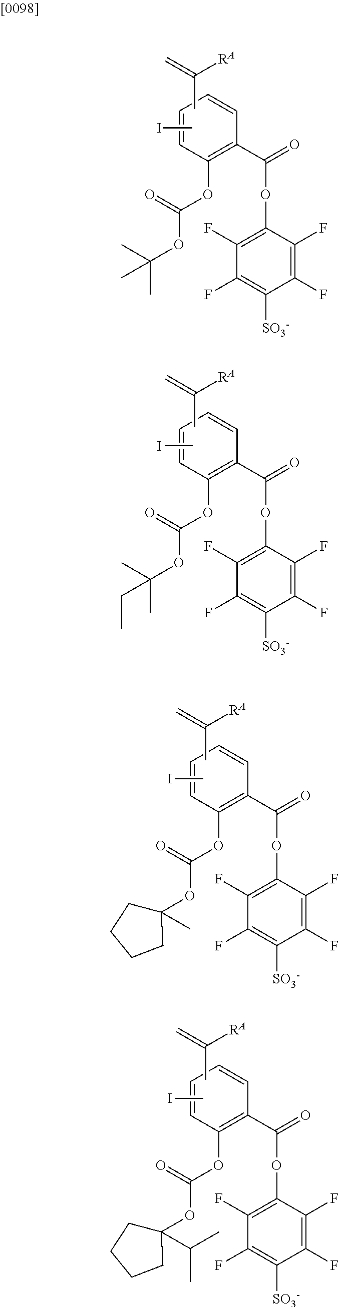

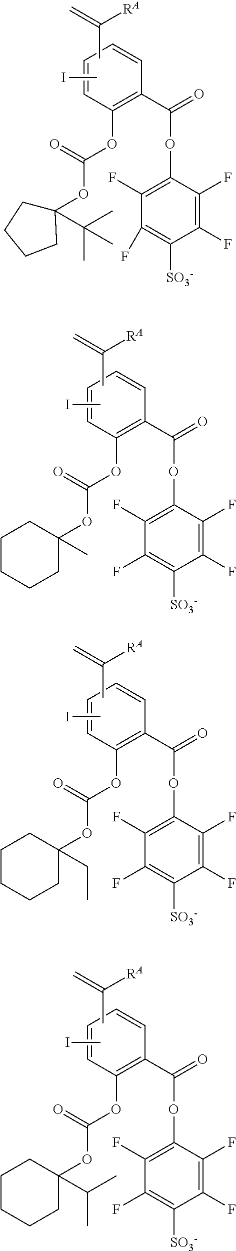

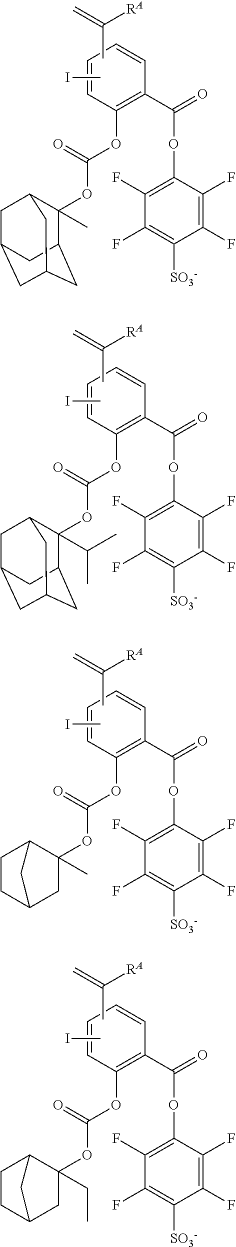

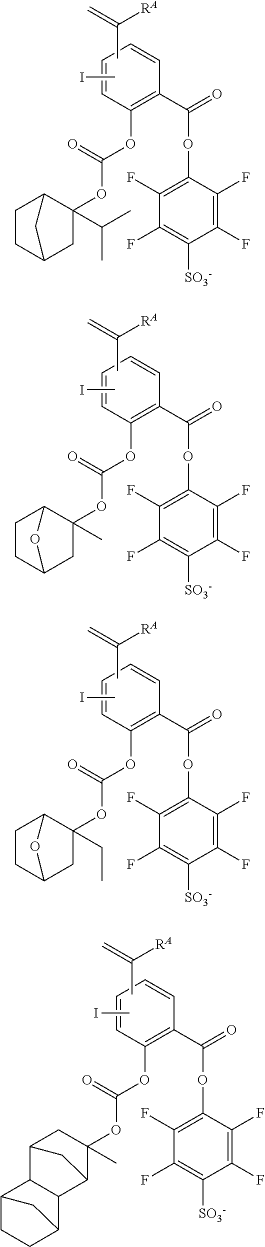

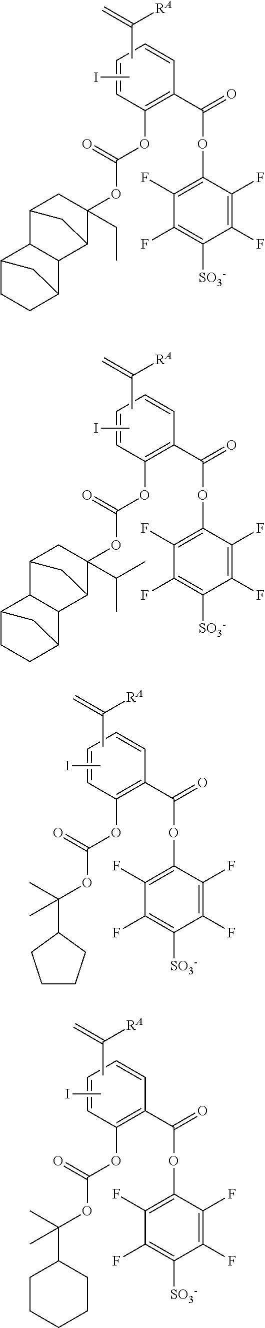

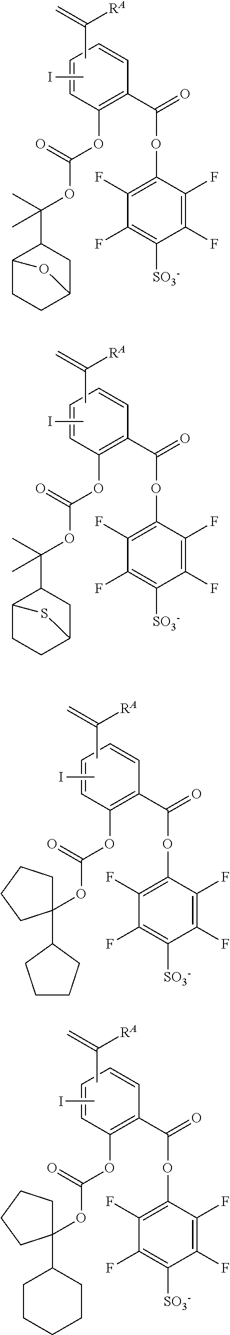

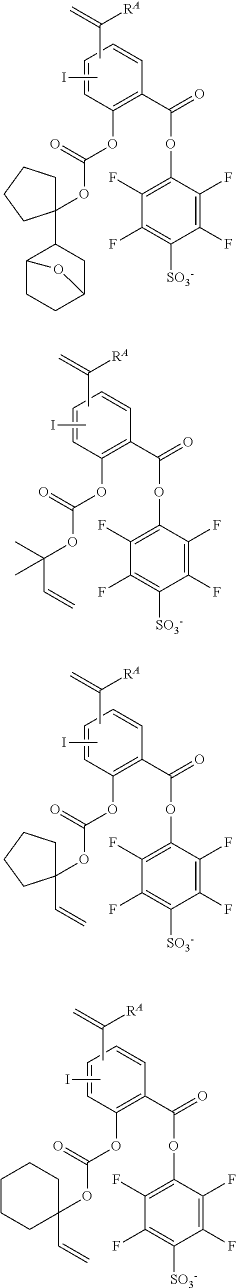

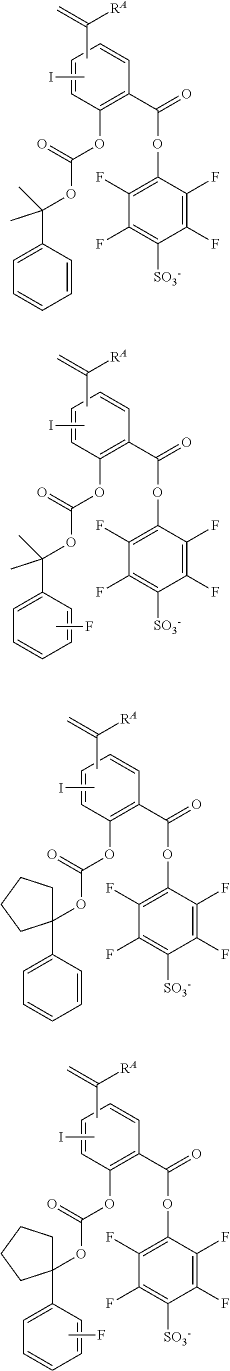

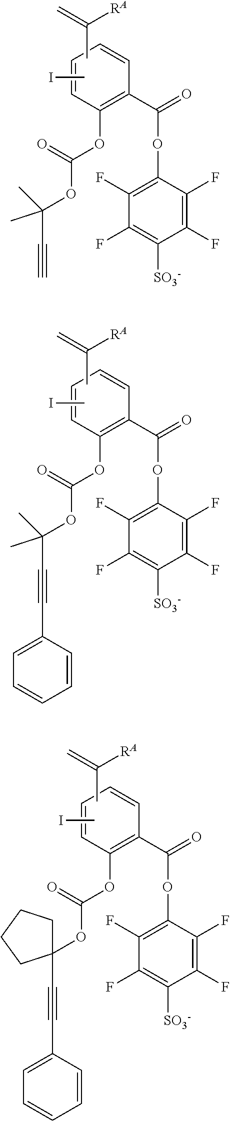

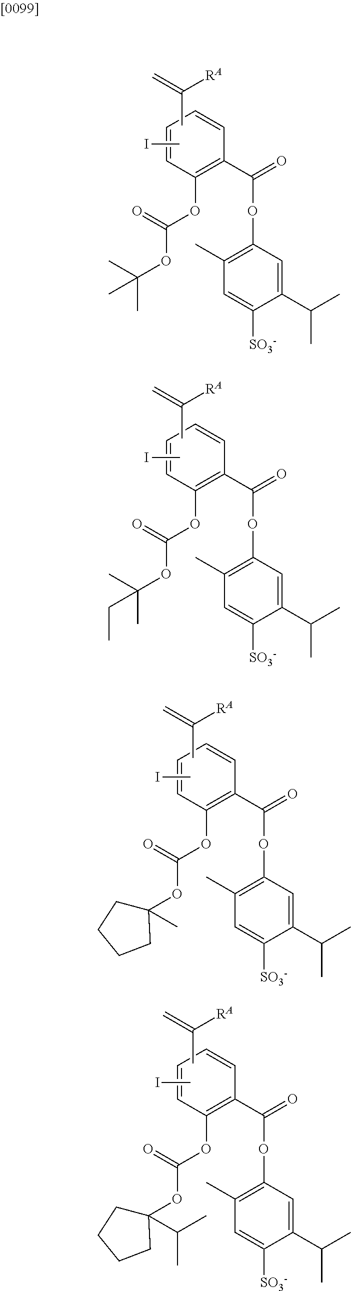

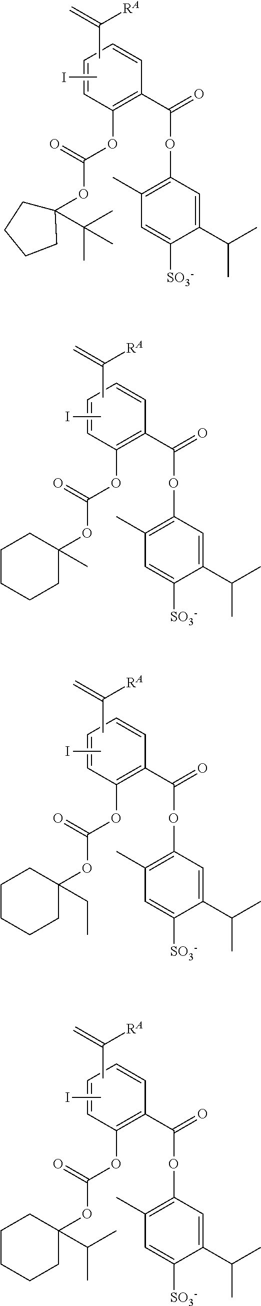

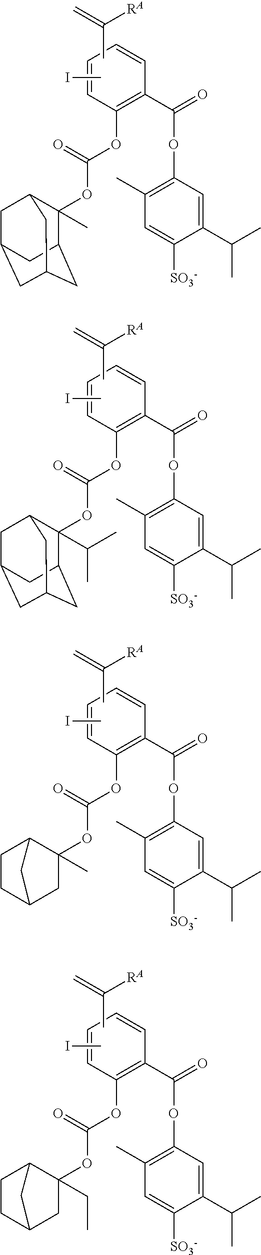

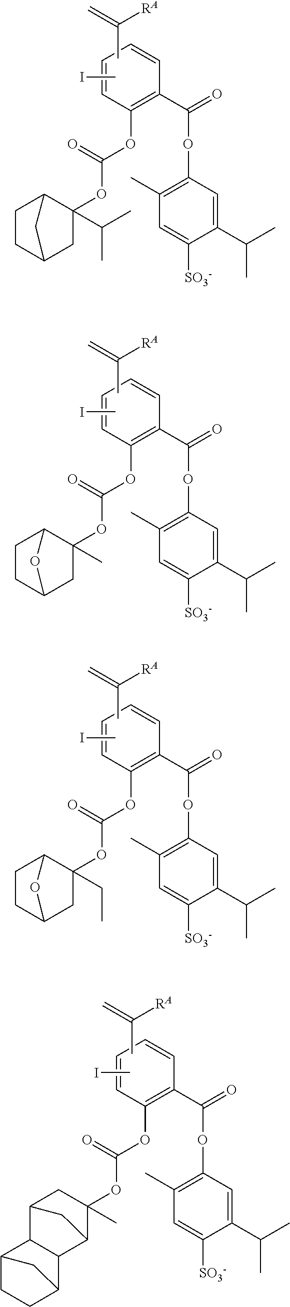

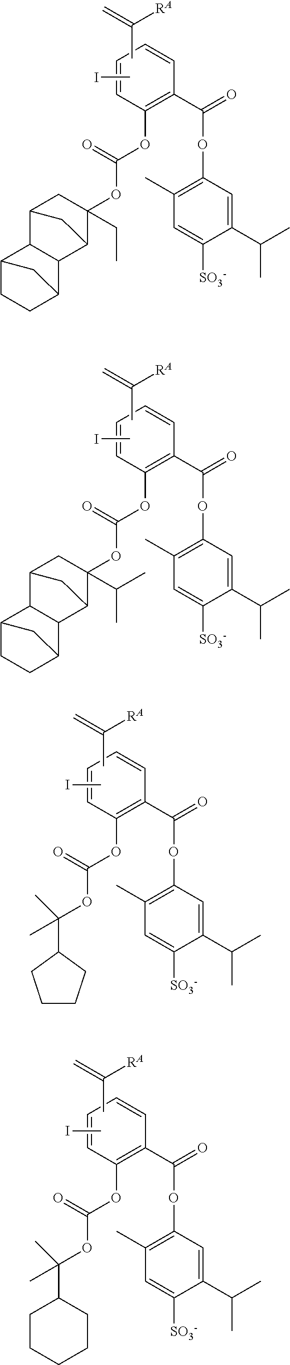

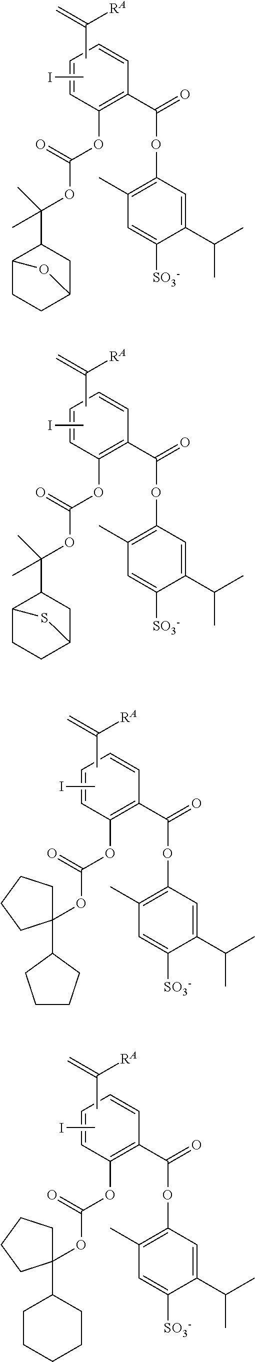

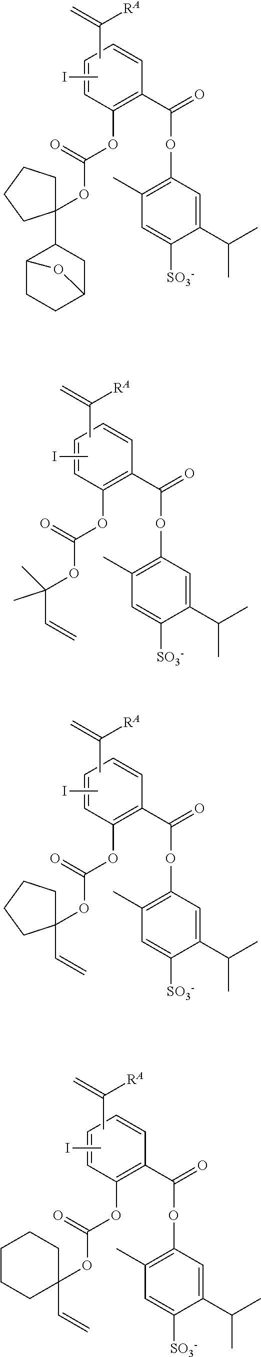

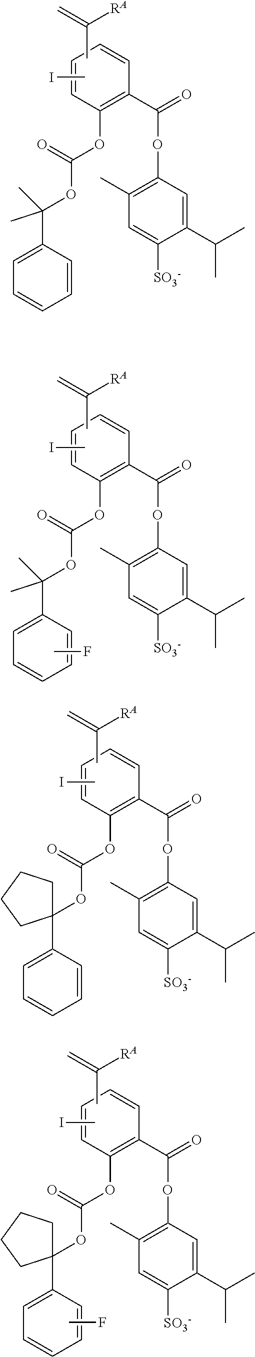

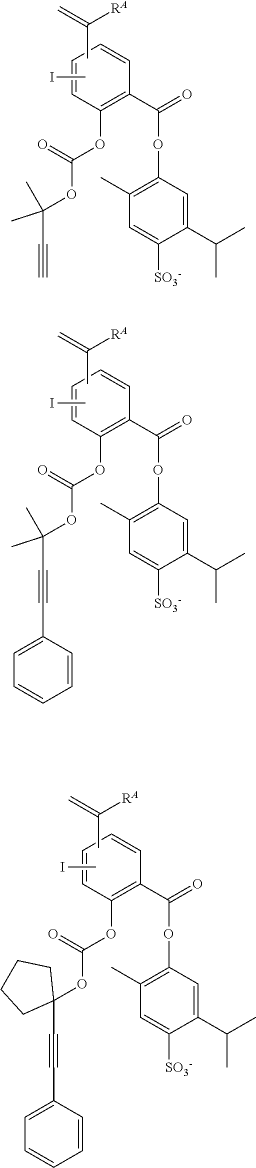

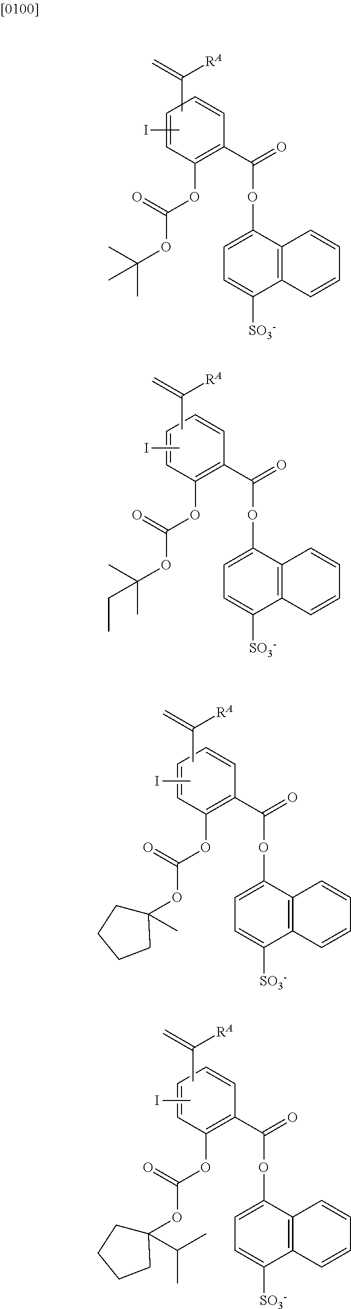

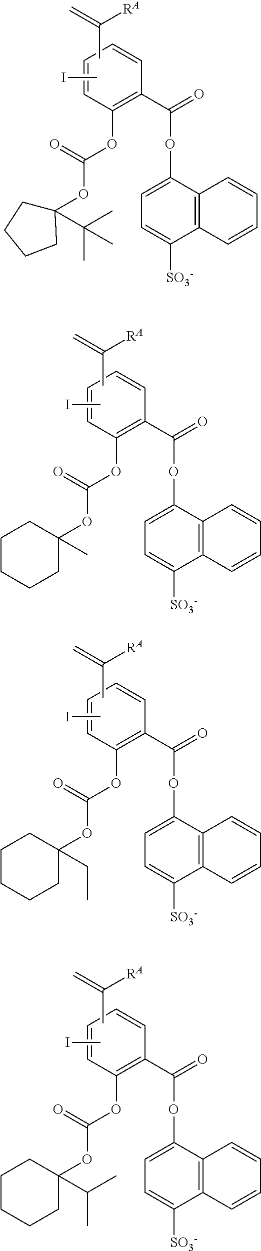

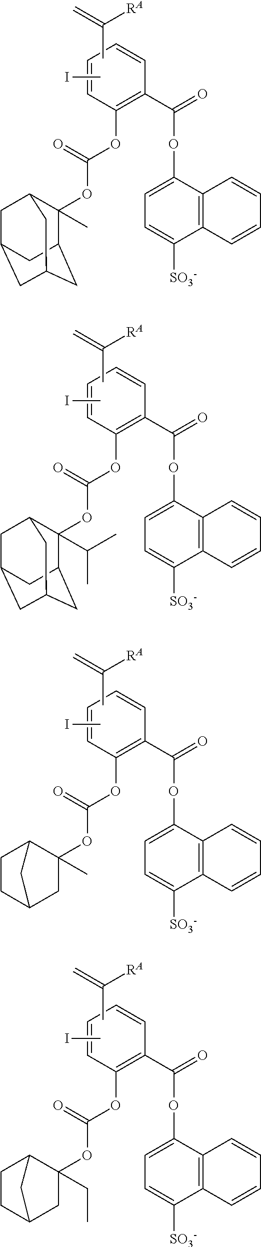

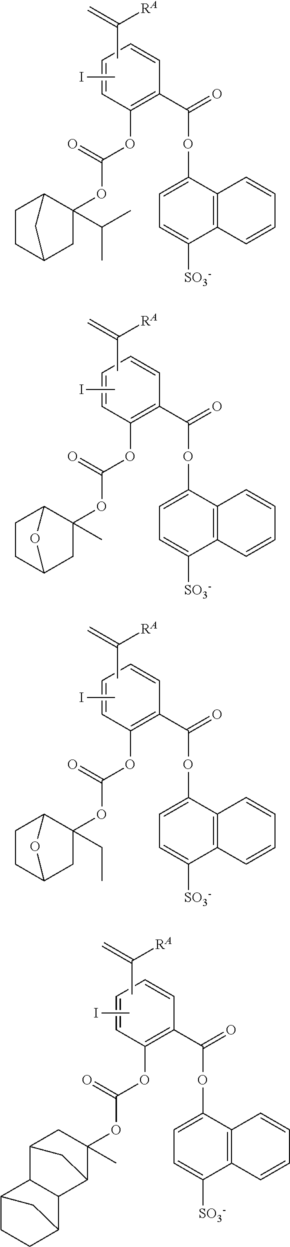

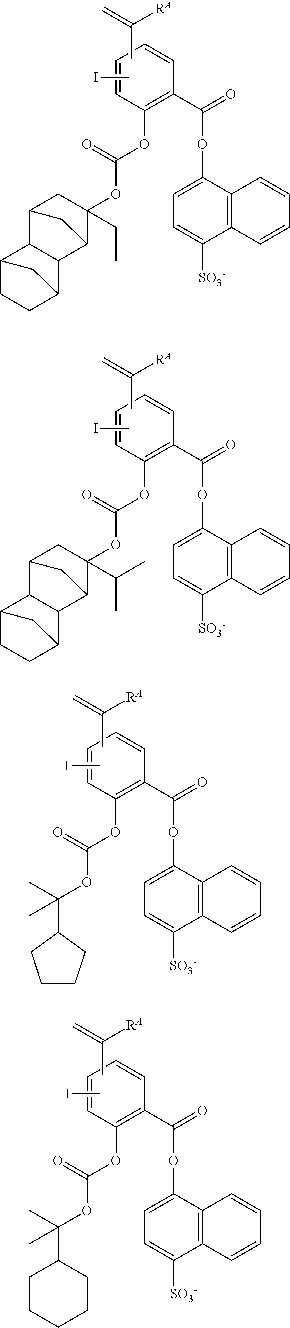

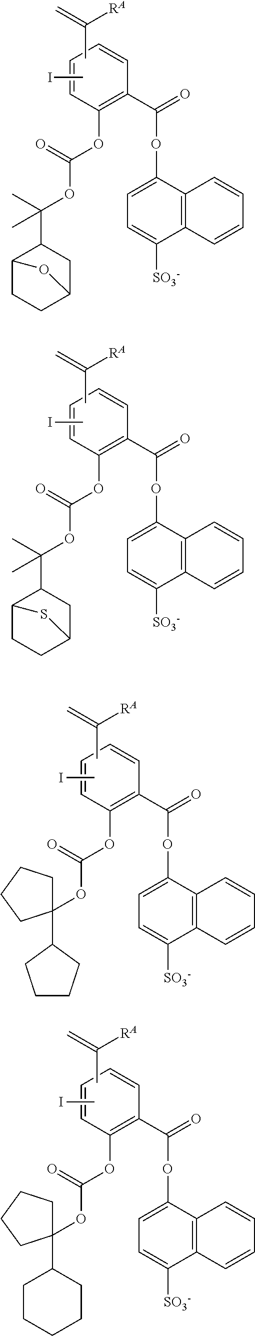

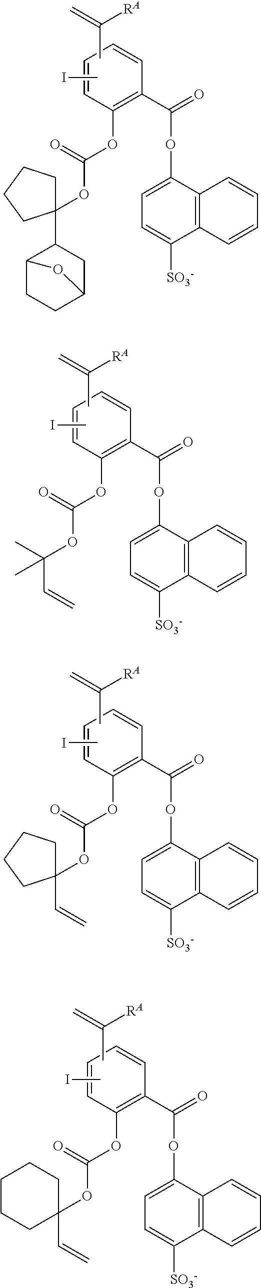

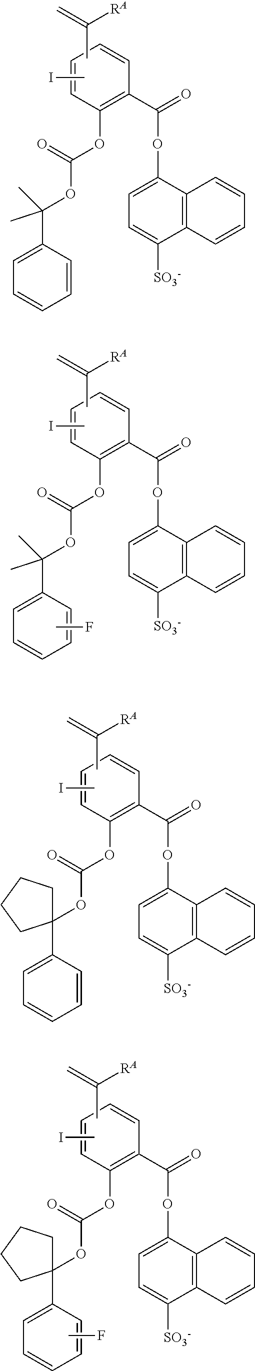

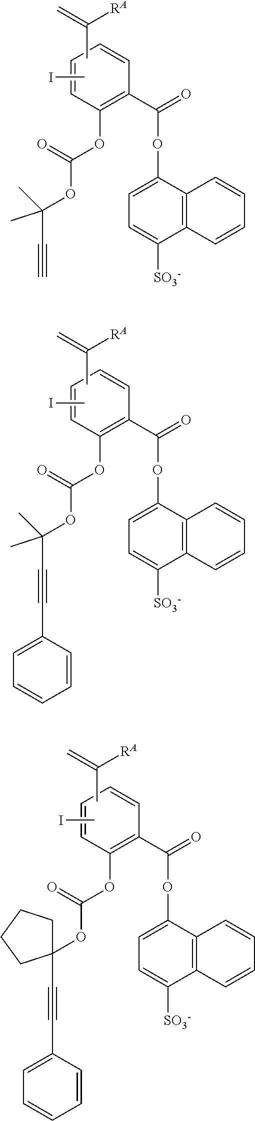

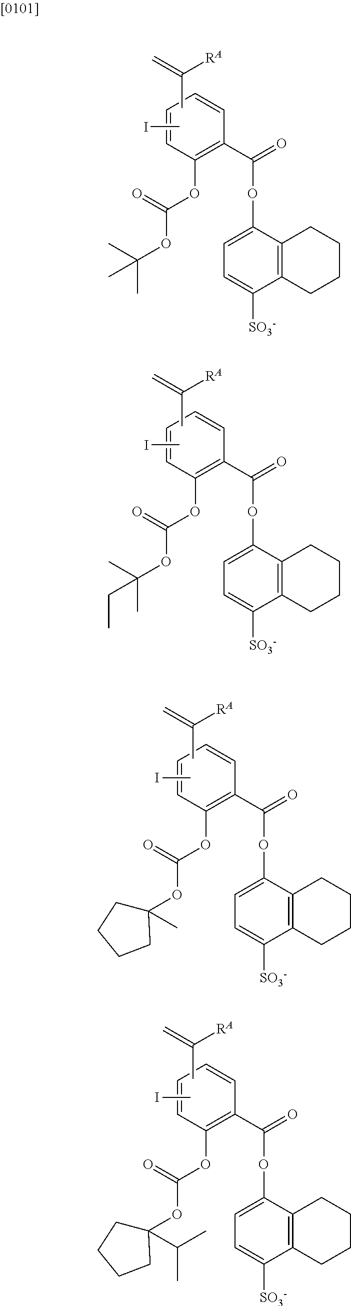

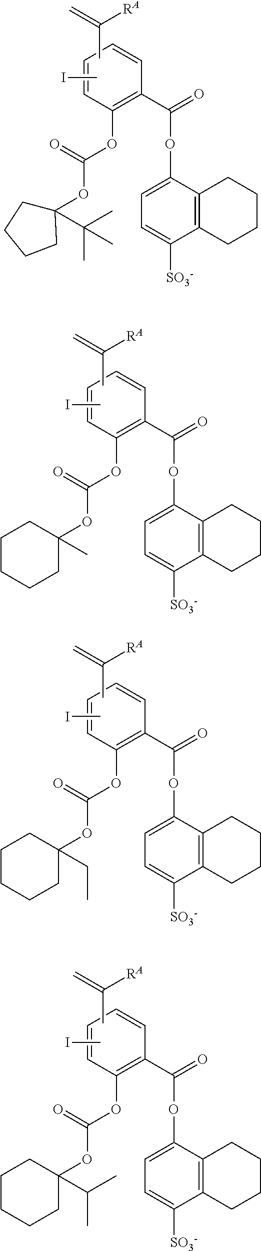

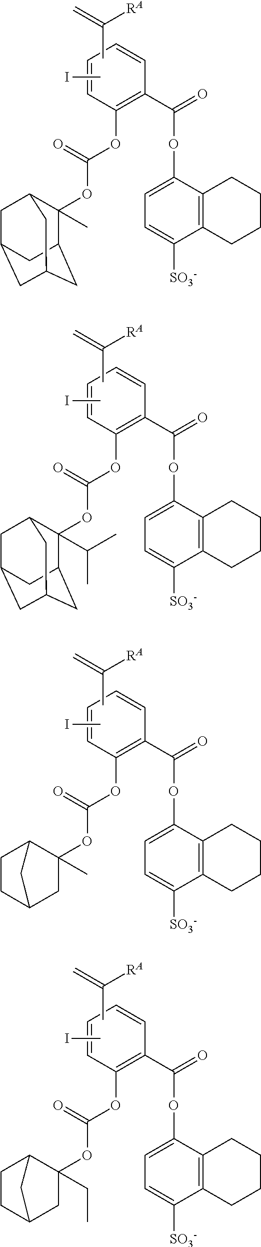

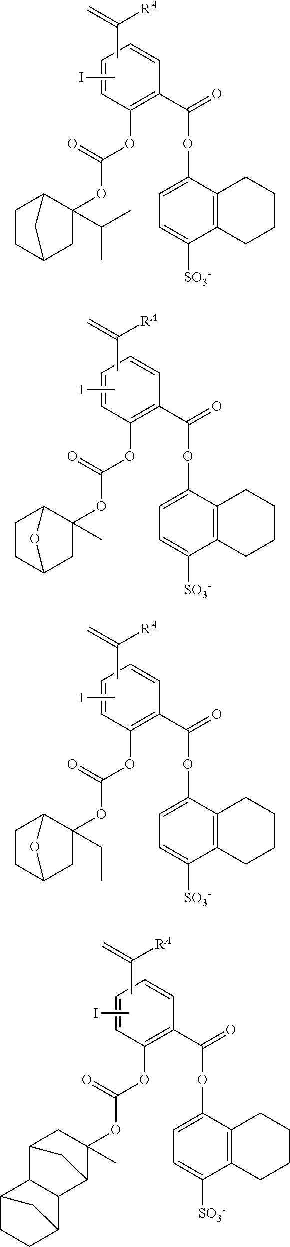

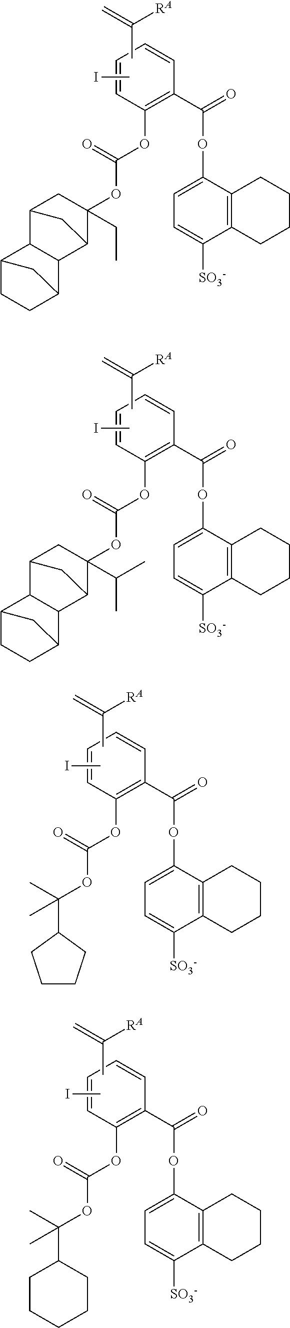

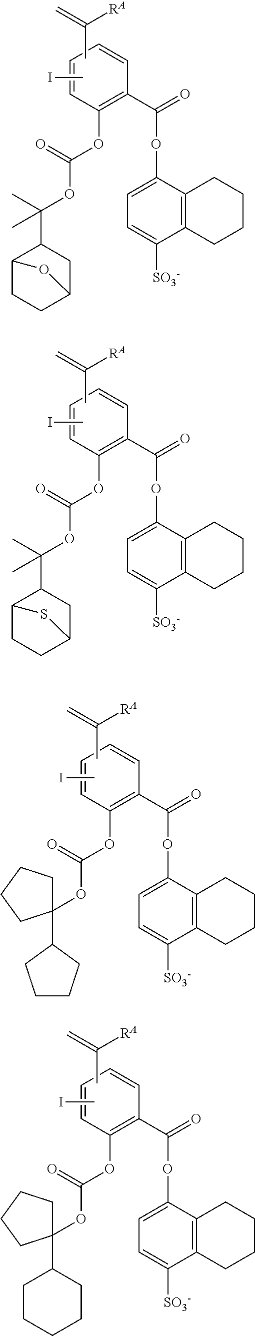

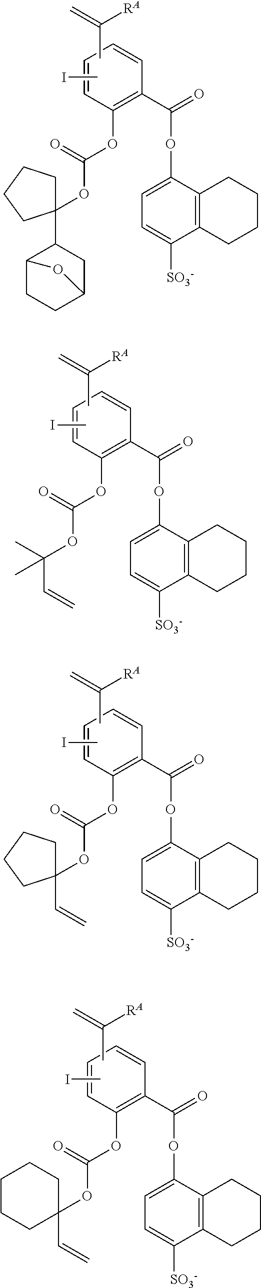

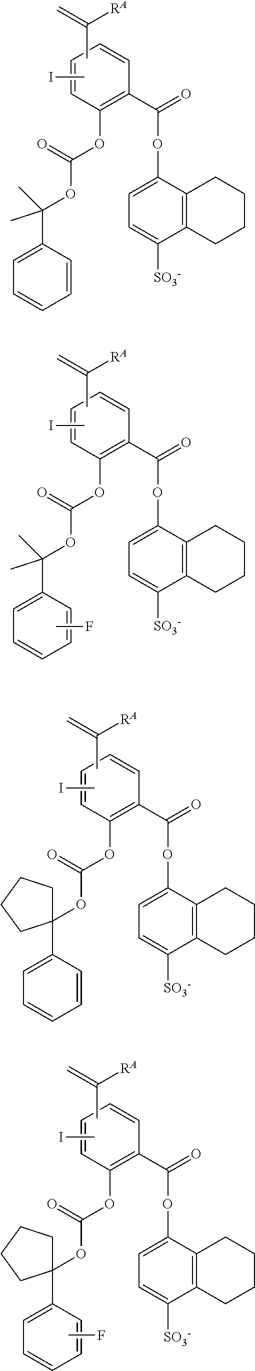

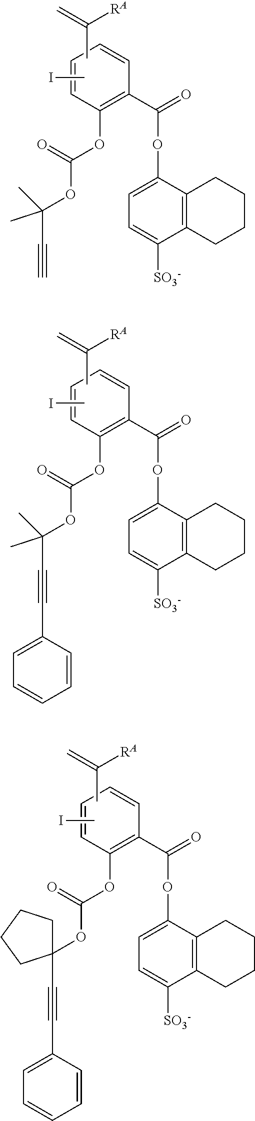

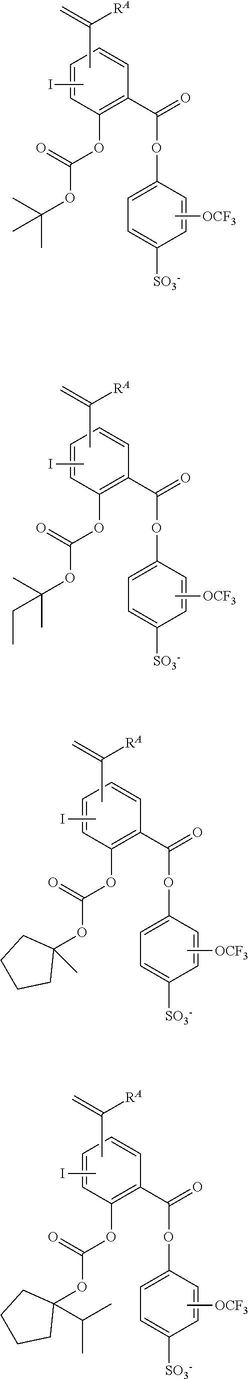

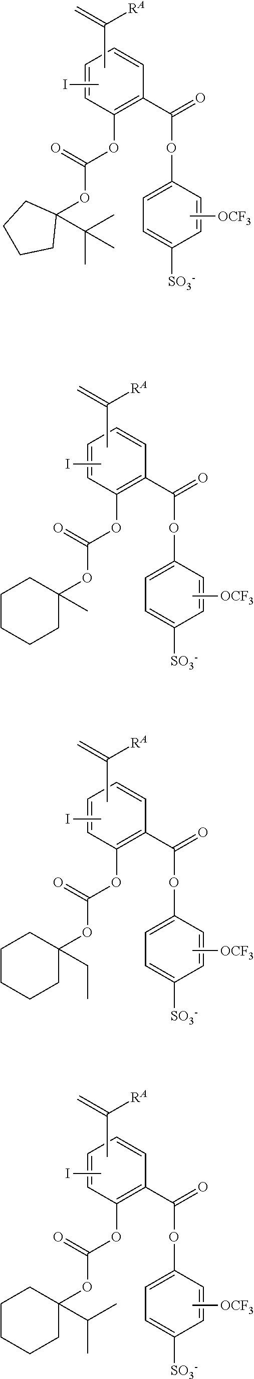

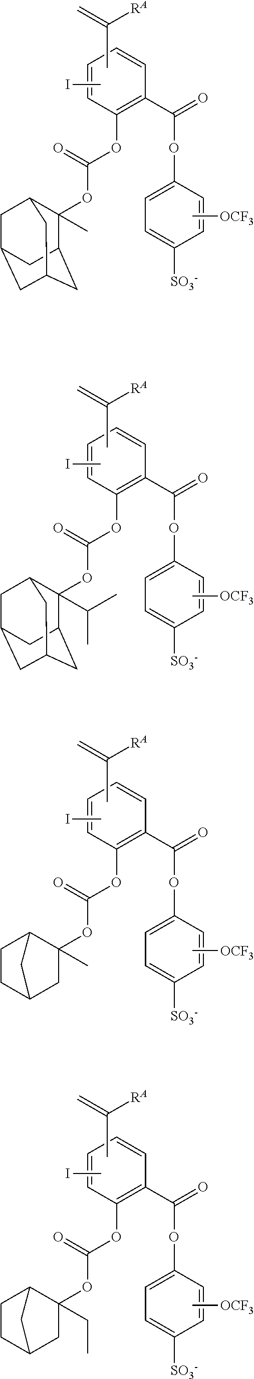

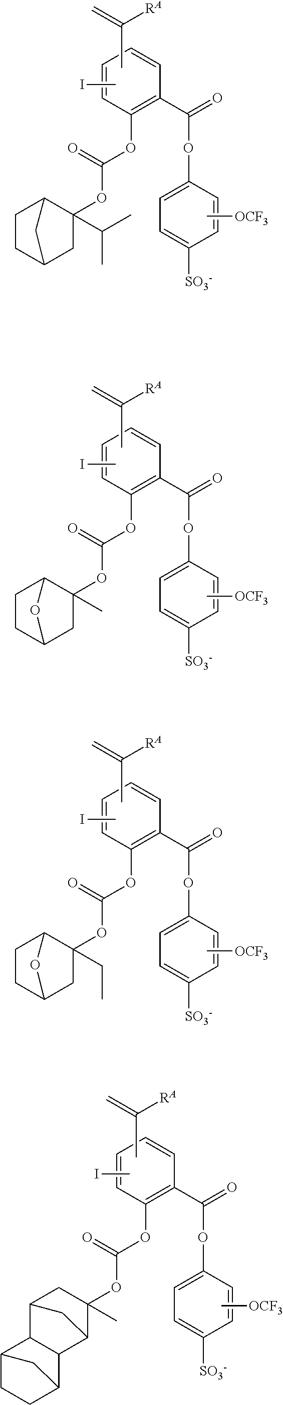

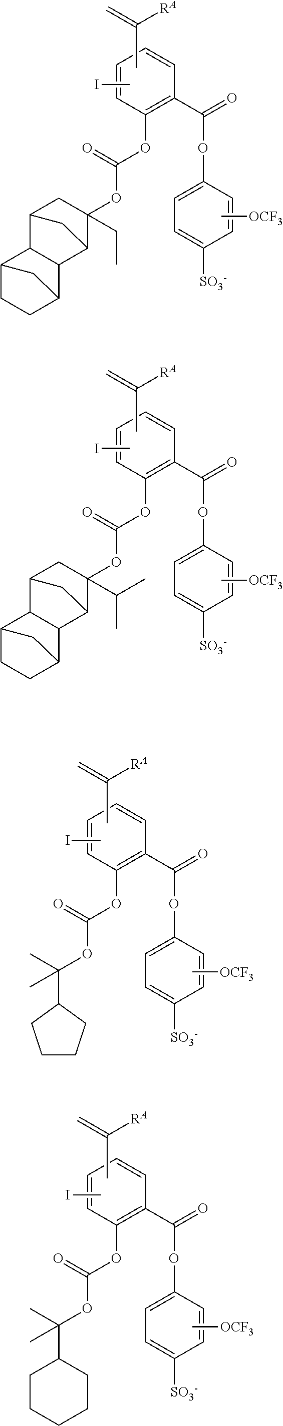

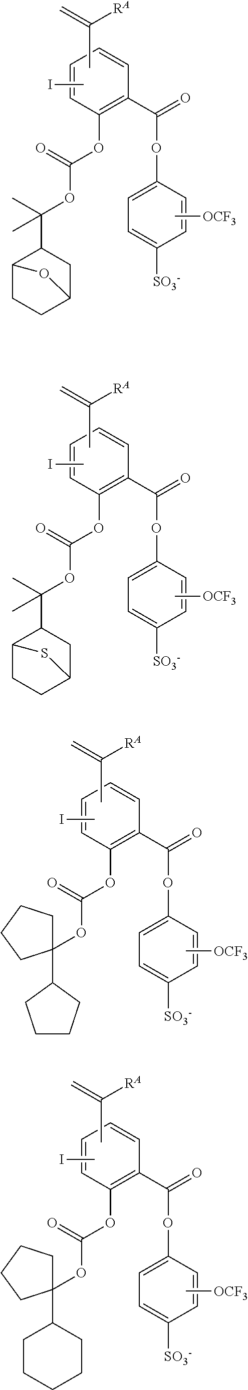

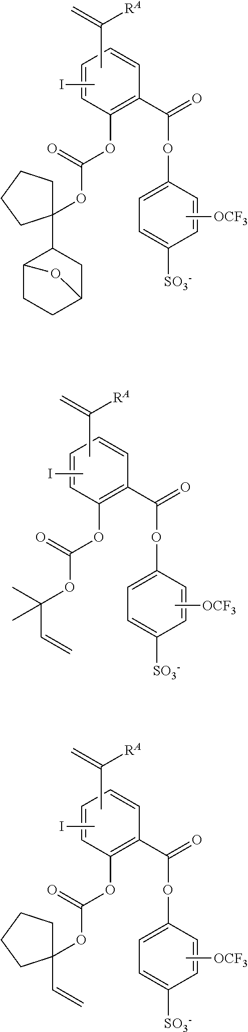

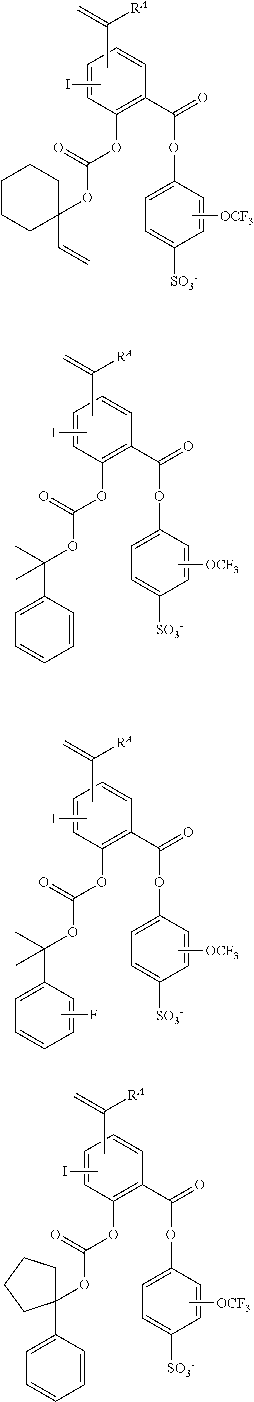

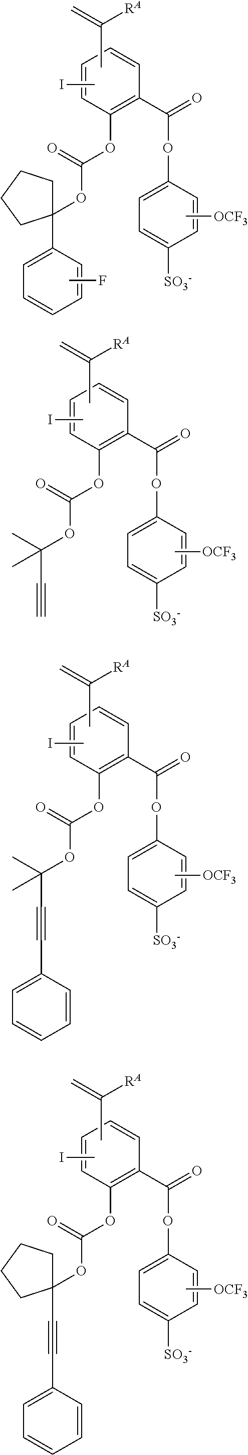

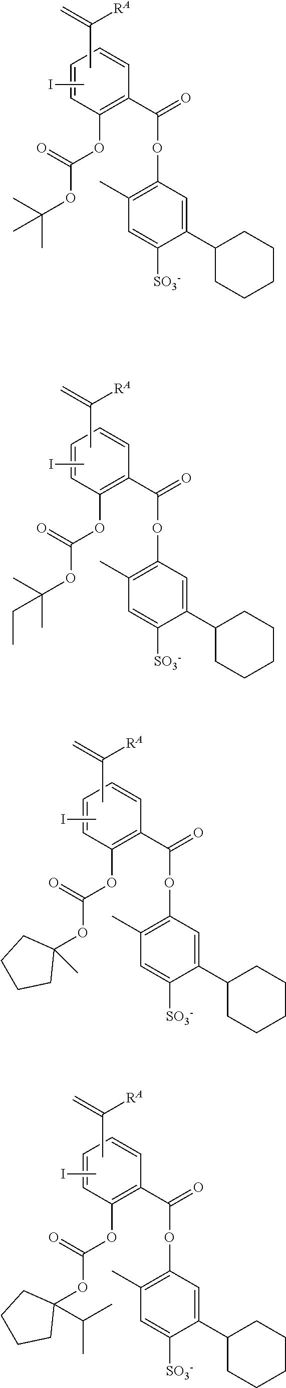

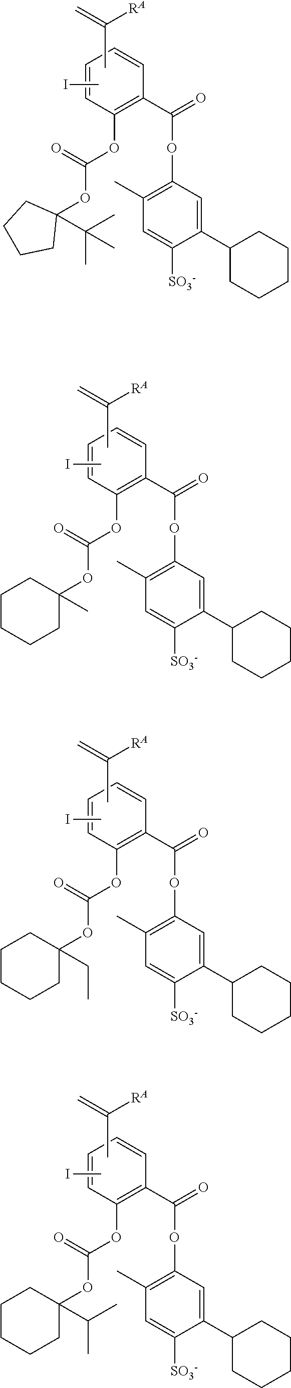

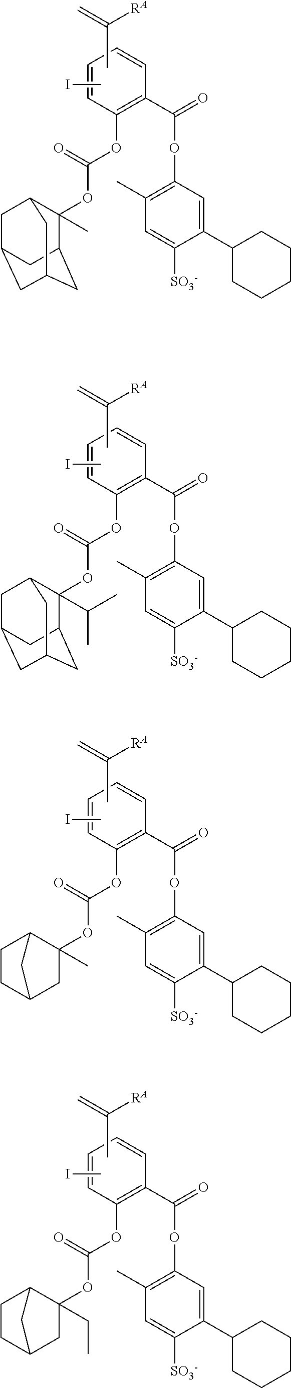

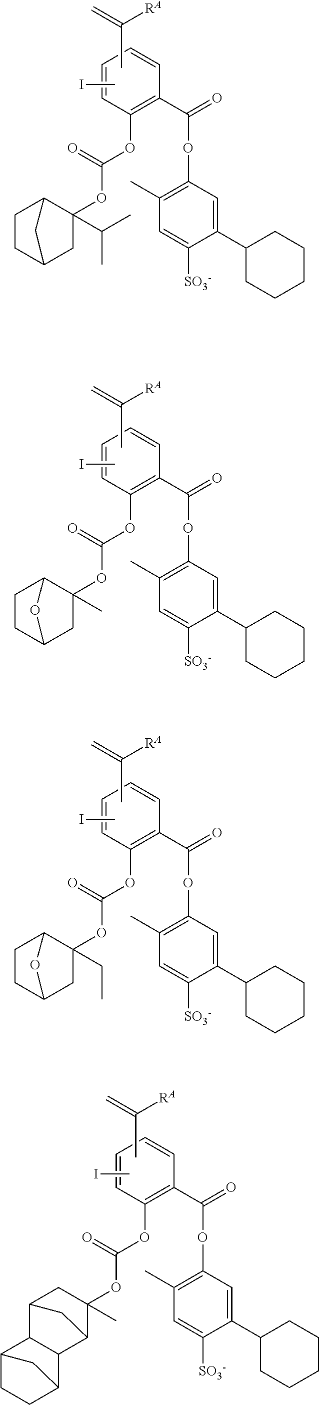

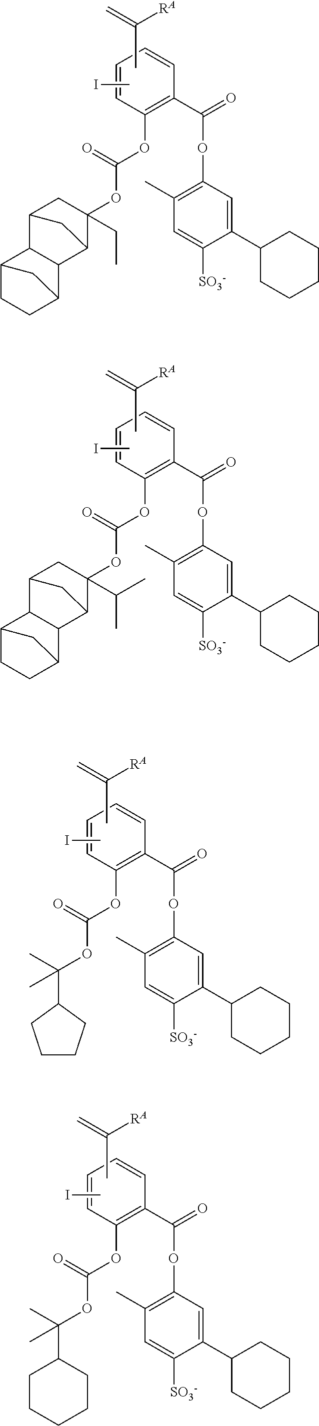

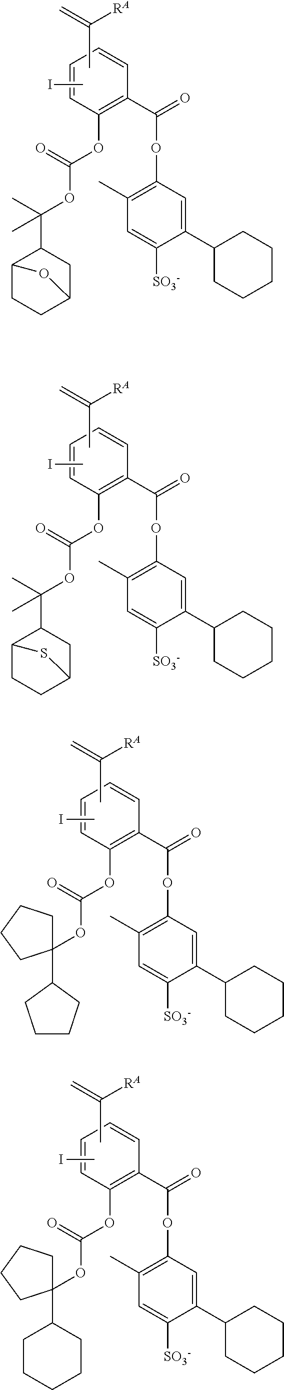

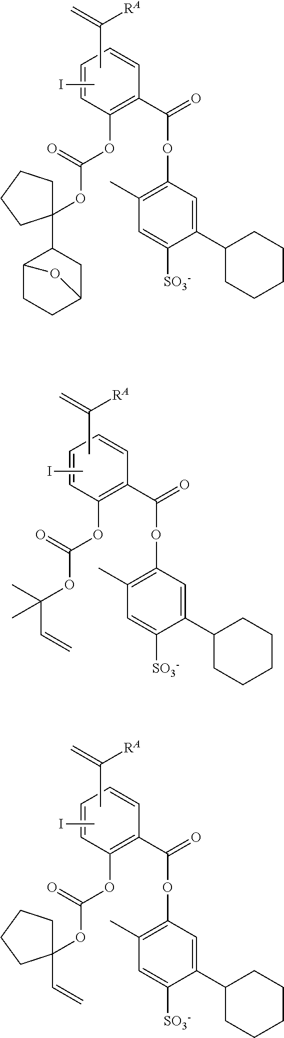

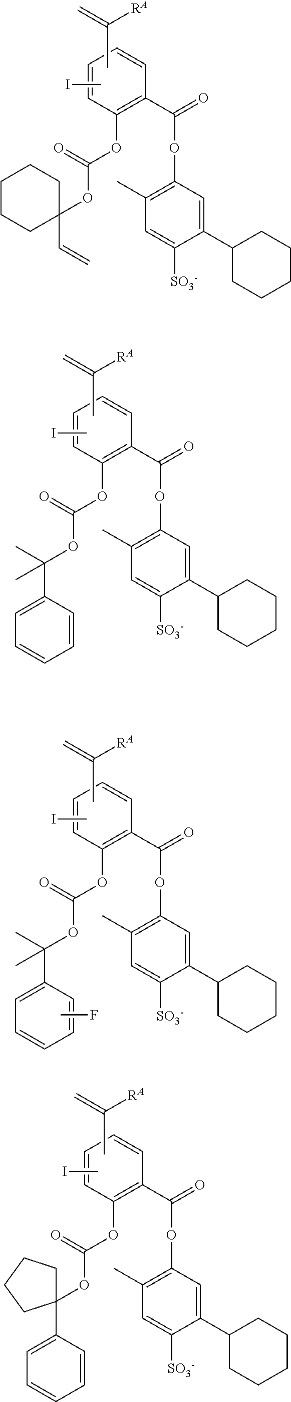

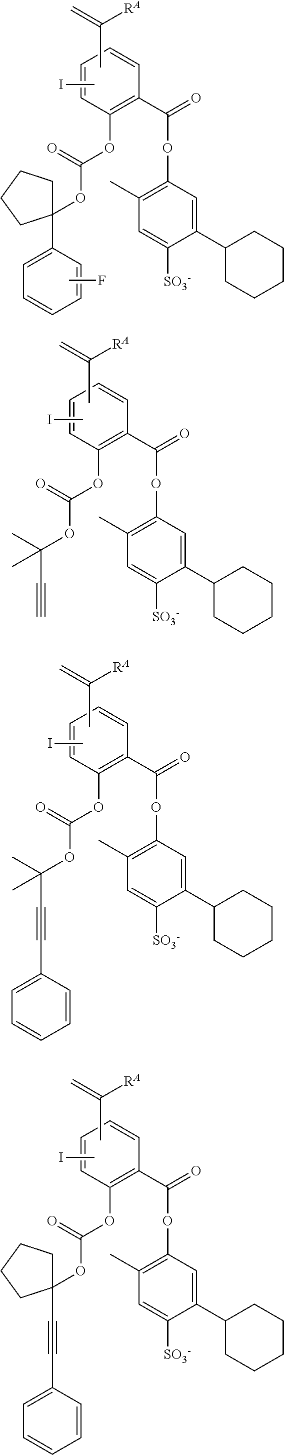

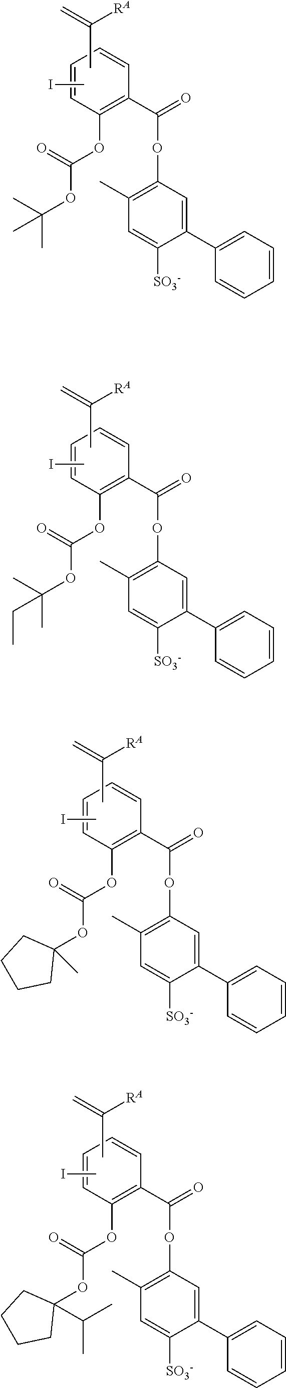

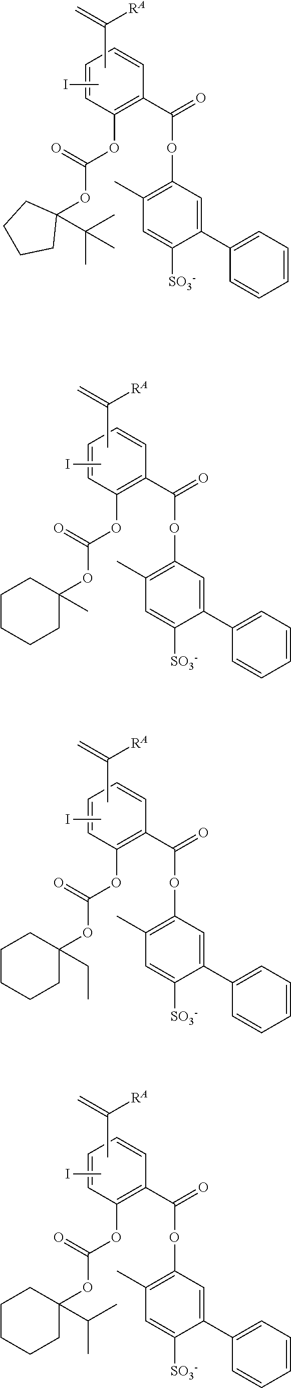

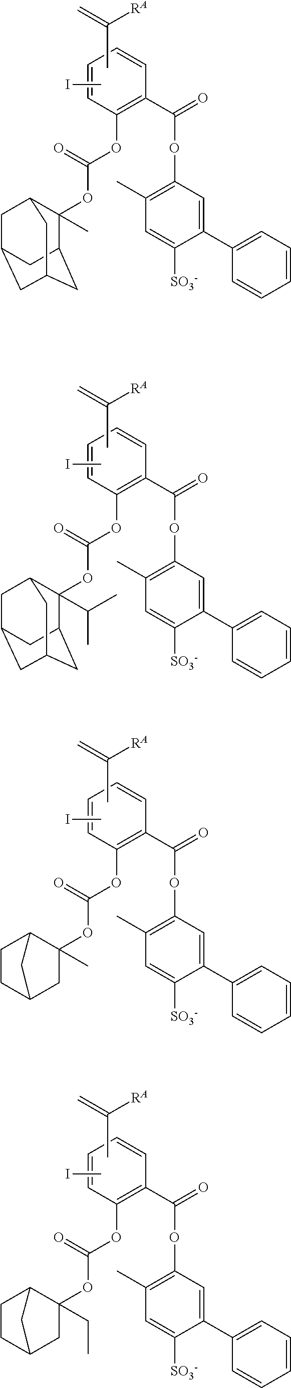

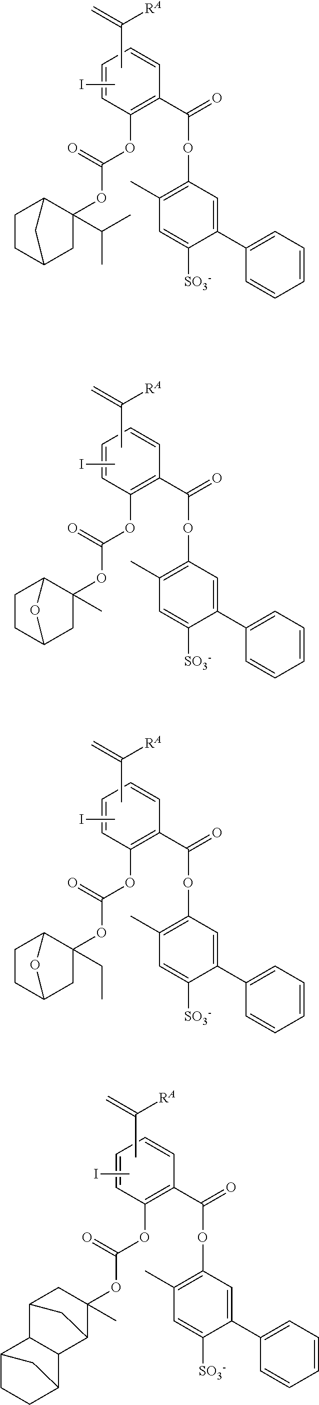

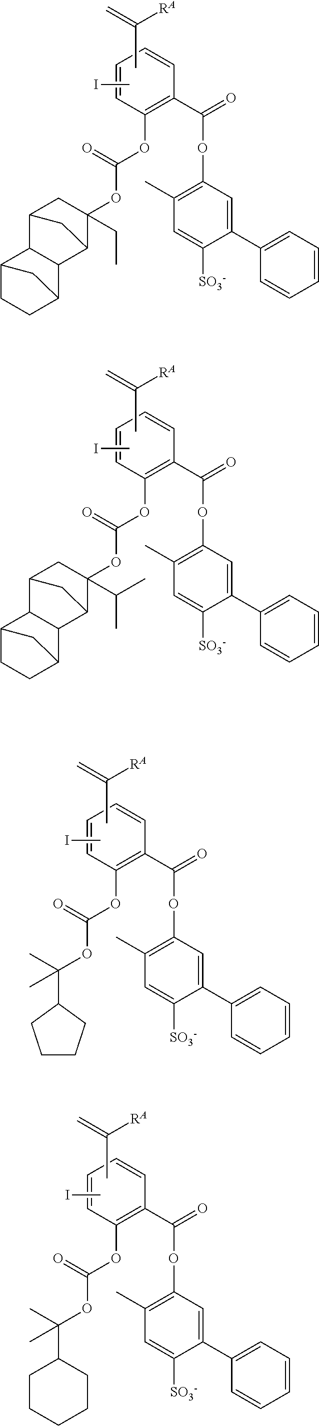

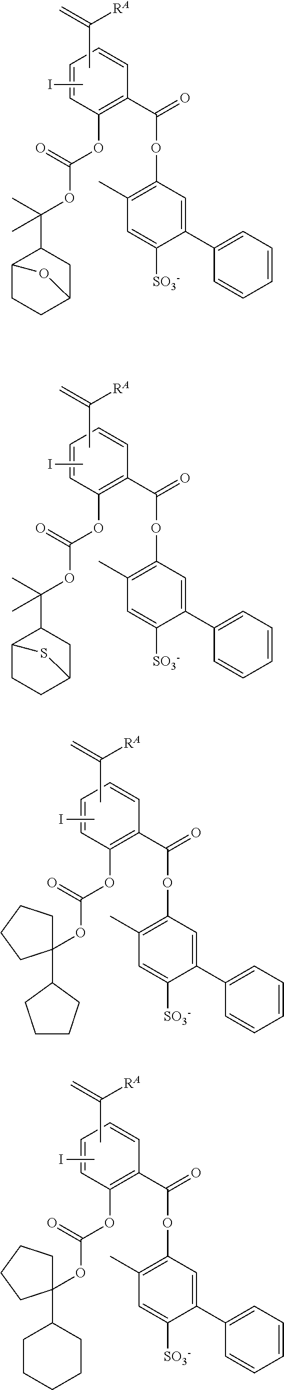

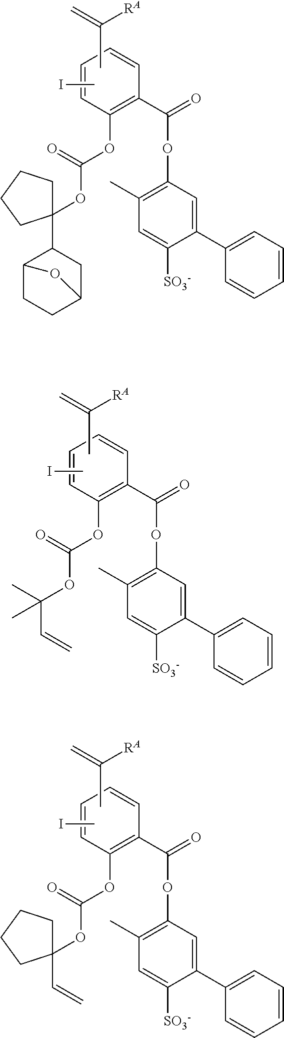

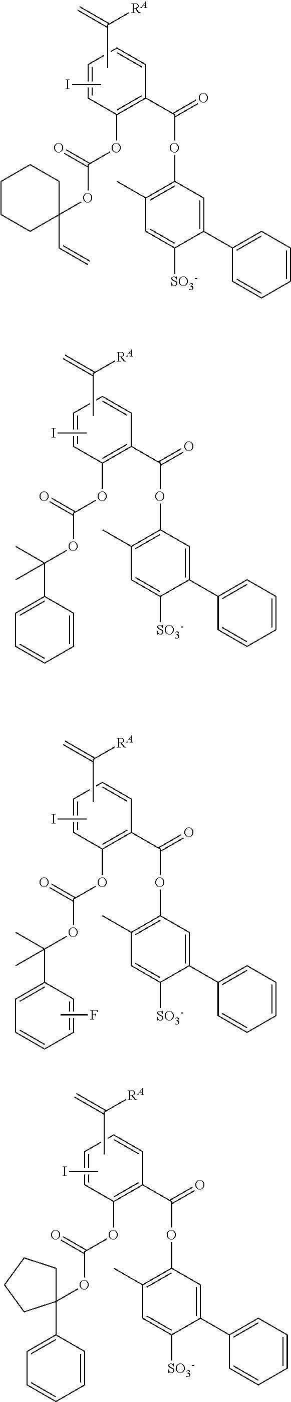

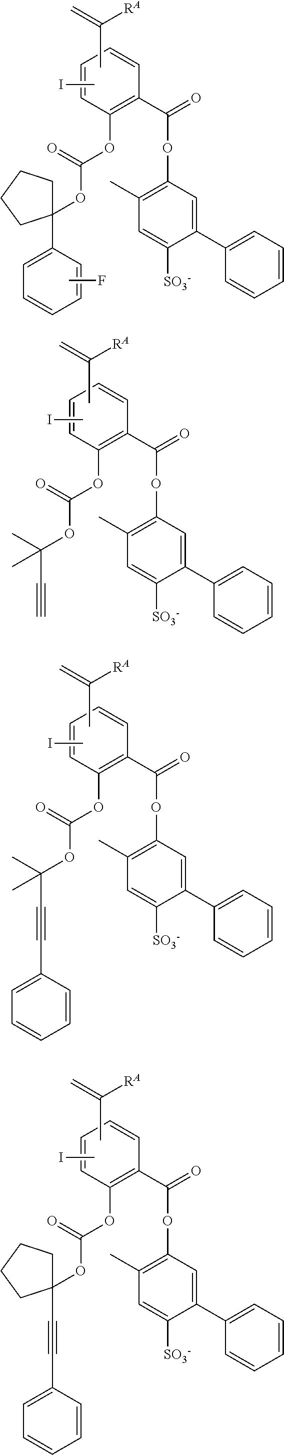

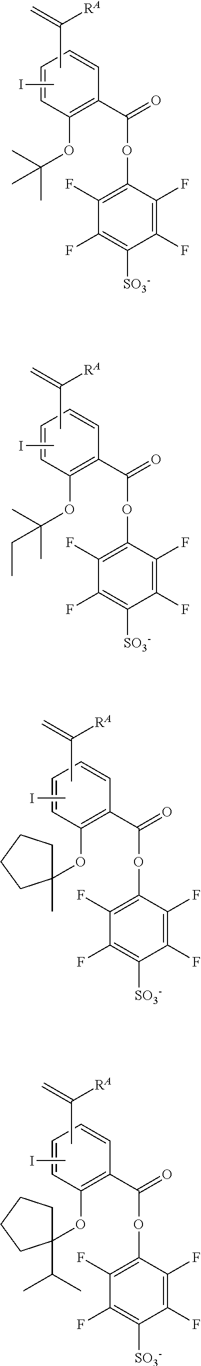

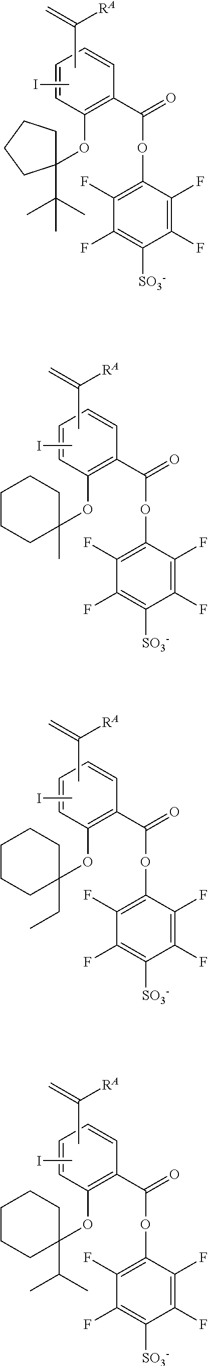

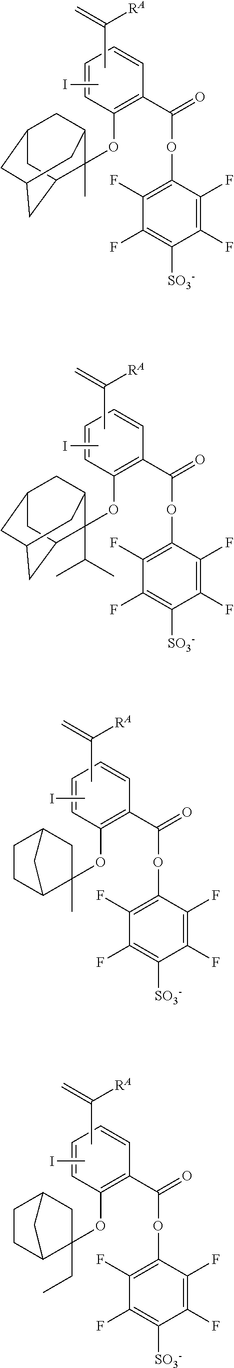

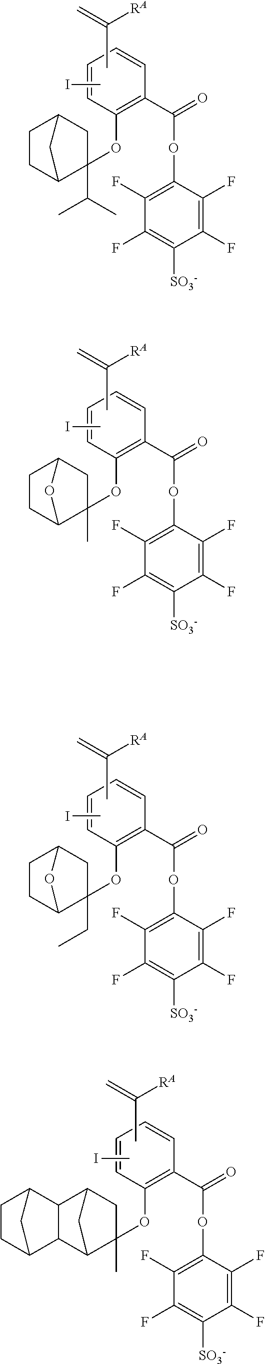

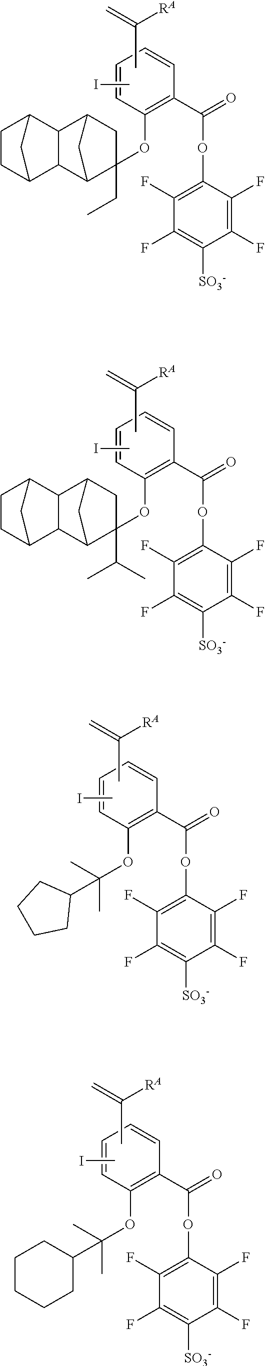

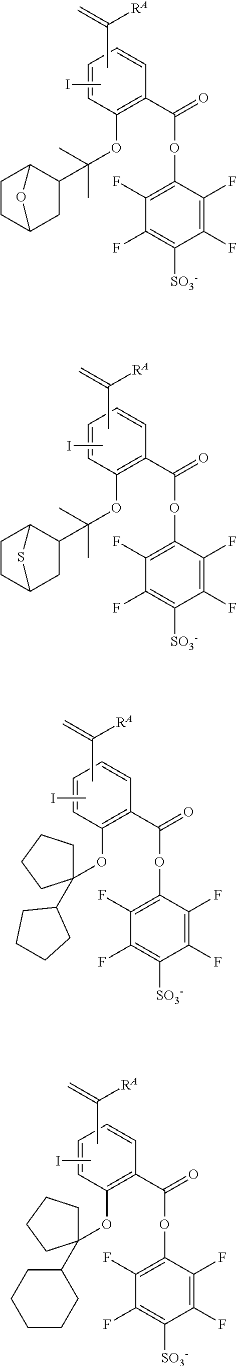

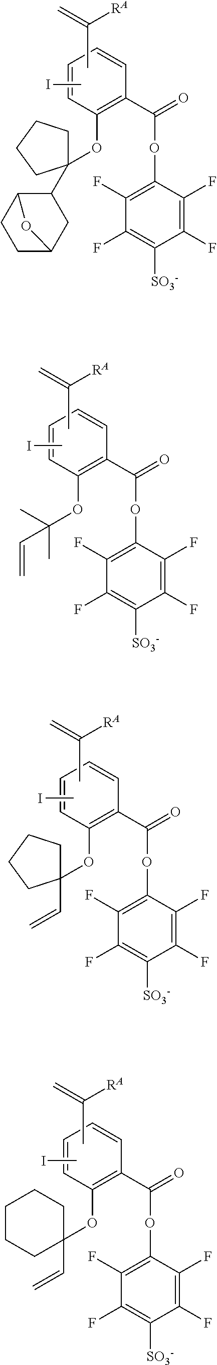

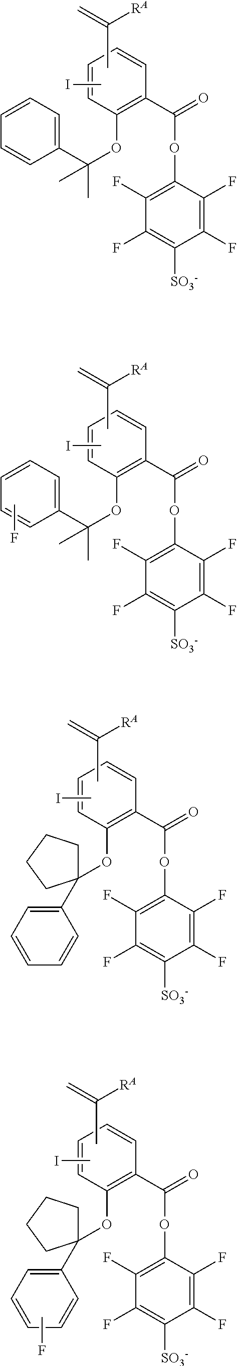

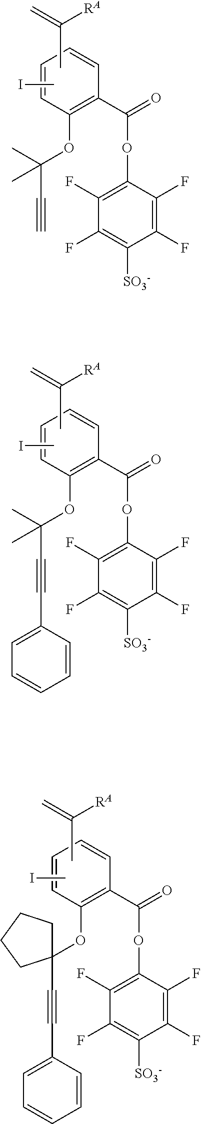

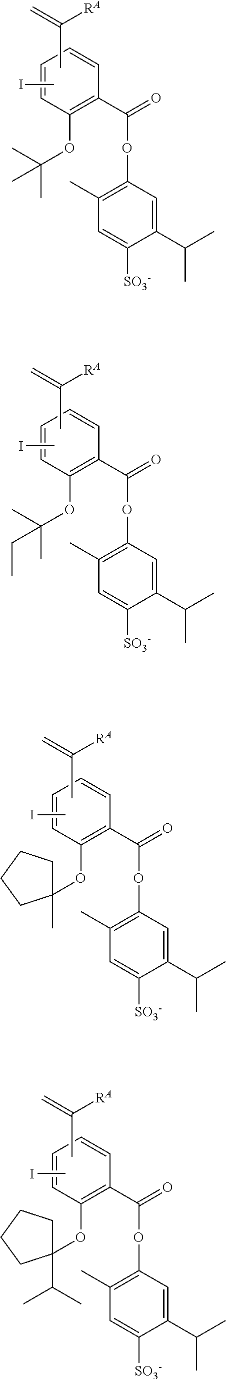

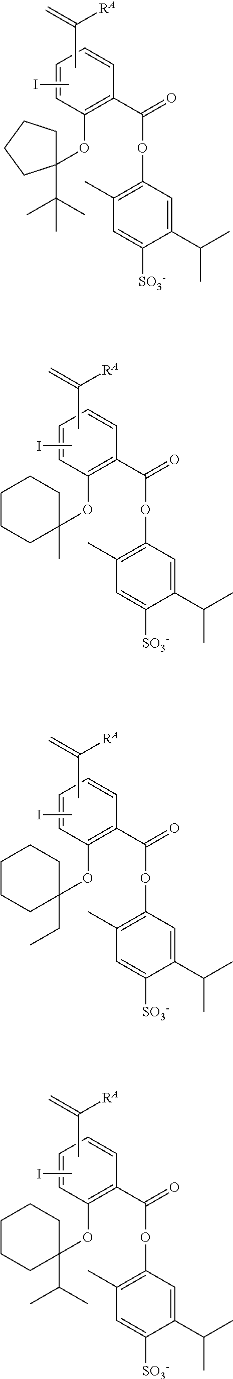

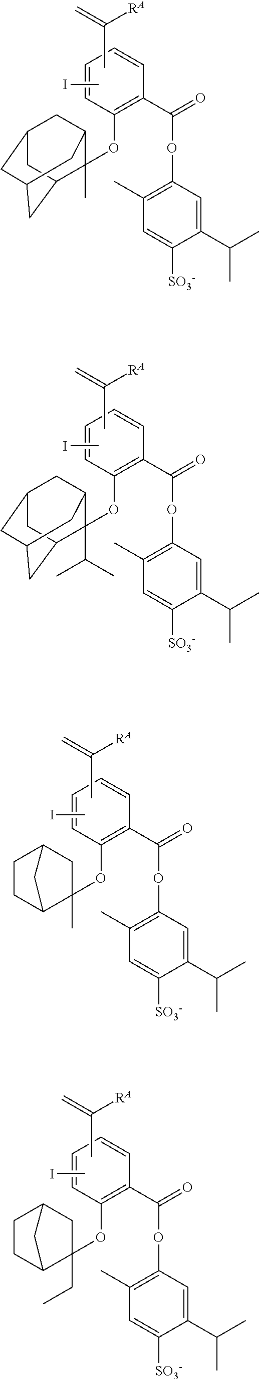

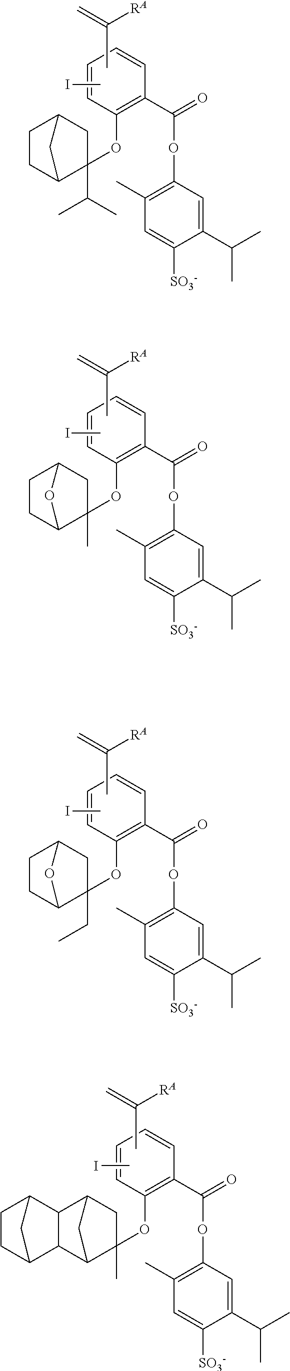

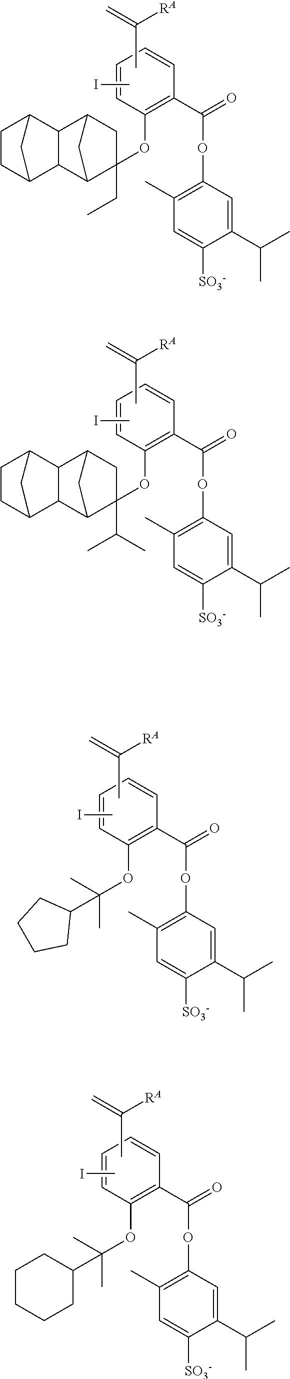

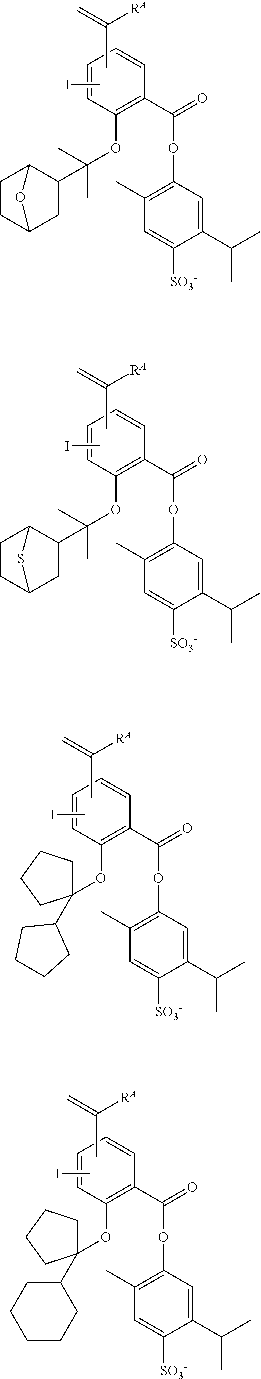

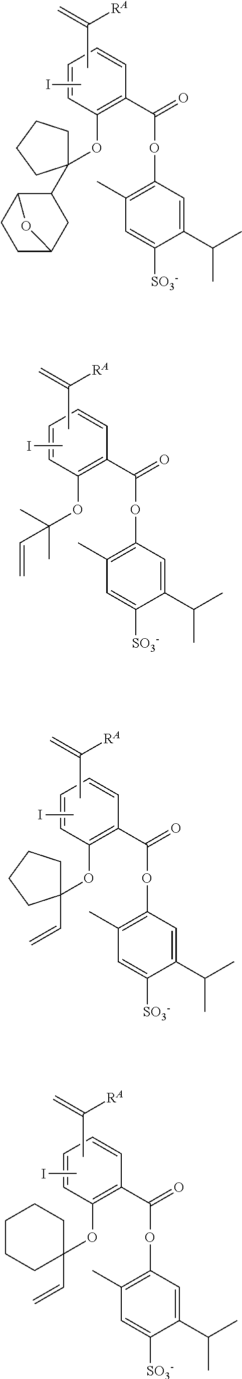

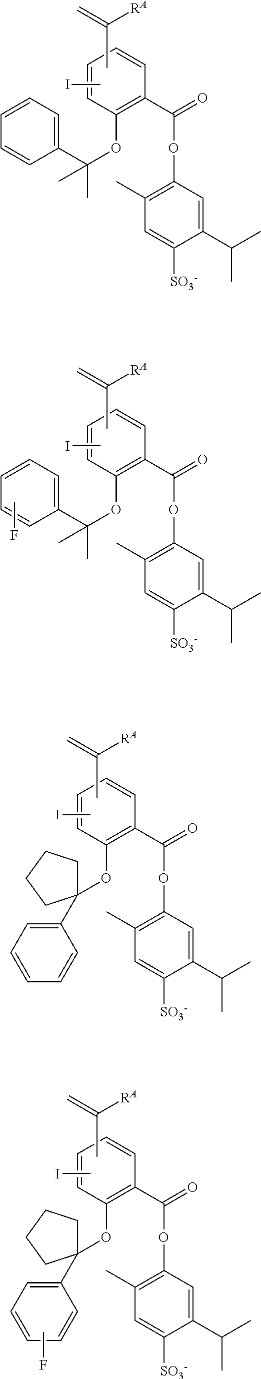

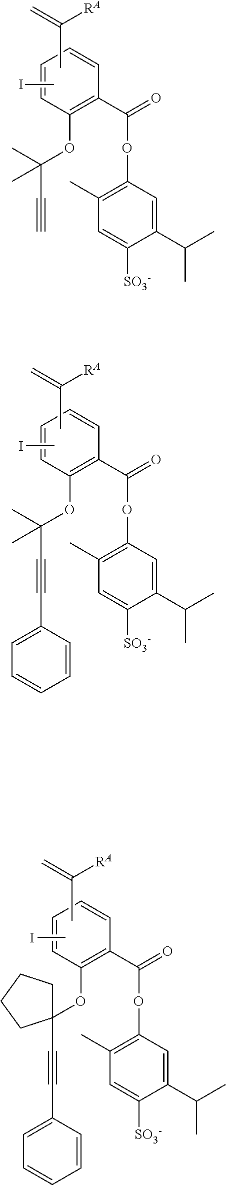

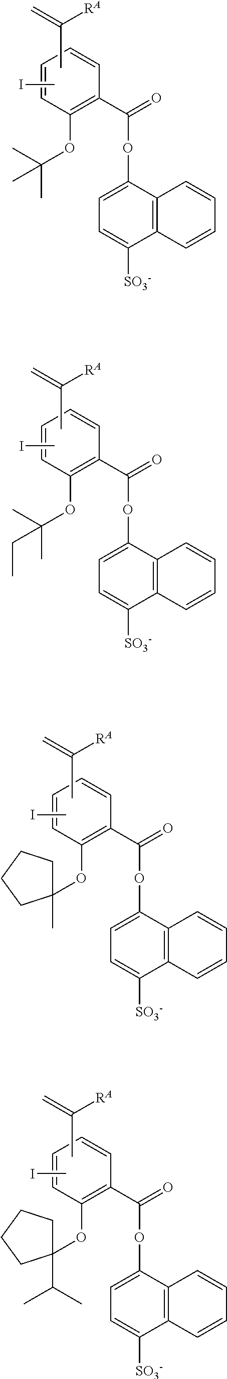

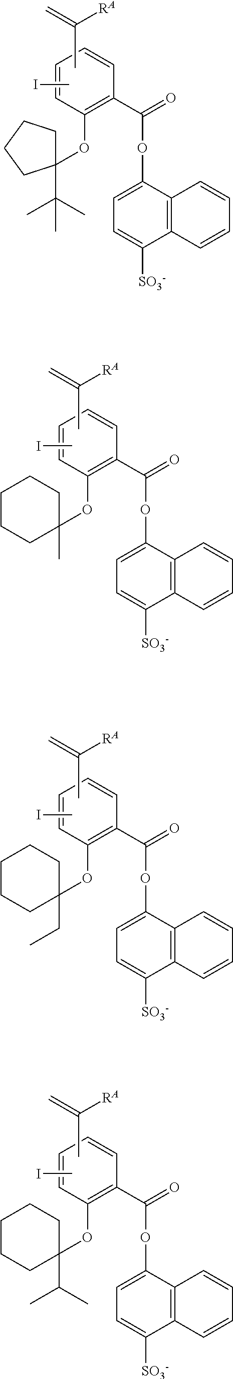

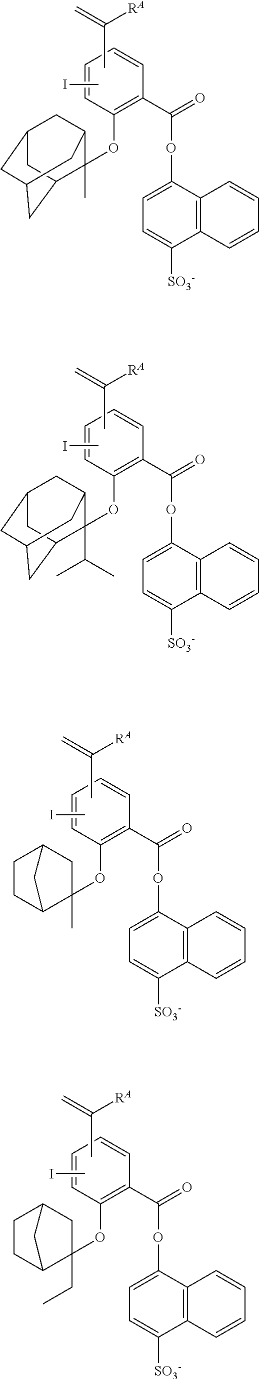

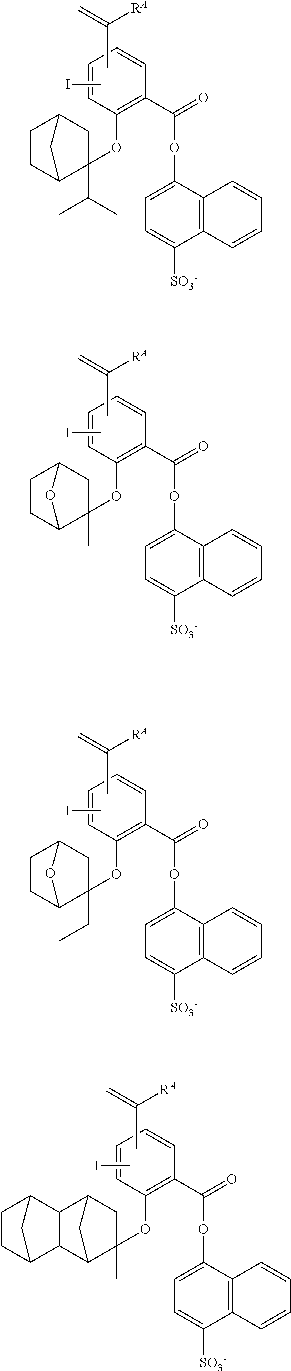

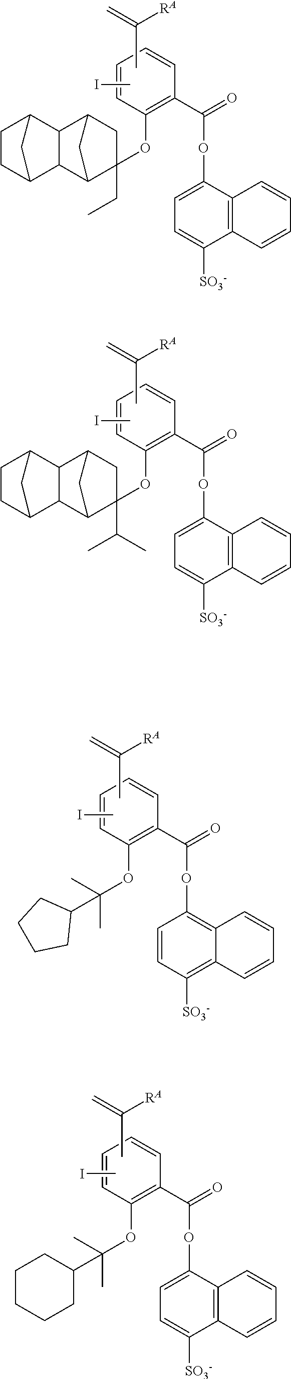

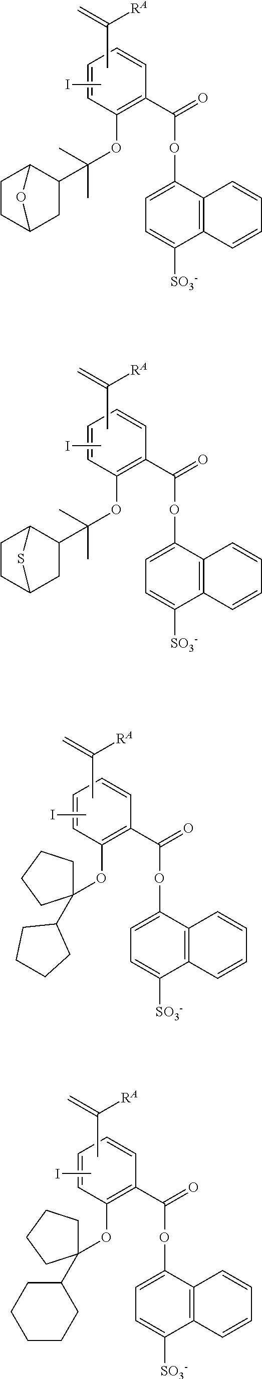

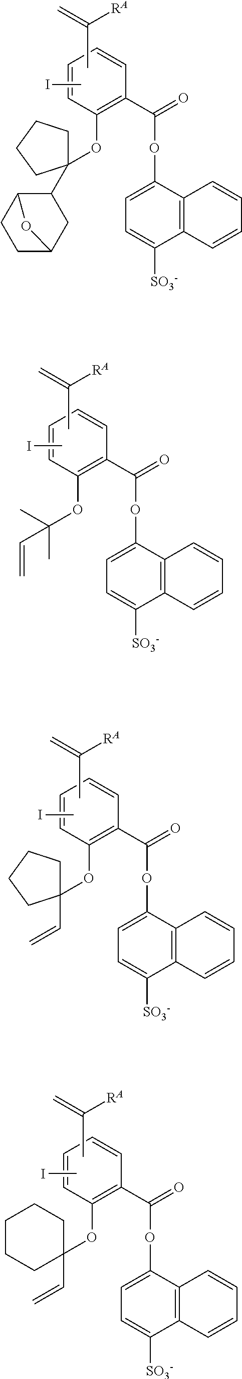

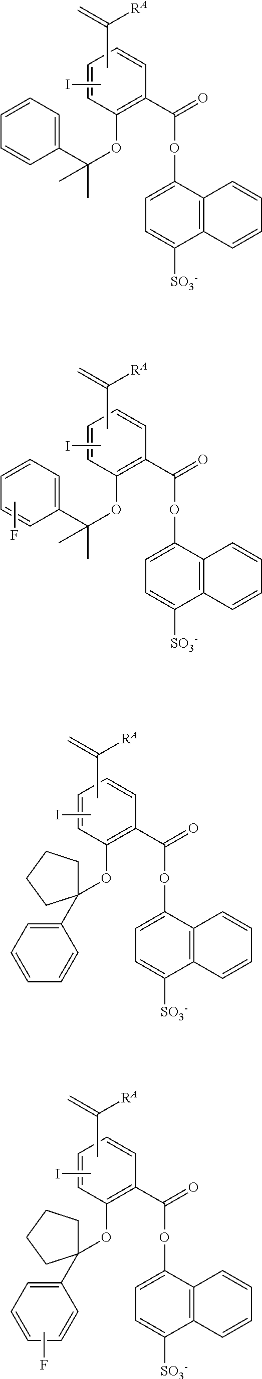

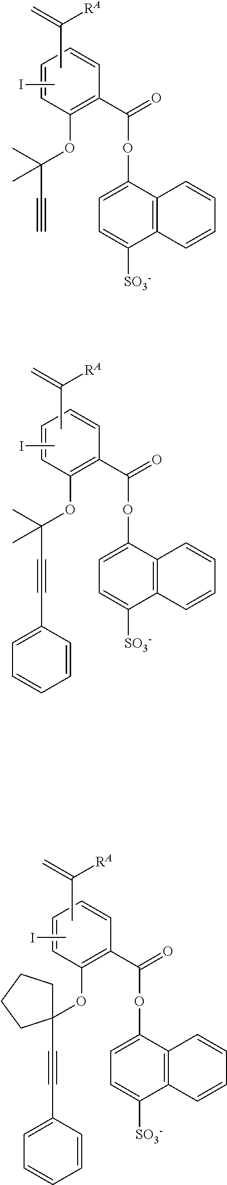

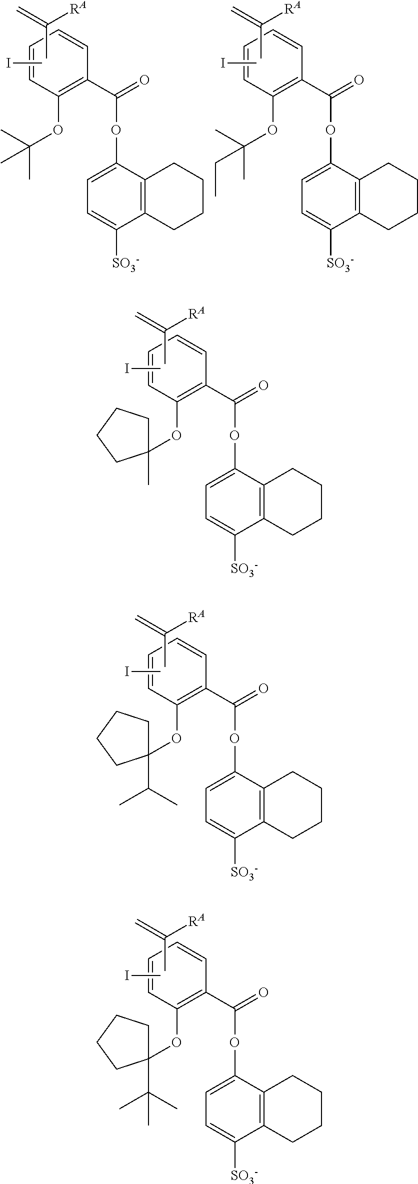

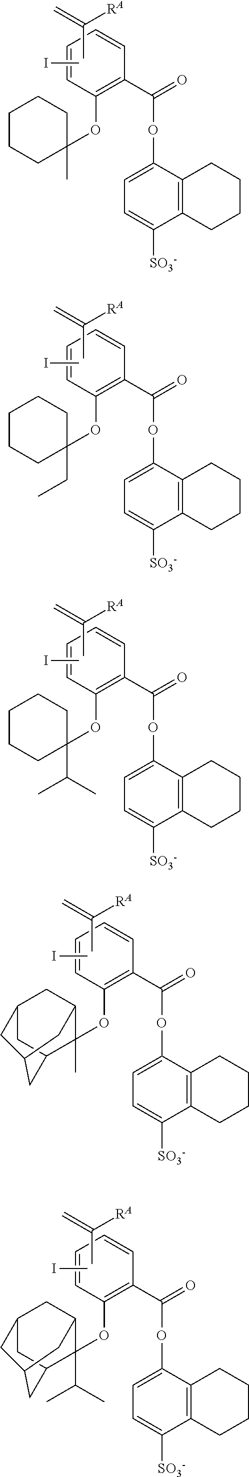

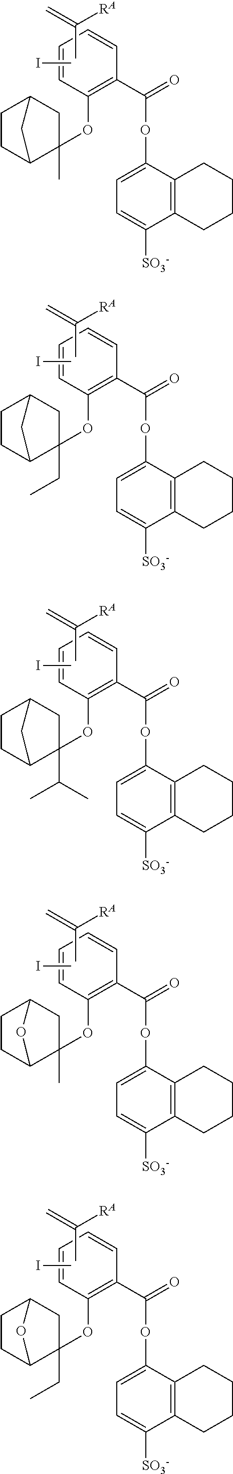

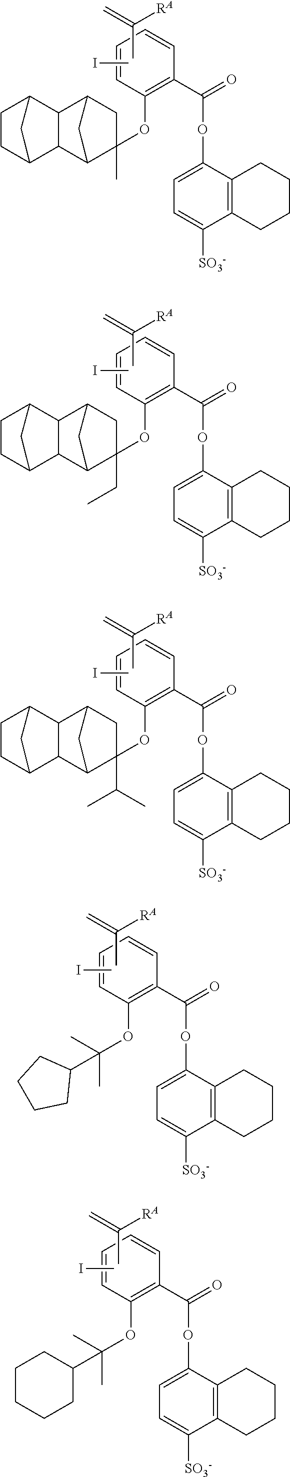

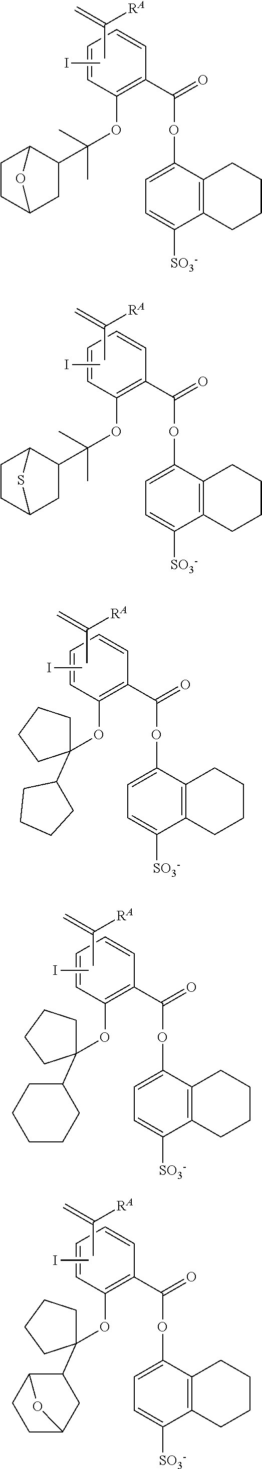

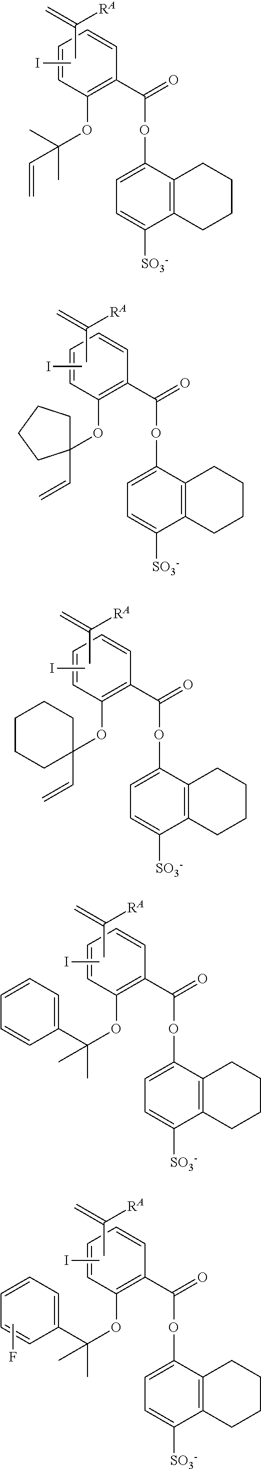

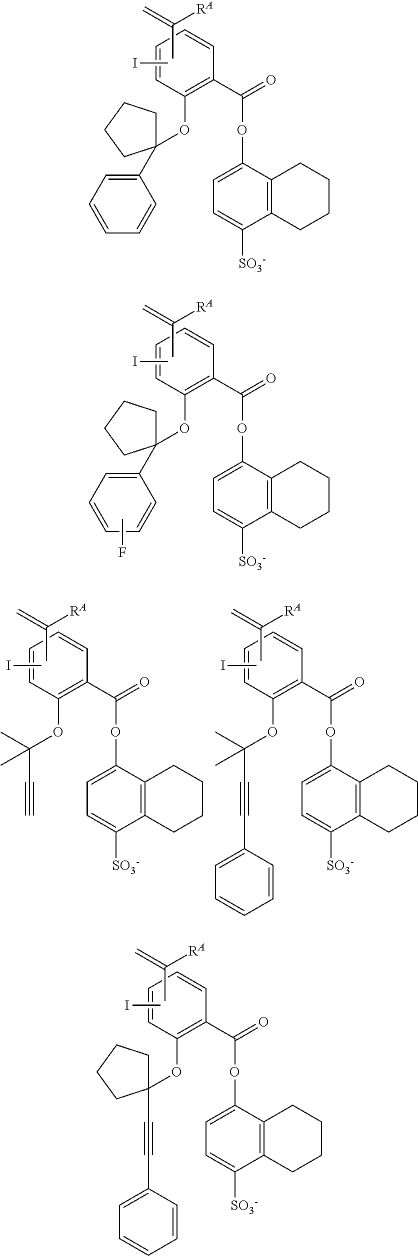

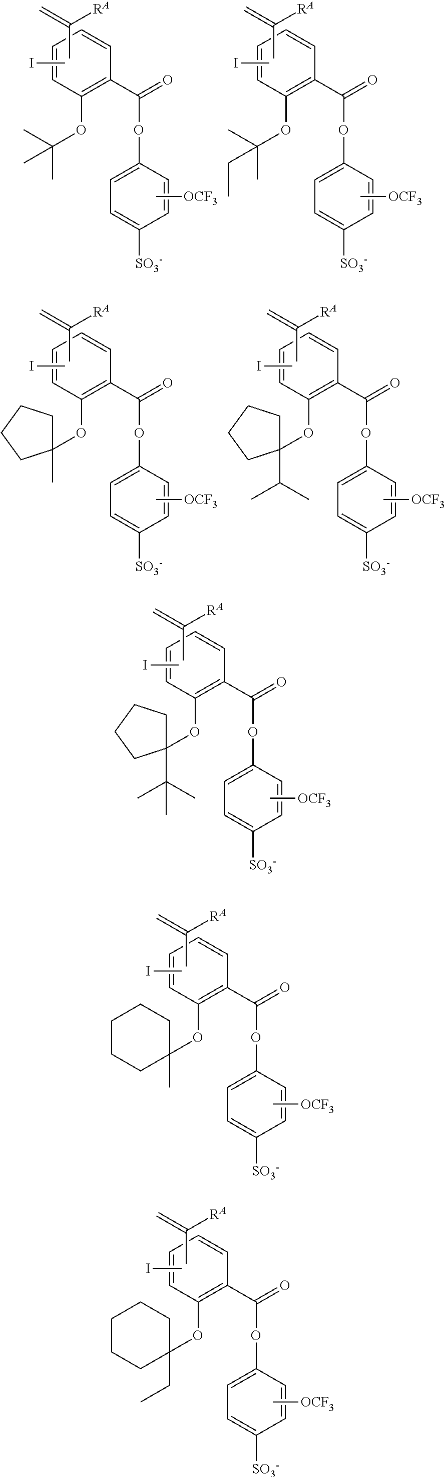

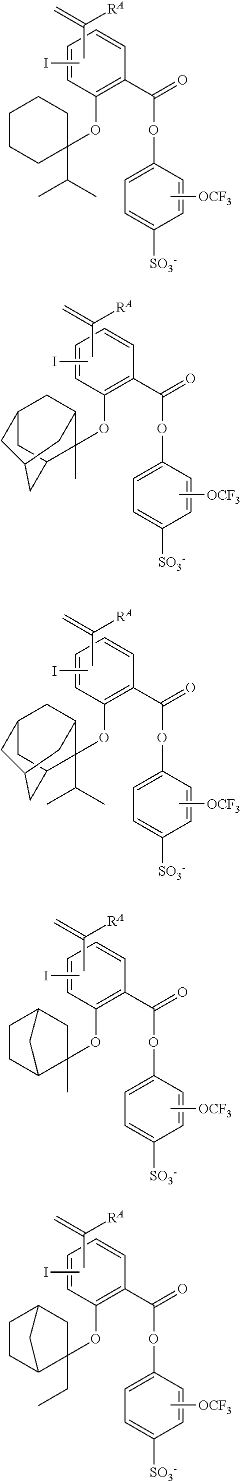

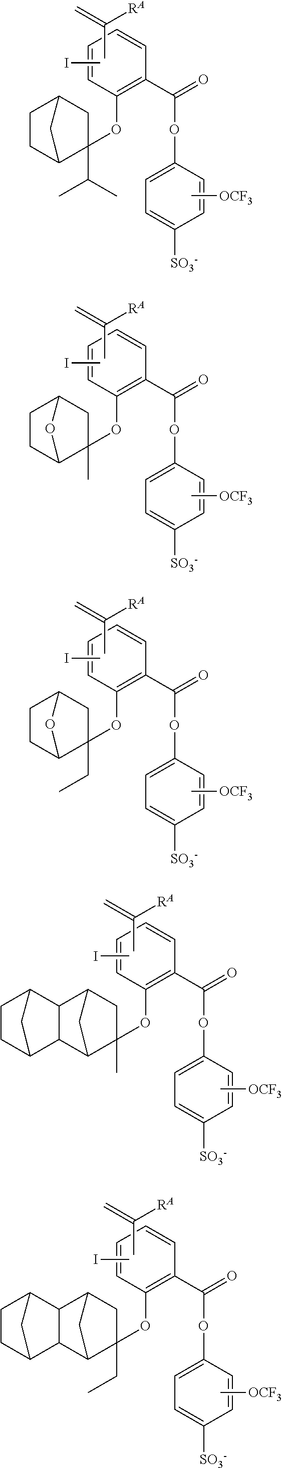

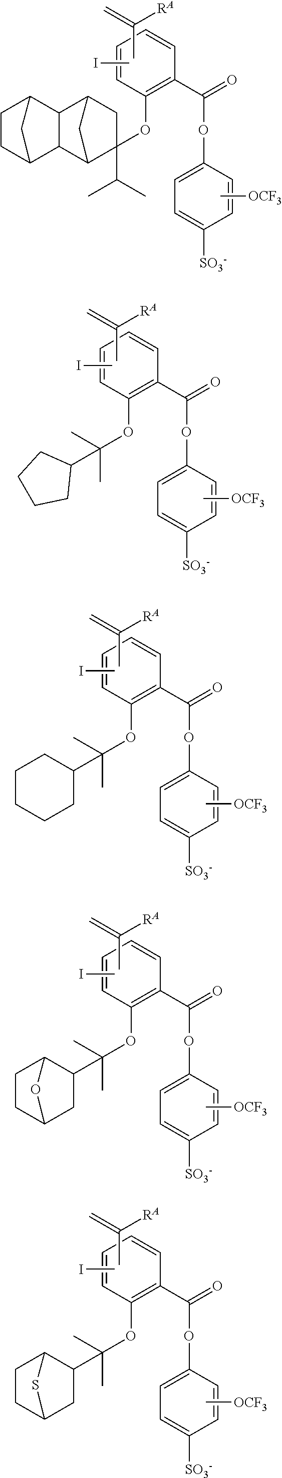

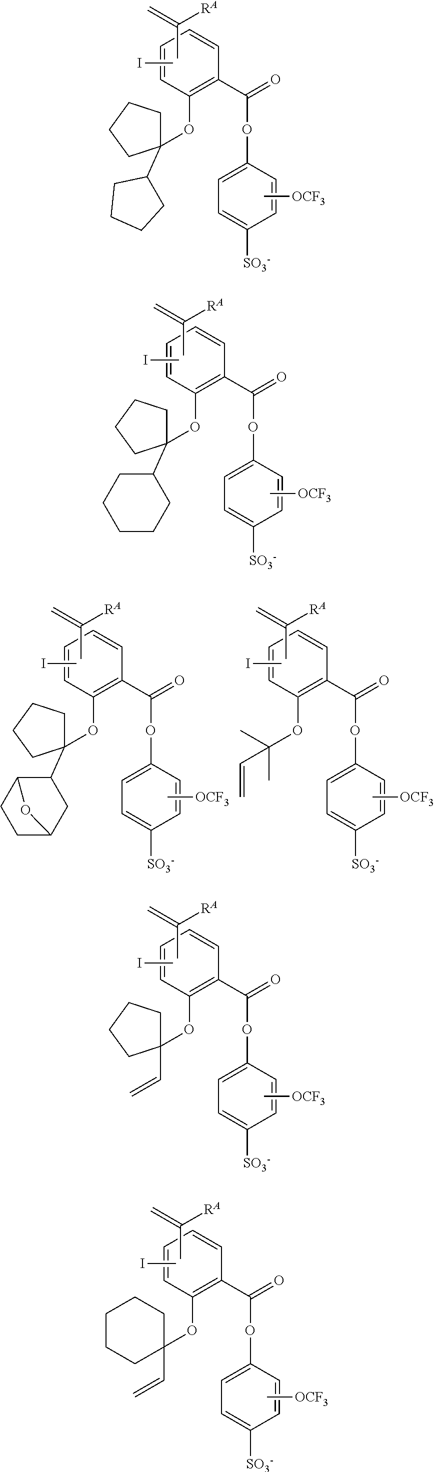

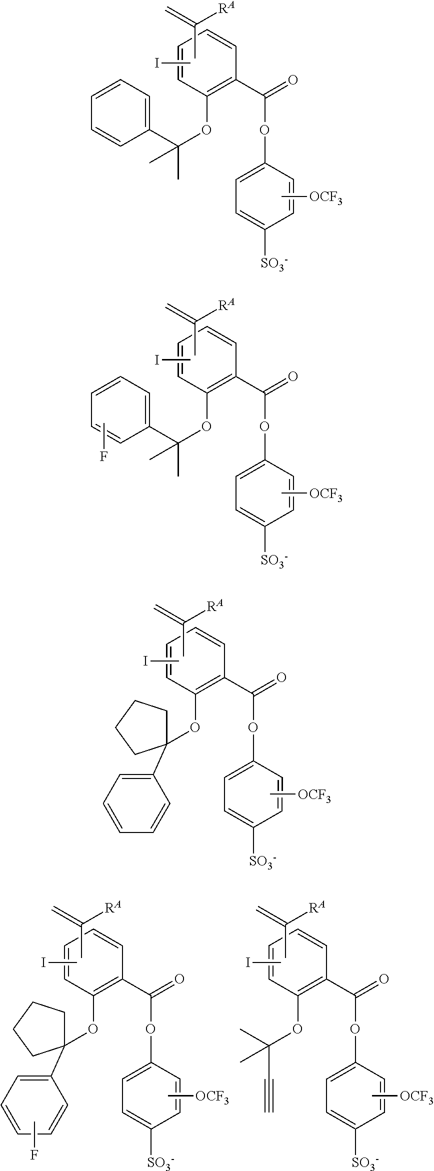

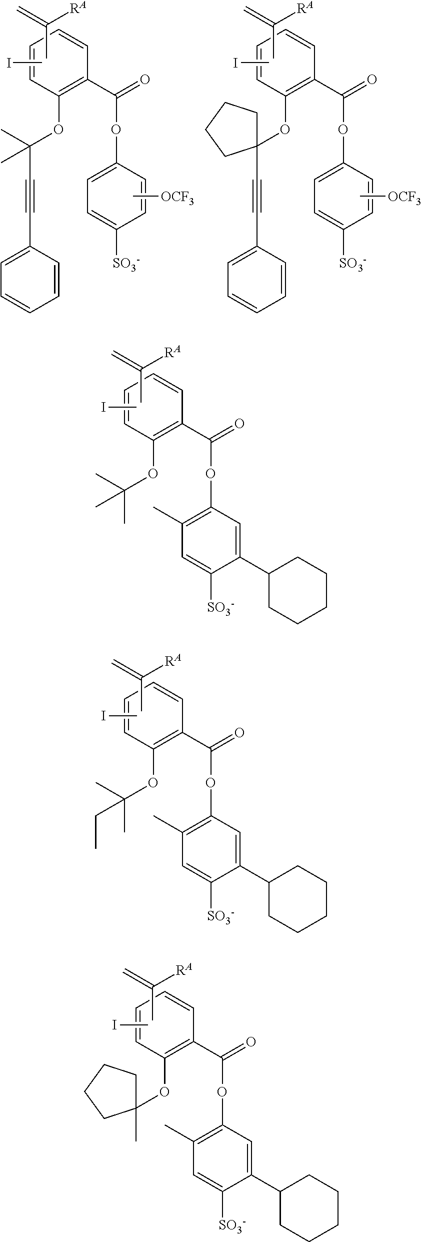

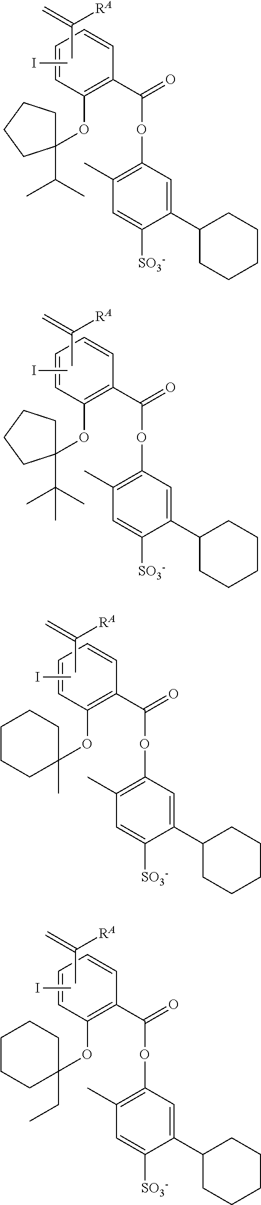

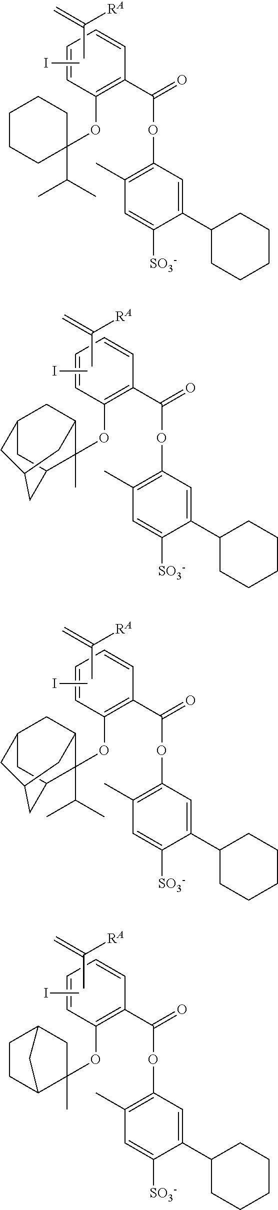

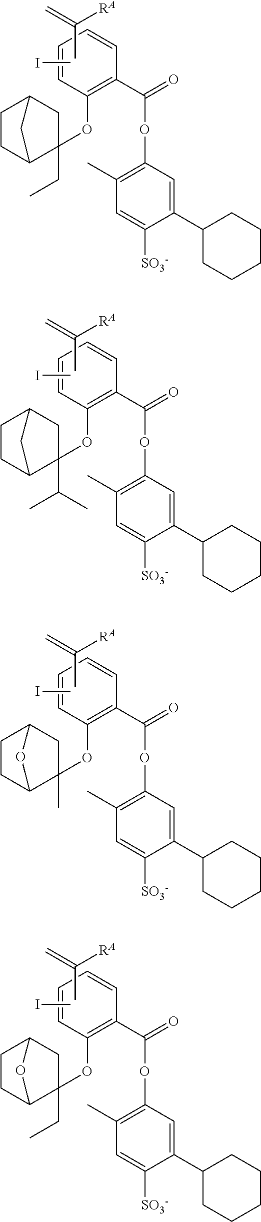

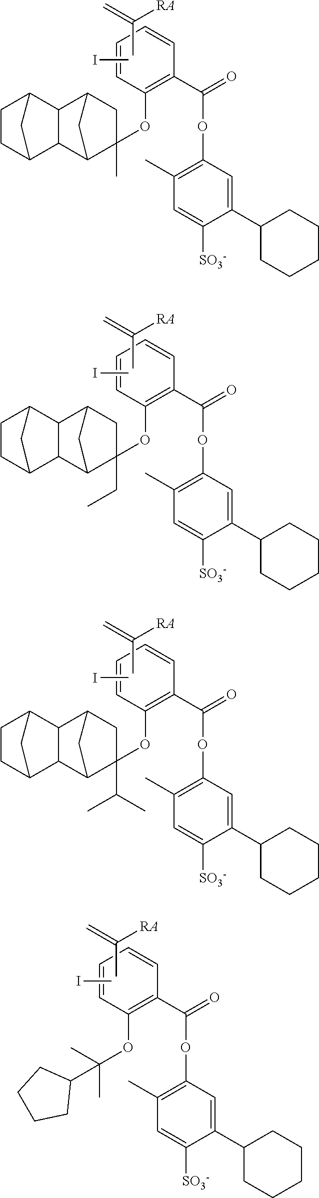

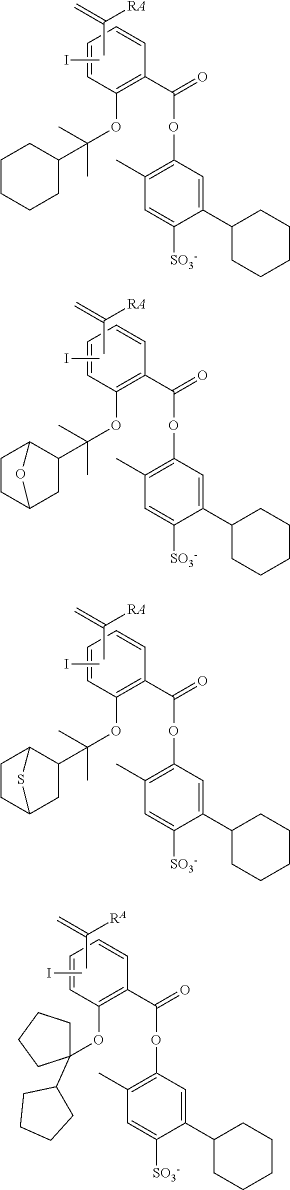

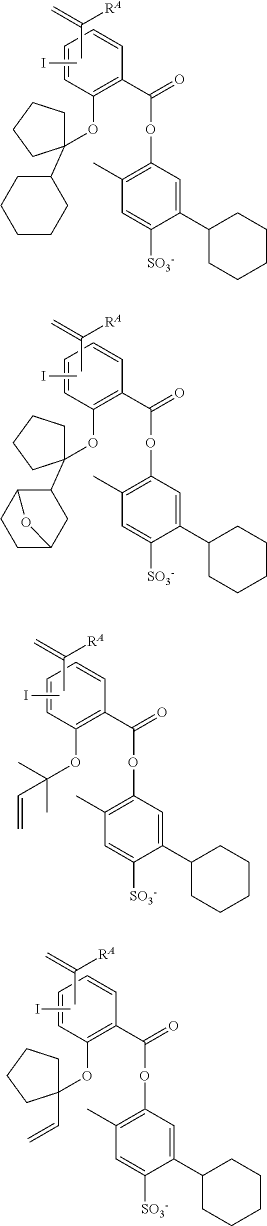

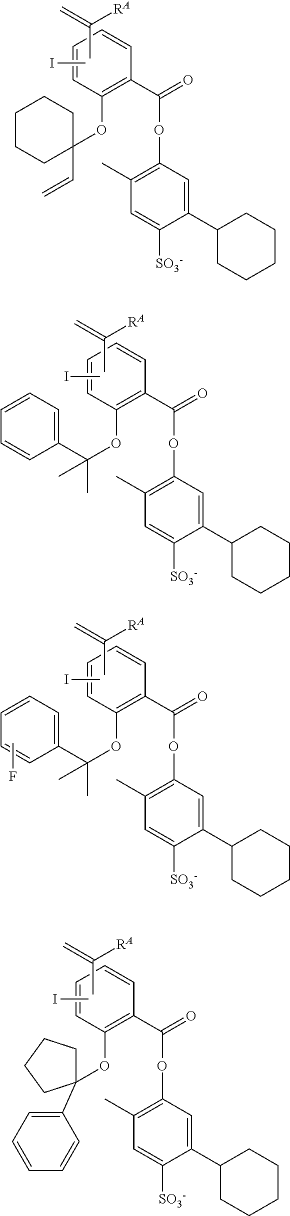

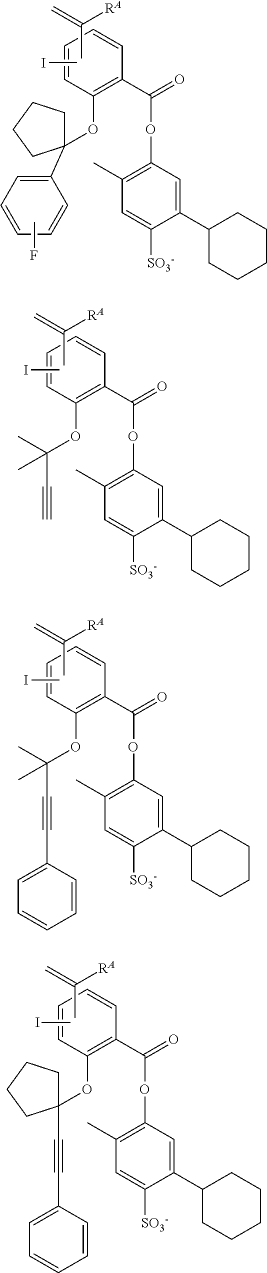

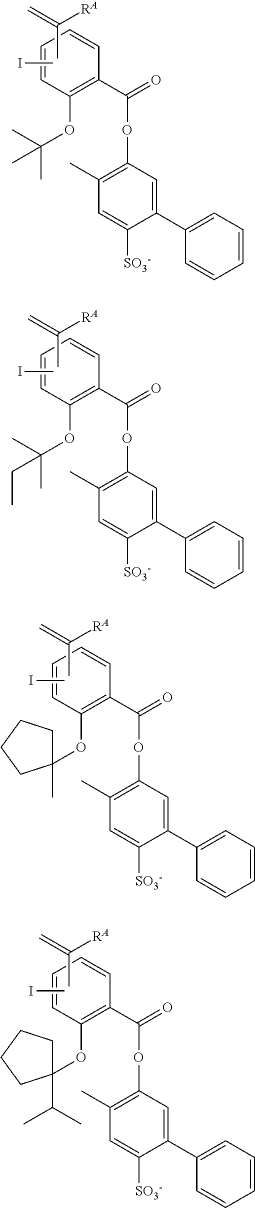

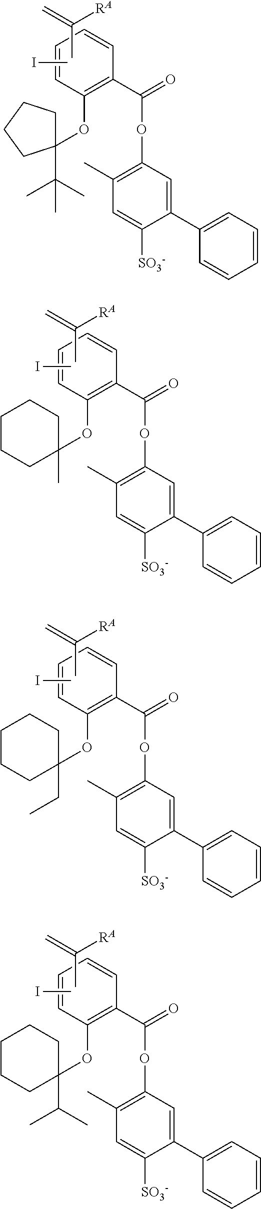

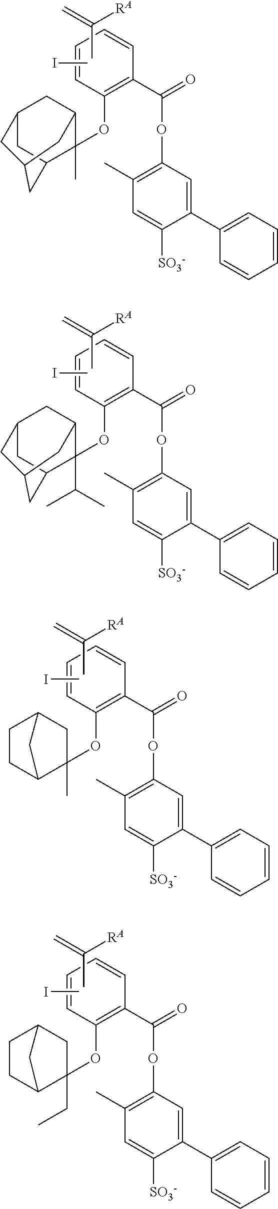

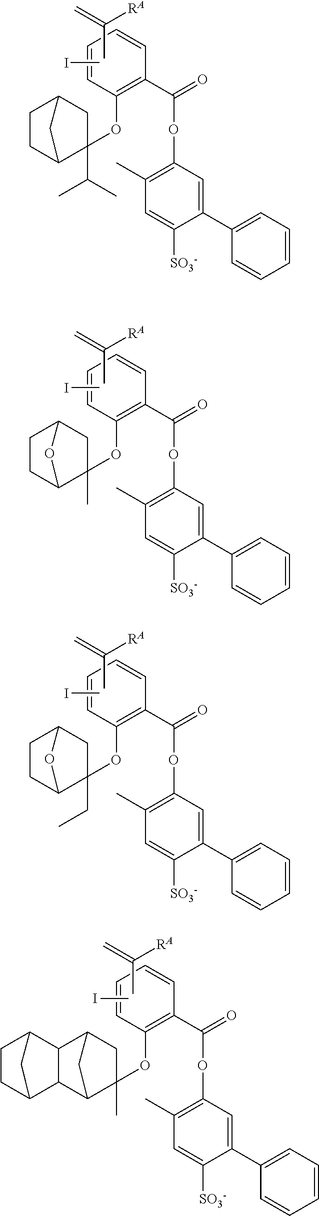

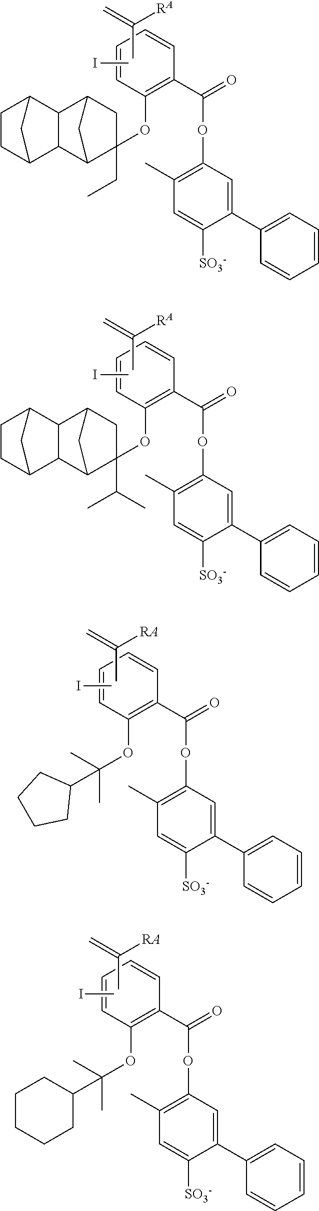

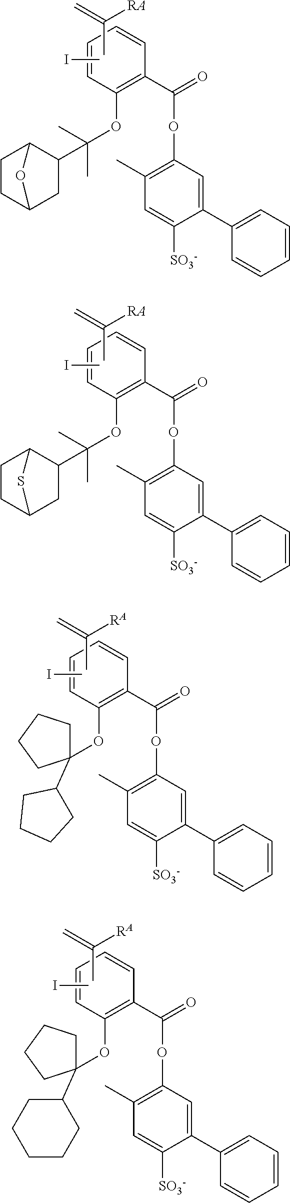

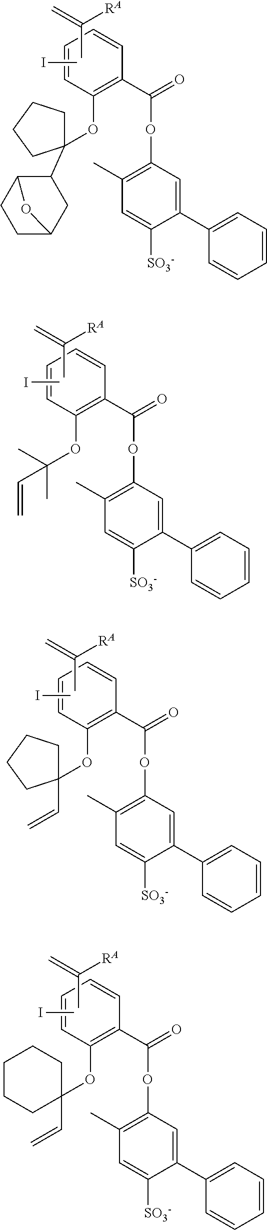

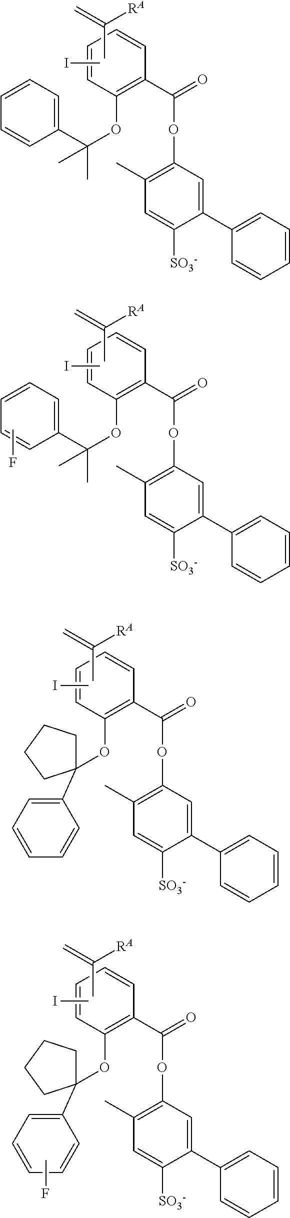

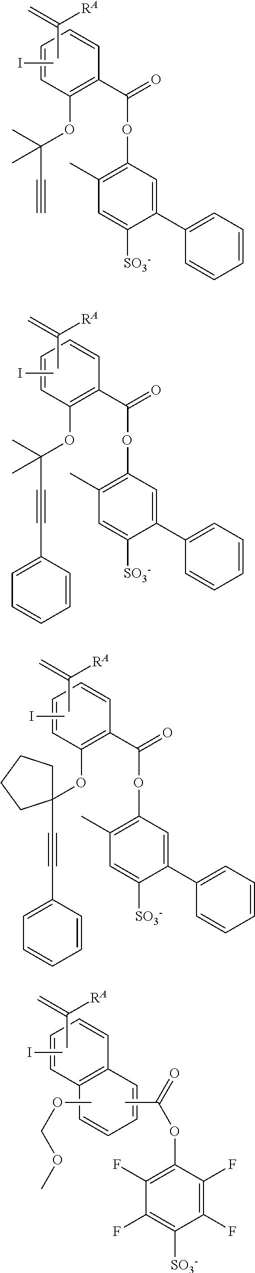

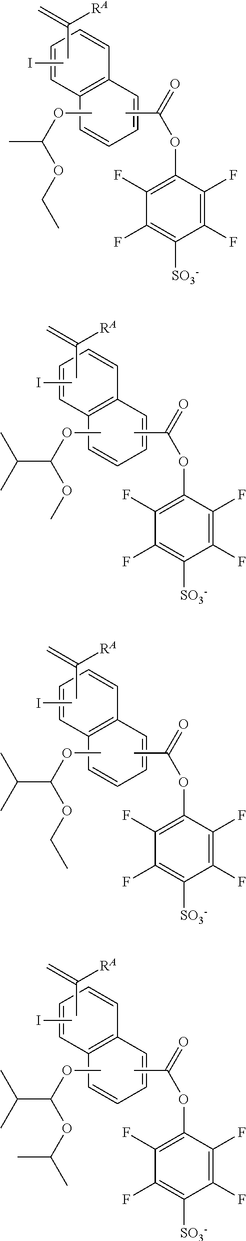

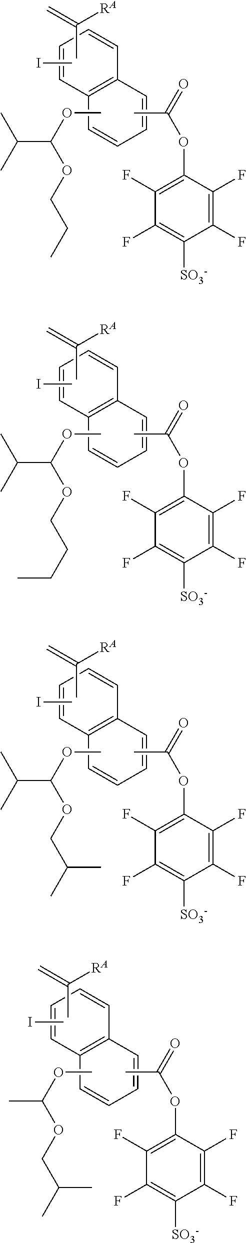

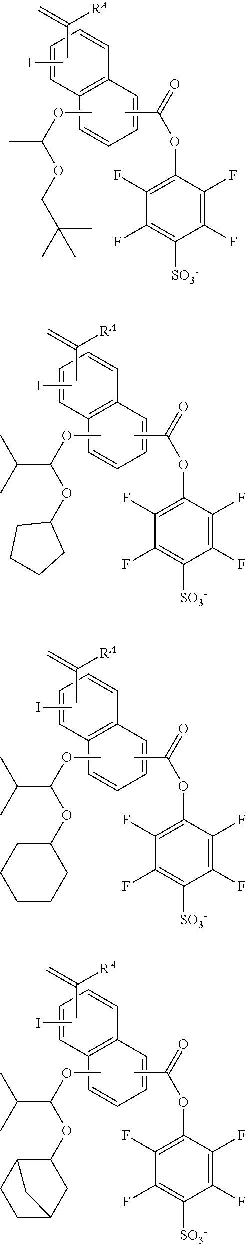

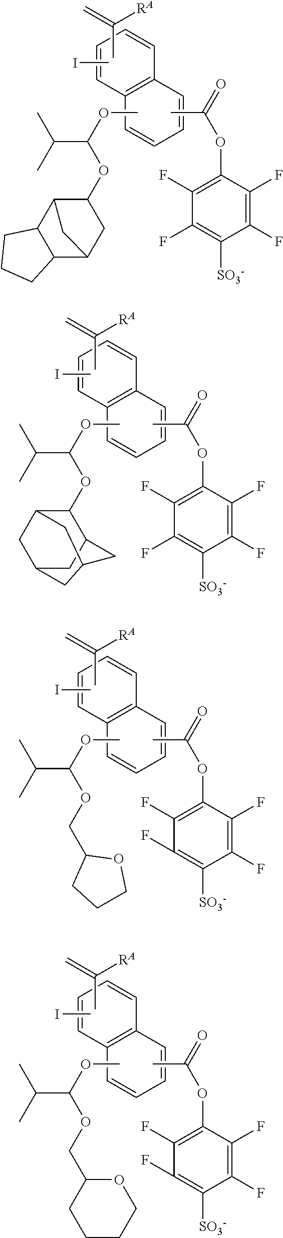

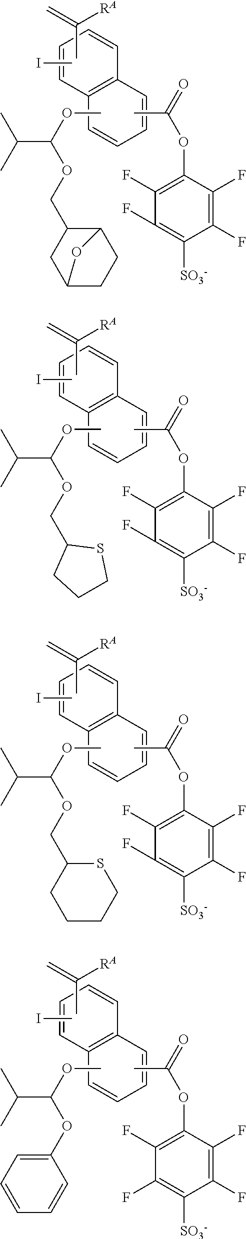

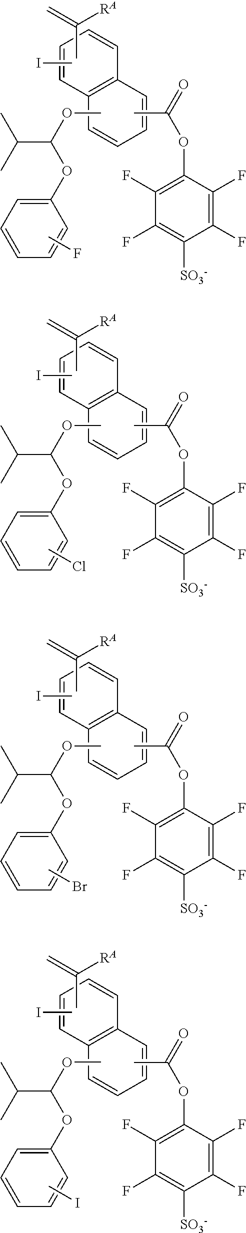

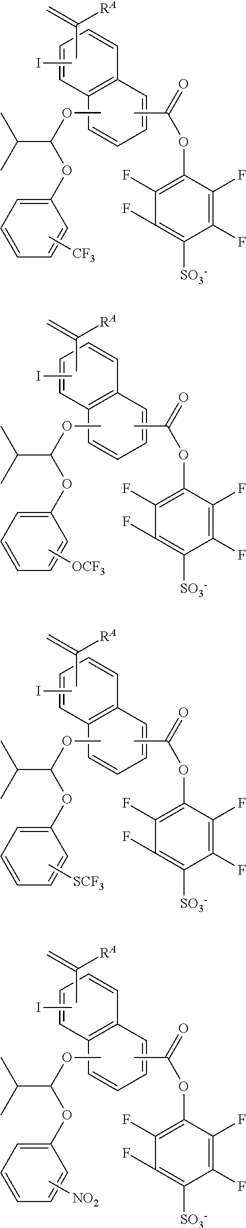

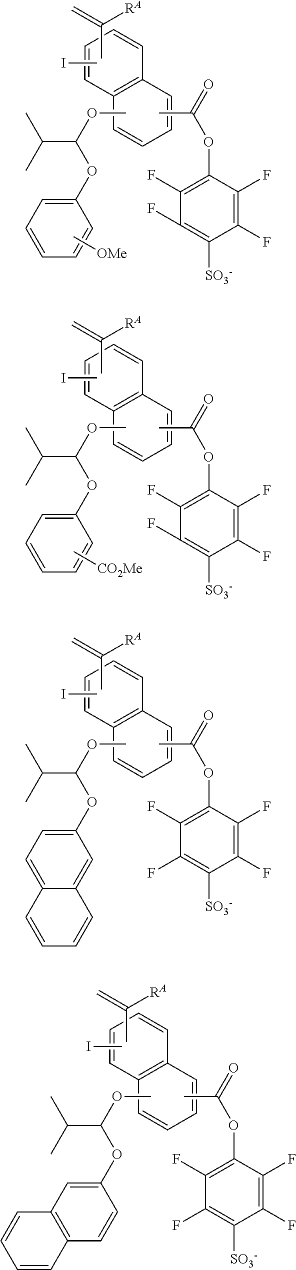

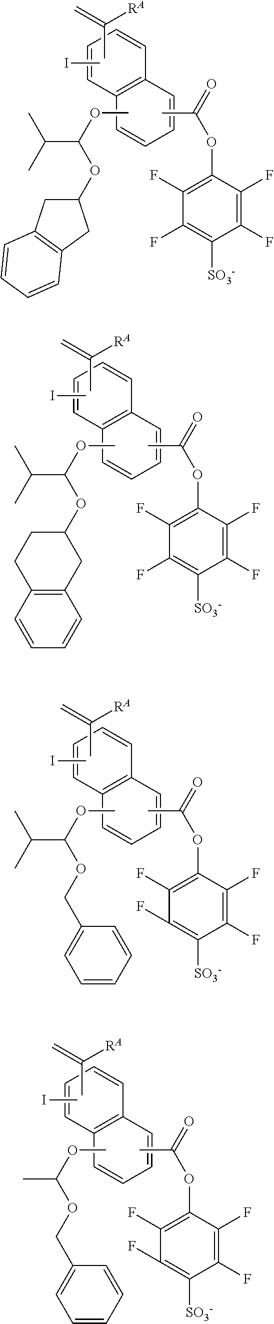

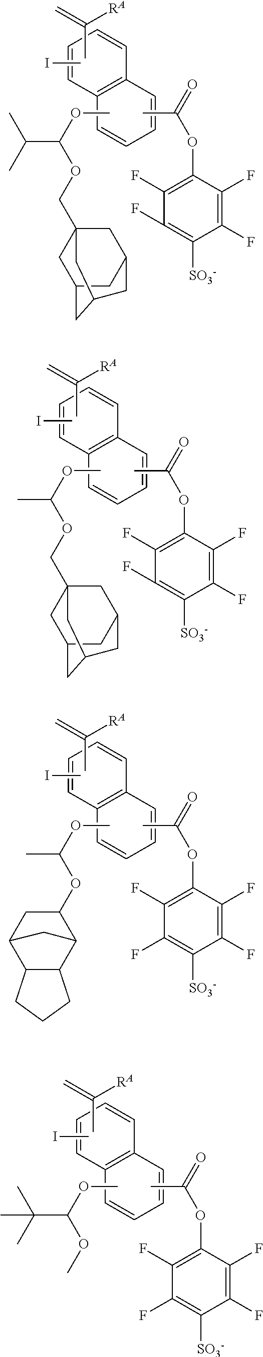

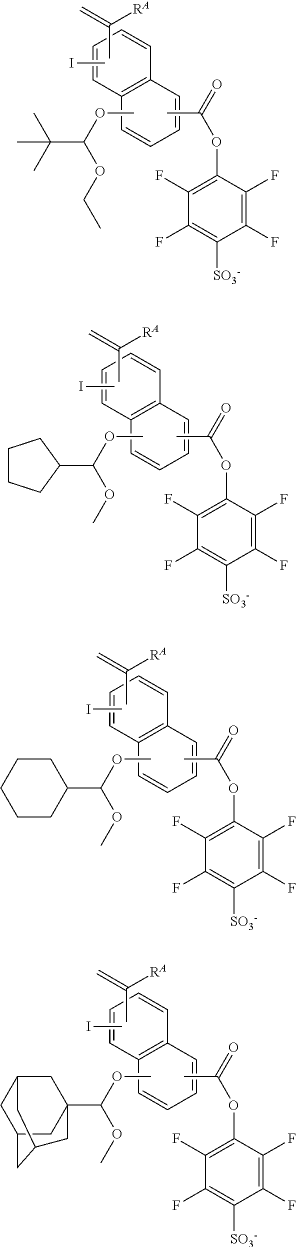

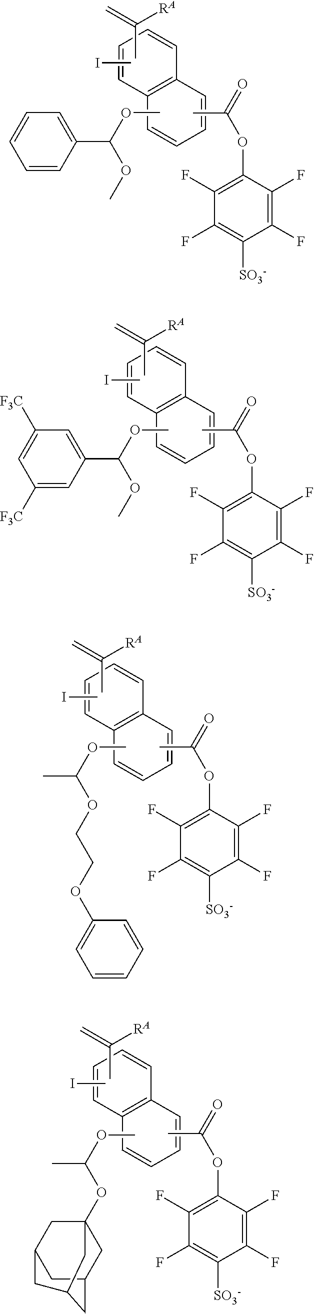

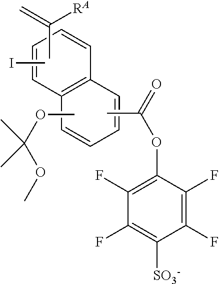

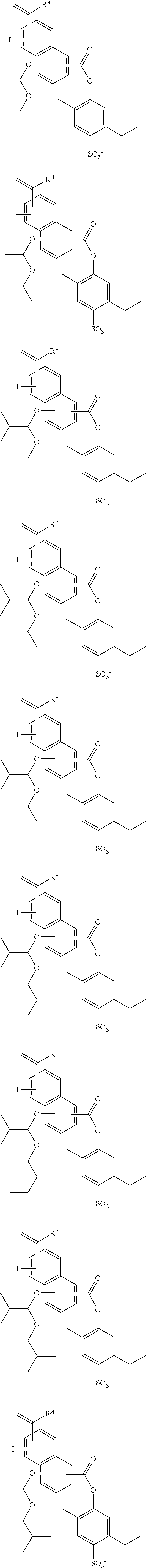

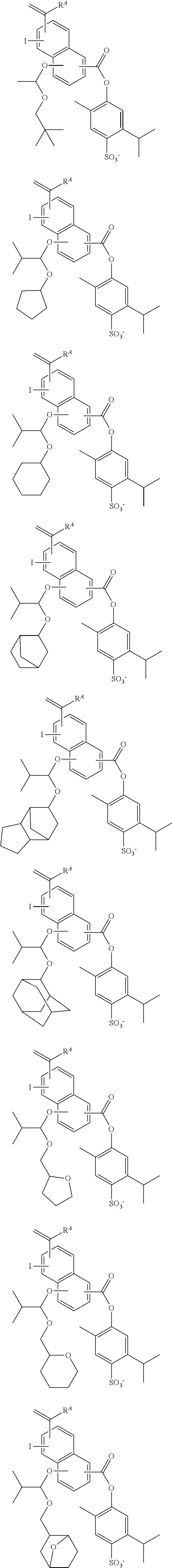

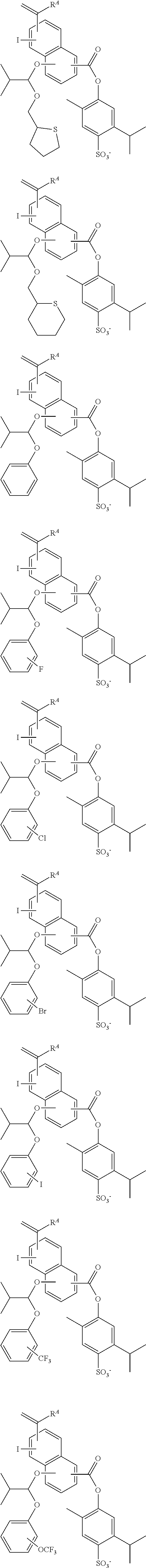

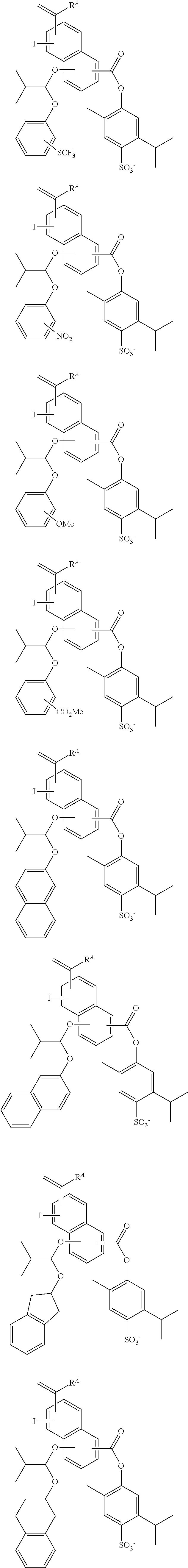

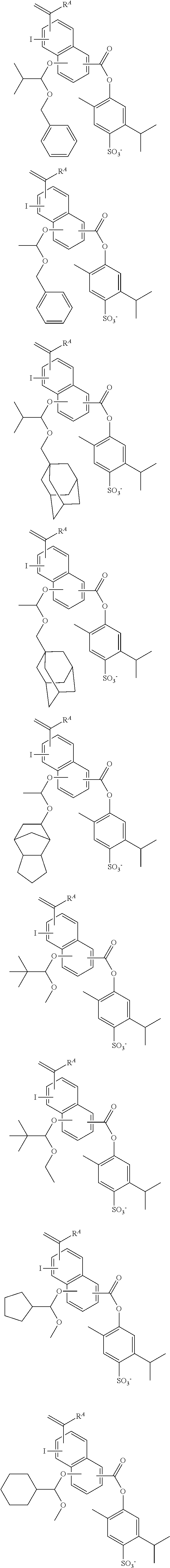

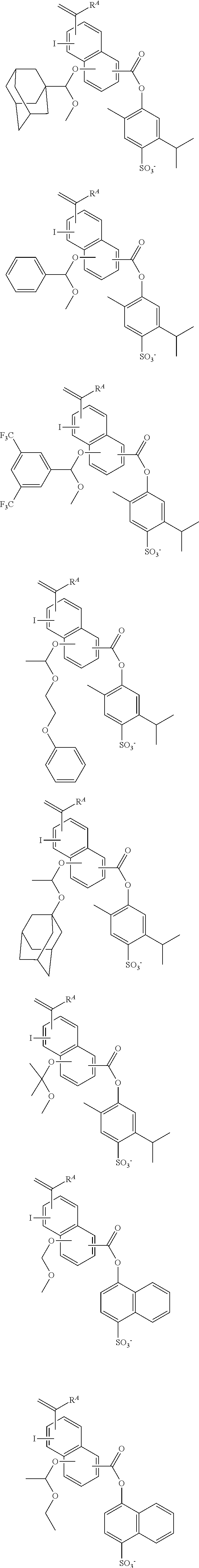

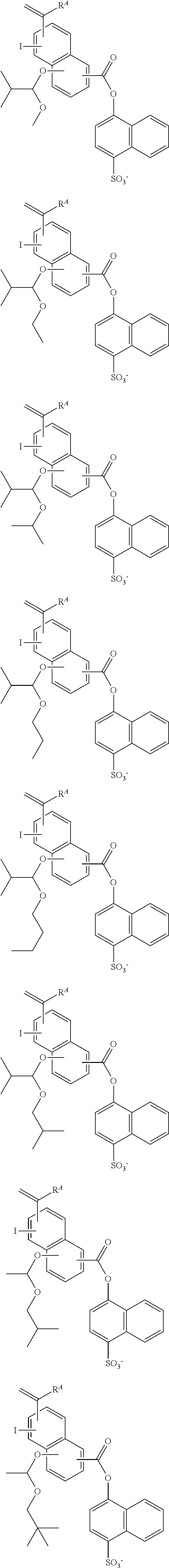

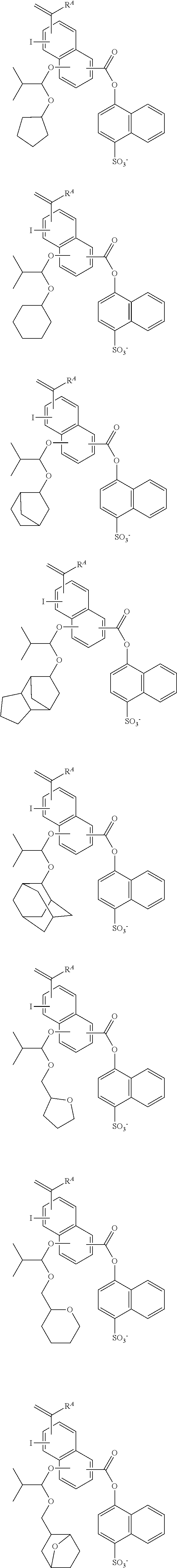

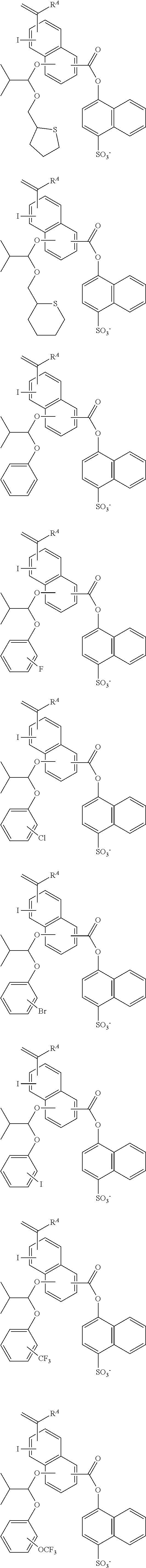

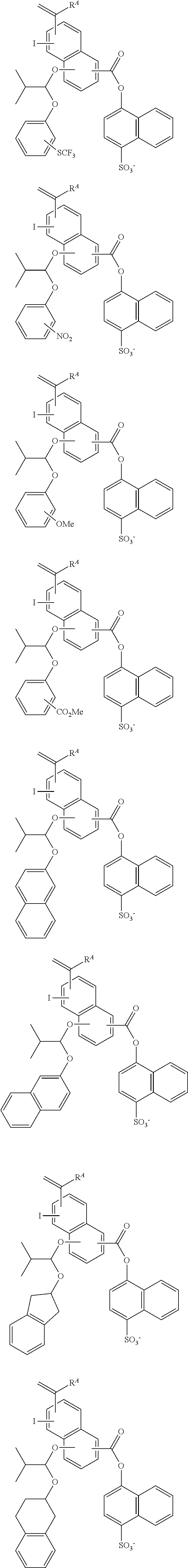

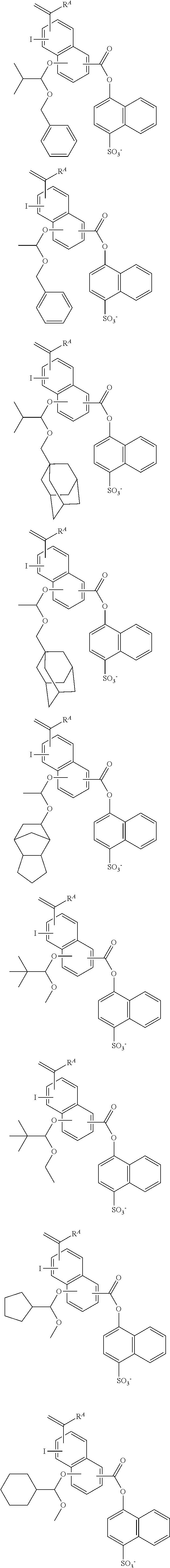

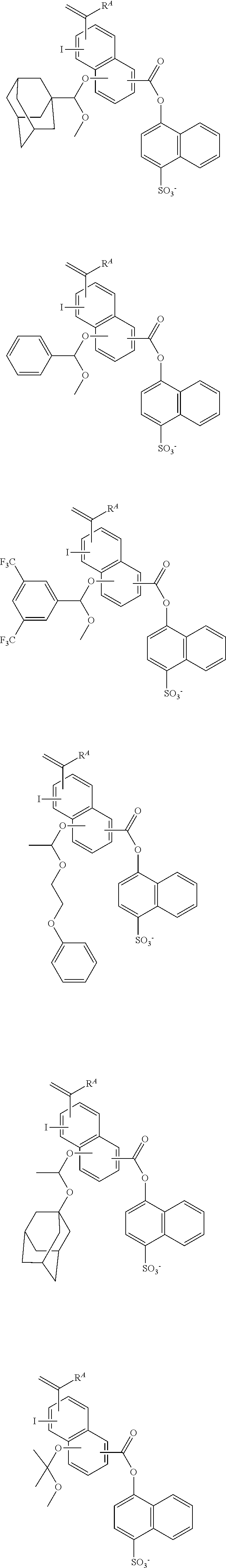

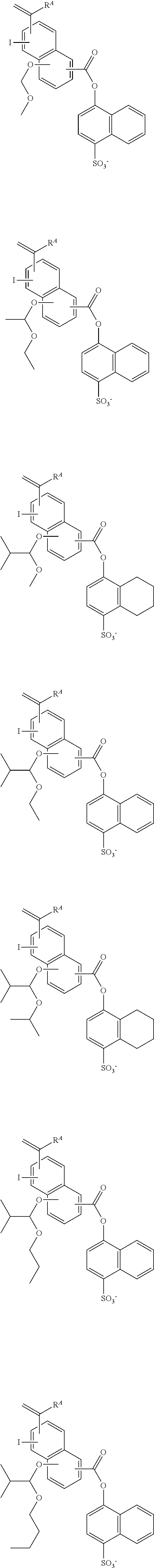

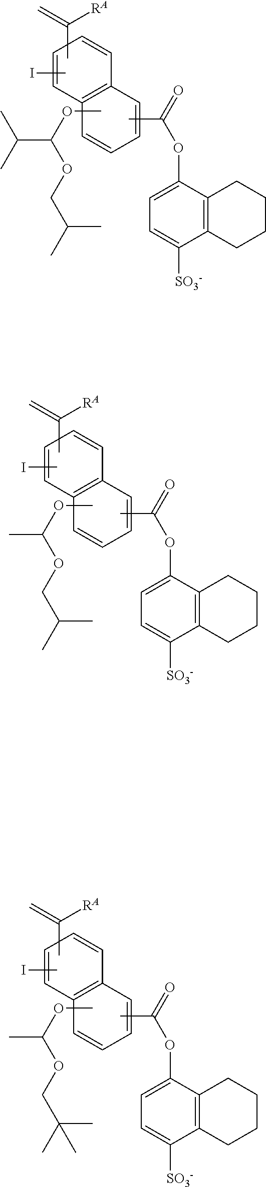

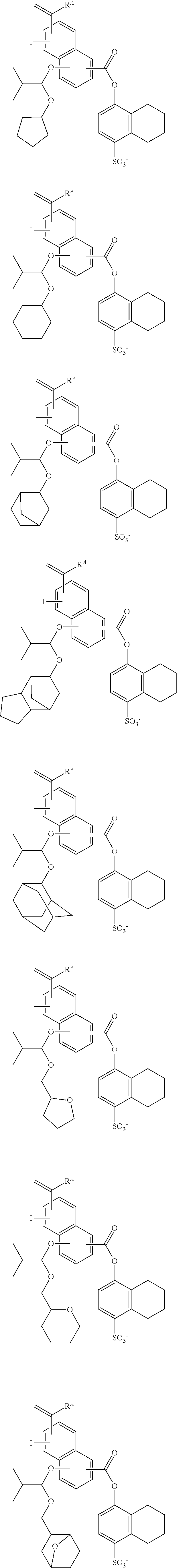

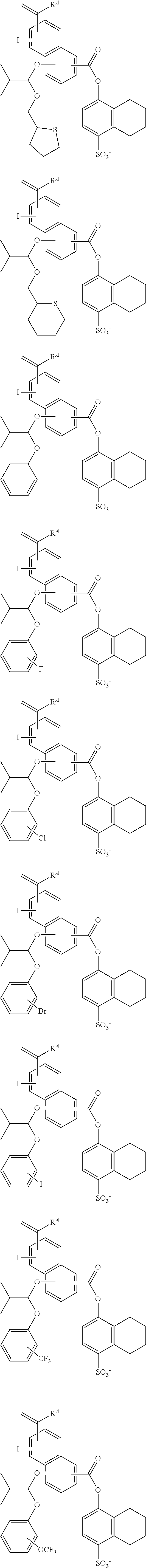

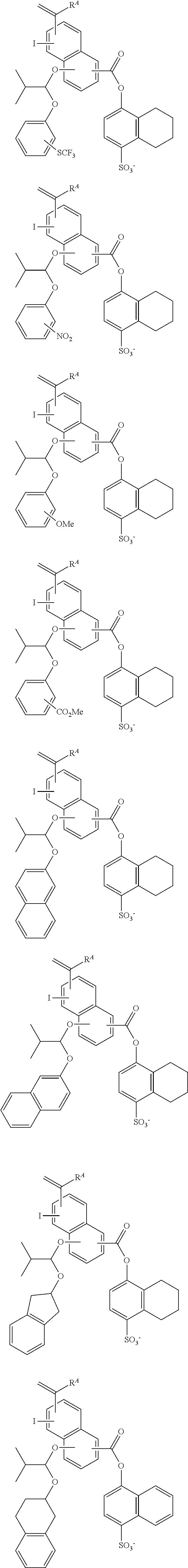

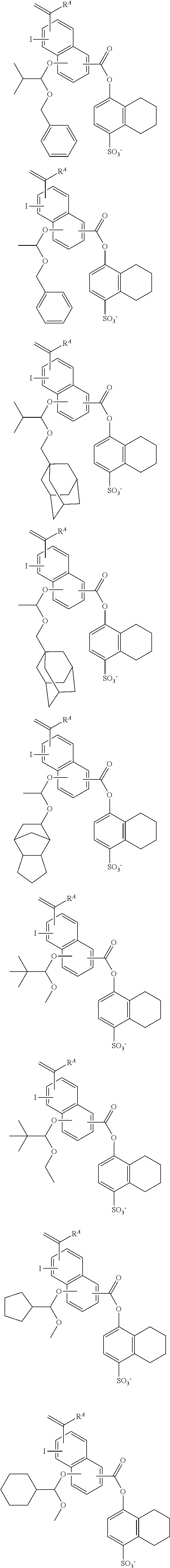

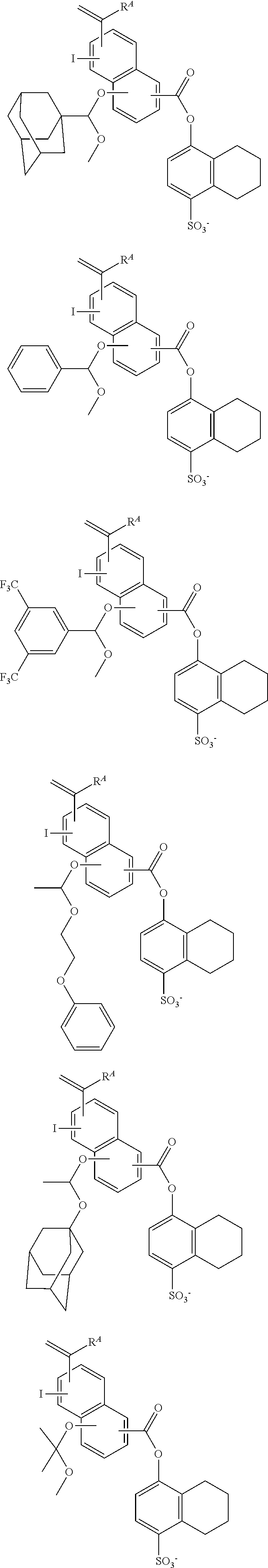

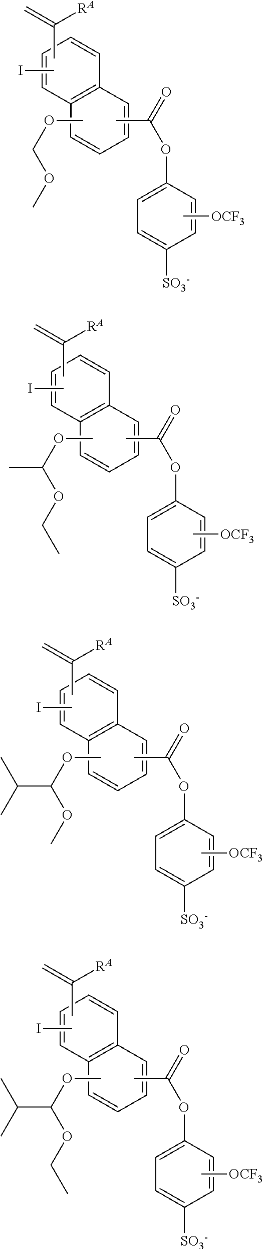

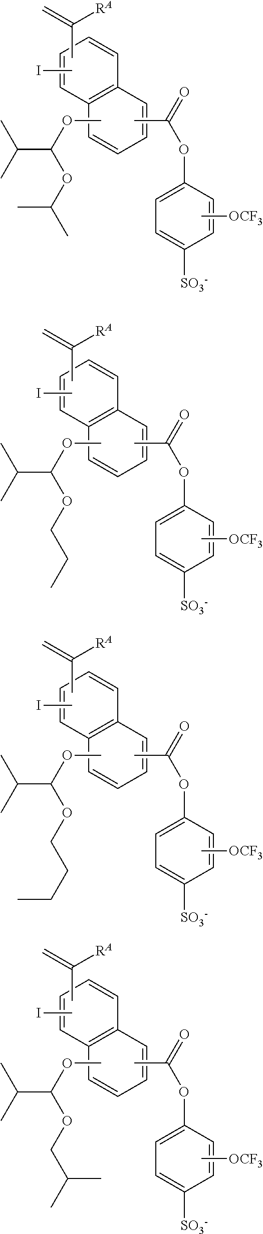

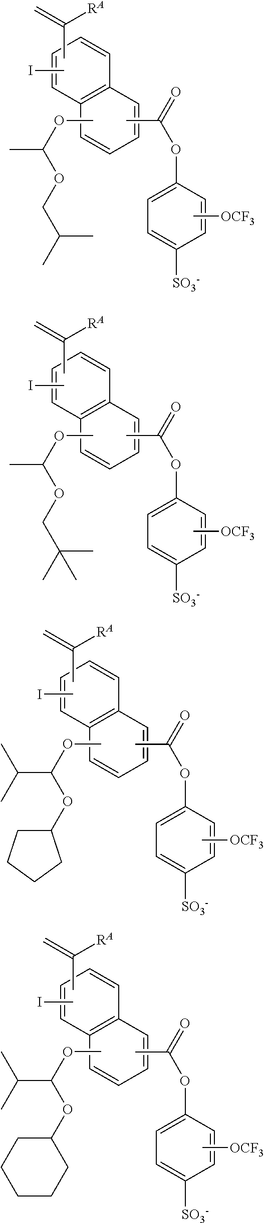

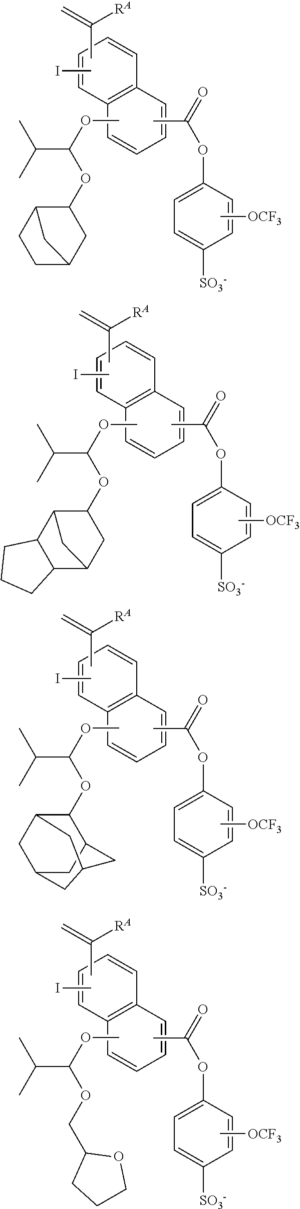

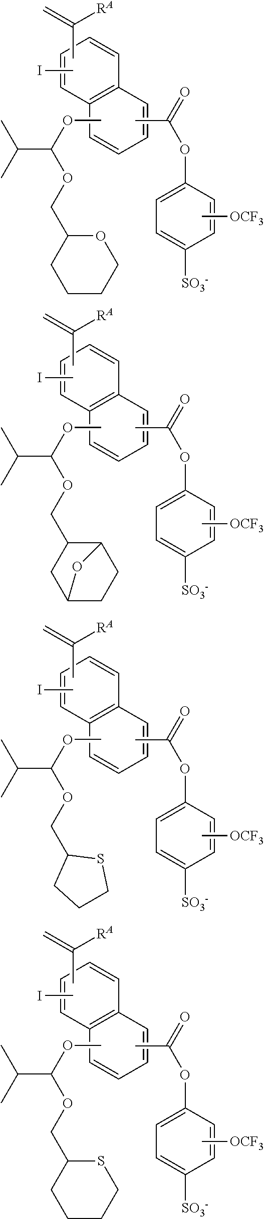

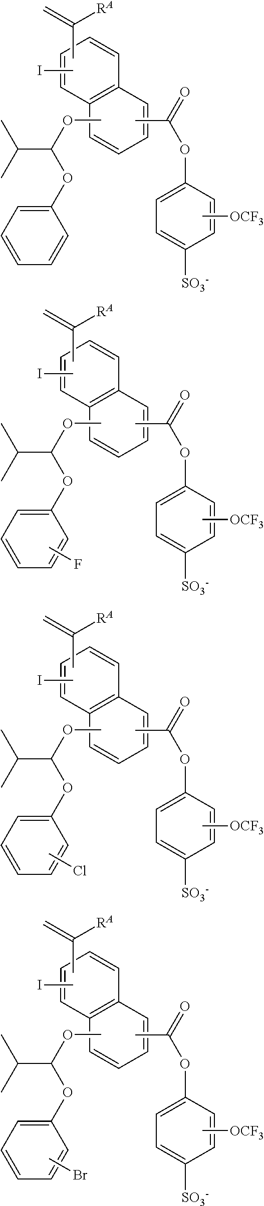

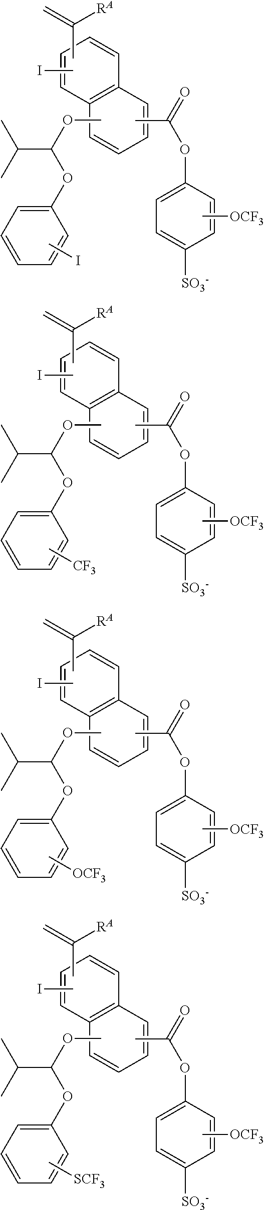

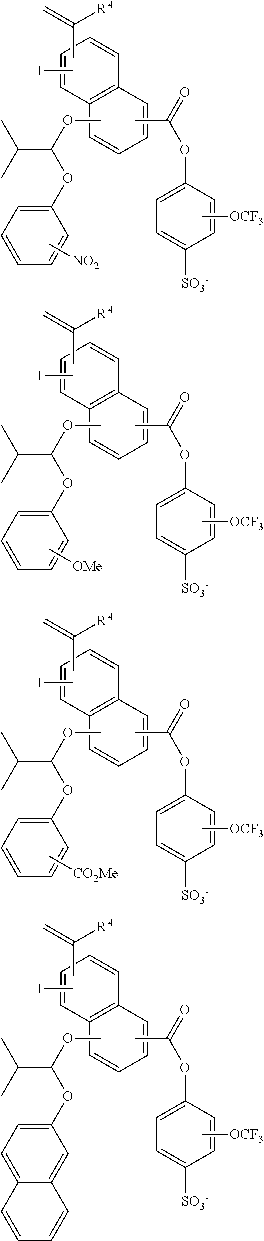

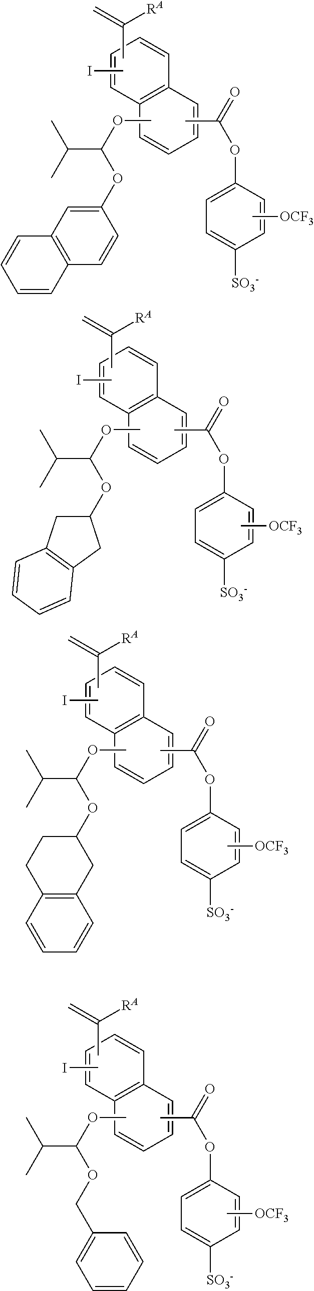

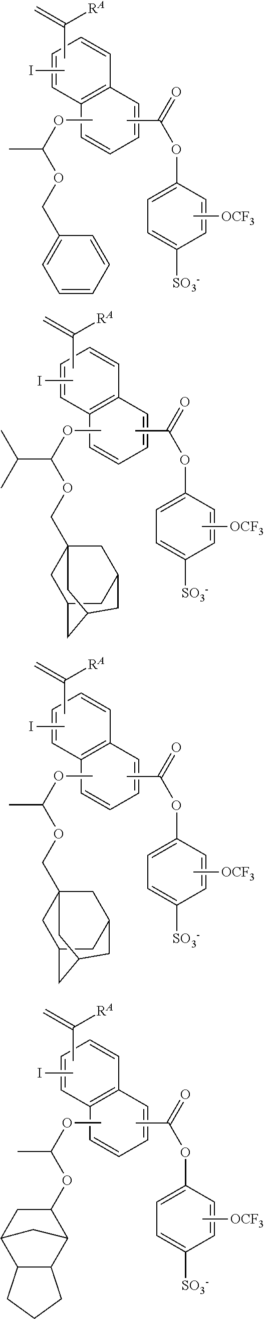

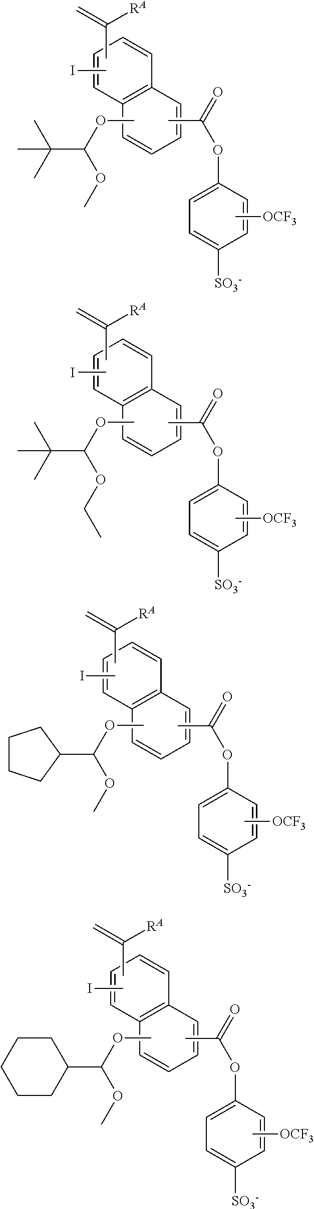

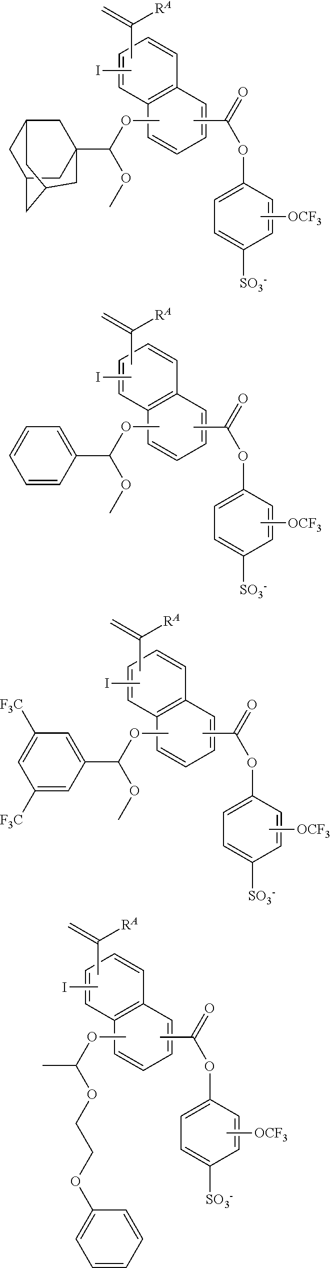

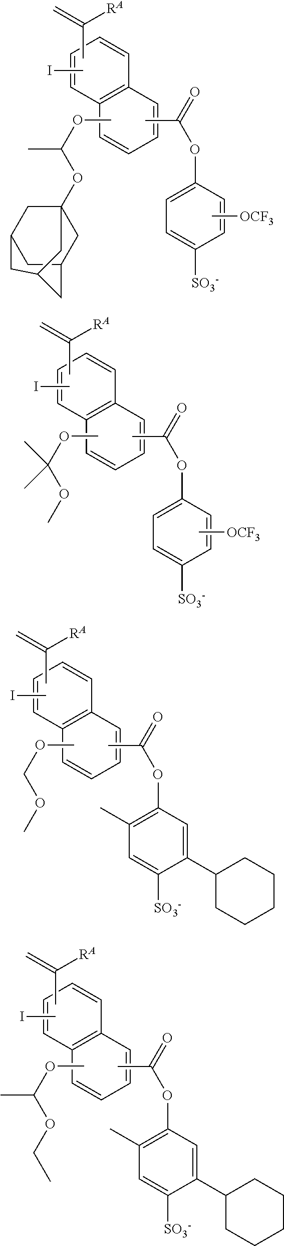

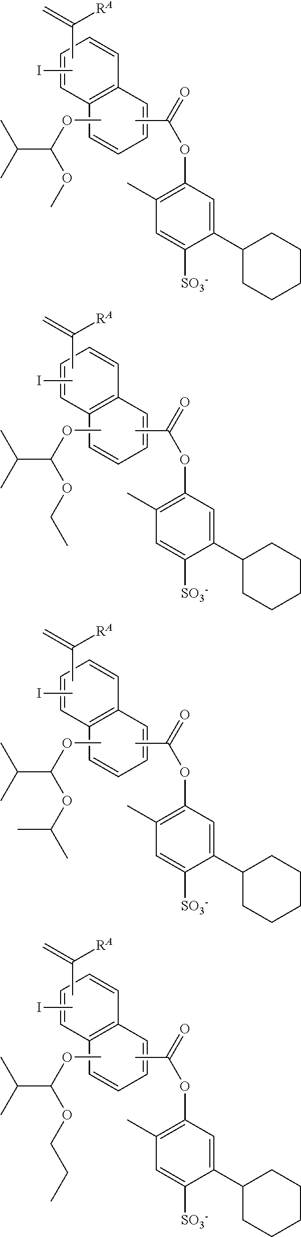

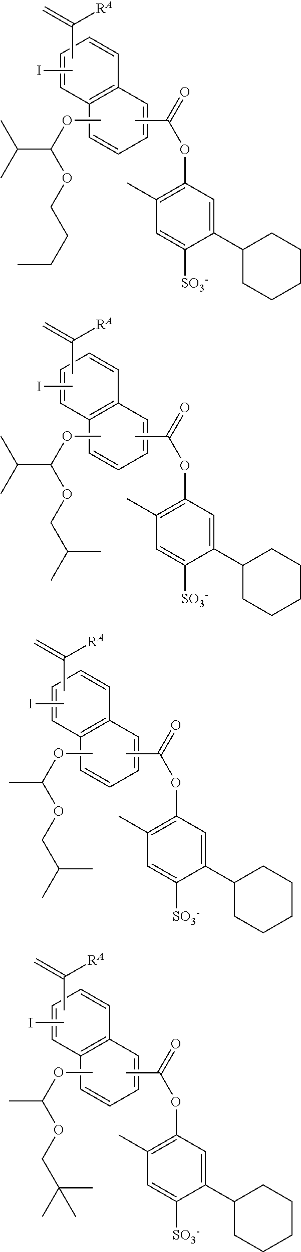

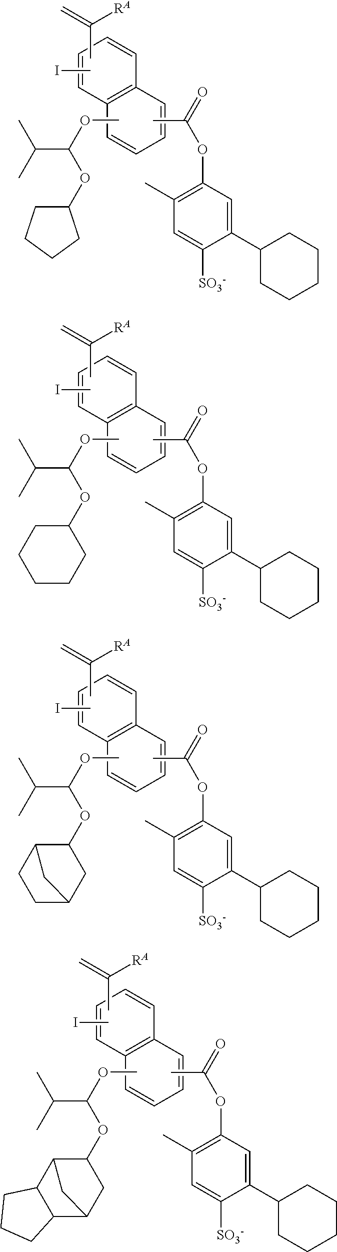

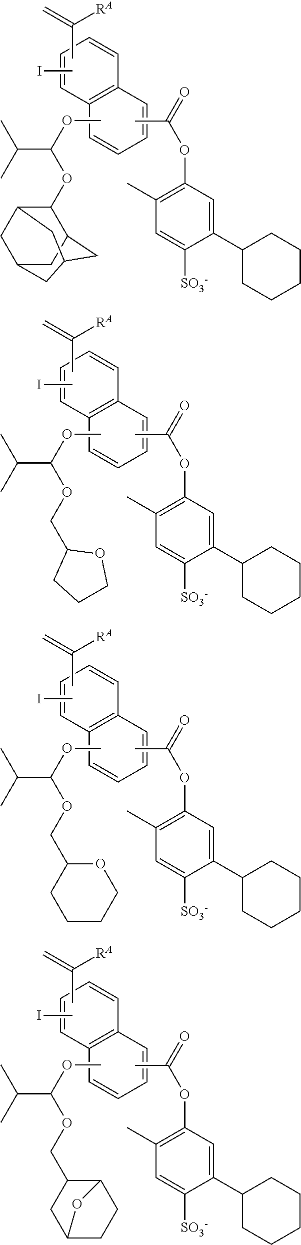

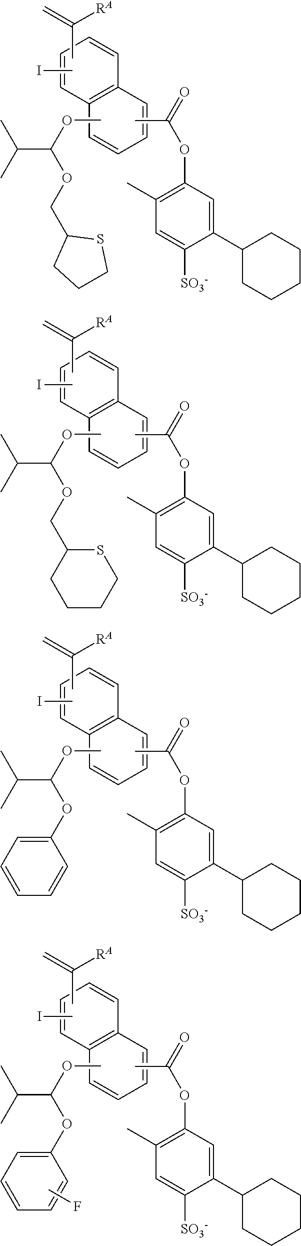

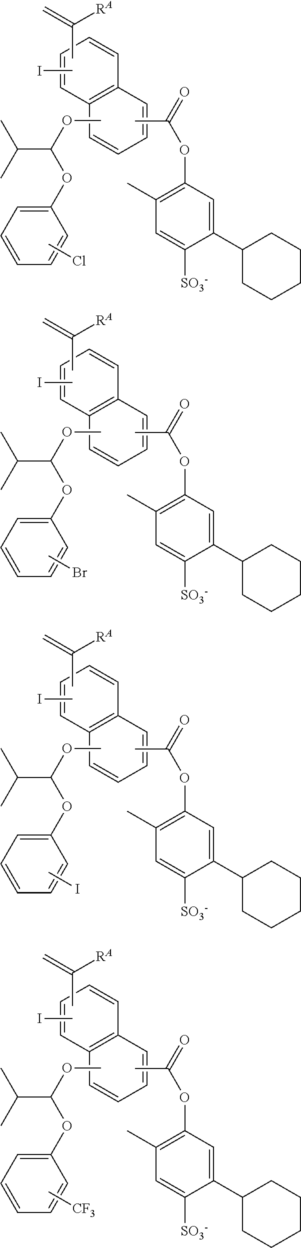

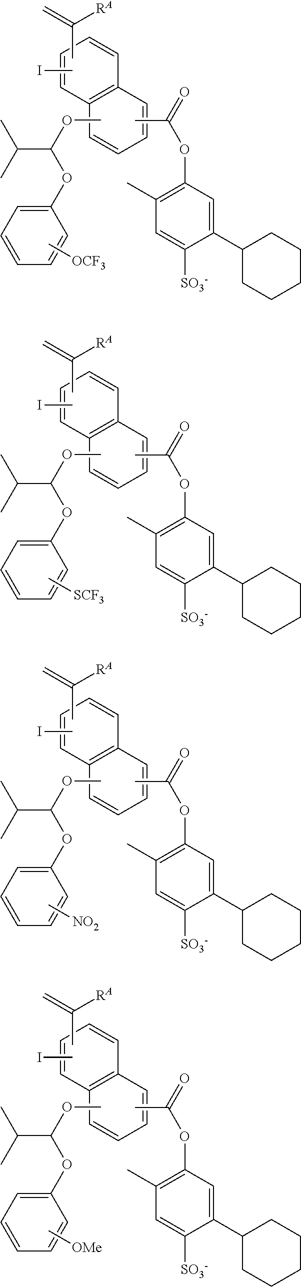

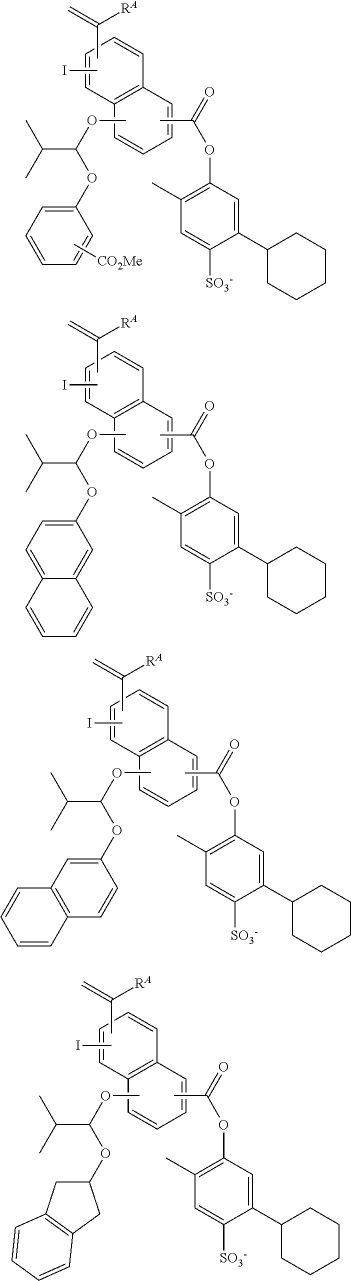

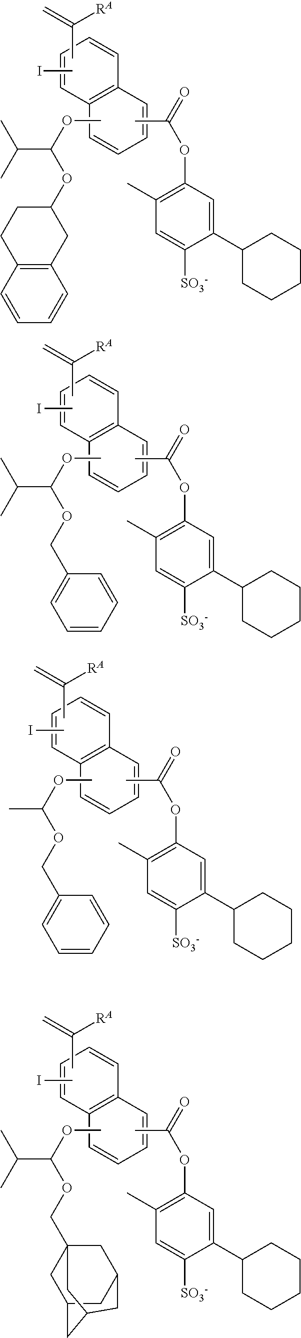

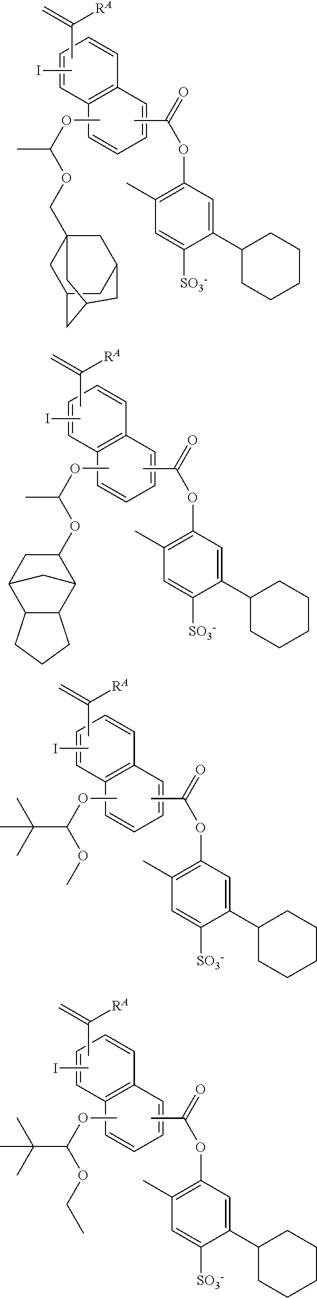

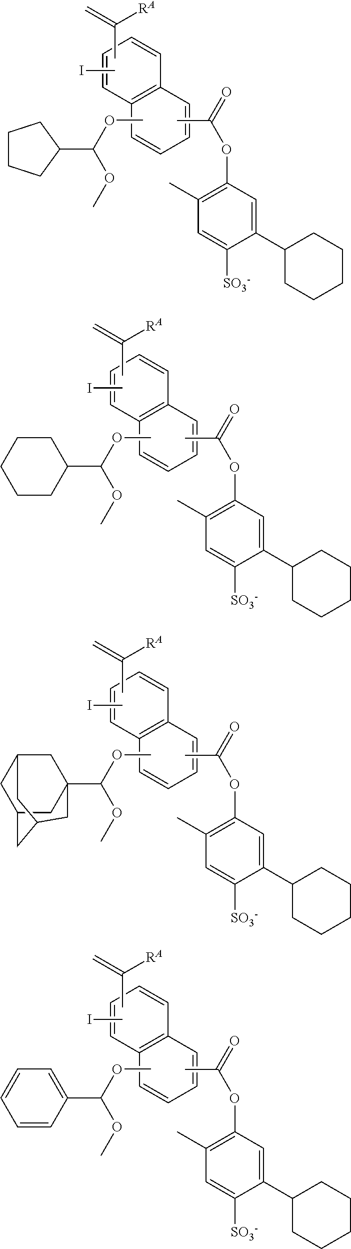

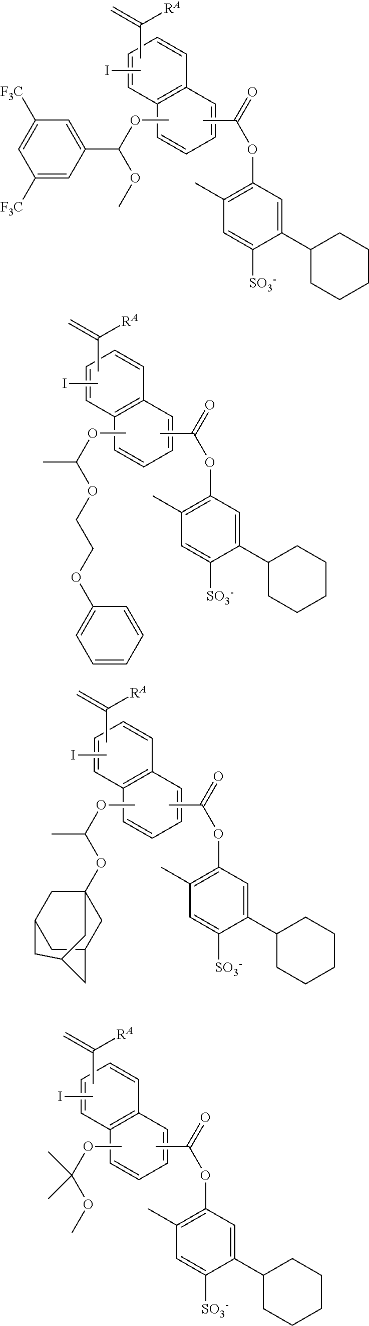

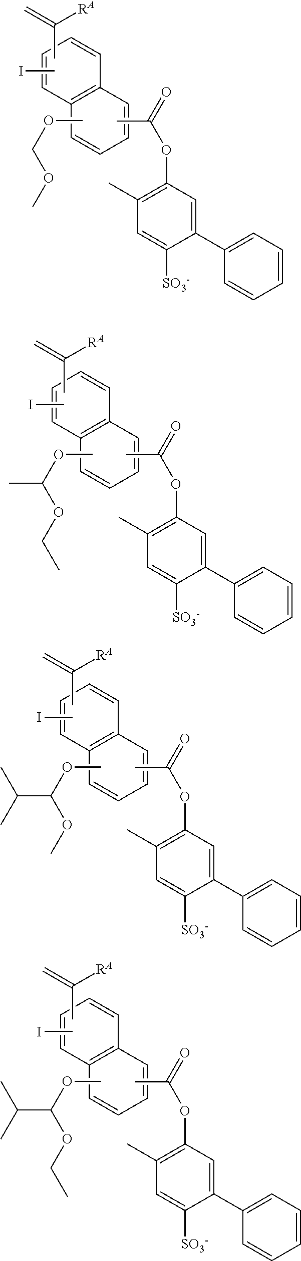

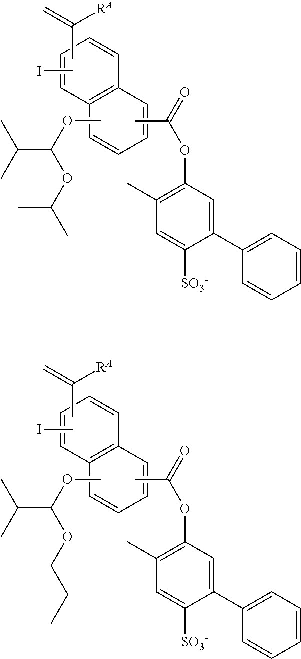

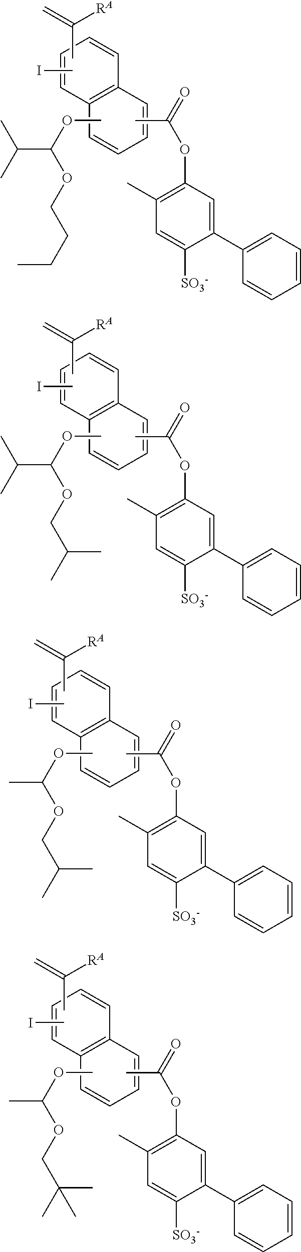

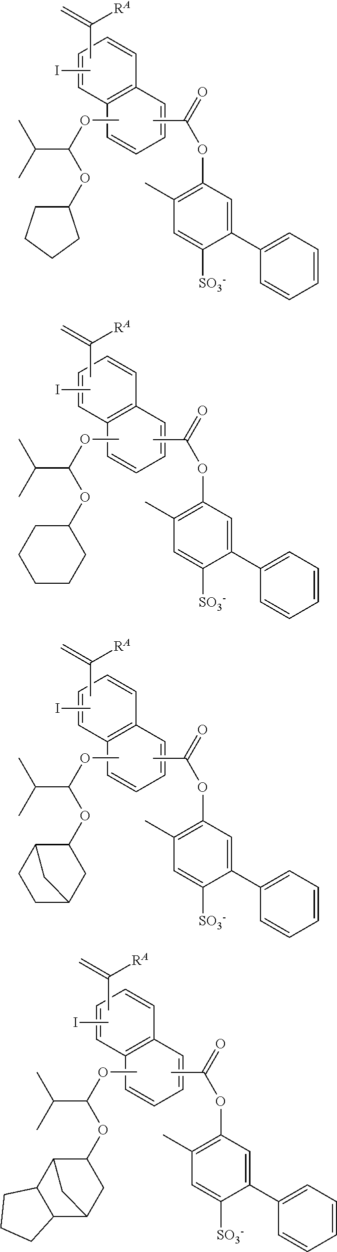

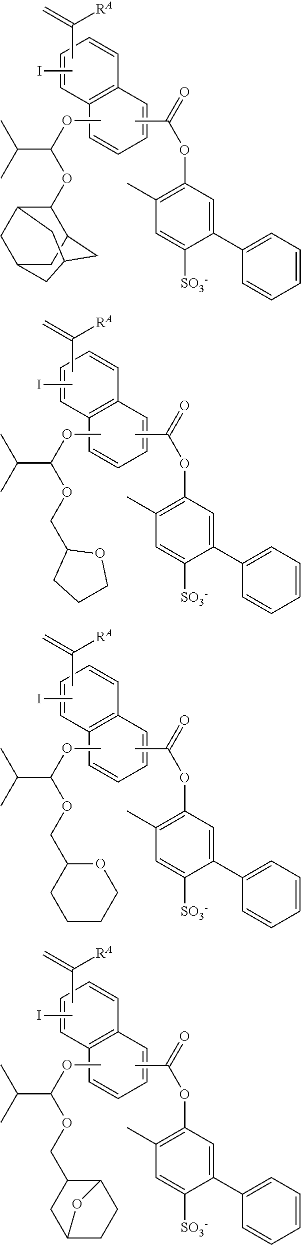

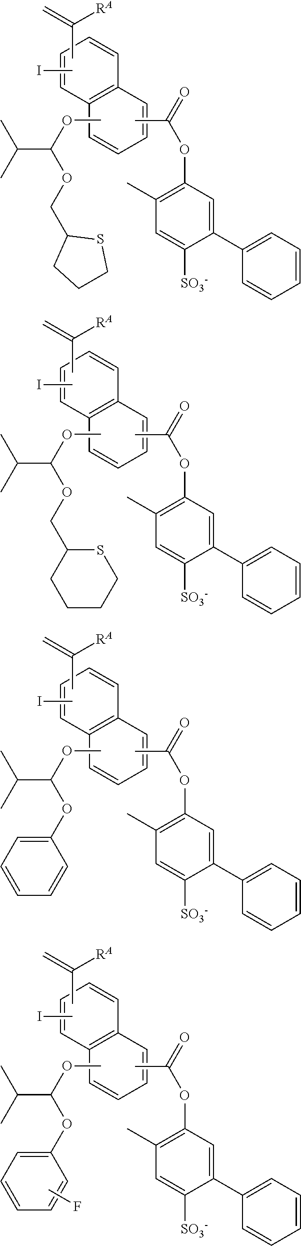

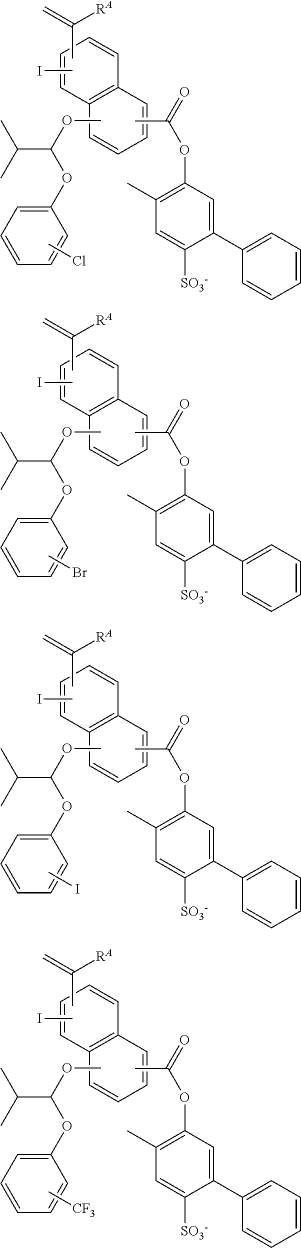

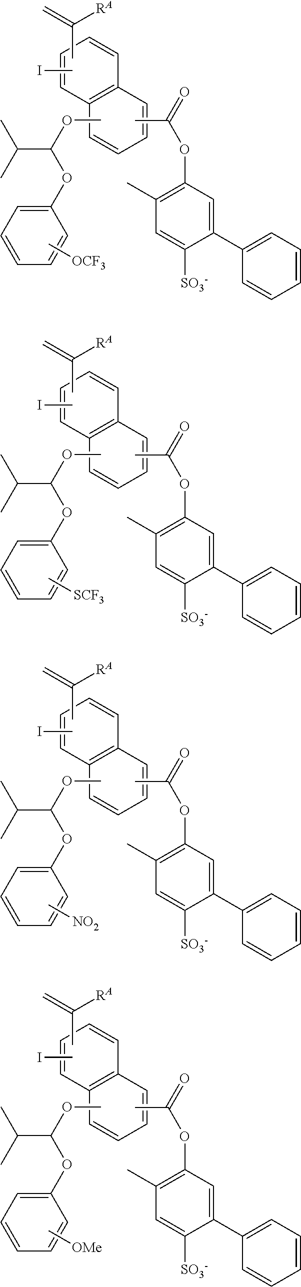

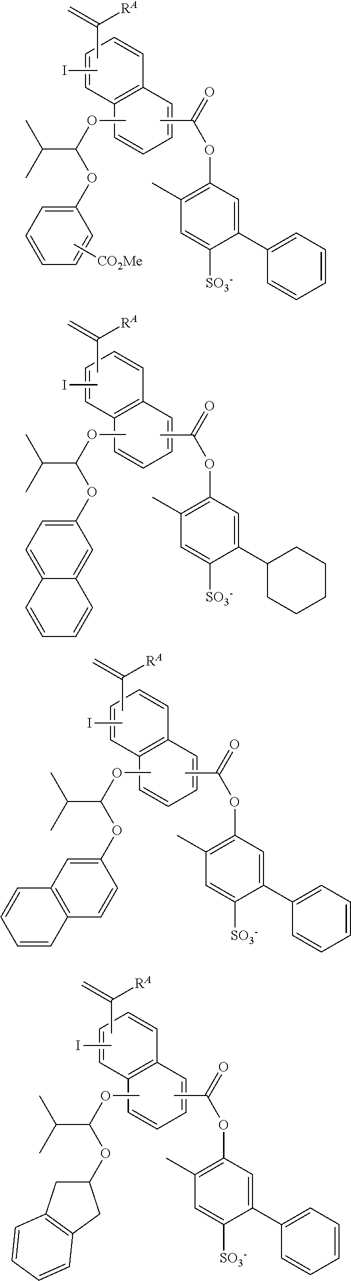

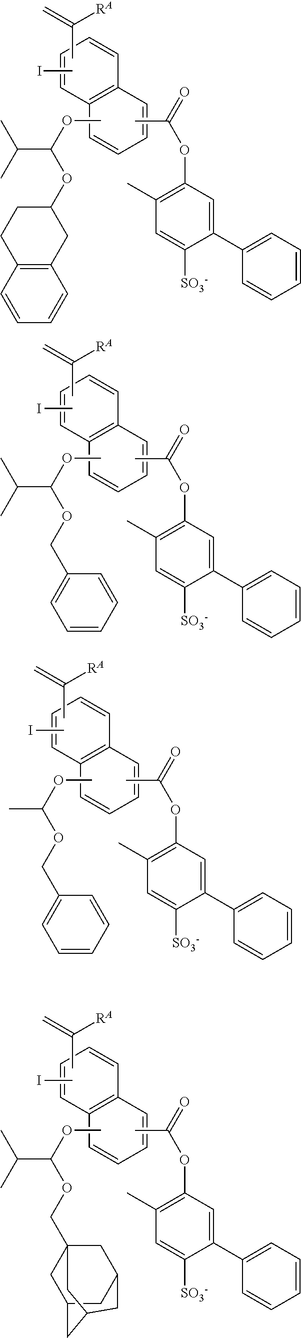

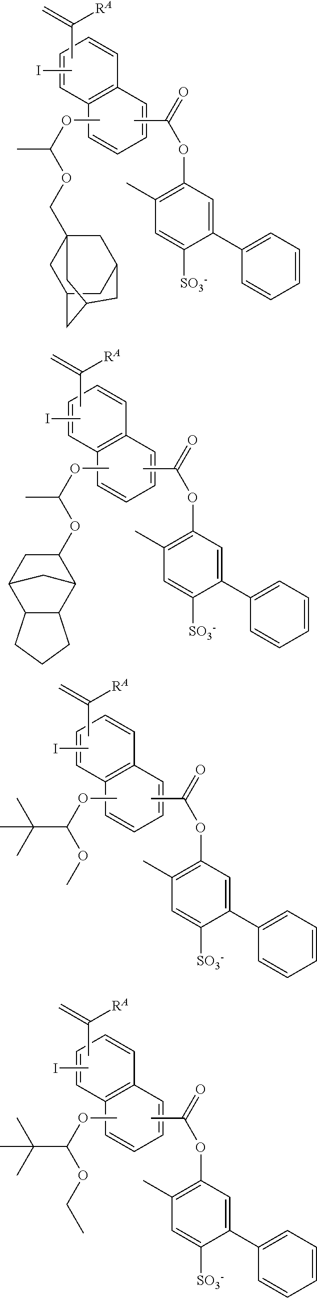

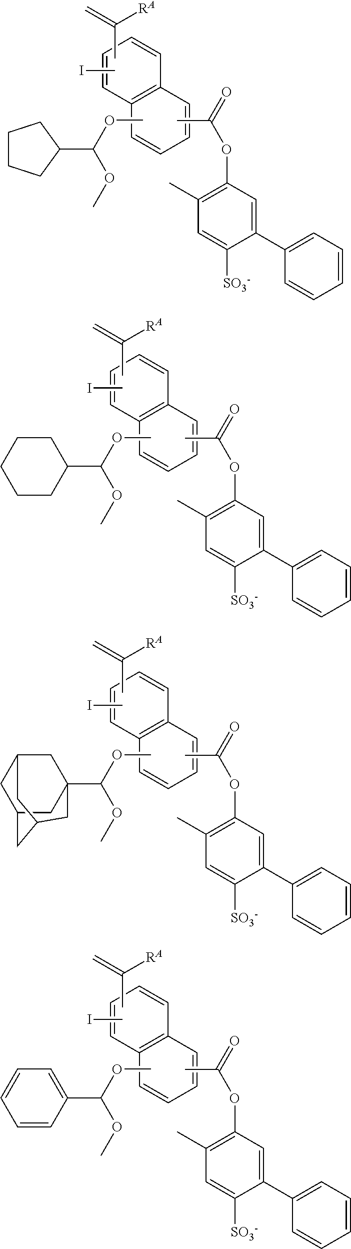

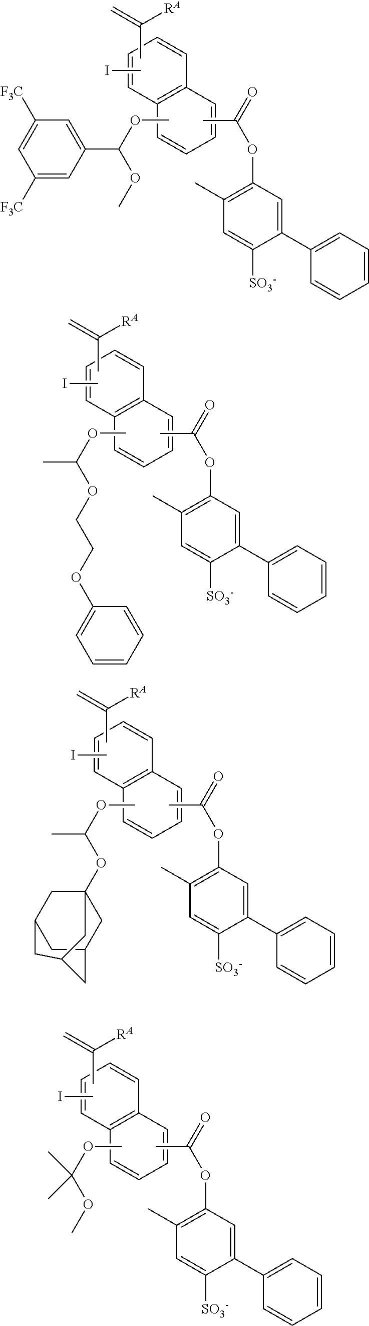

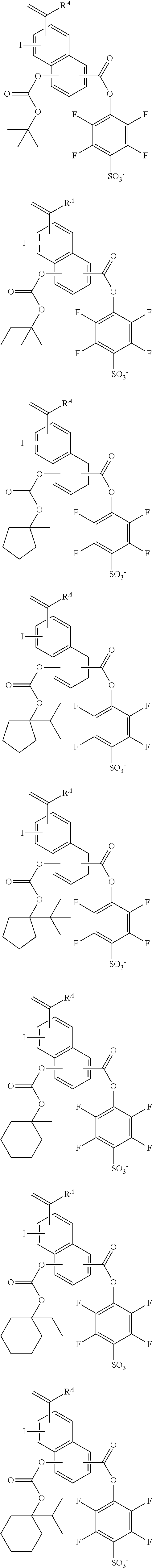

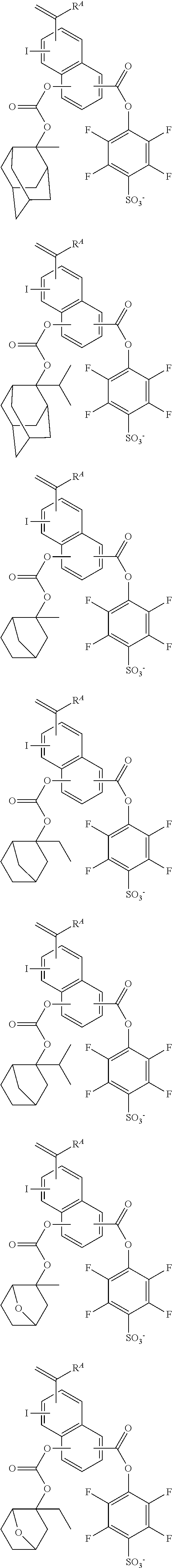

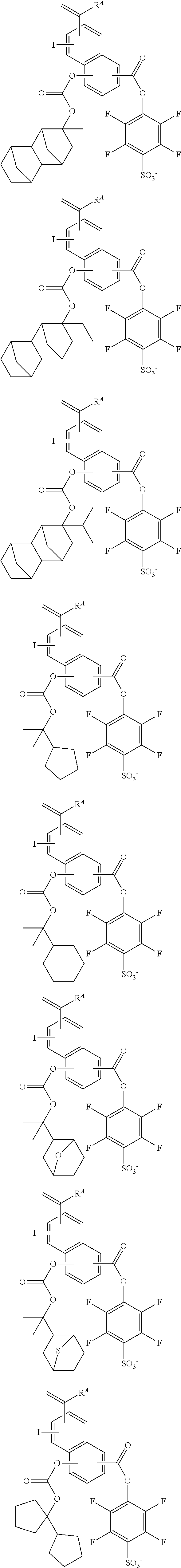

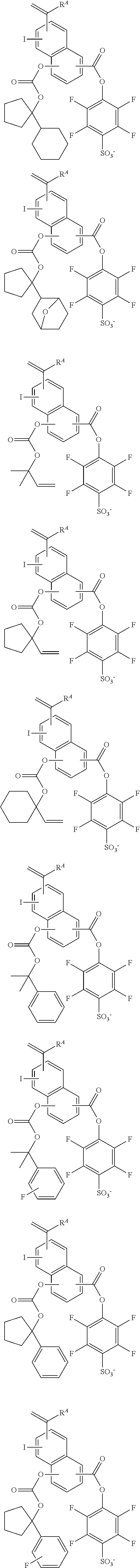

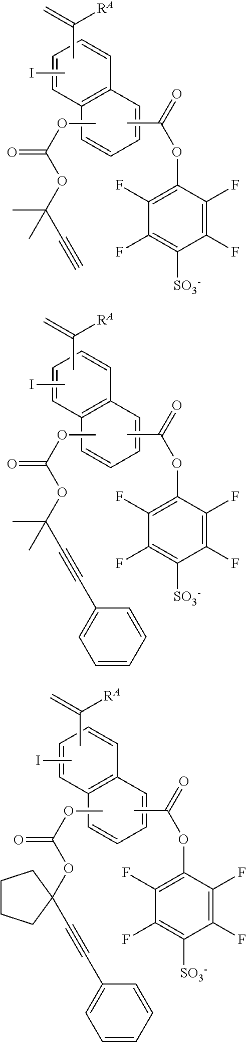

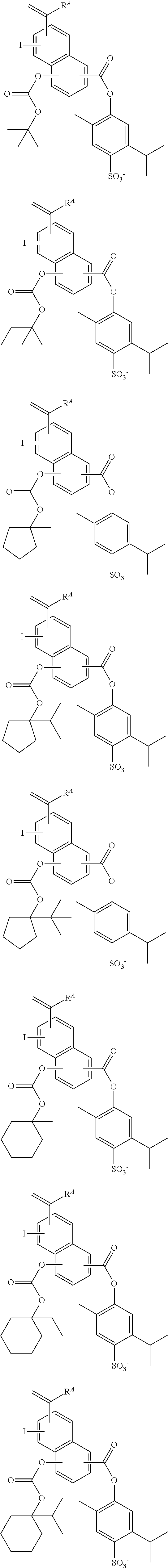

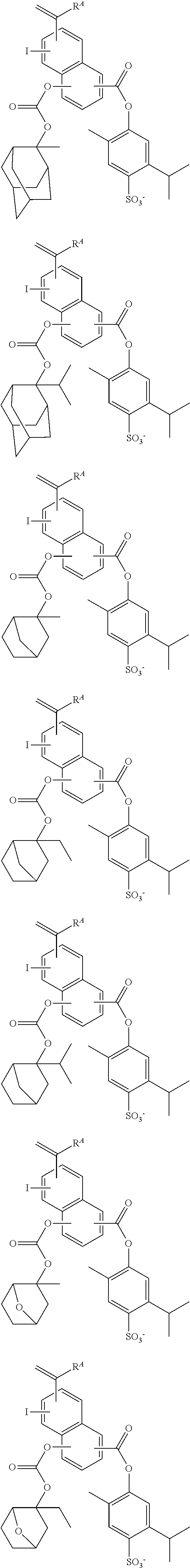

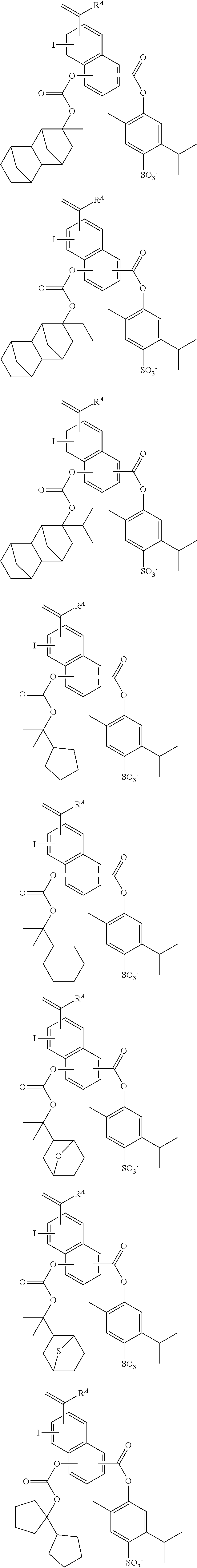

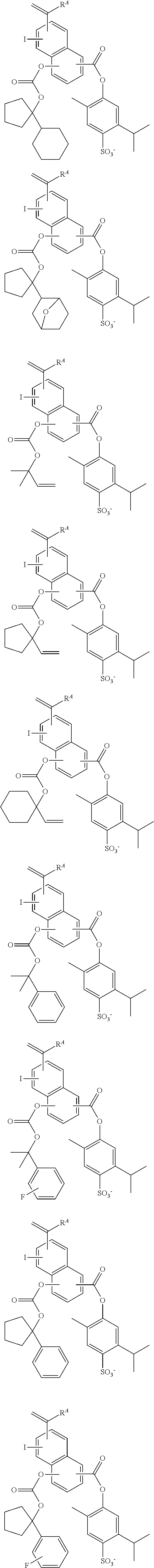

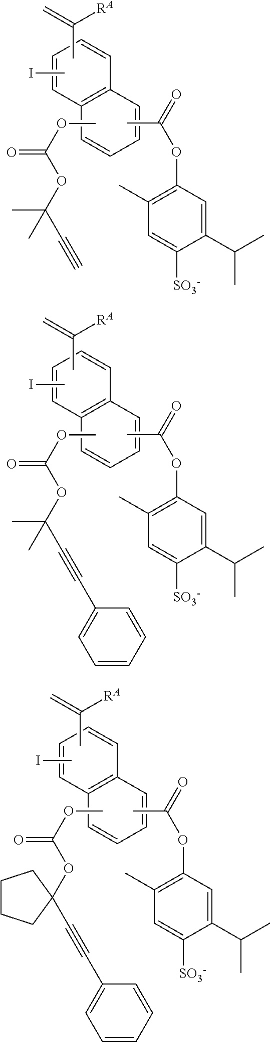

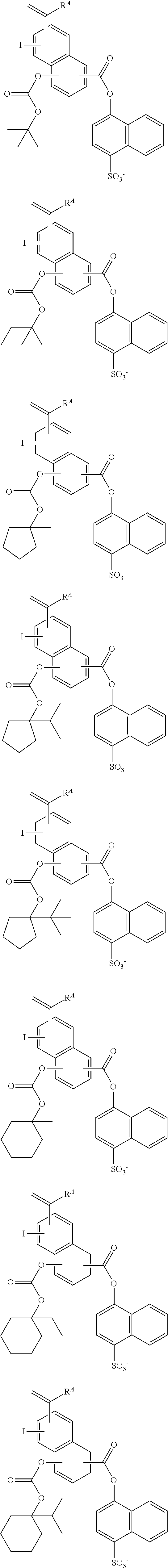

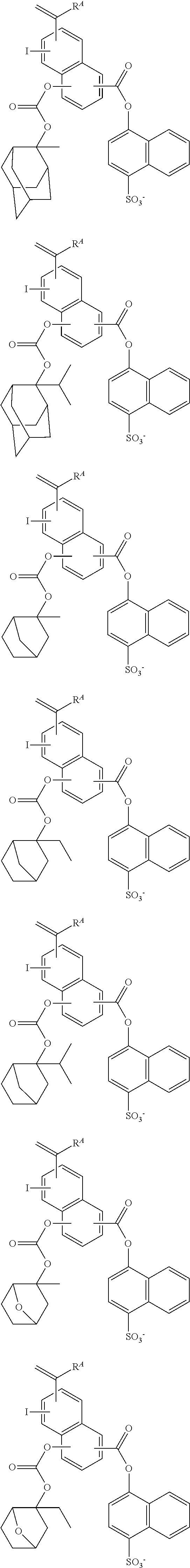

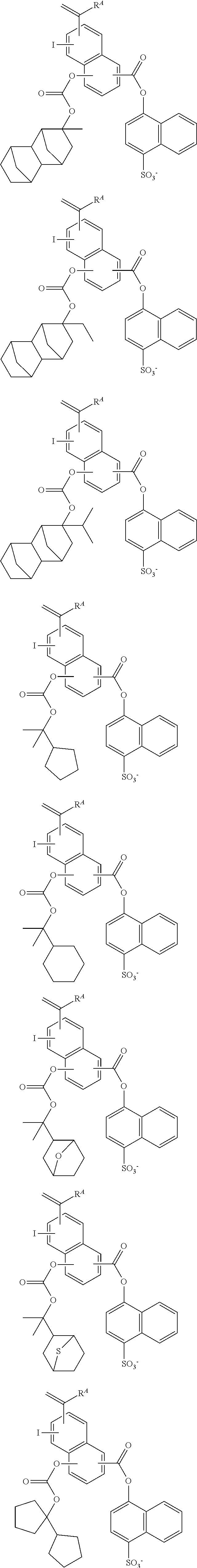

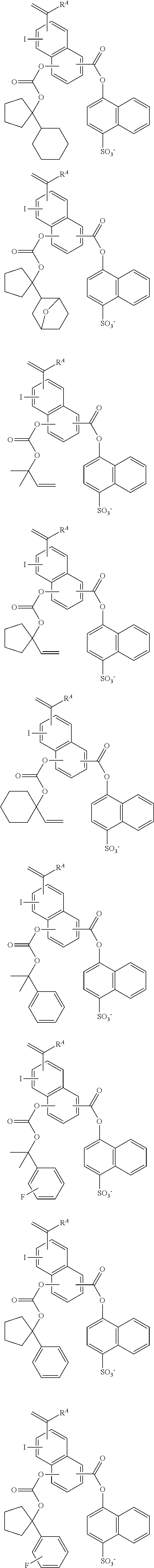

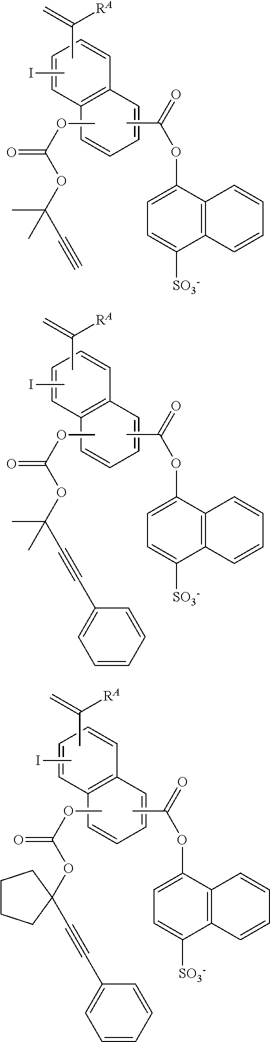

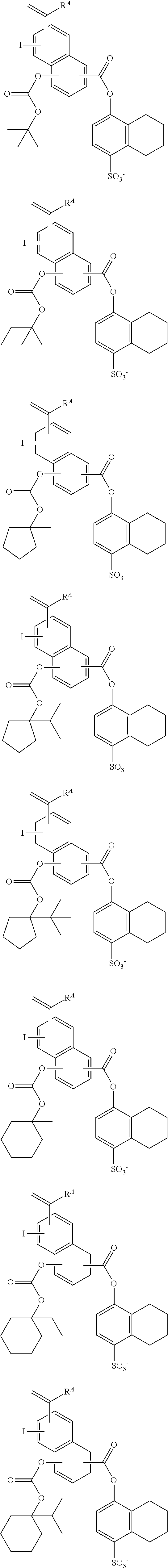

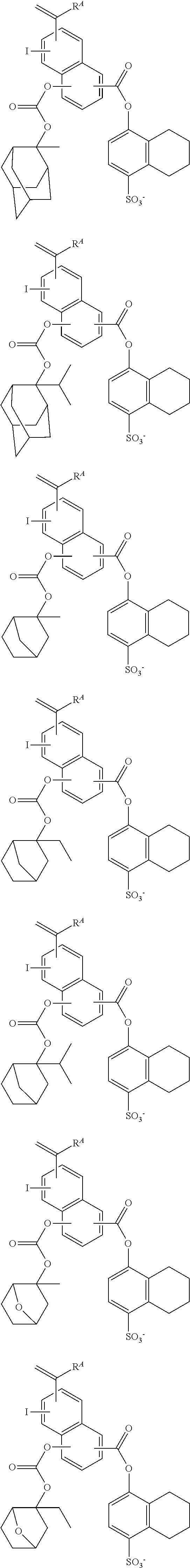

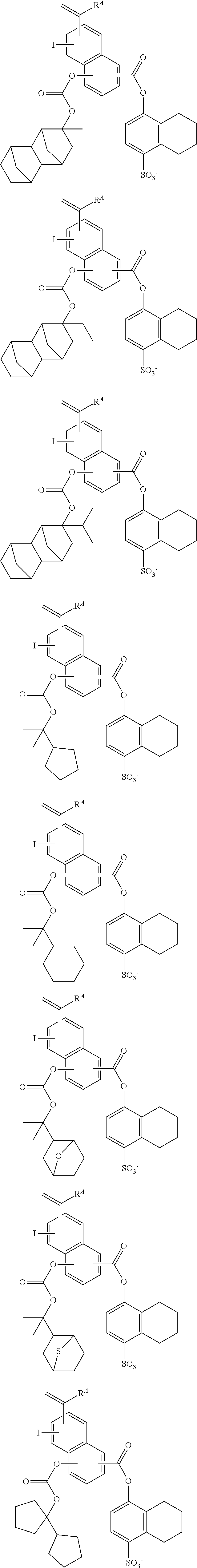

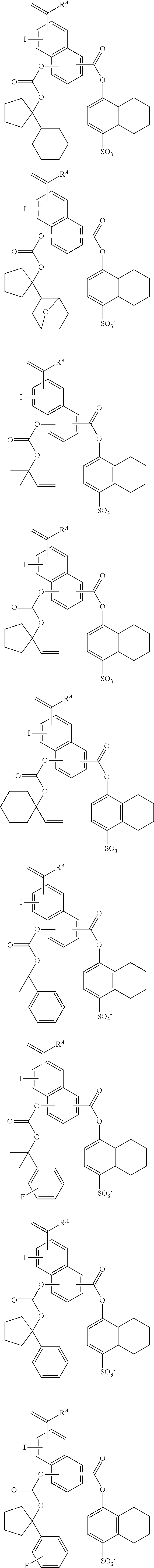

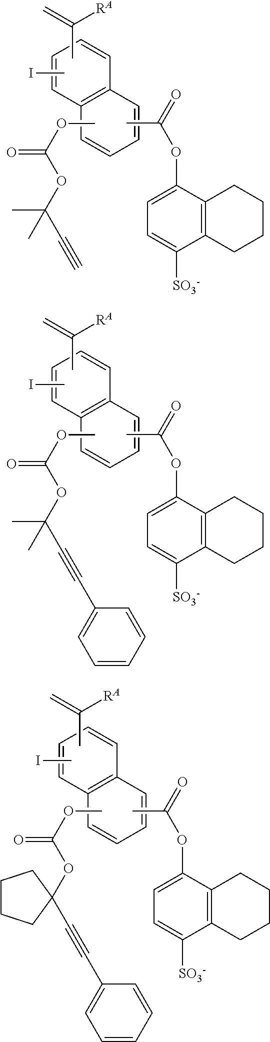

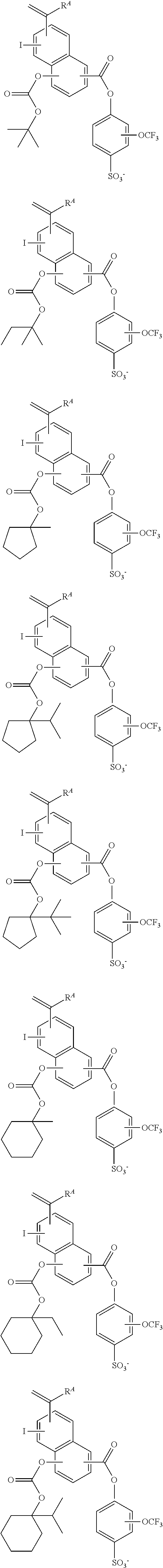

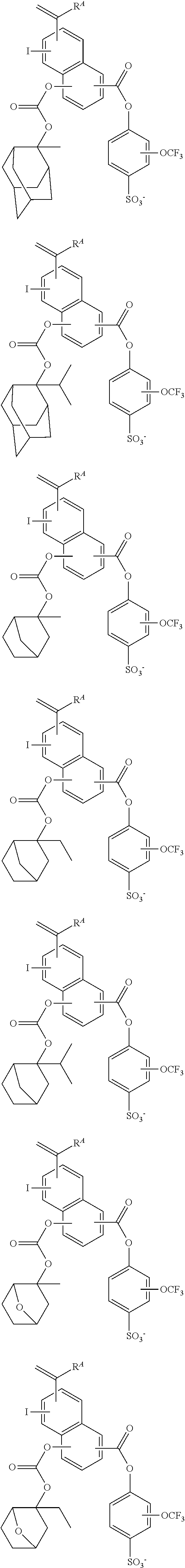

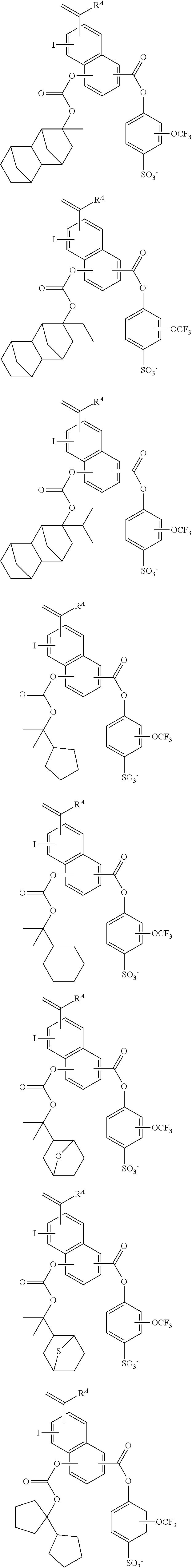

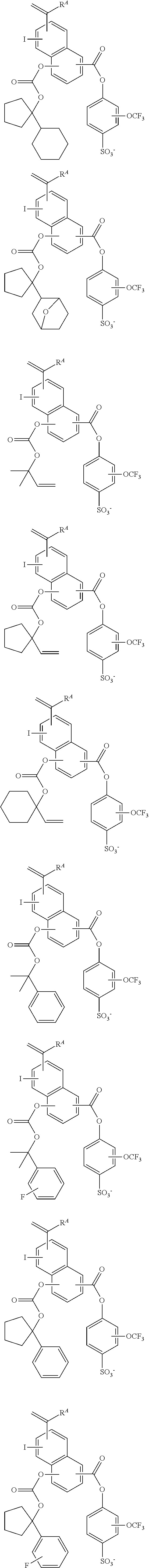

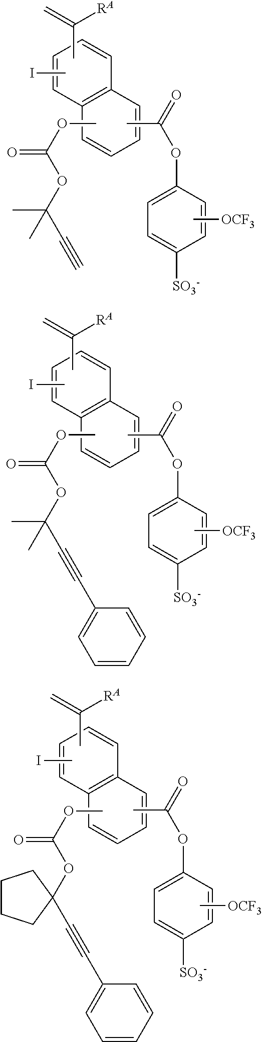

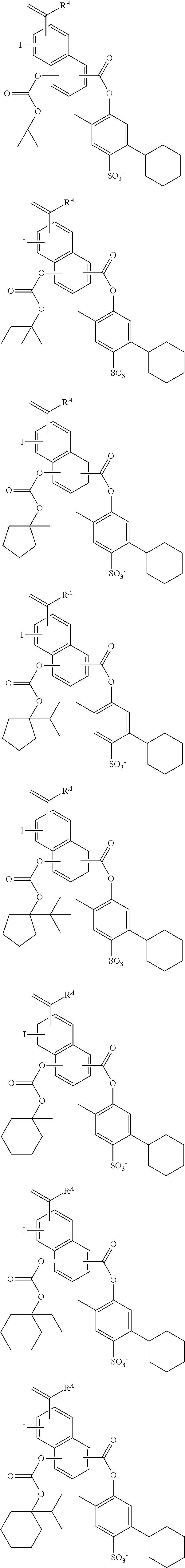

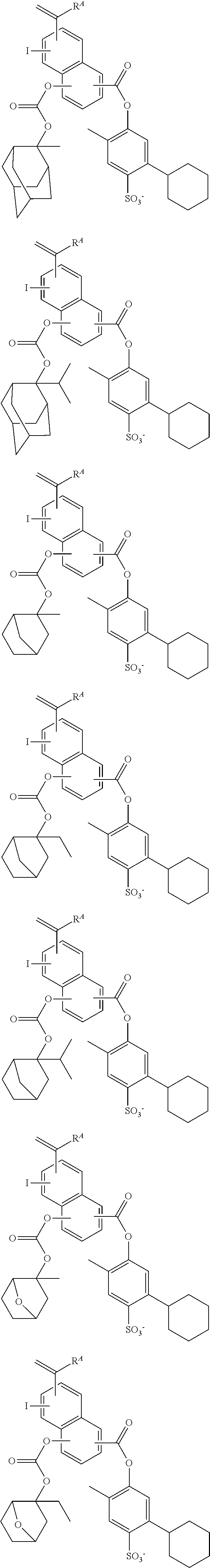

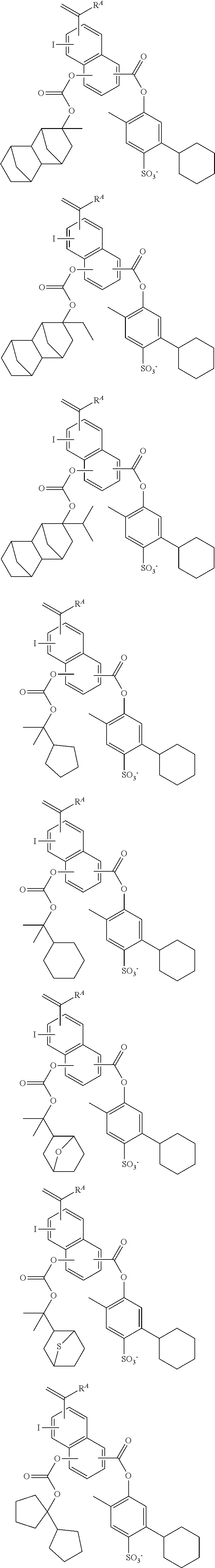

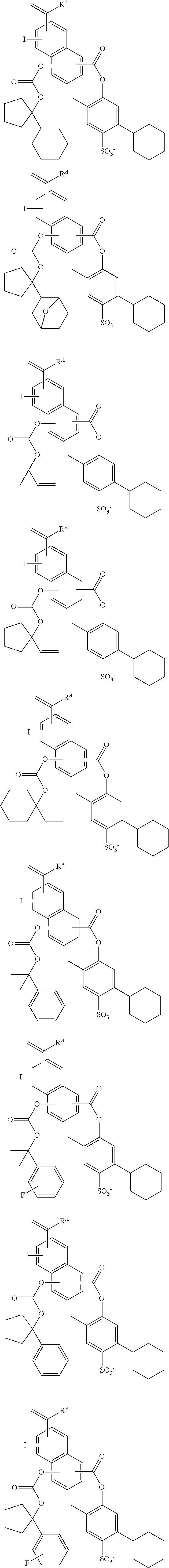

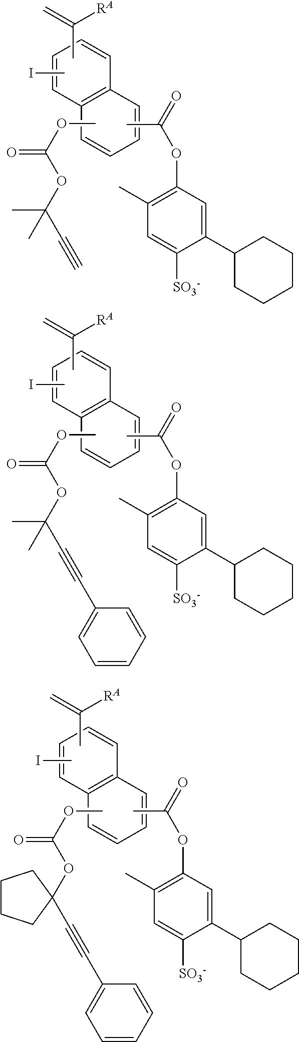

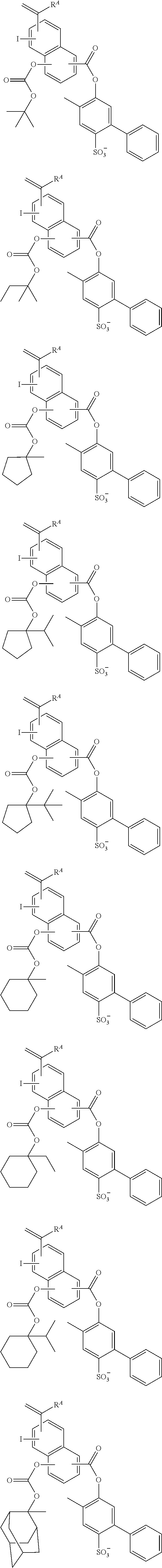

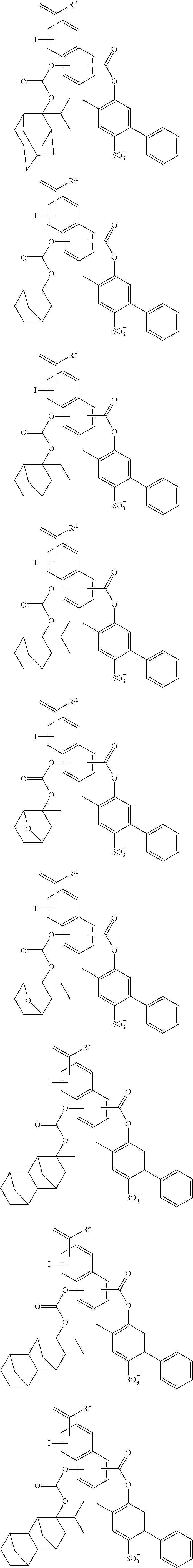

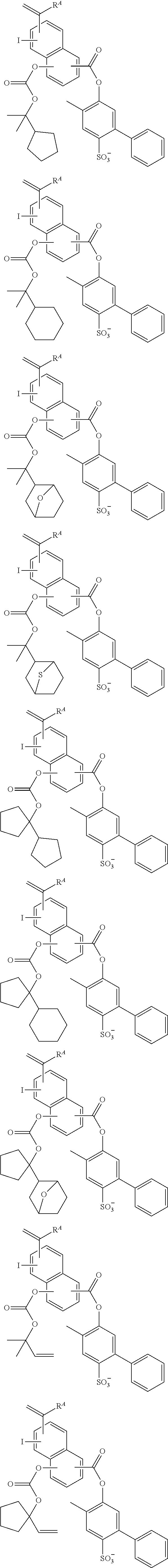

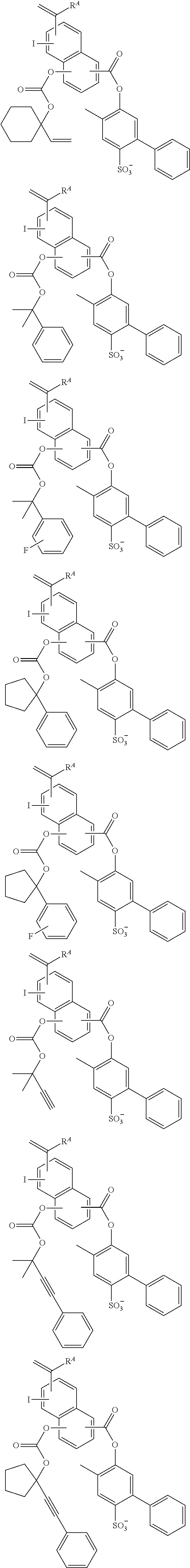

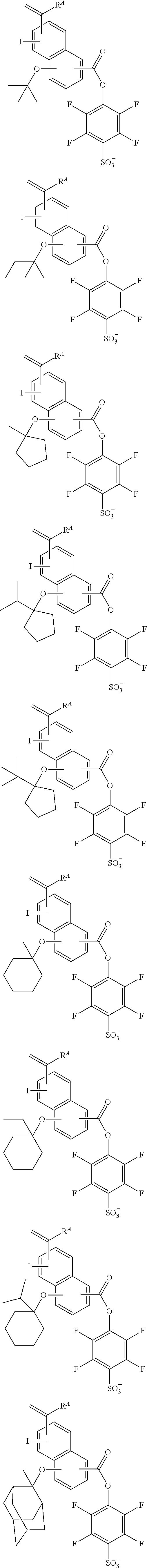

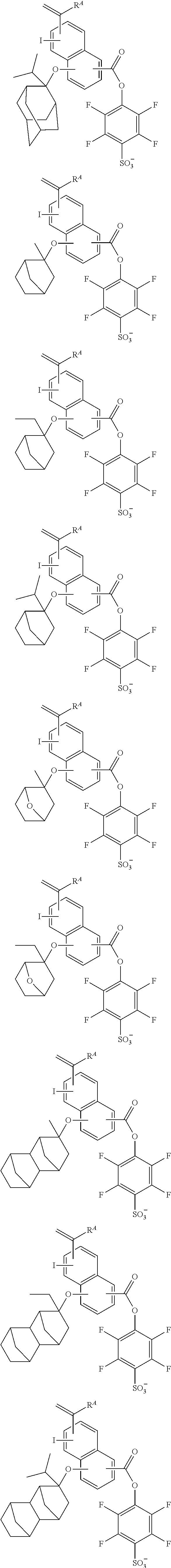

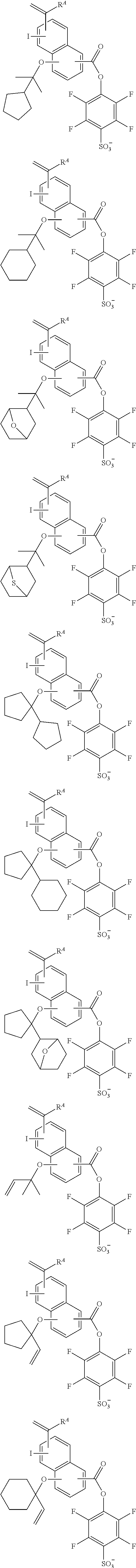

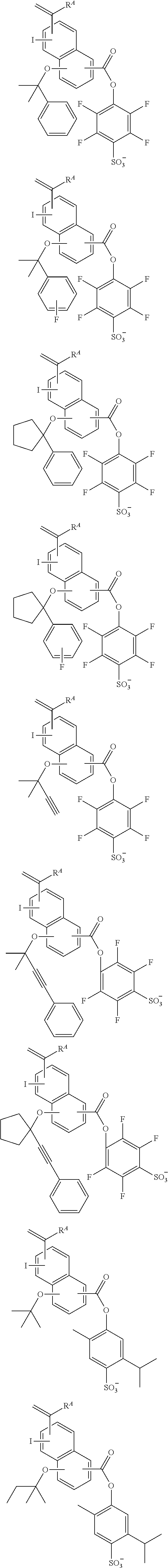

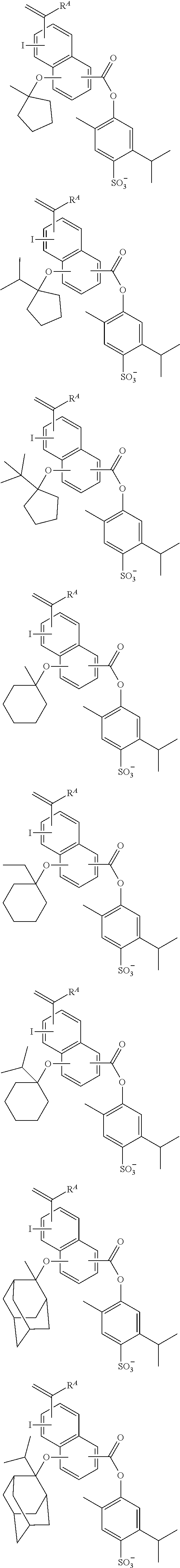

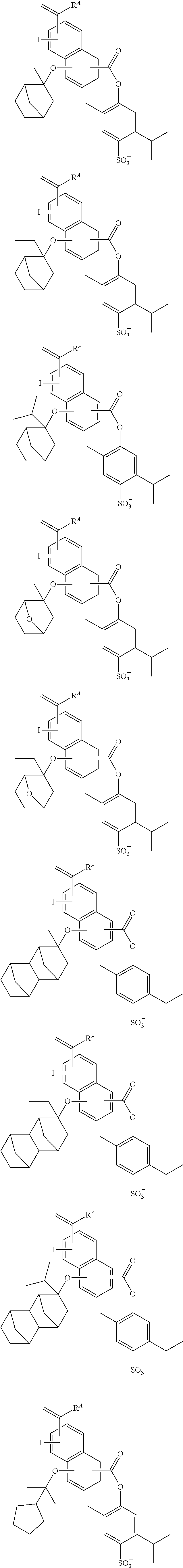

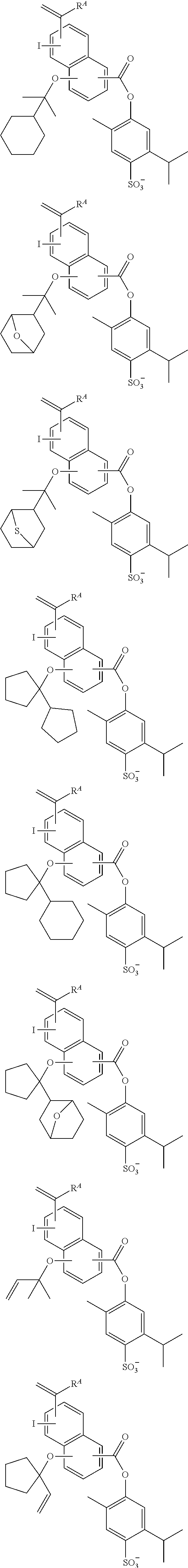

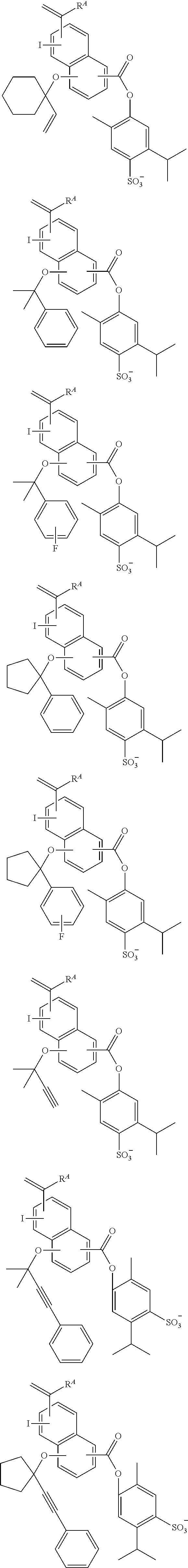

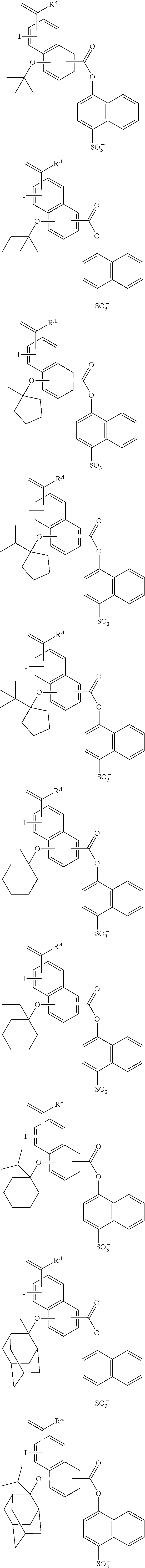

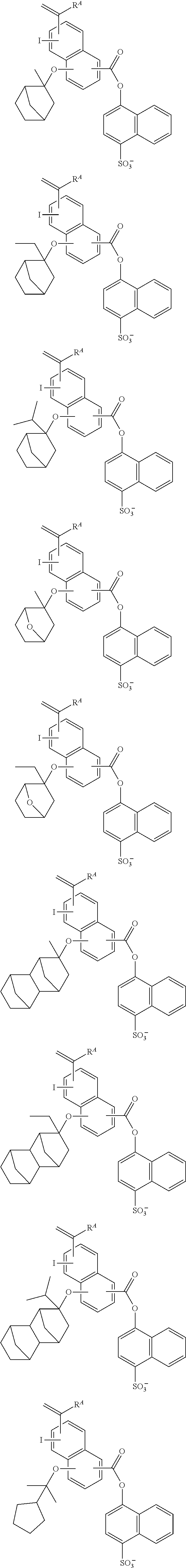

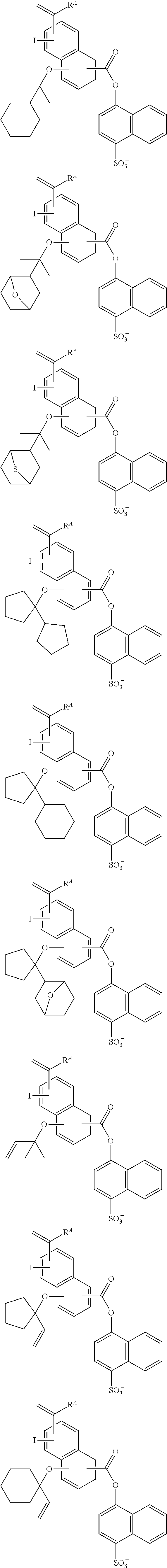

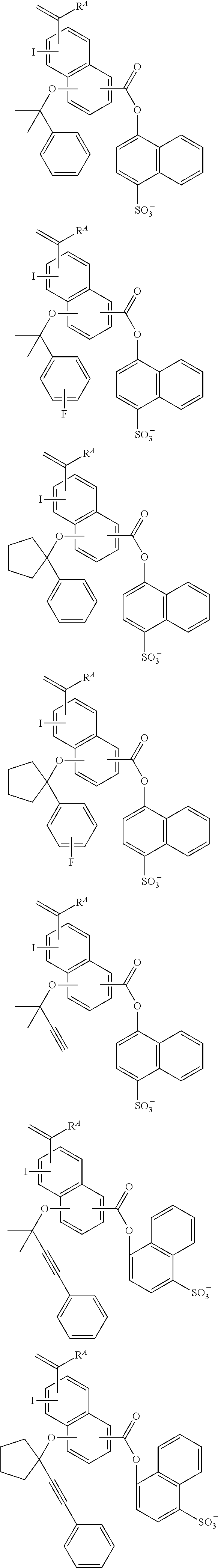

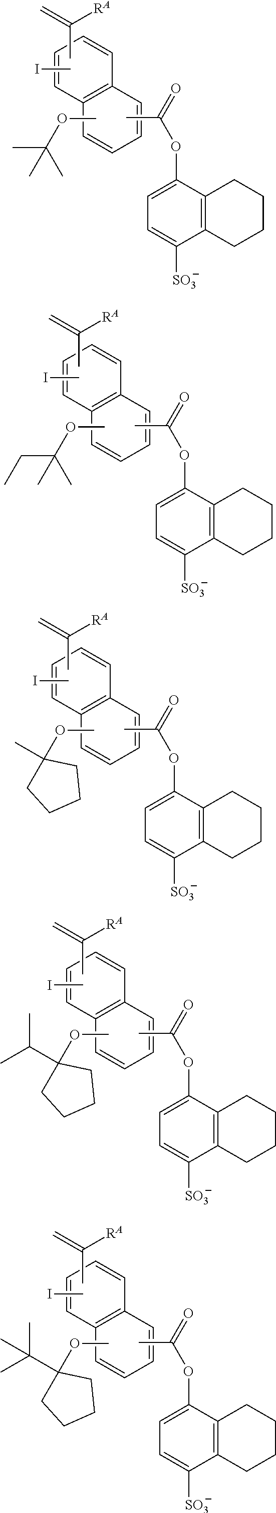

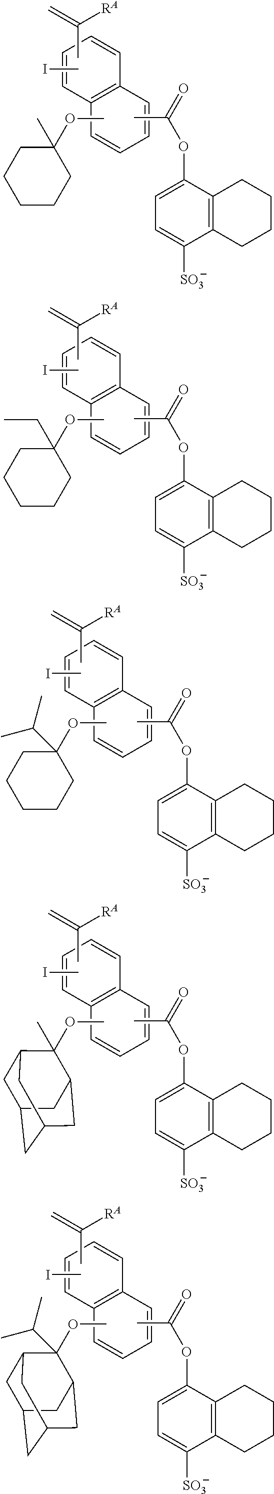

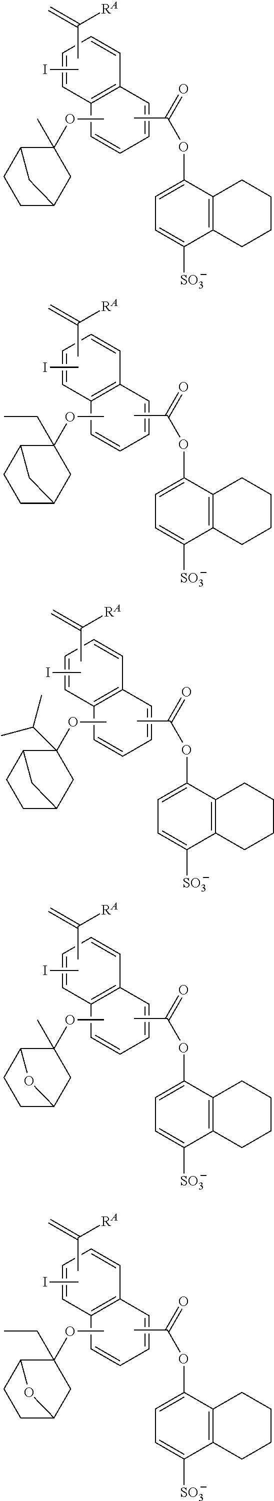

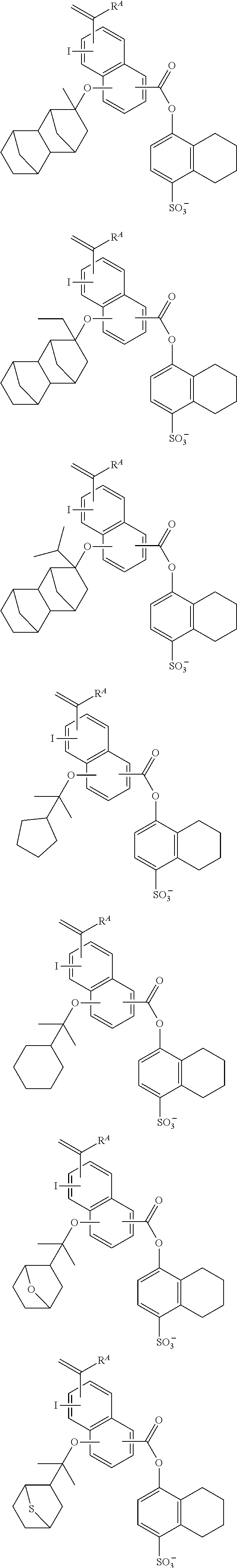

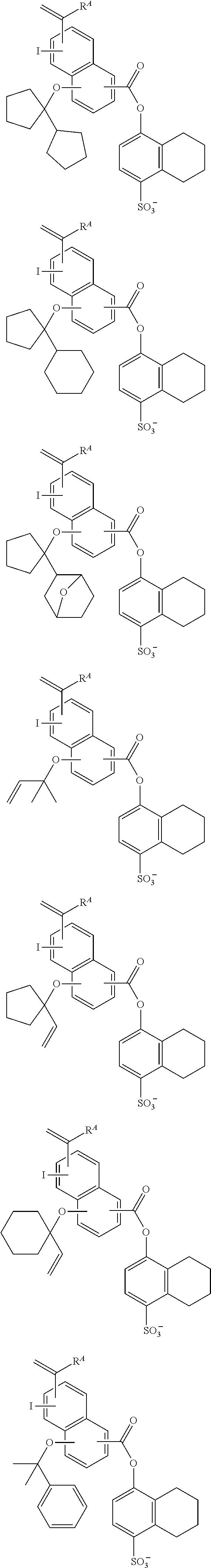

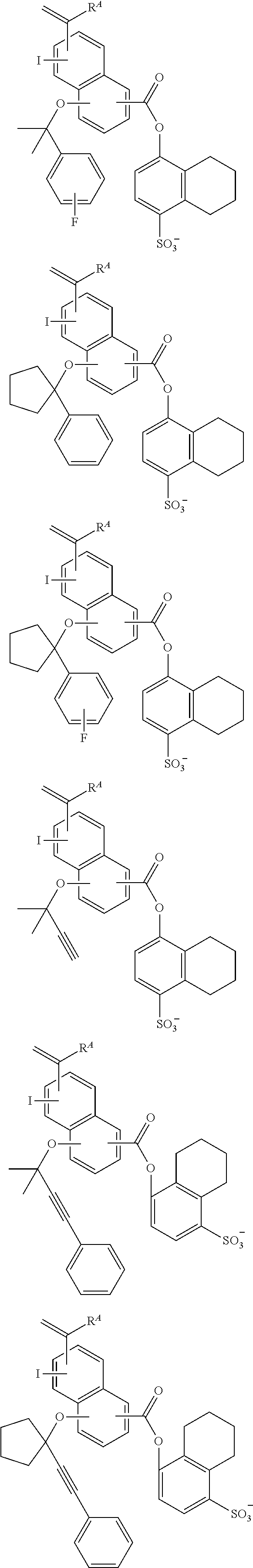

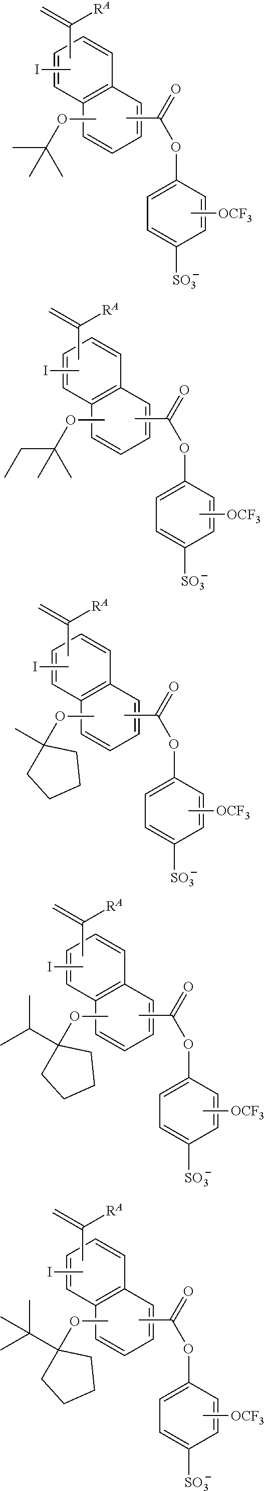

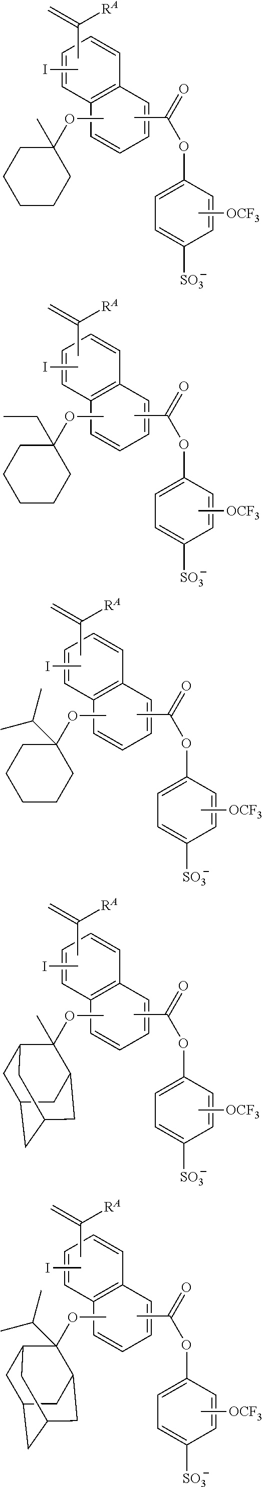

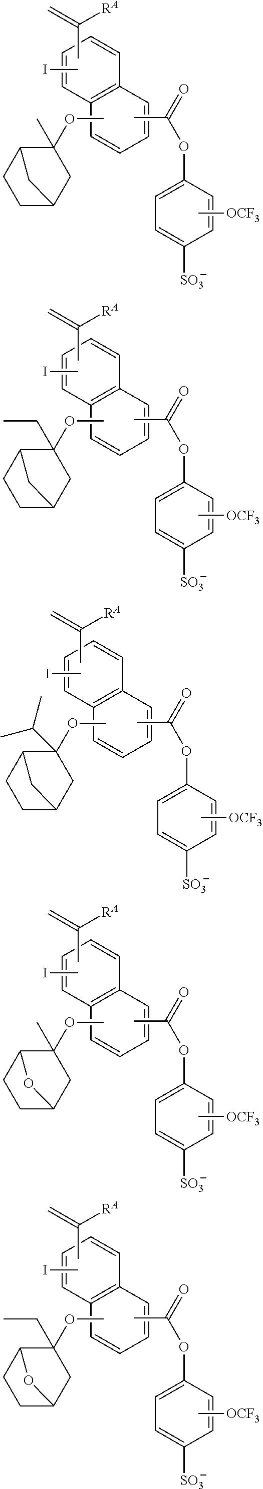

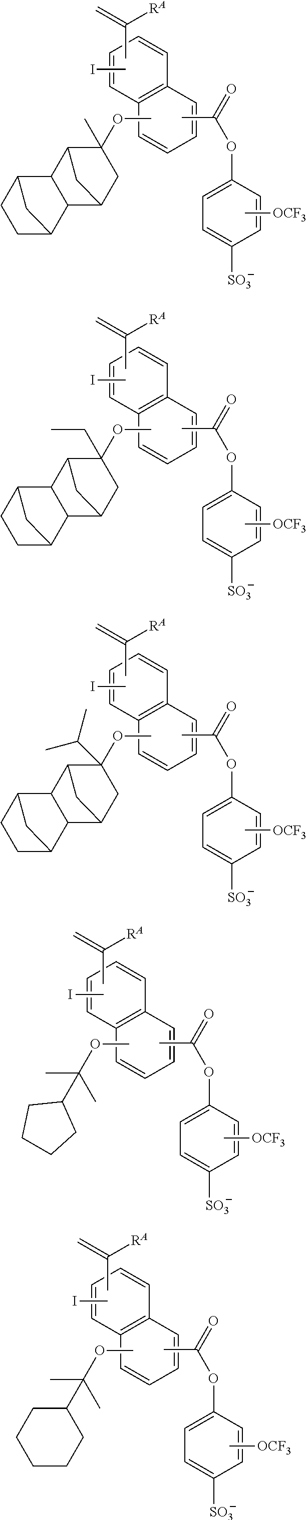

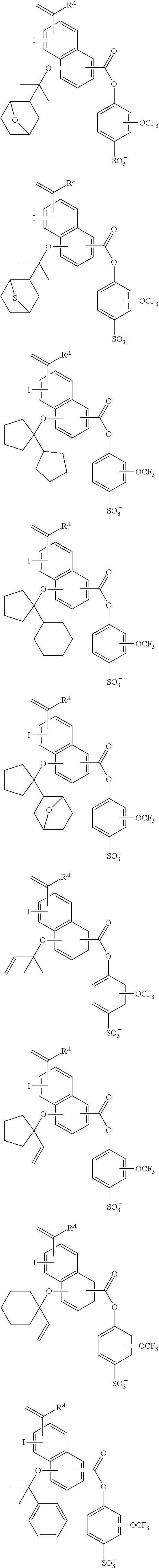

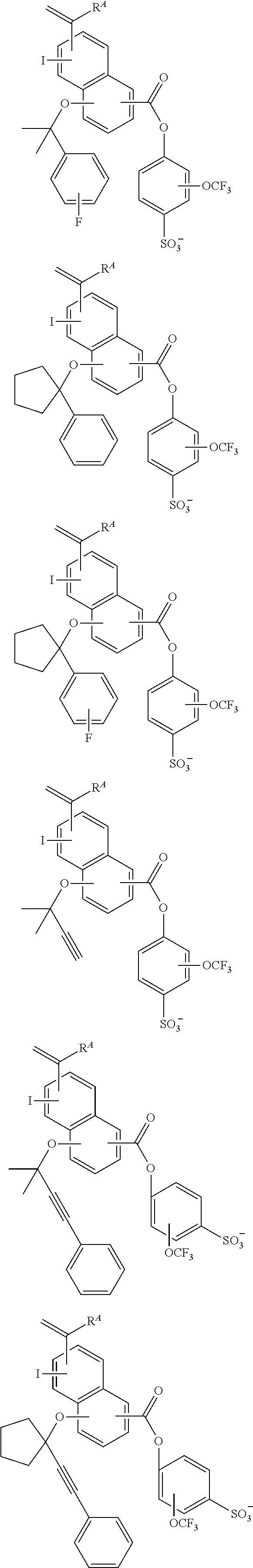

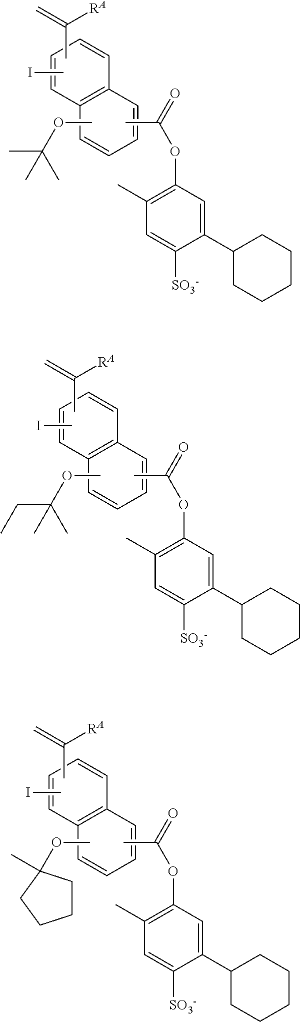

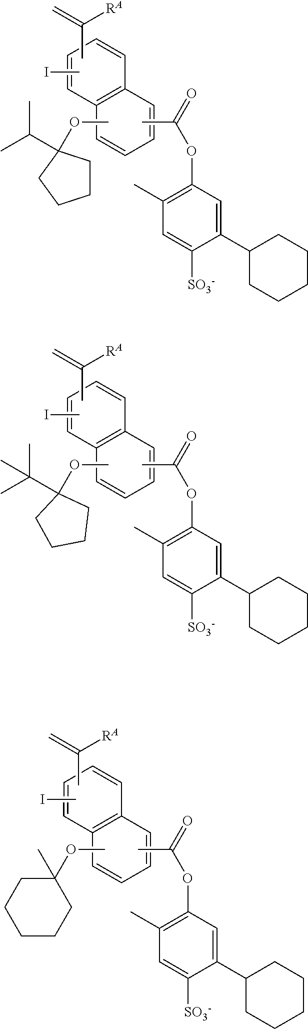

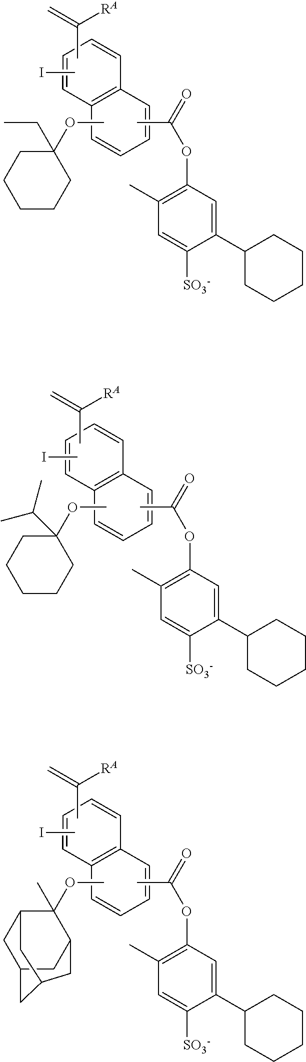

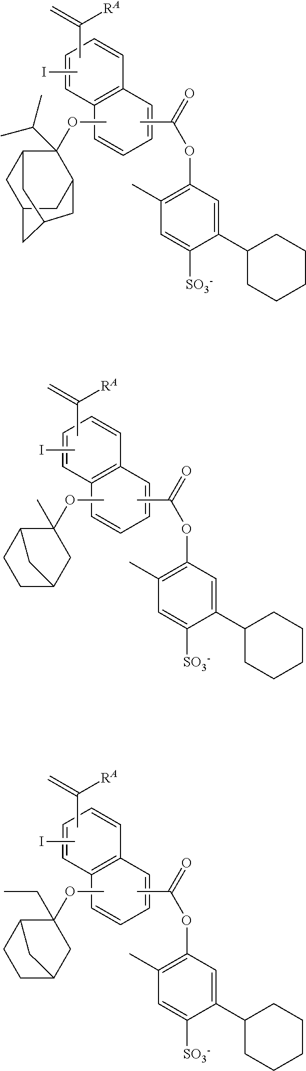

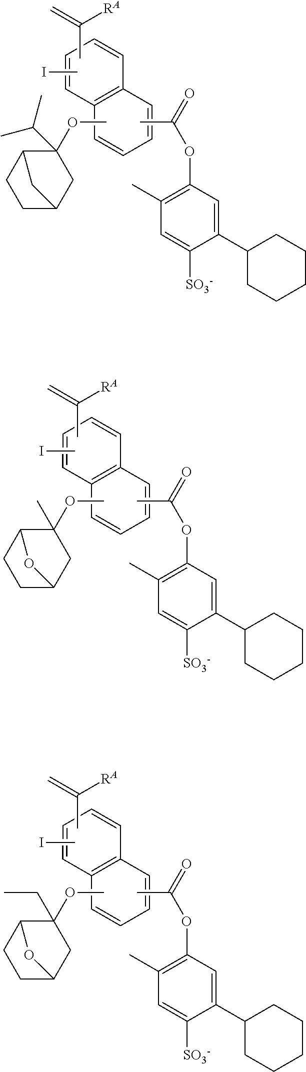

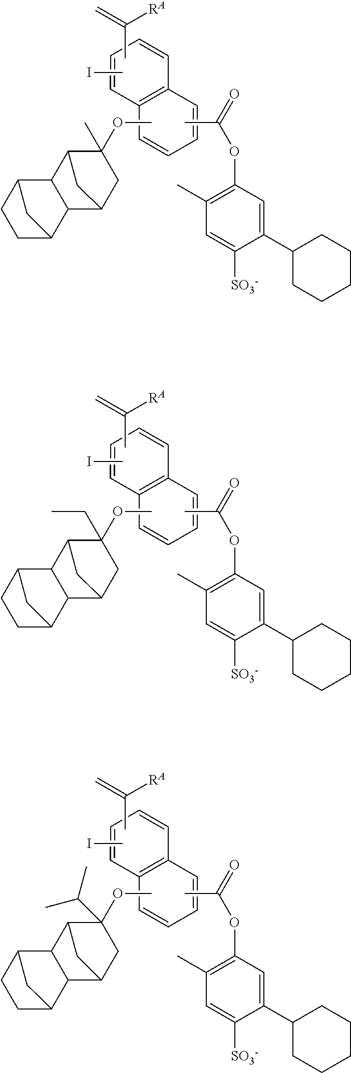

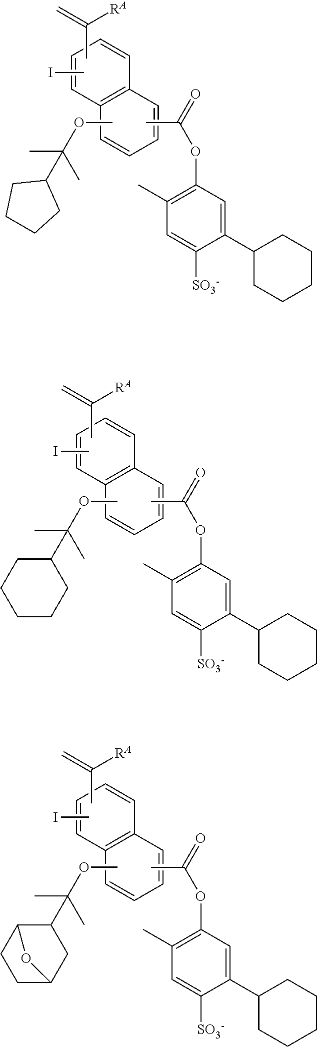

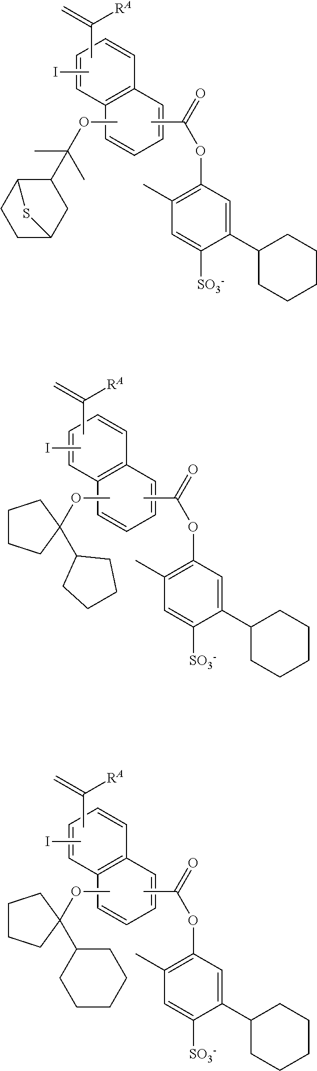

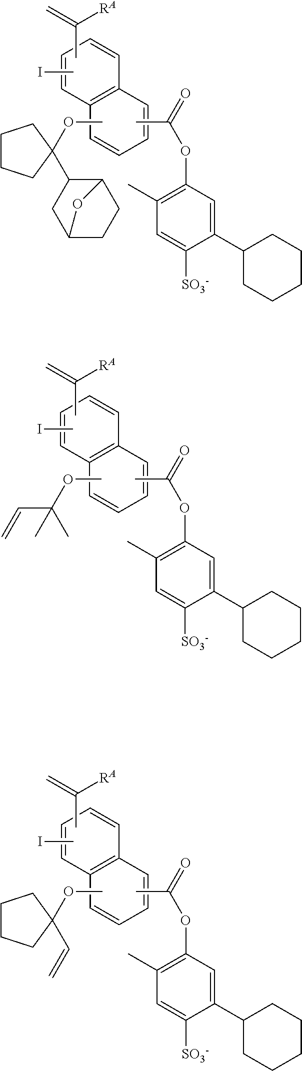

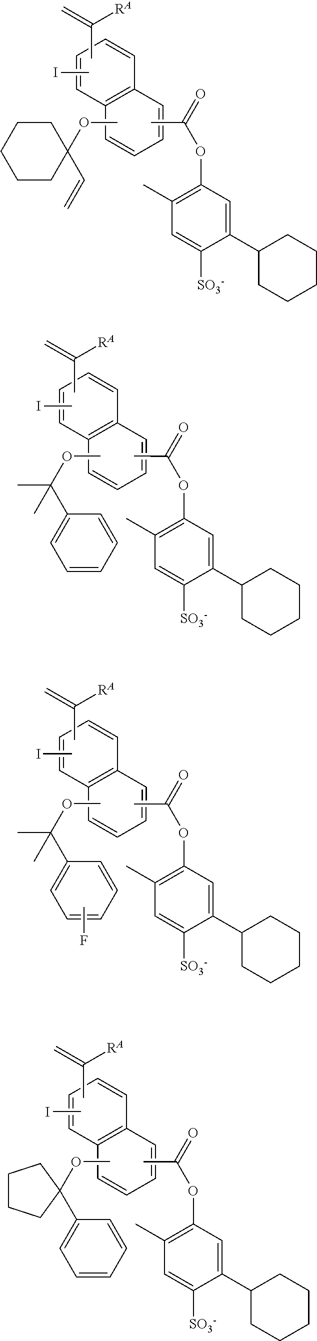

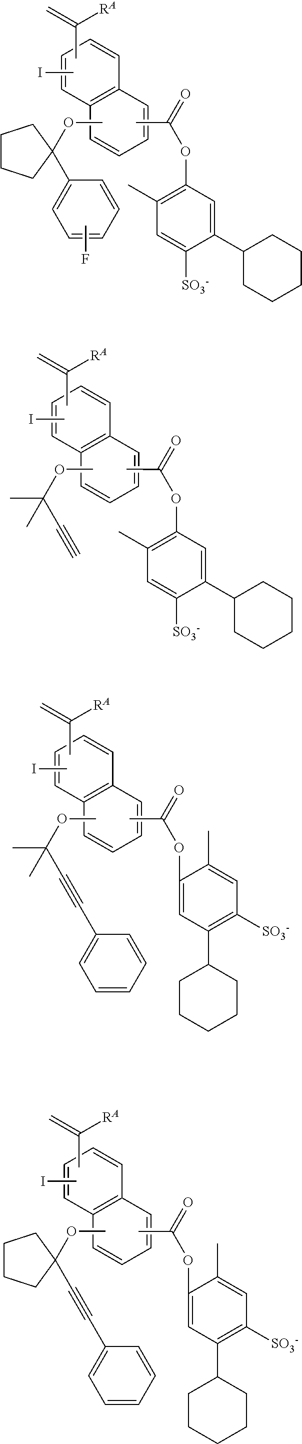

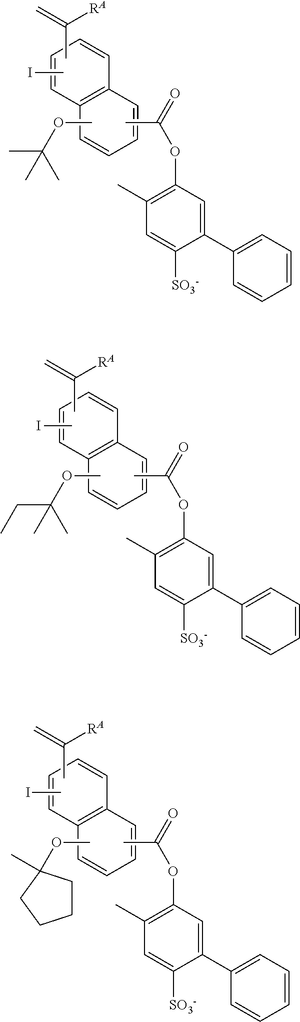

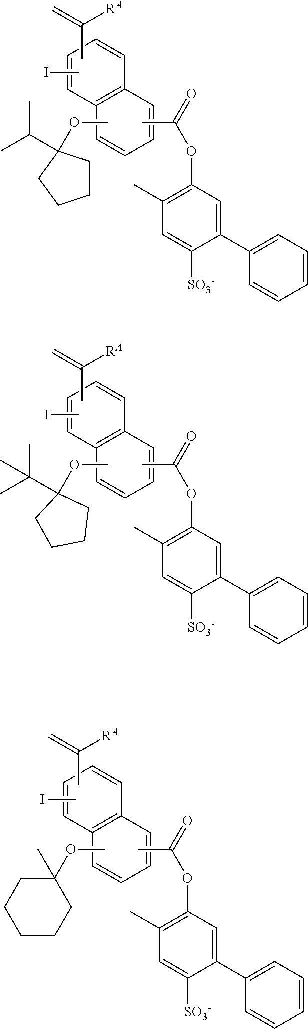

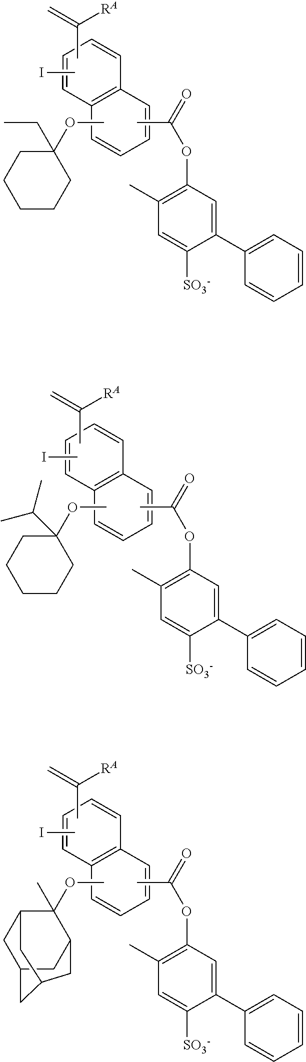

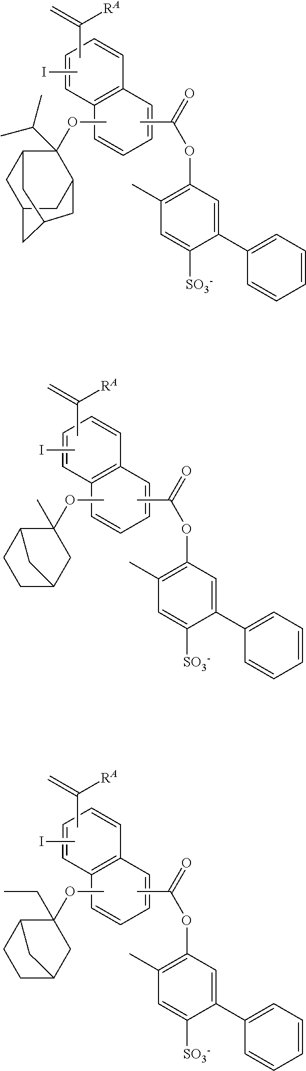

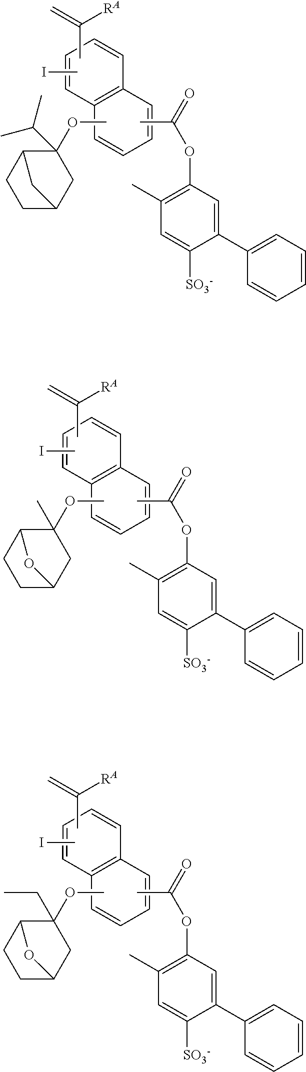

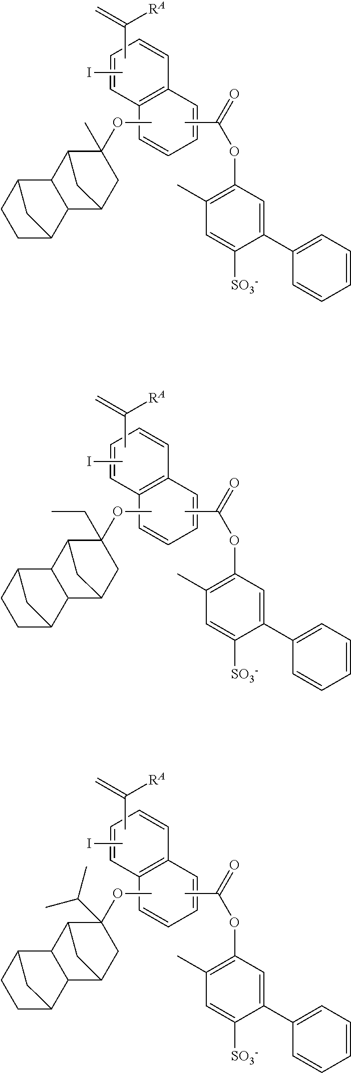

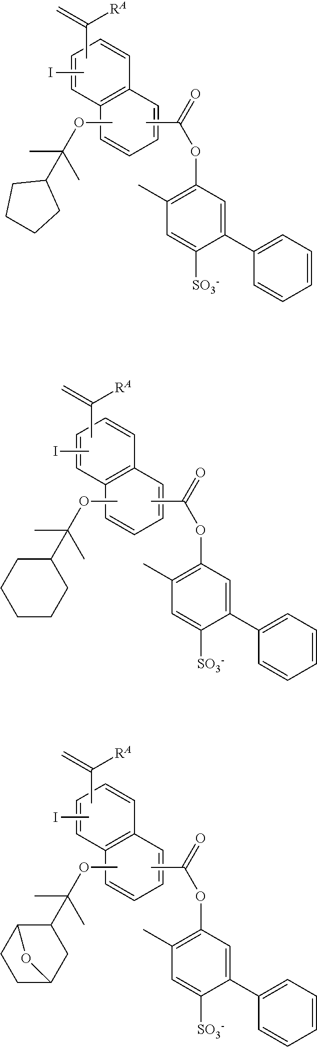

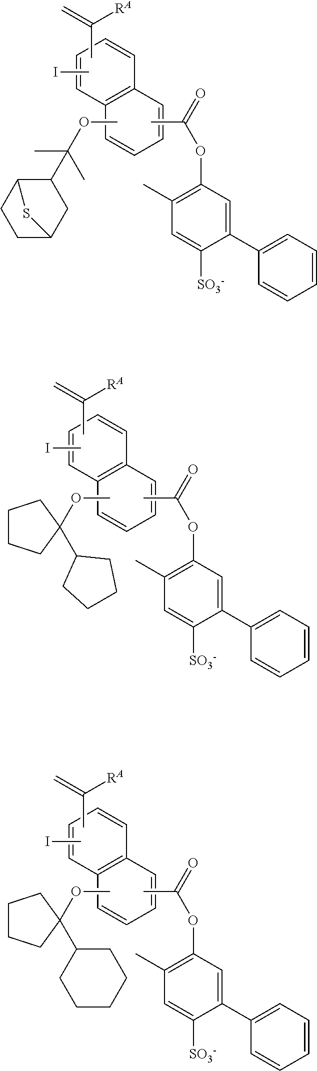

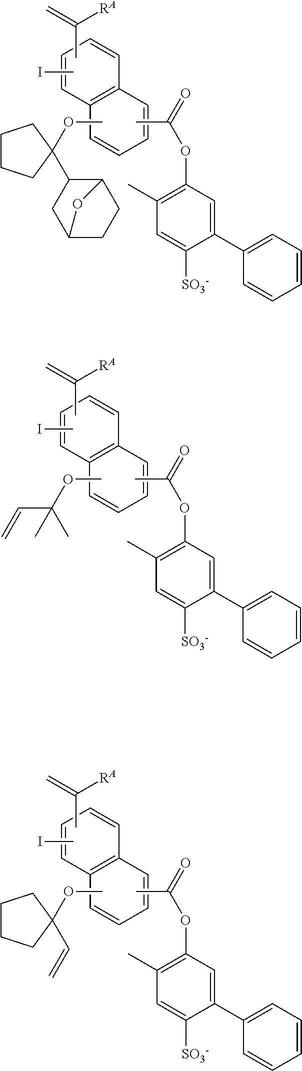

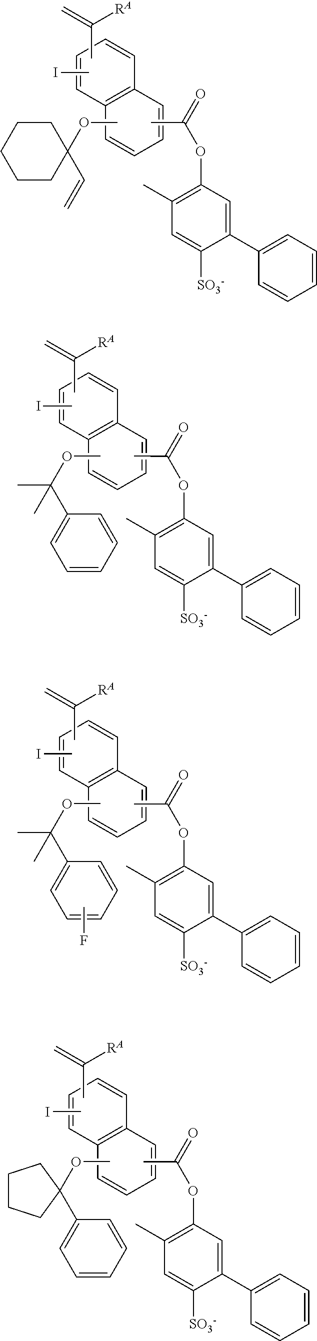

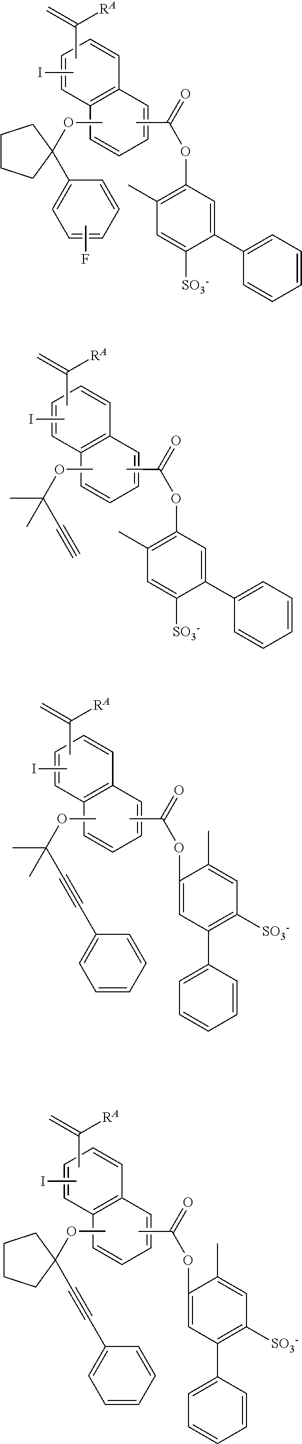

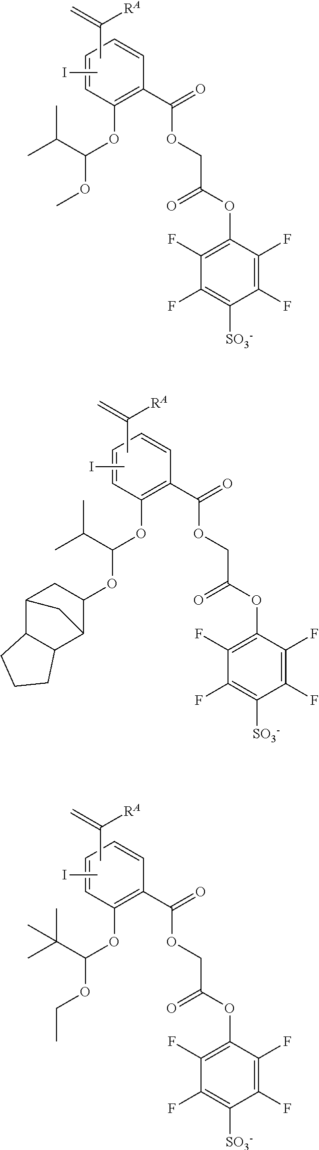

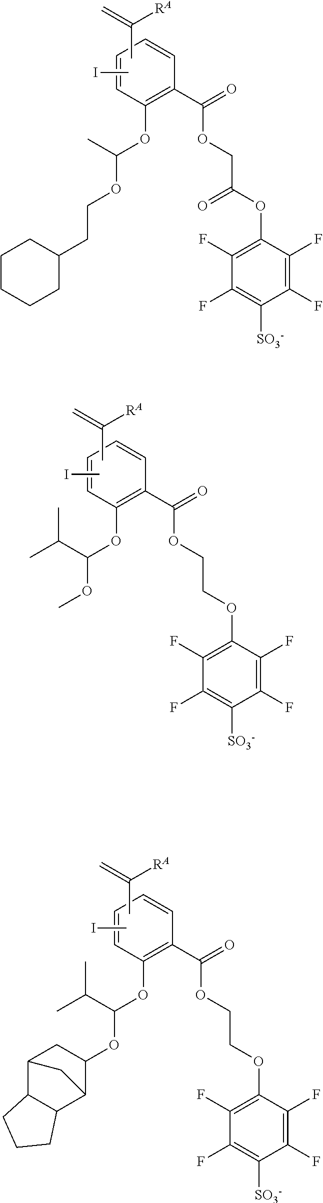

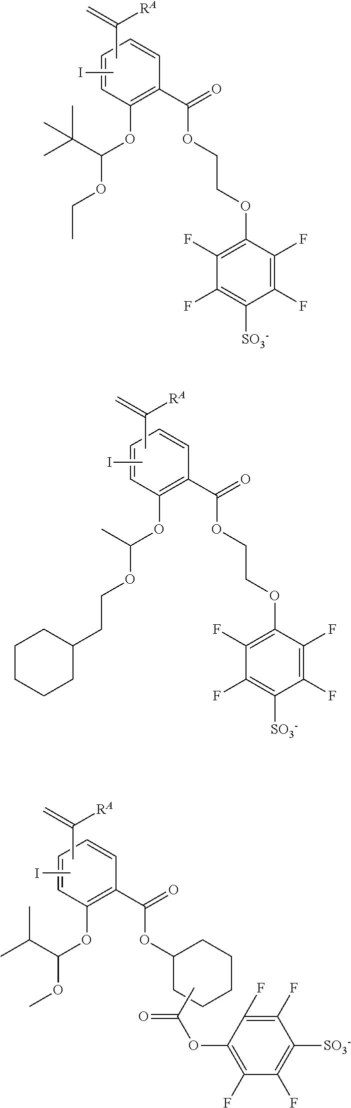

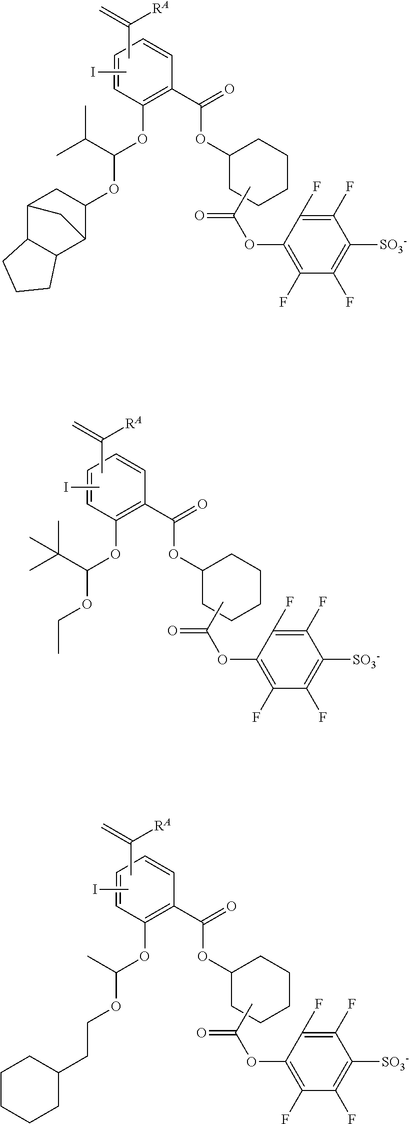

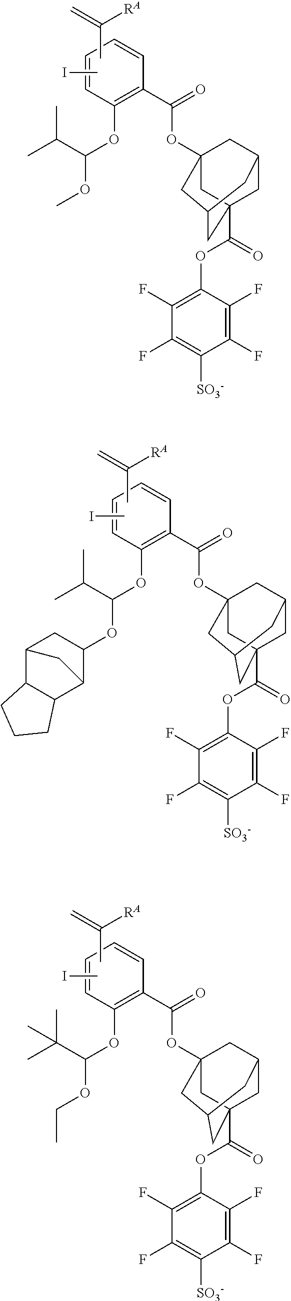

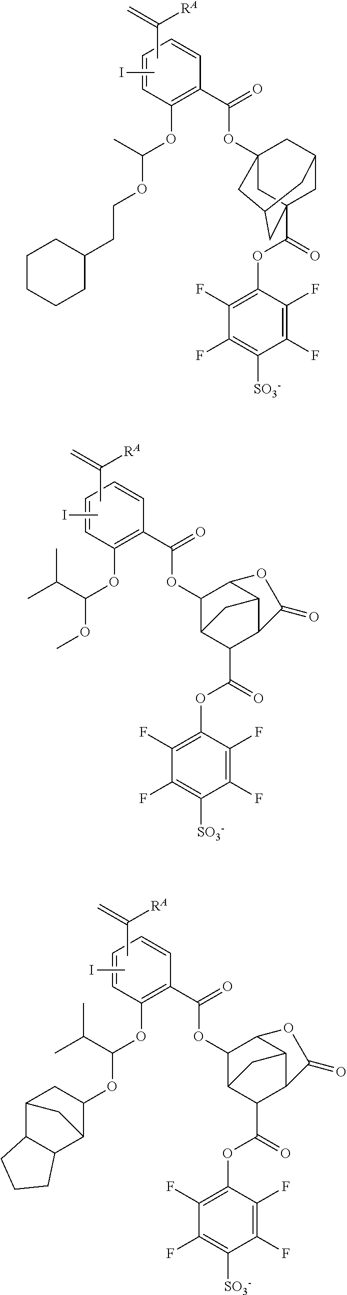

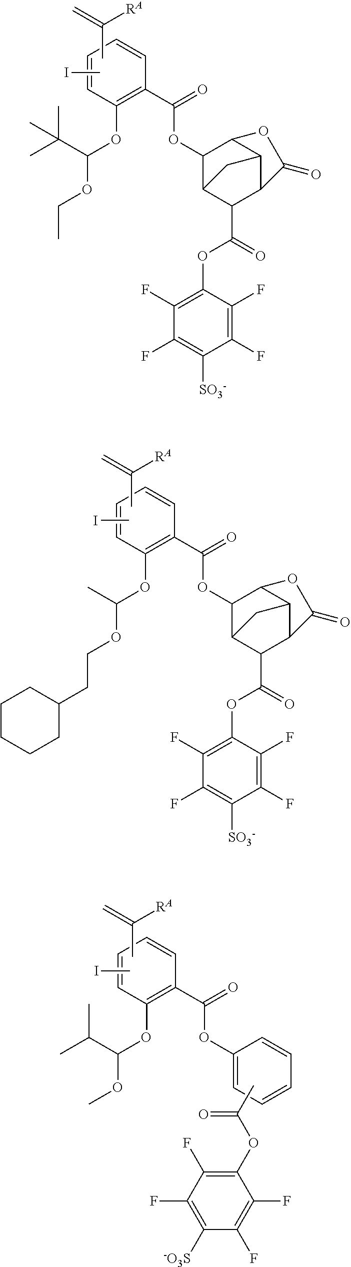

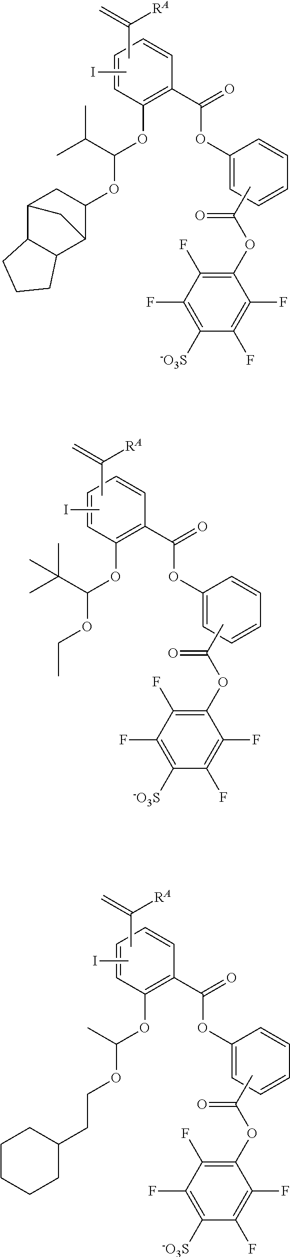

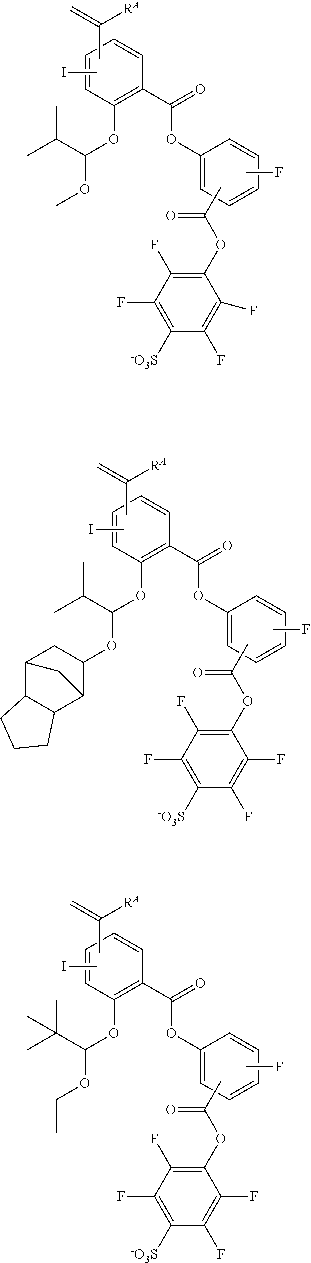

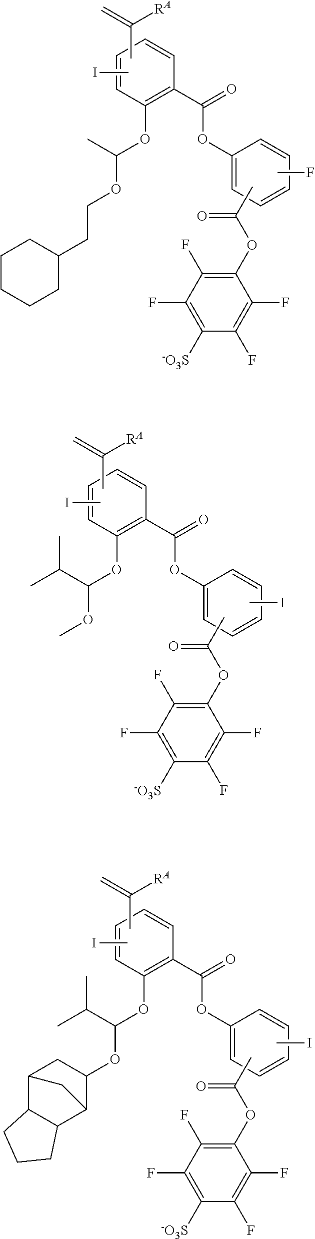

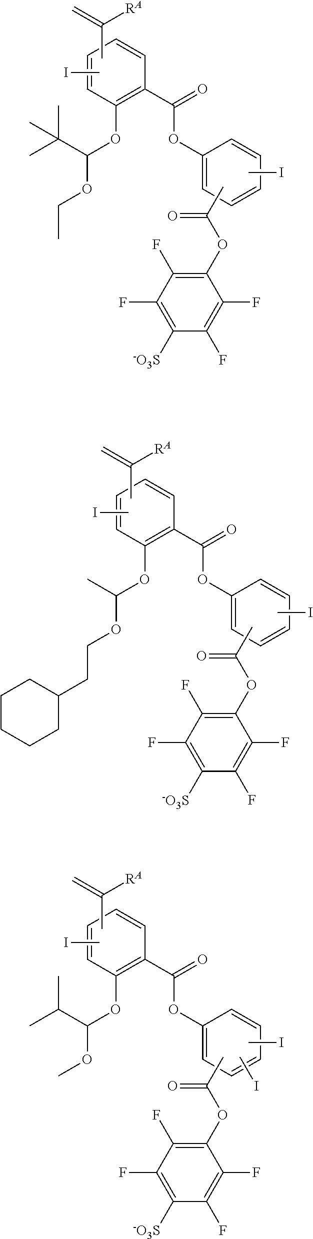

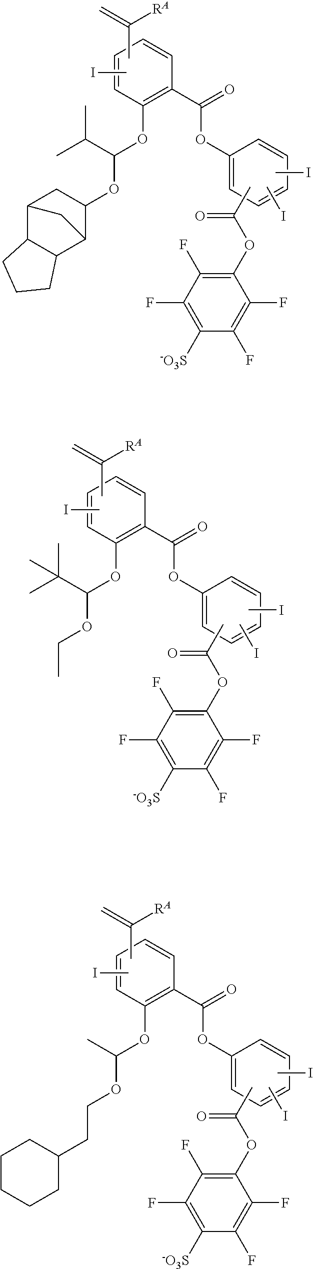

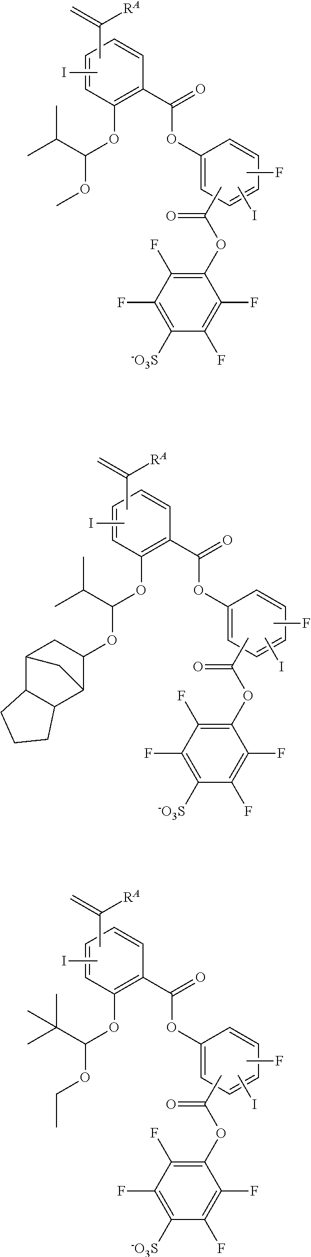

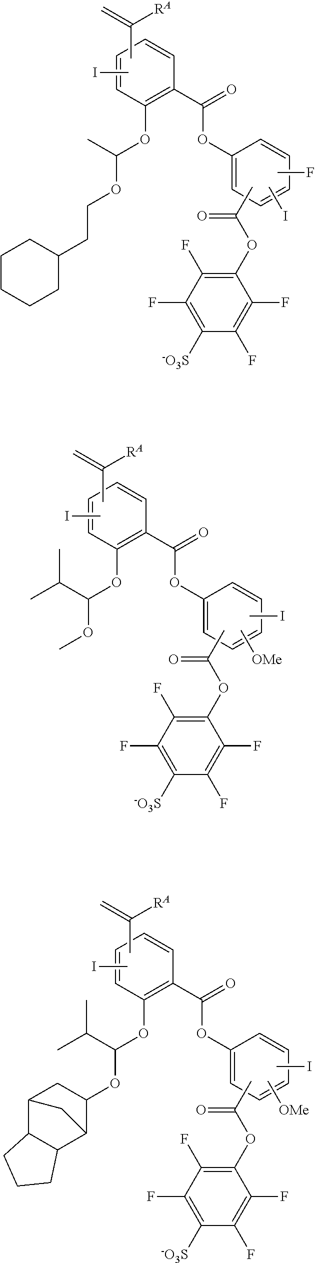

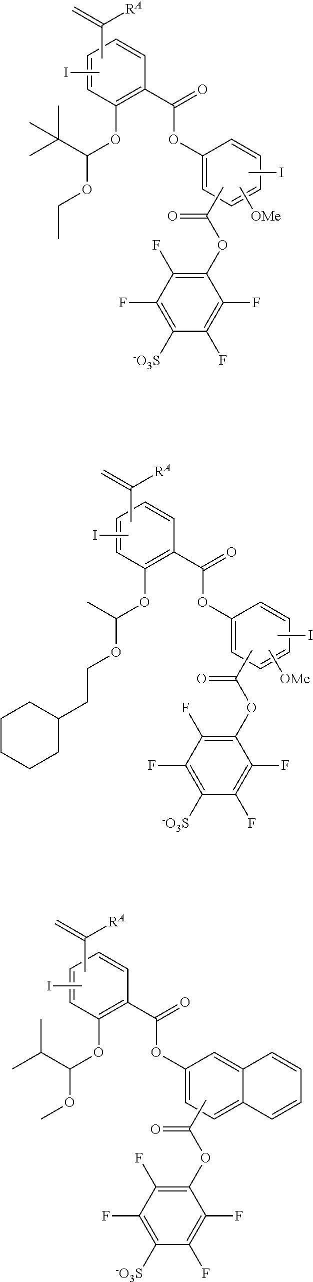

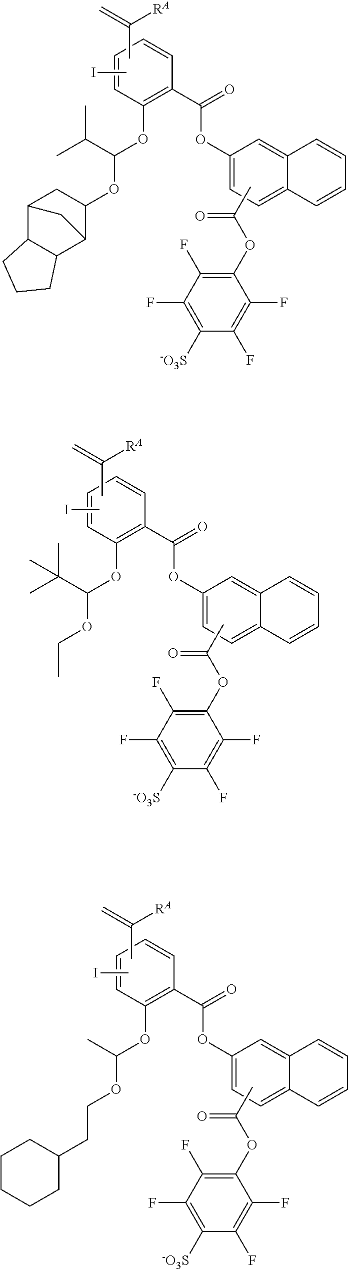

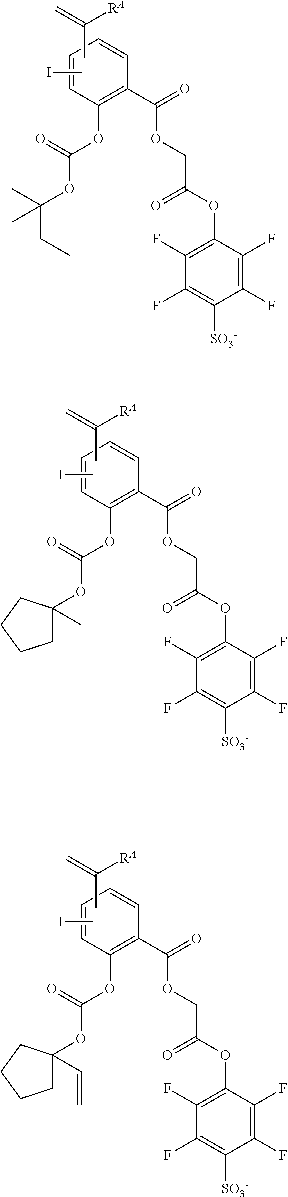

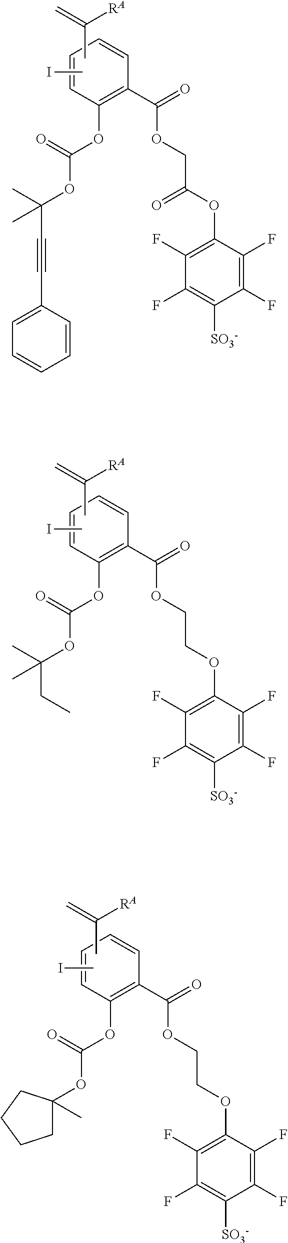

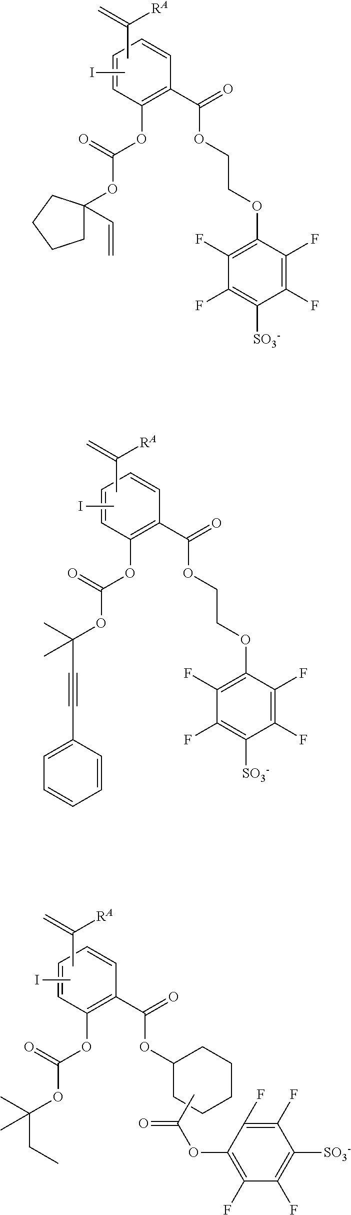

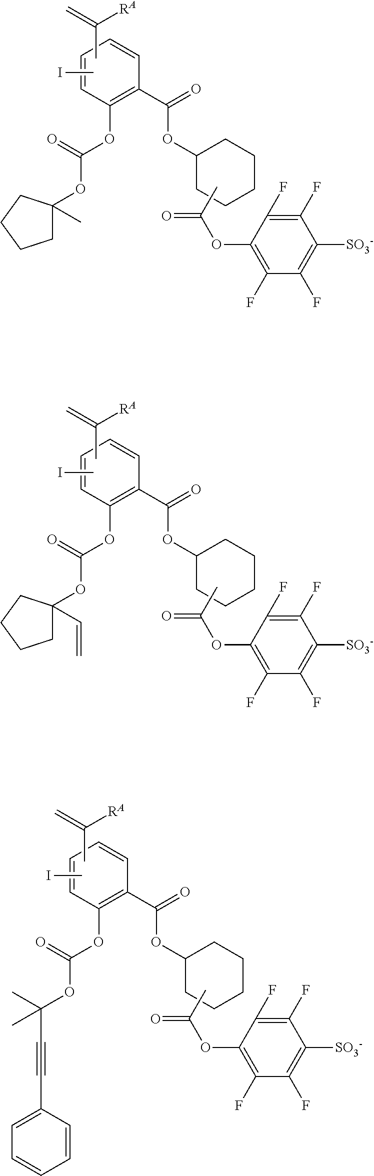

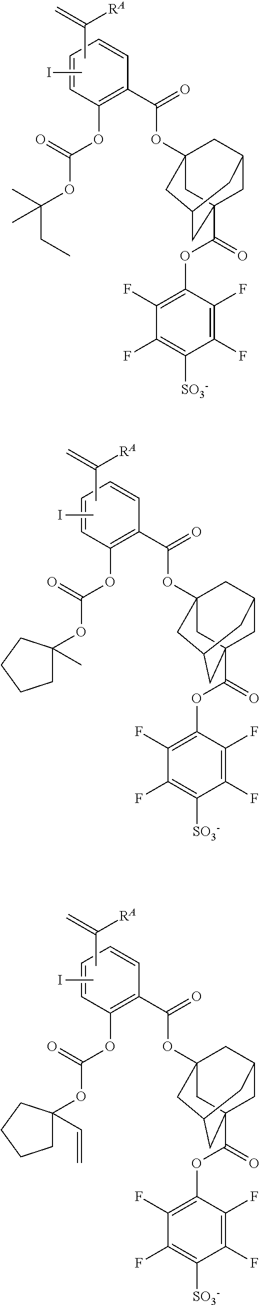

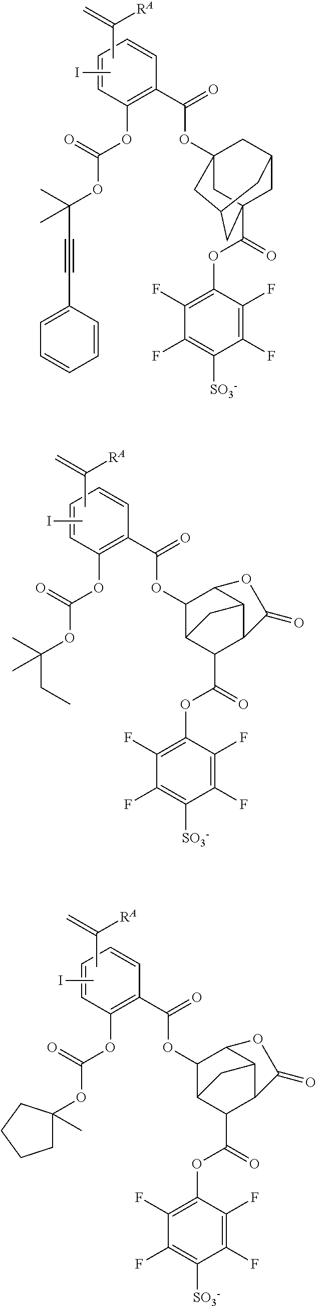

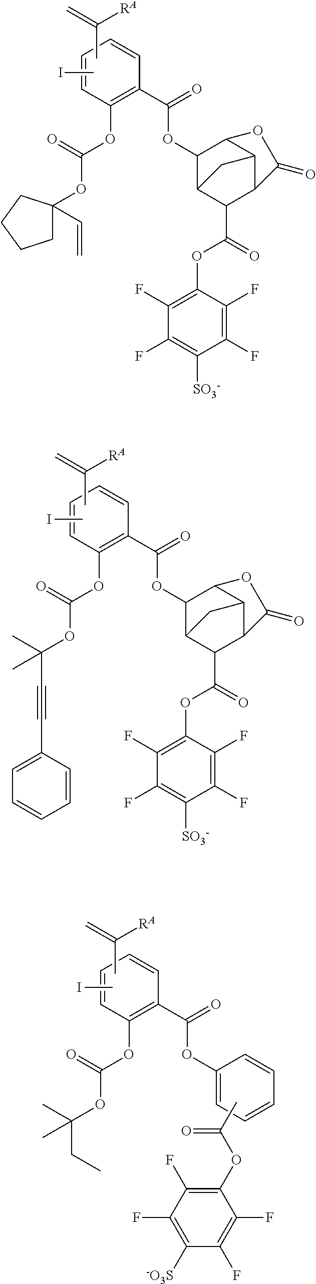

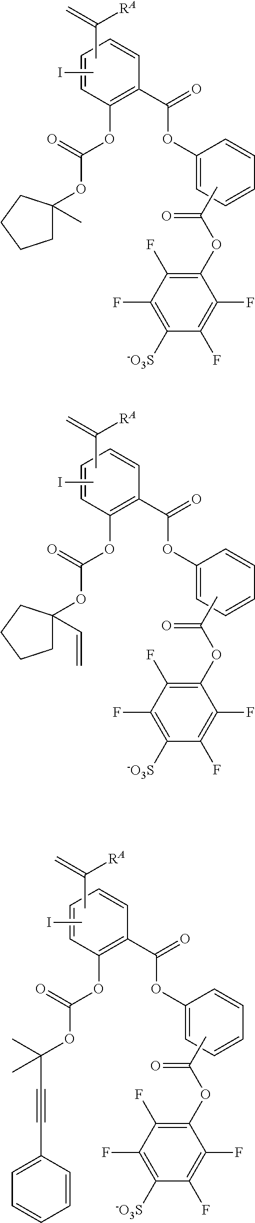

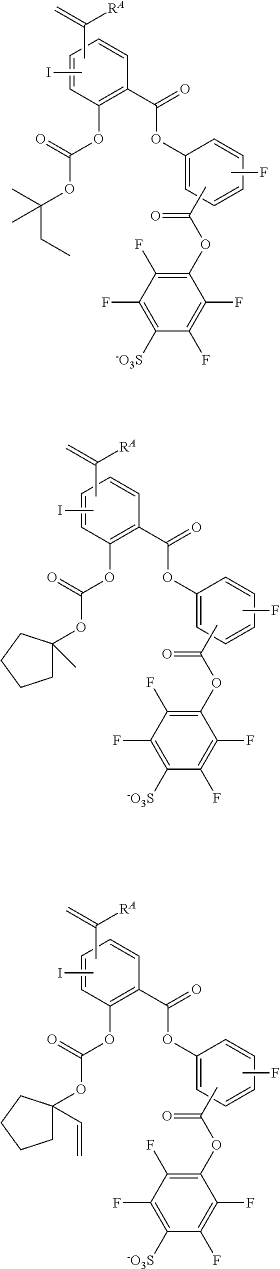

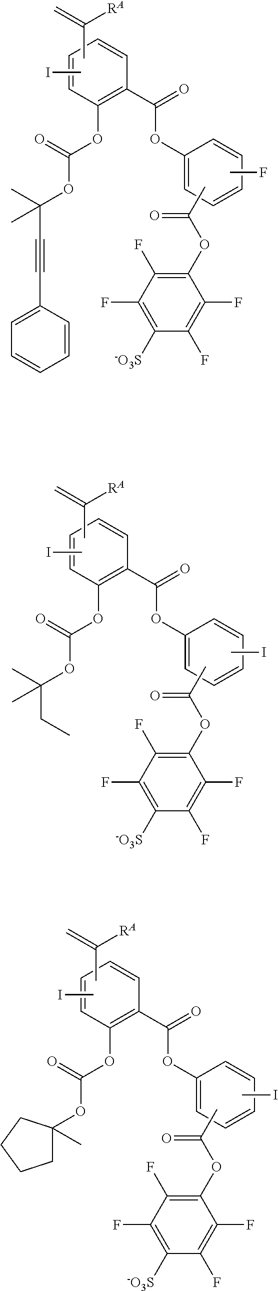

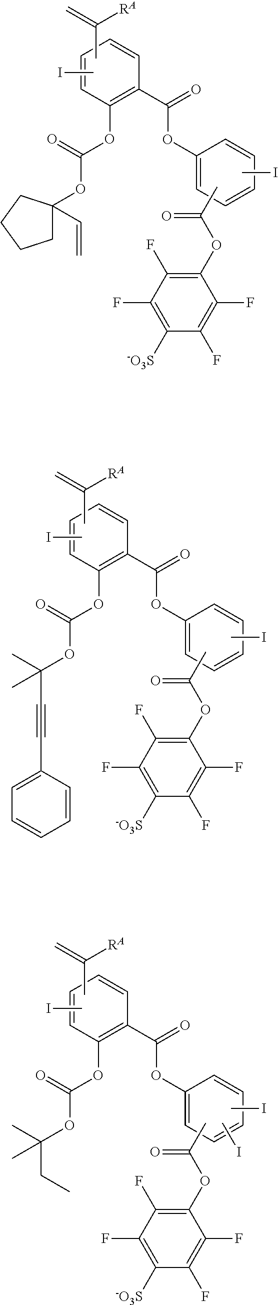

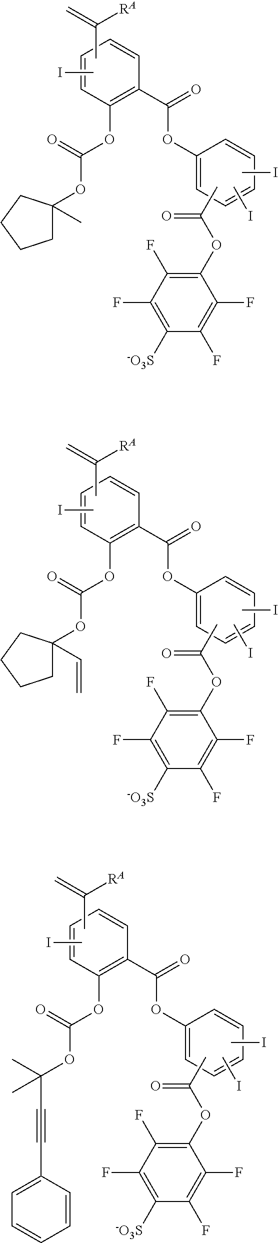

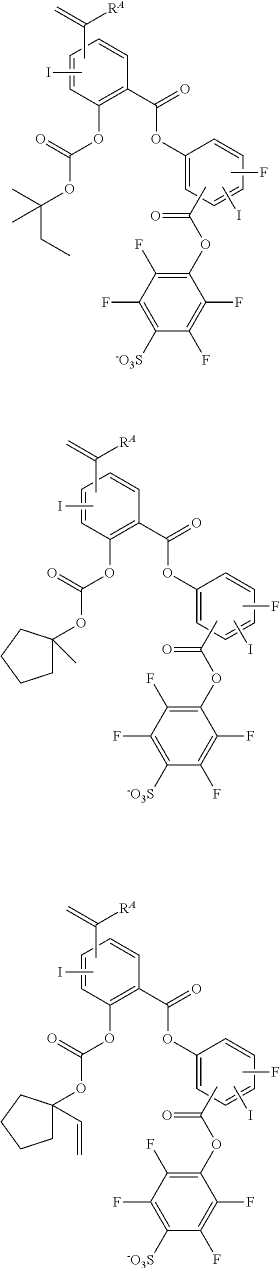

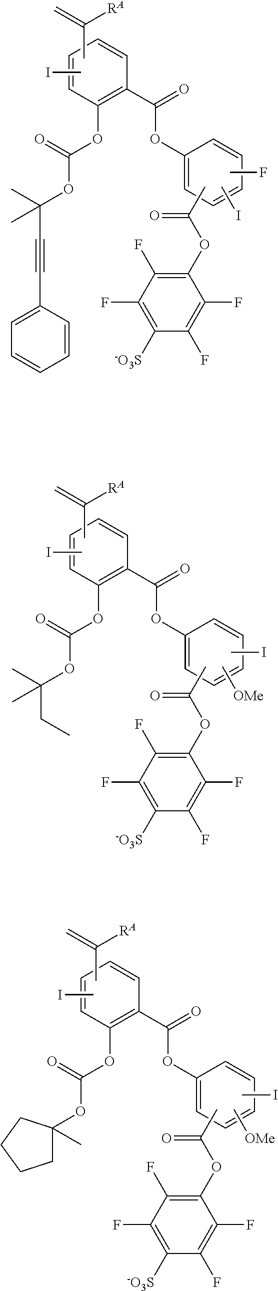

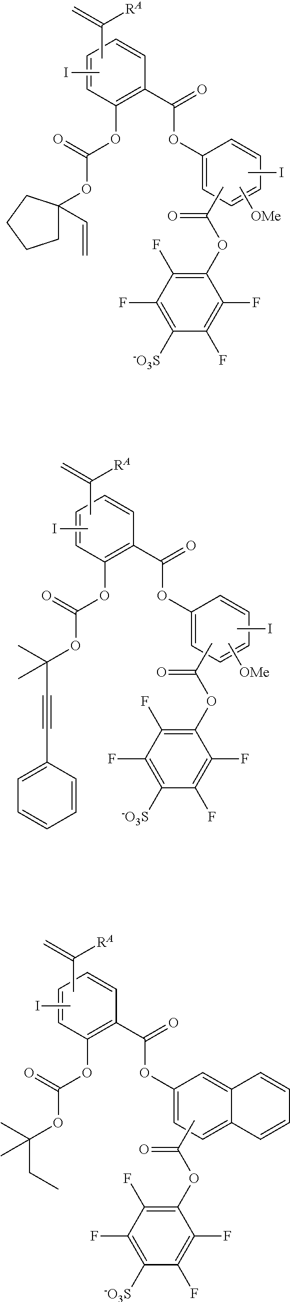

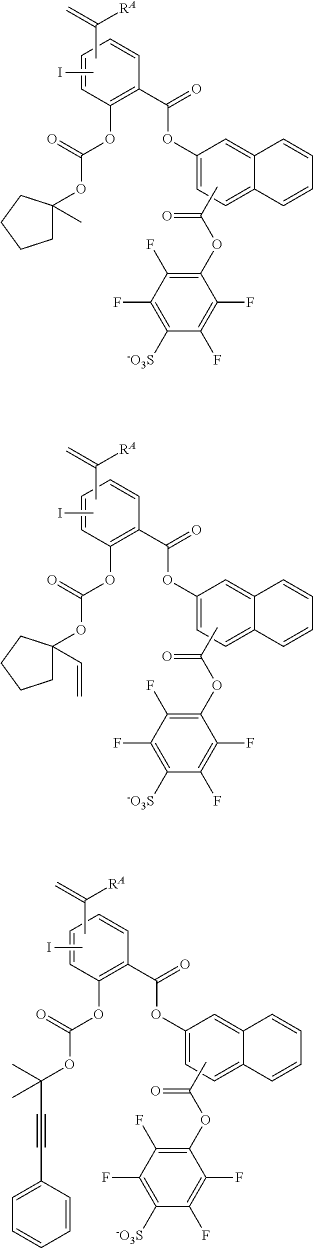

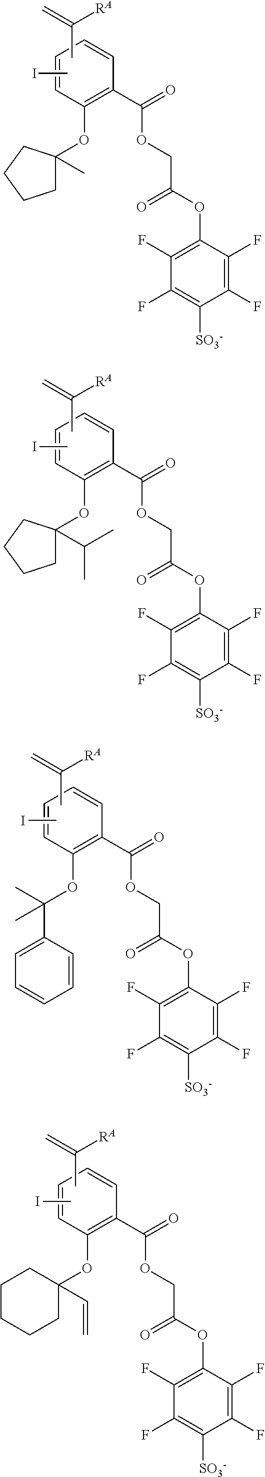

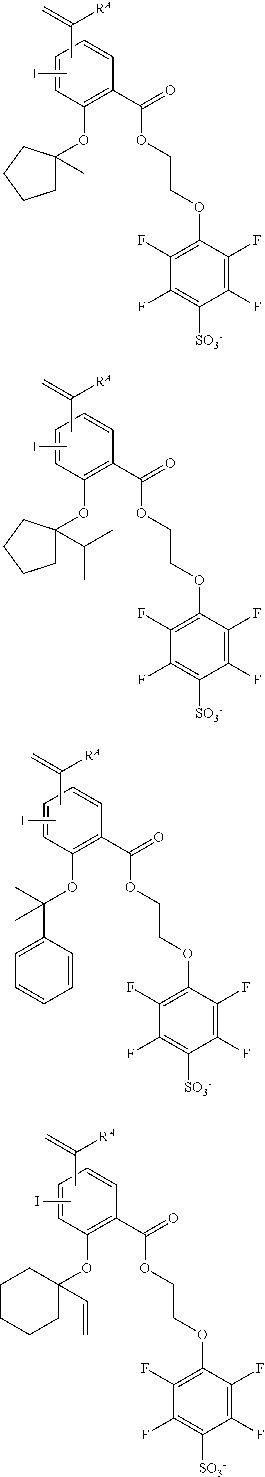

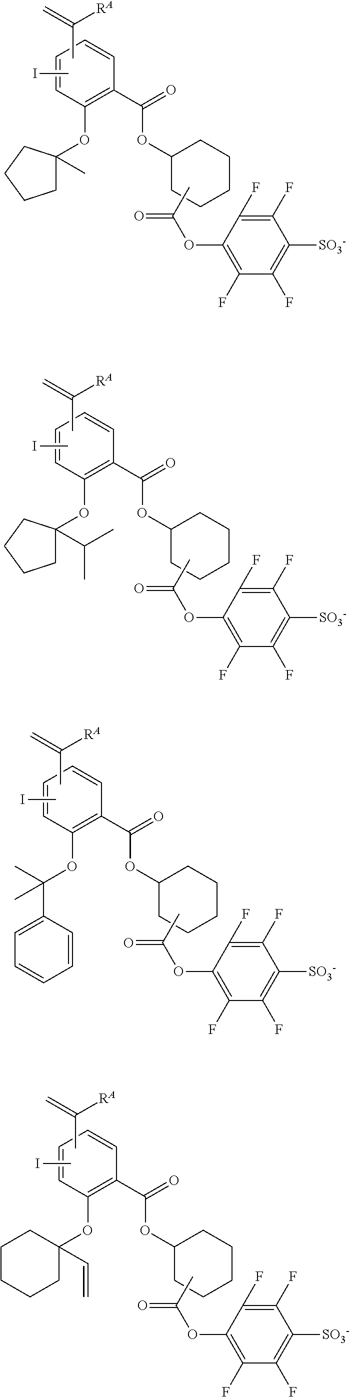

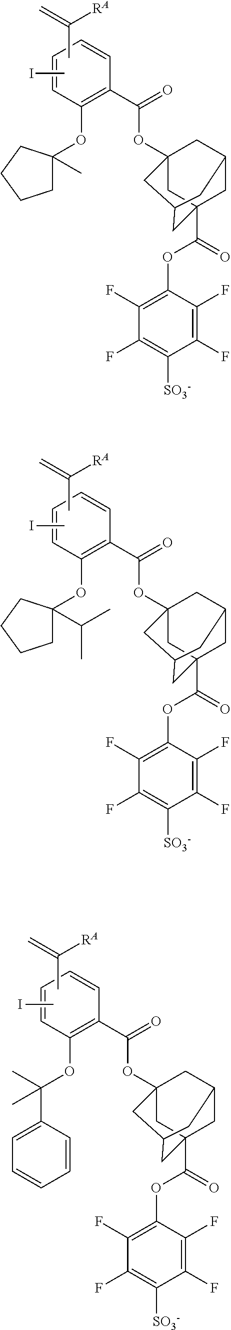

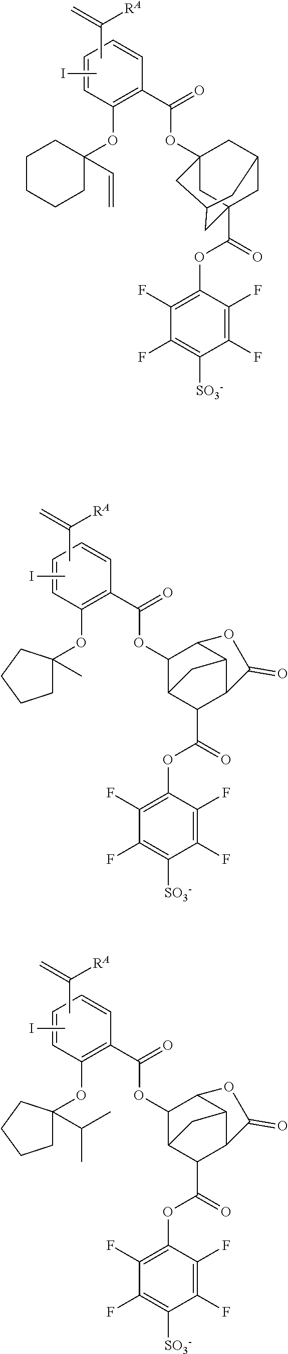

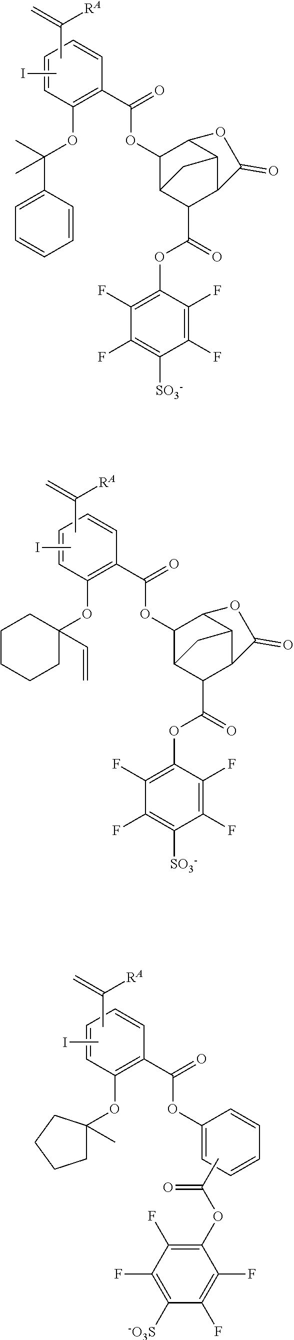

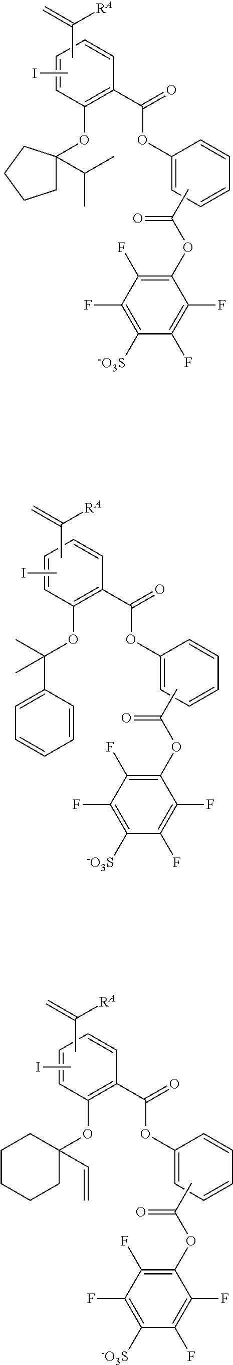

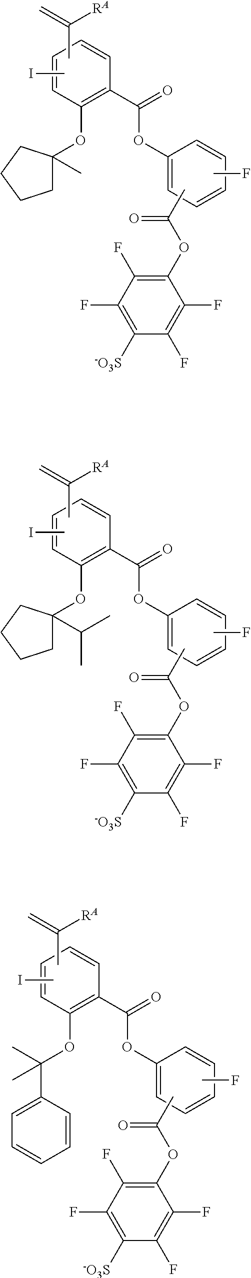

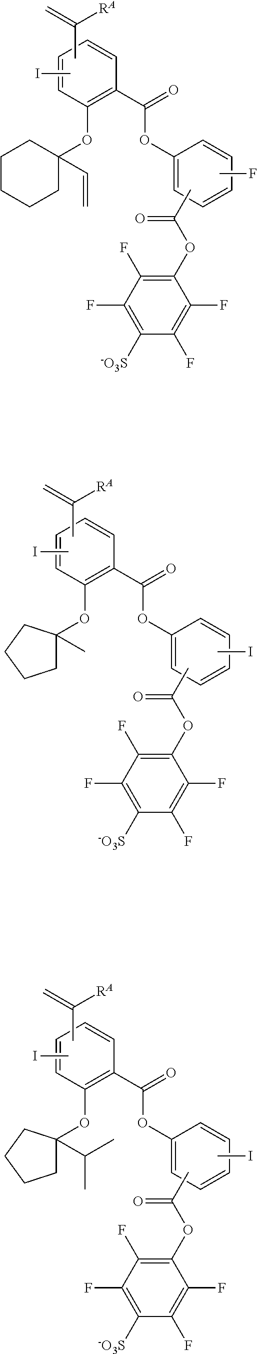

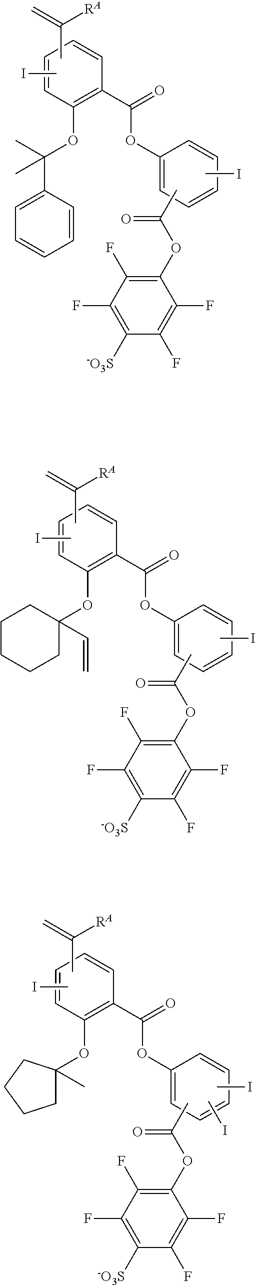

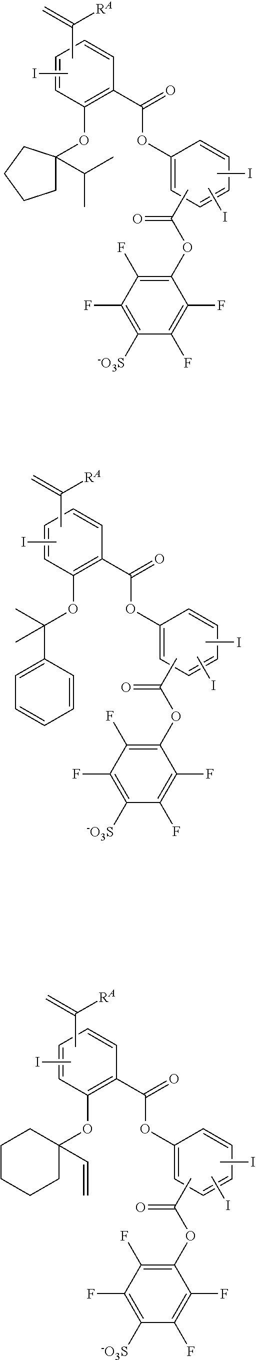

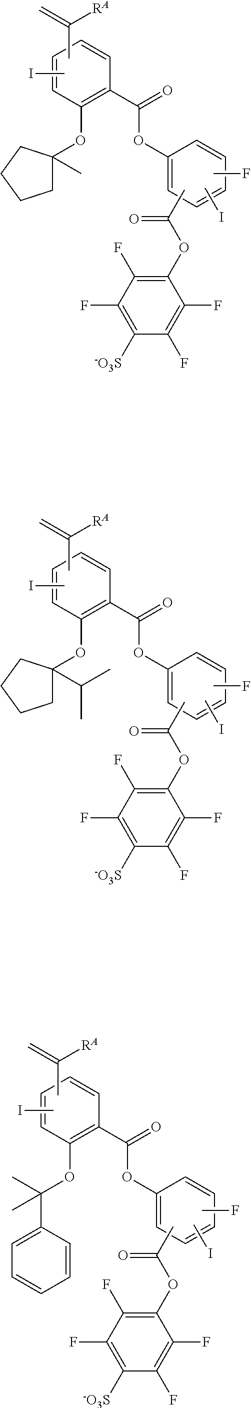

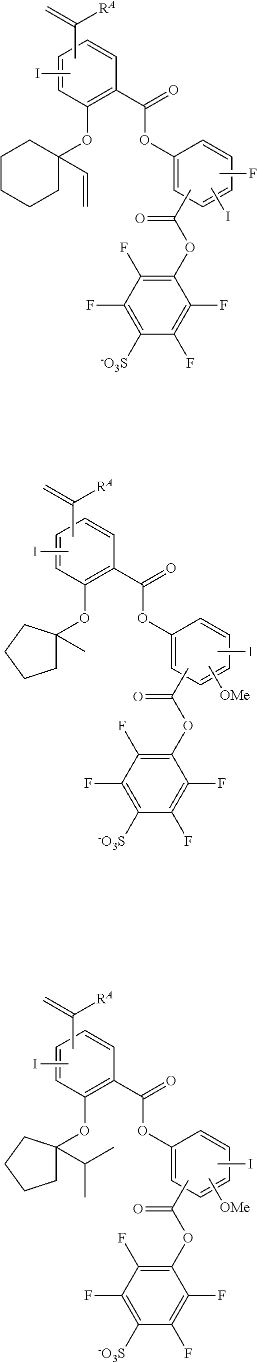

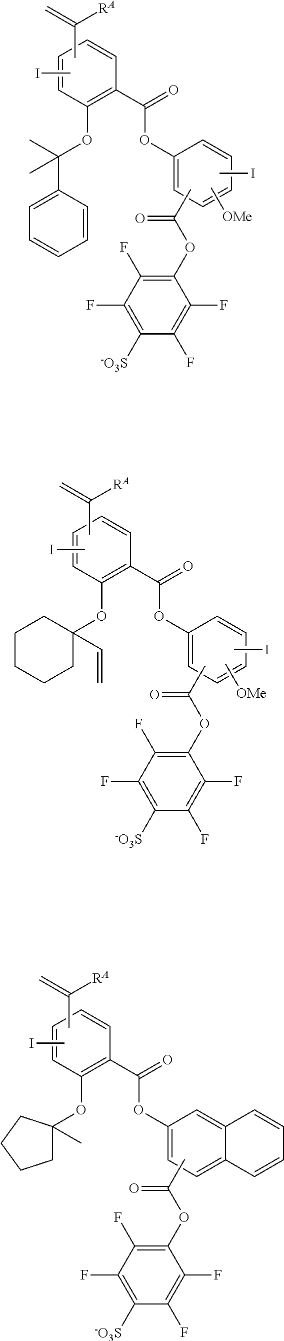

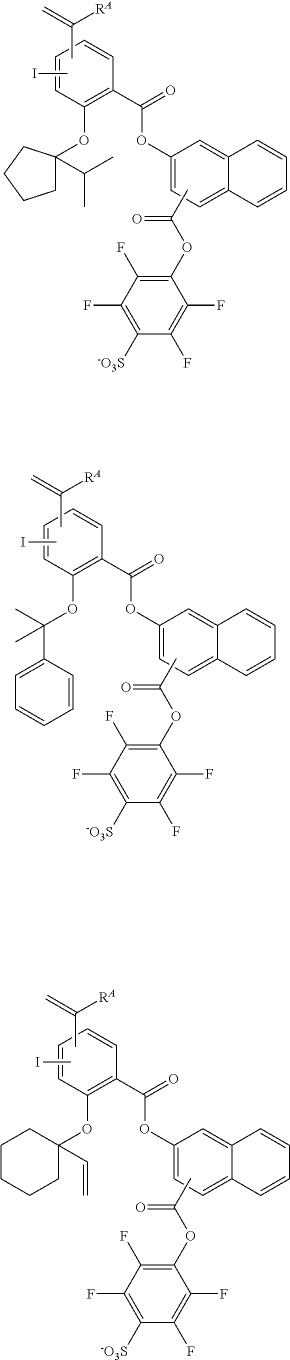

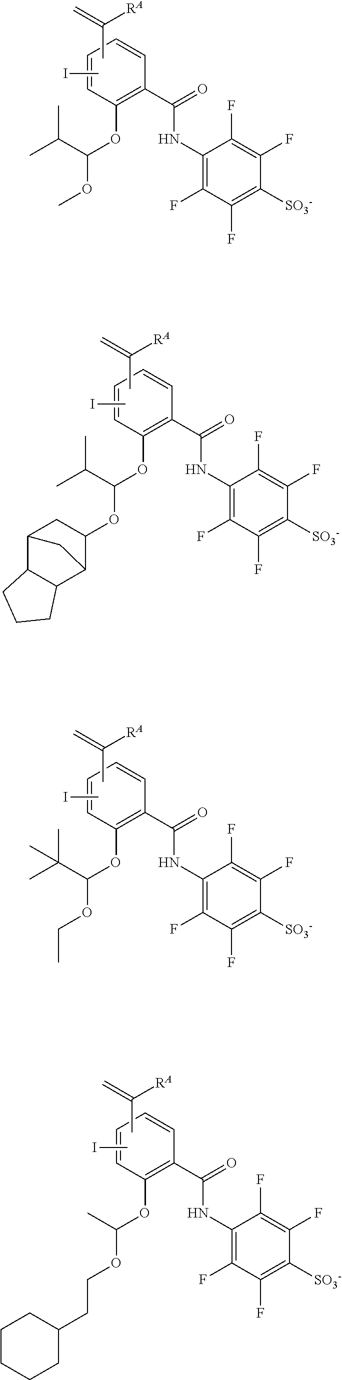

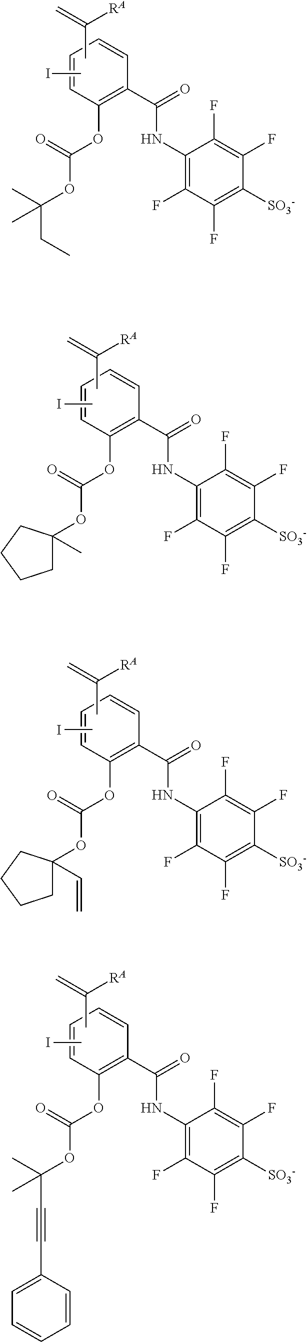

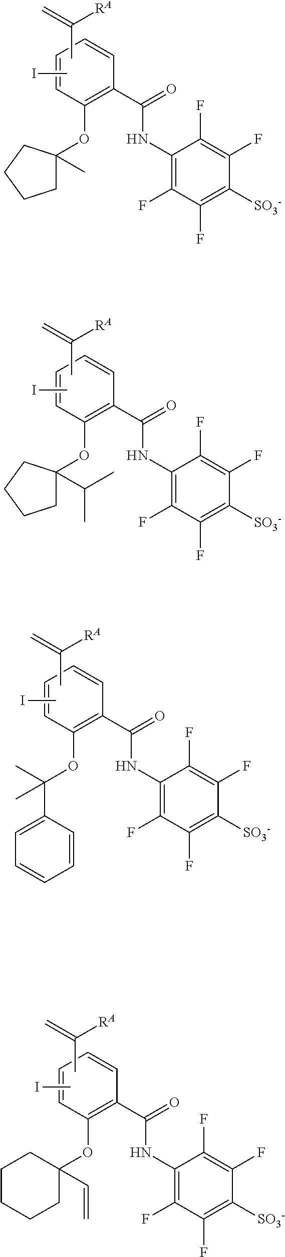

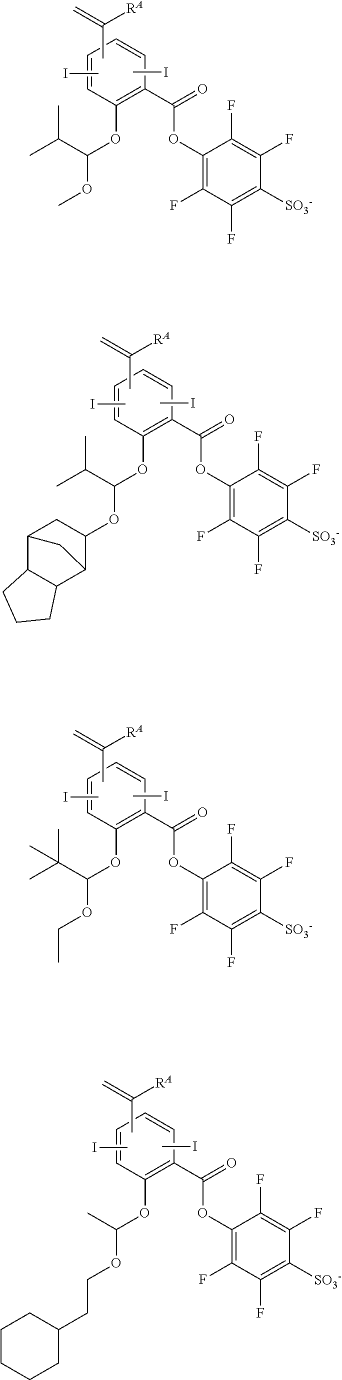

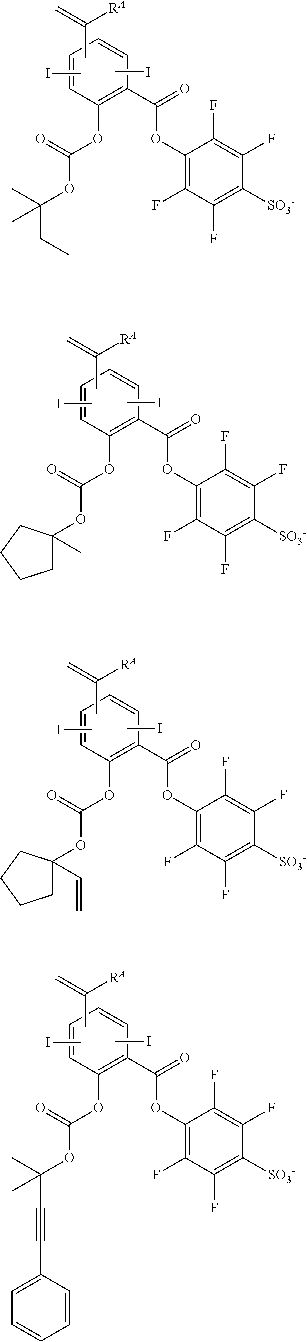

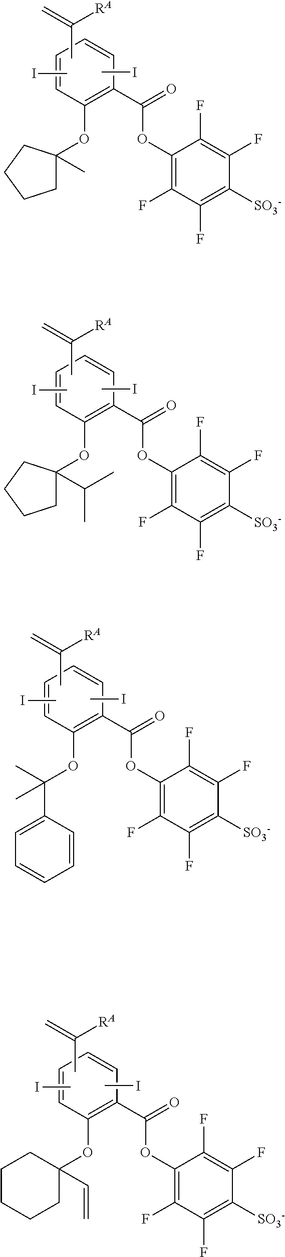

In the formula (a), Z⁺ represents an onium cation. As the onium cation, a sulfonium cation represented by the following formula (Z-1) or an iodonium cation represented by the following formula (Z-2) is preferable.

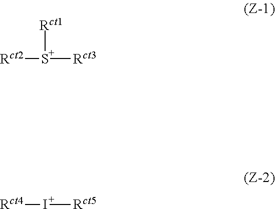

In the formulae (Z-1) and (Z-2), R^{ct1} to R^{ct5} each independently represent a halogen atom or a hydrocarbyl group having 1 to 30 carbon atoms and optionally containing a heteroatom.

Specific examples of the halogen atom represented by R^{ct1} to R^{ct5} include a fluorine atom, a chlorine atom, a bromine atom, and an iodine atom.

The hydrocarbyl group represented by R^{ct1} to R^{ct5} may be saturated or unsaturated, and may be linear, branched, or cyclic. Specific examples thereof include: alkyl groups having 1 to 30 carbon atoms, such as a methyl group, an ethyl group, an n-propyl group, an isopropyl group, an n-butyl group, an isobutyl group, a sec-butyl group, and a tert-butyl group; cyclic saturated hydrocarbyl groups having 3 to 30 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 30 carbon atoms, such as a vinyl group, an allyl group, a propenyl group, a butenyl group, and a hexenyl group; cyclic unsaturated hydrocarbyl groups having 3 to 30 carbon atoms, such as a cyclohexenyl group; aryl groups having 6 to 30 carbon atoms, such as a phenyl group, a naphthyl group, and a thienyl group; aralkyl groups having 7 to 30 carbon atoms, such as a benzyl group, a 1-phenylethyl group, and a 2-phenylethyl group; groups which are combinations of these groups; etc. Aryl groups are preferable. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, R^{ct1} and R^{ct2} may be bonded to each other to form a ring together with the sulfur atom bonded thereto. In this case, specific examples of the structure of the ring include those represented by the following formulae.

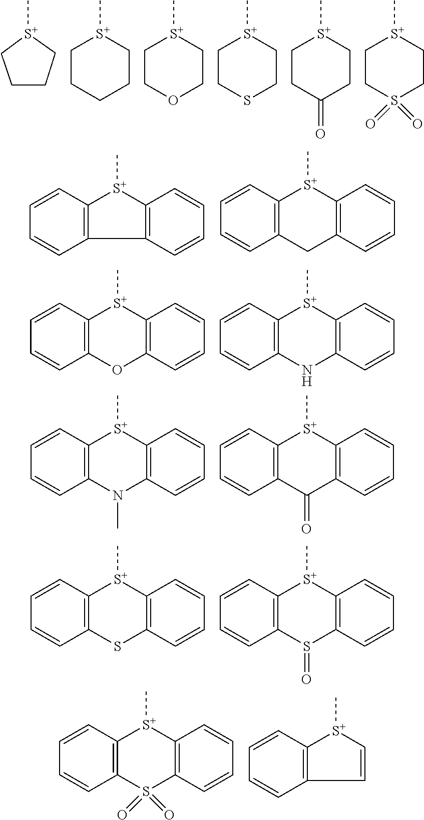

In the formulae, a broken line represents an attachment point to R^{ct3}.

Specific examples of the sulfonium cation represented by the formula (Z-1) include those disclosed in paragraphs [0102] to [0125] of JP 2024-003744 A and those disclosed in paragraphs [0070] to [0085] of JP 2023-169812 A, but are not limited thereto.

Specific examples of the iodonium cation represented by the formula (Z-2) include those disclosed in paragraph [0181] of JP 2024-000259 A, but are not limited thereto.

As the onium cation represented by Z⁺, sulfonium cations represented by the following formula (Z-3) are also favorable.

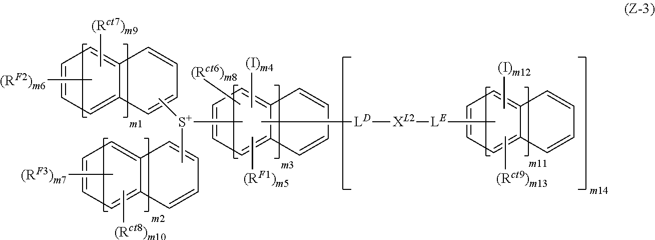

In the formula (Z-3), “m1” represents 0 or 1. When “m1” is 0, a benzene ring is indicated and when “m1” is 1, a naphthalene ring is indicated, and from the viewpoint of solvent solubility, a benzene ring, where “m1” is 0, is preferable. “m2” represents 0 or 1. When “m2” is 0, a benzene ring is indicated and when “m2” is 1, a naphthalene ring is indicated, and from the viewpoint of solvent solubility, a benzene ring, where “m2” is 0, is preferable. “m3” represents 0 or 1. When “m3” is 0, a benzene ring is indicated and when “m3” is 1, a naphthalene ring is indicated, and from the viewpoint of solvent solubility, a benzene ring, where “m3” is 0, is preferable.

In the formula (Z-3), “m4” represents 0, 1, 2, 3, or 4. The greater the number of iodine atoms in the cation structure, the higher the absorption of EUV, in particular, but the smaller the number of iodine atoms, the higher the solvent solubility and the lower the risk of precipitation in the resist composition. Therefore, “m4” is preferably 0, 1, 2, or 3, more preferably 0, 1, or 2.

In the formula (Z-3), “m5” represents 0, 1, 2, 3, or 4. From the viewpoint of procuring raw materials, “m5” is preferably 0, 1, 2, or 3, more preferably 0, 1, or 2. “m6” represents 0, 1, 2, 3, 4, 5, or 6. From the viewpoint of procuring raw materials, “m6” is preferably 0, 1, 2, or 3, more preferably 0, 1, or 2. “m7” represents 0, 1, 2, 3, 4, 5, or 6. From the viewpoint of procuring raw materials, “m7” is preferably 0, 1, 2, or 3, more preferably 0, 1, or 2.

In the formula (Z-3), “m8” represents 0, 1, or 2. From the viewpoint of procuring raw materials, “m8” is preferably 0 or 1. “m9” represents 0, 1, or 2. From the viewpoint of procuring raw materials, “m9” is preferably 0 or 1. “m10” represents 0, 1, or 2. From the viewpoint of procuring raw materials, “m10” is preferably 0 or 1.

In the formula (Z-3), “m11” represents 0 or 1. When “m11” is 0, a benzene ring is indicated and when “m11” is 1, a naphthalene ring is indicated, and from the viewpoint of solvent solubility, a benzene ring, where “m11” is 0, is preferable.

In the formula (Z-3), “m12” represents 0, 1, 2, 3, or 4. The greater the number of iodine atoms in the cation structure, the higher the absorption of EUV, in particular, but the smaller the number of iodine atoms, the higher the solvent solubility and the lower the risk of precipitation in the resist composition. Therefore, “m12” is preferably 0, 1, 2, or 3, more preferably 0, 1, or 2.

In the formula (Z-3), “m13” represents 0, 1, or 2. From the viewpoint of procuring raw materials, “m13” is preferably 0 or 1. “m14” represents 0, 1, or 2. From the viewpoint of synthesis, “m14” is preferably 0 or 1.

However, when “m1” is 0, 0 m6+m9≤4 and when “m1” is 1, 0 m6+m9≤6. When “m2” is 0, 0 m7+m10 K 4 and when “m2” is 1, 0 m7+m10≤6. When “m3” is 0, 1≤m4+m5+m8+m14≤4 and when “m3” is 1, 1 m4+m5+m8+m14≤6. When “m11” is 0, 0 m12+m13≤4 and when “m11” is 1, 0≤m12+m13≤6. In addition, m4+m12≤1.

In the formula (Z-3), R^{F1} to R^{F3} each independently represent a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms. Among these, a trifluoromethyl group, a trifluoromethoxy group, and a trifluorothiomethoxy group are preferable. When “m5” is 2 or more, the R^{F1}s are identical to or different from each other, when “m6” is 2 or more, the R^{F2}s are identical to or different from each other, and when “m7” is 2 or more, the R^{F3}s are identical to or different from each other.

In the formula (Z-3), R^{ct6} to R^{ct9} each represent a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom. The hydrocarbyl group and the hydrocarbyl moieties of the hydrocarbyloxy group and the hydrocarbylthio group may be saturated or unsaturated, and may be linear, branched, or cyclic. Specific examples thereof include those given as examples of the hydrocarbyl group represented by R¹ in the description of the formula (a). Furthermore, part or all of the hydrogen atoms of the hydrocarbyl group and the hydrocarbyl moieties of the hydrocarbyloxy group and the hydrocarbylthio group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, when “m8” is 2, the two R^{ct6}s are identical to or different from each other and the two R^{ct6}s are optionally bonded to each other to form a ring together with the carbon atoms bonded thereto, when “m9” is 2, the two R^{ct7}s are identical to or different from each other and the two R^{ct7}s are optionally bonded to each other to form a ring together with the carbon atoms bonded thereto, when “m10” is 2, the two R^{ct8}s are identical to or different from each other and the two R^{ct8}s are optionally bonded to each other to form a ring together with the carbon atoms bonded thereto, and when “m13” is 2, the two R^{ct9}s are identical to or different from each other and the two R^{ct9}s are optionally bonded to each other to form a ring together with the carbon atoms bonded thereto. Specific examples of the ring formed in this case include a cyclopropane ring, a cyclobutane ring, a cyclopentane ring, a cyclohexane ring, a norbornane ring, and an adamantane ring. Furthermore, part or all of the hydrogen atoms in the ring may be substituted with a group containing a heteroatom, such as an oxygen atom, a sulfur atom, a nitrogen atom, or a halogen atom, and part of the —CH₂— in the ring may be substituted with a group containing a heteroatom such as an oxygen atom, a sulfur atom, or a nitrogen atom. The resulting ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, the aromatic rings bonded directly to the S⁺ in the sulfonium cation represented by the formula (Z-3) are optionally bonded to each other to form a ring together with the S⁺. In this case, specific examples of the structure of the ring include those represented by the following formulae.

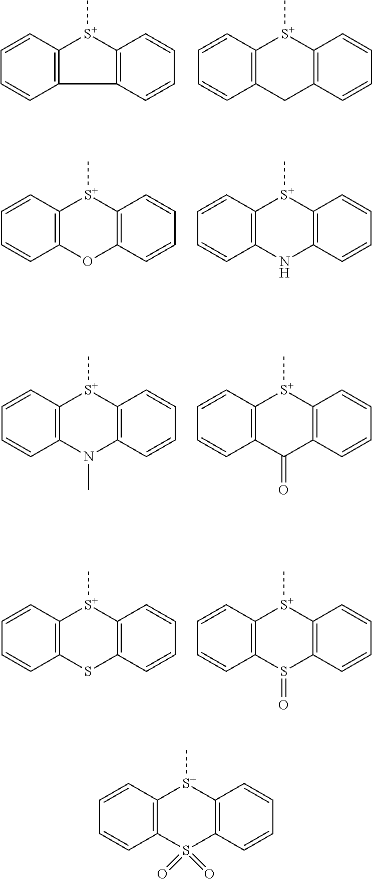

In the formulae, a broken line represents an attachment point.

In the formula (Z-3), L^{D} and L^{E} each independently represent a single bond, an ether bond, an ester bond, an amide bond, a sulfonic acid ester bond, a sulfonic acid amide bond, a carbonate bond, or a carbamate bond. Among these, L^{D} is preferably a single bond, an ether bond, an ester bond, or a sulfonic acid ester bond, more preferably an ester bond or a sulfonic acid ester bond. L^{E} is preferably a single bond, an ether bond, or an ester bond, more preferably a single bond.

In the formula (Z-3), X^{L2} represents a single bond or a hydrocarbylene group having 1 to 40 carbon atoms and optionally containing a heteroatom. Specific examples of the hydrocarbylene group having 1 to 40 carbon atoms and optionally containing a heteroatom include those given as specific examples of the hydrocarbylene group represented by X^{L} having 1 to 40 carbon atoms and optionally containing a heteroatom, but are not limited thereto.

As the sulfonium cation represented by the formula (Z-3), one represented by the following formula (Z-3-1) is preferable.

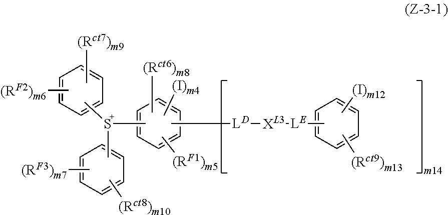

In the formula, “m4” to “m10”, “m12” to “m14”, R^{F1} to R^{F3}, R^{ct6} to R^{ct9}, L^{D}, L^{E}, and X^{L2} are as defined above.

As the cation represented by the formula (Z-3-1), one represented by the following formula (Z-3-2) is preferable.

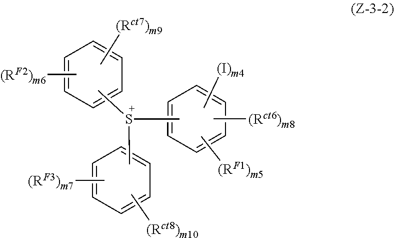

In the formula, “m4” to “m10”, R^{F1} to R^{F3}, and R^{ct6} to R^{ct8} are as defined above.

Specific examples of the sulfonium cation represented by the formula (Z-3) include the following, but are not limited thereto. Note that, in the following formulae, Me represents a methyl group.

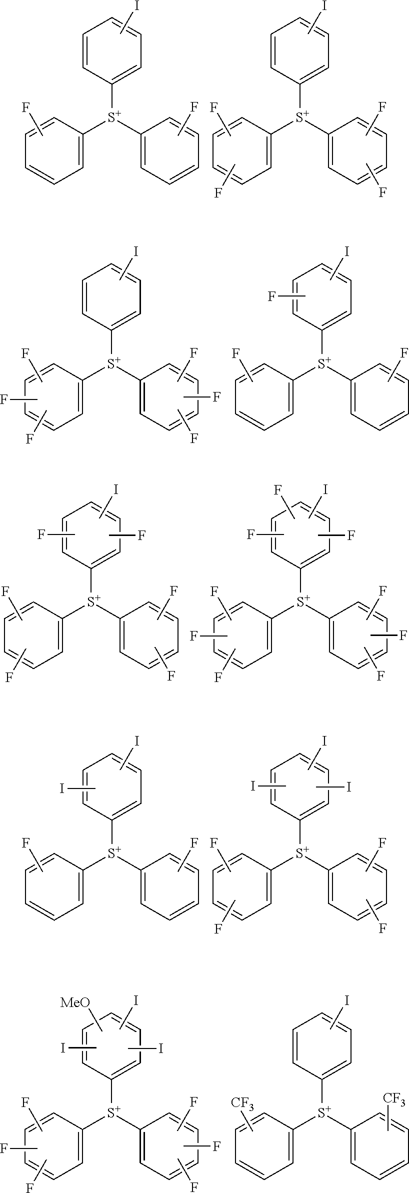

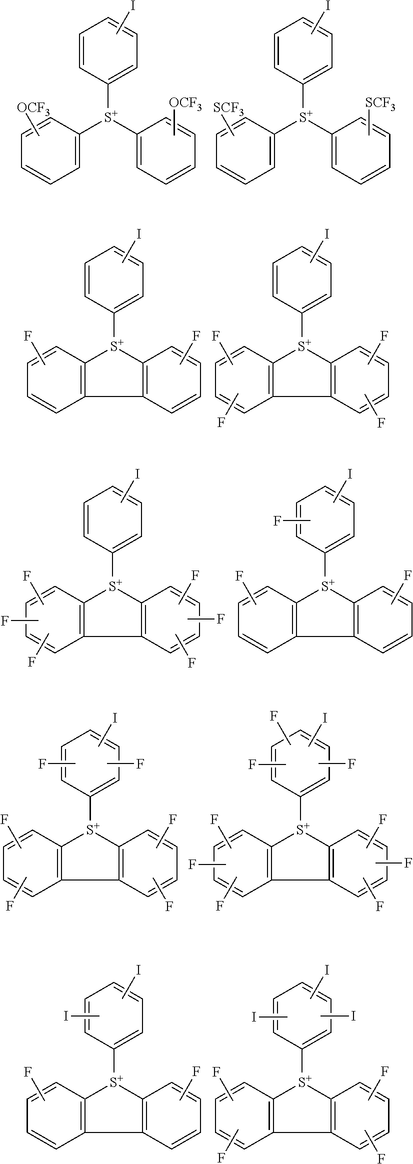

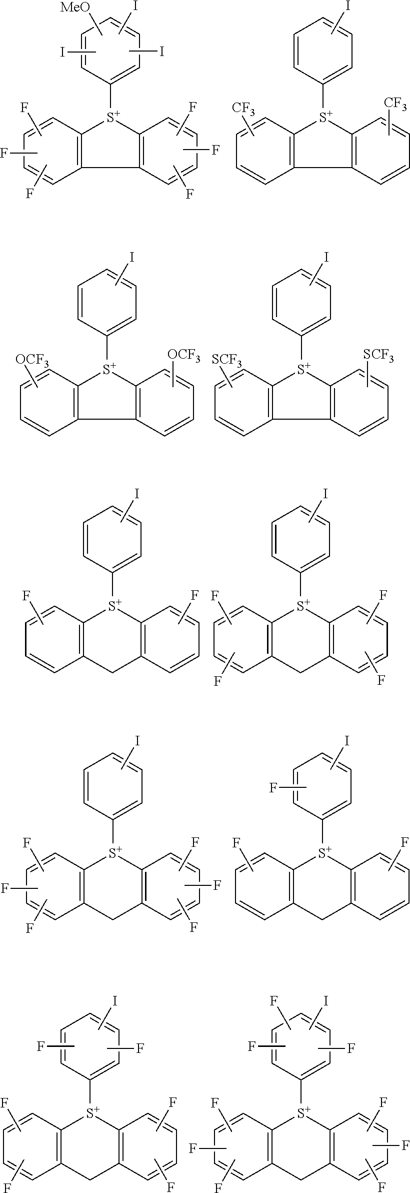

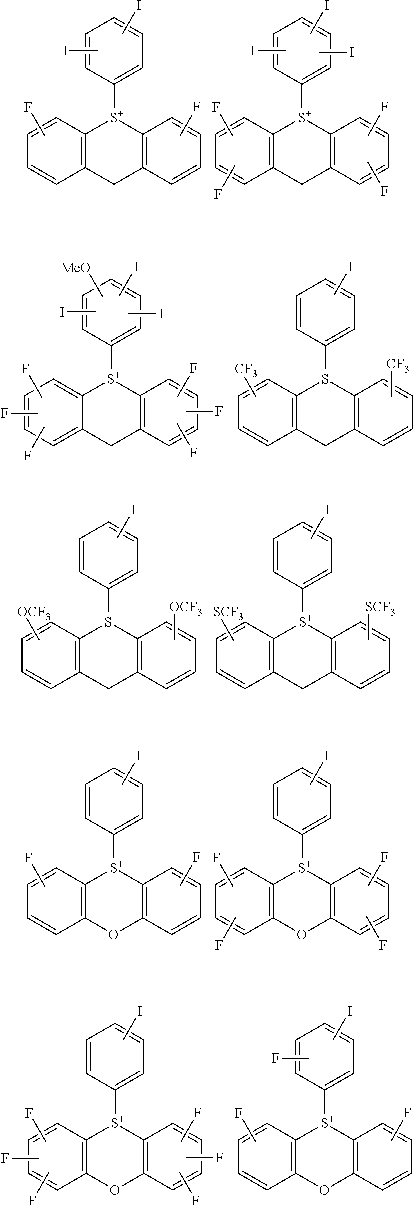

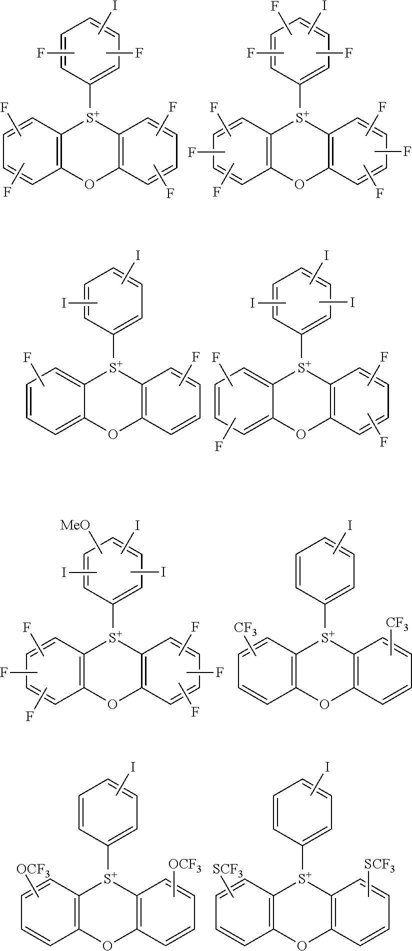

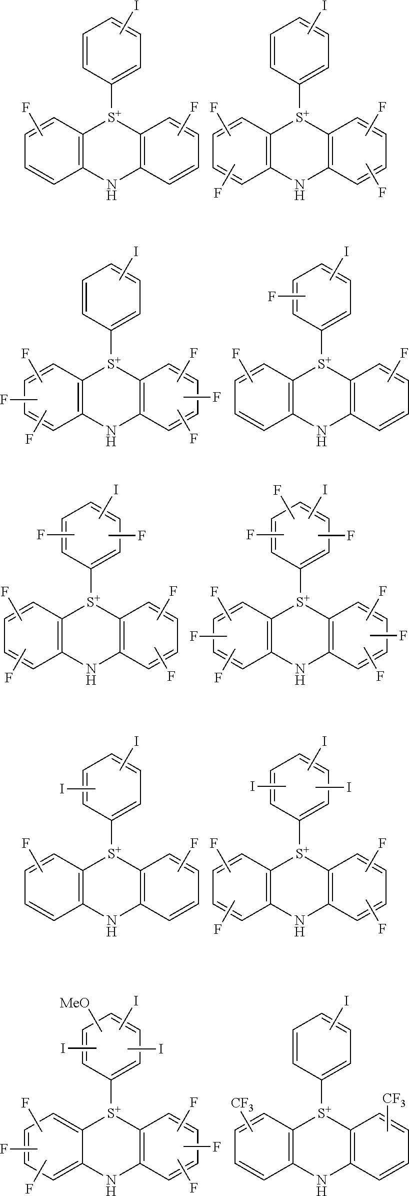

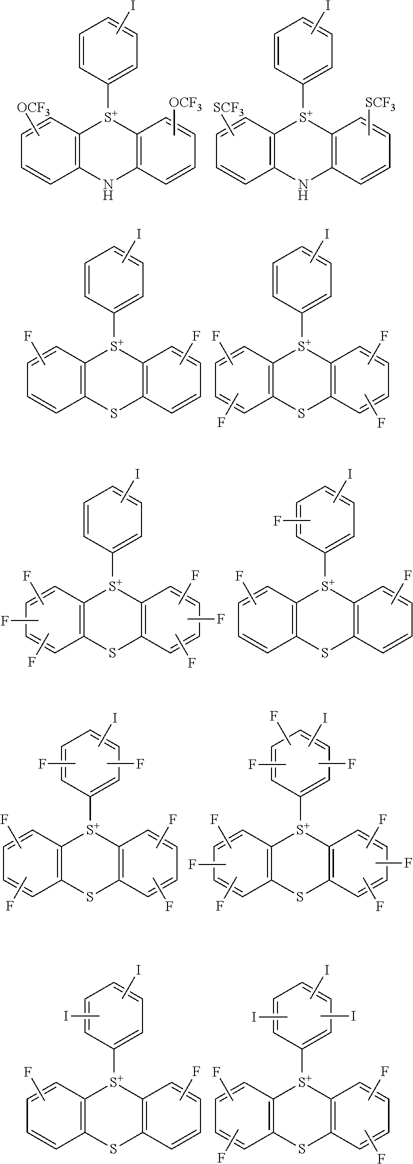

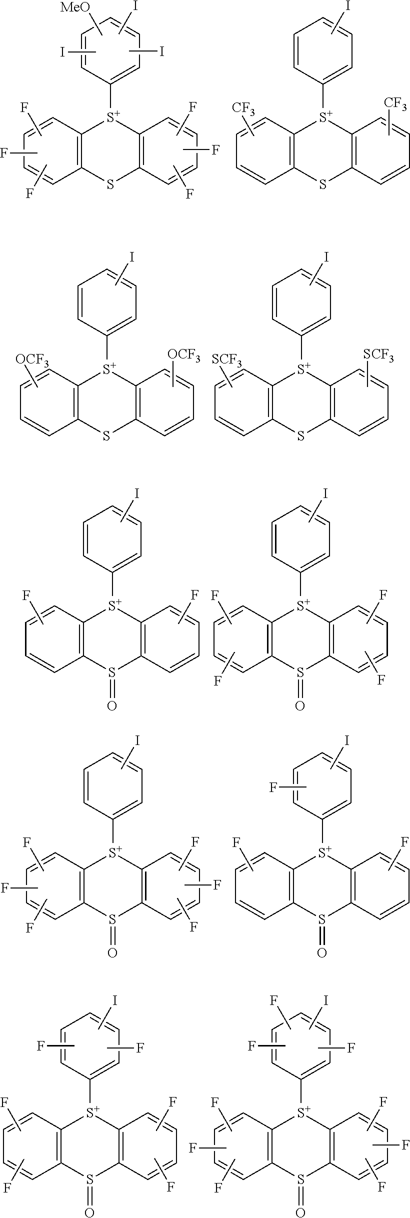

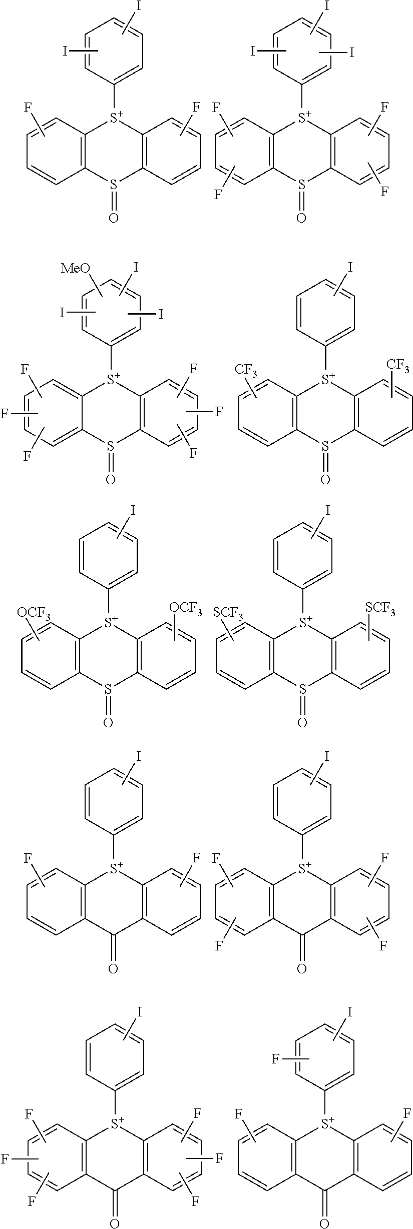

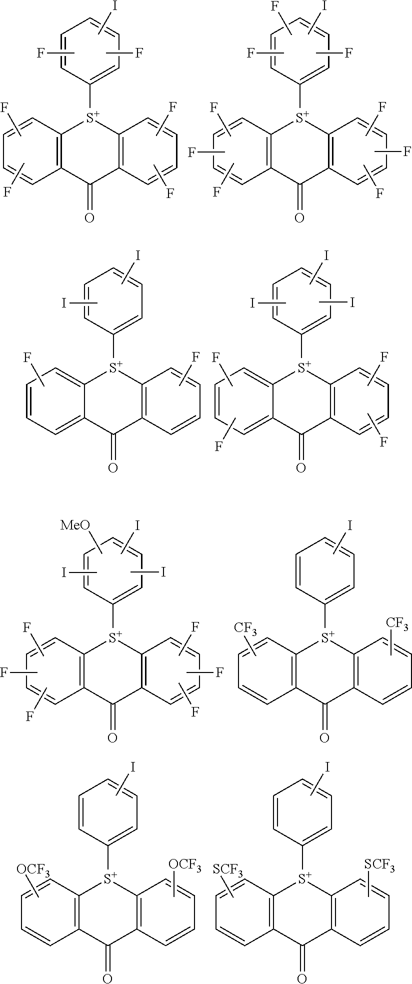

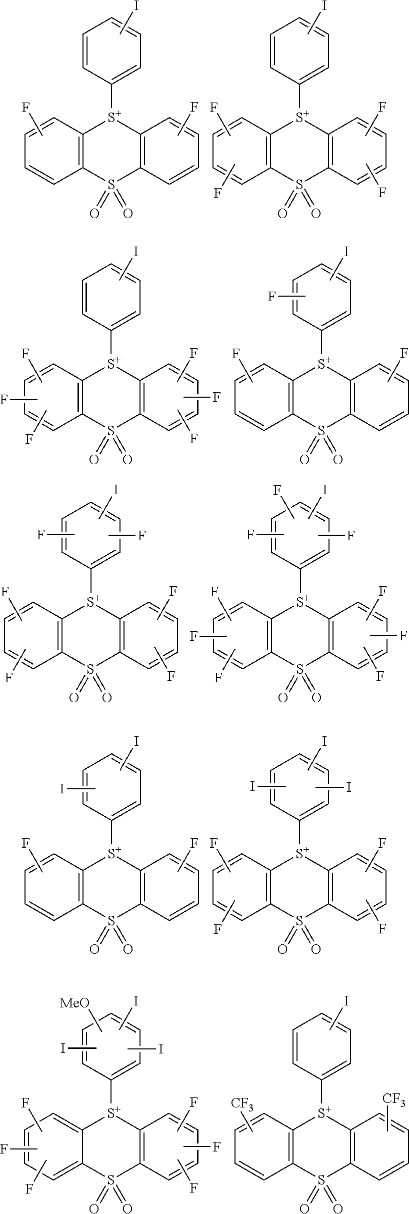

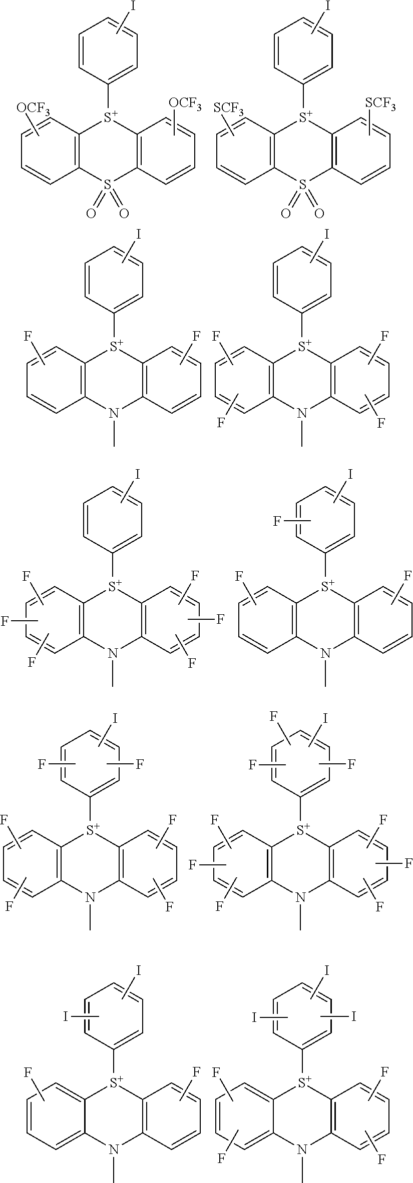

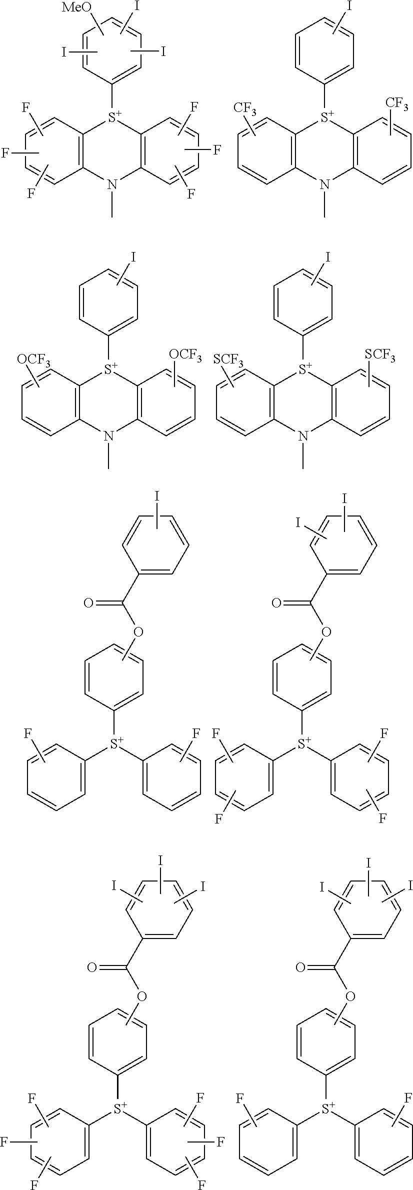

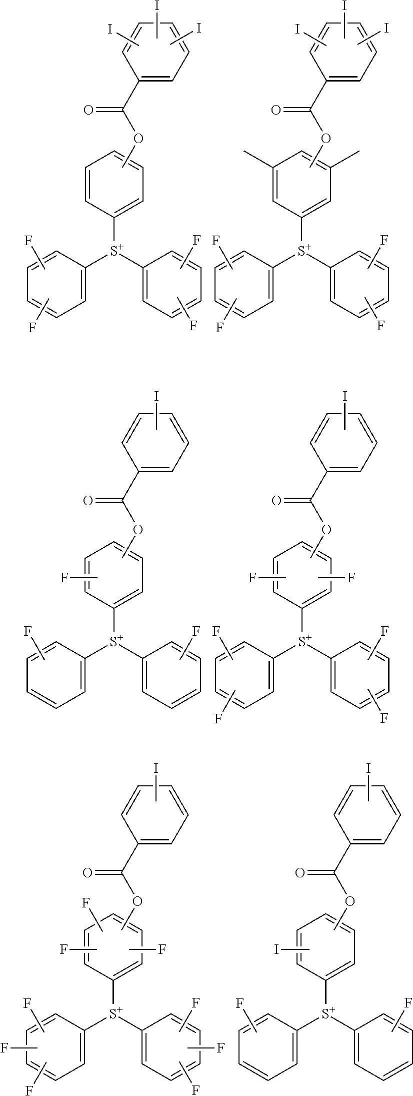

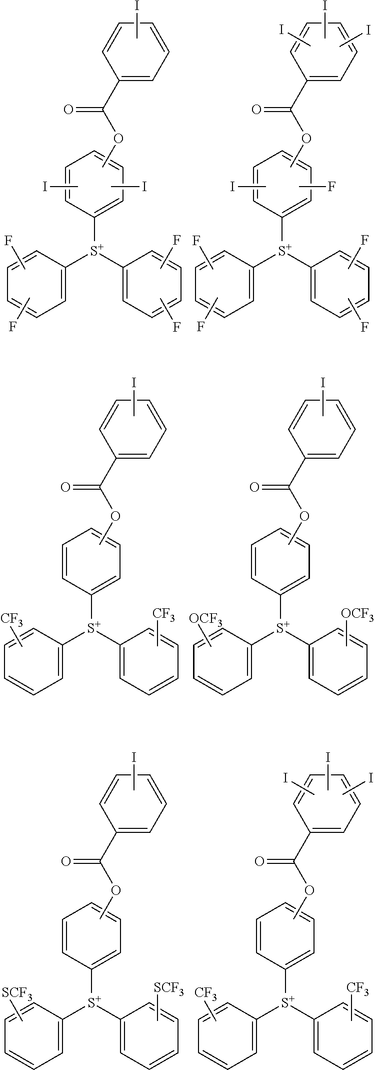

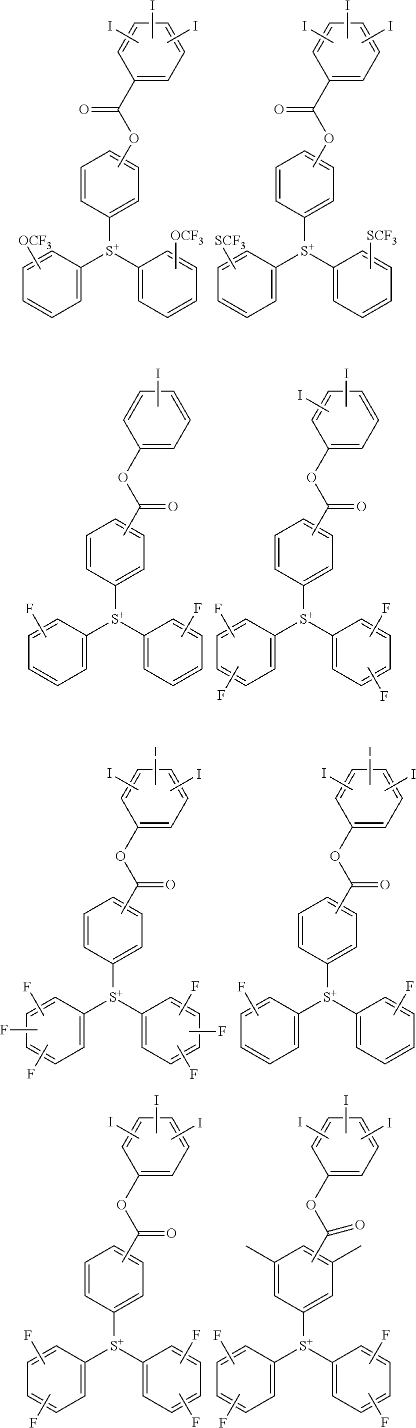

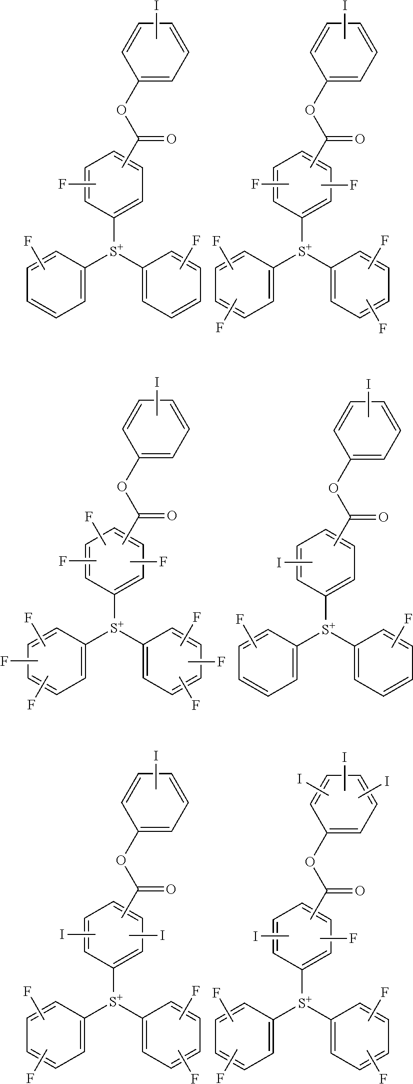

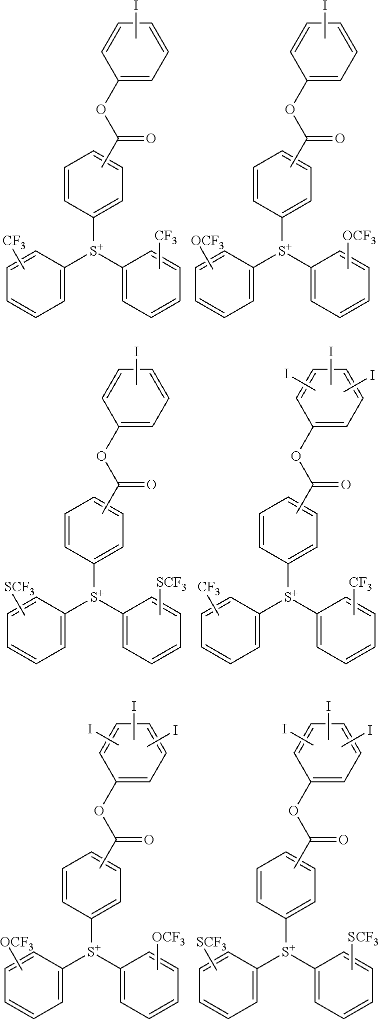

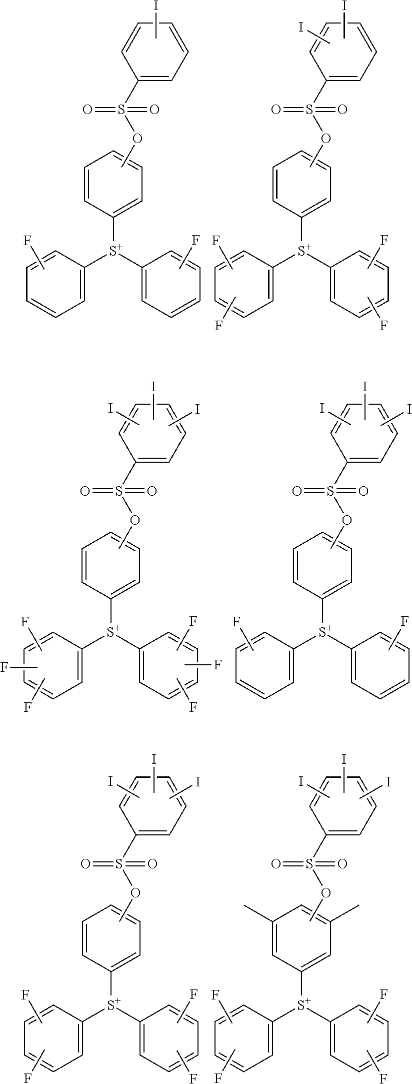

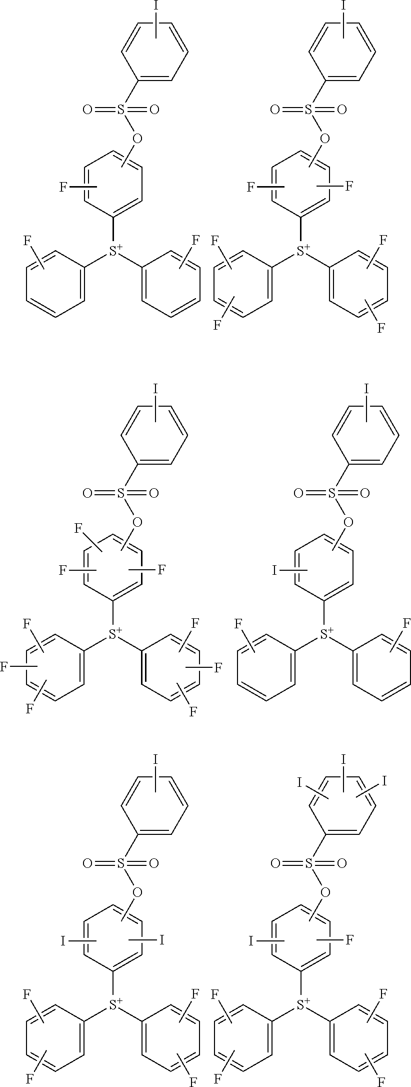

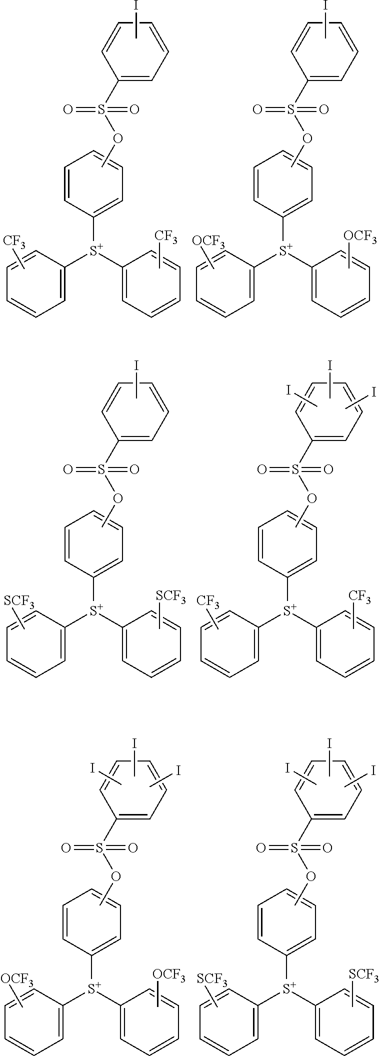

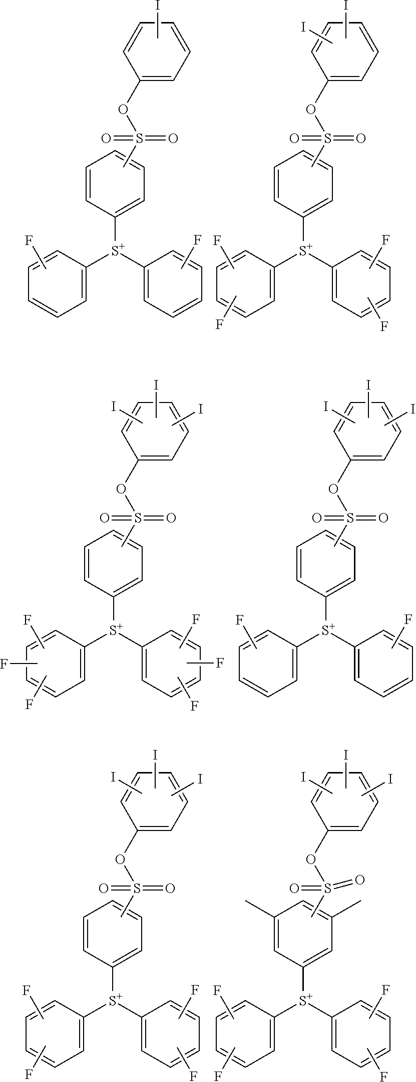

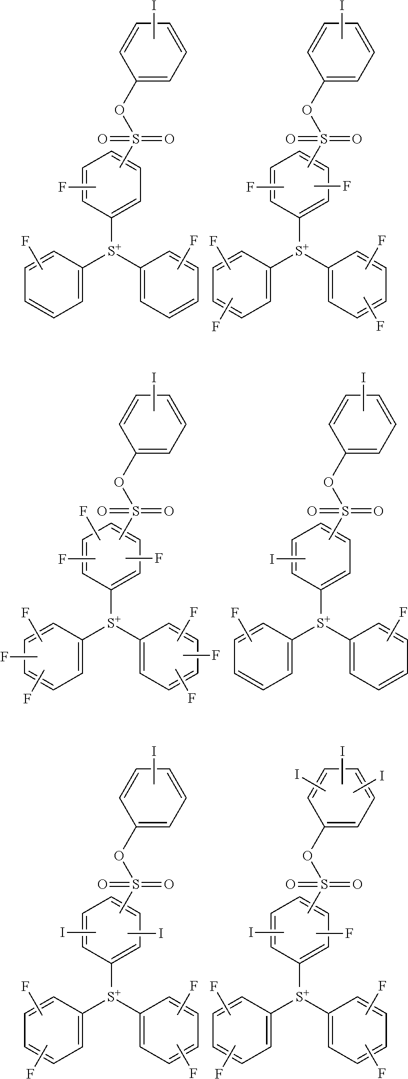

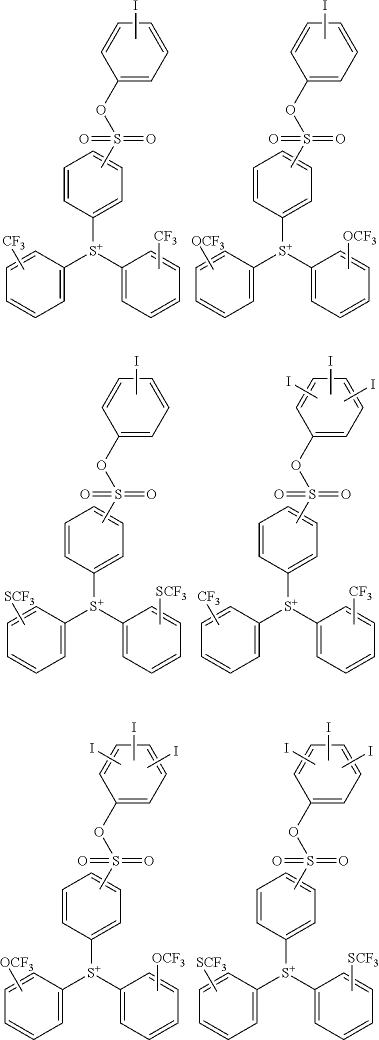

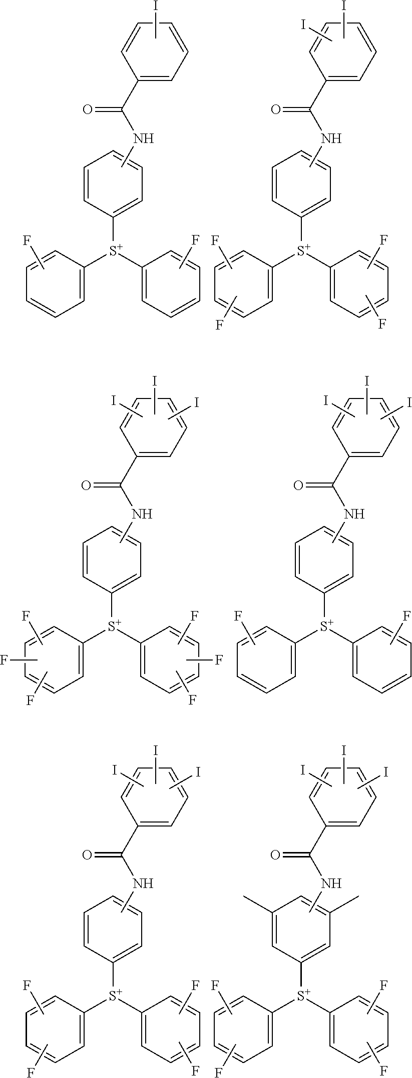

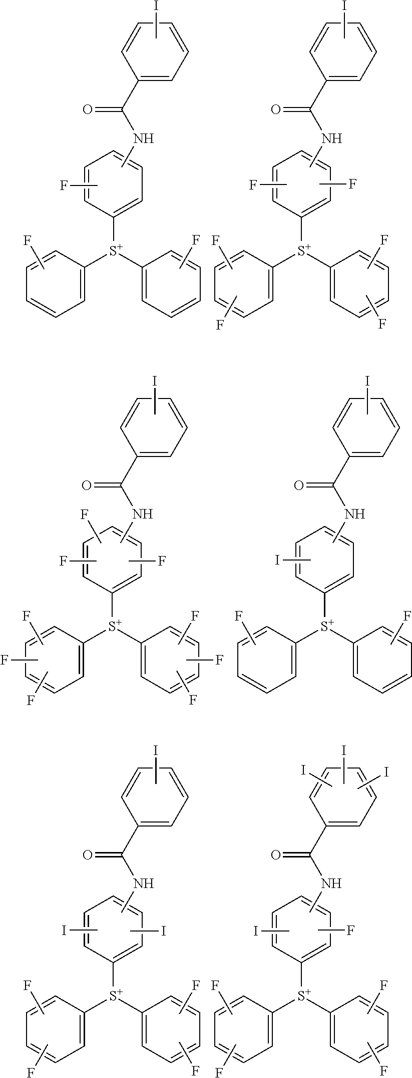

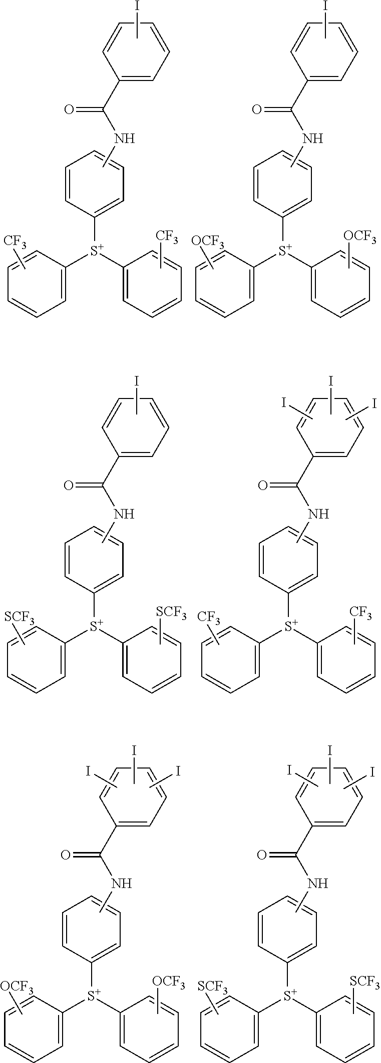

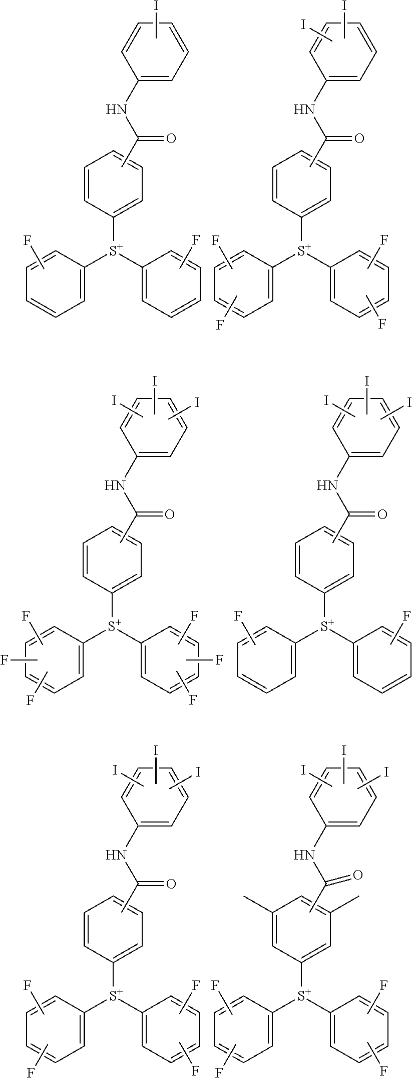

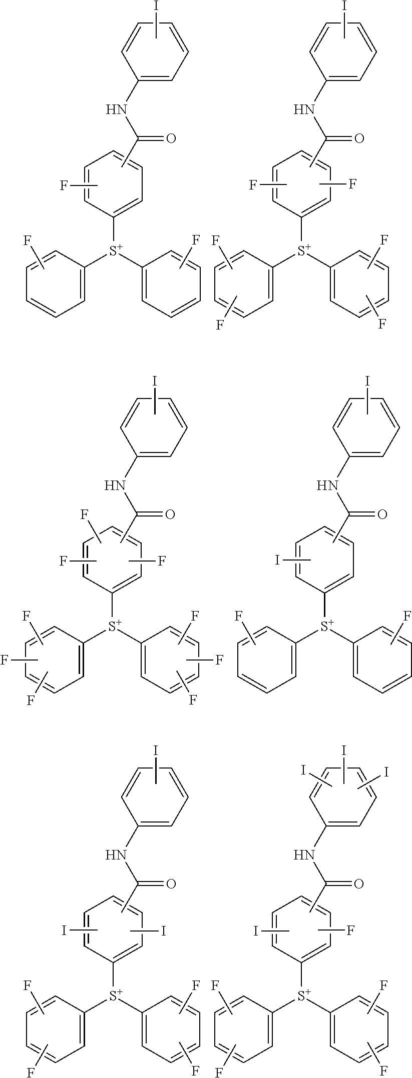

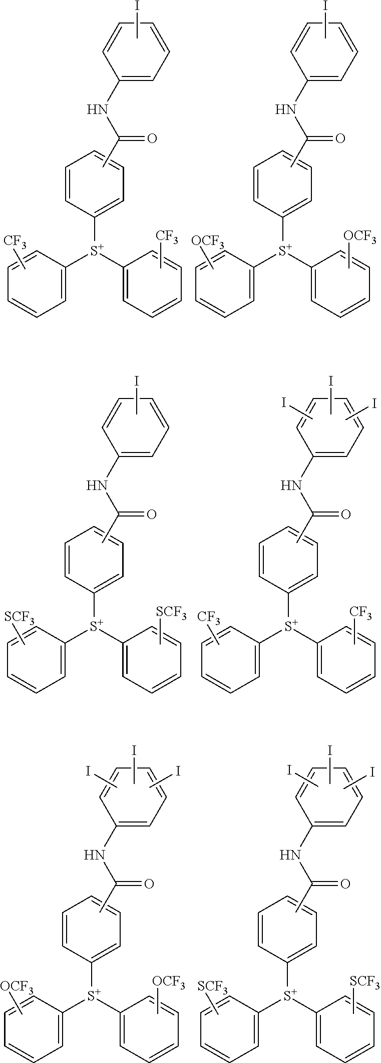

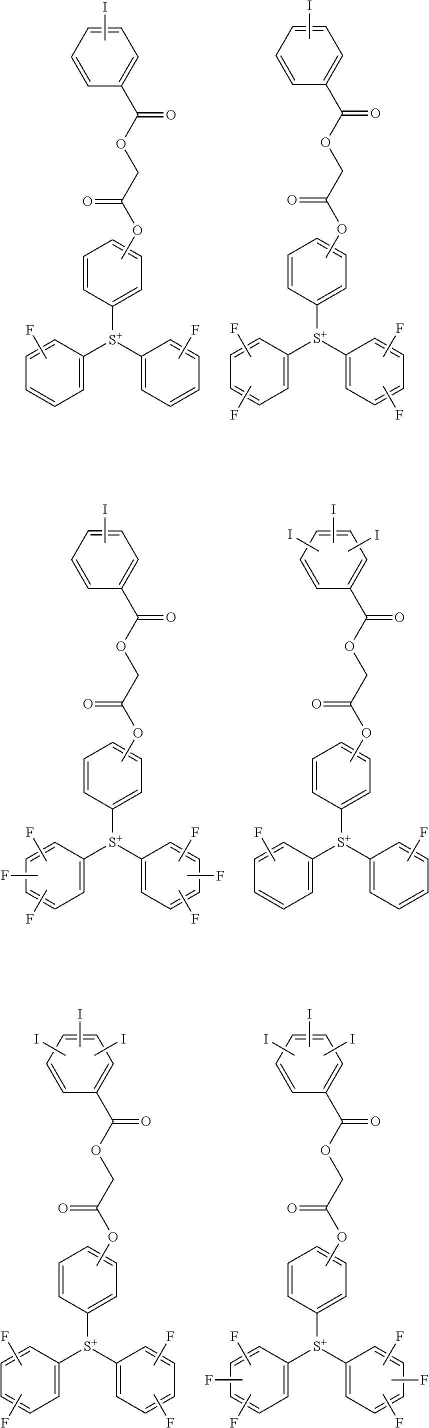

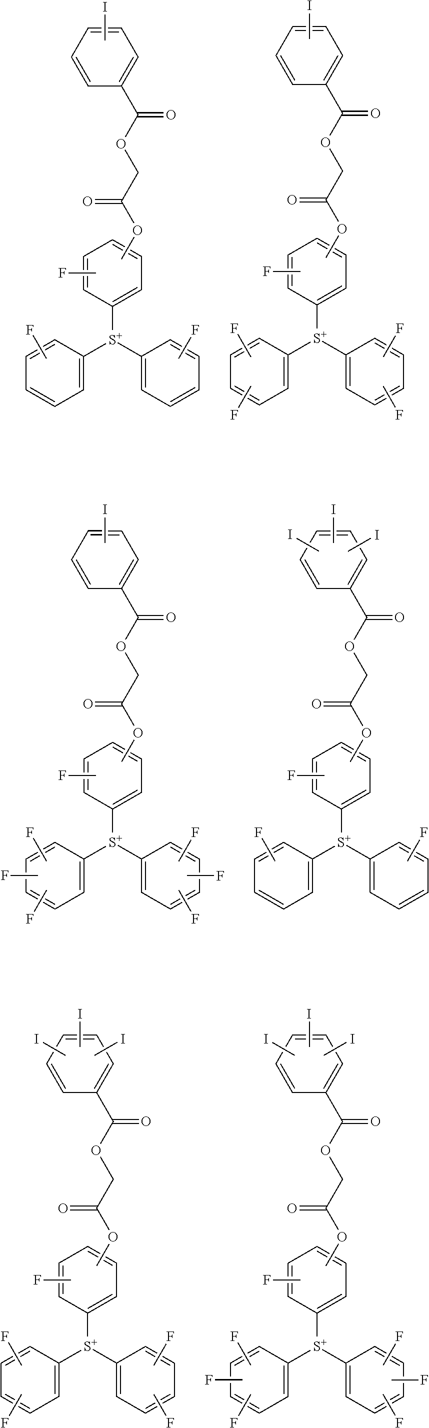

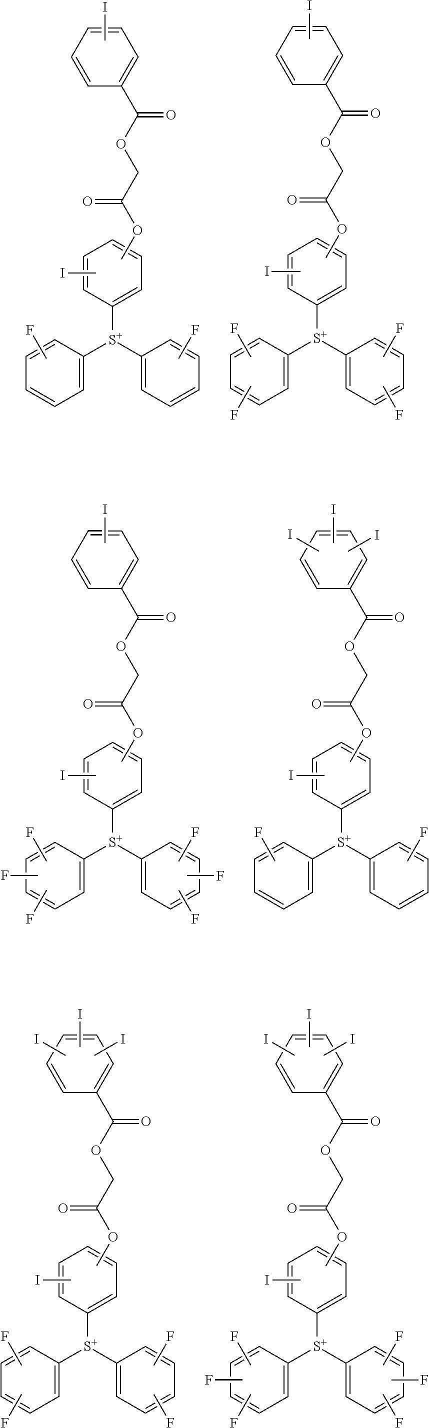

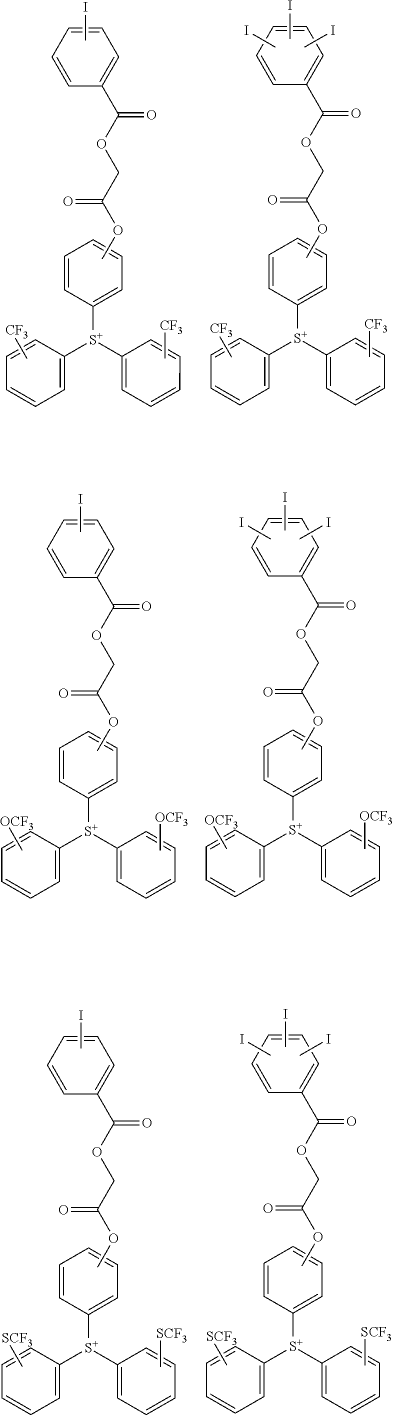

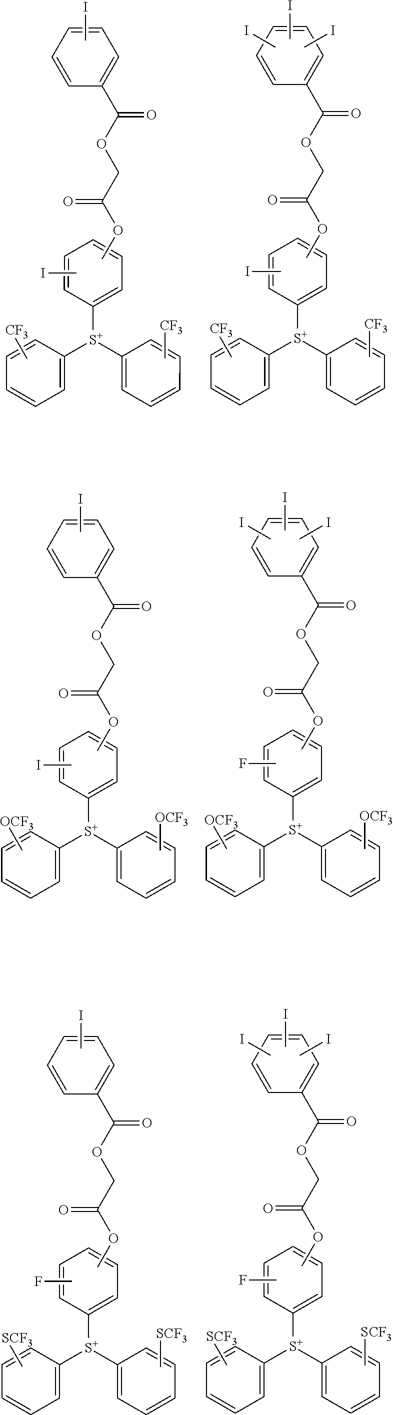

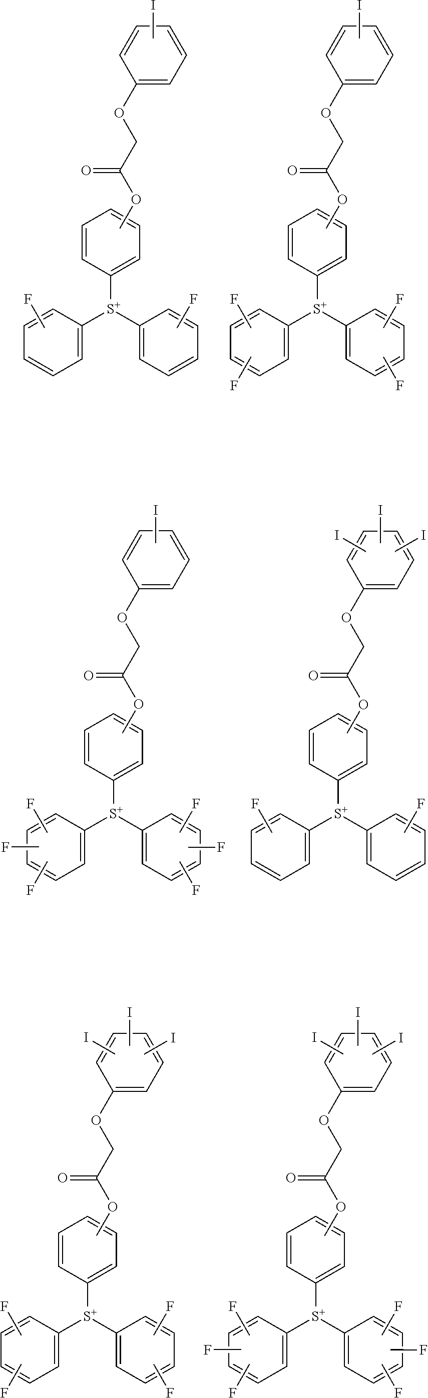

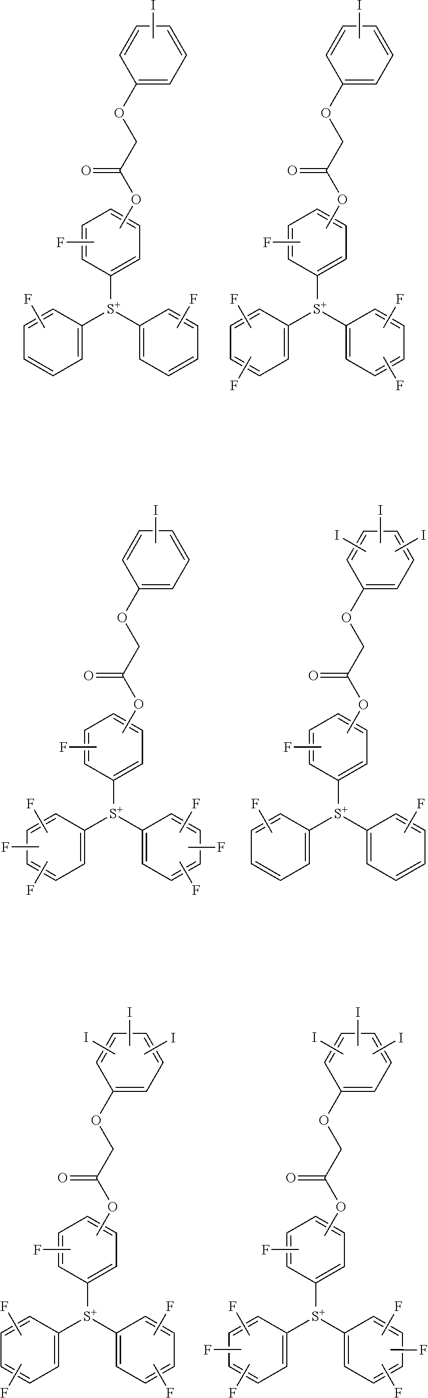

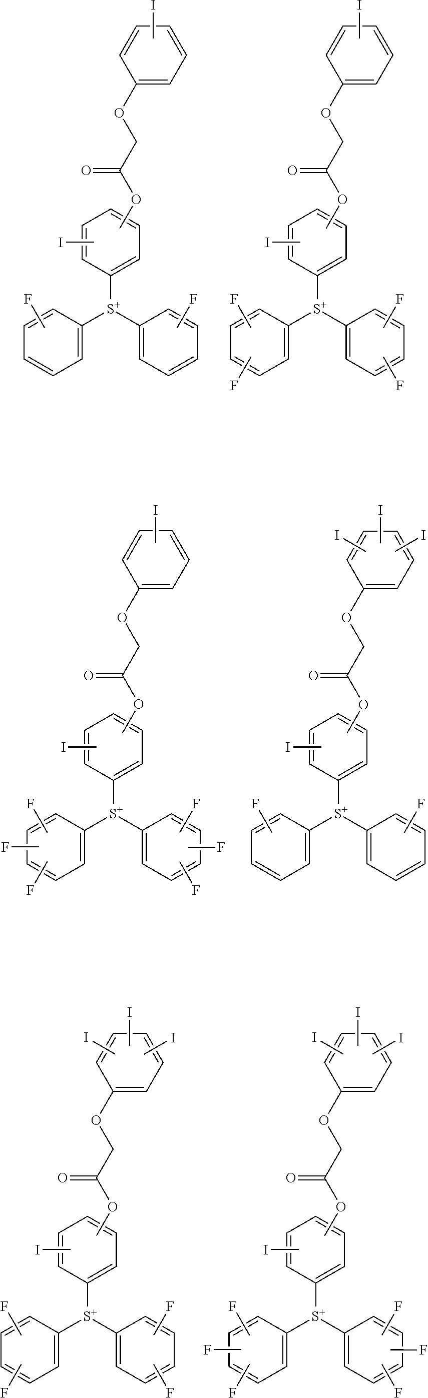

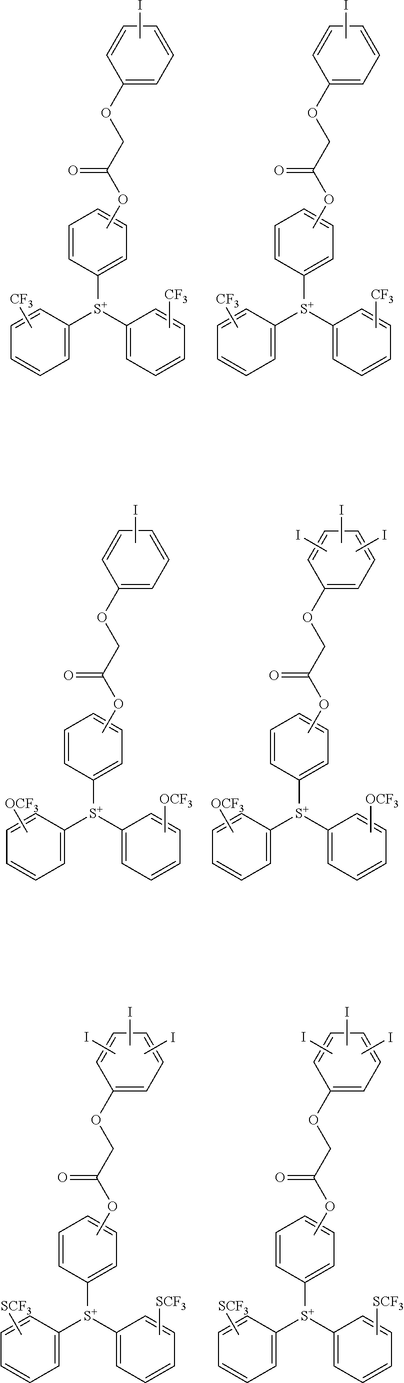

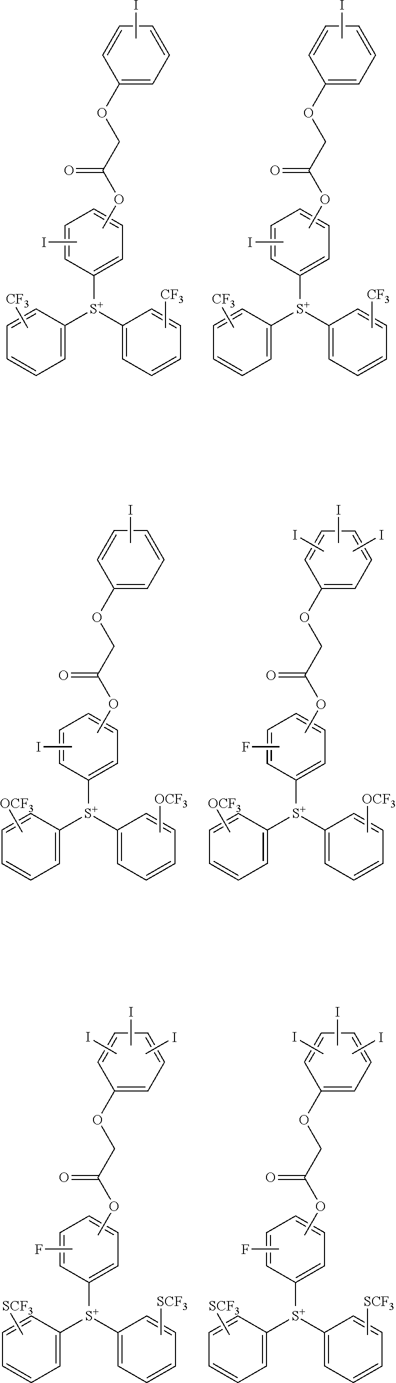

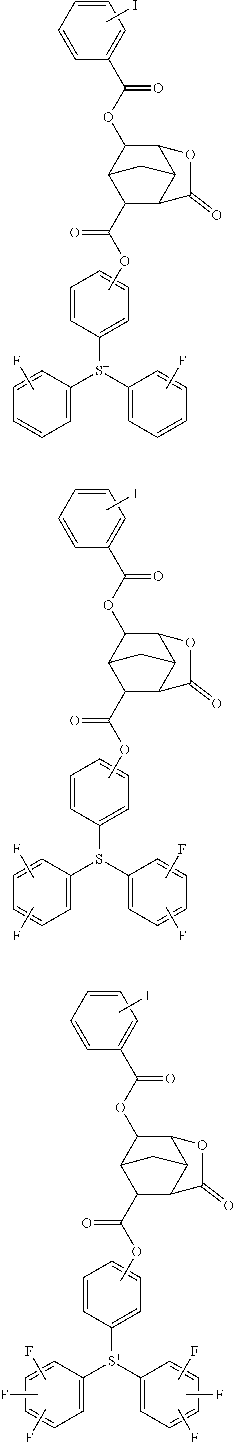

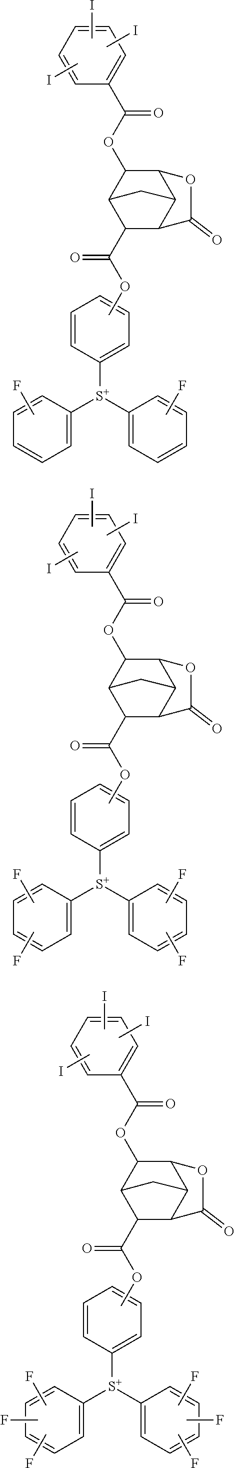

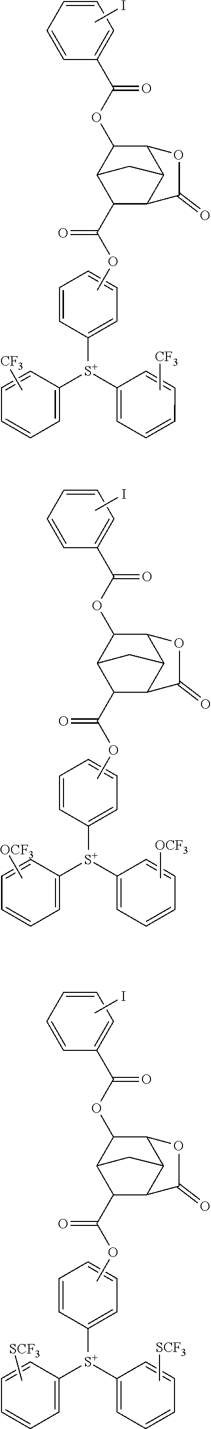

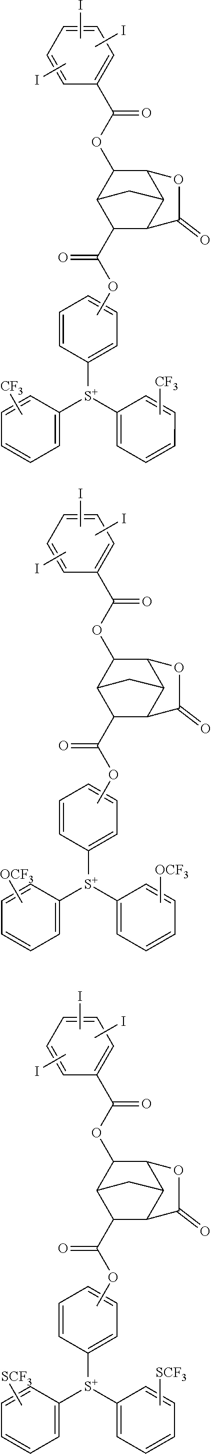

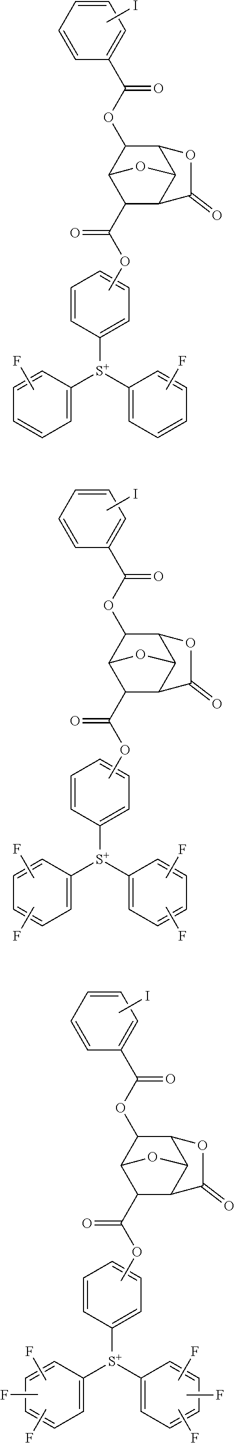

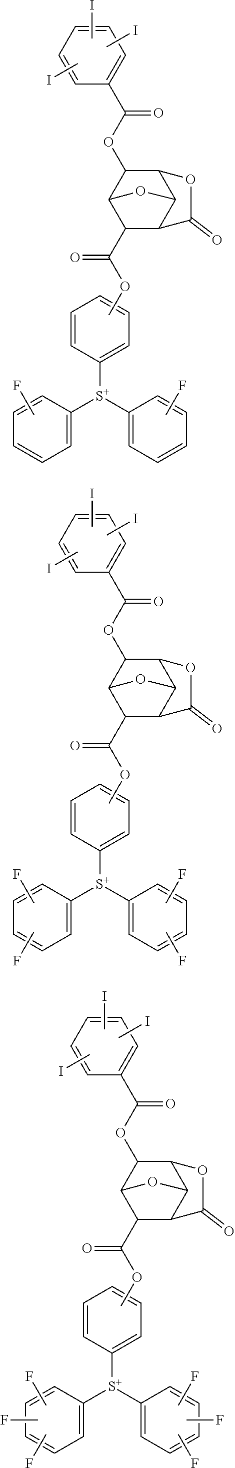

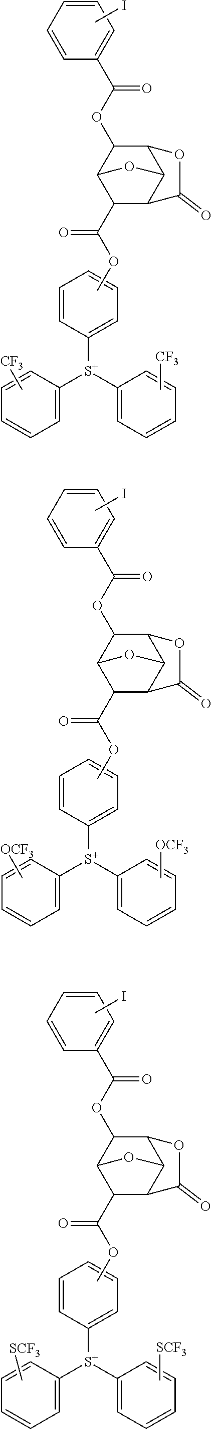

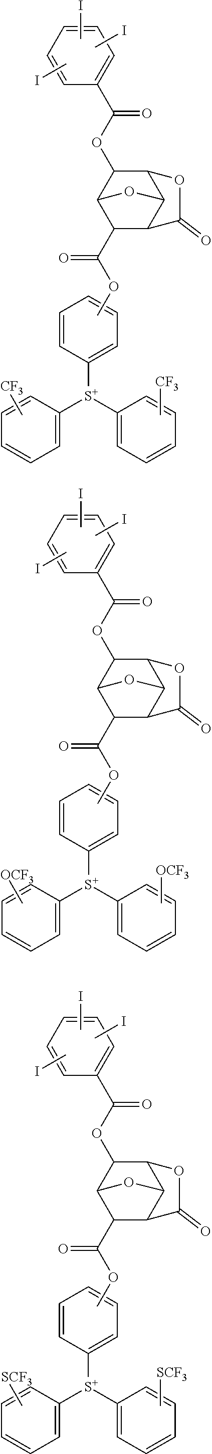

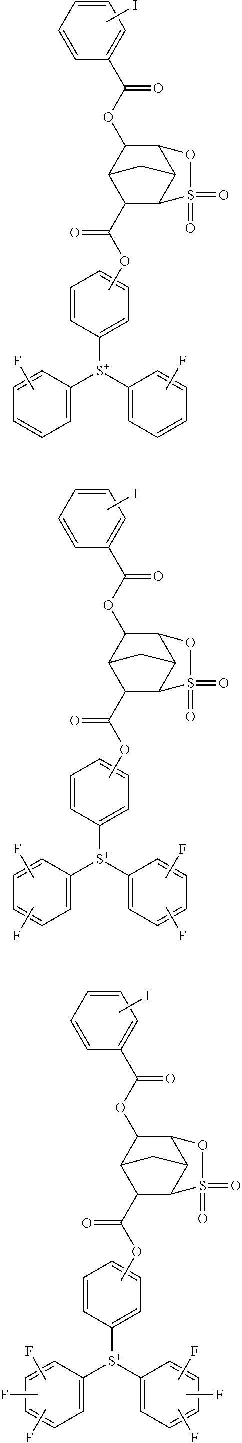

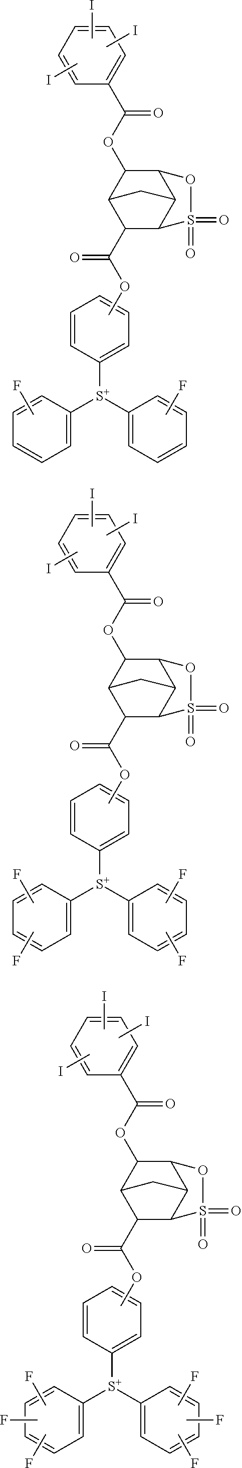

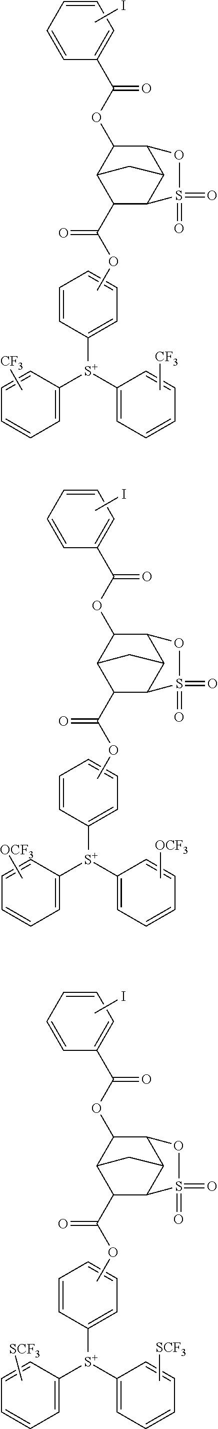

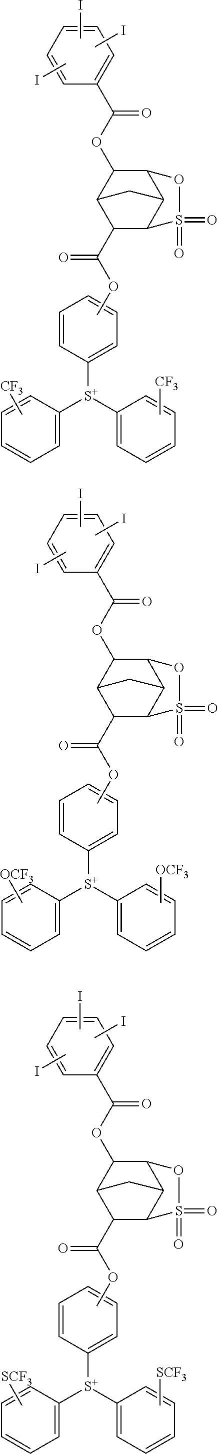

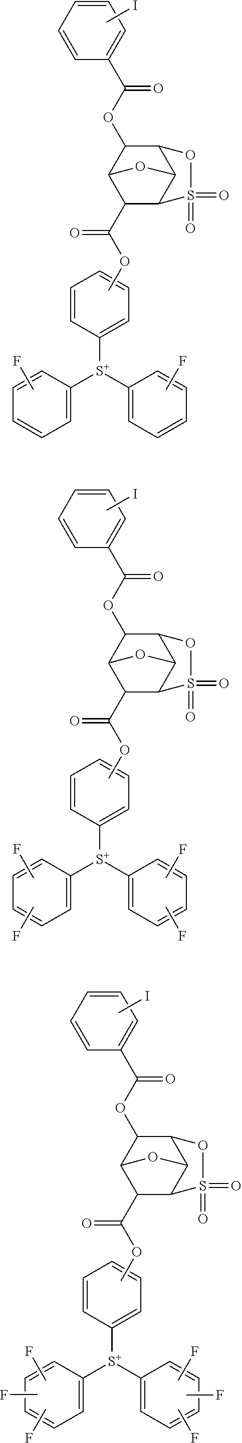

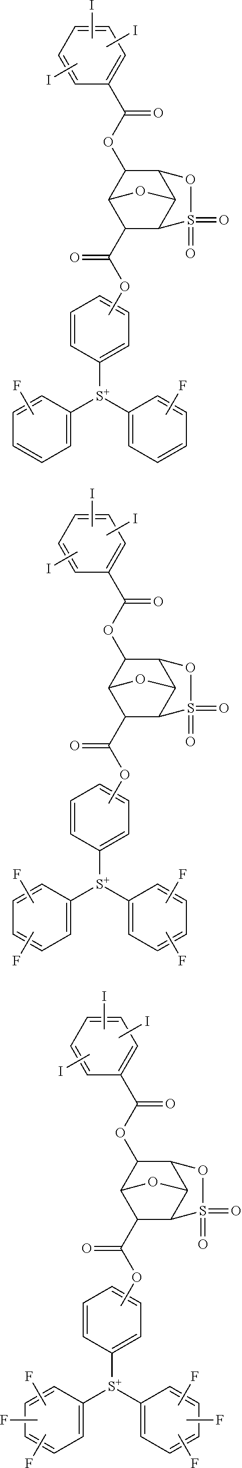

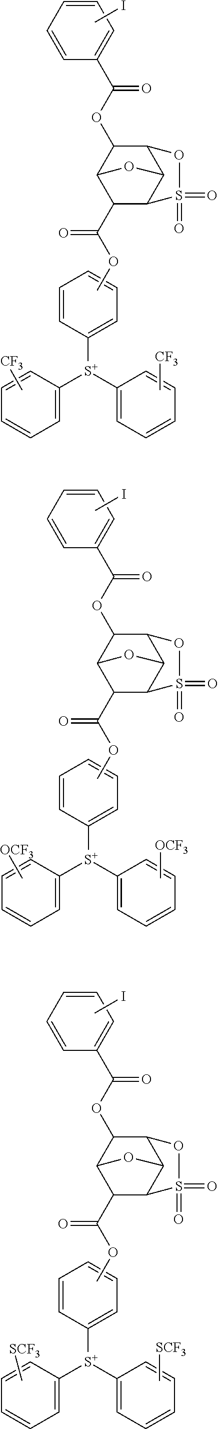

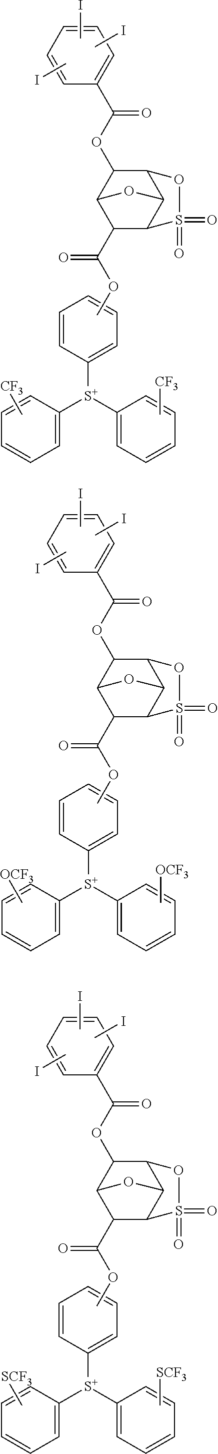

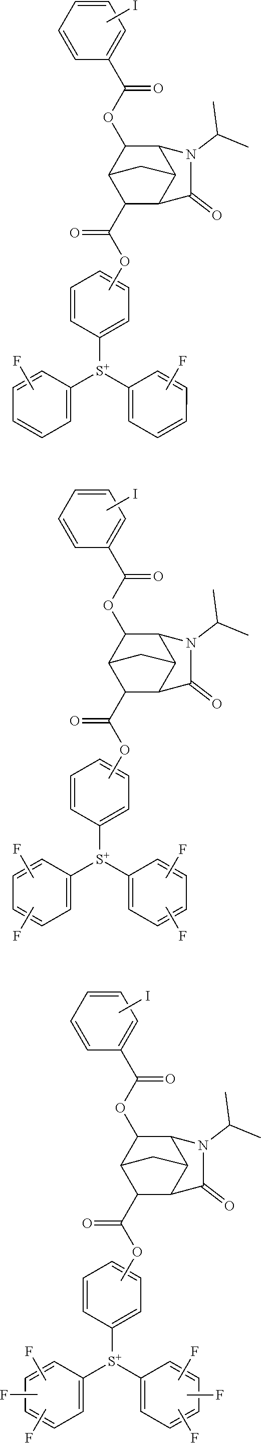

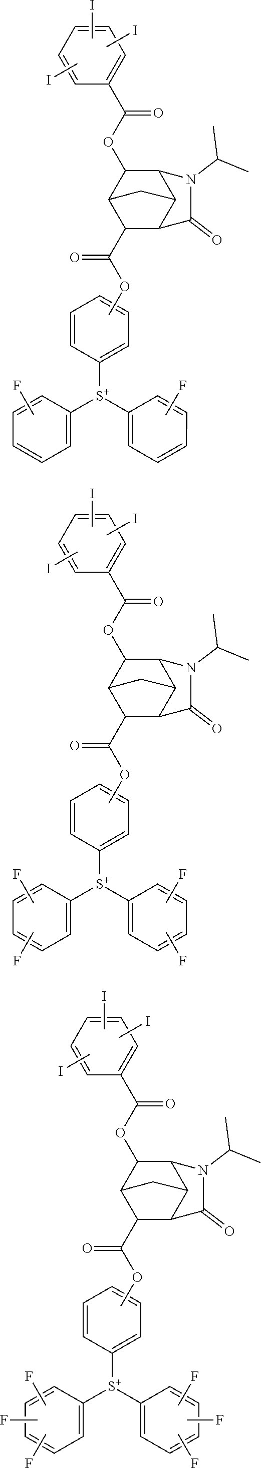

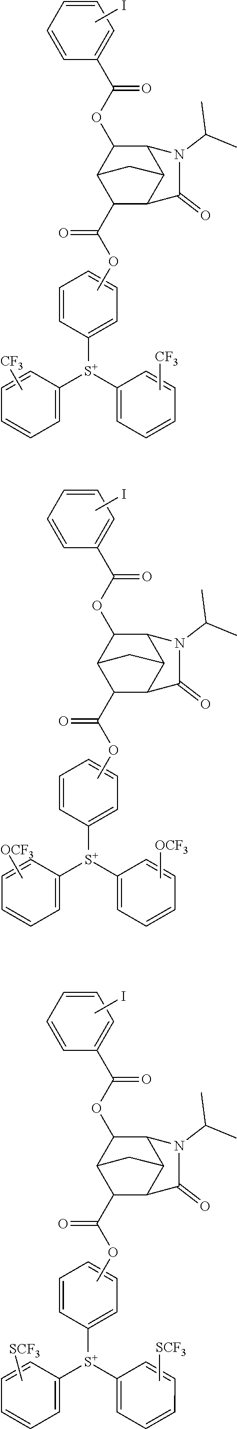

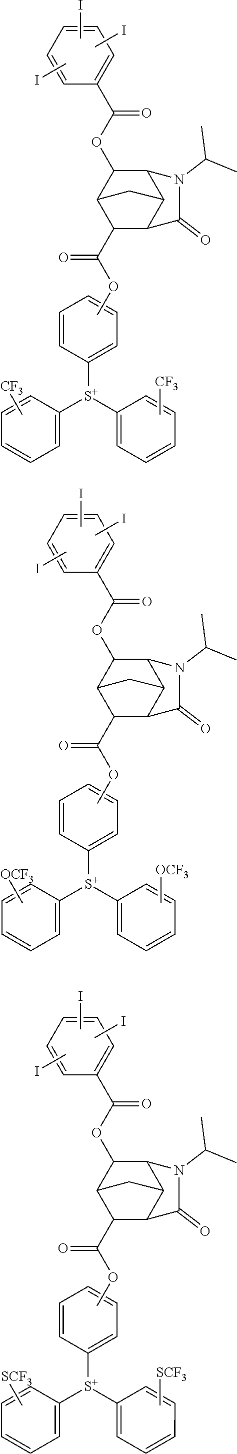

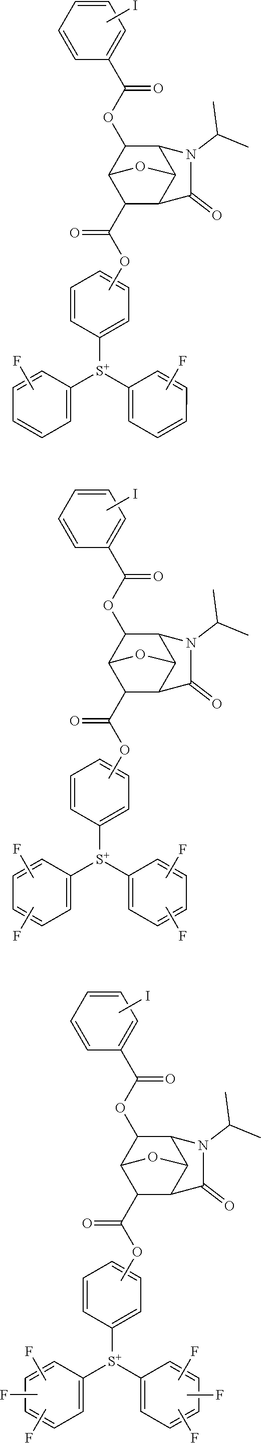

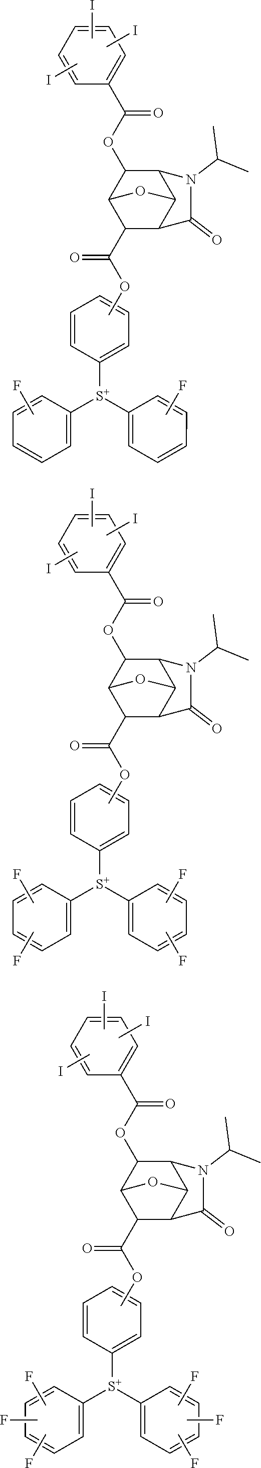

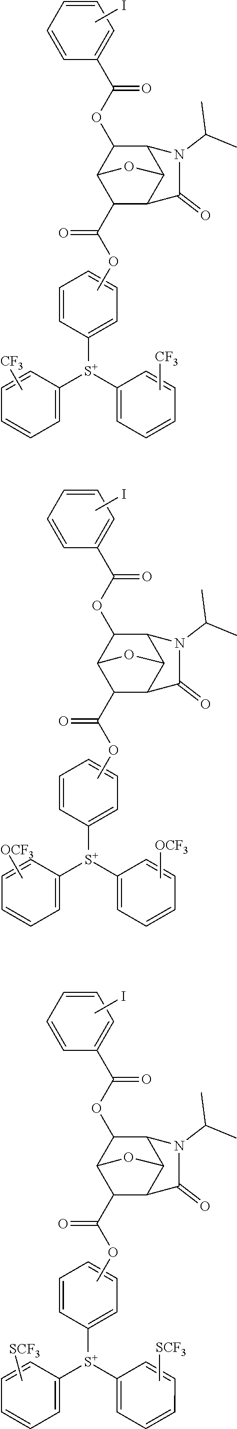

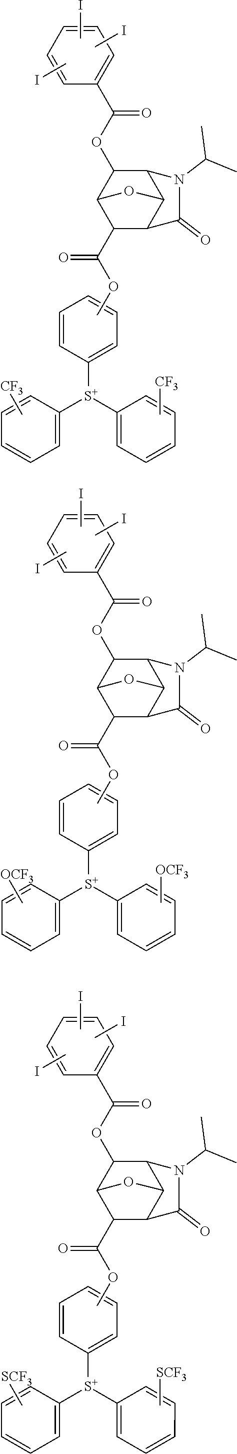

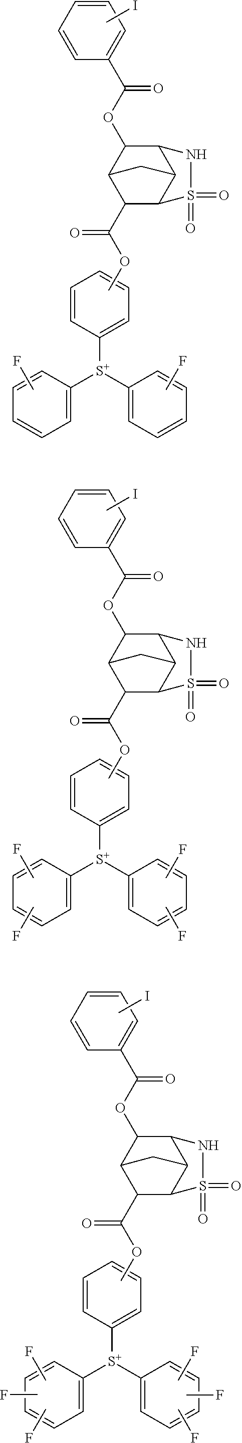

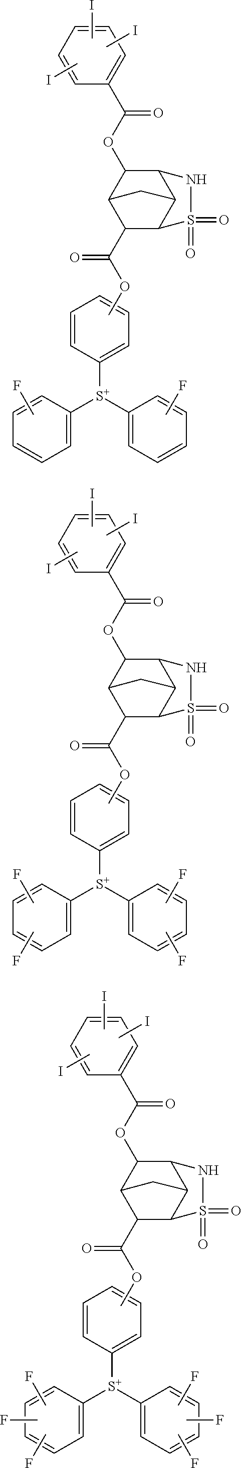

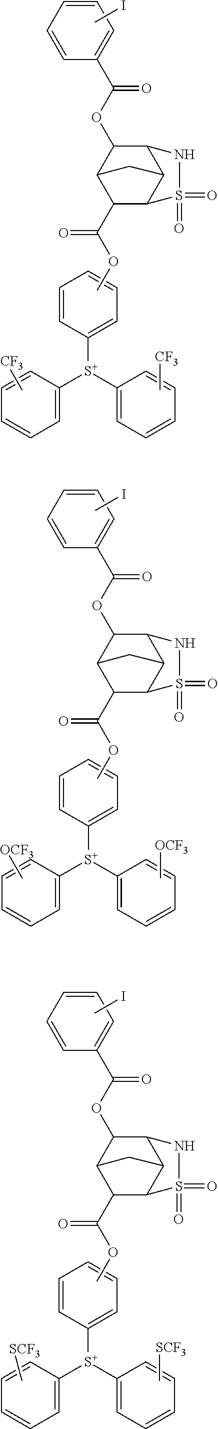

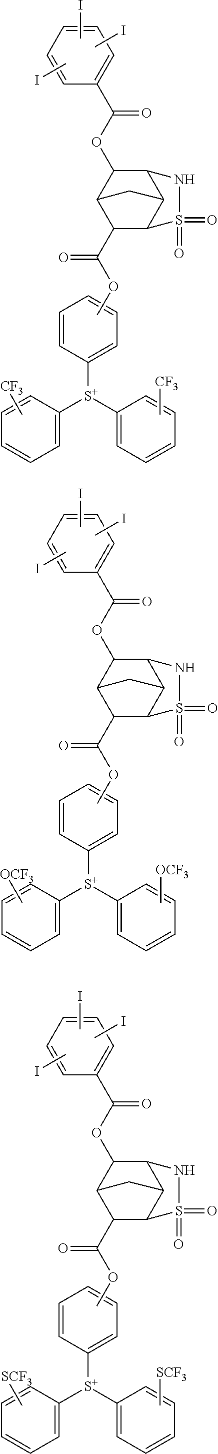

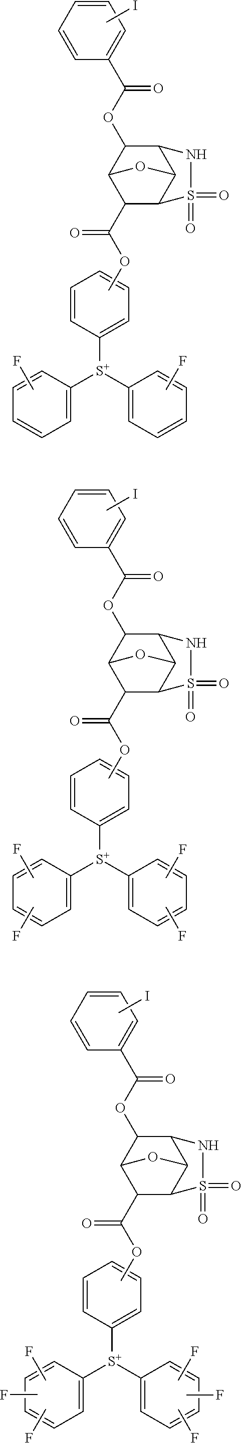

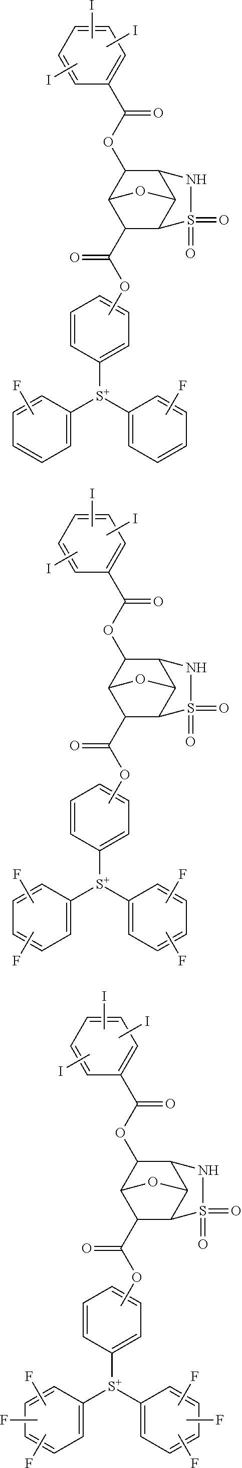

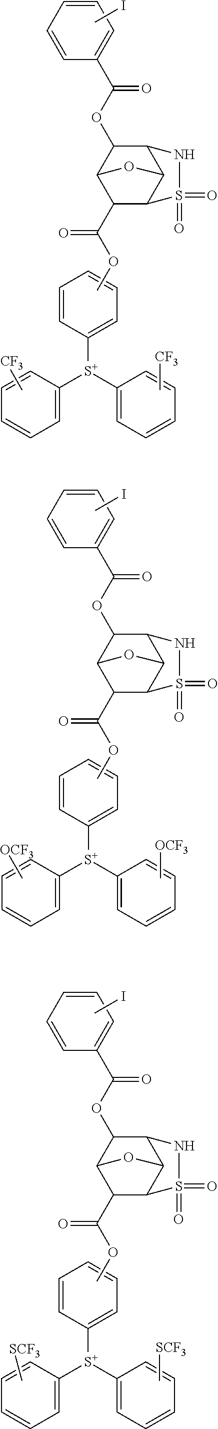

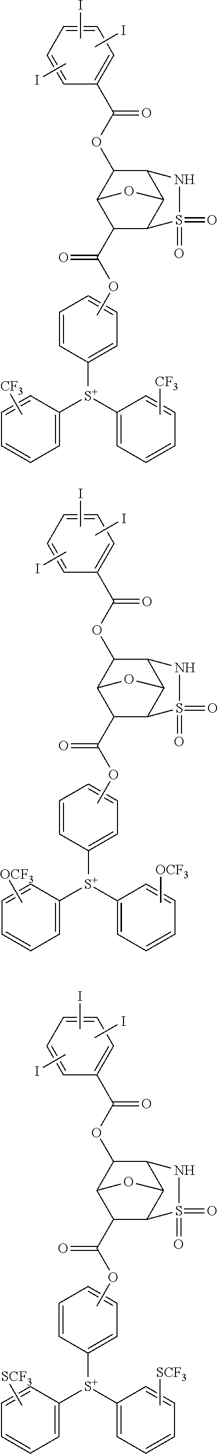

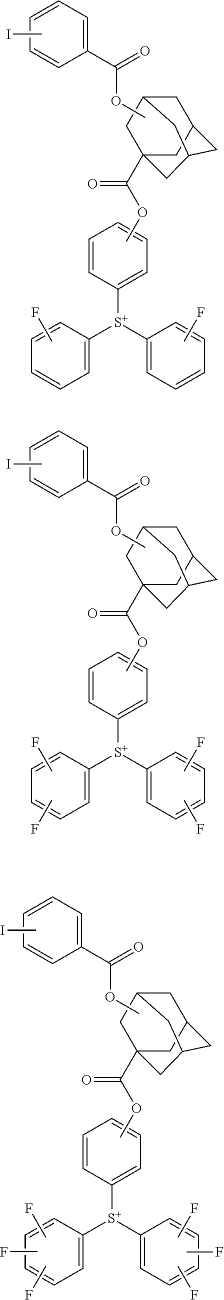

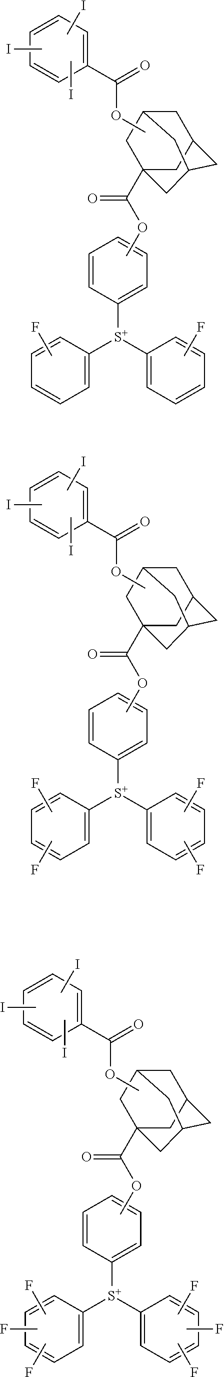

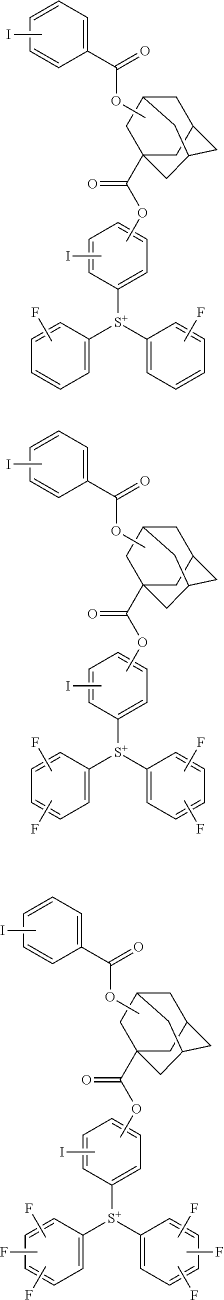

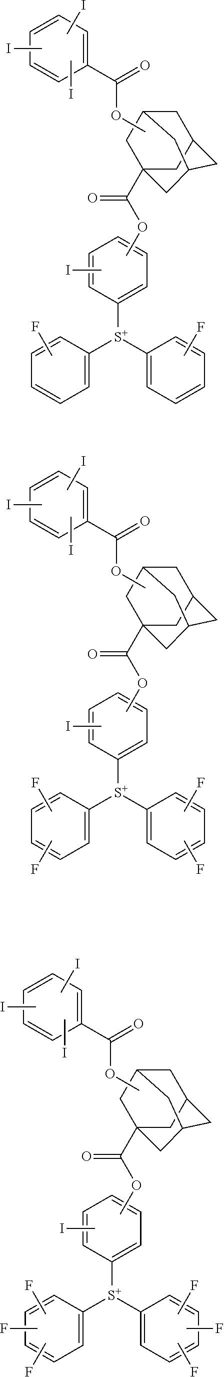

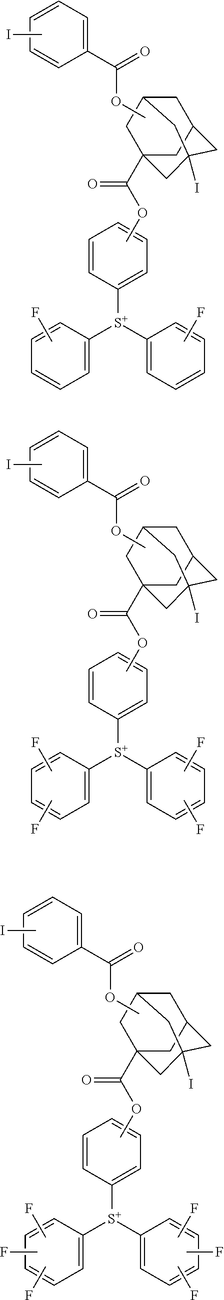

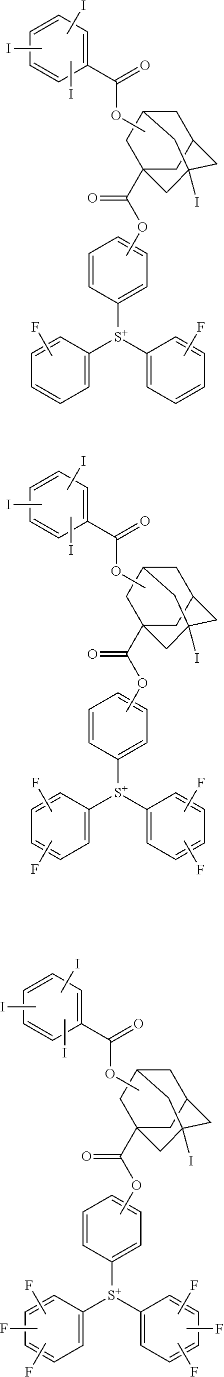

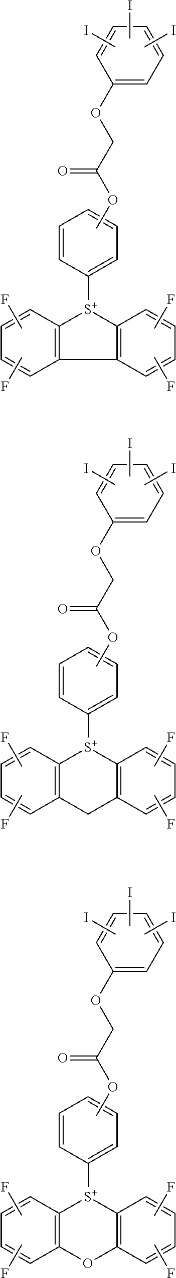

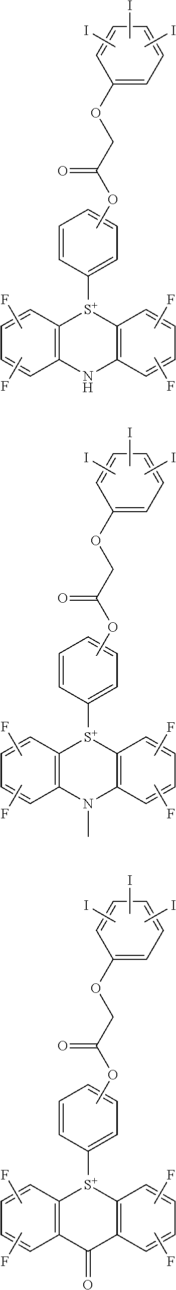

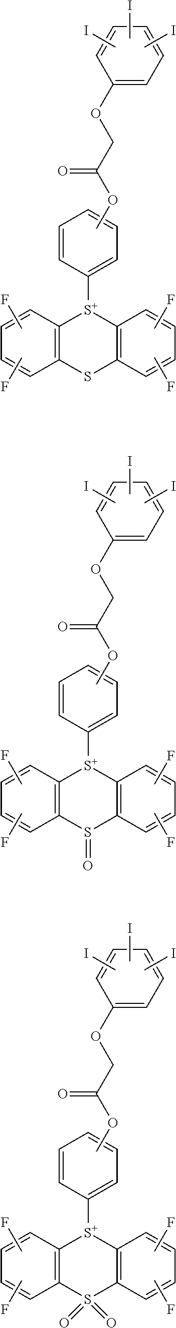

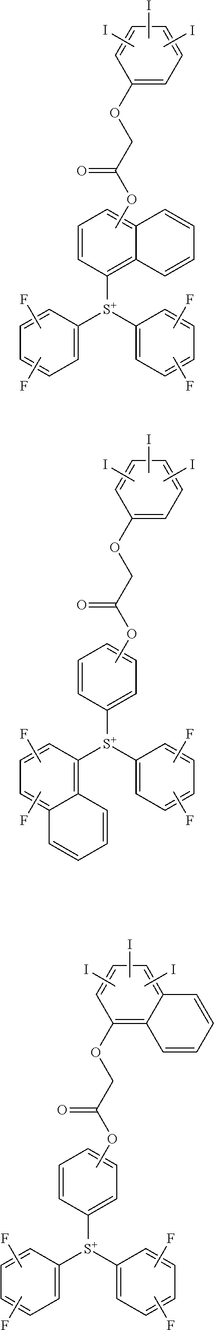

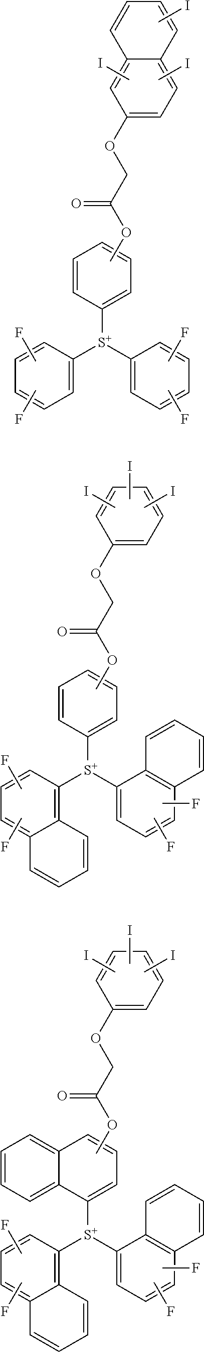

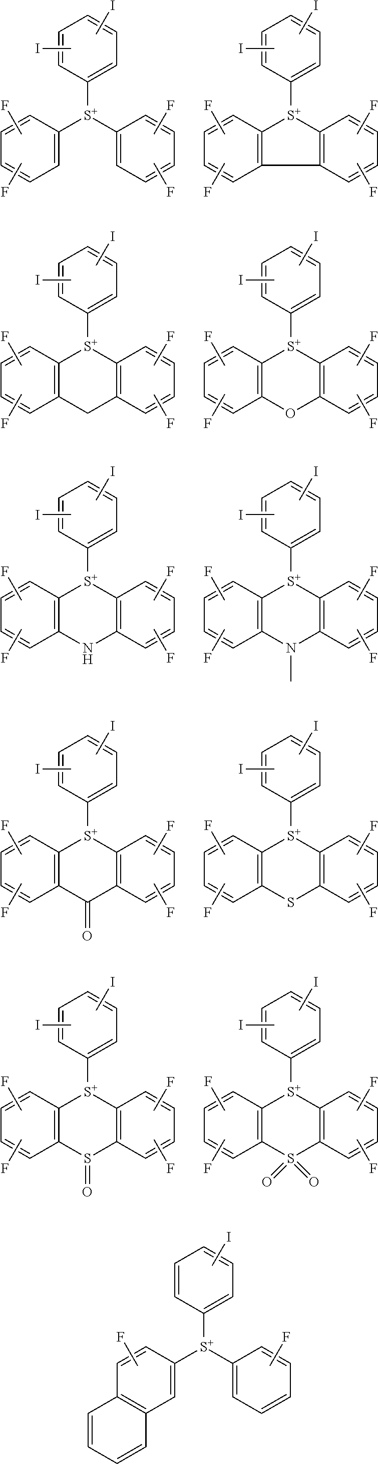

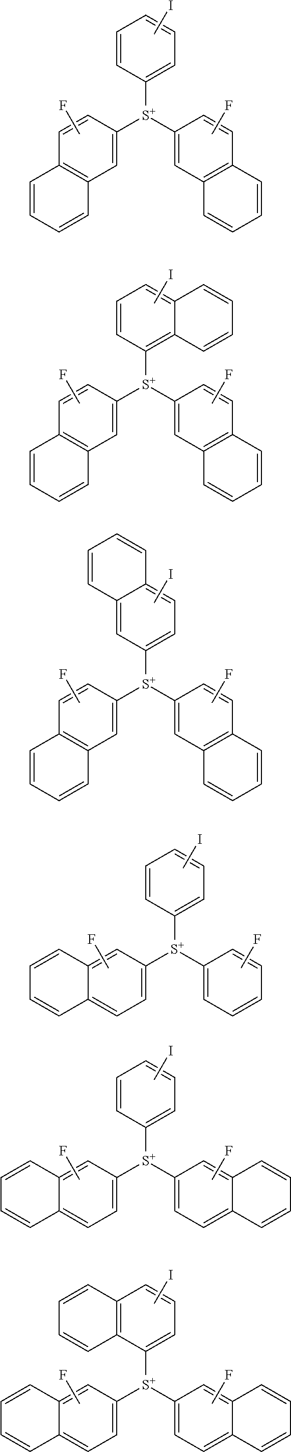

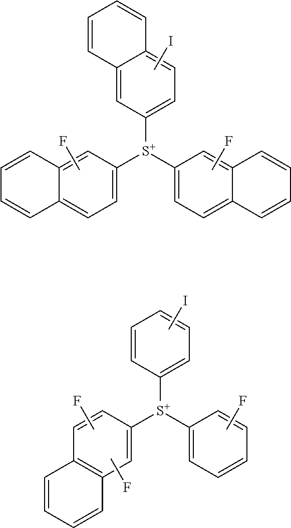

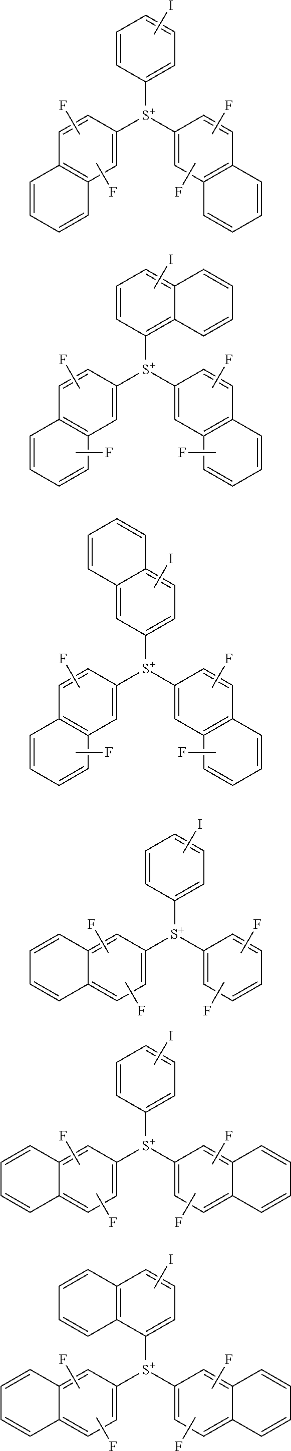

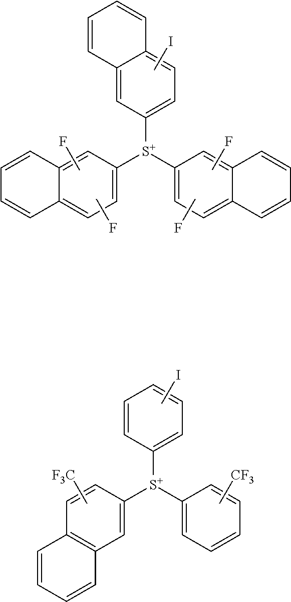

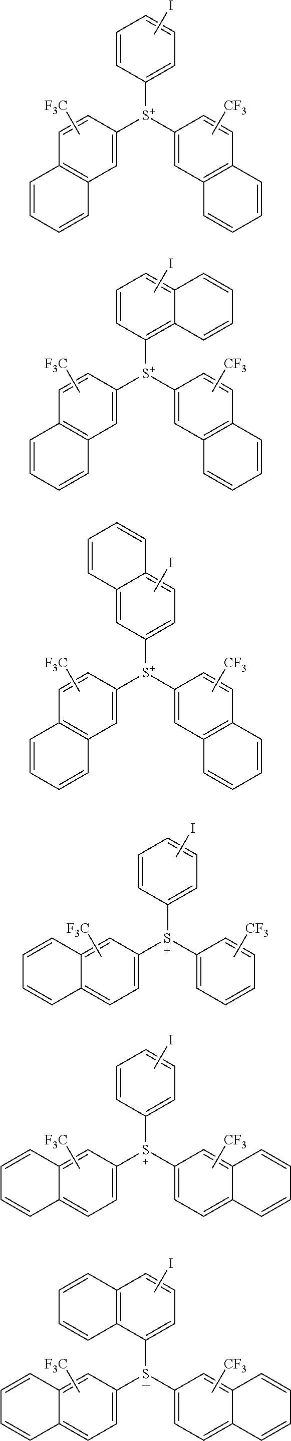

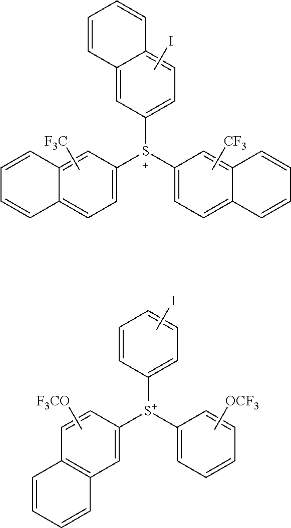

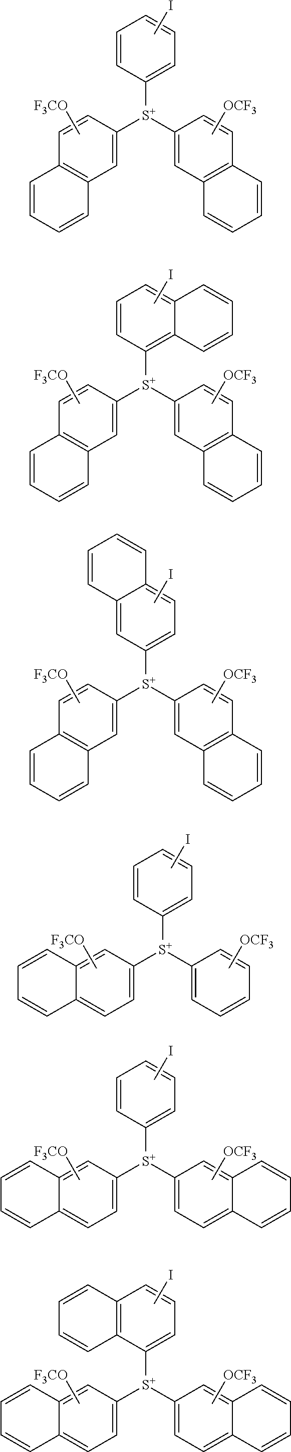

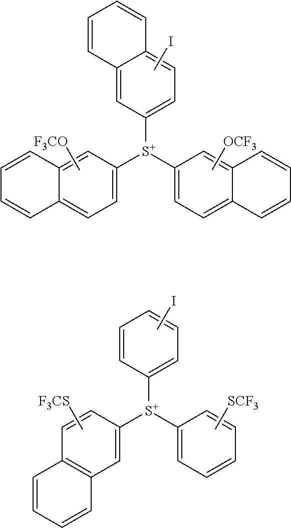

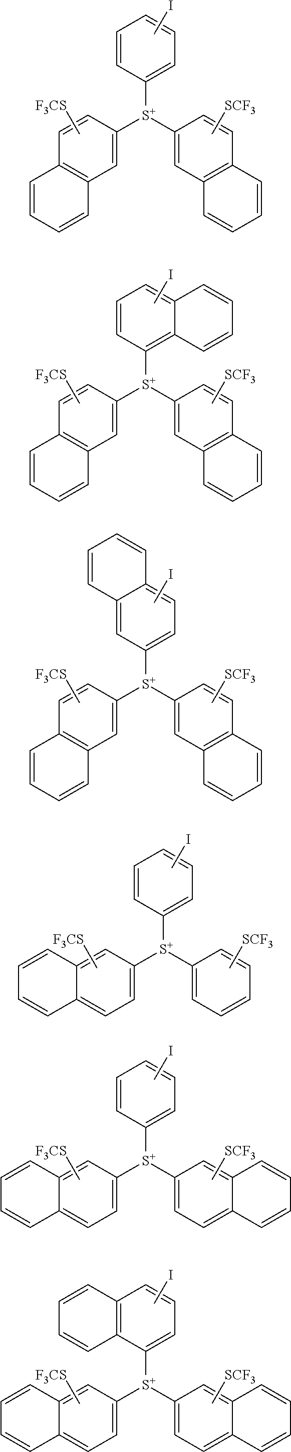

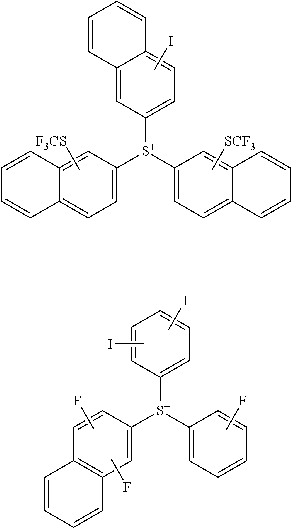

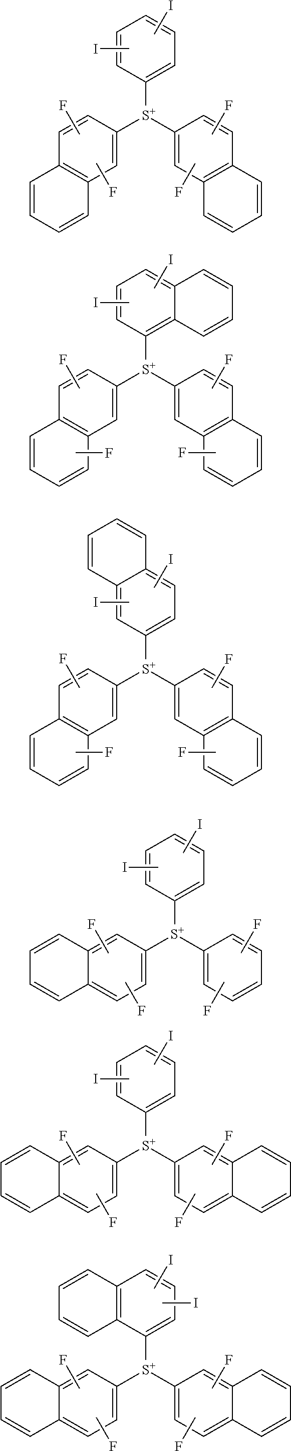

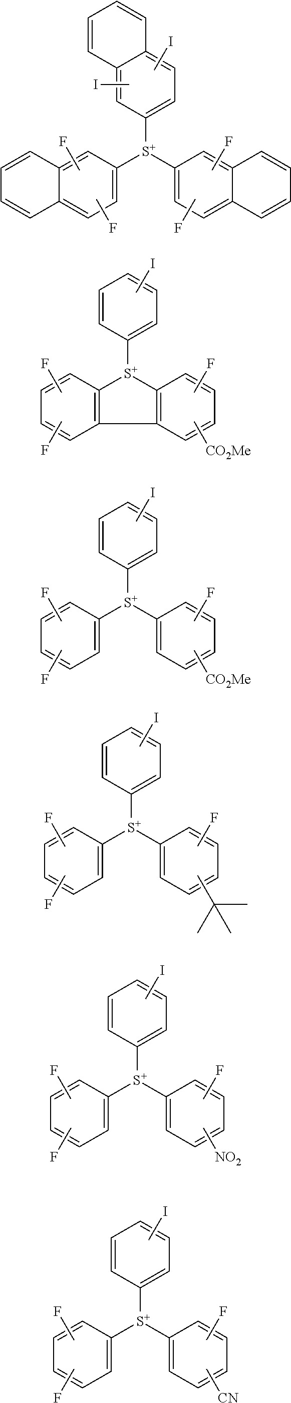

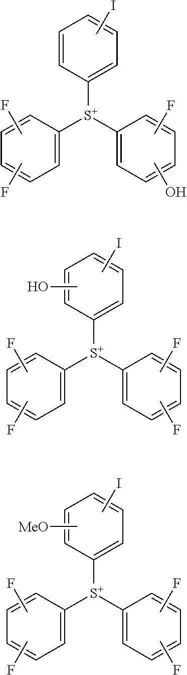

Specific examples of the onium salt of the present invention include any combination of the above-described anions and cations.

The inventive onium-salt-type monomer can be synthesized by a known method. Specifically, in a case where an acetal-type acid-labile group is introduced into the following intermediate (In-1-ex), which can be synthesized by using a known organic synthesis reaction, the group can be introduced by reacting a corresponding chloromethyl alkyl ether under conditions of basicity. Meanwhile, by reacting an alkyl chloroformate derived from a tertiary alcohol under conditions of basicity, a carbonate-ester-type acid-labile group can be introduced.

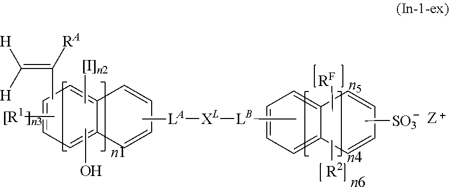

In the formula, “n1” to “n6”, R^{A}, R¹, R², R^{F}, L^{A}, L^{B}, X^{L}, and Z⁺ are as defined above.

Incidentally, the above-described manufacturing method is only an example, and methods for manufacturing the inventive onium-salt-type monomer are not limited thereto.

Furthermore, it is possible to use the inventive onium-salt-type monomer itself as a monomeric photo-acid generator.

### [Polymer]

The inventive polymer contains a repeating unit which is a derivative of the onium-salt-type monomer represented by the formula (a) (hereinafter, also referred to as repeating unit-a).

The inventive polymer is a polymer-bound photo-acid generator that functions as a base polymer in a chemically-amplified resist composition, and also functions as a photo-acid generator. A structural characteristic of the inventive onium-salt-type monomer is that the monomer has an acid-labile group in the anion, and by thus having an increased number of carbon atoms, the monomer has favorable solvent solubility. The acid-labile group in the anion causes a deprotection reaction in association with the generation of an acid, and generates an aromatic hydroxy group. Thus, dissolution contrast can be imparted. Furthermore, an iodine atom in the anion very greatly absorbs EUV in EUV lithography at a wavelength of 13.5 nm, and therefore, secondary electrons are generated from the iodine atom during exposure. By the anion structure being bonded to the aromatic ring substituted with the polymerizable group and the iodine atom, secondary electrons generated from the iodine atom promote the decomposition of the cation located near the anion, and an acid is generated efficiently. Thus, higher sensitivity is achieved. Furthermore, the atomic weight of an iodine atom is high and the polymer has a structure where the generated acid is bonded to the polymer main chain, and therefore, the polymer has a characteristic that acid diffusion is low. A polymerizable group including a styrene or vinylnaphthalene structure is more rigid than a polymerizable group of a methacrylate ester or the like, and increases the glass transition temperature (Tg) of the polymer. It can be considered that, by interaction between aromatic rings within a base polymer or aromatic rings of base polymers (n-n stacking effect), base polymers are arranged regularly, and resistance to pattern collapse against a developer is exhibited at the time of fine pattern formation. In addition, in an etching process after the fine pattern formation, excellent etching resistance is exhibited by the polymer having an aromatic ring directly bonded to the main chain. As the cation, a triarylsulfonium cation containing a fluorine atom is preferably contained, and by the electron-withdrawing effect of the fluorine atom, the energy level of the LUMO in the frontier orbital theory is lowered and the generated secondary electrons can be more easily received. Thus, the decomposition of the cation is promoted, and an acid is generated effectively. By the combined effect of the above, higher sensitivity is achieved. Thus, it is possible to prevent degradation in resolution caused by blurring due to acid diffusion, and LWR and CDU can be improved. Accordingly, the inventive polymer is particularly suitable as a material of a chemically-amplified positive resist composition.

The polymer may further include a repeating unit represented by the following formula (b1) (hereinafter, also referred to as repeating unit-b1) or a repeating unit represented by the following formula (b2) (hereinafter, also referred to as repeating unit-b2).

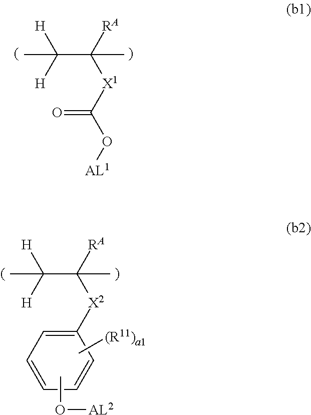

In the formulae (b1) and (b2), each R^{A} independently represents a hydrogen atom, a fluorine atom, a methyl group, or a trifluoromethyl group.

In the formula (b1), X¹ represents a single bond, a phenylene group, a naphthylene group, *—C(═O)—O—X¹¹—, or *—C(═O)—NH—X¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, and X¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring. “*” represents an attachment point to the carbon atom of the main chain.

In the formula (b2), X² represents a single bond, *—C(═O)—O—, or *—C(═O)—NH—. “*” represents an attachment point to the carbon atom of the main chain. R¹¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. “a1” represents 0, 1, 2, 3, or 4, preferably 0 or 1.

In the formulae (b1) and (b2), AL¹ and AL² each independently represent an acid-labile group. Specific examples of the acid-labile groups include those disclosed in JP 2013-80033 A and JP 2013-83821 A, but are not limited thereto.

Typically, specific examples of the acid-labile groups include those represented by the following formulae (AL-1) to (AL-3).

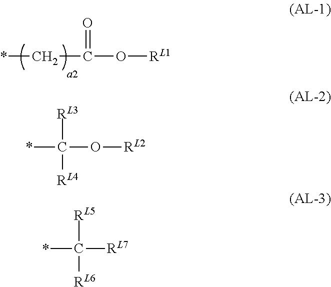

In the formulae, “*” represents an attachment point.

In the formulae (AL-1) and (AL-2), R^{L1} and R^{L2} each independently represent a hydrocarbyl group having 1 to 40 carbon atoms and optionally containing a heteroatom such as an oxygen atom, a sulfur atom, a nitrogen atom, a fluorine atom, or an iodine atom. The hydrocarbyl groups may be saturated or unsaturated, and may be linear, branched, or cyclic. As the hydrocarbyl groups, ones having 1 to 20 carbon atoms are preferable.

In the formula (AL-1), “a2” represents an integer of 0 to 10, preferably 1, 2, 3, 4, or 5.

In the formula (AL-2), R^{L3} and R^{L4} each independently represent a hydrogen atom or a hydrocarbyl group having 1 to 20 carbon atoms and optionally containing a heteroatom such as an oxygen atom, a sulfur atom, a nitrogen atom, a fluorine atom, or an iodine atom. The hydrocarbyl group may be saturated or unsaturated, and may be linear, branched, or cyclic.

Furthermore, any two of R^{L2}, R^{L3}, and R^{L4} may be bonded to each other to form a ring having 3 to 20 carbon atoms together with the carbon atom or the carbon atom and the oxygen atom bonded thereto. As the ring, a ring having 4 to 16 carbon atoms is preferable, and an aliphatic ring is particularly preferable.

In the formula (AL-3), R^{L5}, R^{L6}, and R^{L7} each independently represent a hydrocarbyl group having 1 to 20 carbon atoms, and may contain a heteroatom such as an oxygen atom, a sulfur atom, a nitrogen atom, a fluorine atom, or an iodine atom. The hydrocarbyl group may be saturated or unsaturated, and may be linear, branched, or cyclic. Furthermore, any two of R^{L5}, R^{L6}, and R^{L7} may be bonded to each other to form a ring having 3 to 20 carbon atoms together with the carbon atom bonded thereto. As the ring, a ring having 4 to 16 carbon atoms is preferable, and an aliphatic ring is particularly preferable.

Specific examples of the repeating unit-b1 include the following, but are not limited thereto. Note that, in the following formulae, R^{A} and AL¹ are as defined above.

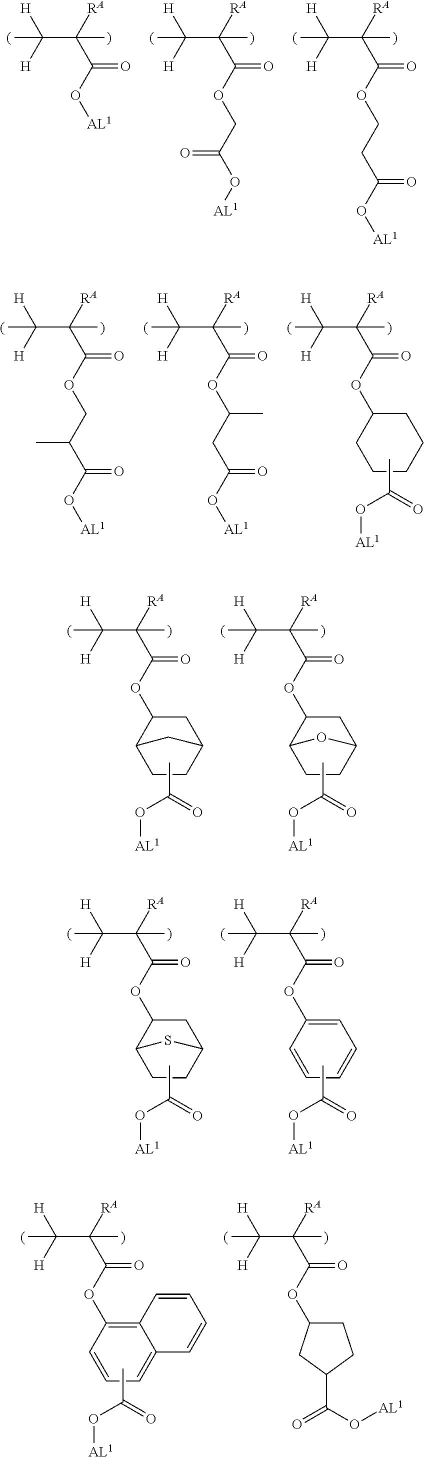

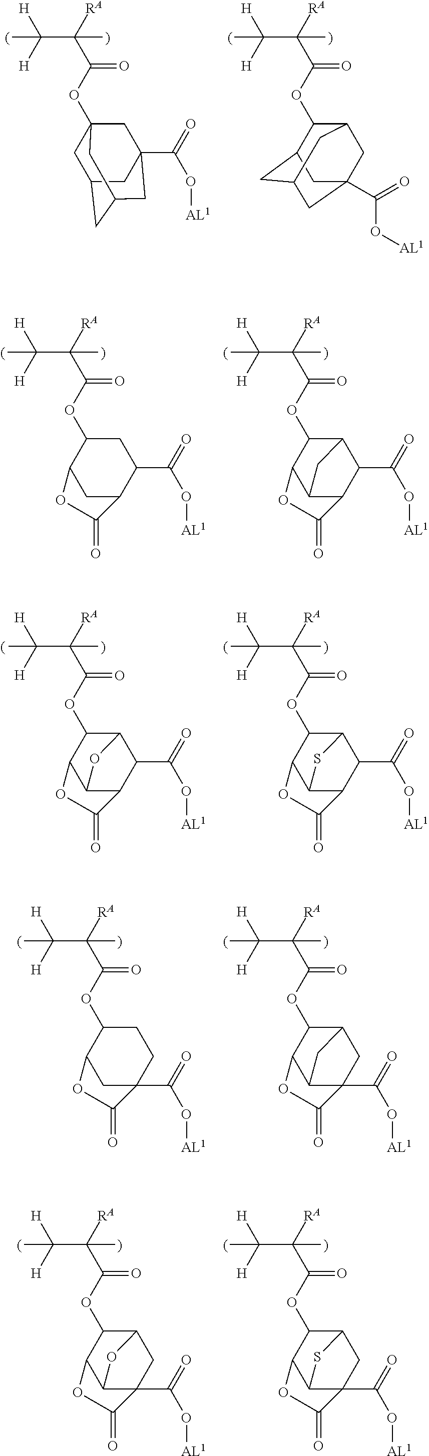

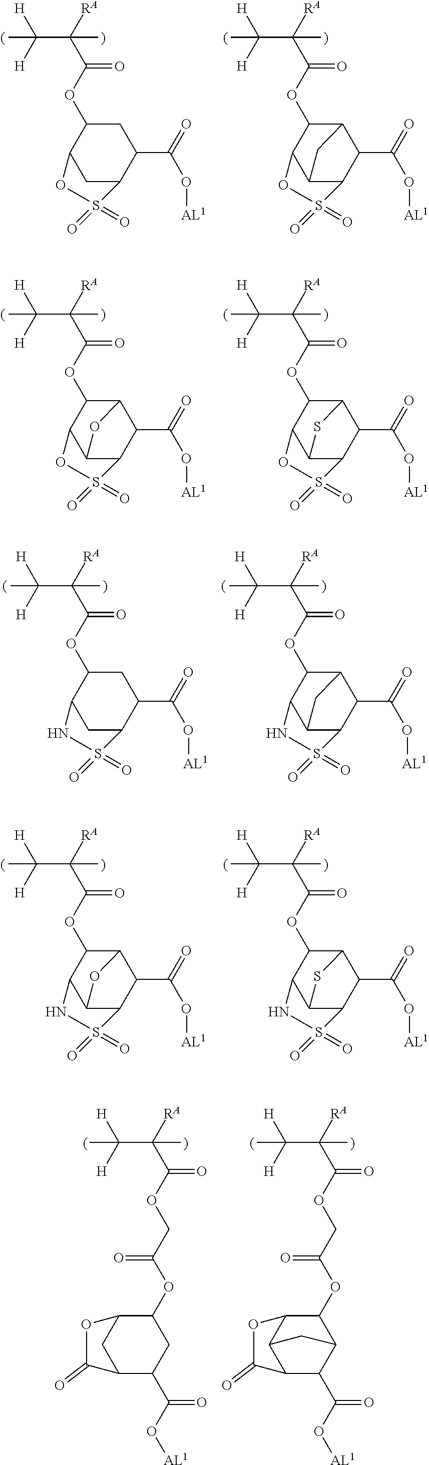

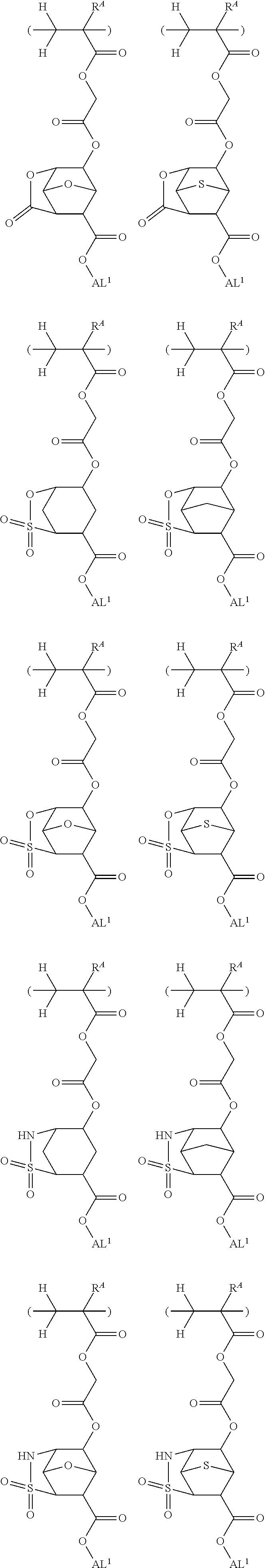

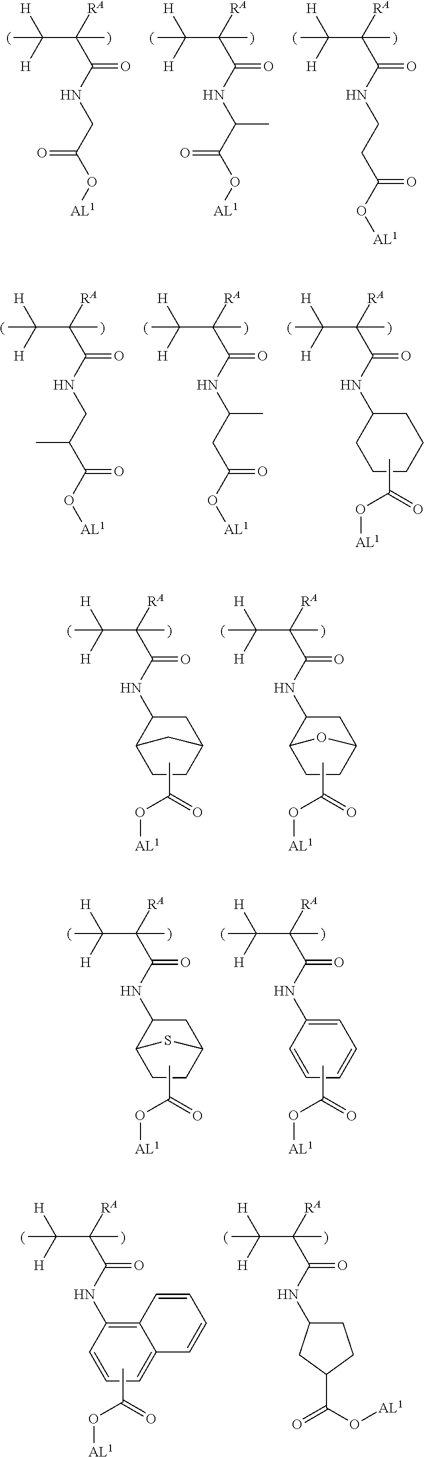

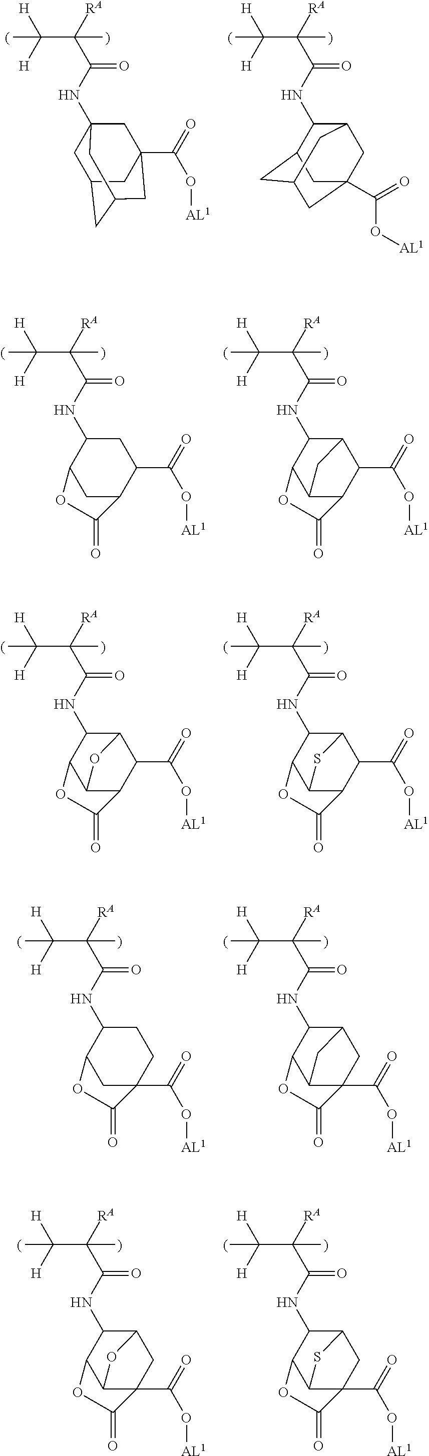

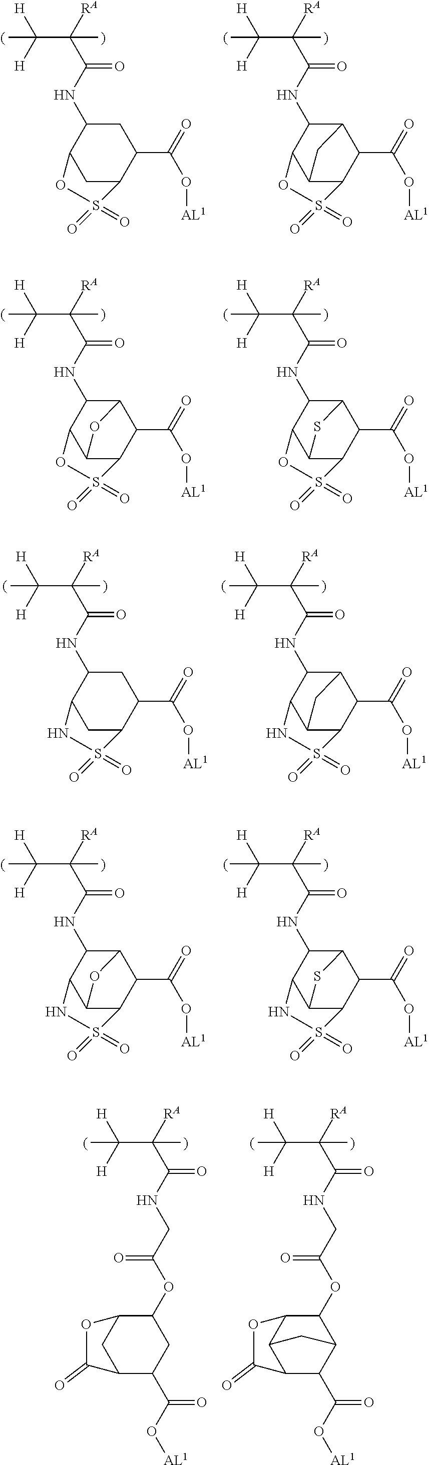

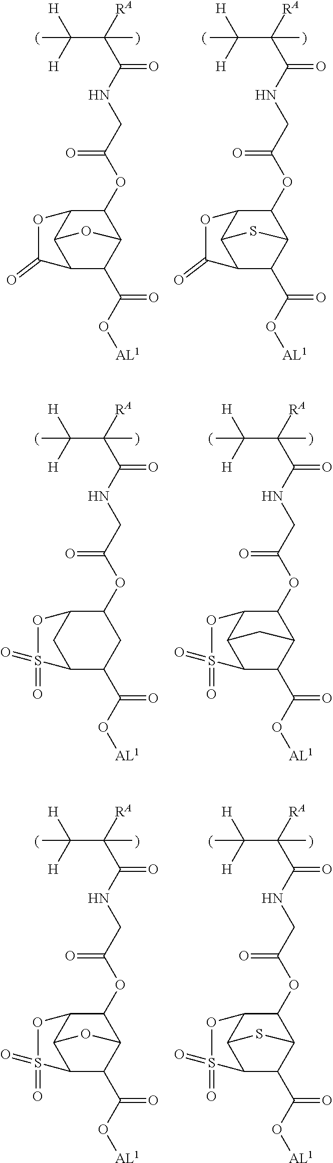

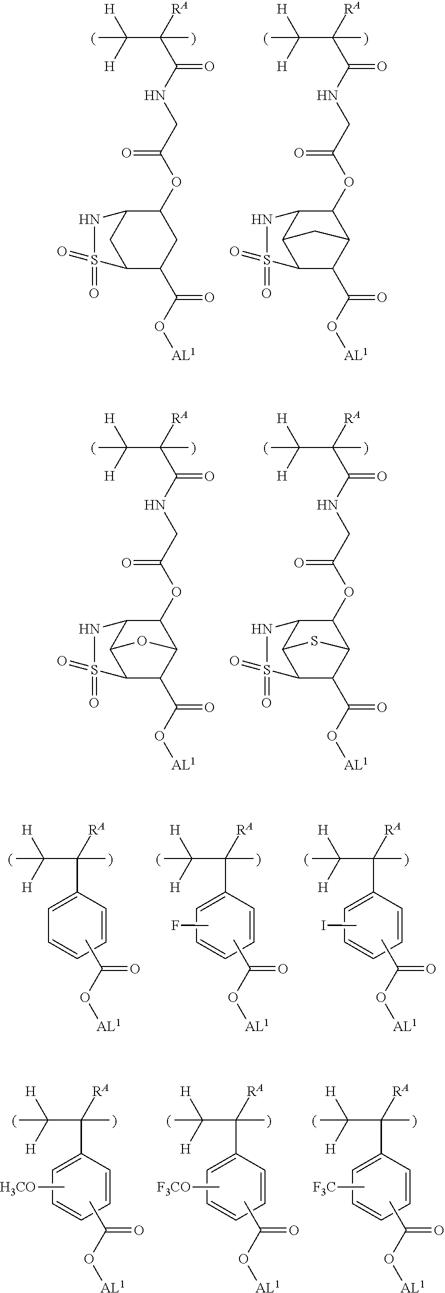

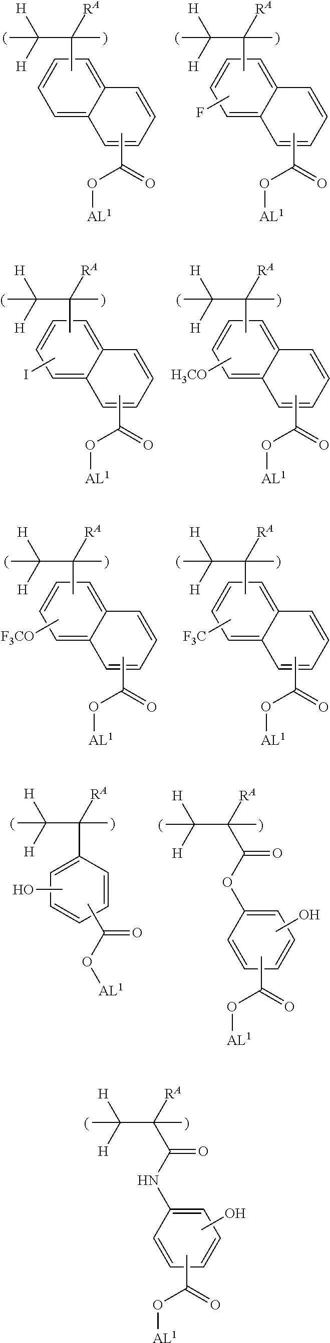

Specific examples of the repeating unit-b2 include the following, but are not limited thereto. Note that, in the following formulae, R^{A} and AL² are as defined above.

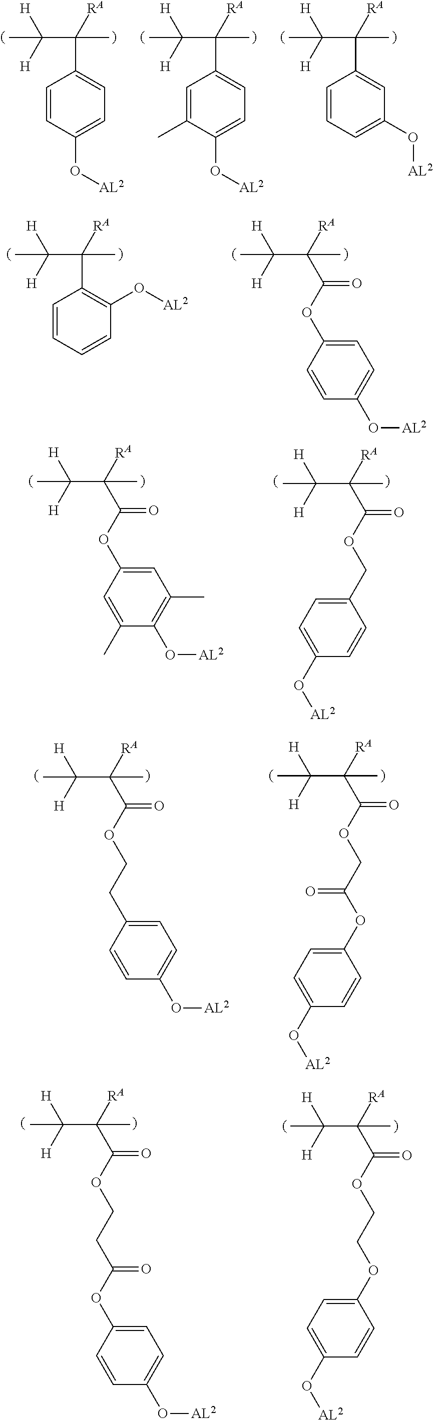

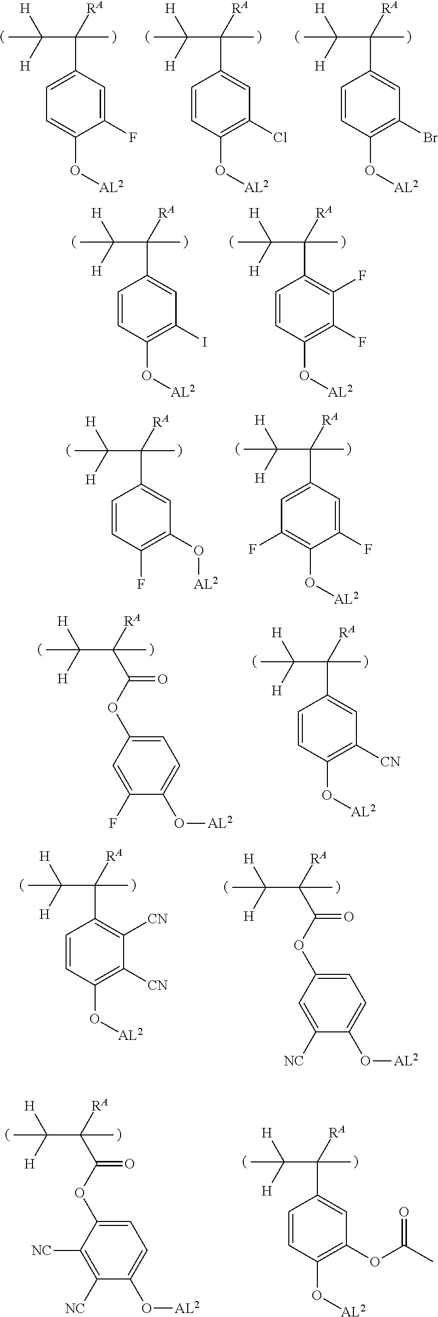

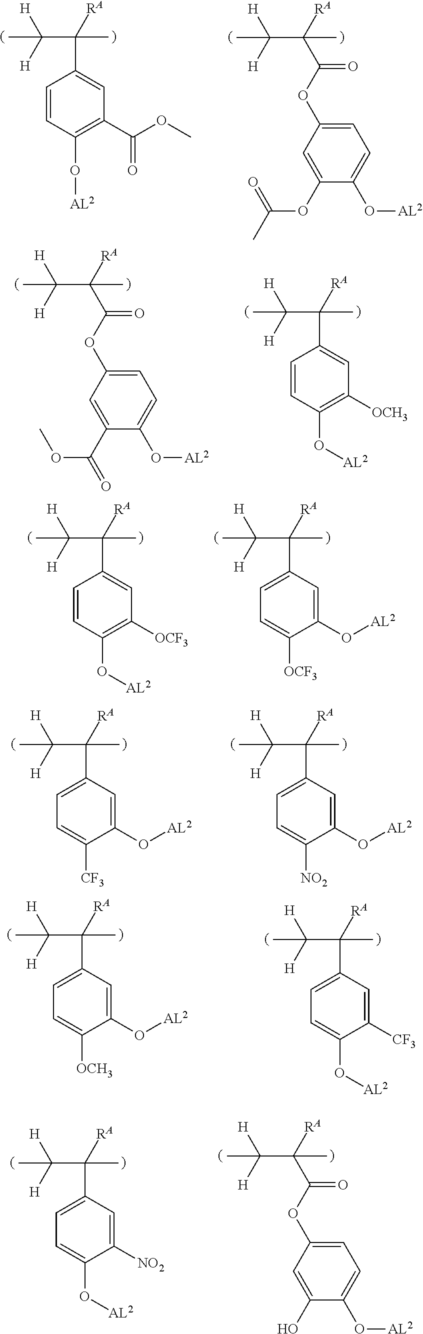

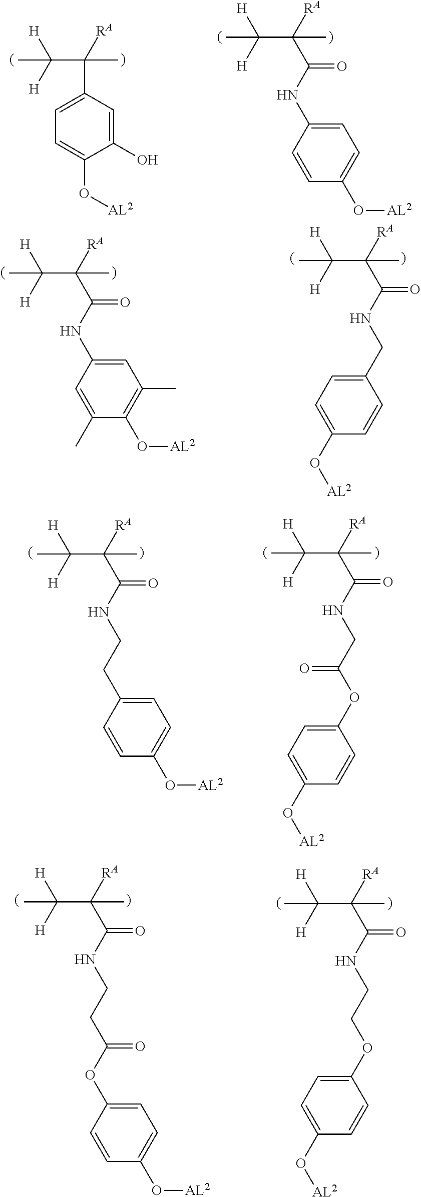

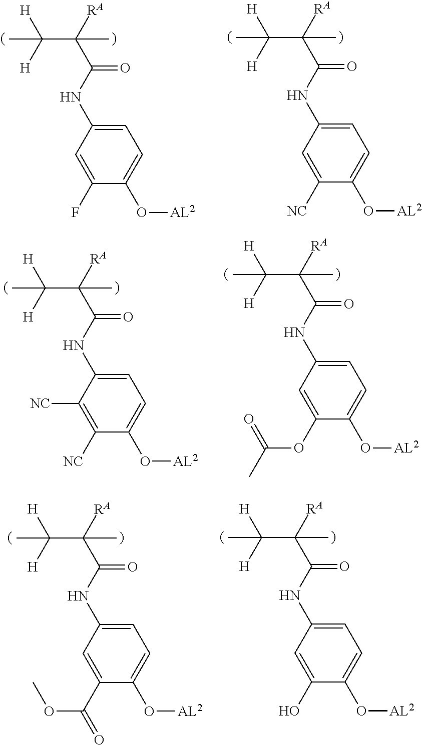

The polymer may further include a repeating unit represented by the following formula (b3) (hereinafter, also referred to as repeating unit-b3).

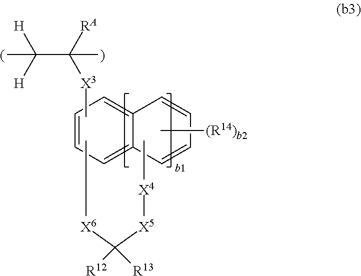

In the formula (b3), “b1” represents 0 or 1. When “b1” is 0, a benzene ring is indicated and when “b1” is 1, a naphthalene ring is indicated, and from the viewpoint of solvent solubility, a benzene ring, where “b1” is 0, is preferable. “b2” represents 0, 1, 2, or 3 when “b1” is 0 and represents 0, 1, 2, 3, 4, or 5 when “b1” is 1. From the viewpoint of procuring raw materials, “b2” is preferably 0, 1, 2, or 3, more preferably 0, 1, or 2.

In the formula (b3), R^{A} represents a hydrogen atom, a fluorine atom, a methyl group, or a trifluoromethyl group. Among these, a hydrogen atom and a methyl group are preferable, and a hydrogen atom is further preferable.

In the formula (b3), X³ represents a single bond, *—C(═O)—O—, or *—C(═O)—NH—, and “*” represents an attachment point to the carbon atom of the main chain. Among these, a single bond and *—C(═O)—O— are preferable, and a single bond is further preferable.

In the formula (b3), R¹² and R¹³ each independently represent a hydrogen atom or a hydrocarbyl group having 1 to 20 carbon atoms and optionally containing a heteroatom. The hydrocarbyl group may be saturated or unsaturated, and may be linear, branched, or cyclic. Specific examples thereof include: alkyl groups having 1 to 20 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; cyclic saturated hydrocarbyl groups having 3 to 20 carbon atoms, such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; alkenyl groups having 2 to 20 carbon atoms, such as a vinyl group, an allyl group, a propenyl group, a butenyl group, and a hexenyl group; cyclic unsaturated hydrocarbyl groups having 3 to 20 carbon atoms, such as a cyclohexenyl group; aryl groups having 6 to 20 carbon atoms, such as a phenyl group and a naphthyl group; aralkyl groups having 7 to 20 carbon atoms, such as a benzyl group, a 1-phenylethyl group, and a 2-phenylethyl group; groups which are combinations of these groups; etc. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Furthermore, R¹² and R¹³ are optionally bonded to each other to form a ring together with the carbon atom bonded thereto. Specific examples of the ring formed in this case include a cyclopropane ring, a cyclobutane ring, a cyclopentane ring, a cyclohexane ring, a norbornane ring, and an adamantane ring. Furthermore, part or all of the hydrogen atoms in the ring may be substituted with a group containing a heteroatom, such as an oxygen atom, a sulfur atom, a nitrogen atom, or a halogen atom, and part of the —CH₂— in the ring may be substituted with a group containing a heteroatom such as an oxygen atom, a sulfur atom, or a nitrogen atom. The resulting ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

In the formula (b3), R¹⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A})(R^{14B}), and R^{14A} and R^{14B} each independently represent a hydrogen atom or a hydrocarbyl group having 1 to 6 carbon atoms. The halogen atom is preferably a fluorine atom, a chlorine atom, a bromine atom, or an iodine atom, more preferably a fluorine atom or an iodine atom. The hydrocarbyl group and the hydrocarbyl moieties of the hydrocarbyloxy group, the hydrocarbyloxycarbonyl group, and the hydrocarbylthio group may be saturated or unsaturated, and may be linear, branched, or cyclic. Specific examples thereof include those given as examples of the hydrocarbyl groups represented by R¹² and R¹³. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc. When “b2” is 2 or more, the R¹⁴s may be identical to or different from each other.

Furthermore, when “b2” is 2 or more, the R¹⁴s are optionally bonded to each other to form a ring together with the carbon atoms of the aromatic ring bonded thereto. Specific examples of the ring formed in this case include a cyclopropane ring, a cyclobutane ring, a cyclopentane ring, a cyclohexane ring, a norbornane ring, and an adamantane ring. Furthermore, part or all of the hydrogen atoms in the ring may be substituted with a group containing a heteroatom, such as an oxygen atom, a sulfur atom, a nitrogen atom, or a halogen atom, and part of the —CH₂— in the ring may be substituted with a group containing a heteroatom such as an oxygen atom, a sulfur atom, or a nitrogen atom. The resulting ring may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

In the formula (b3), X⁴ represents a single bond, an aliphatic hydrocarbylene group having 1 to 4 carbon atoms, a carbonyl group, a sulfonyl group, or a group which is a combination of the groups. Among these, a single bond, a carbonyl group, or a sulfonyl group is preferable from the viewpoint of procuring raw materials, and a single bond or a carbonyl group is more preferable from the viewpoint of polar groups generated after a reaction.

In the formula (b3), X⁵ and X⁶ each independently represent an oxygen atom or a sulfur atom, provided that the X⁴ and the X⁶ are bonded to adjacent carbon atoms of the aromatic ring. X⁵ and X⁶ may be identical to or different from each other. However, from the viewpoint of reactivity, it is preferable that X⁵ and X⁶ are both oxygen atoms.

Specific examples of the repeating unit-b3 include the following, but are not limited thereto. Note that, in the following formulae, R^{A} is as defined above, and Me represents a methyl group. Furthermore, the attachment positions of the substituents on the aromatic rings may change places with each other.

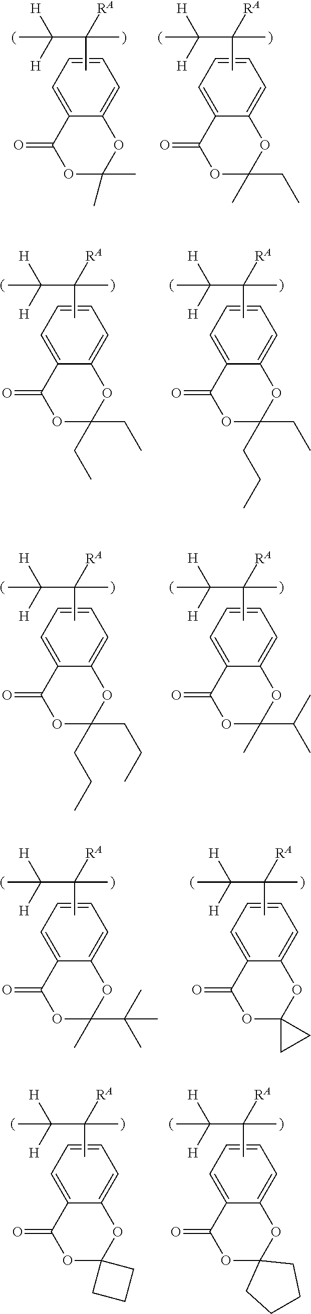

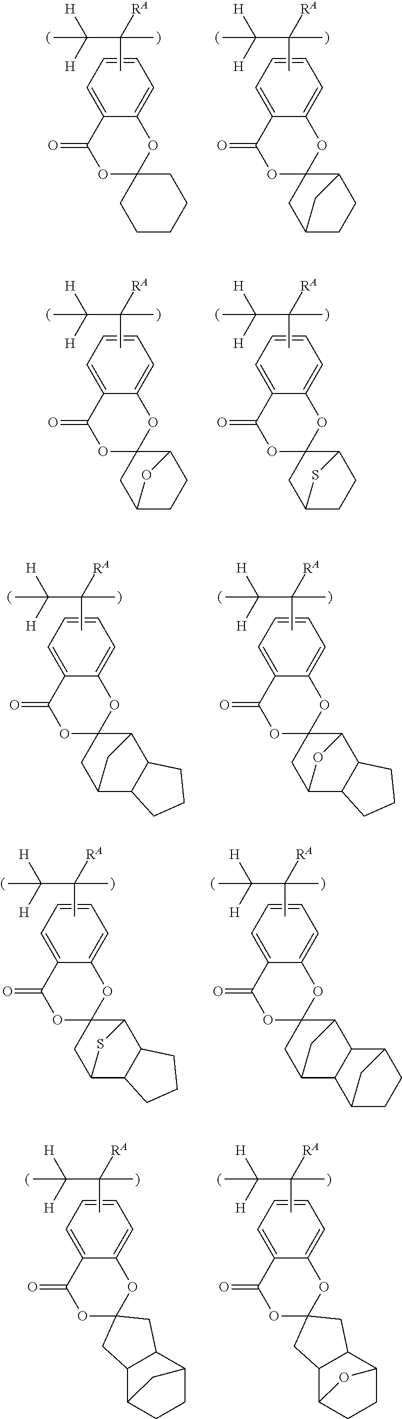

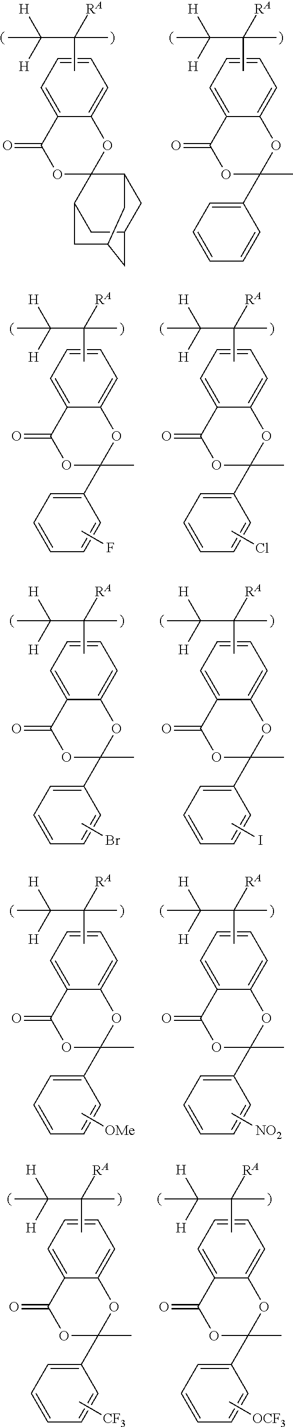

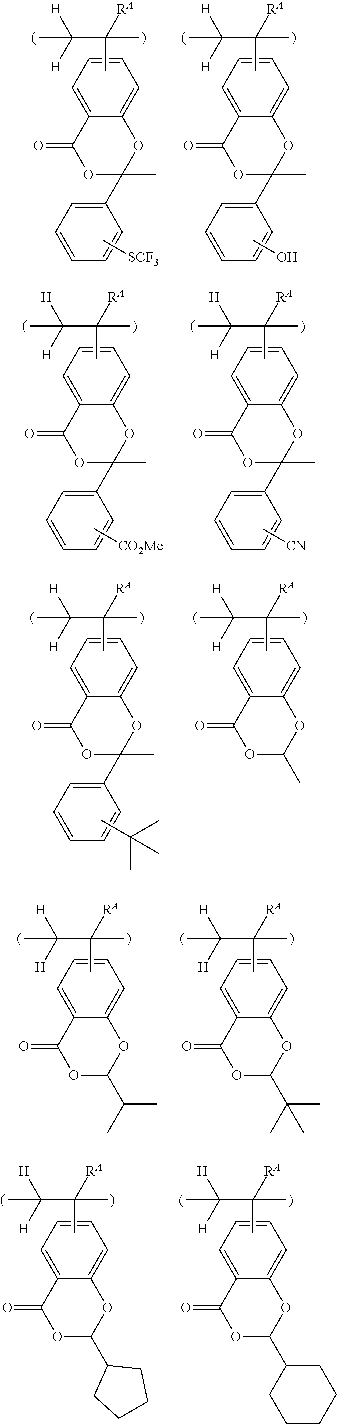

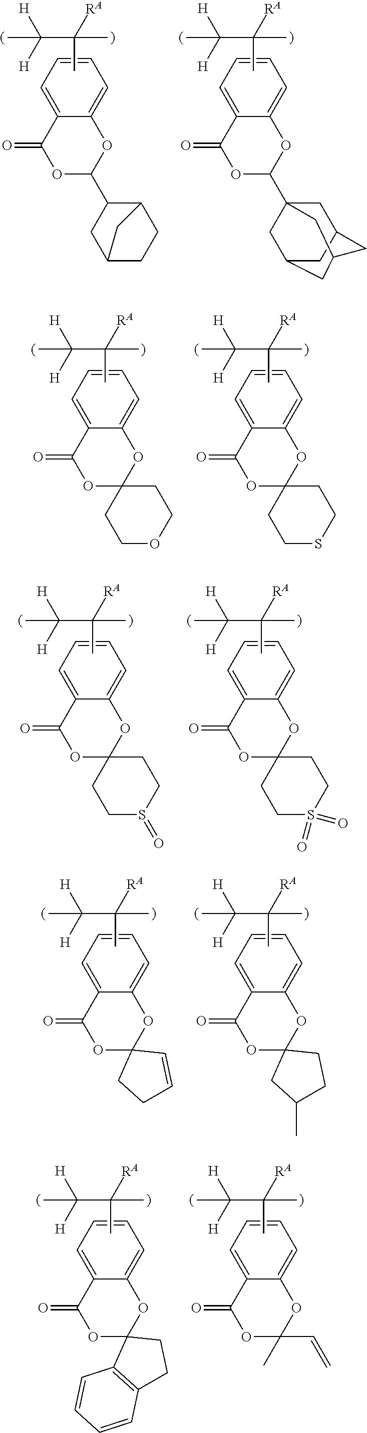

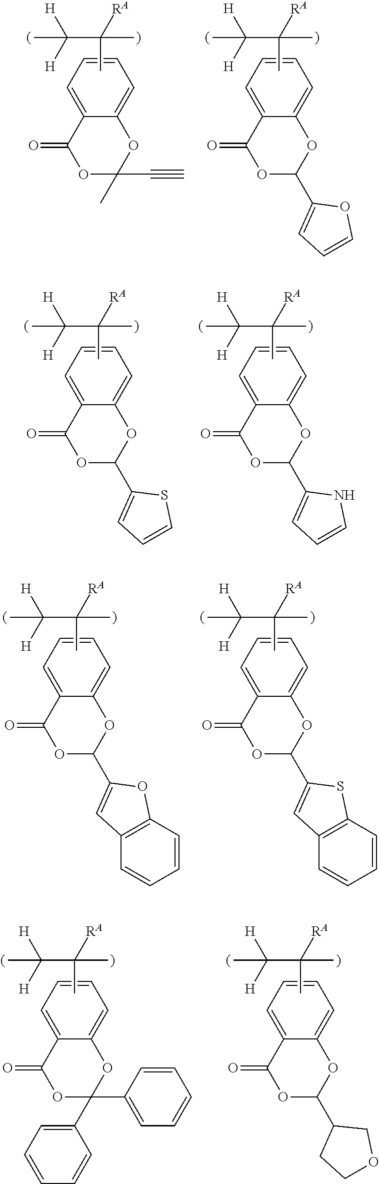

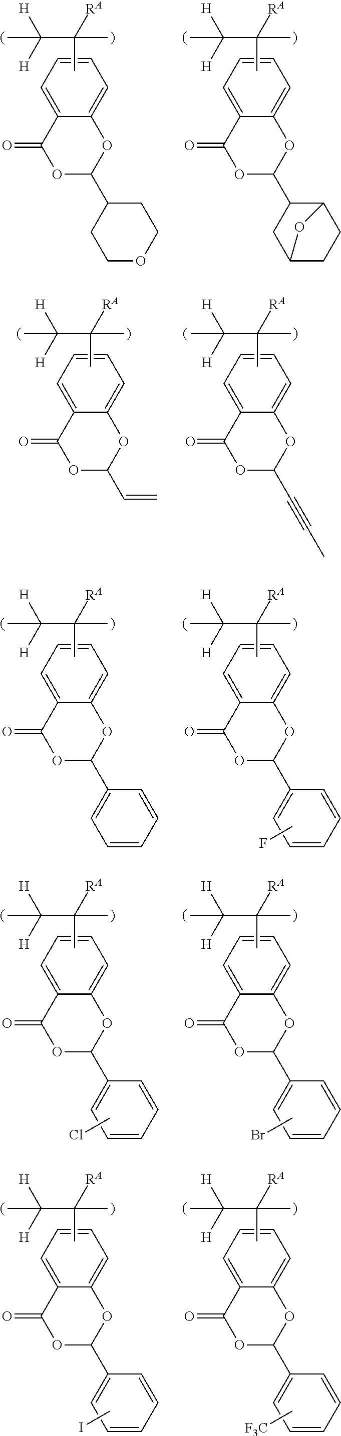

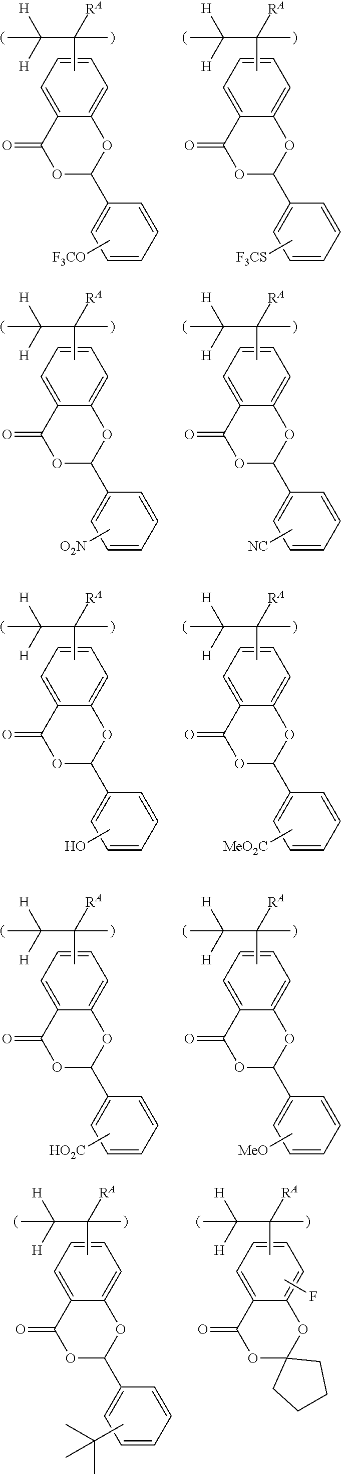

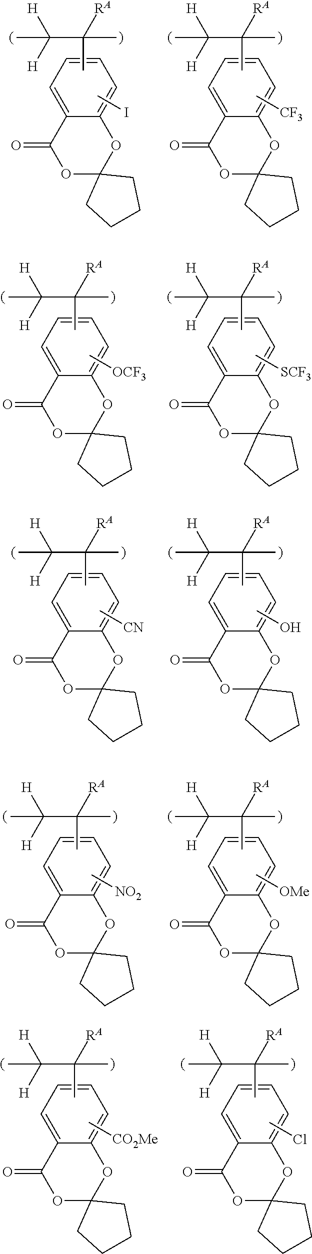

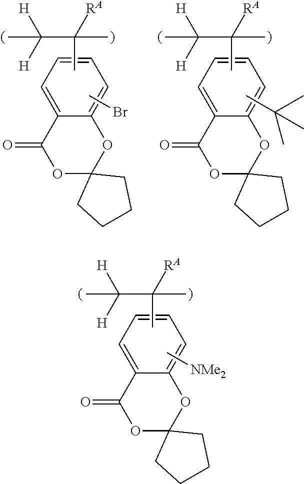

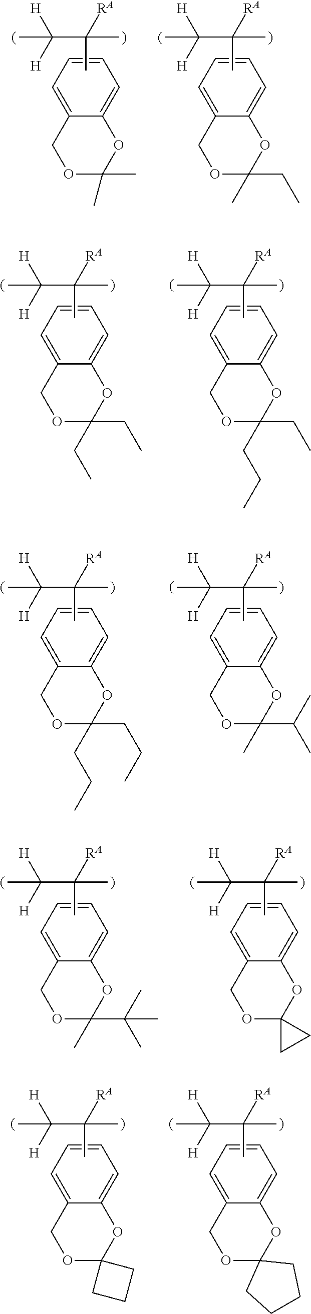

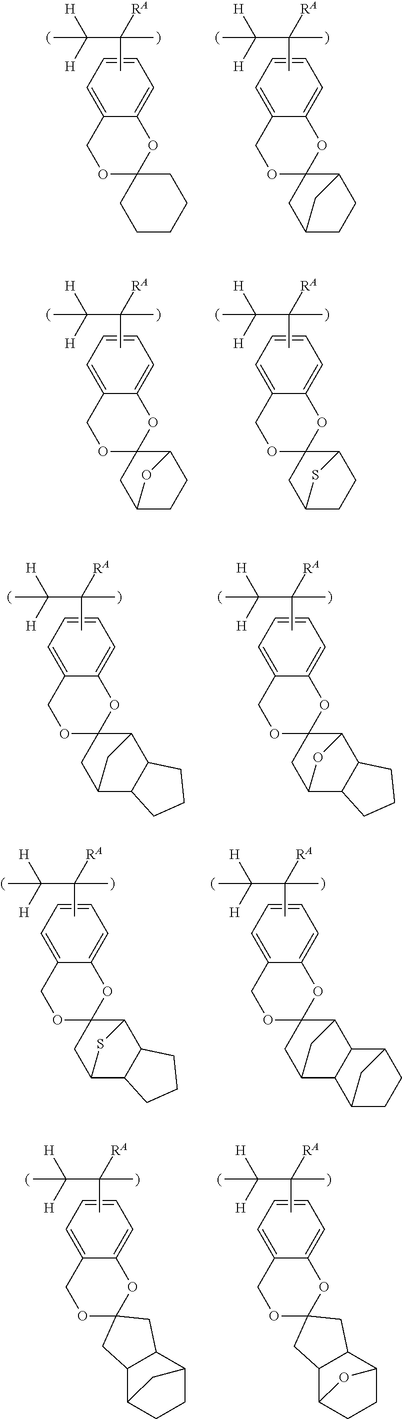

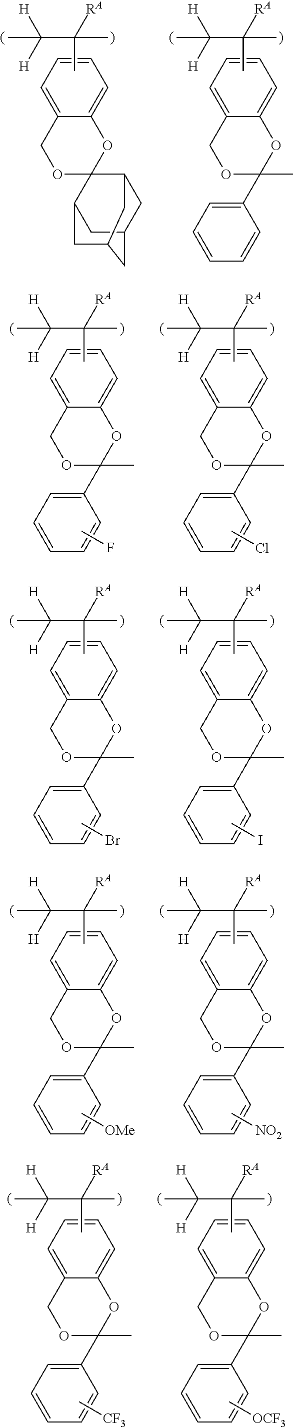

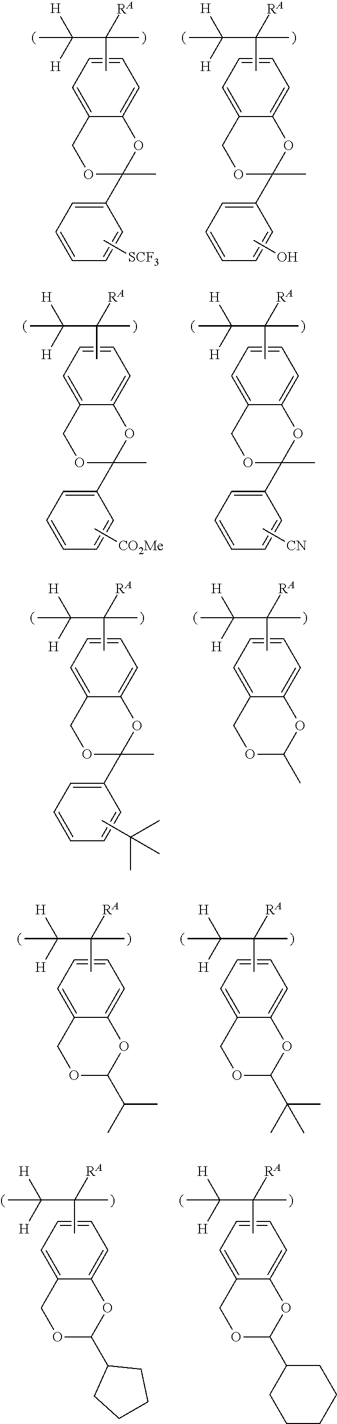

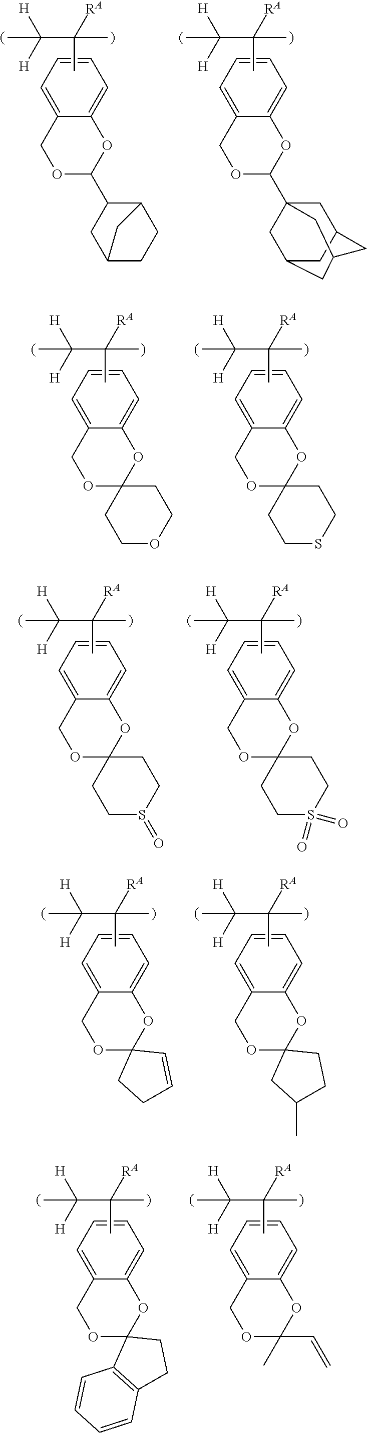

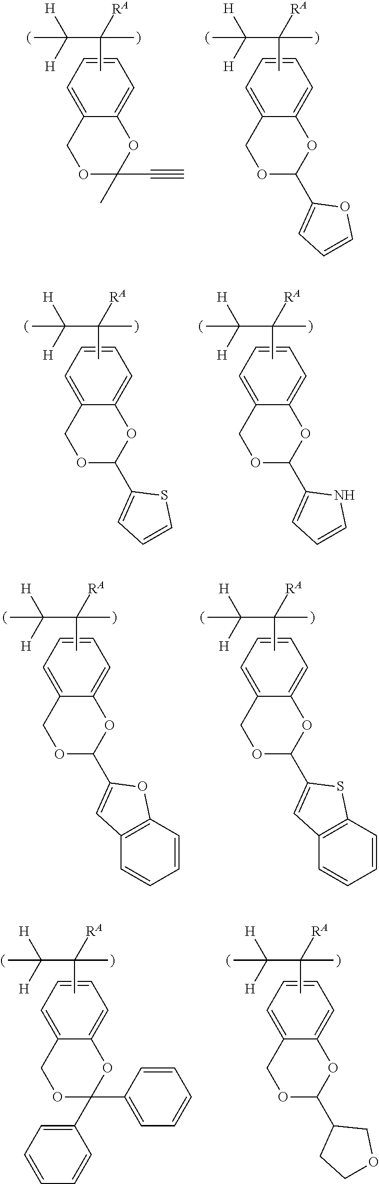

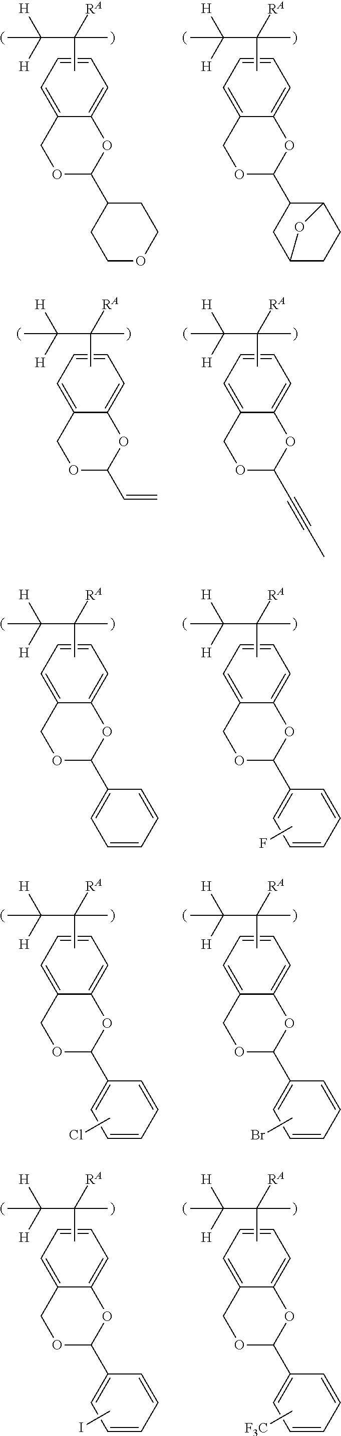

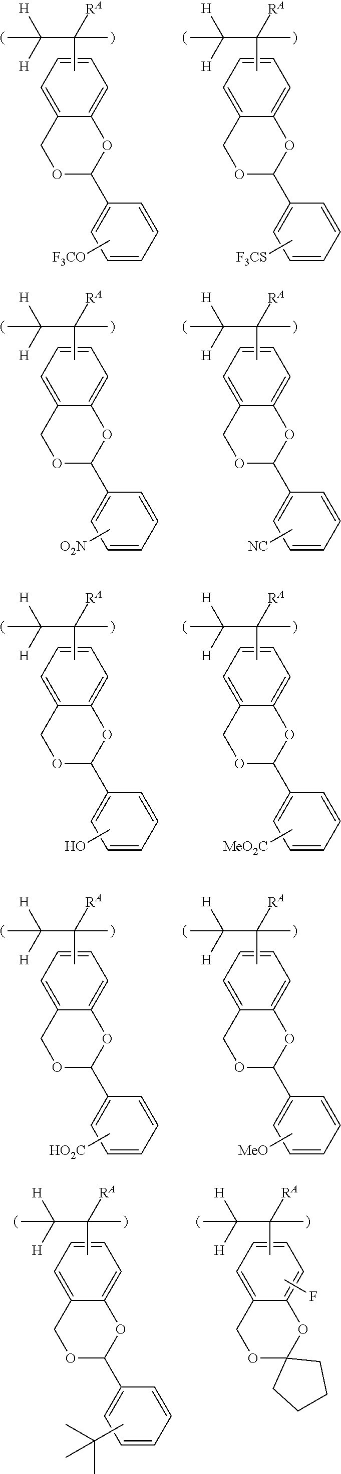

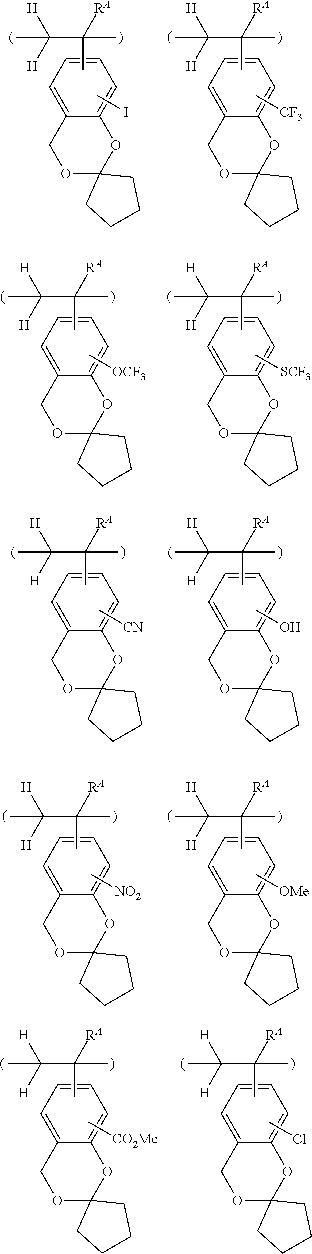

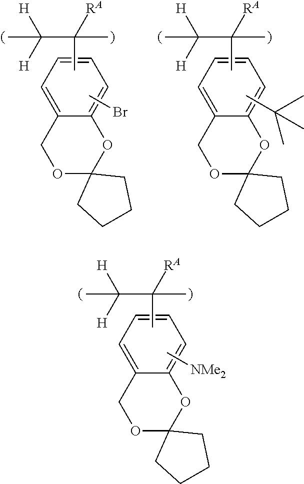

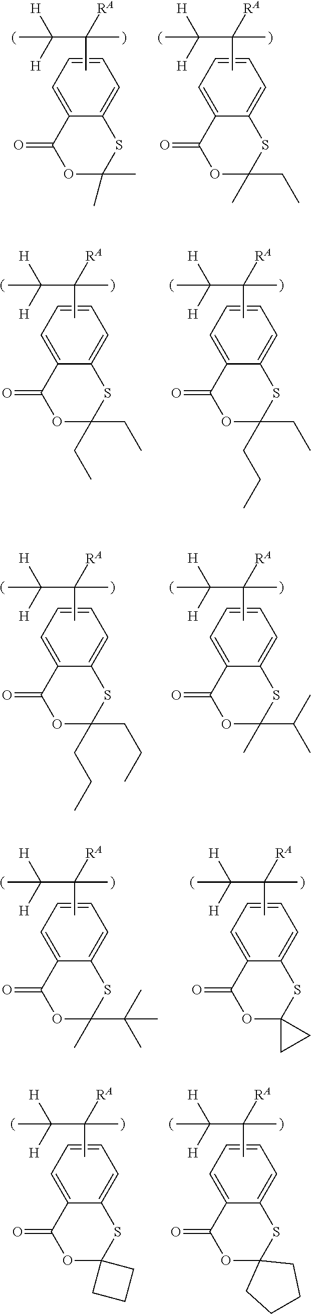

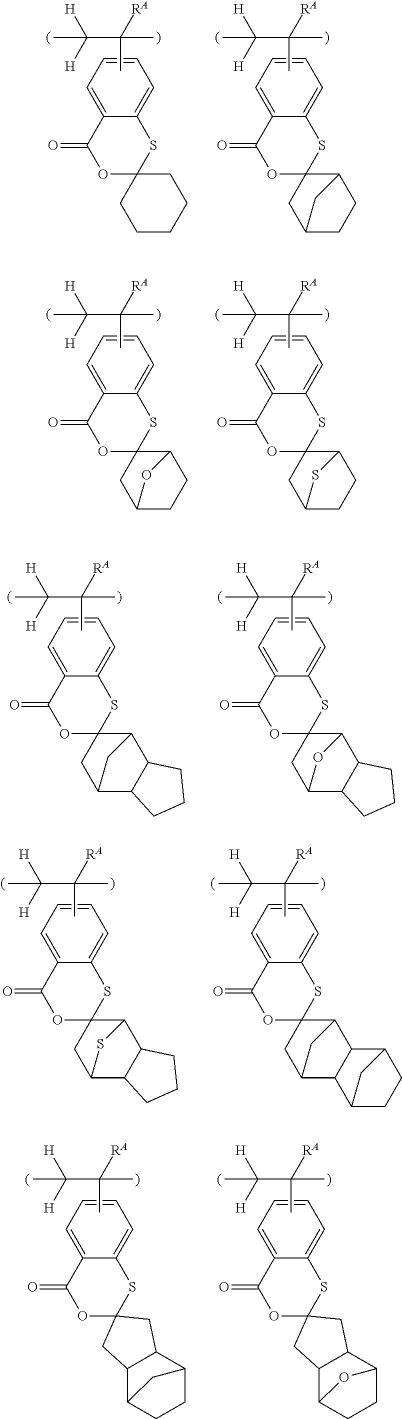

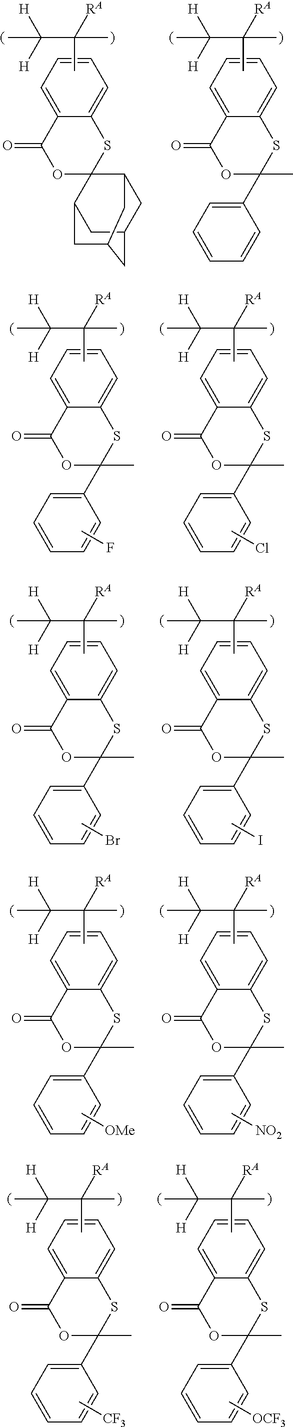

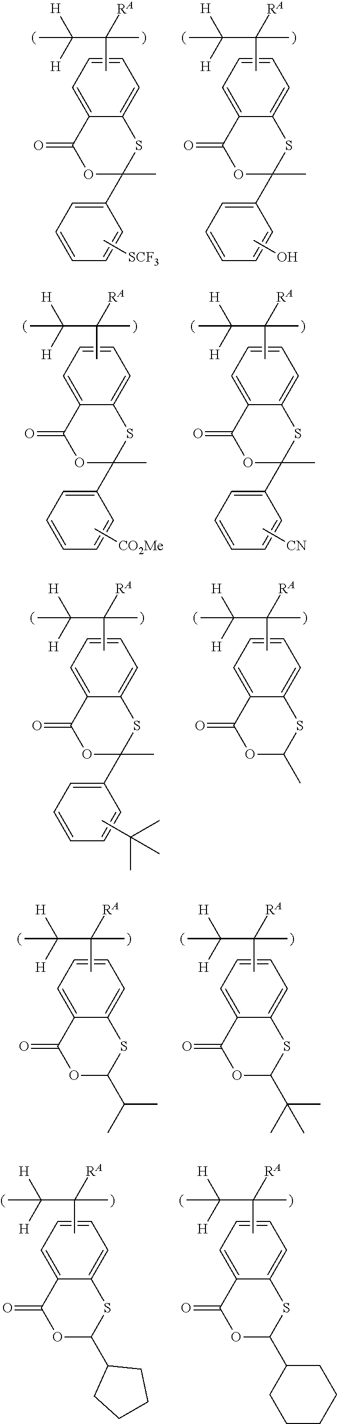

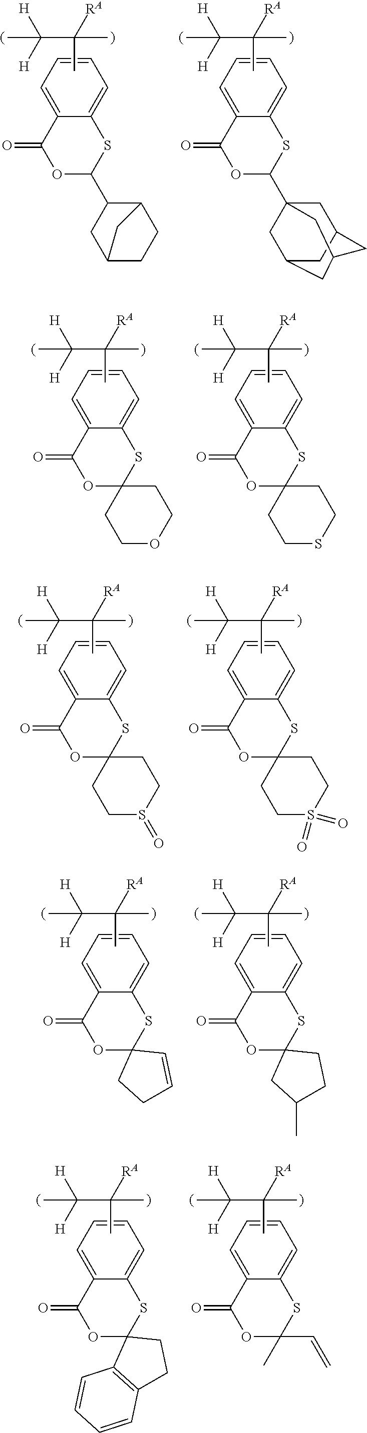

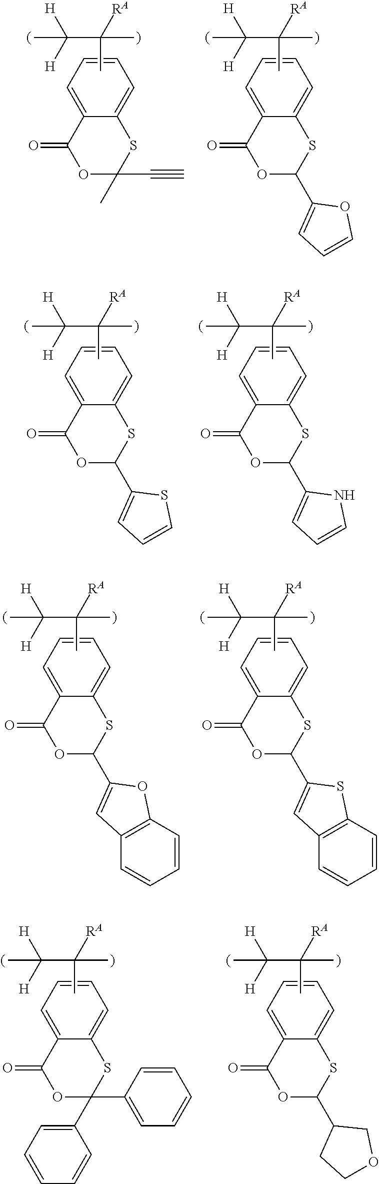

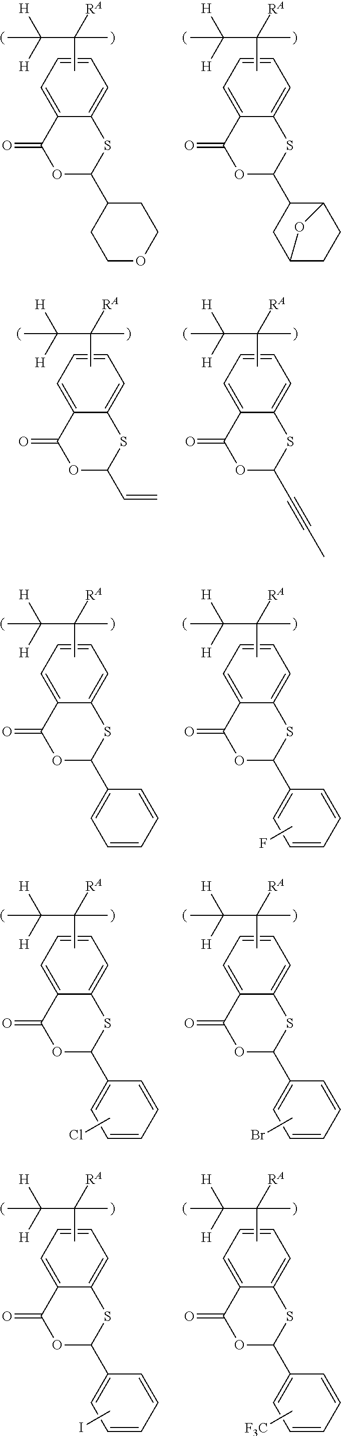

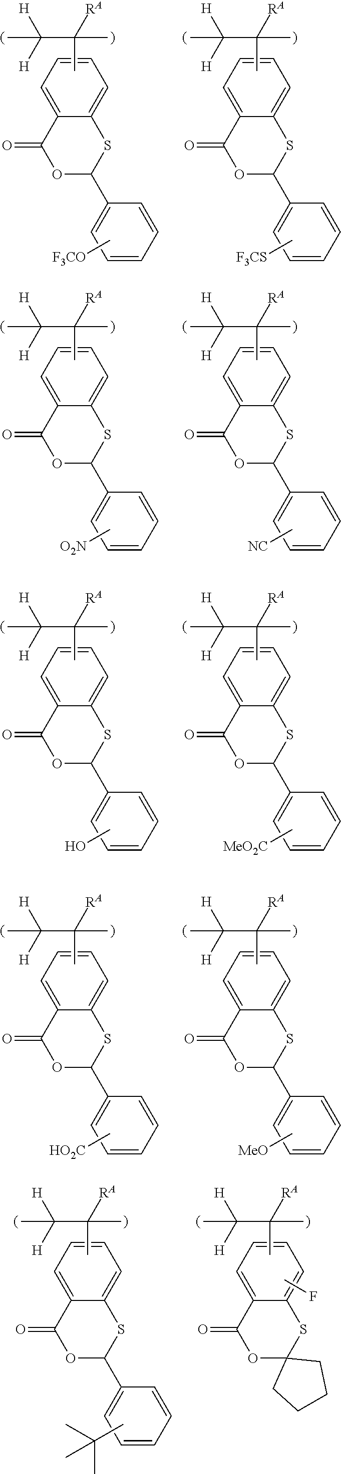

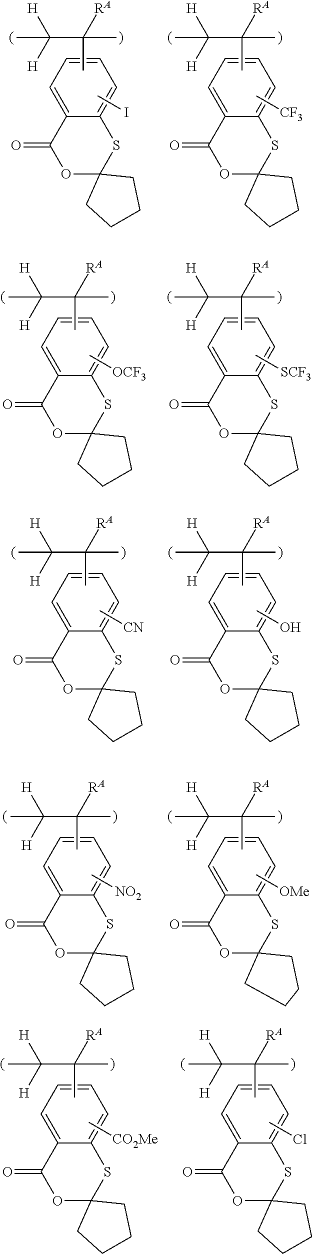

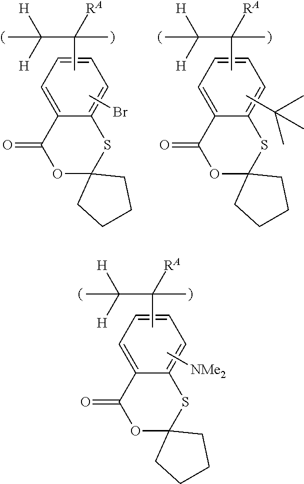

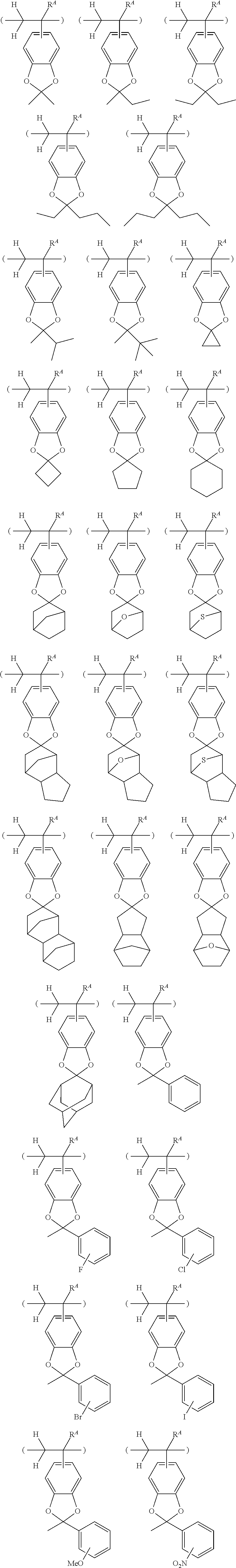

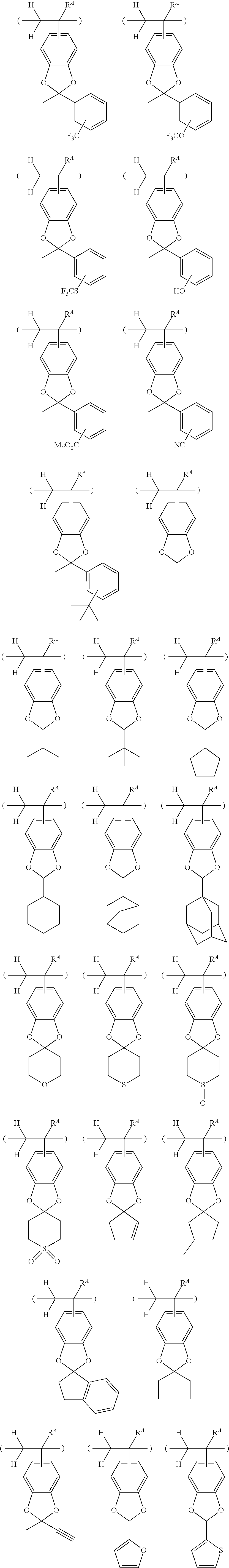

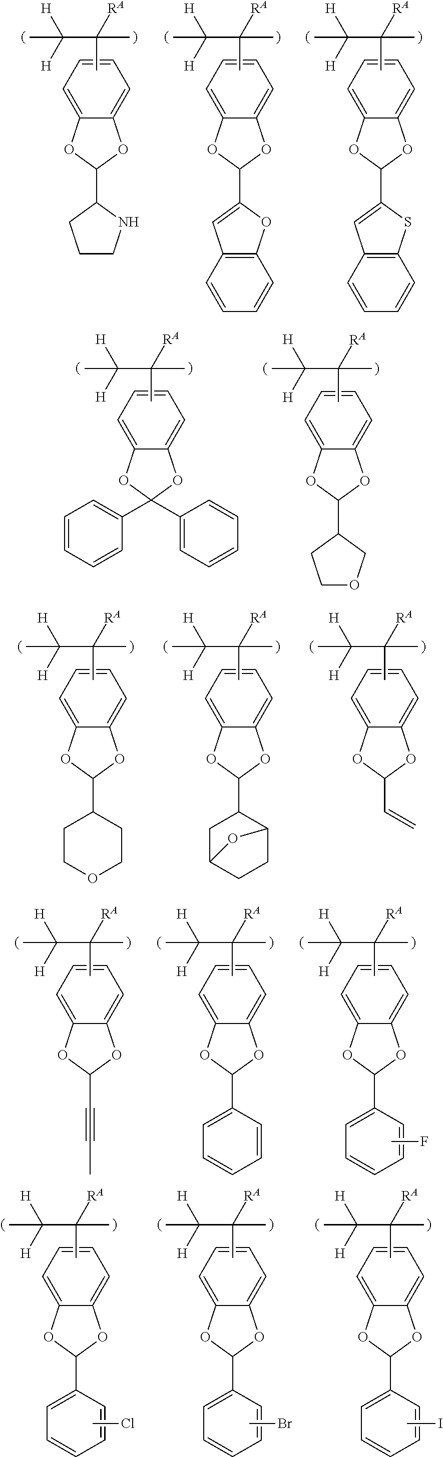

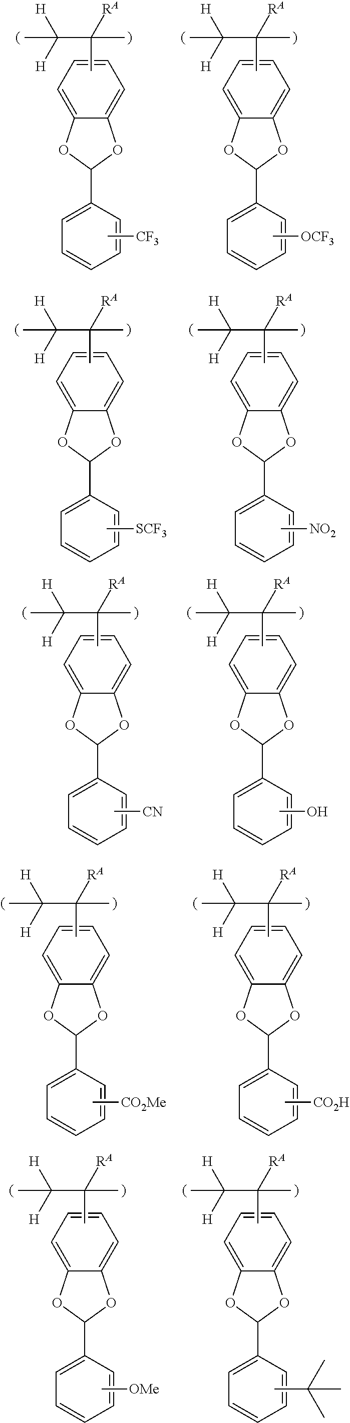

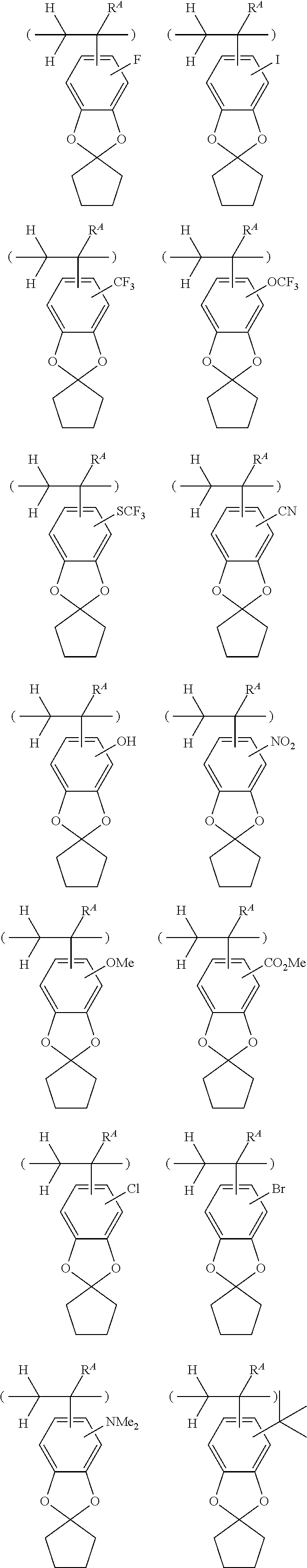

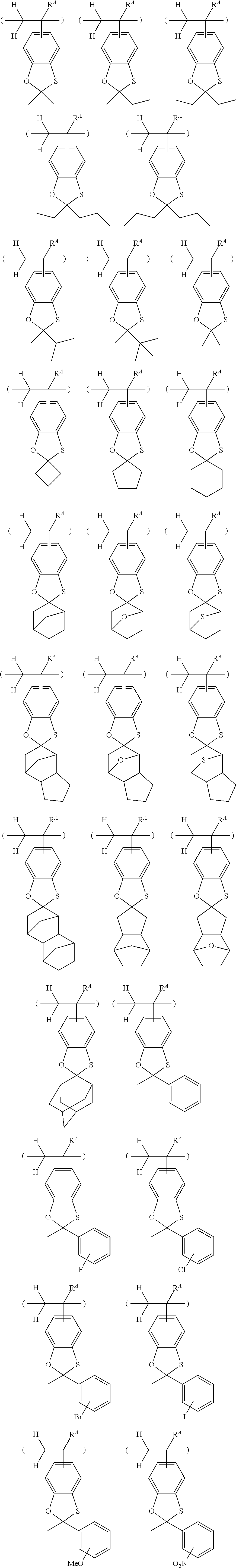

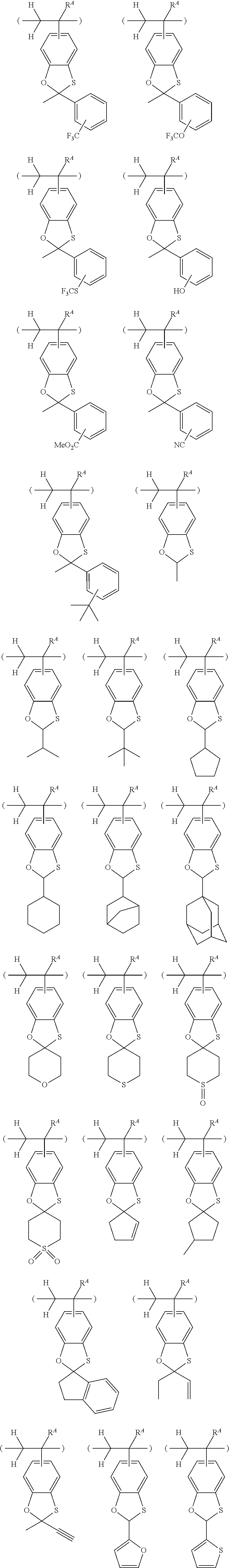

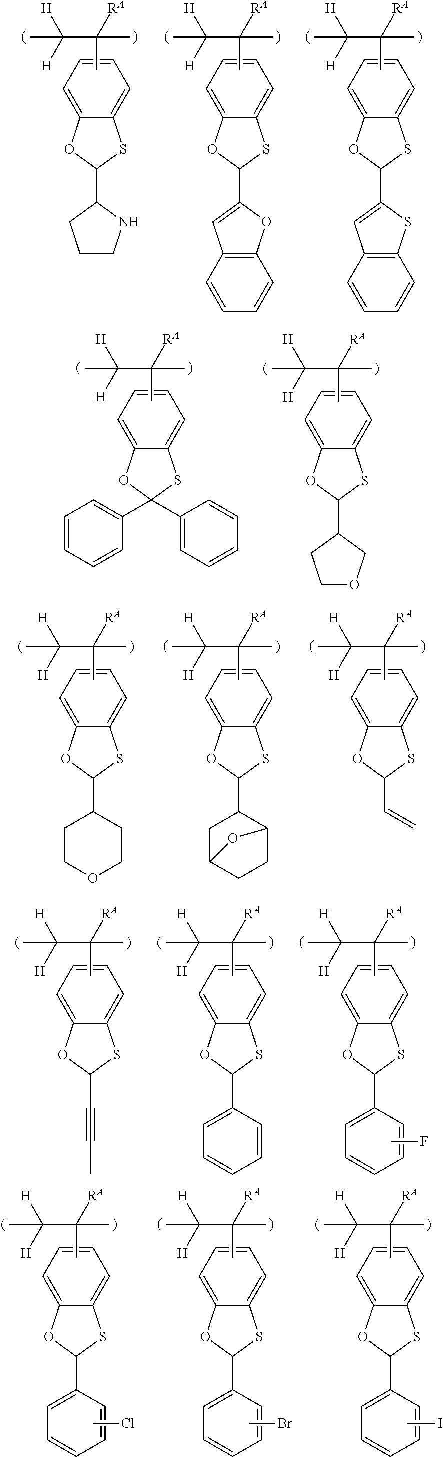

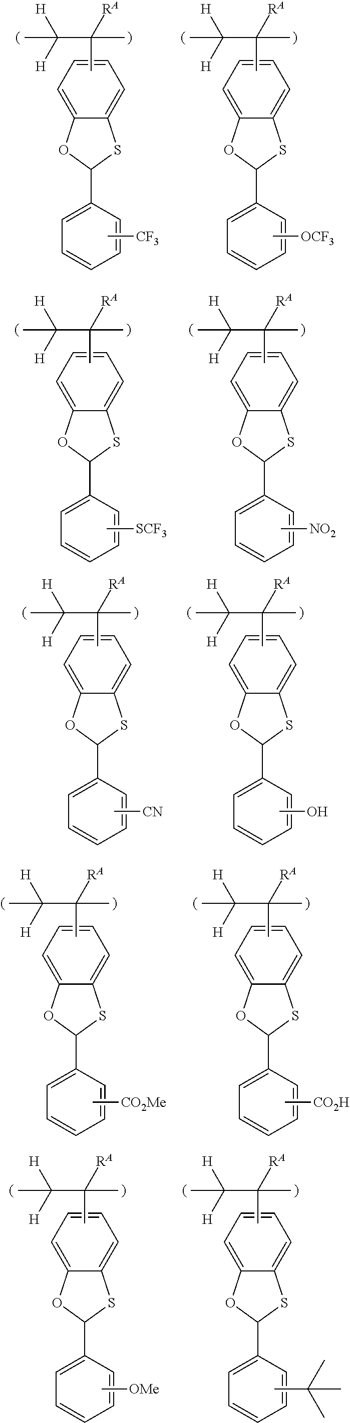

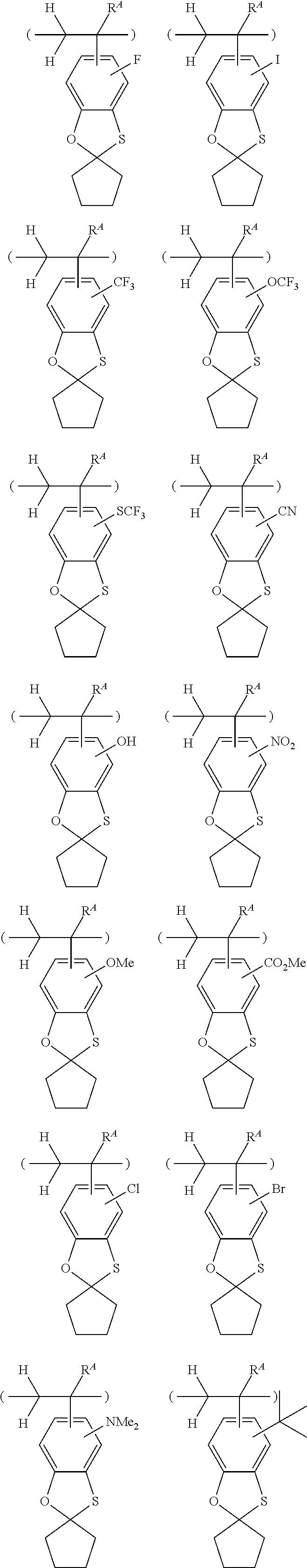

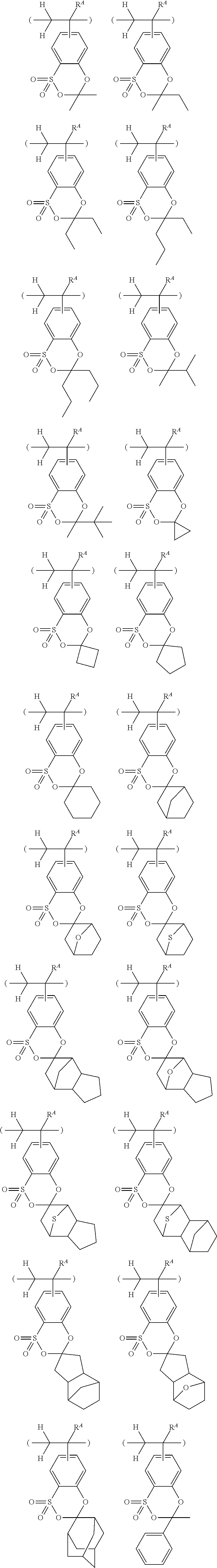

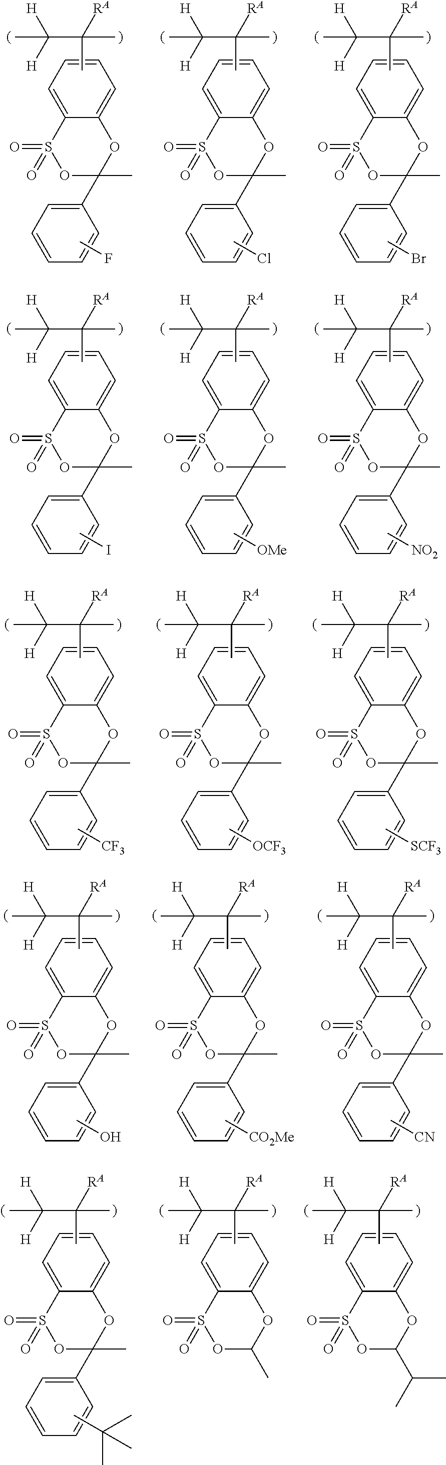

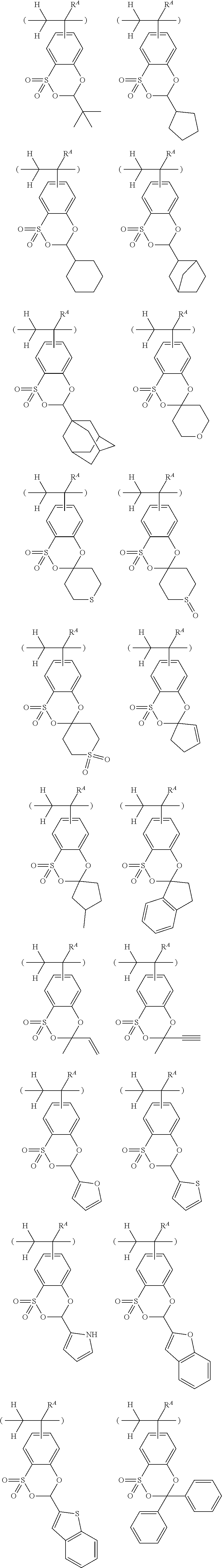

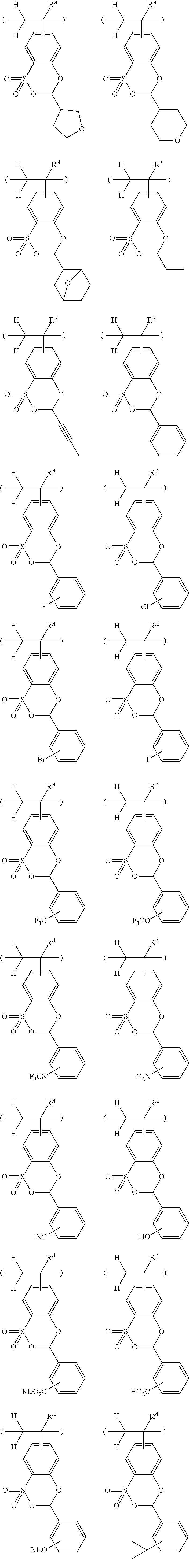

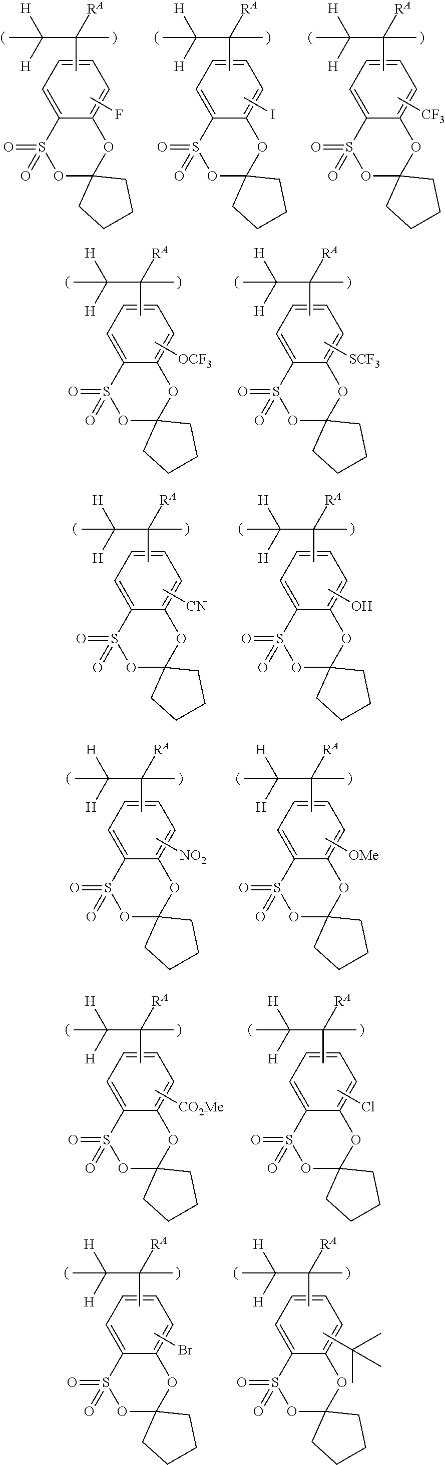

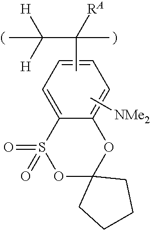

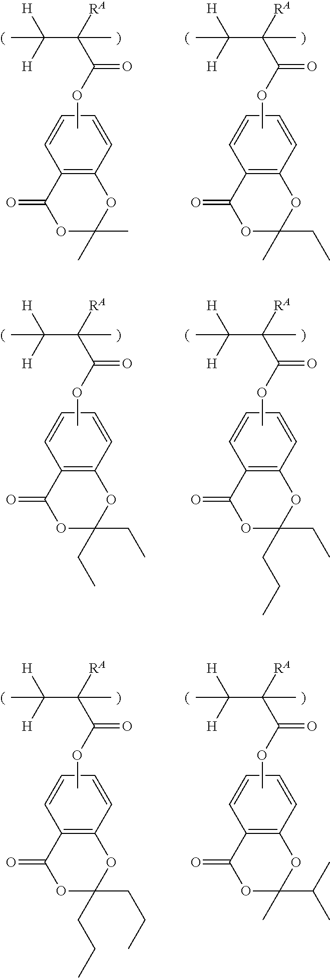

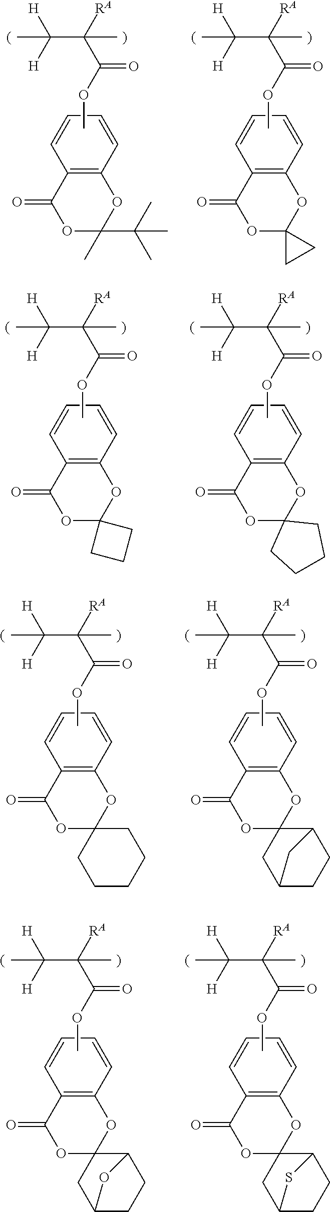

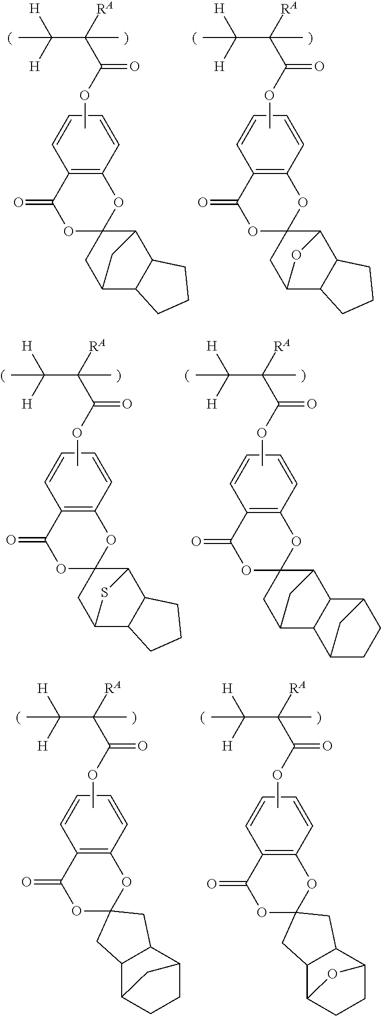

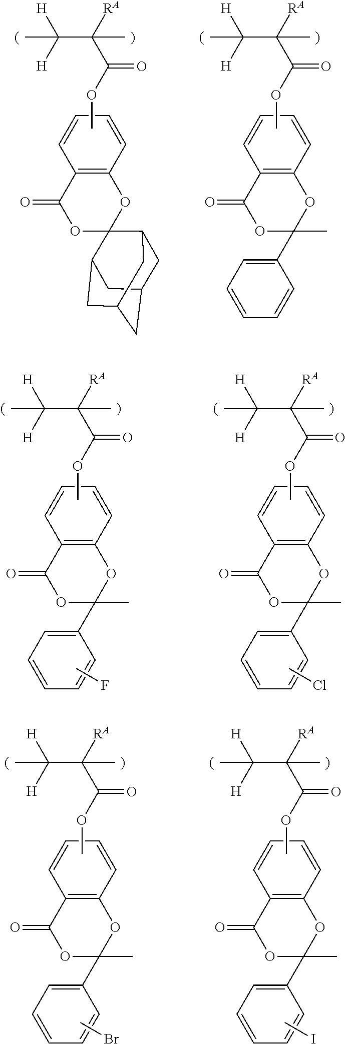

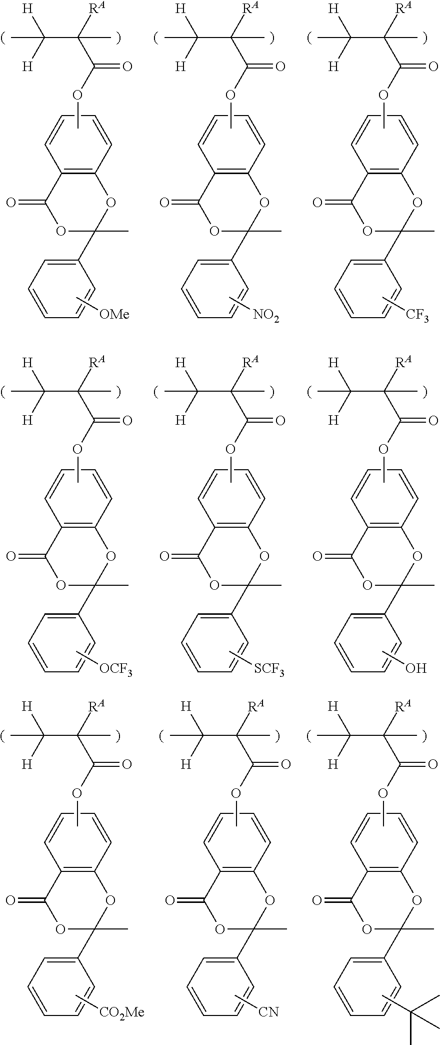

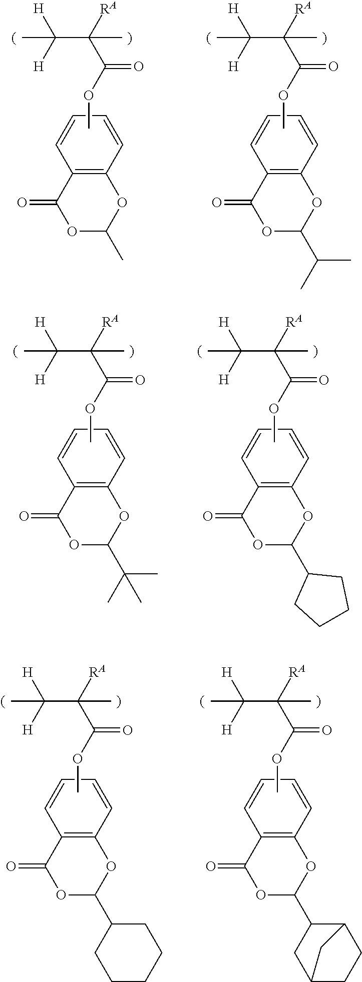

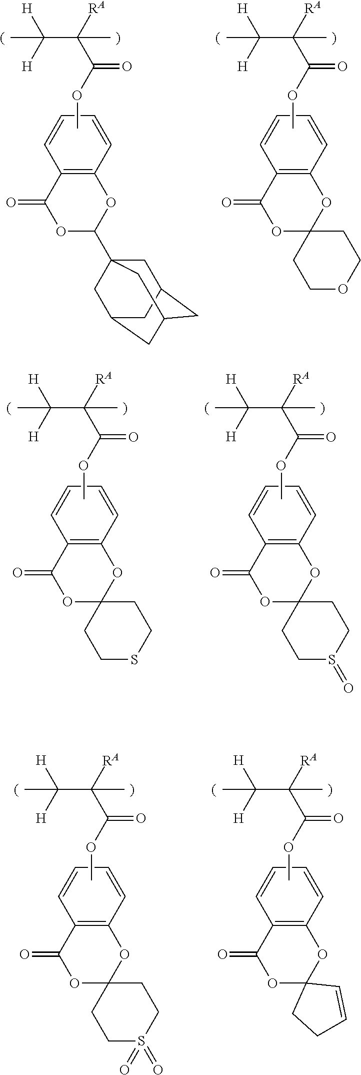

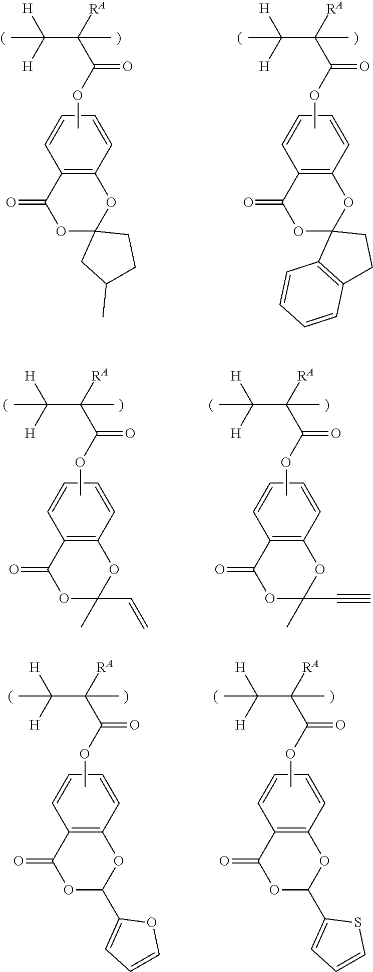

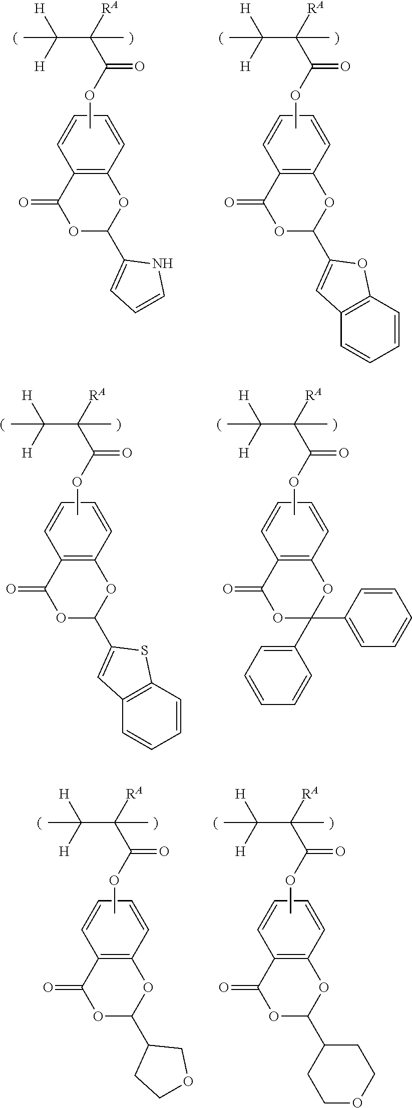

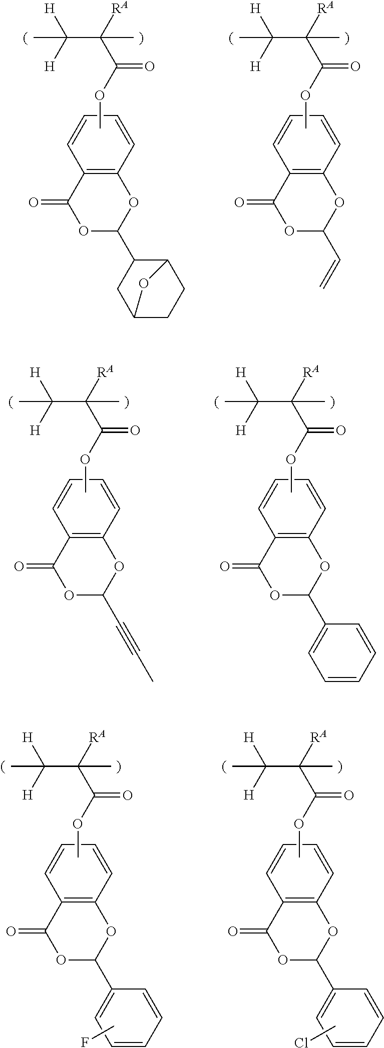

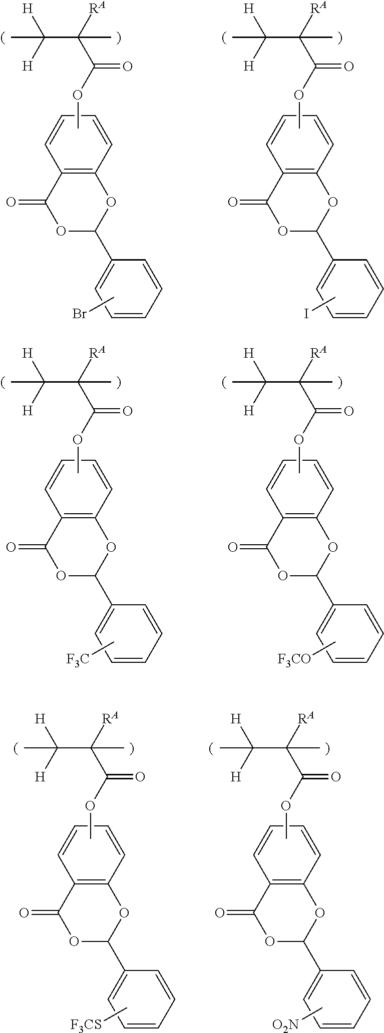

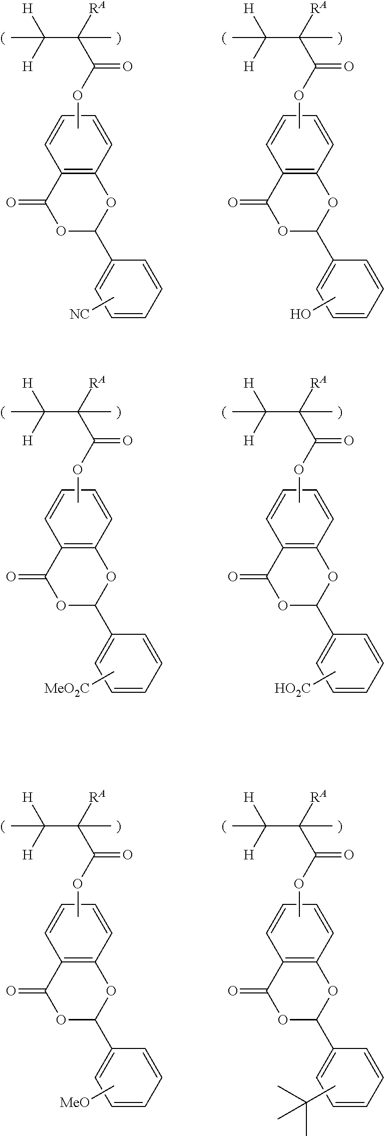

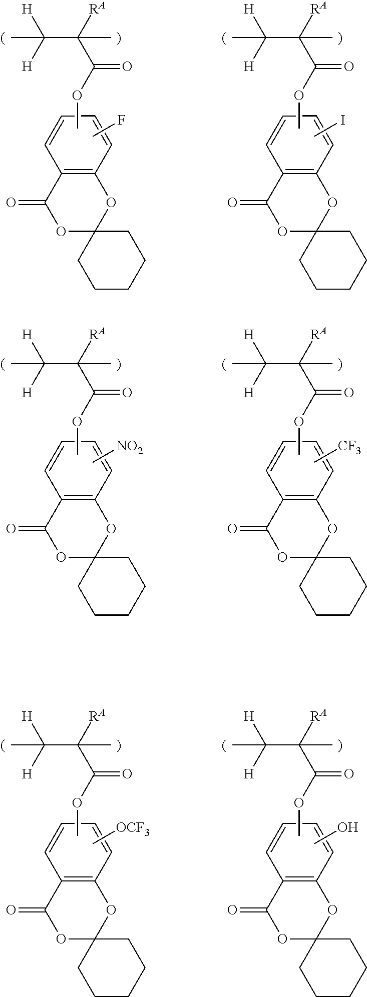

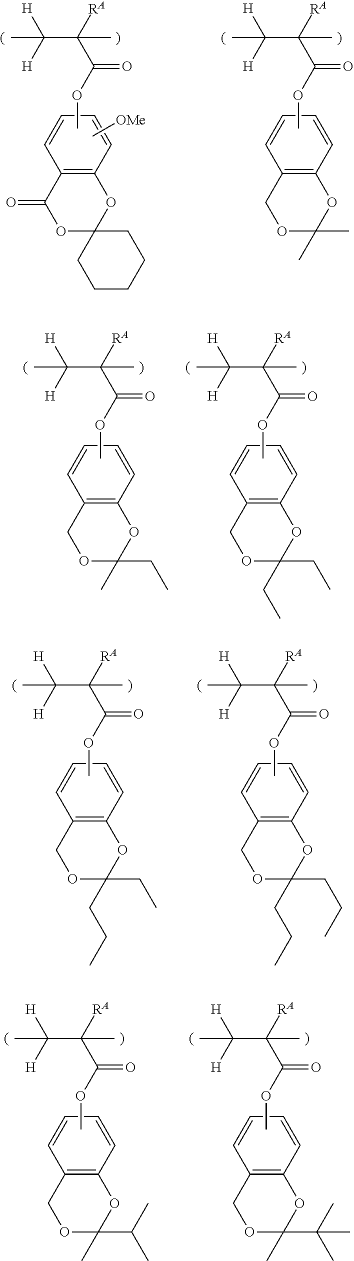

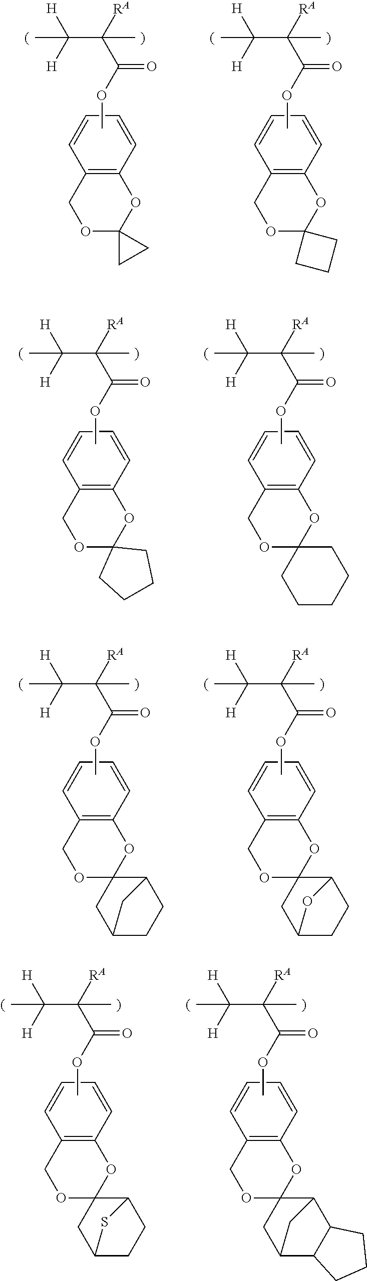

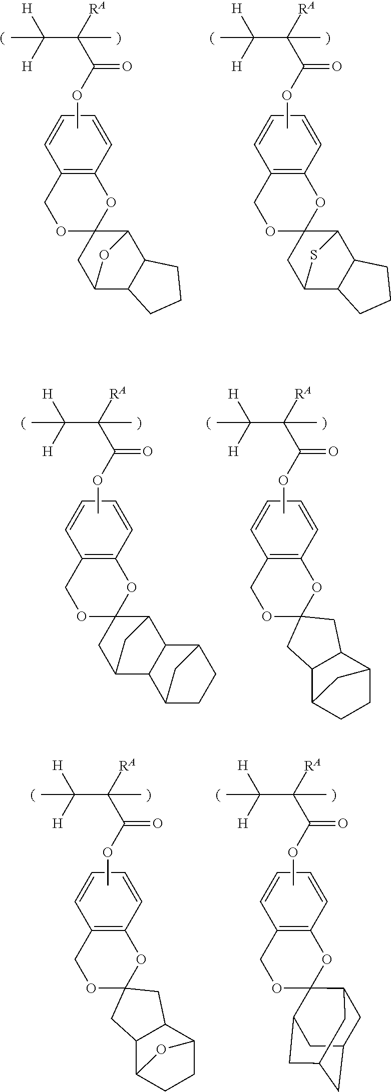

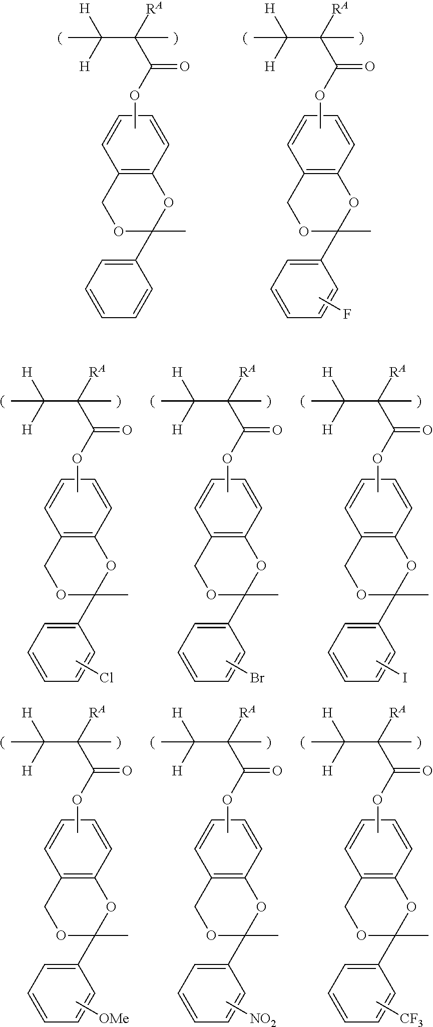

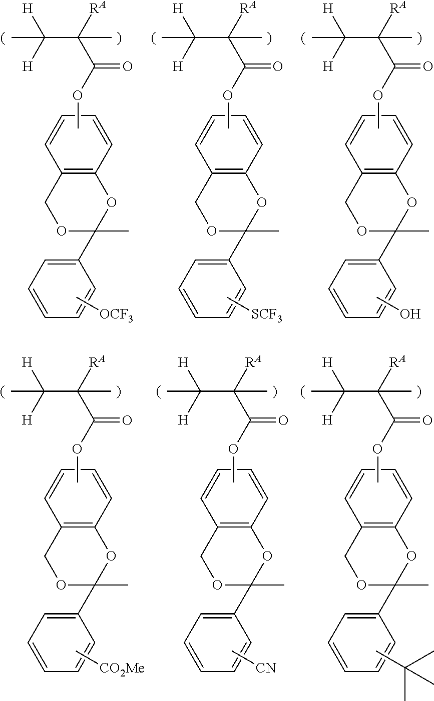

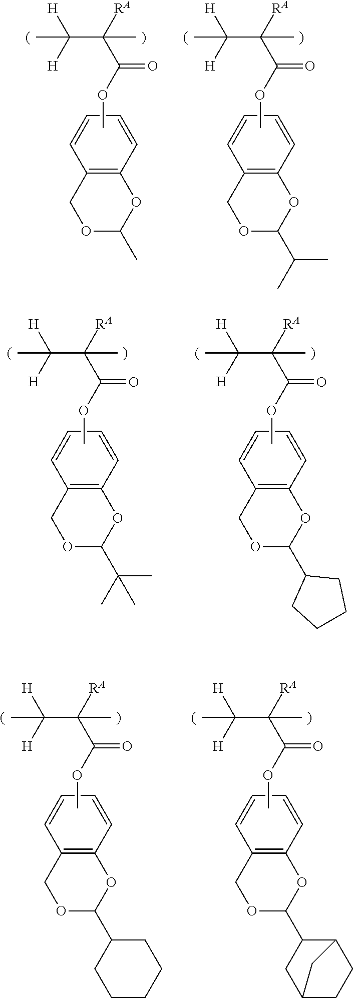

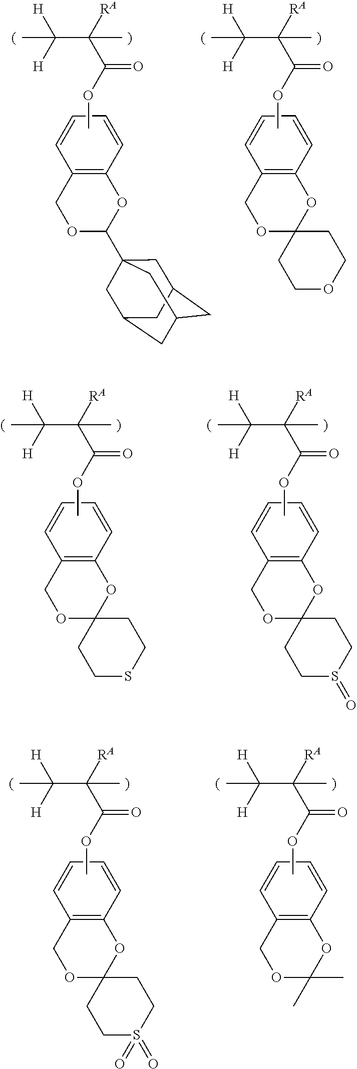

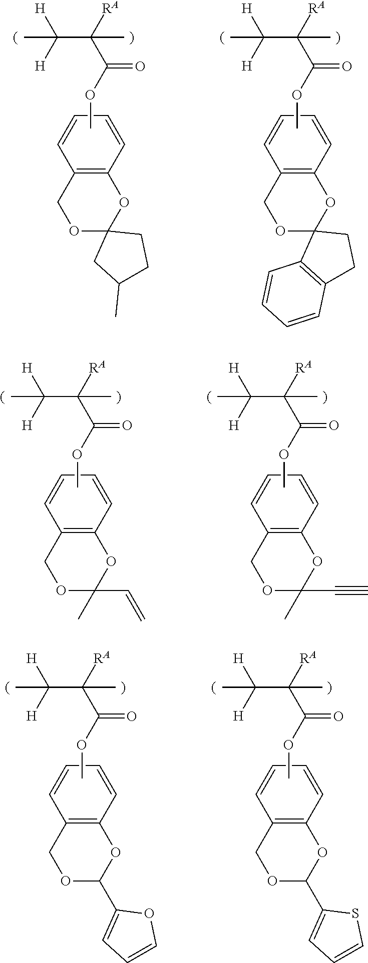

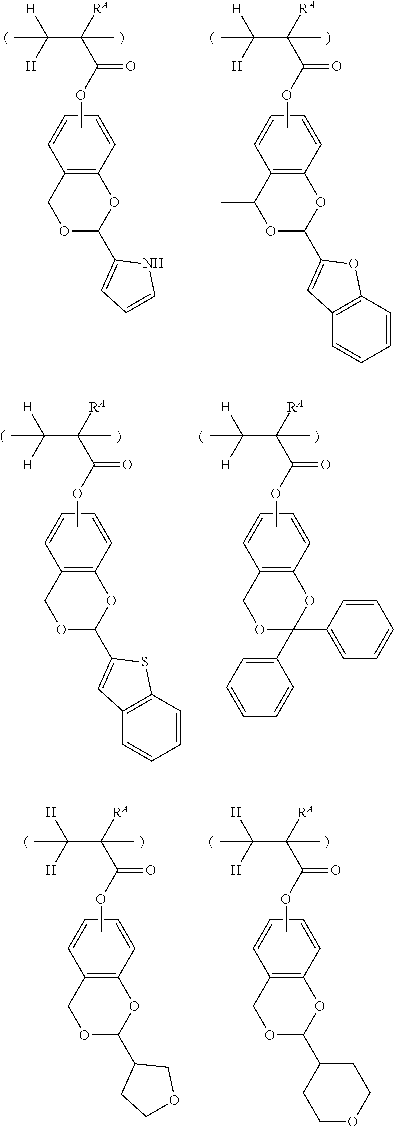

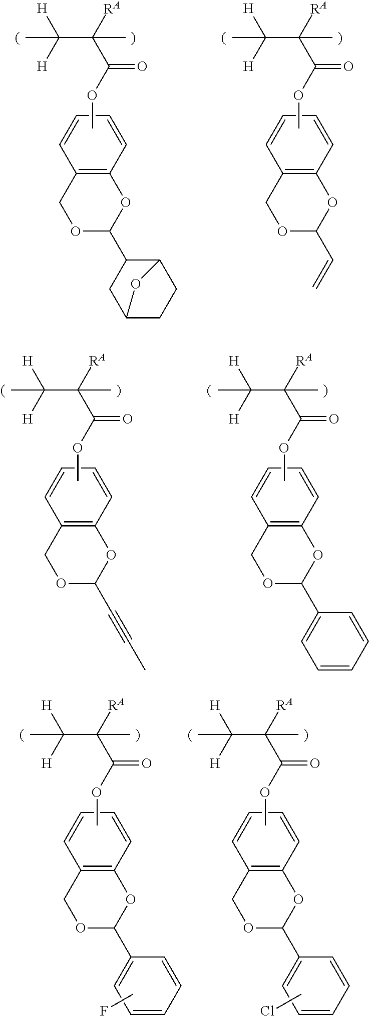

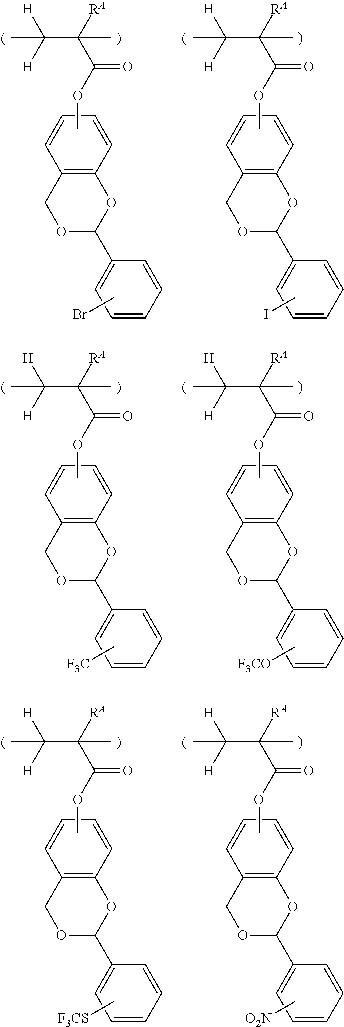

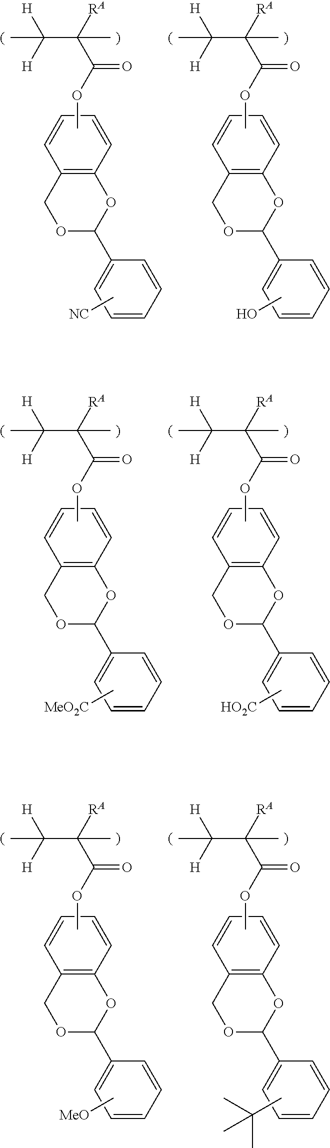

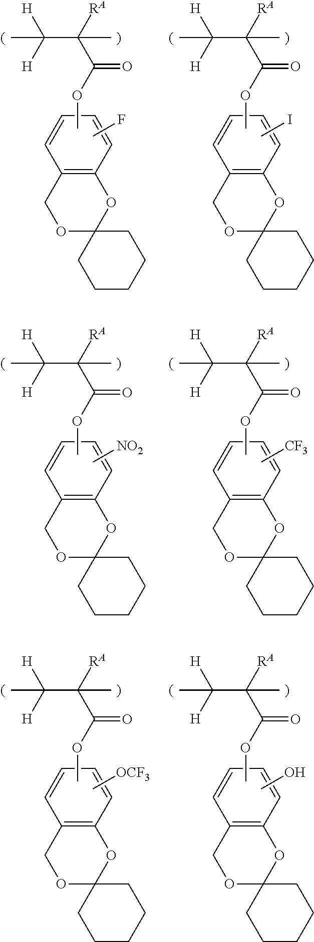

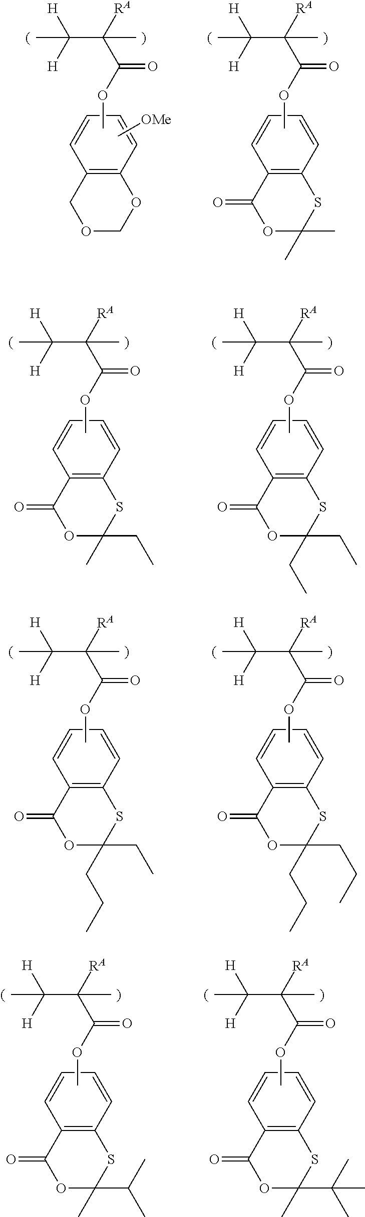

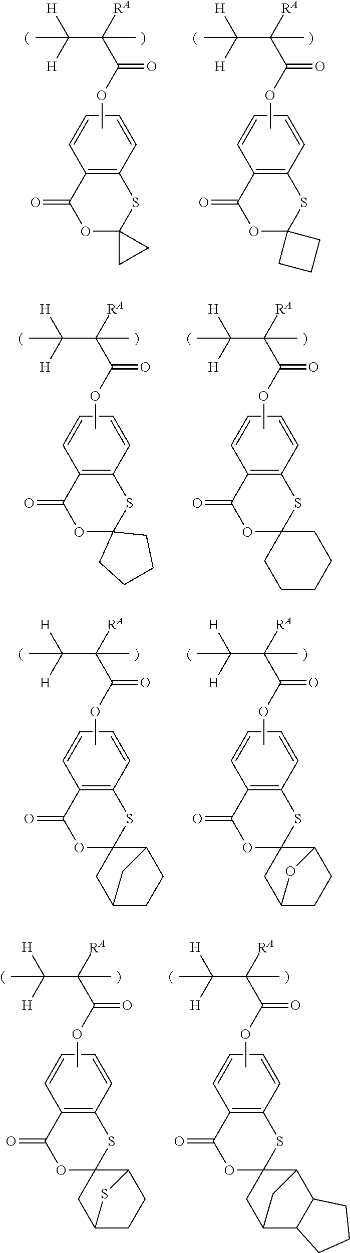

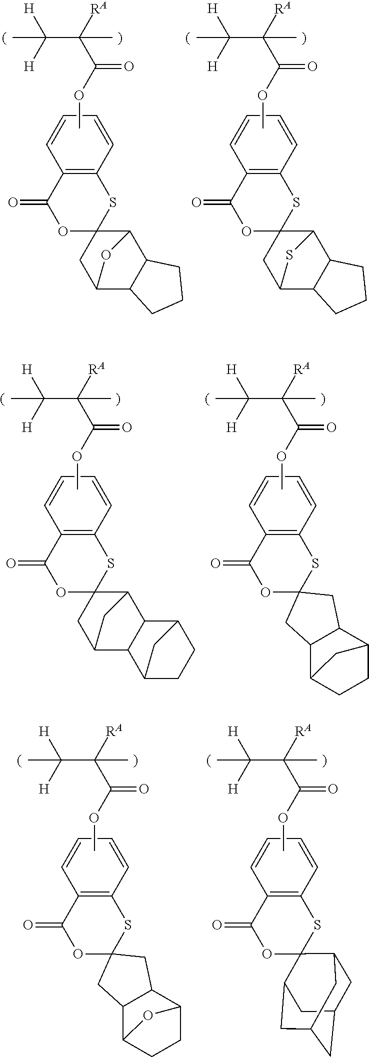

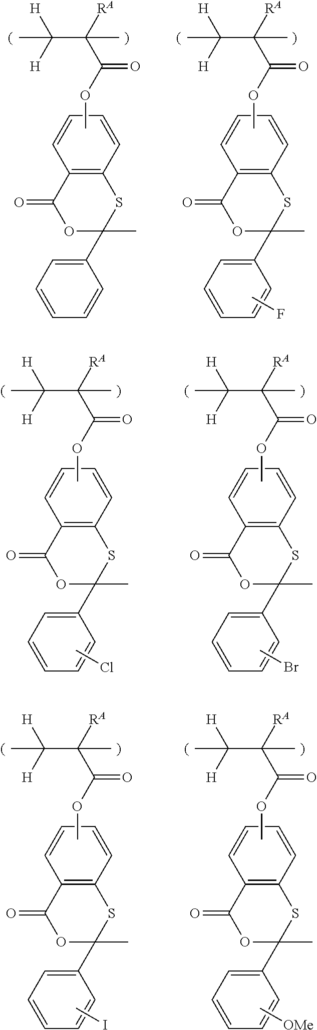

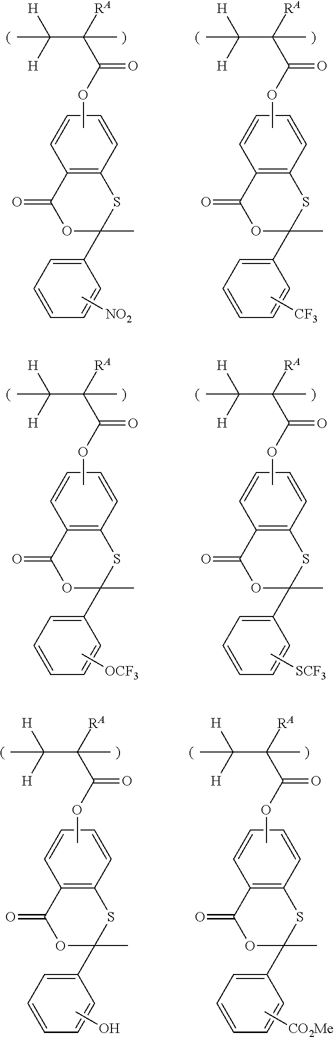

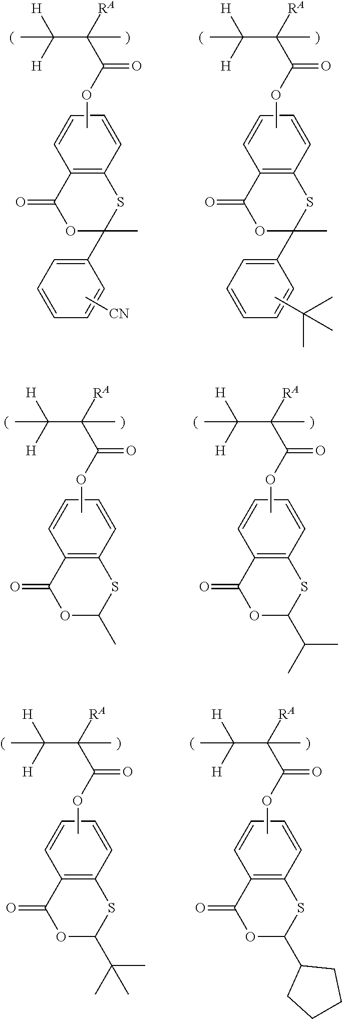

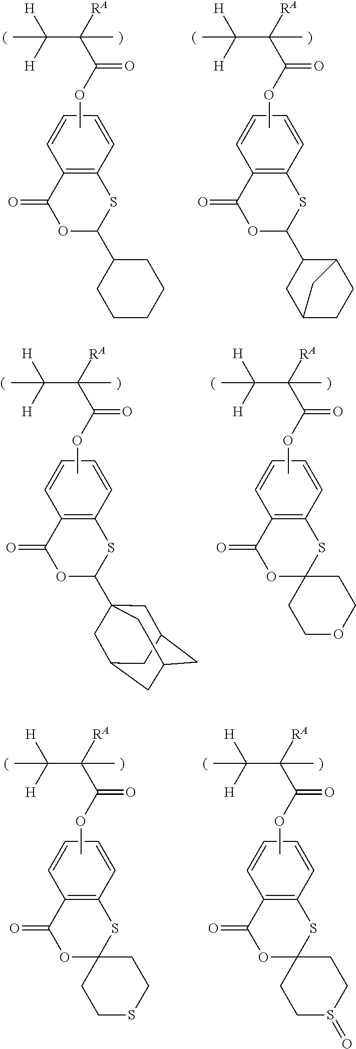

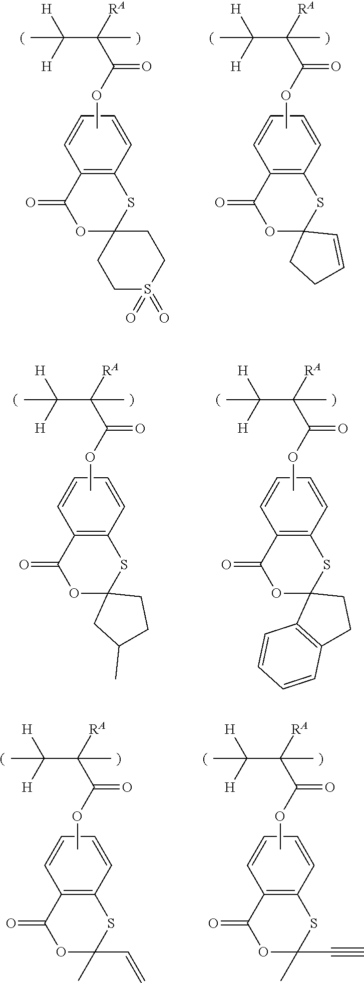

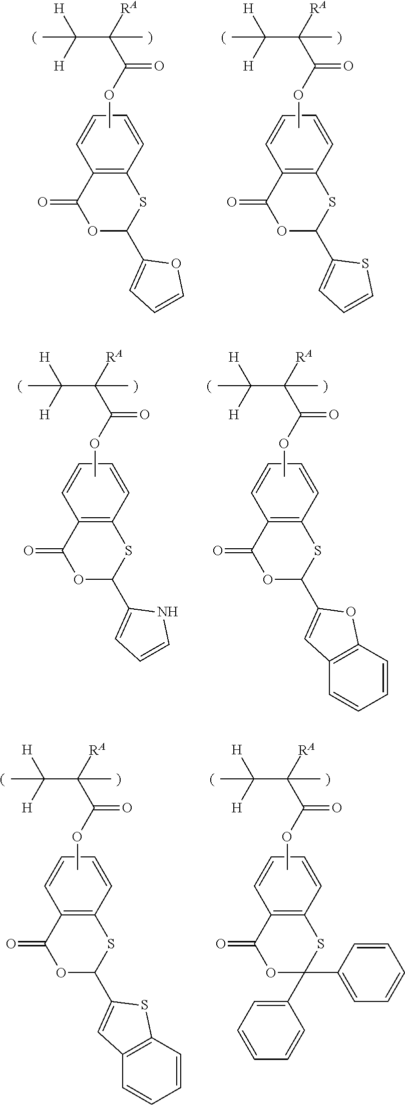

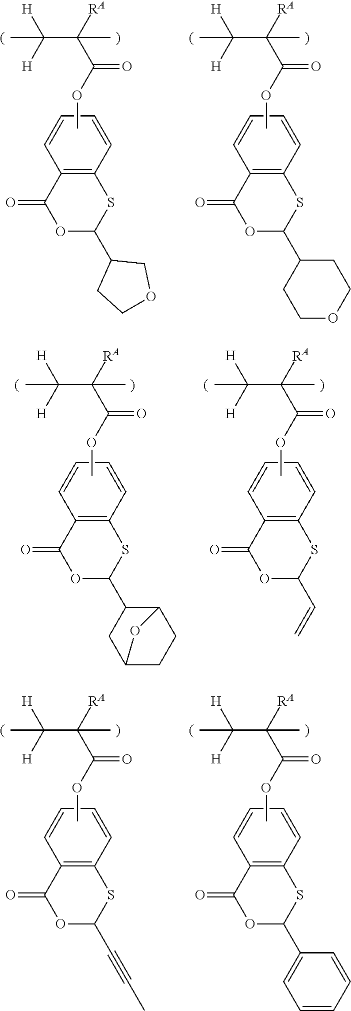

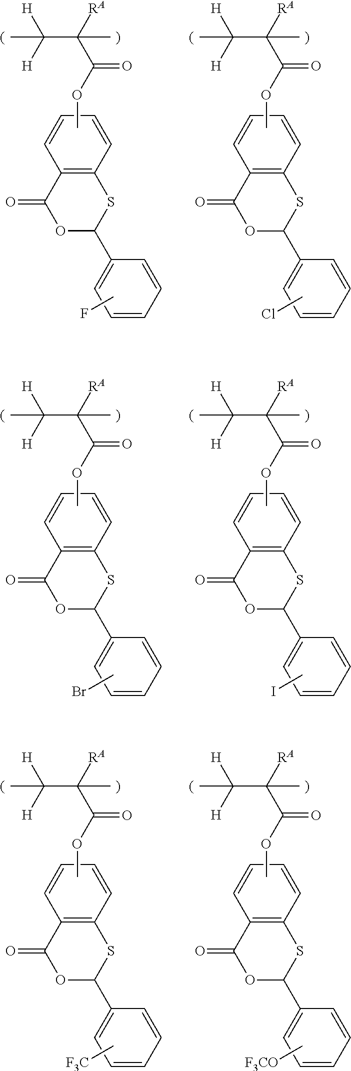

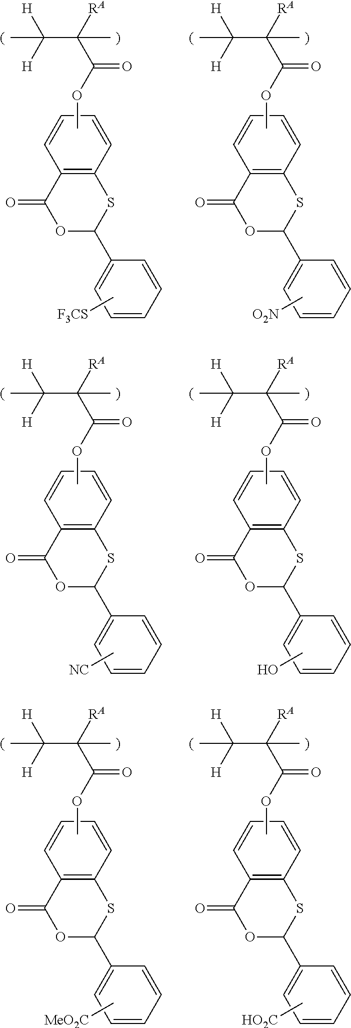

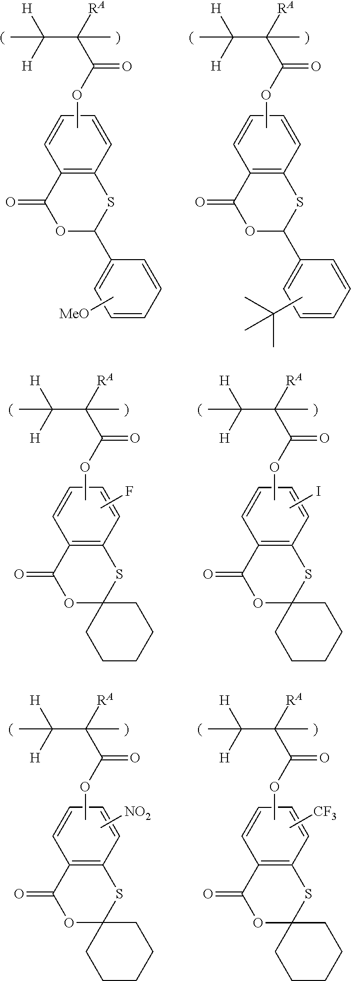

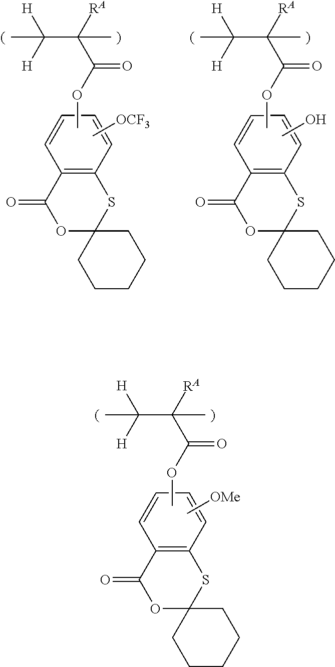

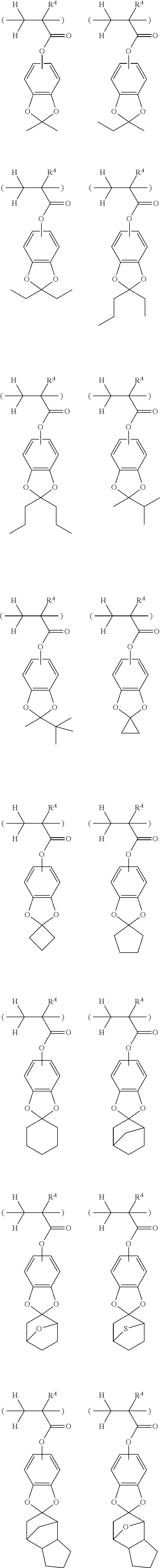

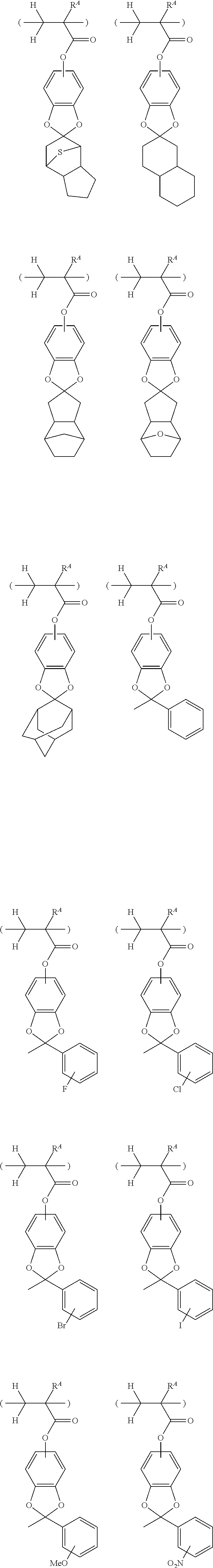

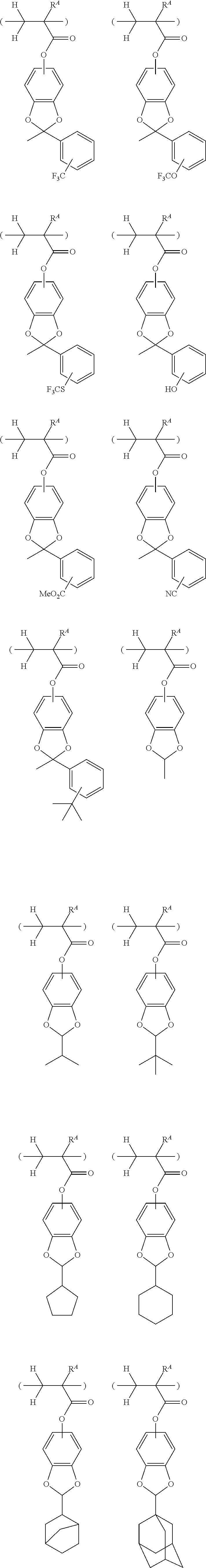

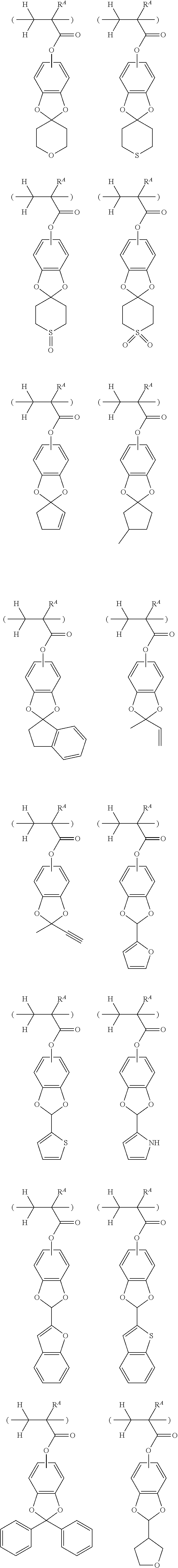

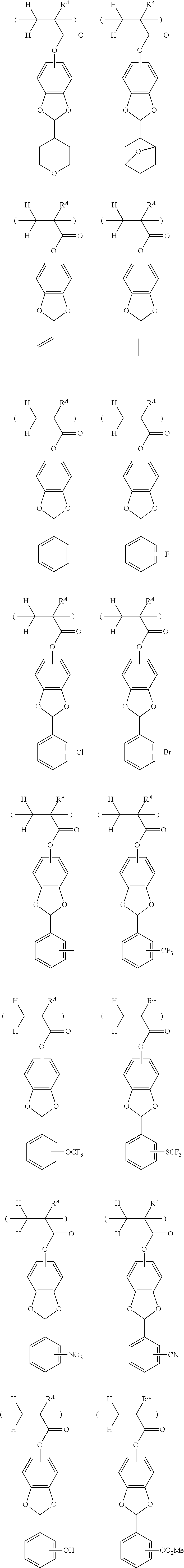

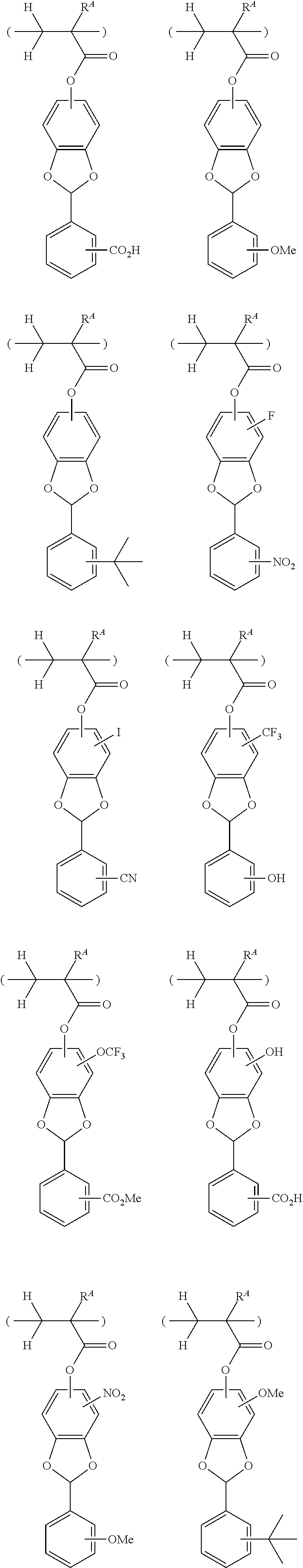

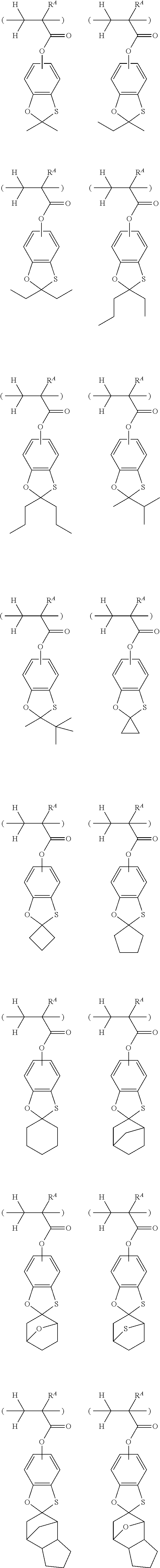

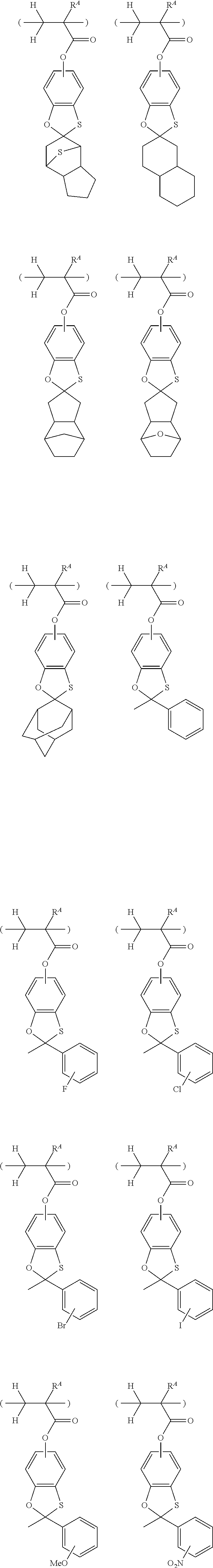

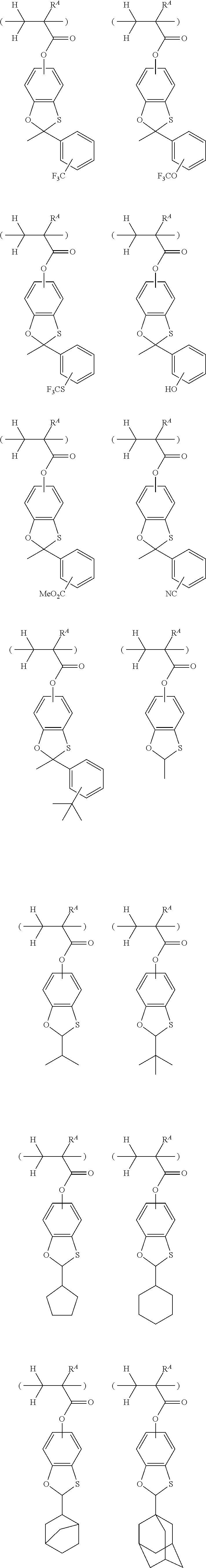

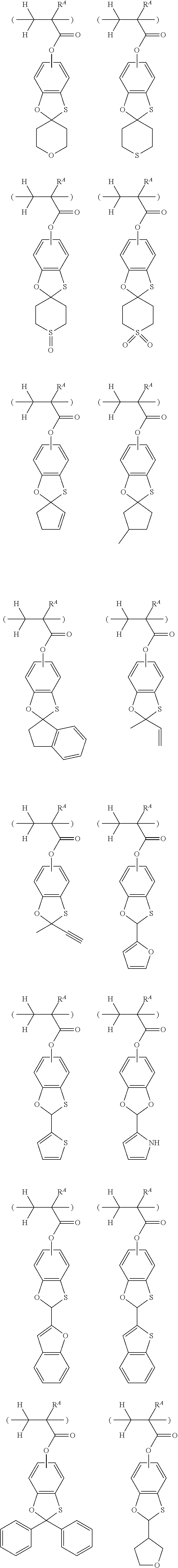

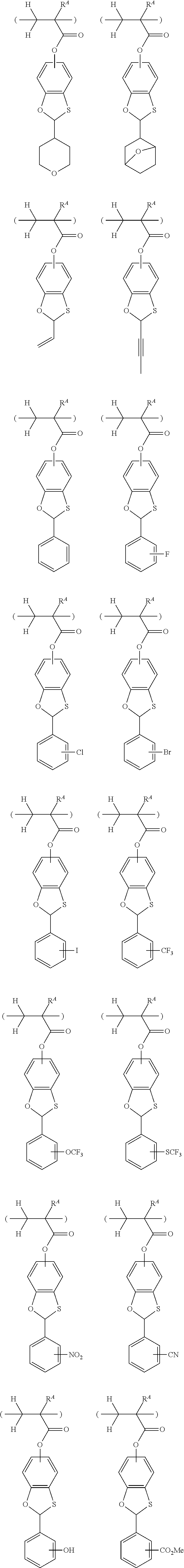

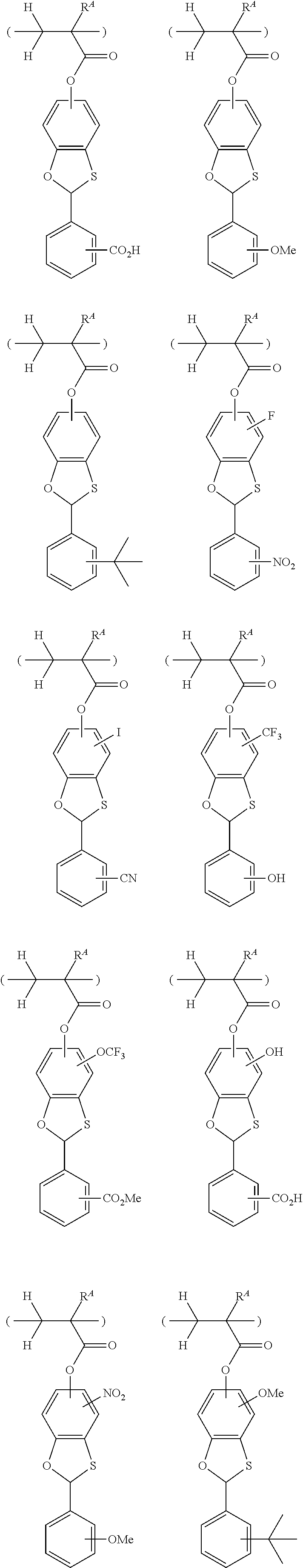

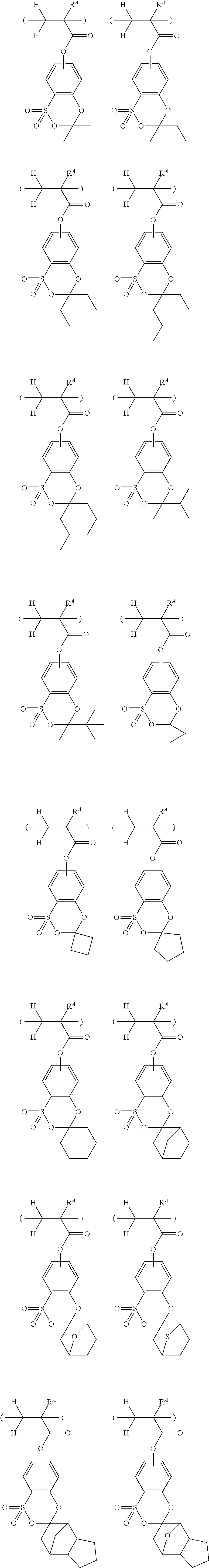

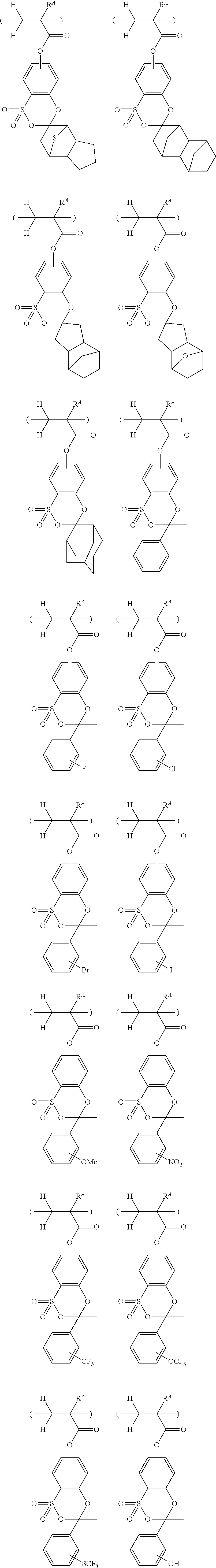

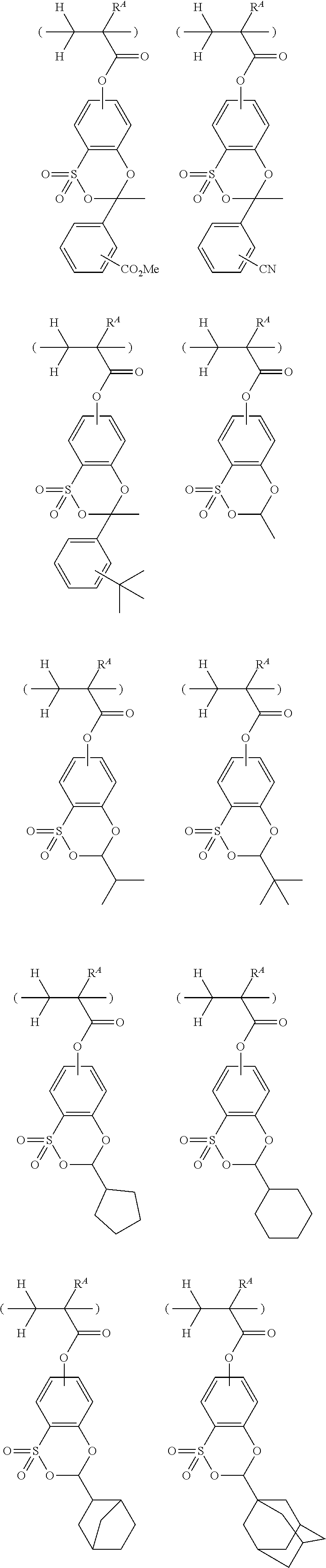

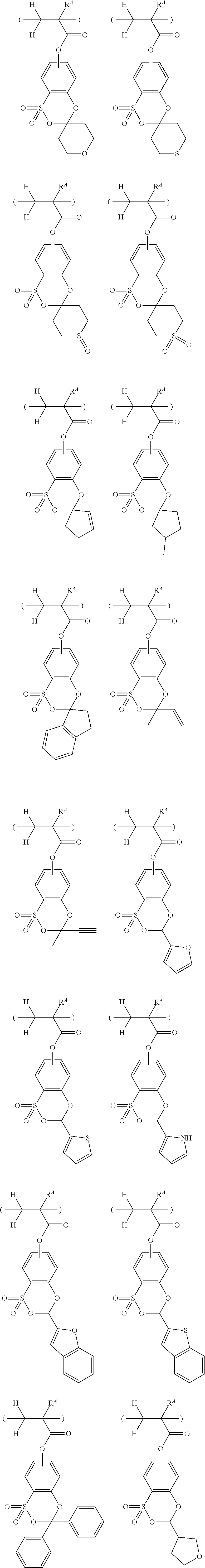

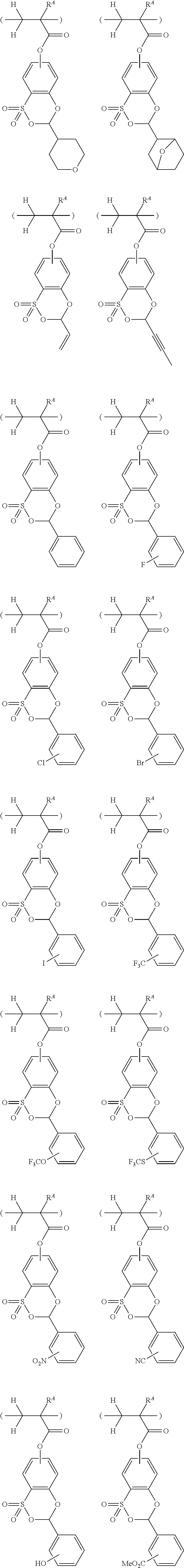

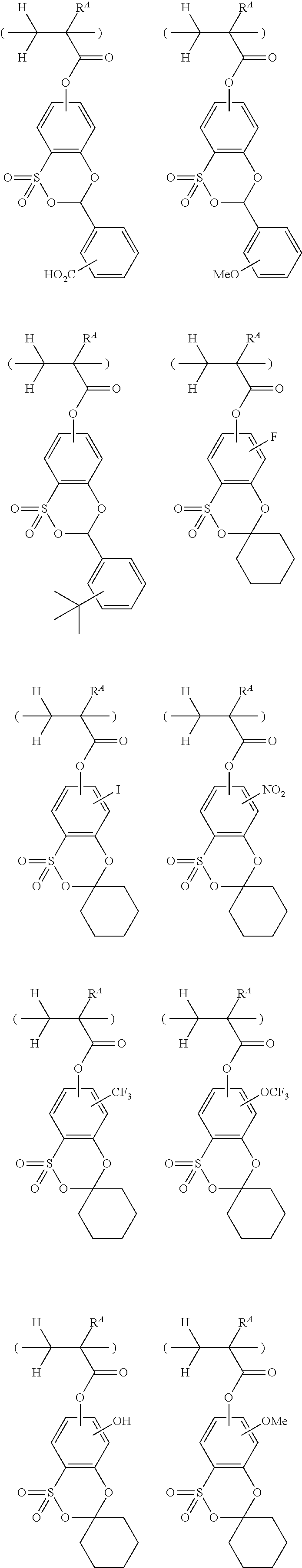

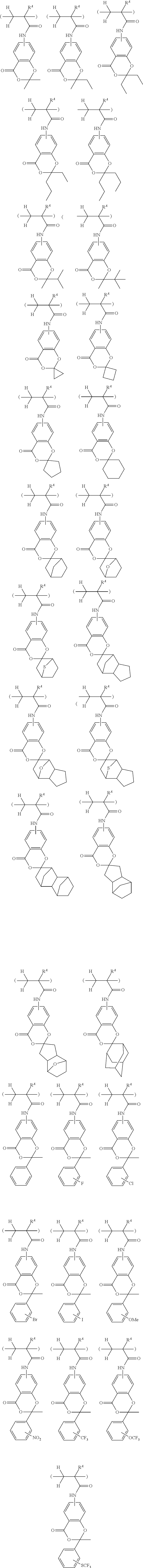

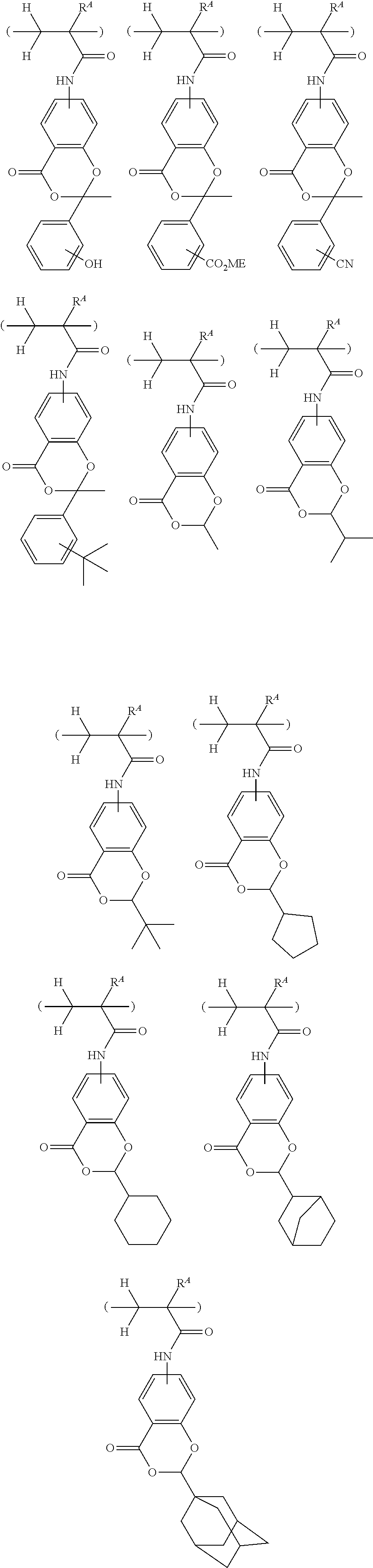

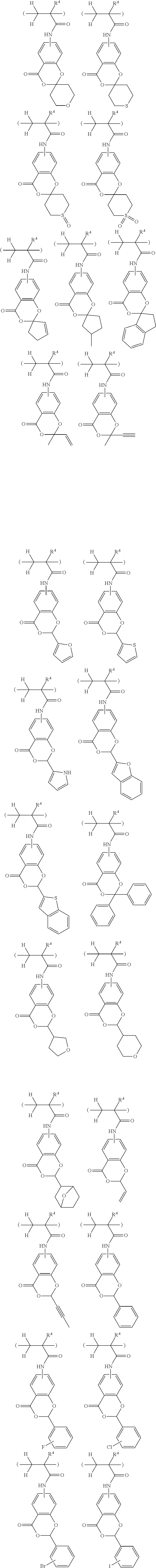

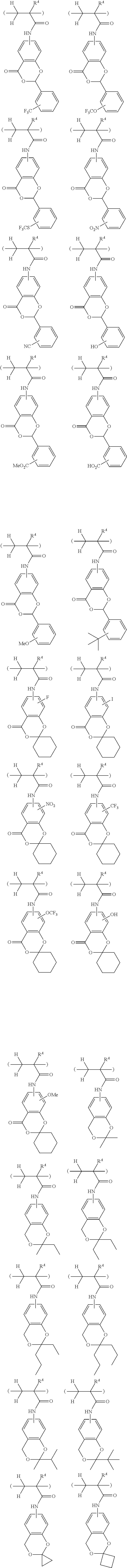

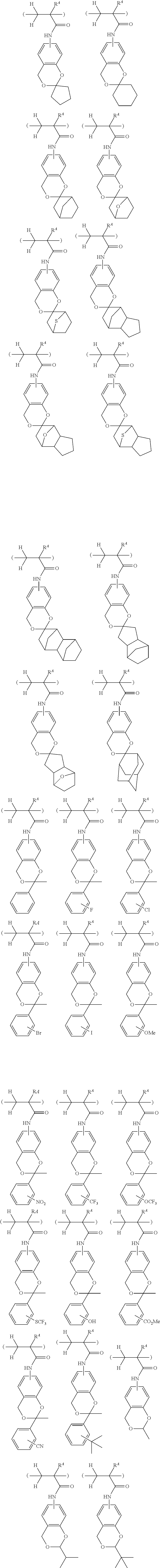

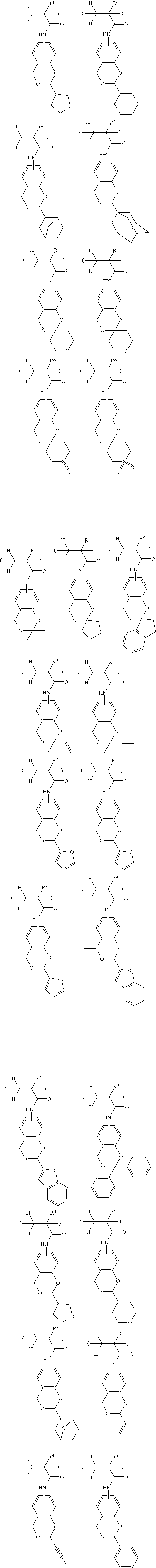

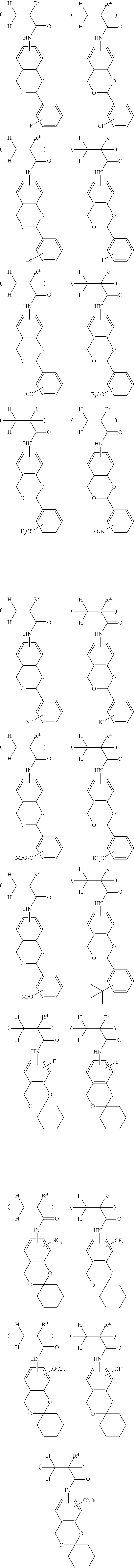

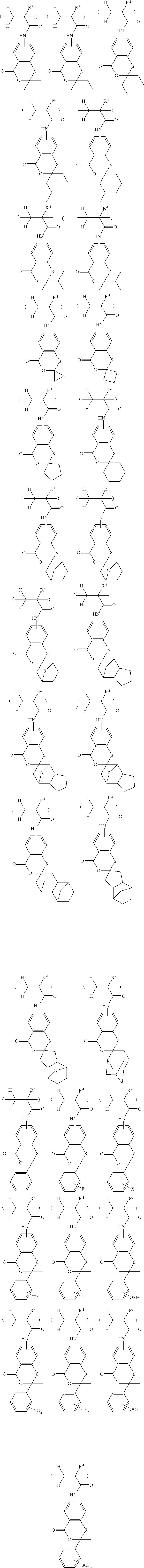

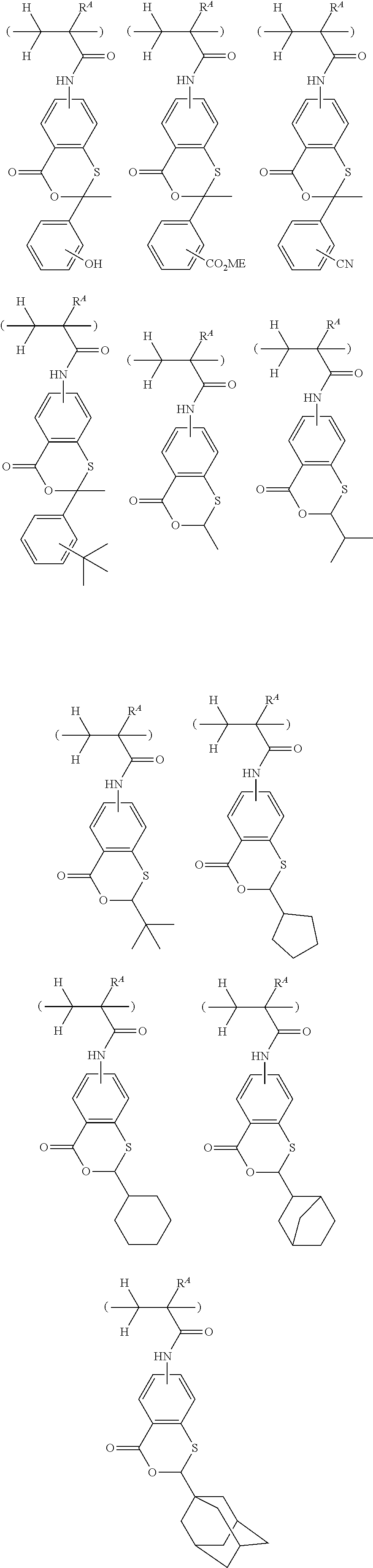

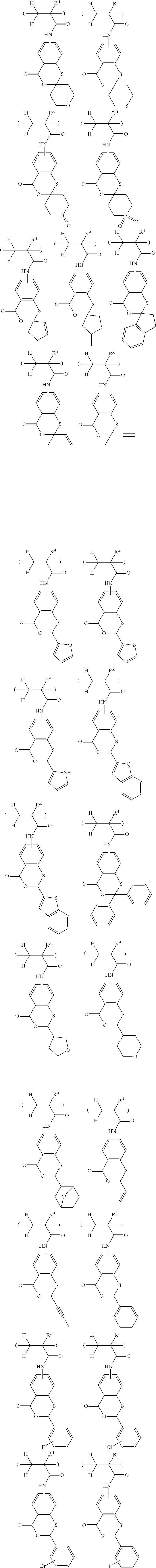

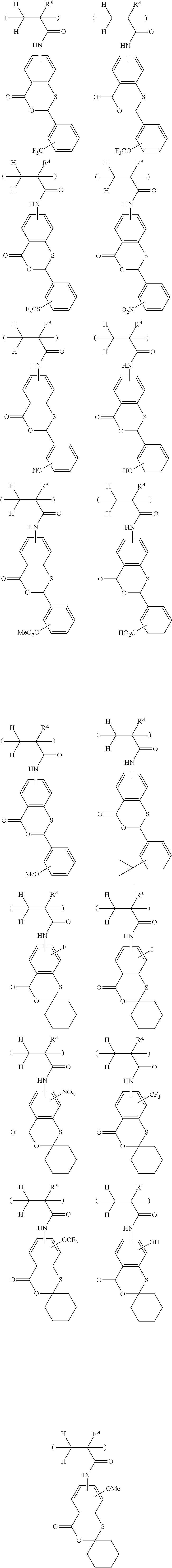

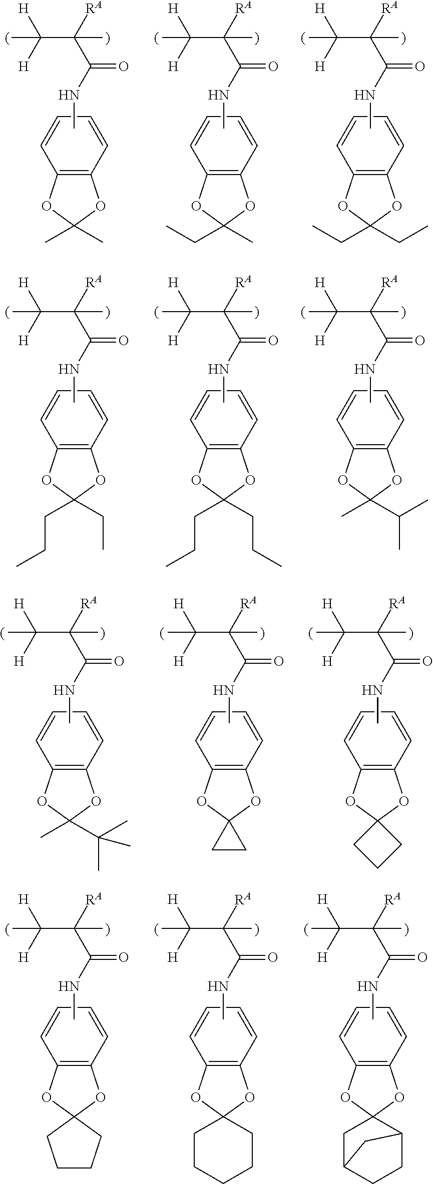

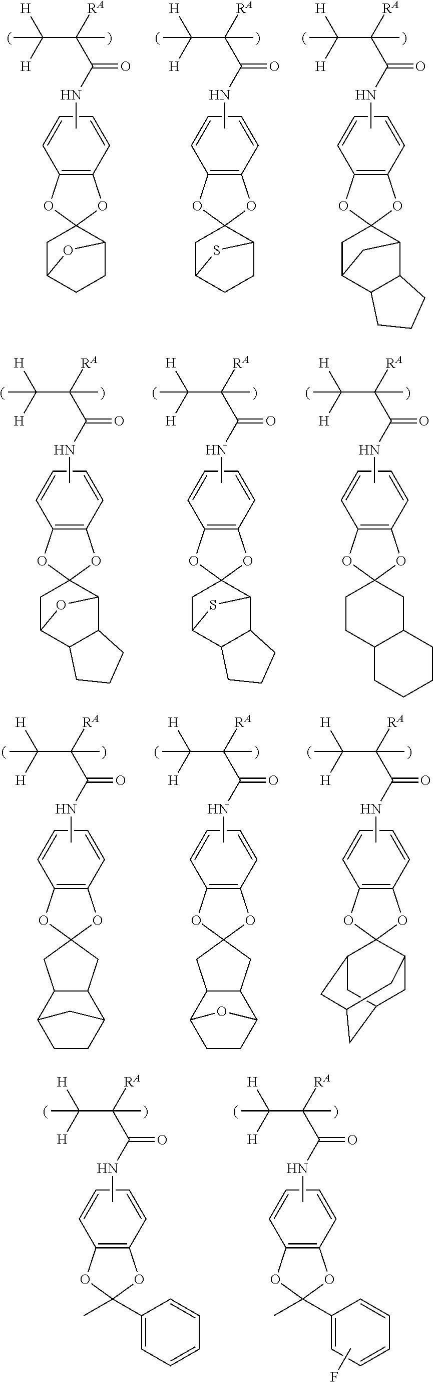

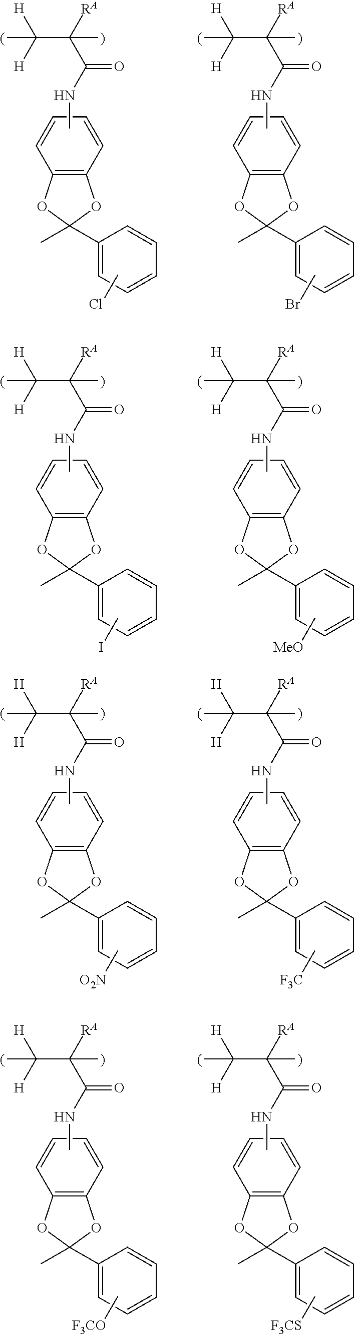

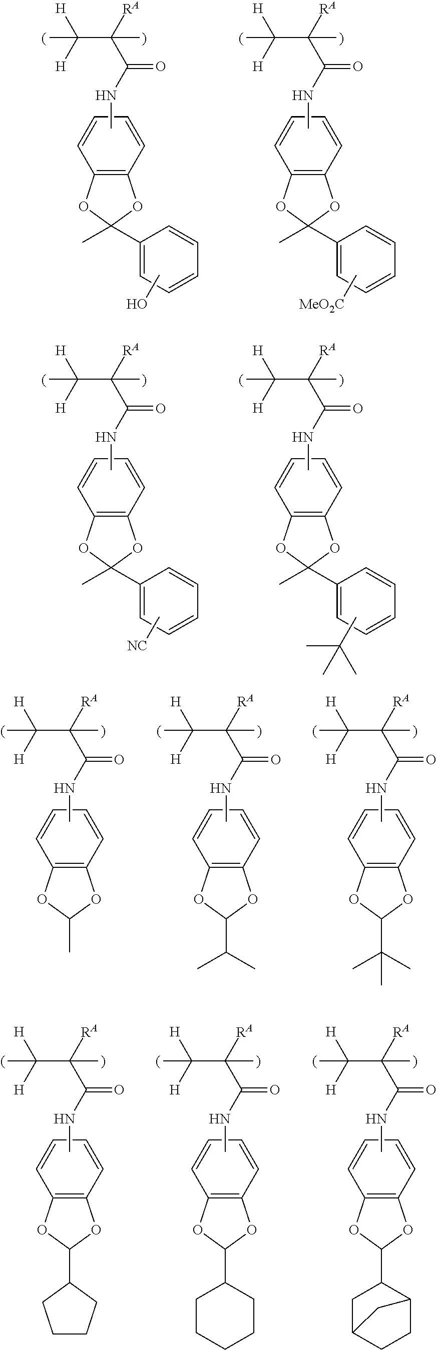

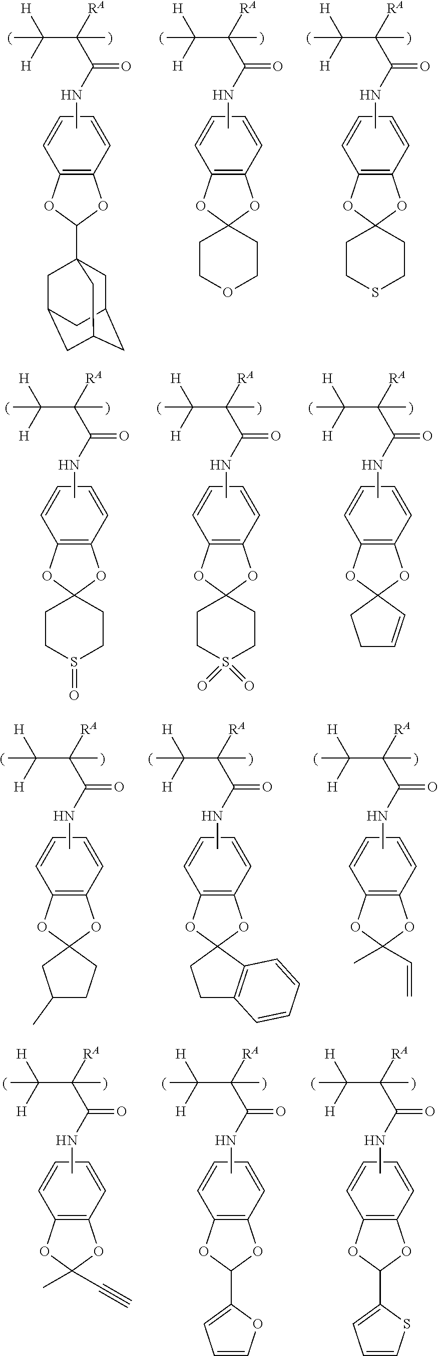

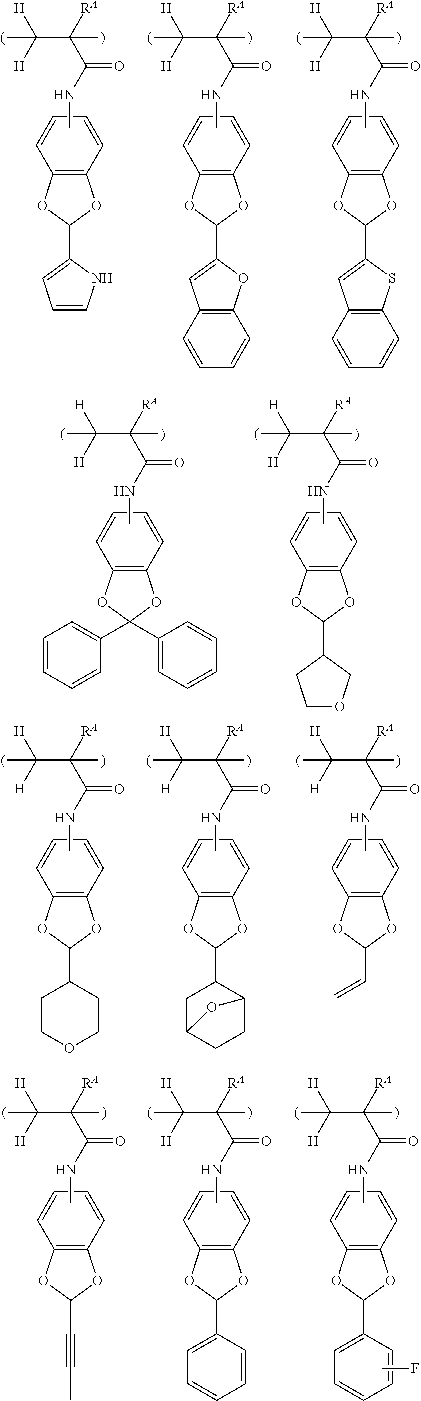

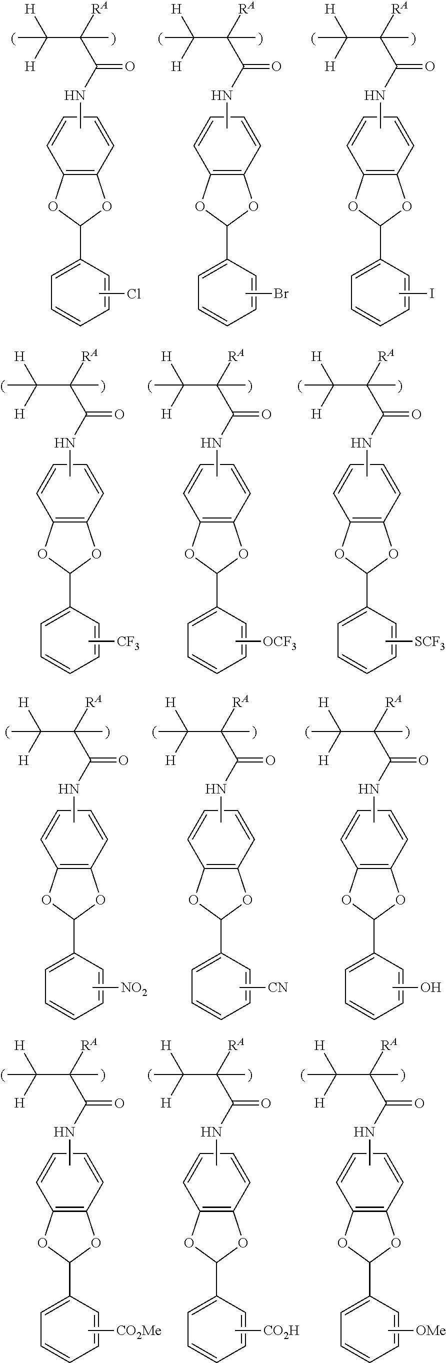

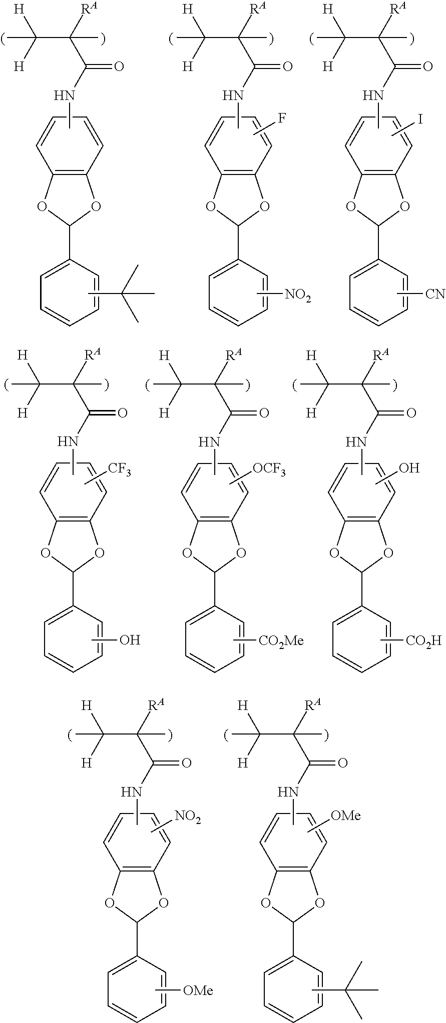

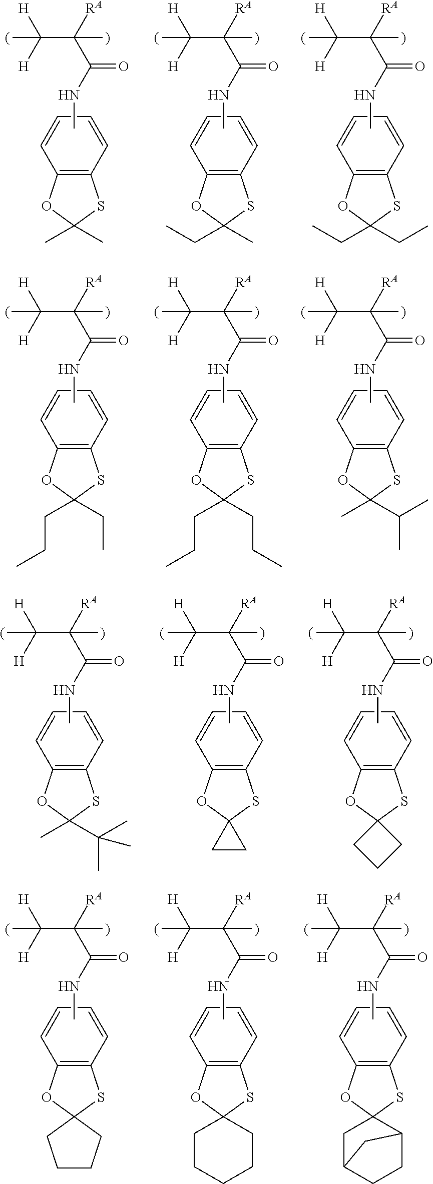

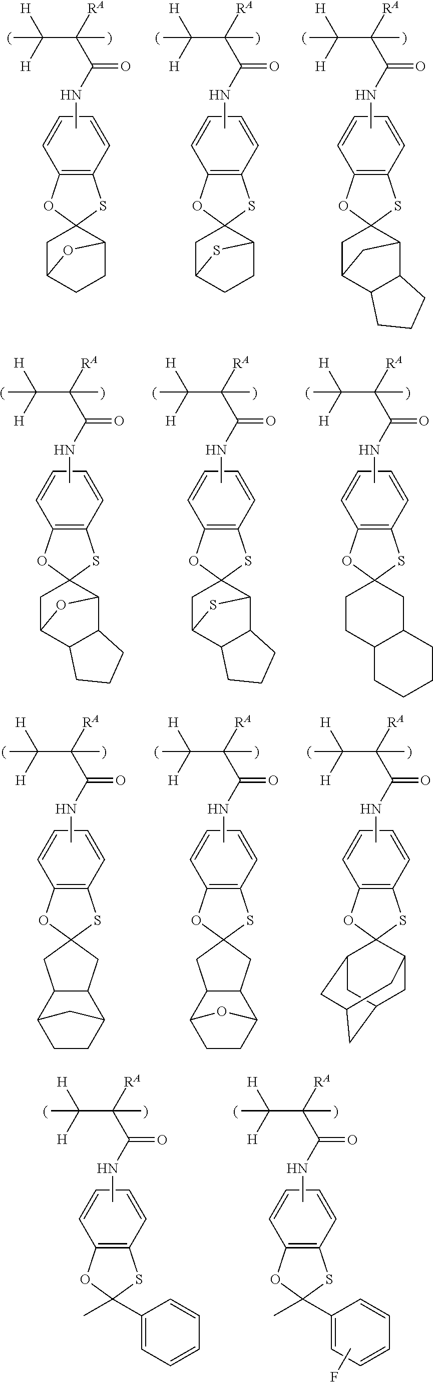

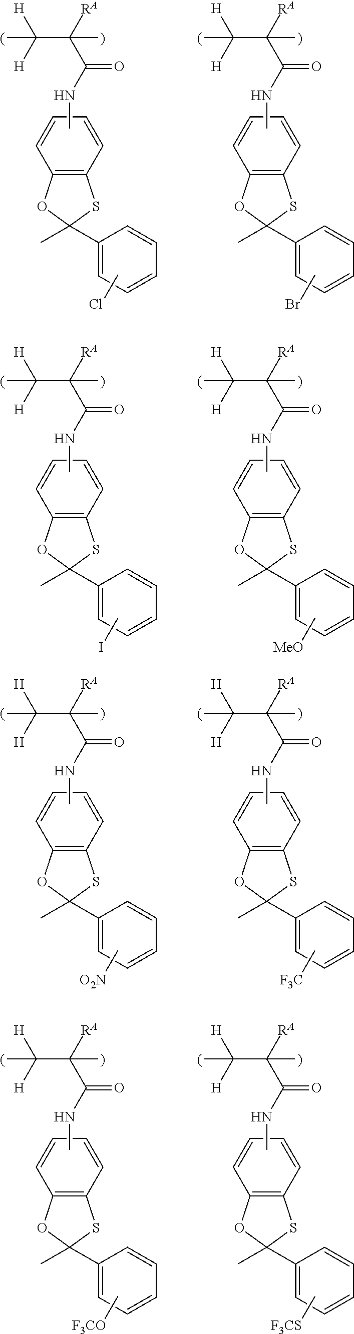

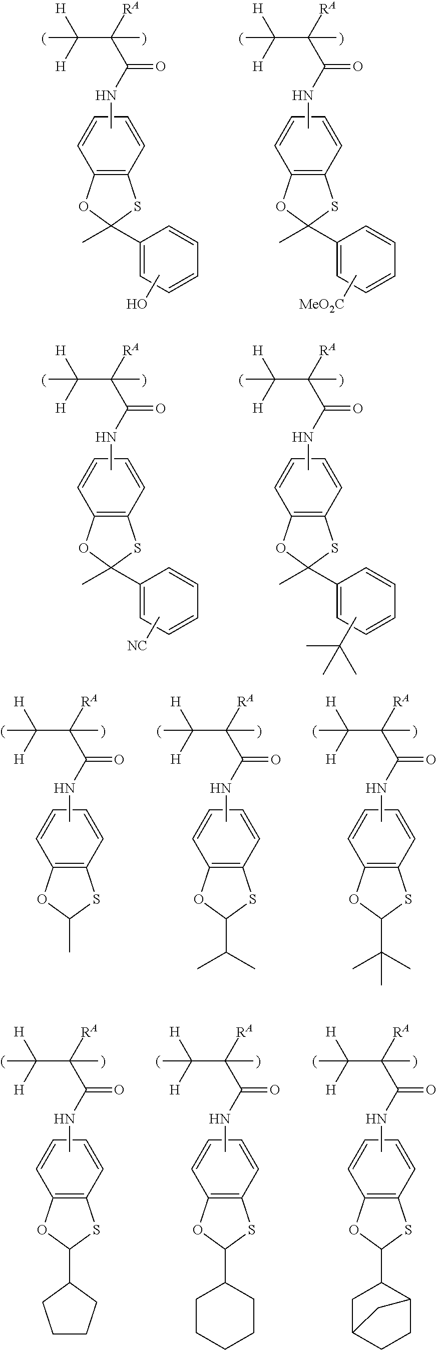

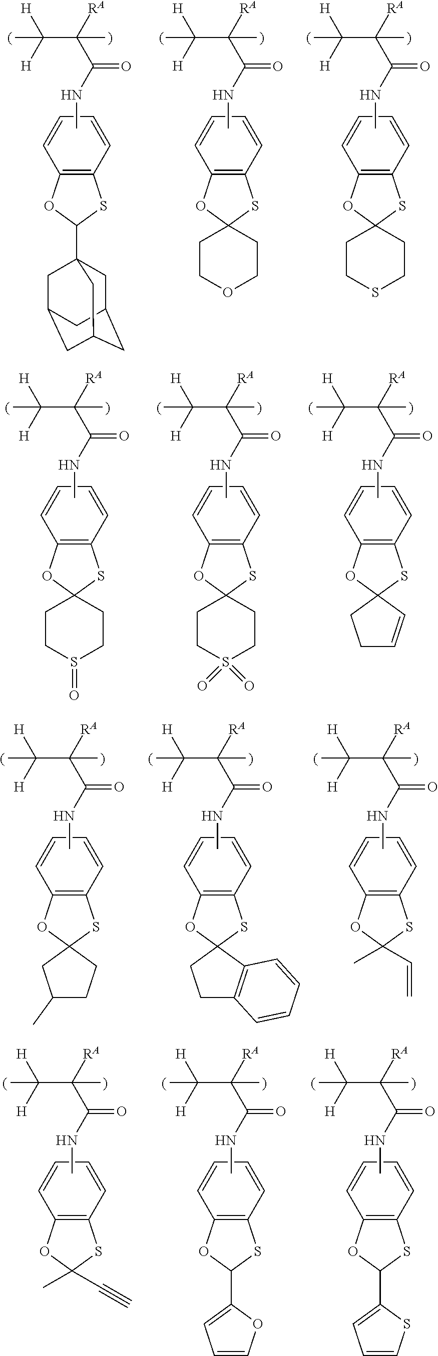

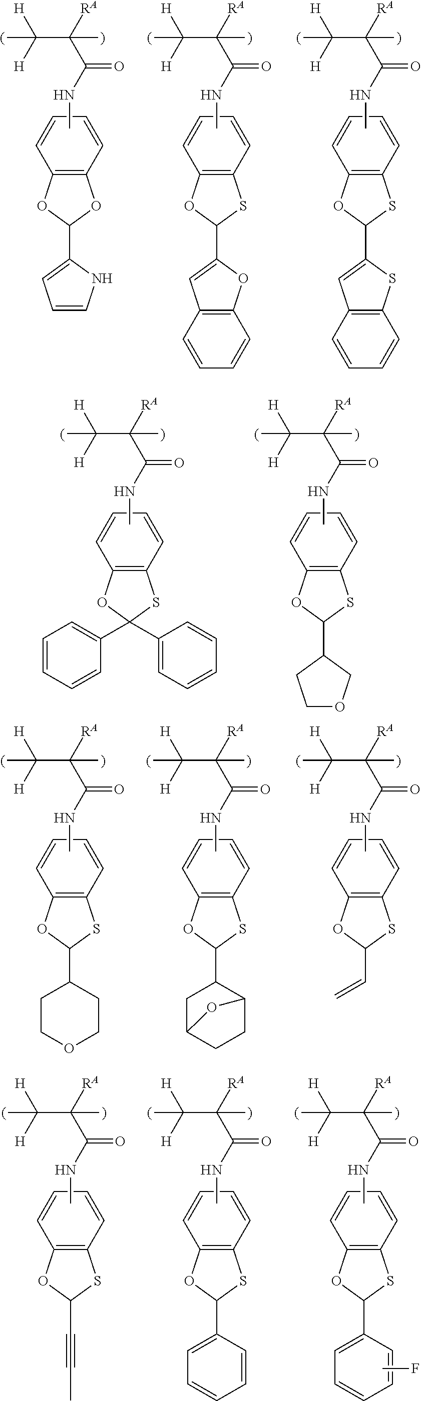

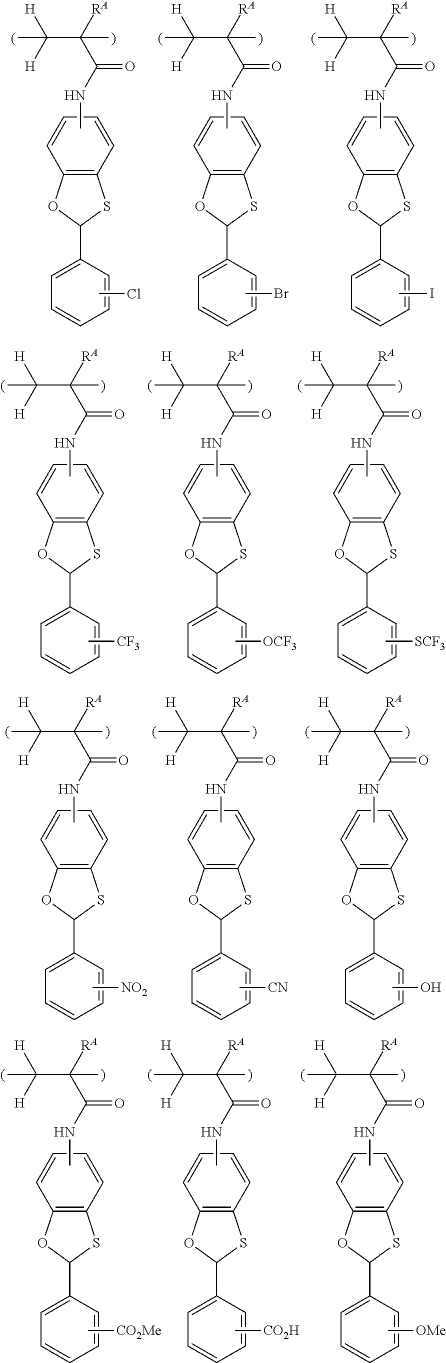

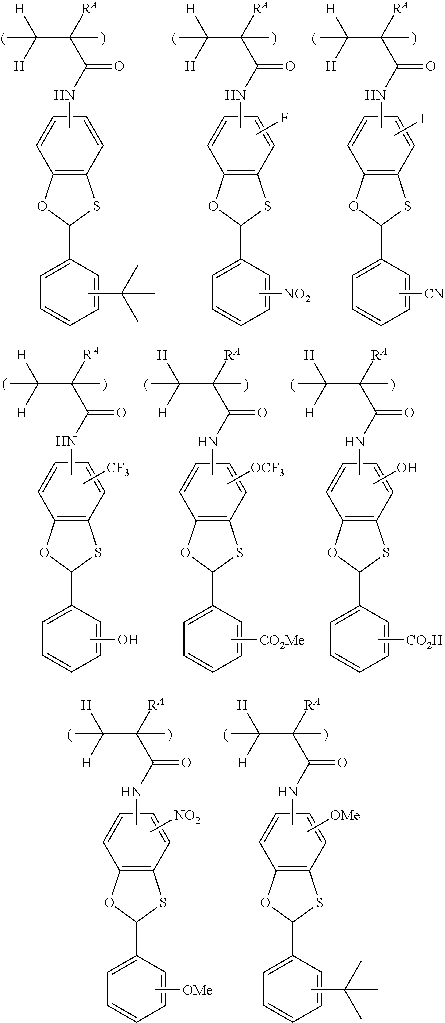

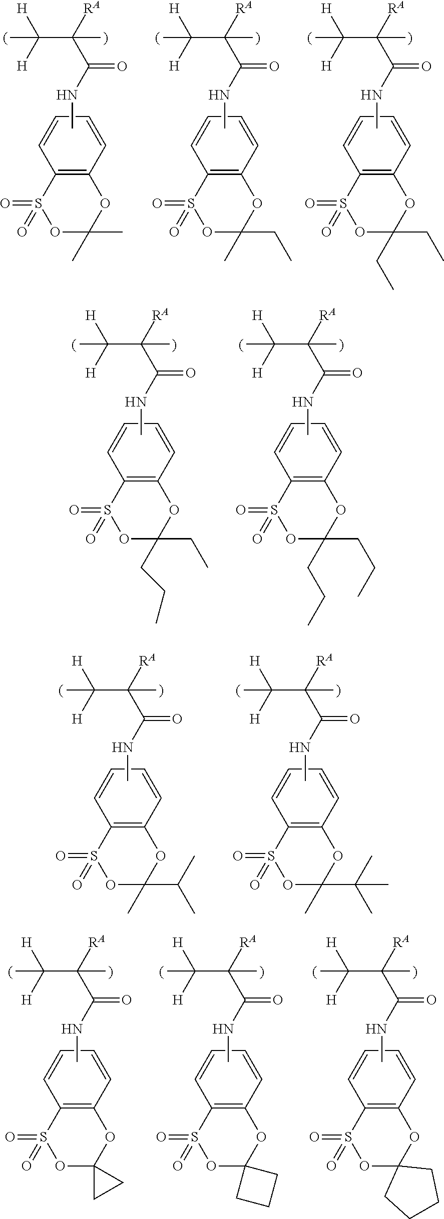

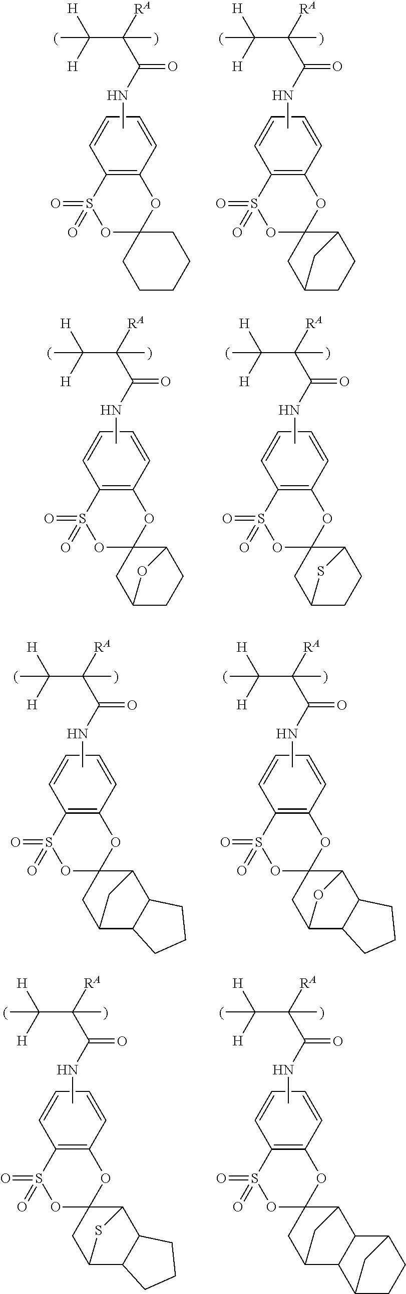

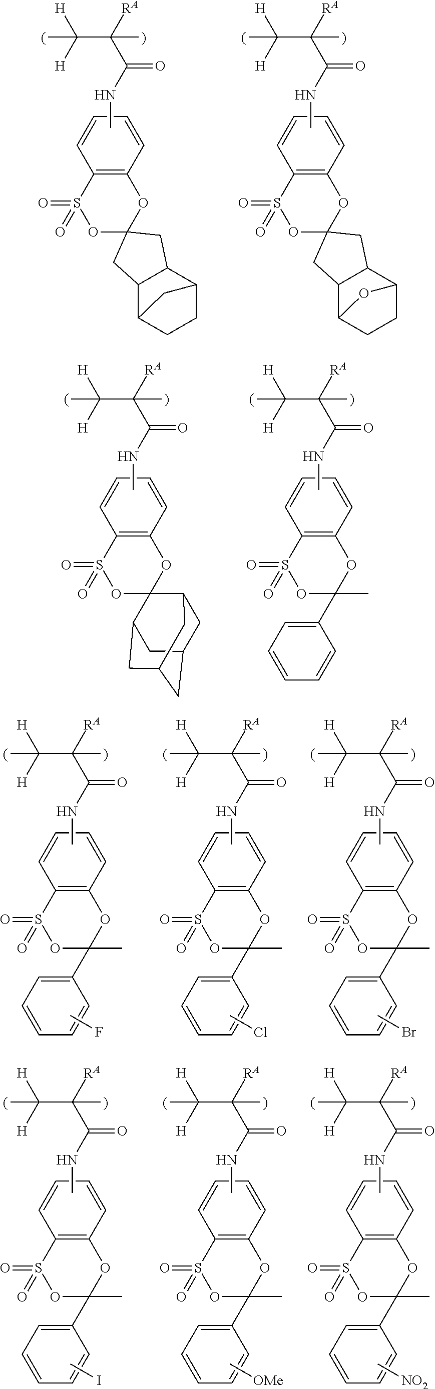

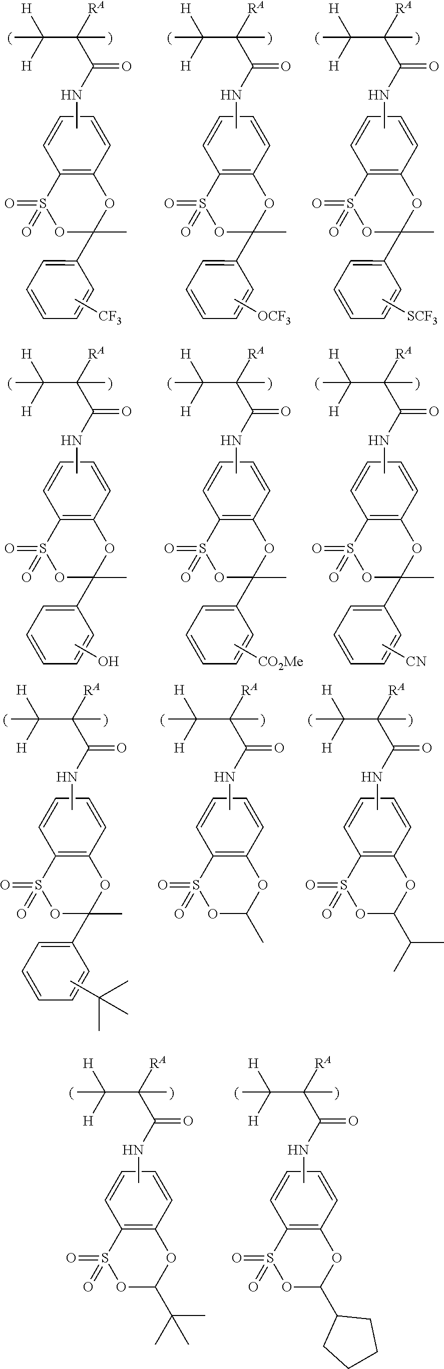

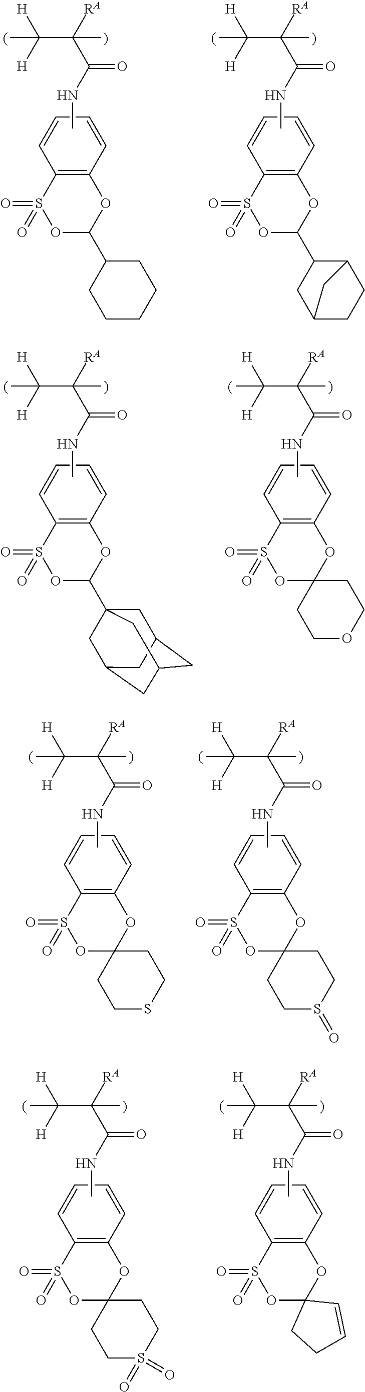

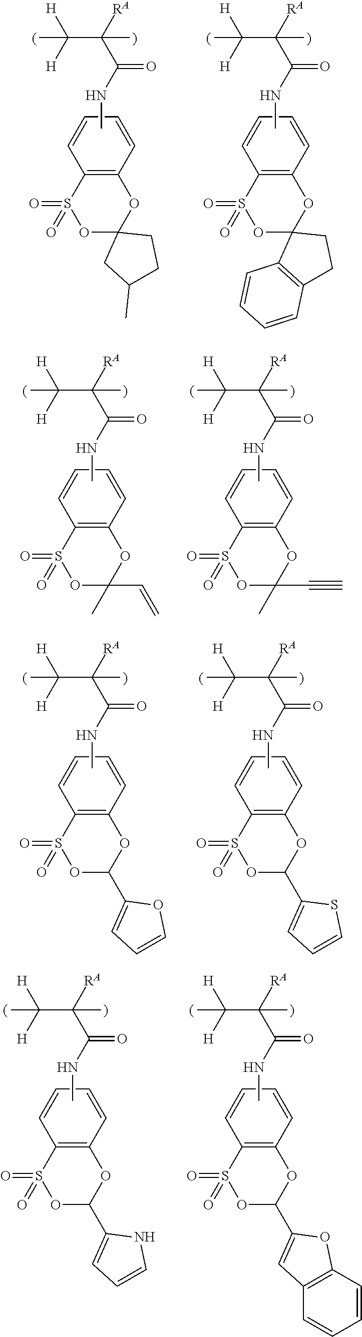

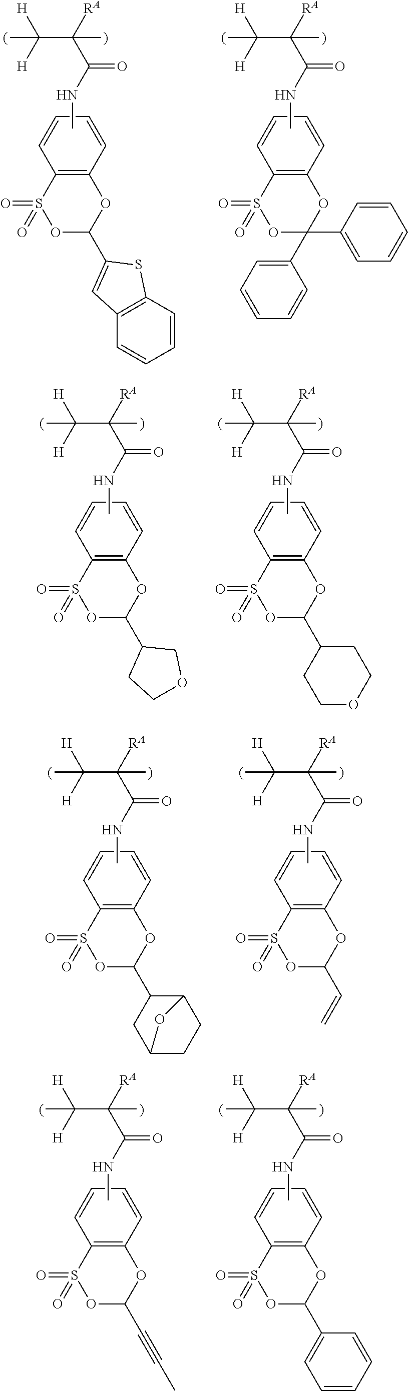

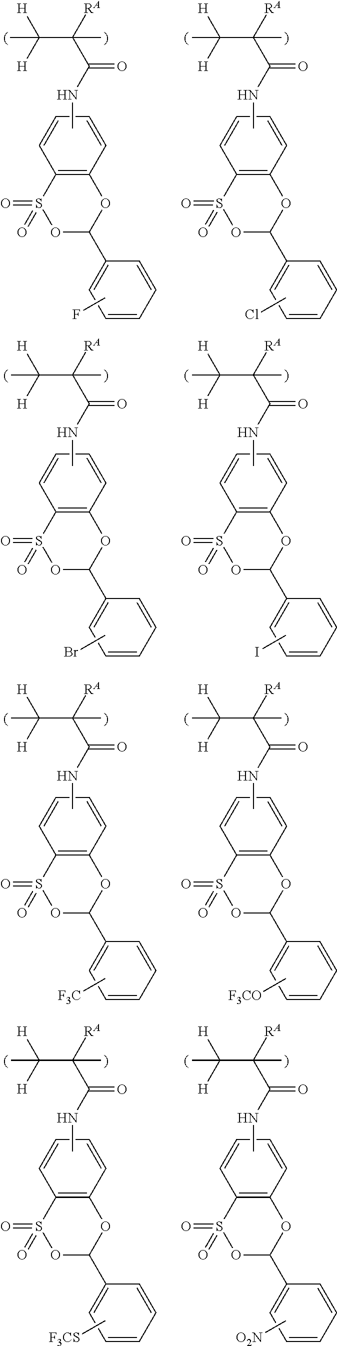

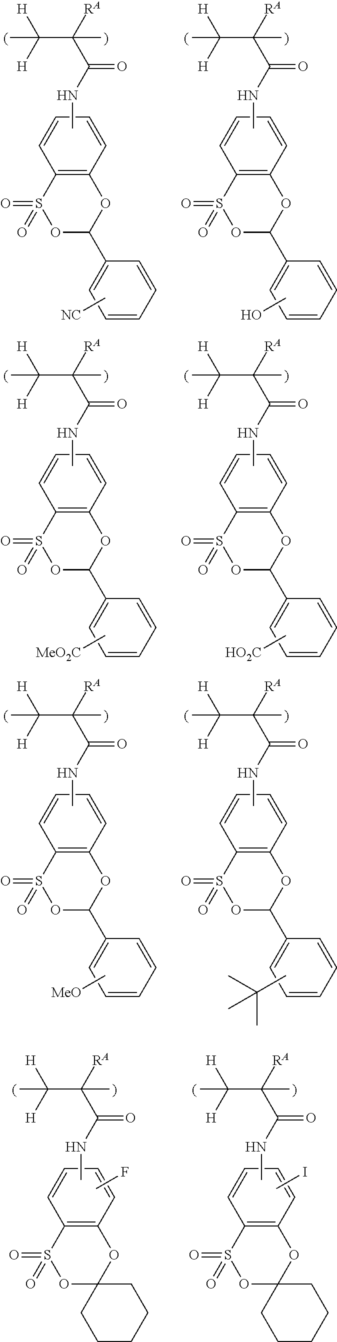

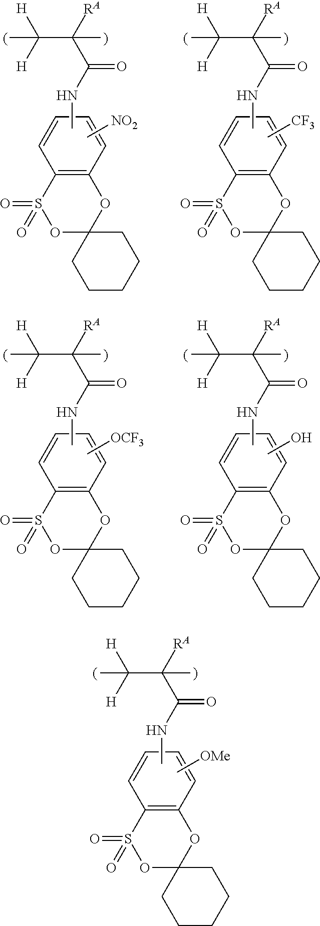

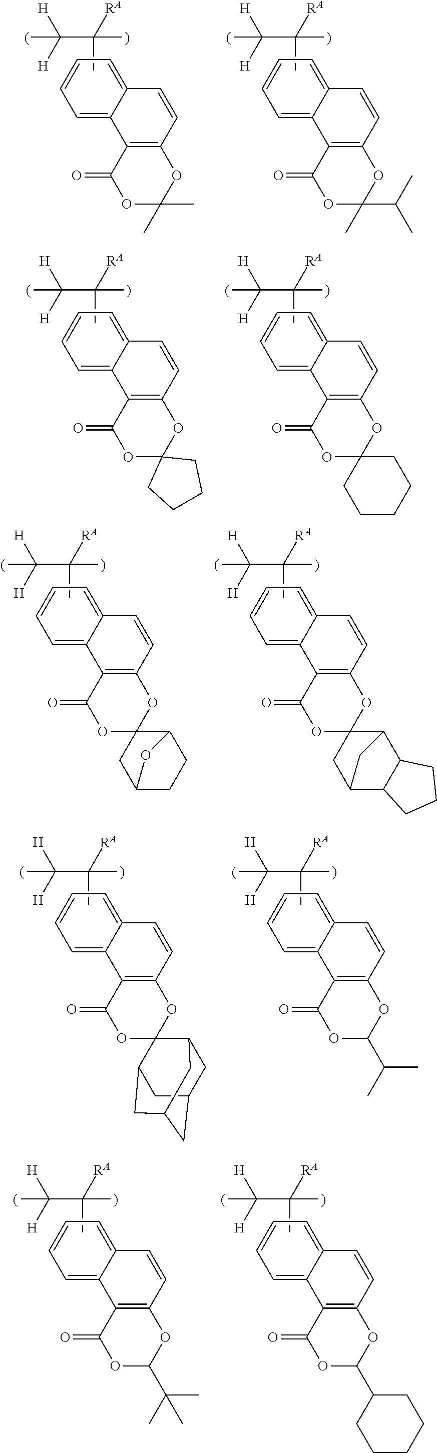

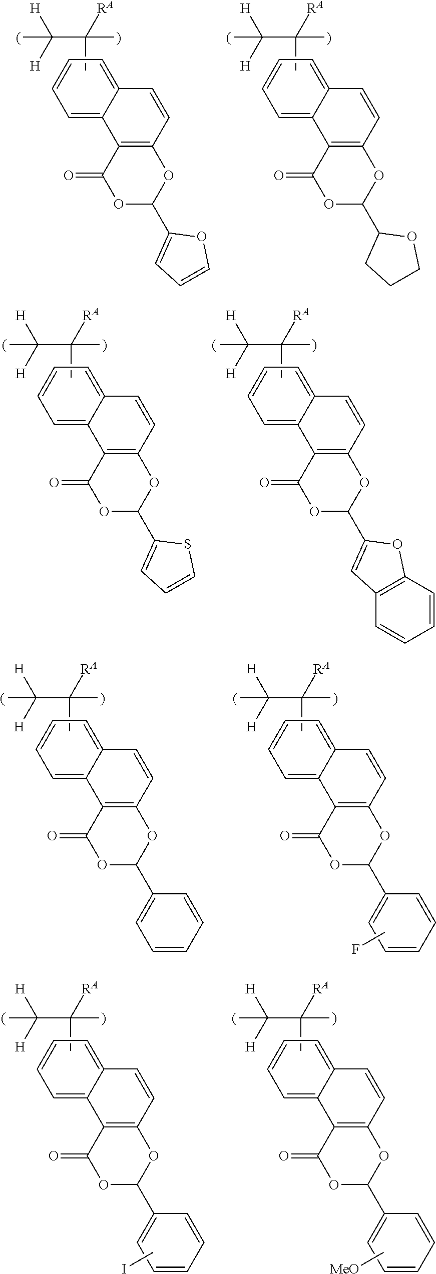

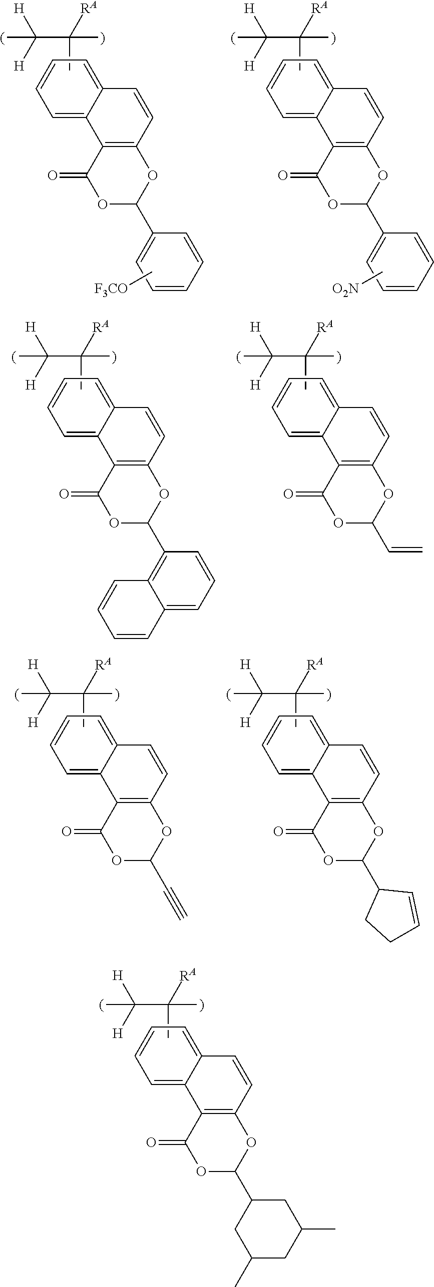

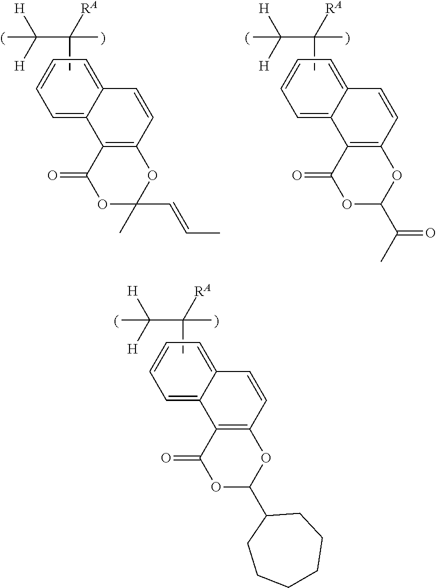

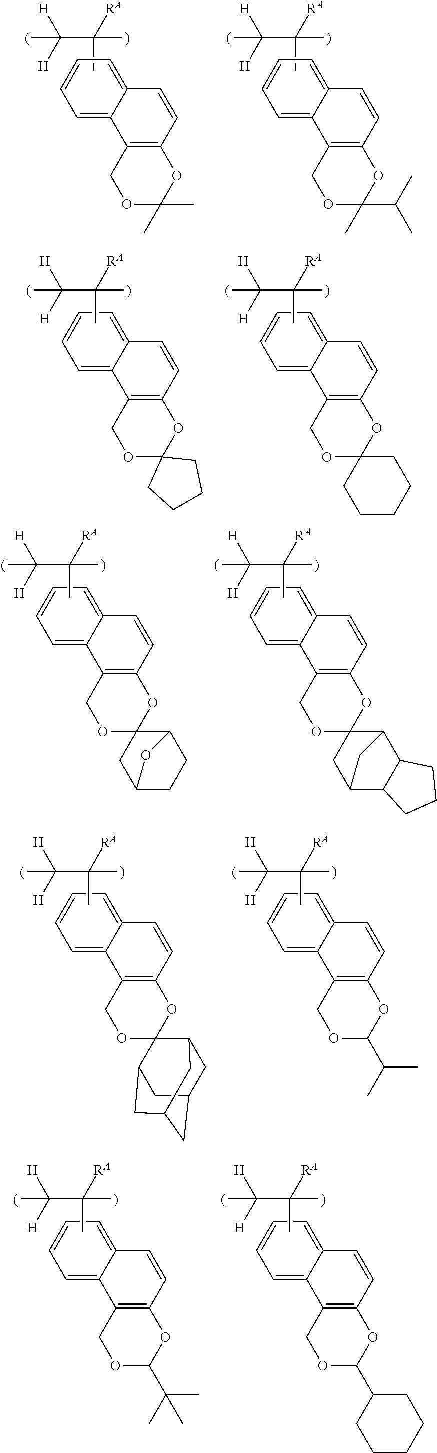

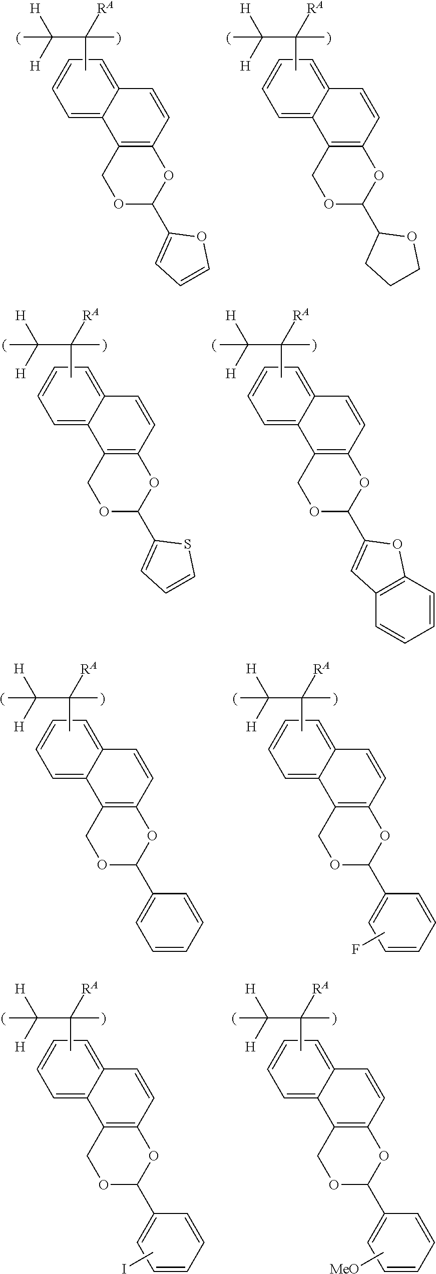

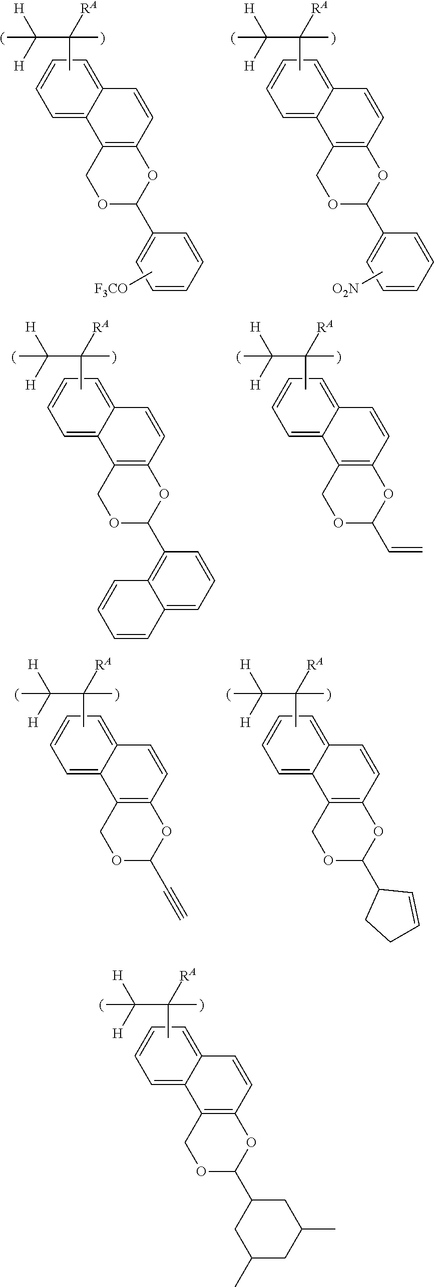

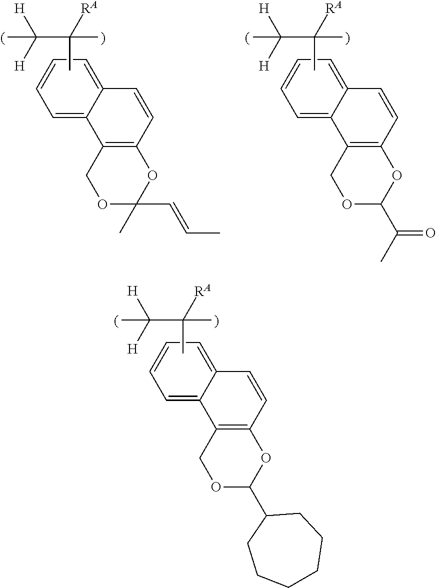

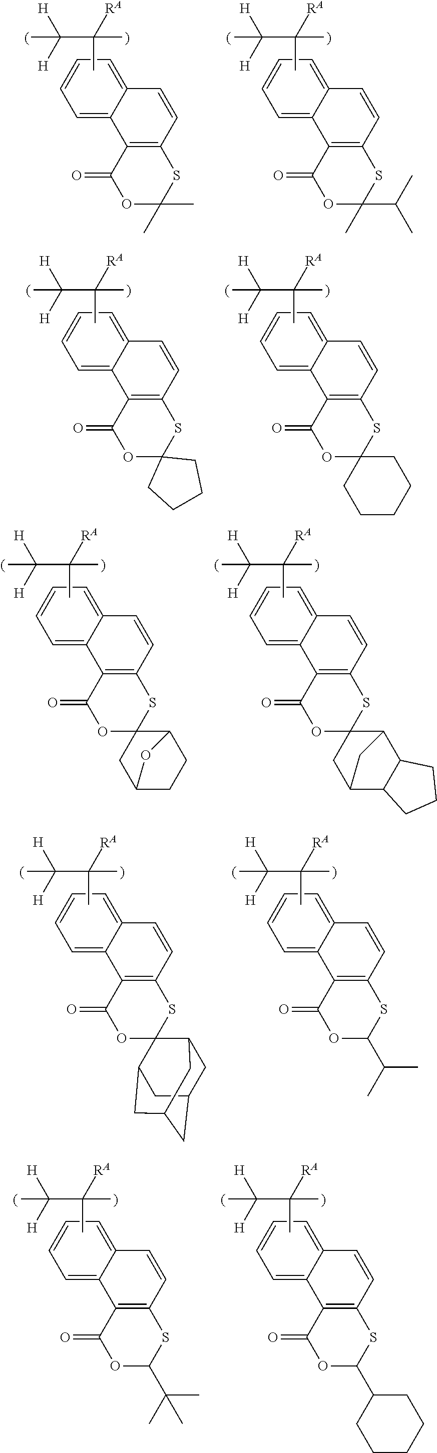

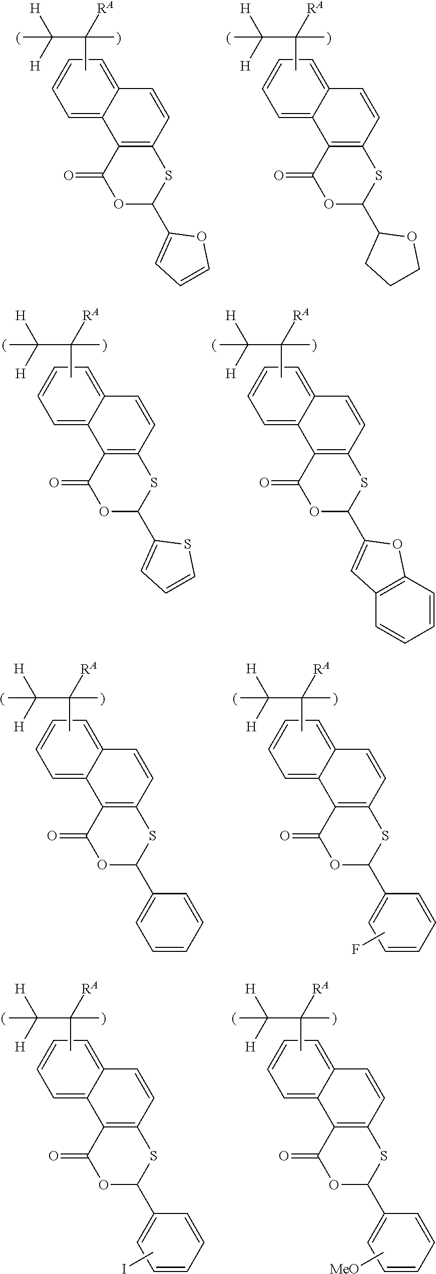

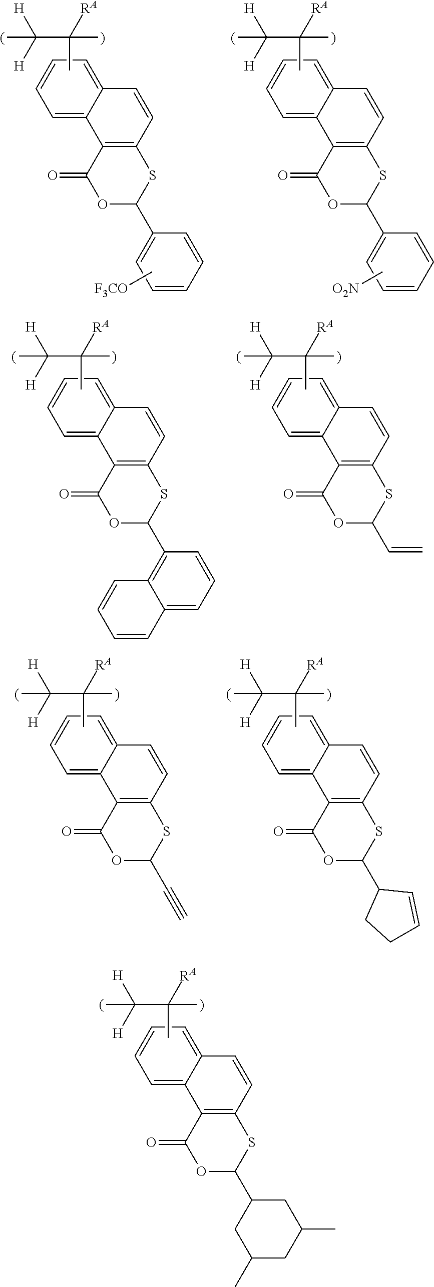

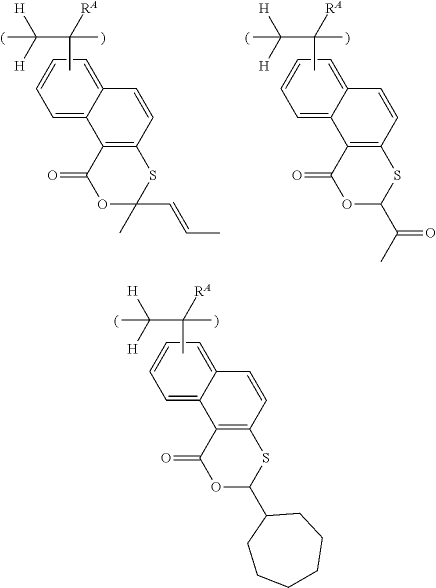

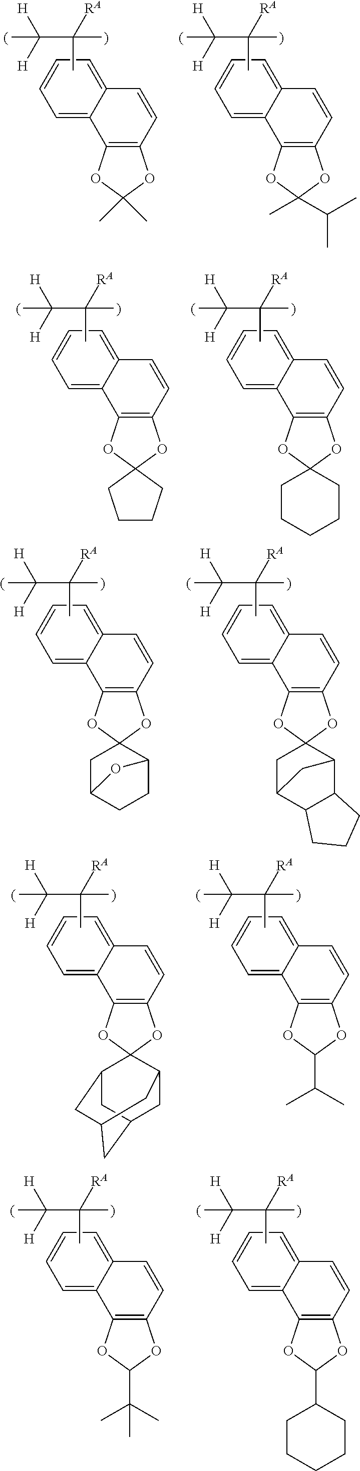

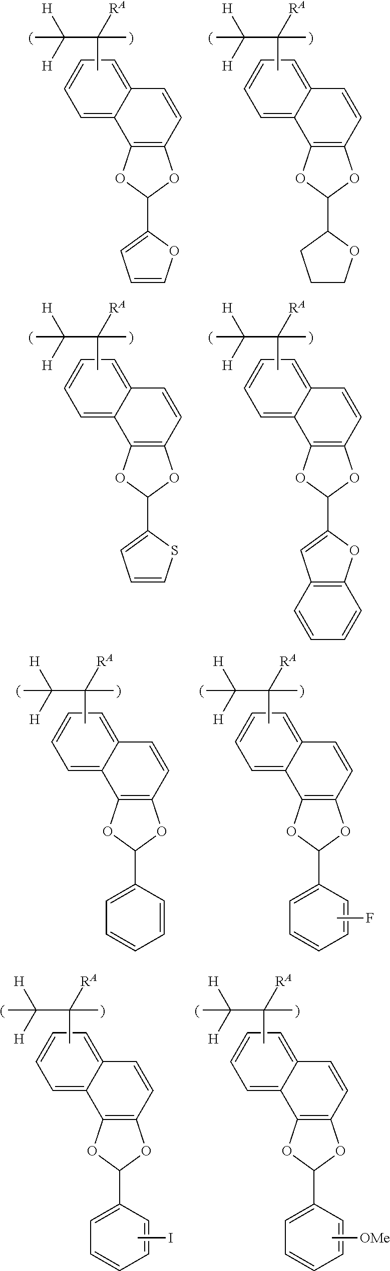

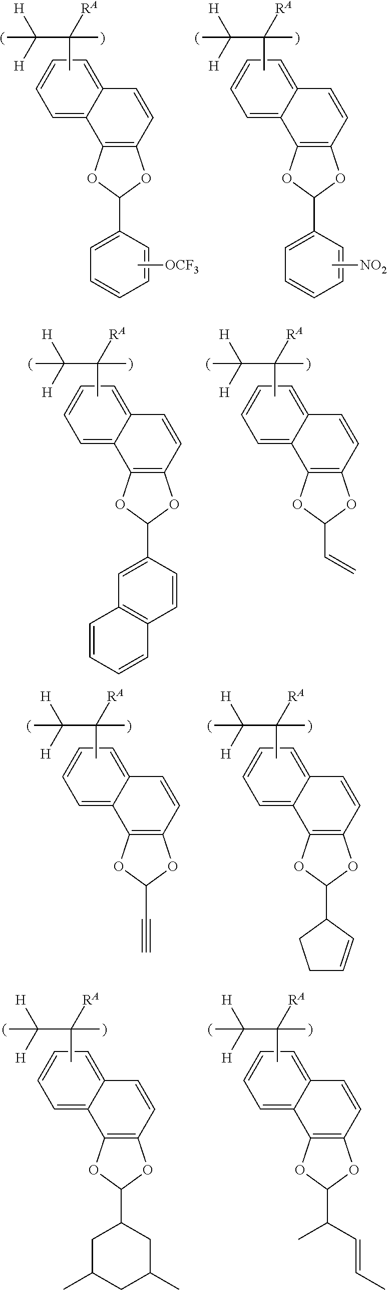

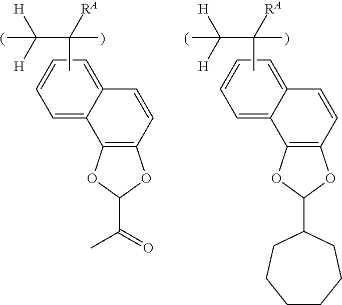

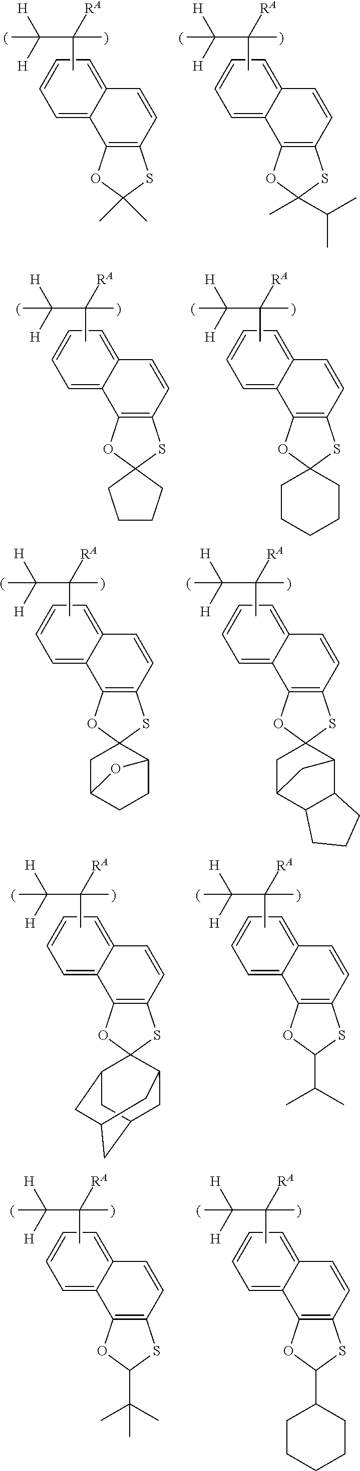

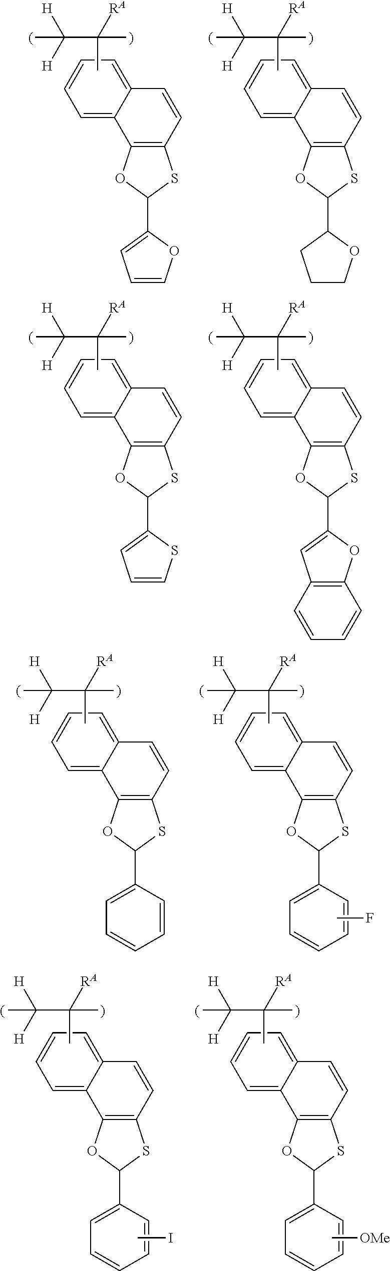

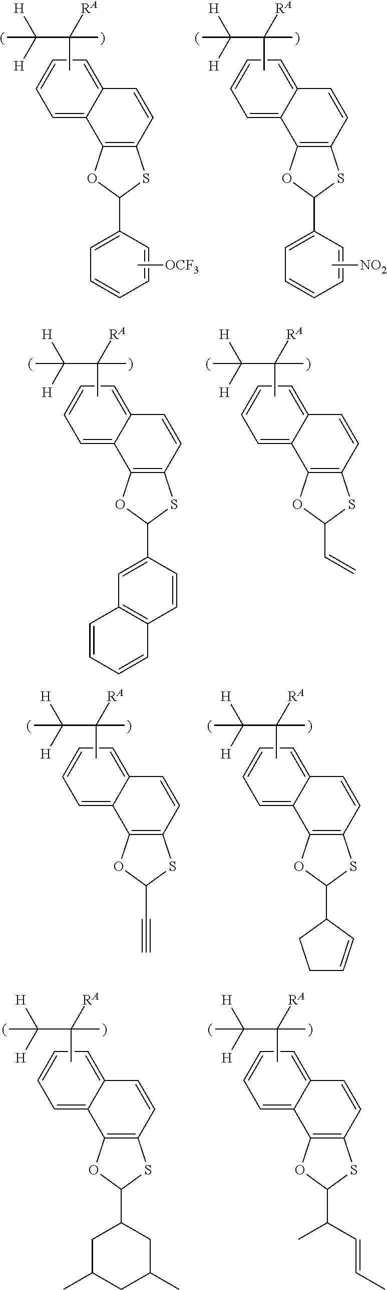

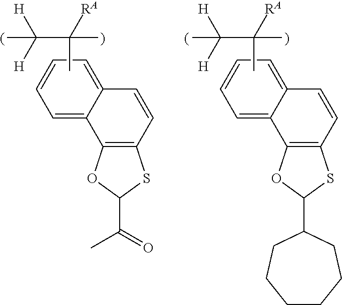

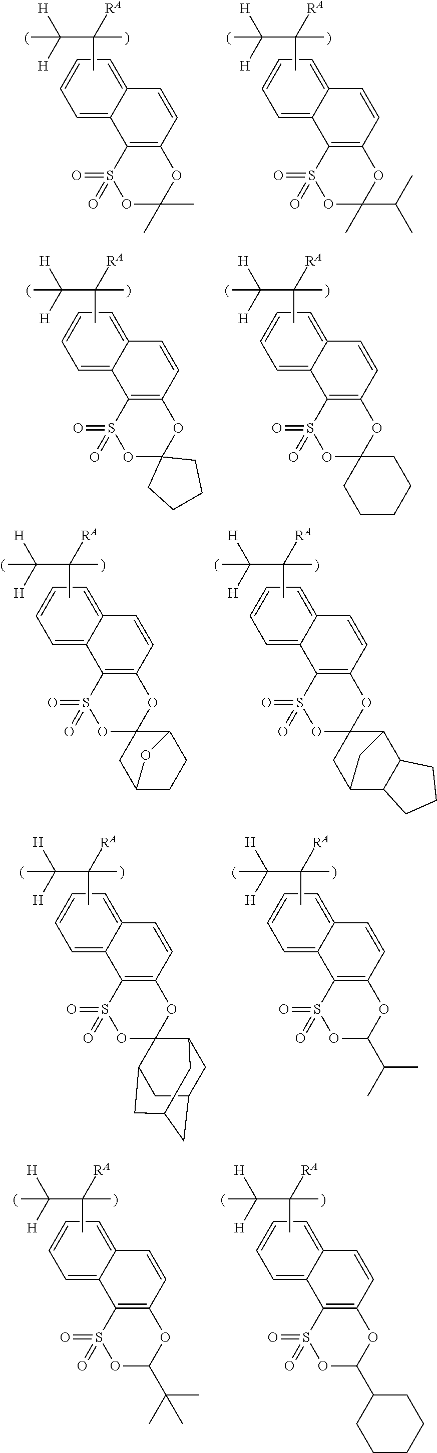

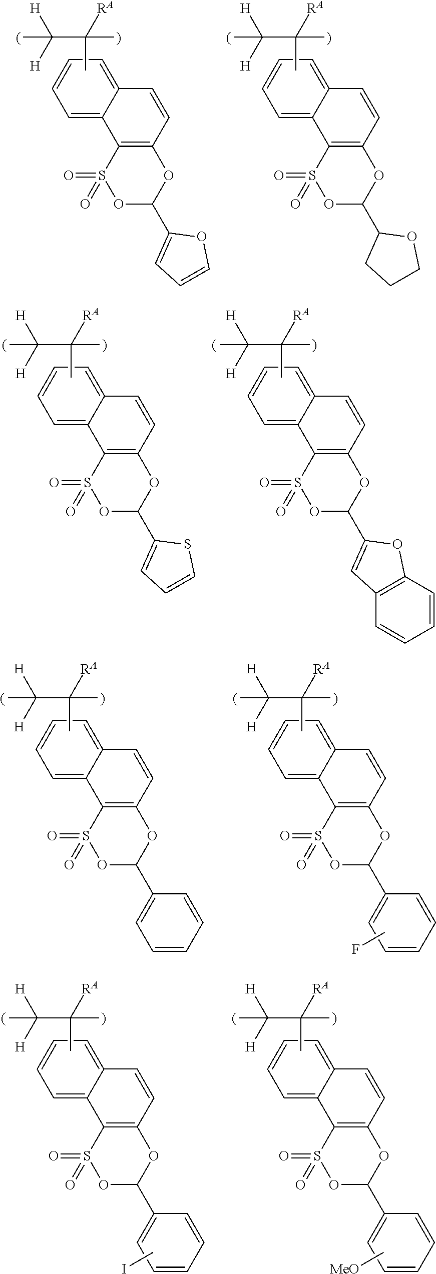

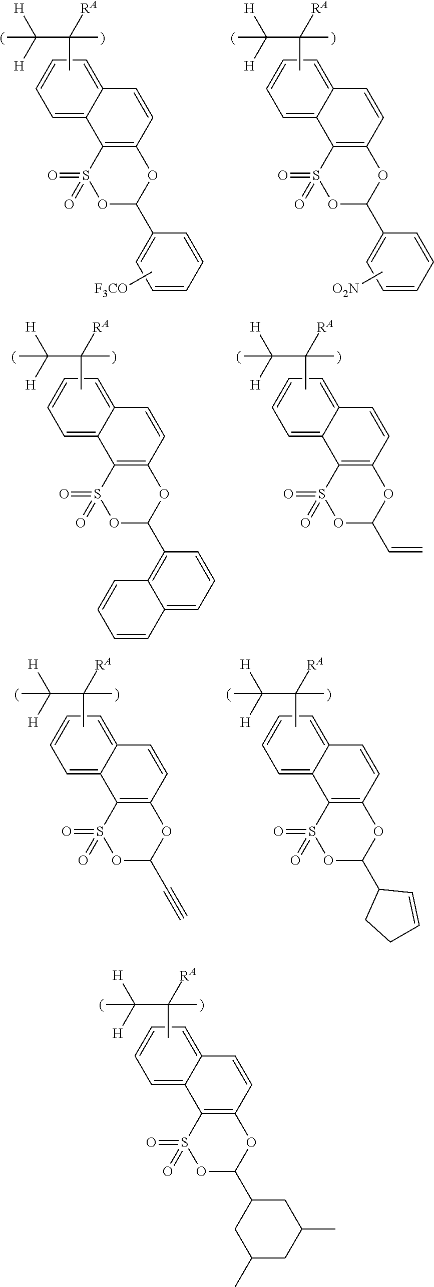

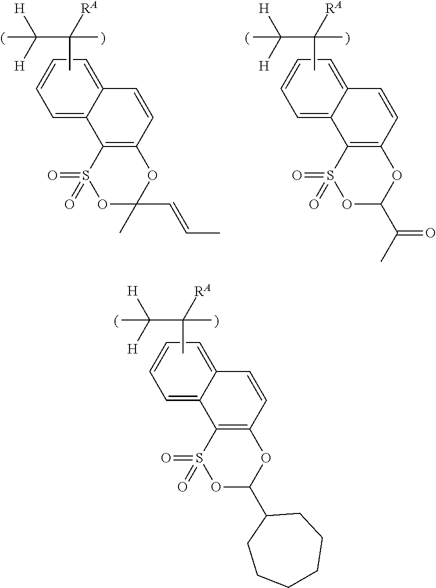

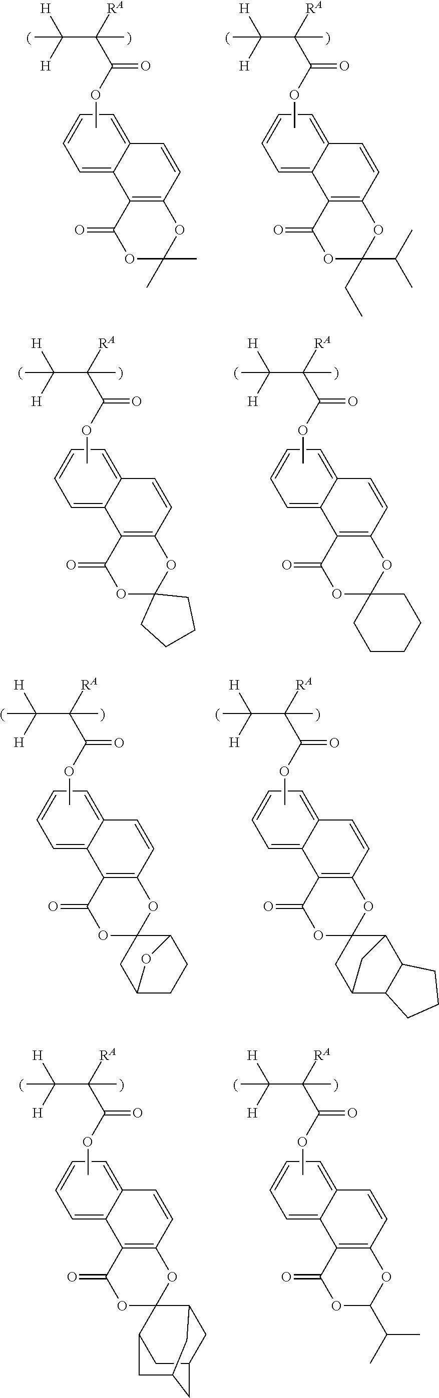

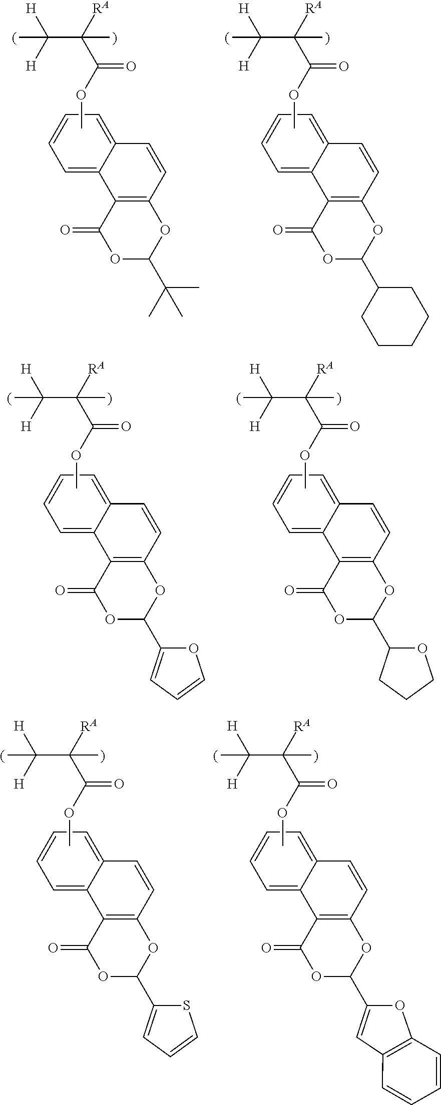

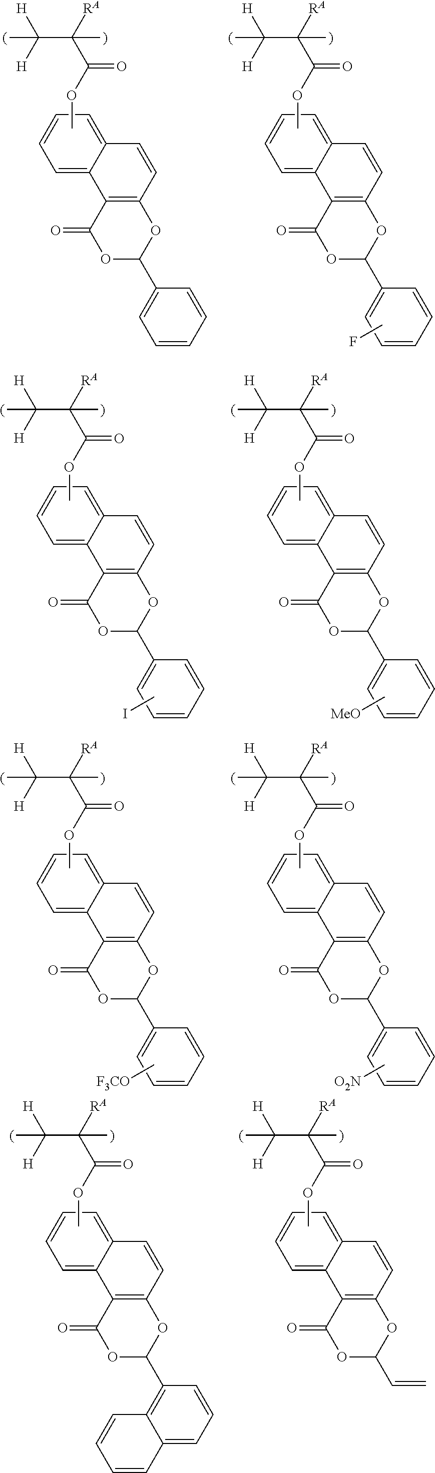

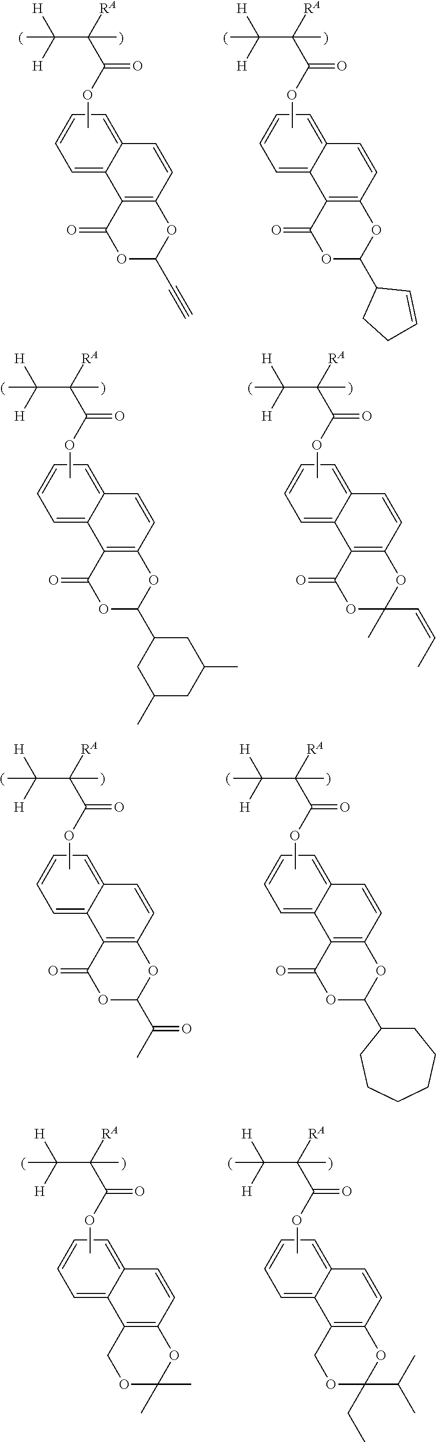

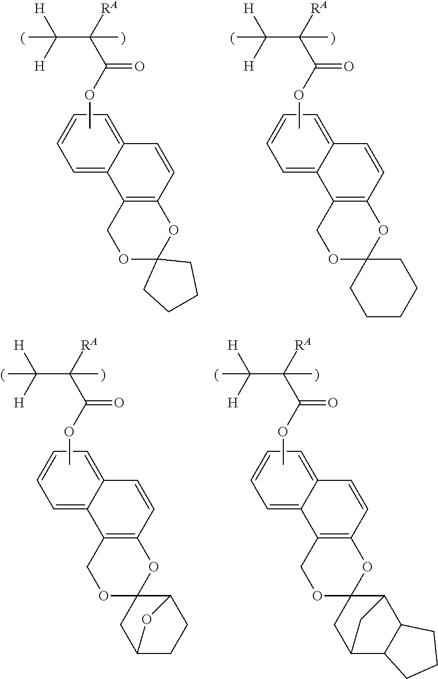

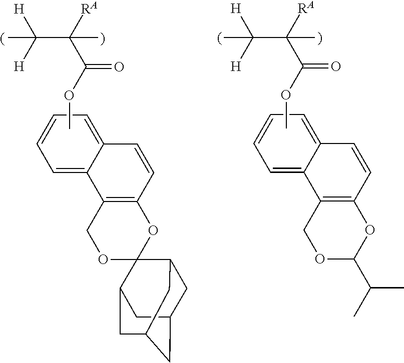

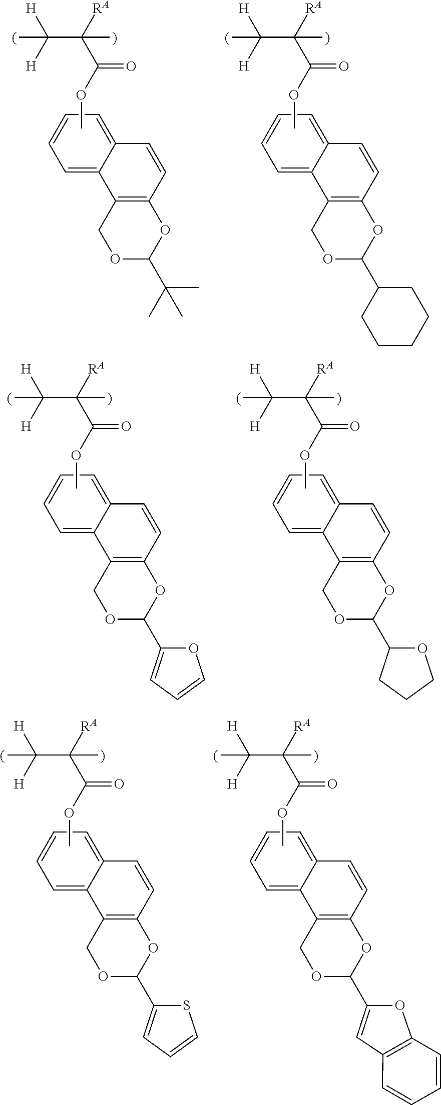

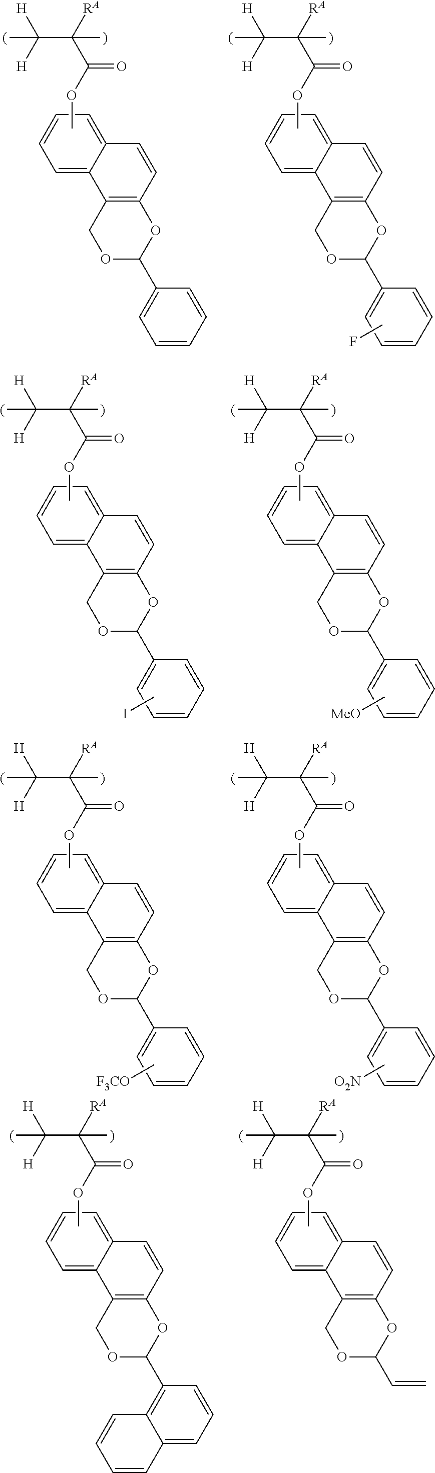

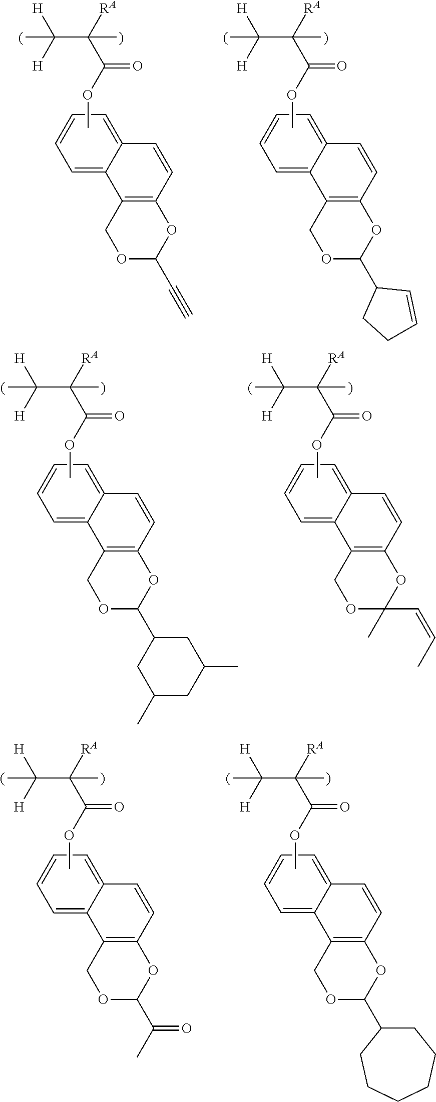

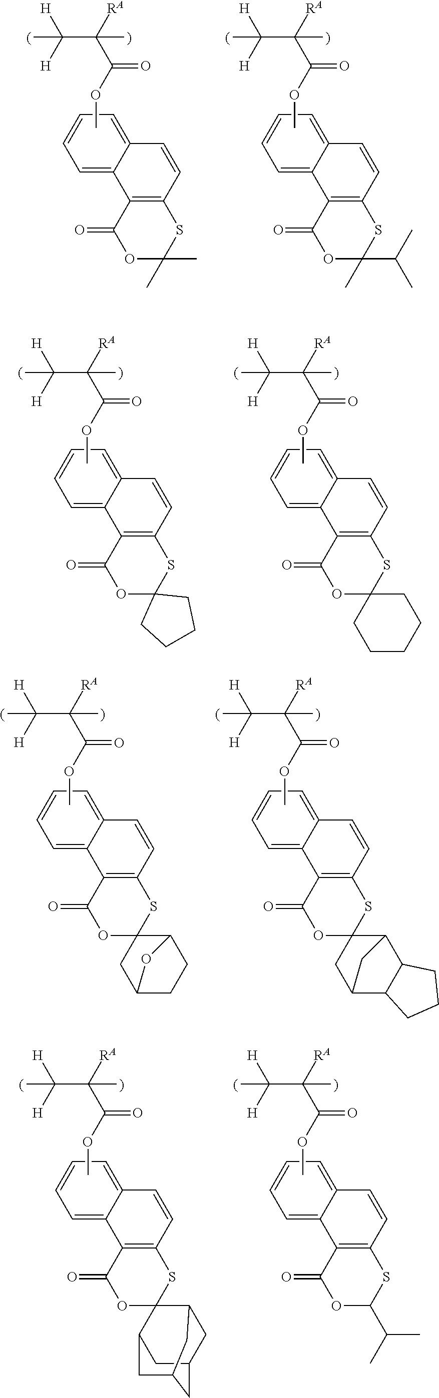

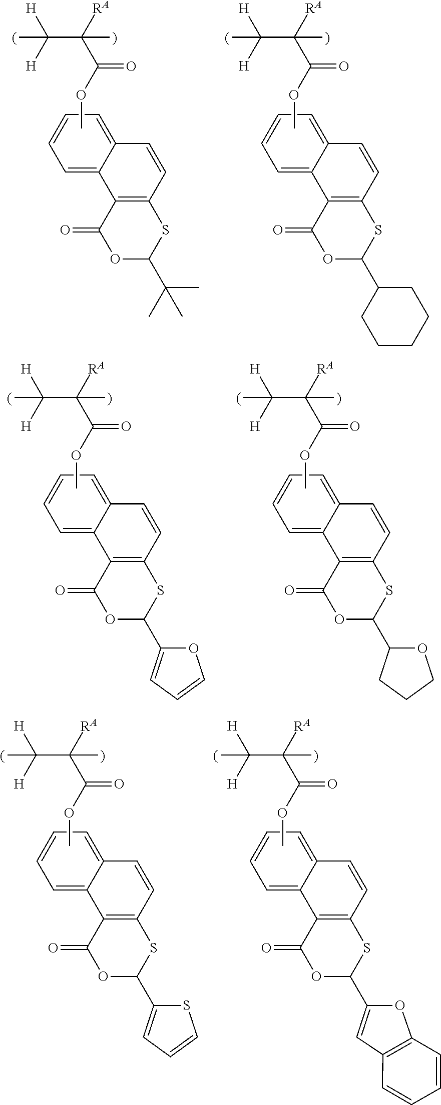

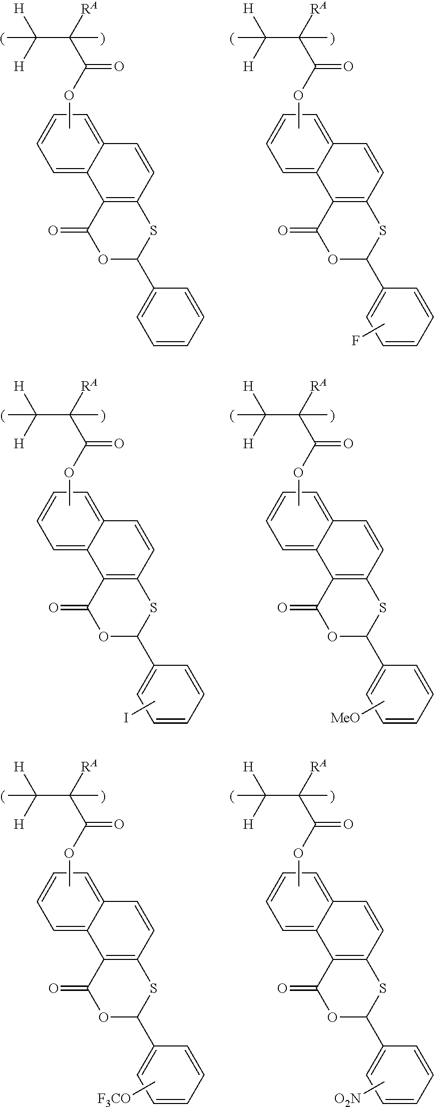

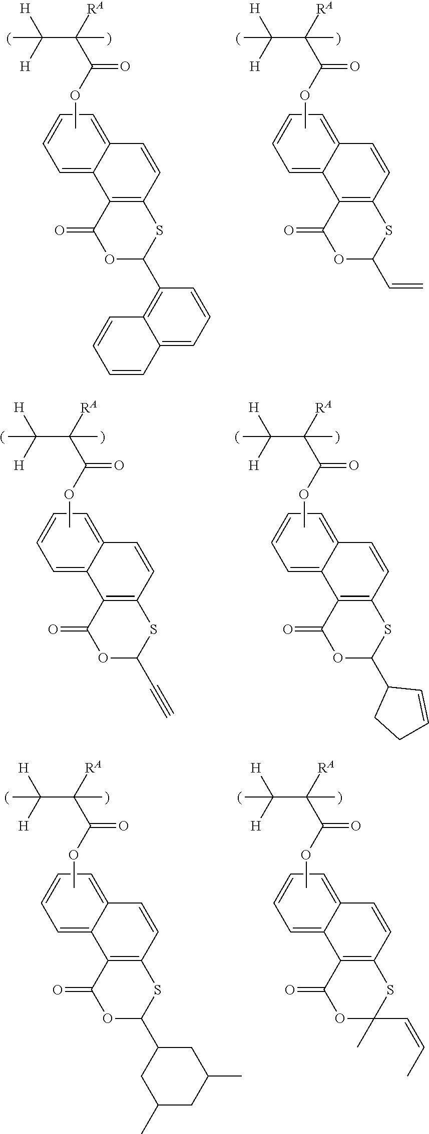

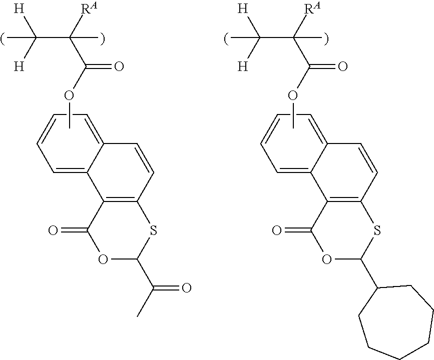

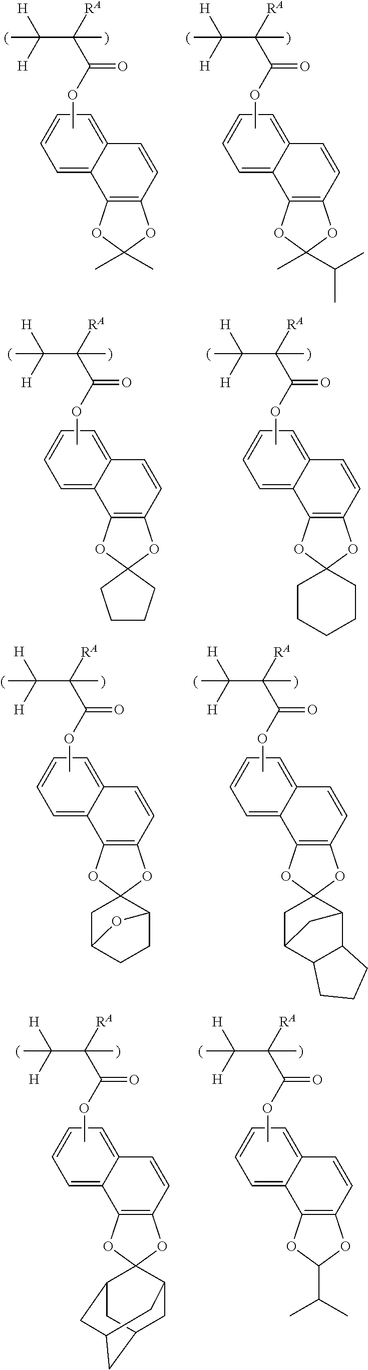

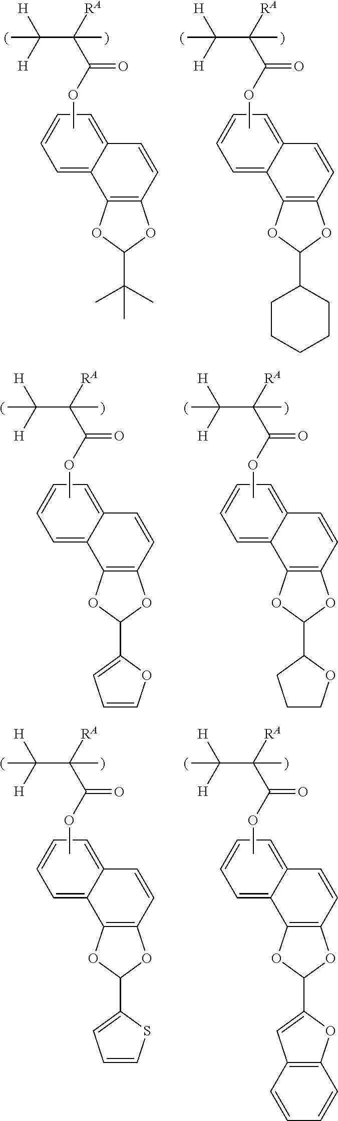

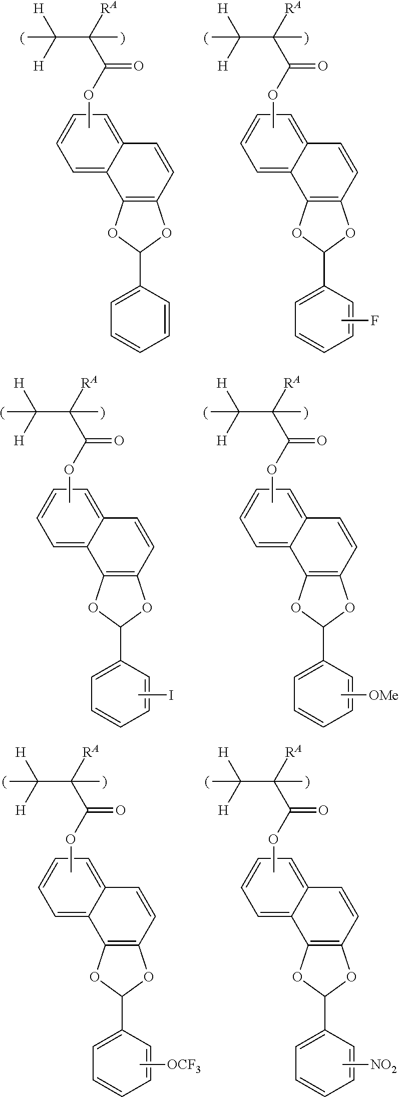

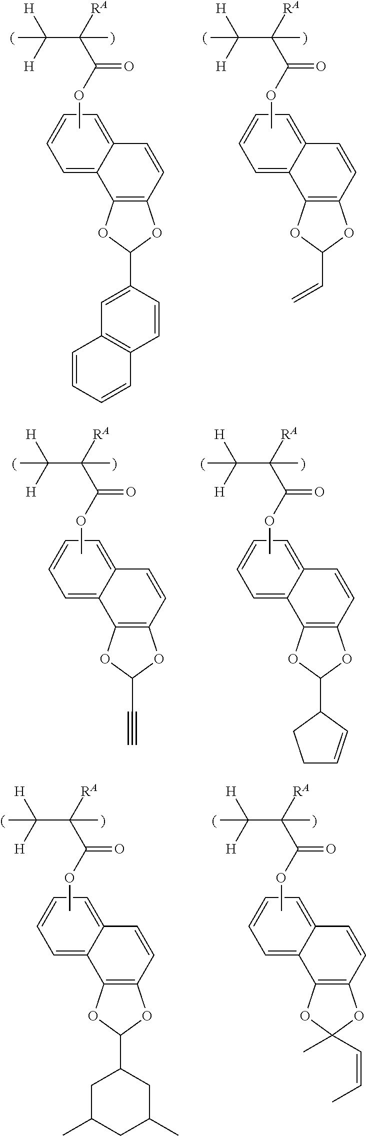

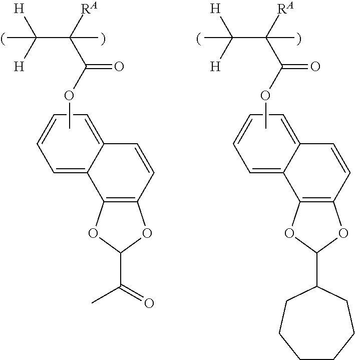

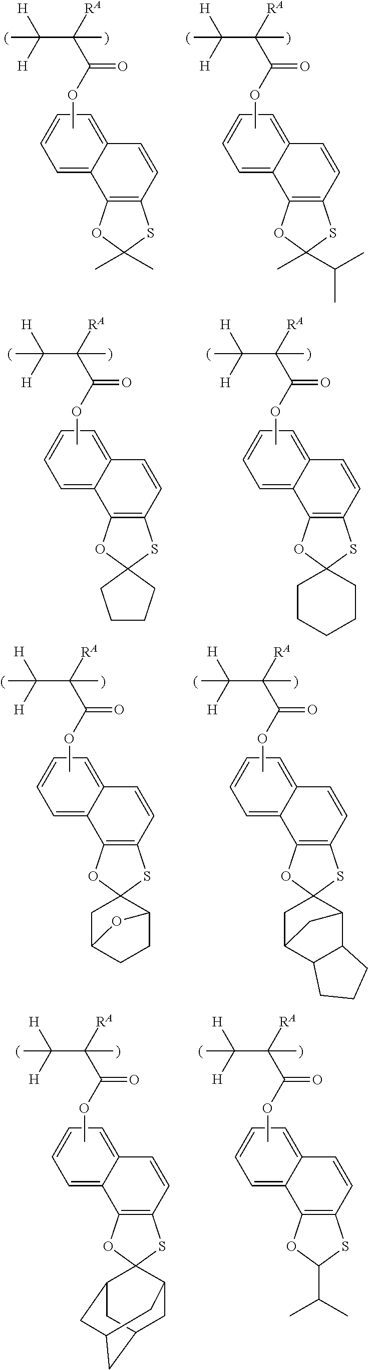

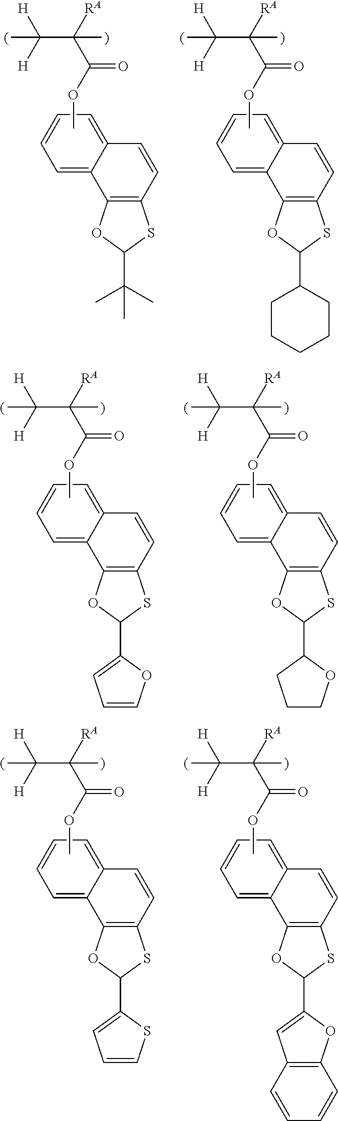

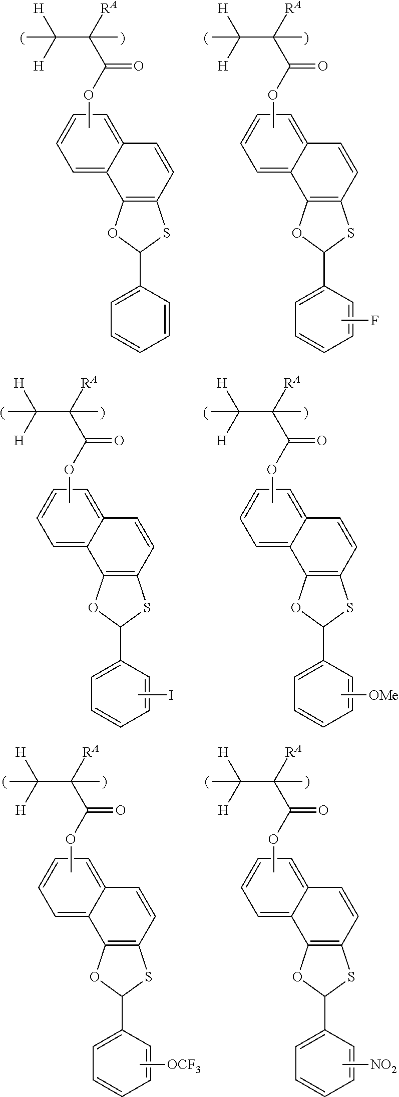

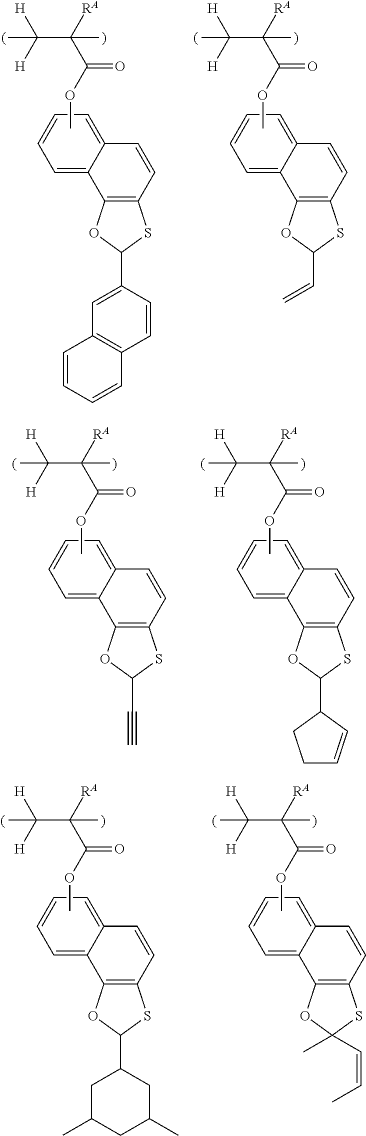

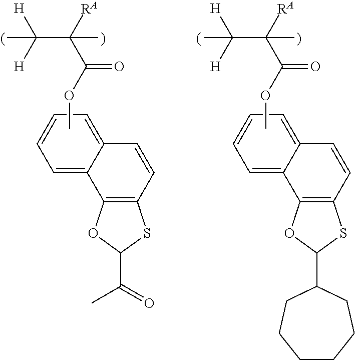

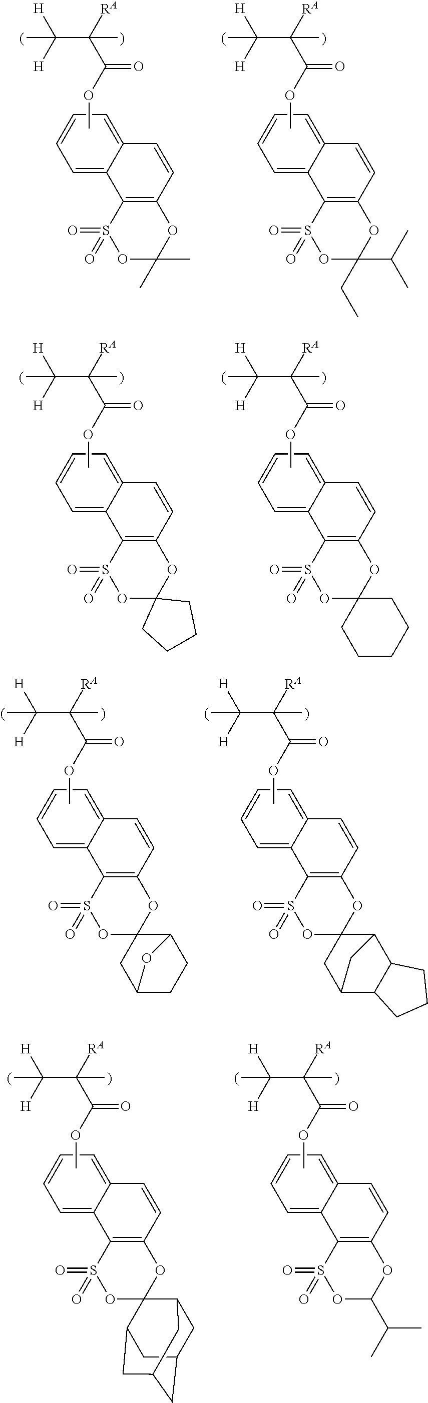

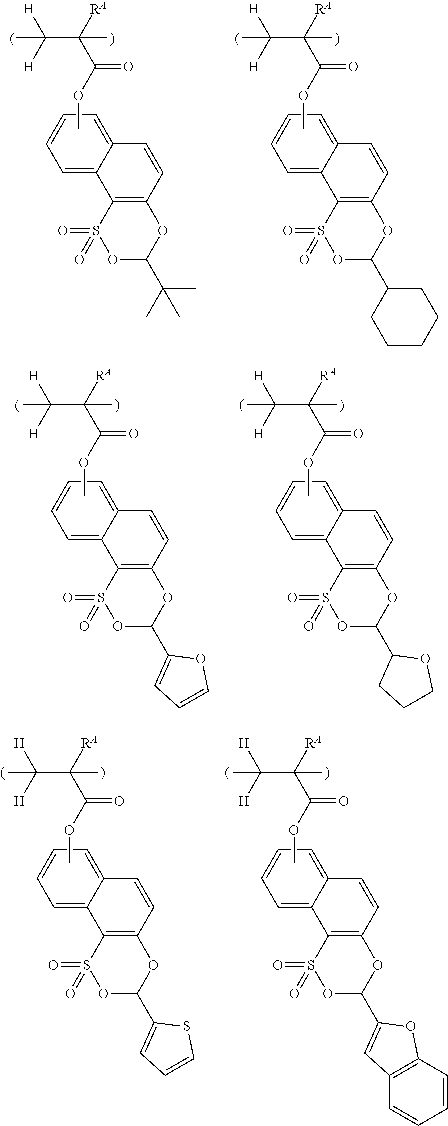

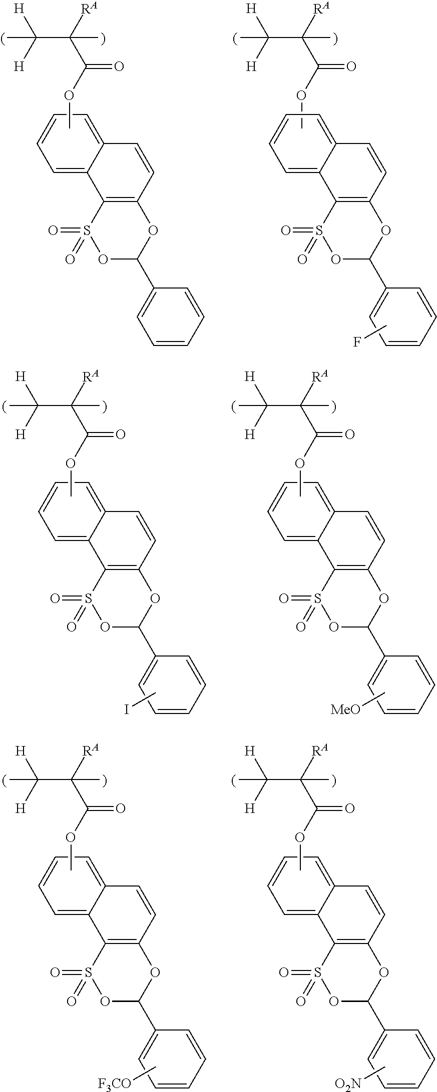

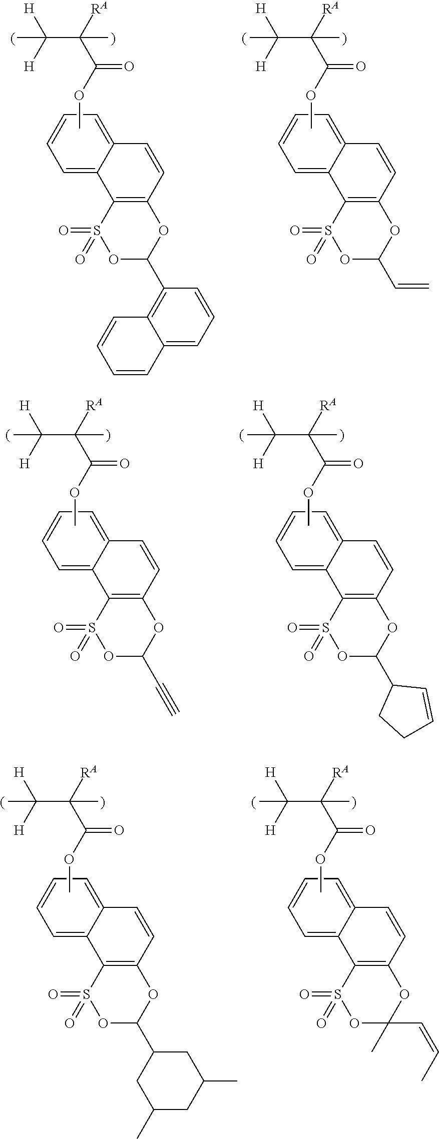

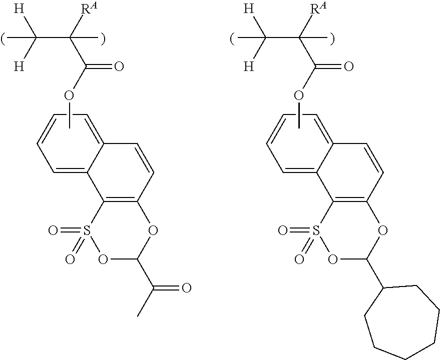

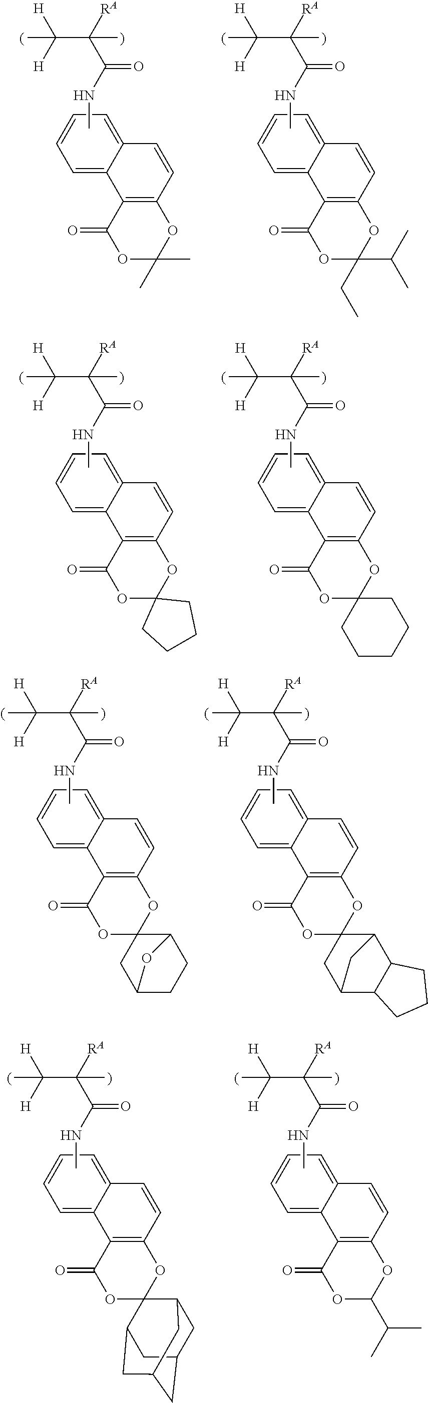

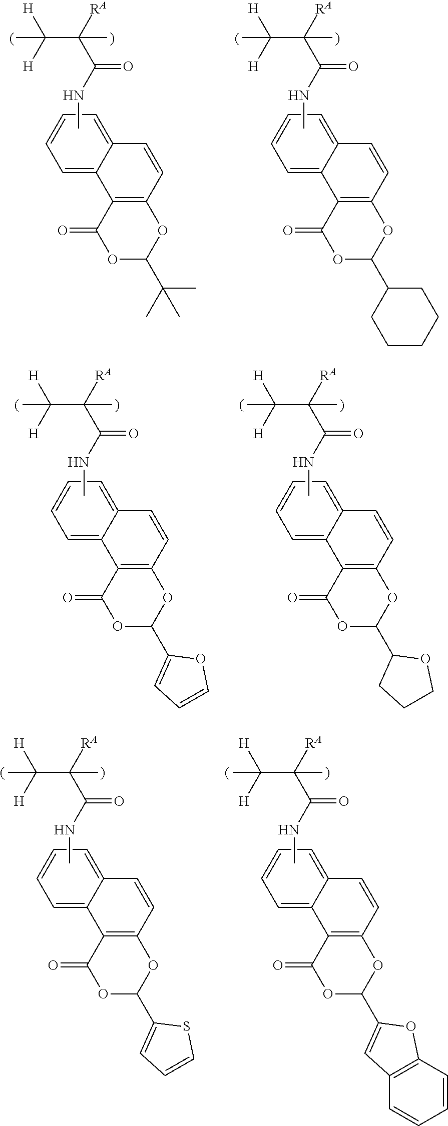

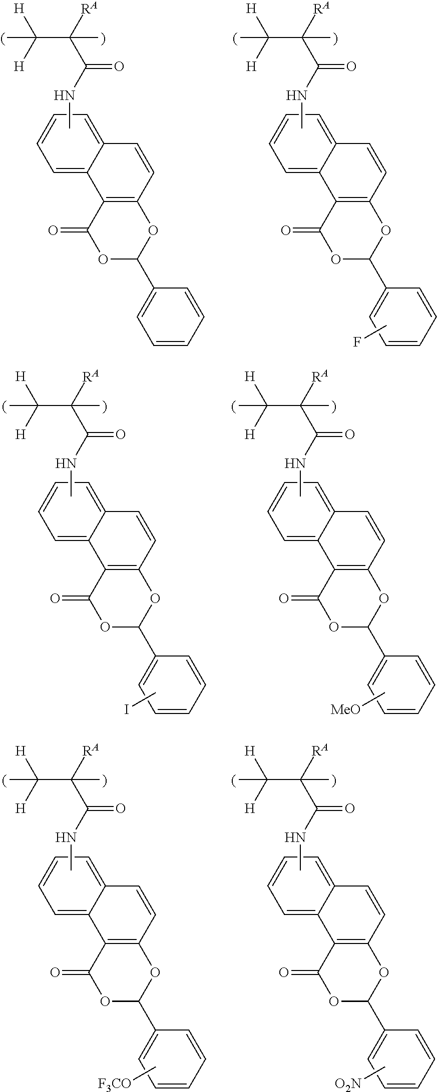

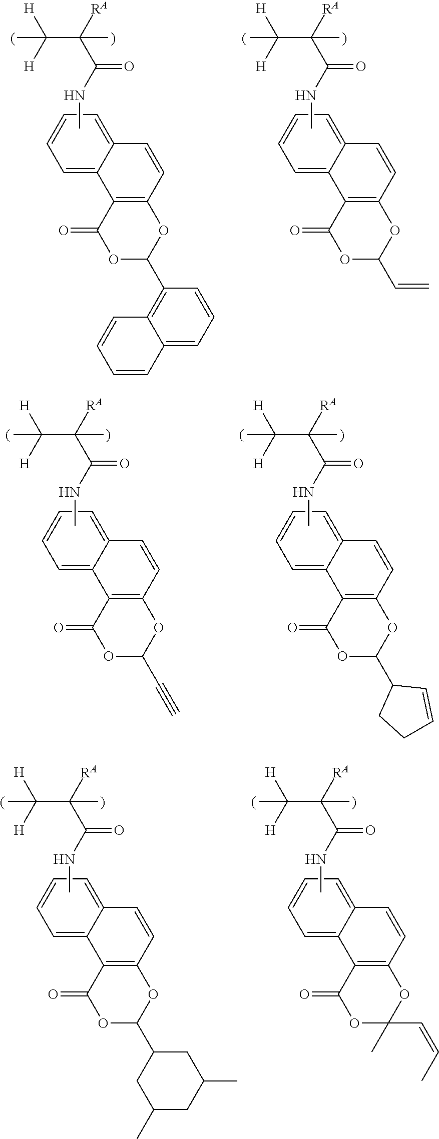

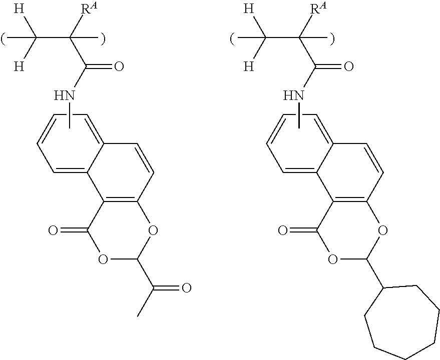

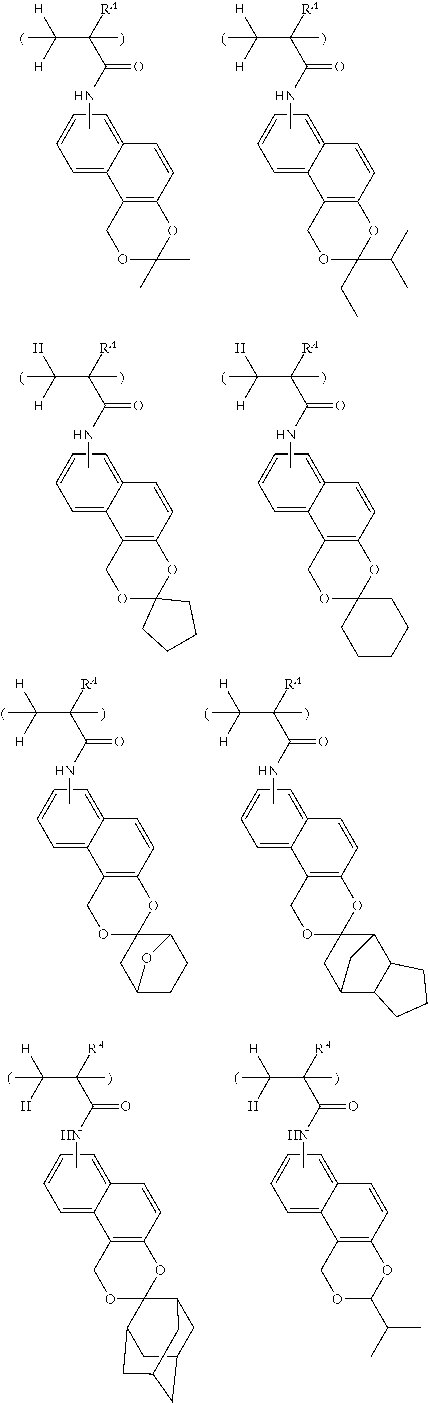

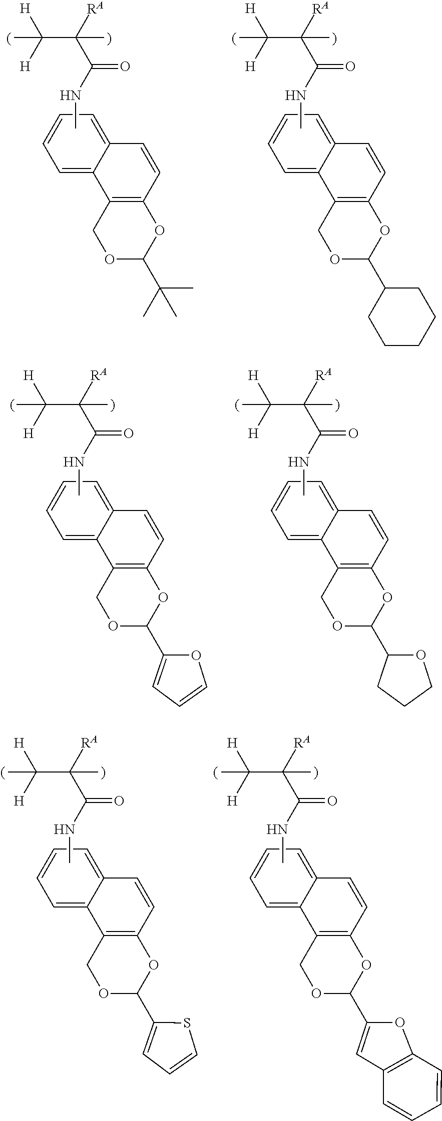

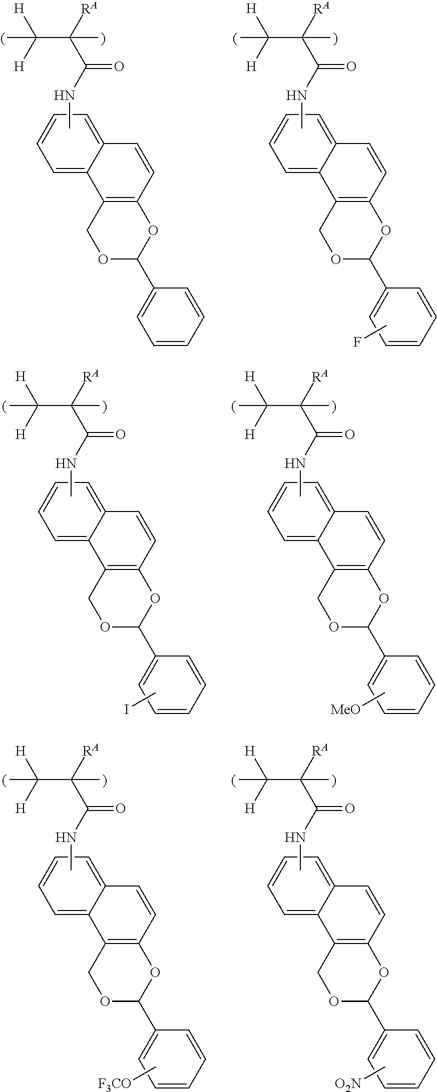

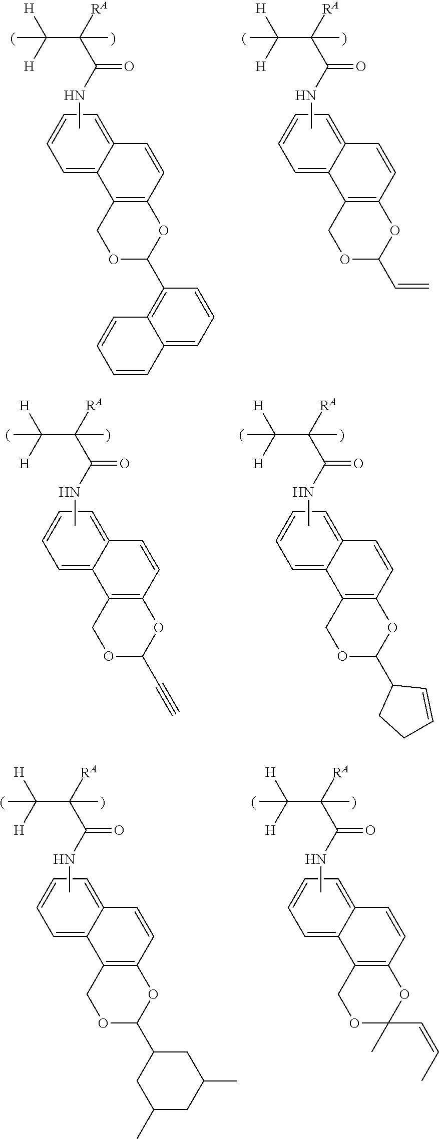

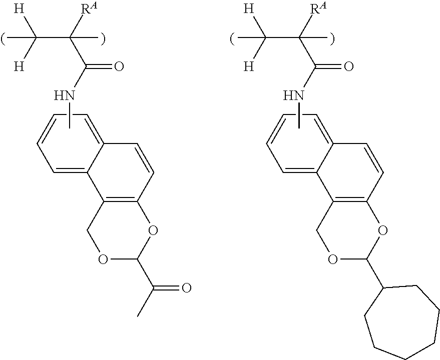

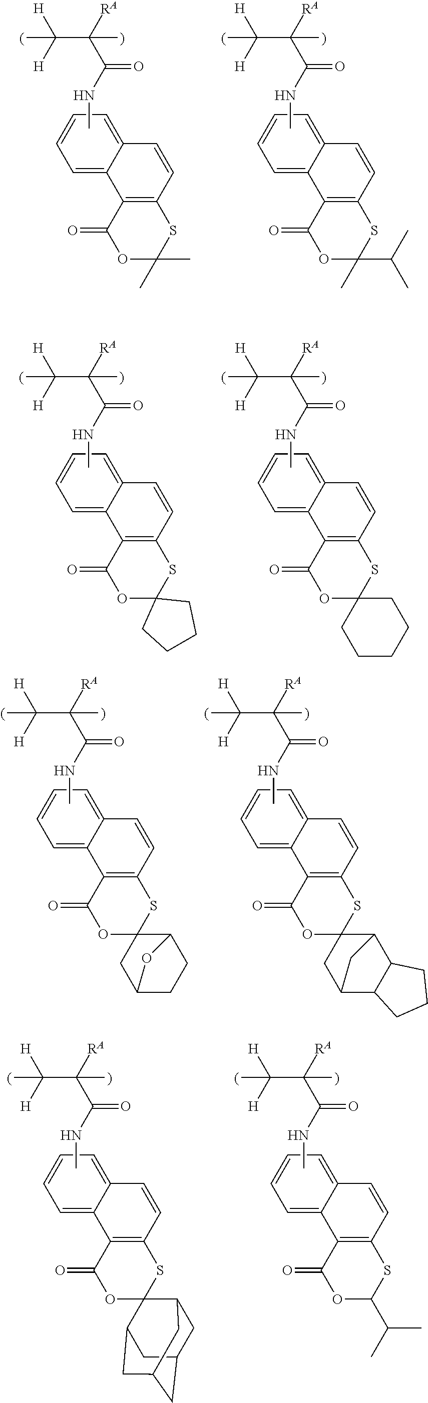

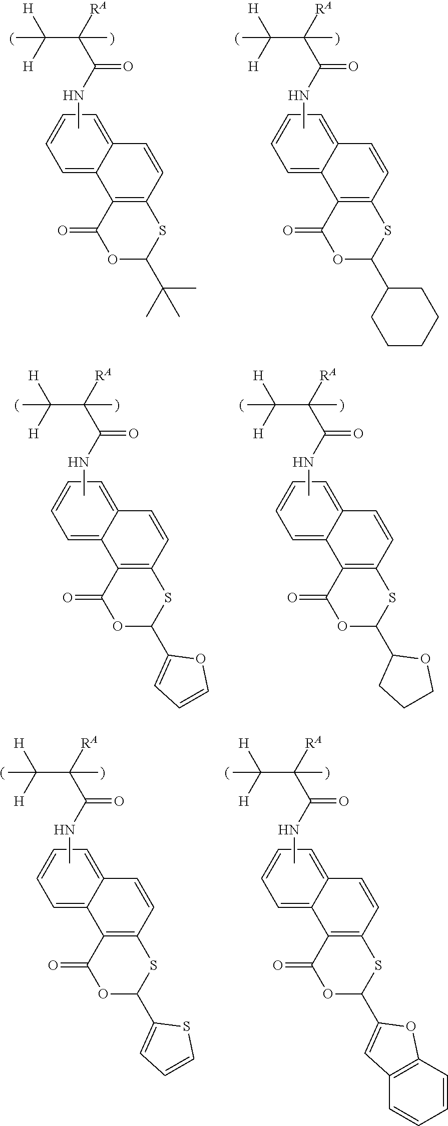

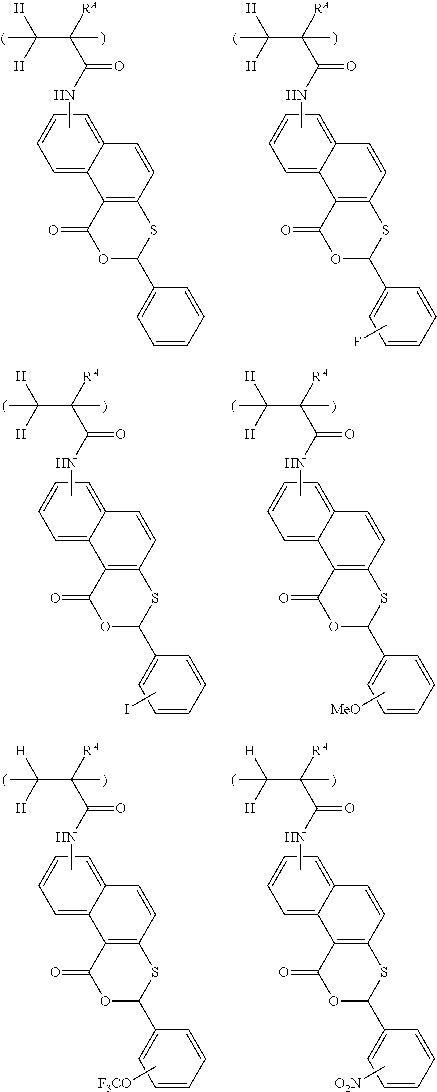

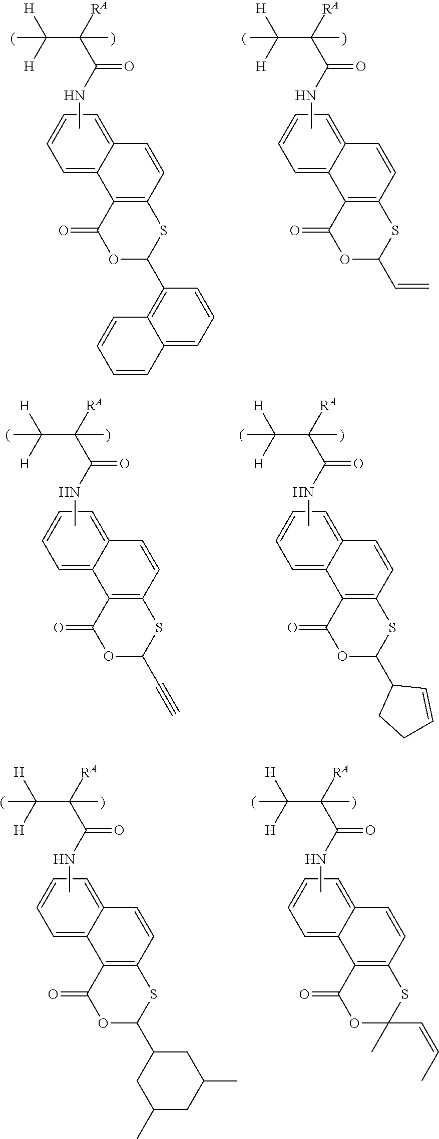

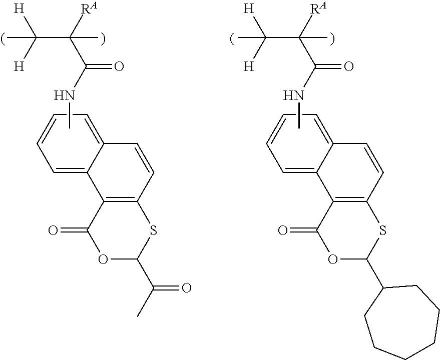

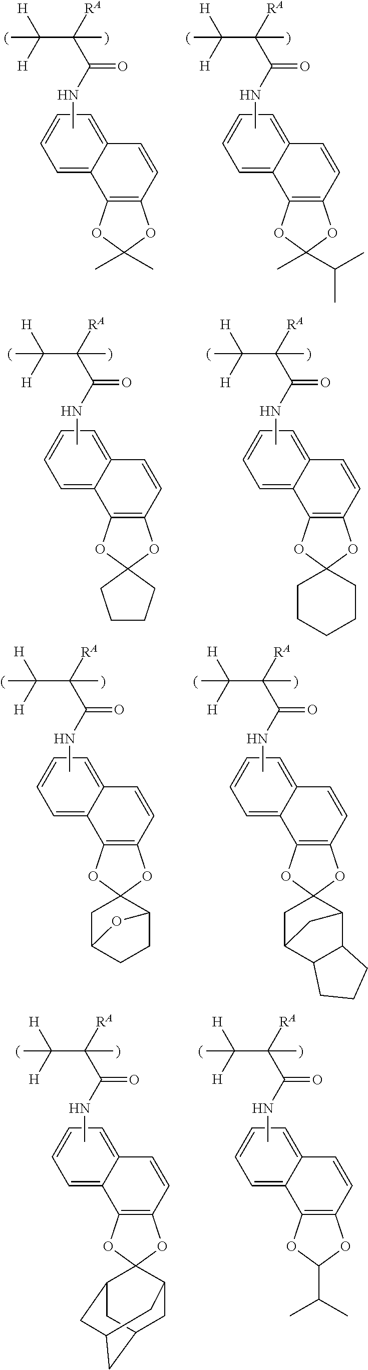

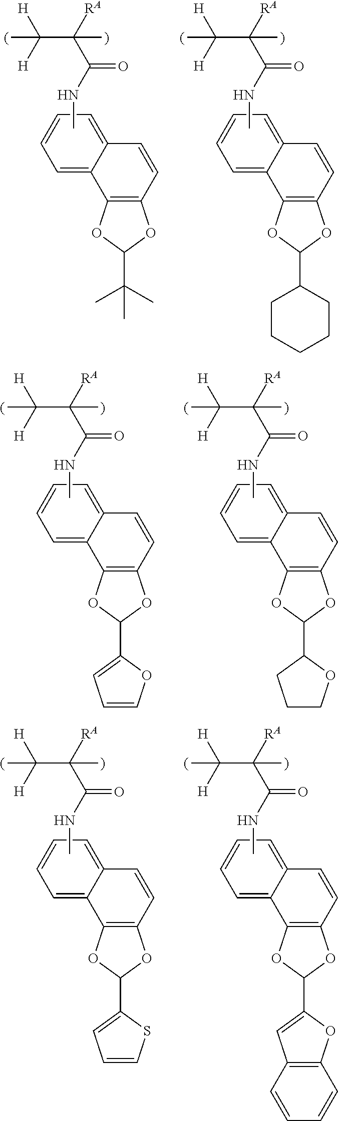

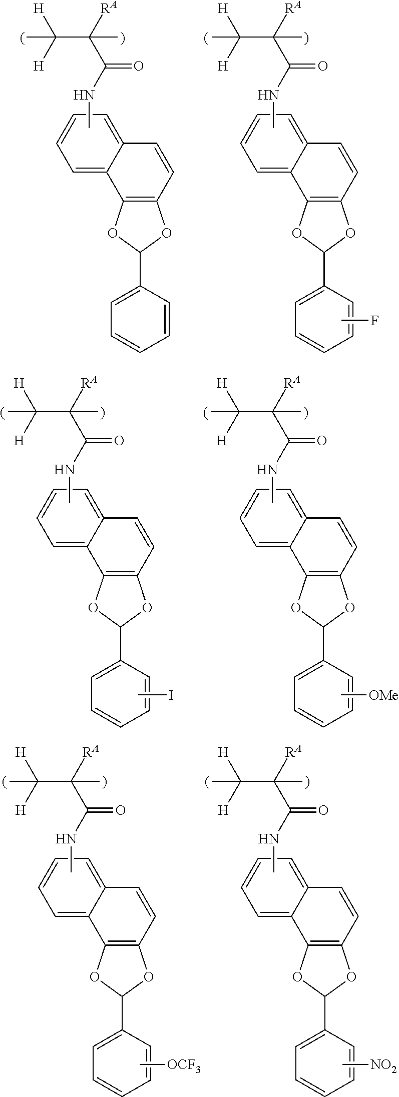

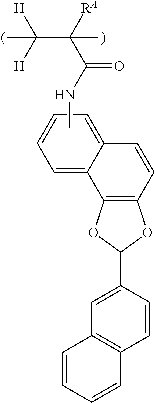

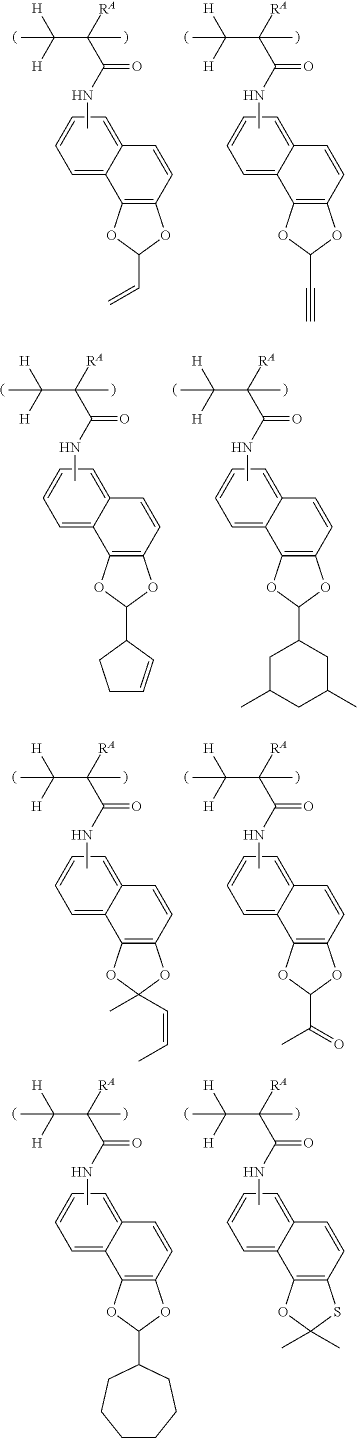

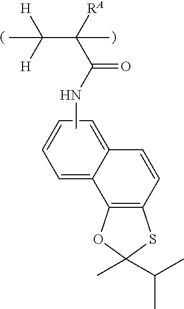

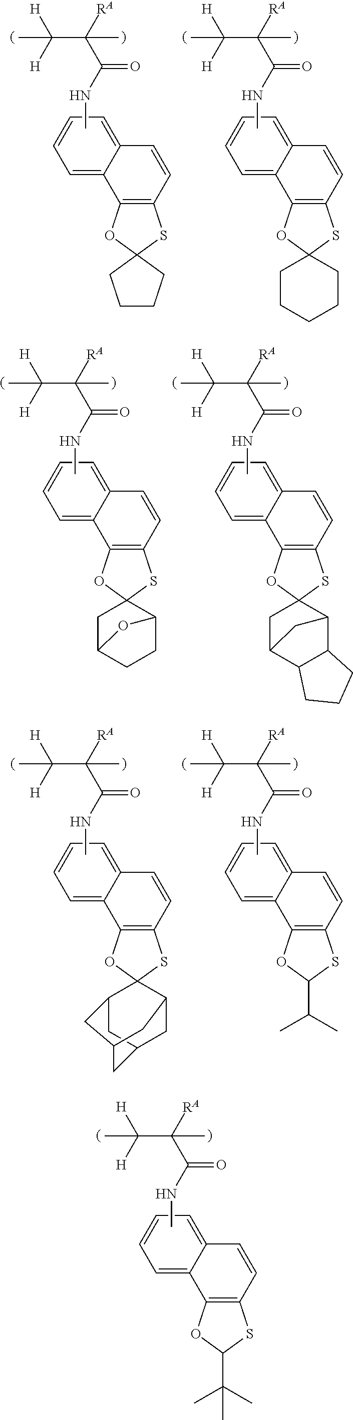

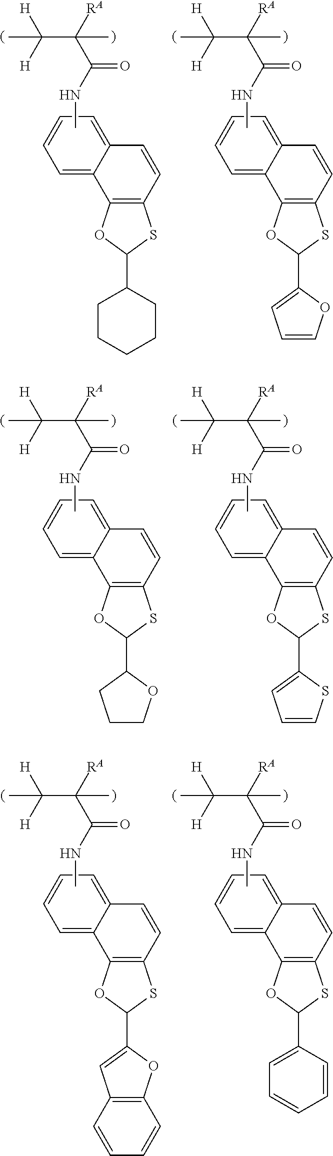

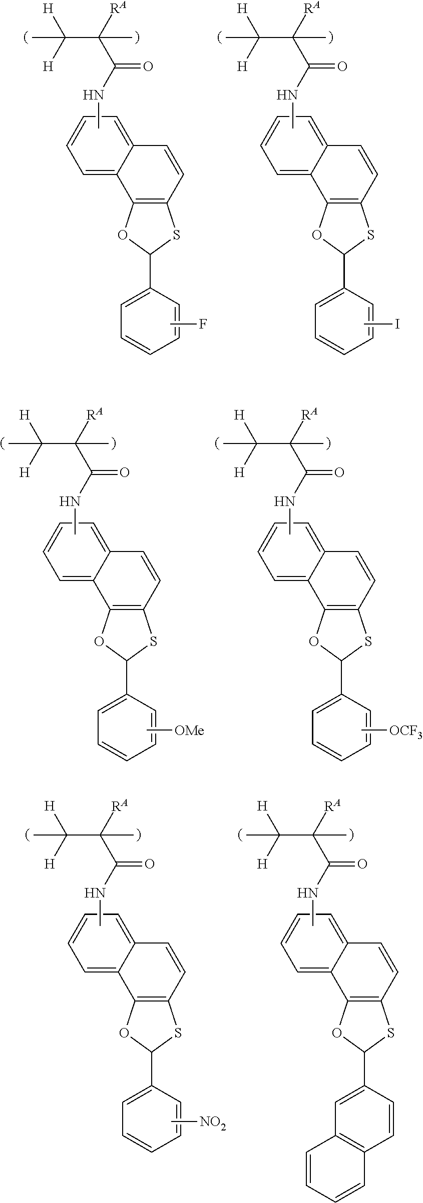

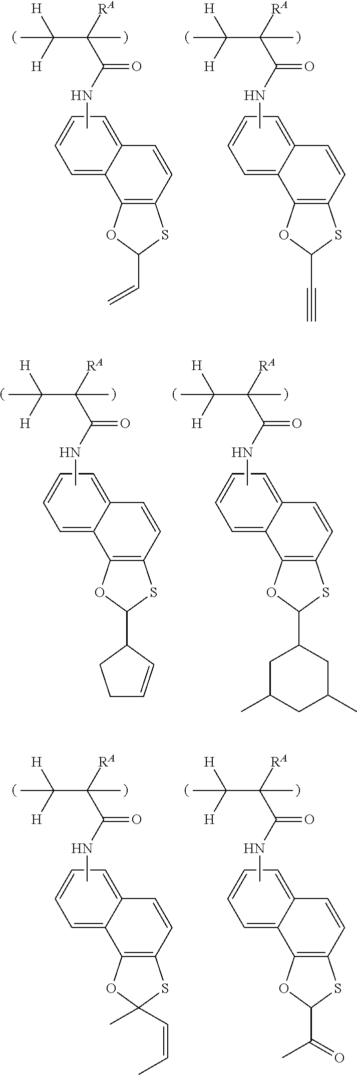

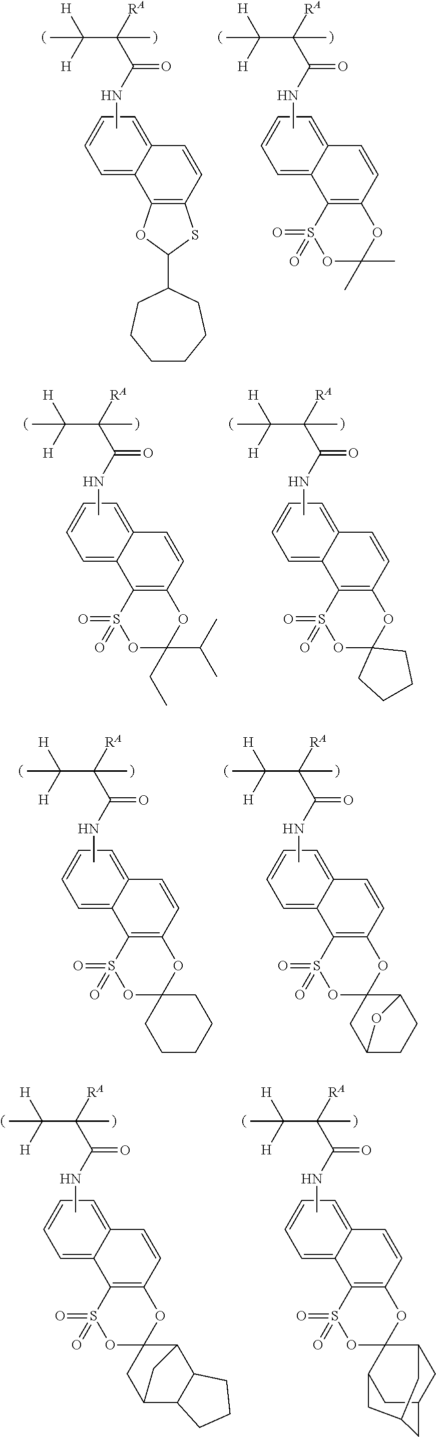

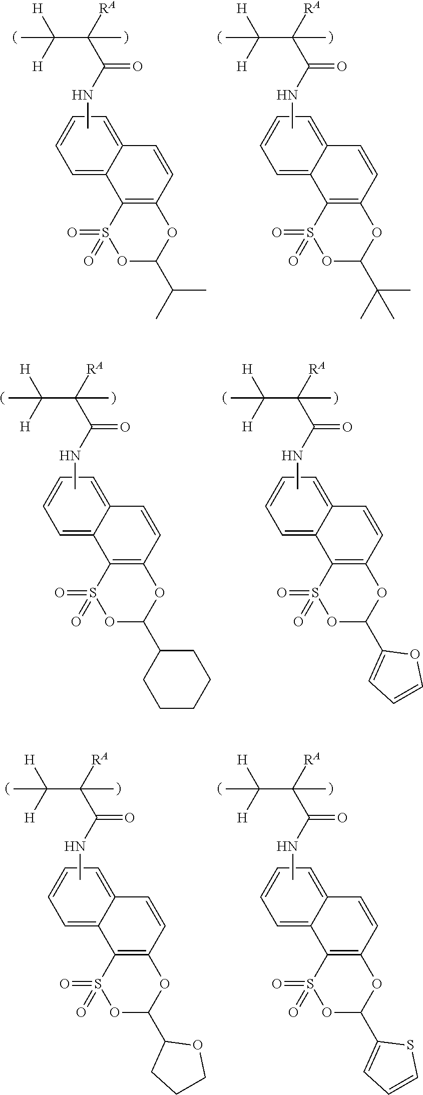

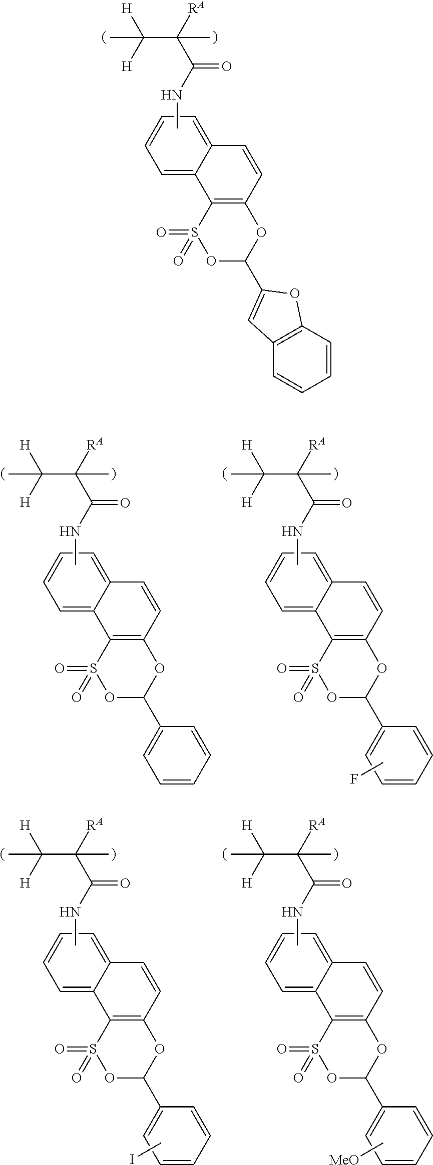

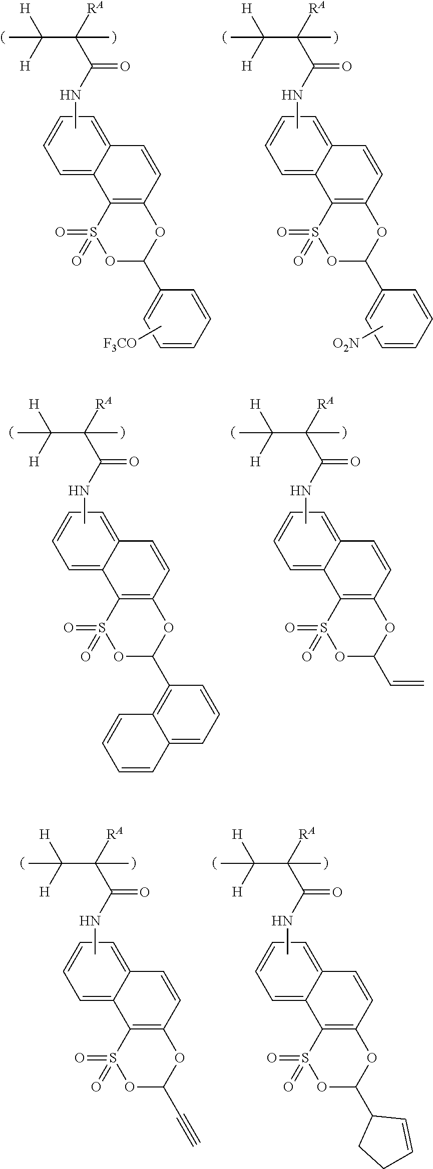

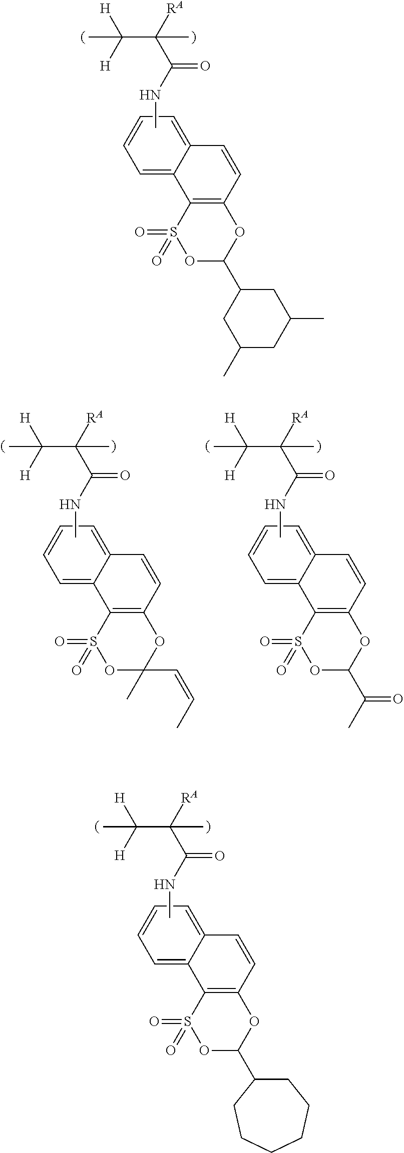

The polymer preferably further includes a repeating unit represented by the following formula (c) (hereinafter, also referred to as repeating unit-c).

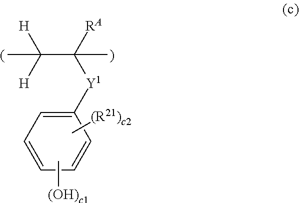

In the formula (c), R^{A} represents a hydrogen atom, a fluorine atom, a methyl group, or a trifluoromethyl group. Y¹ represents a single bond, *—C(═O)—O—, or *—C(═O)—NH—, and “*” represents an attachment point to the carbon atom of the main chain. R²¹ represents a halogen atom, a nitro group, a cyano group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom. “c1” represents 1, 2, 3, or 4 and “c2” represents 0, 1, 2, or 3, provided that 1≤c1+c2≤5.

Specific examples of the repeating unit-c include the following, but are not limited thereto. Note that, in the following formulae, R^{A} is as defined above.

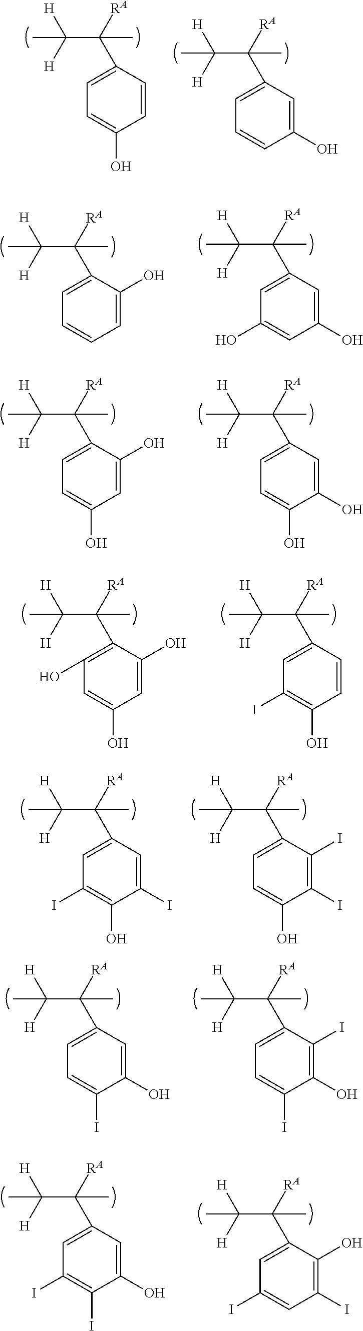

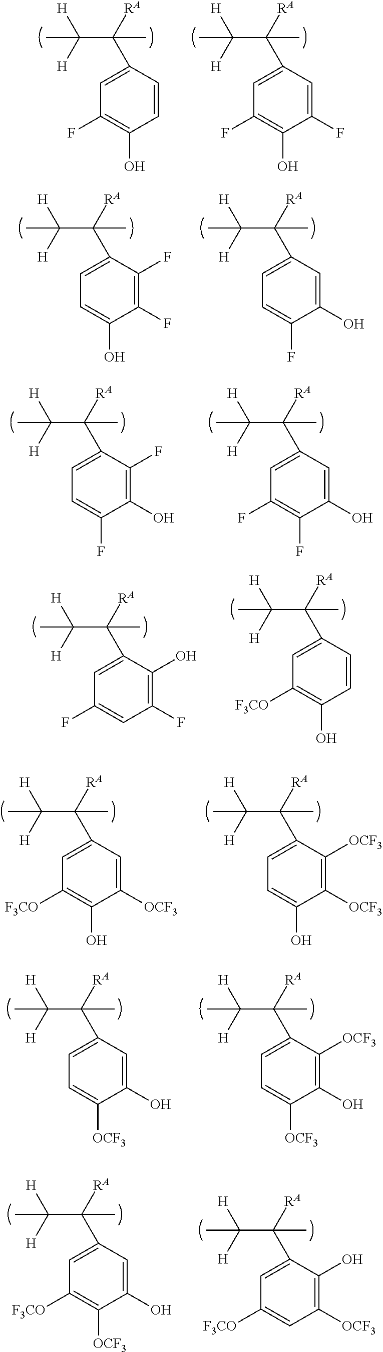

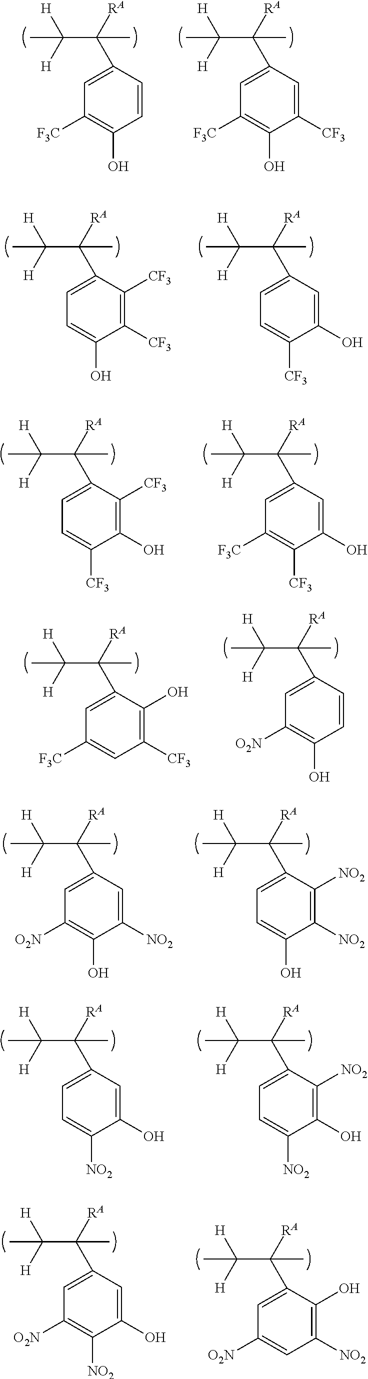

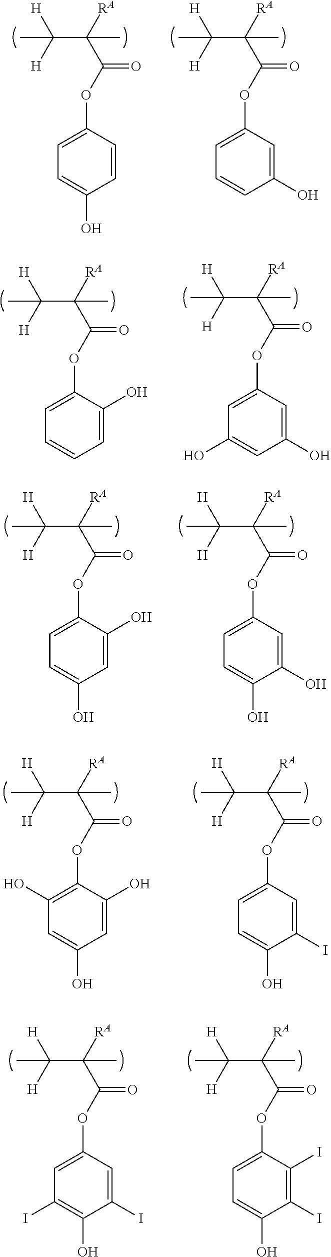

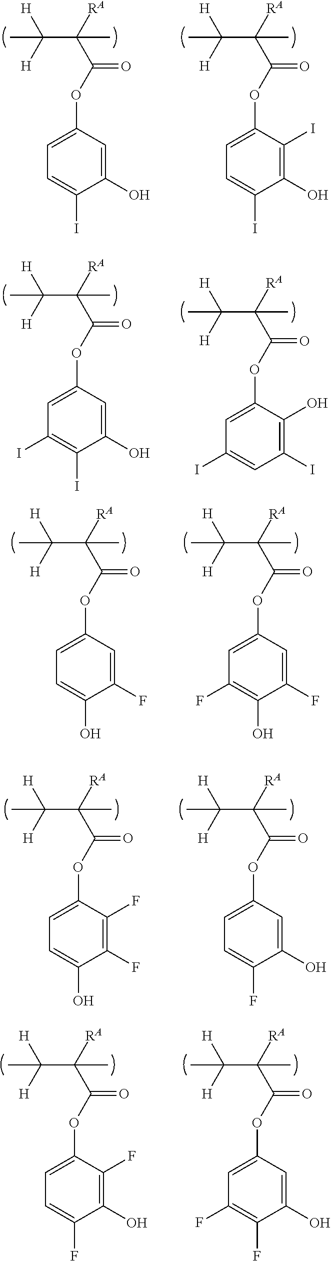

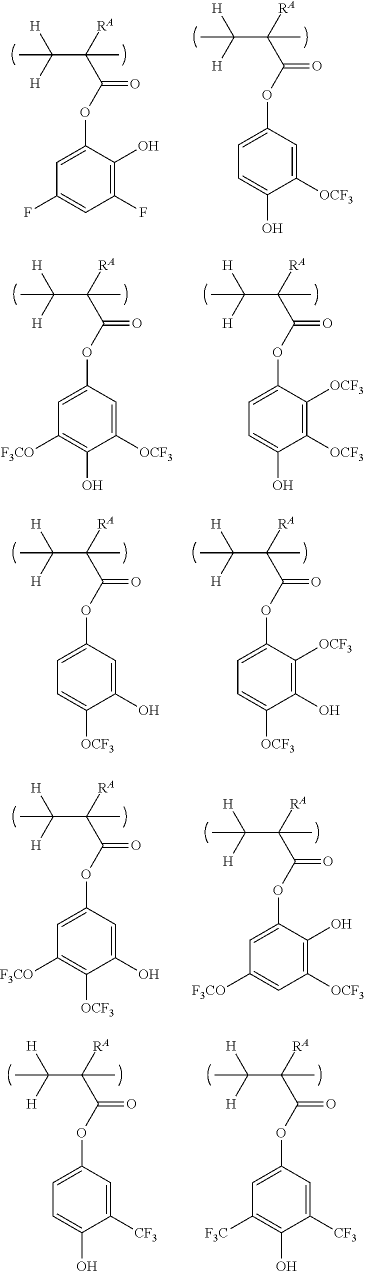

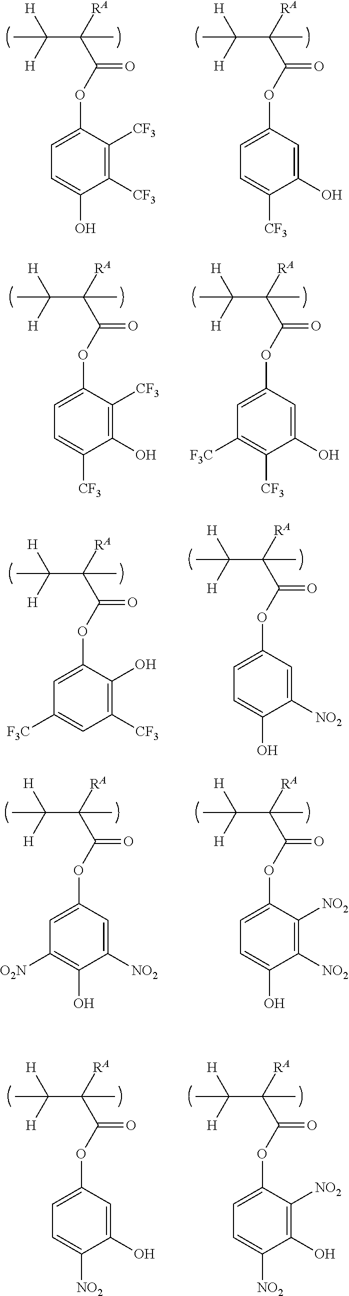

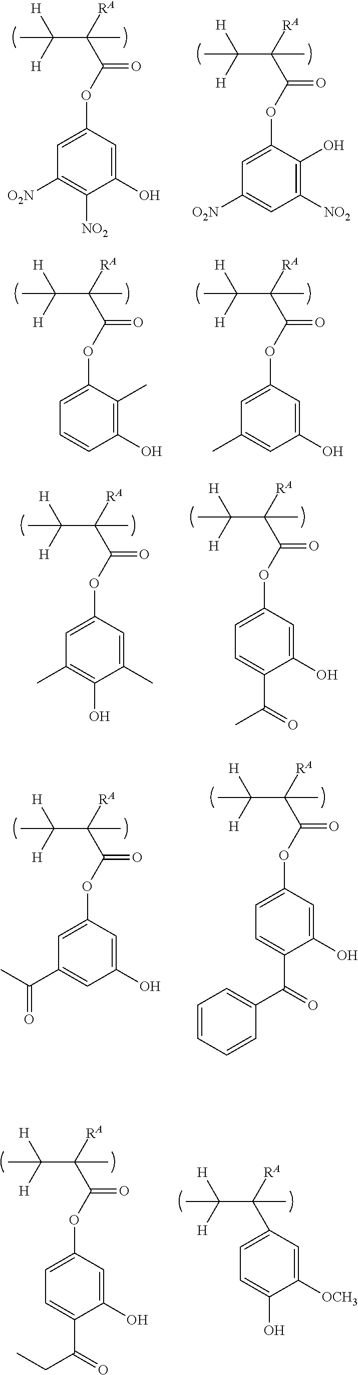

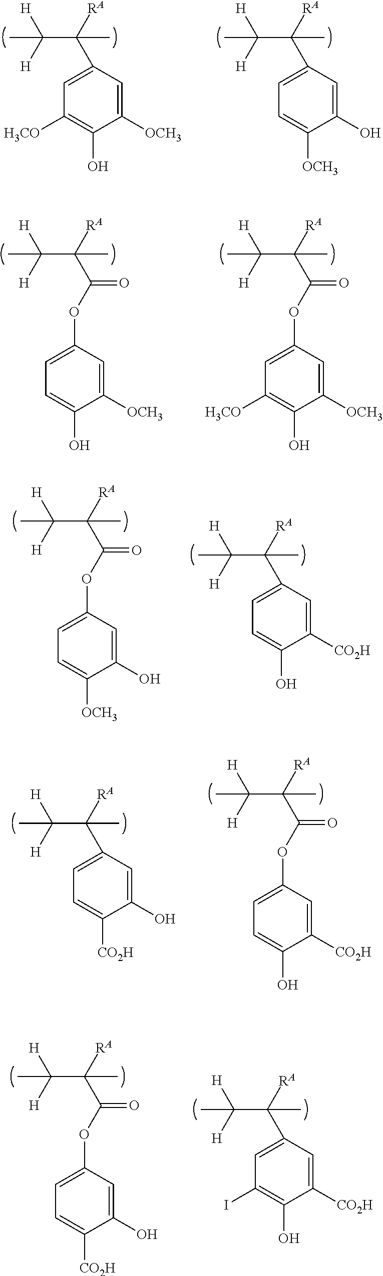

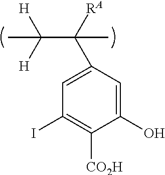

The polymer preferably further includes a repeating unit represented by the following formula (d) (hereinafter, also referred to as repeating unit-d).

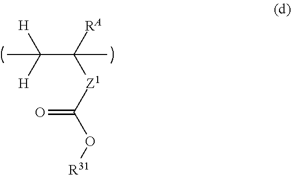

In the formula (d), R^{A} represents a hydrogen atom, a fluorine atom, a methyl group, or a trifluoromethyl group. Z¹ represents a single bond, a phenylene group, a naphthylene group, *—C(═O)—O—Z¹¹—, or *—C(═O)—NH—Z¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, “*” represents an attachment point to the carbon atom of the main chain, and Z¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring. R³¹ represents a hydrogen atom or a group having 1 to 20 carbon atoms and containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—).

Specific examples of the repeating unit-d include the following, but are not limited thereto. Note that, in the following formulae, R^{A} is as defined above.

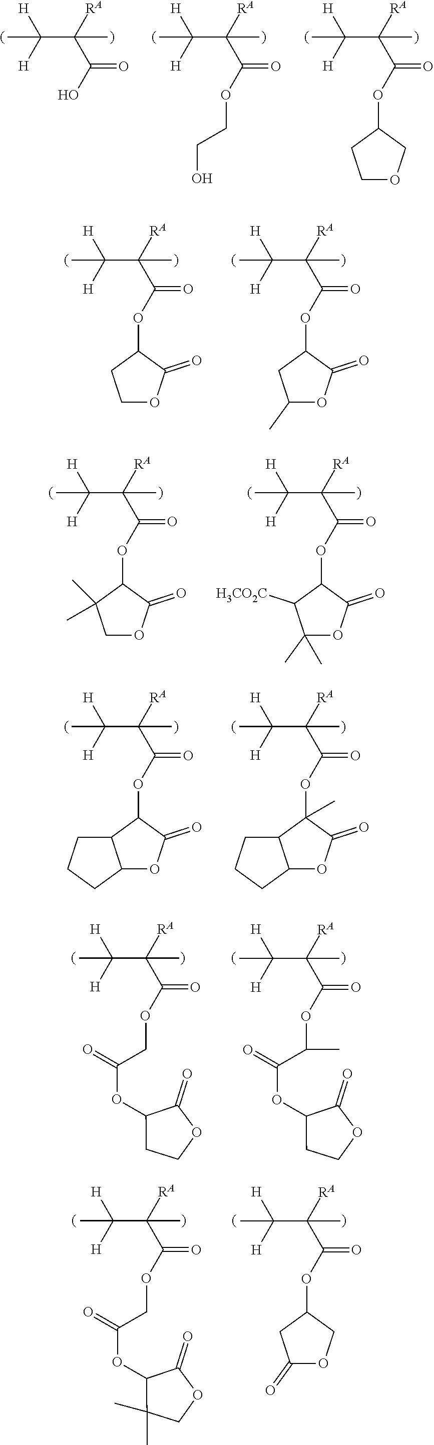

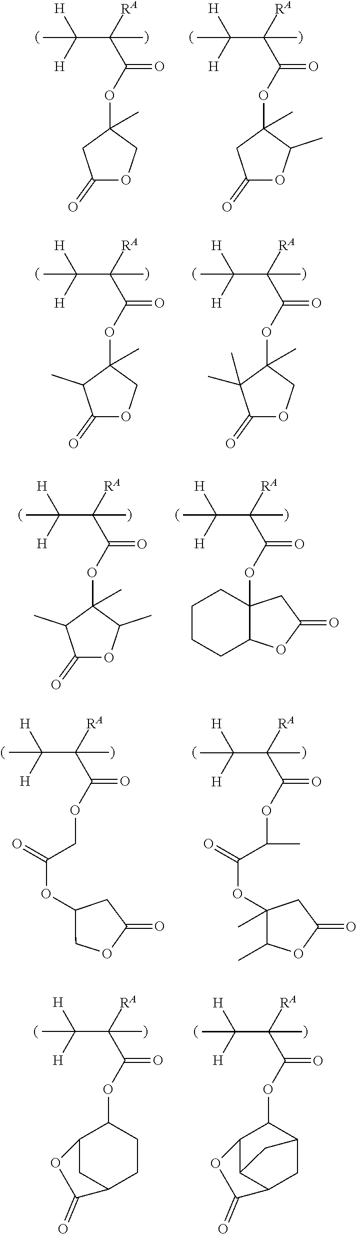

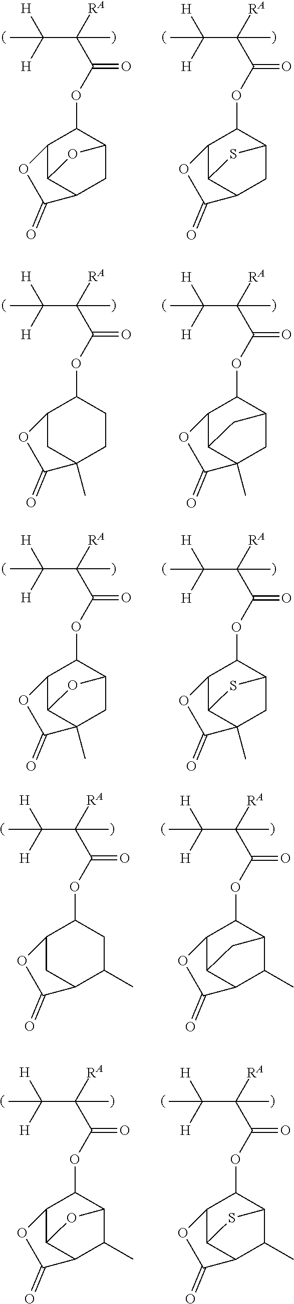

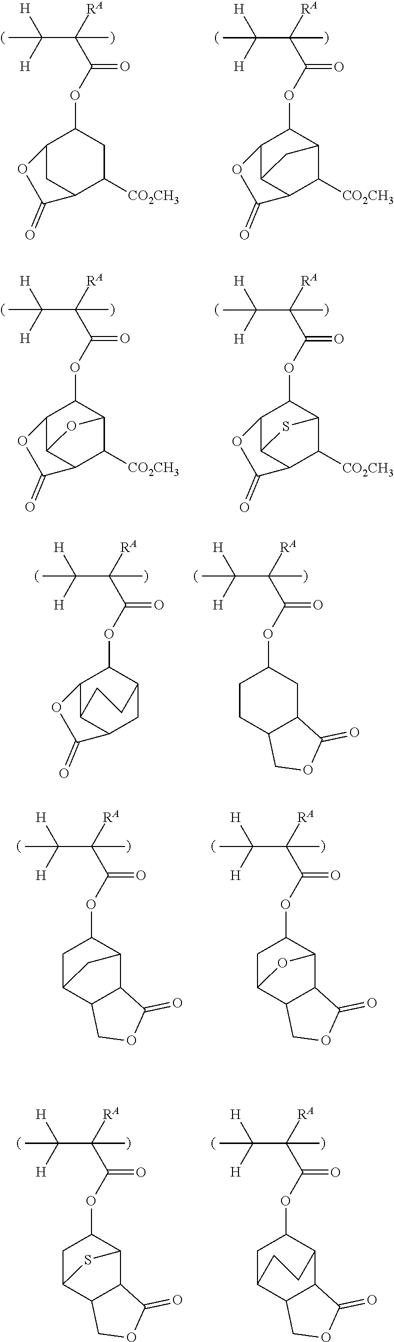

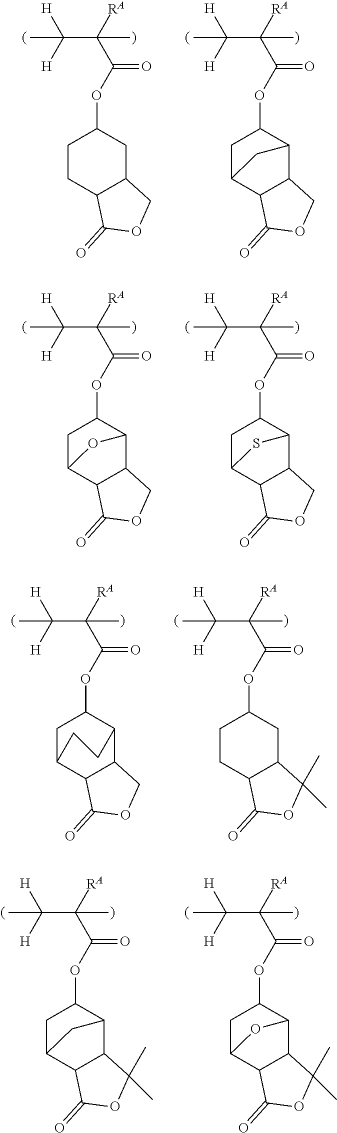

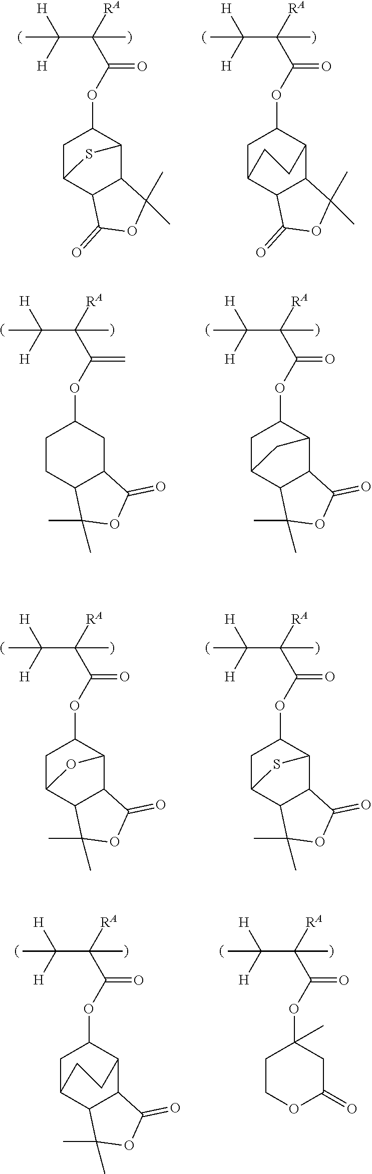

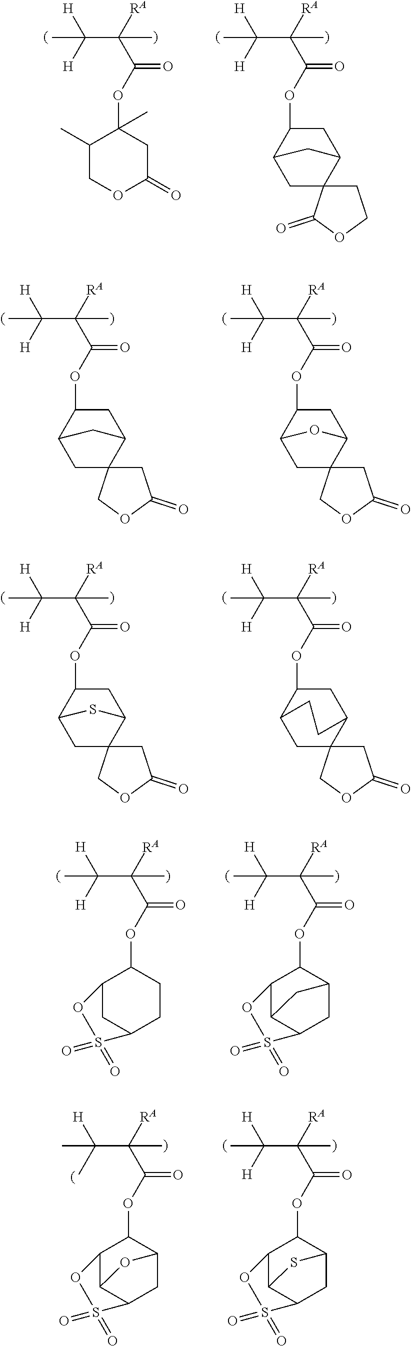

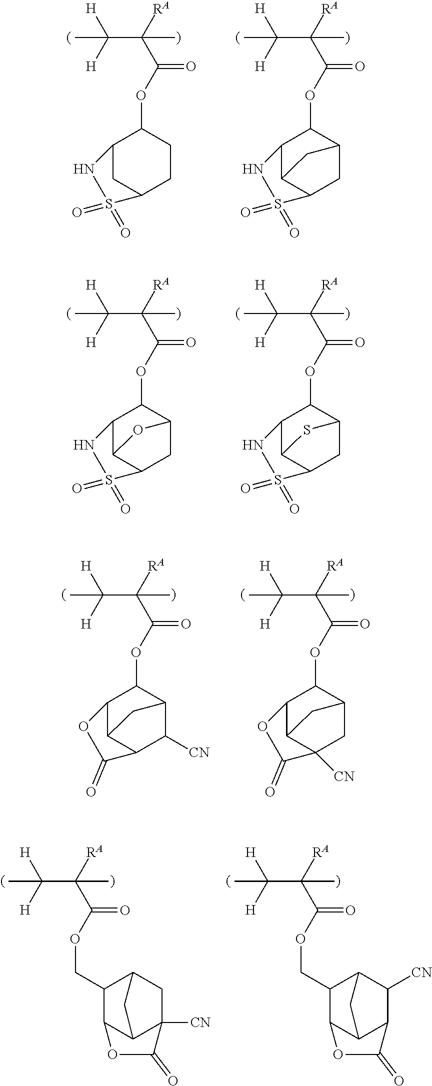

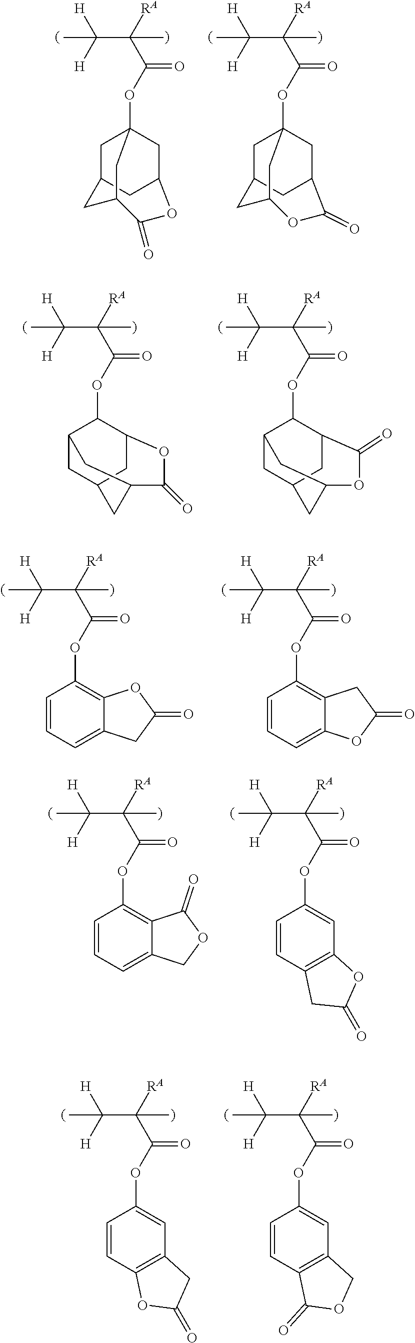

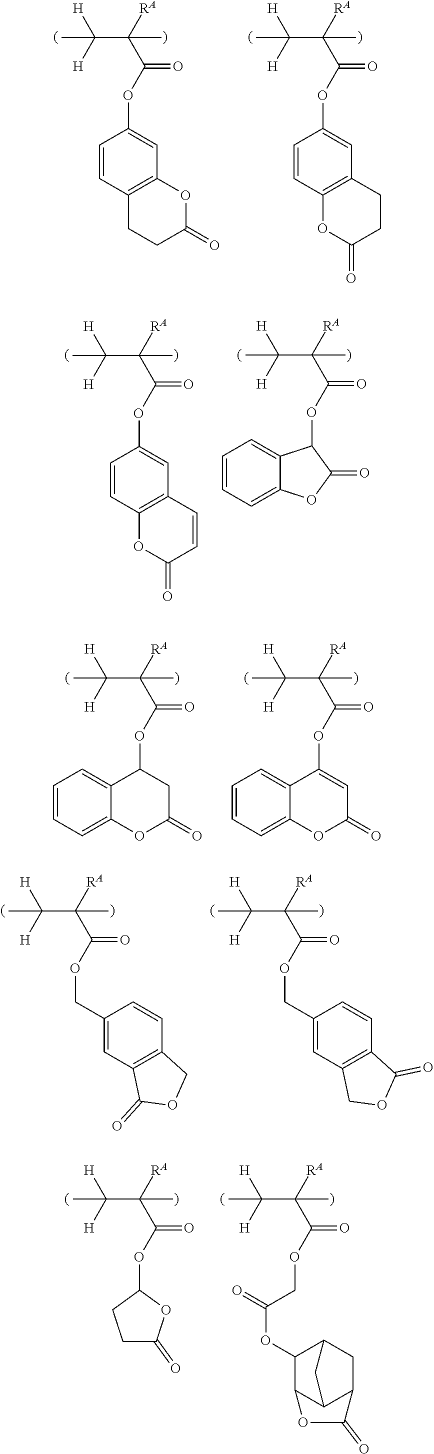

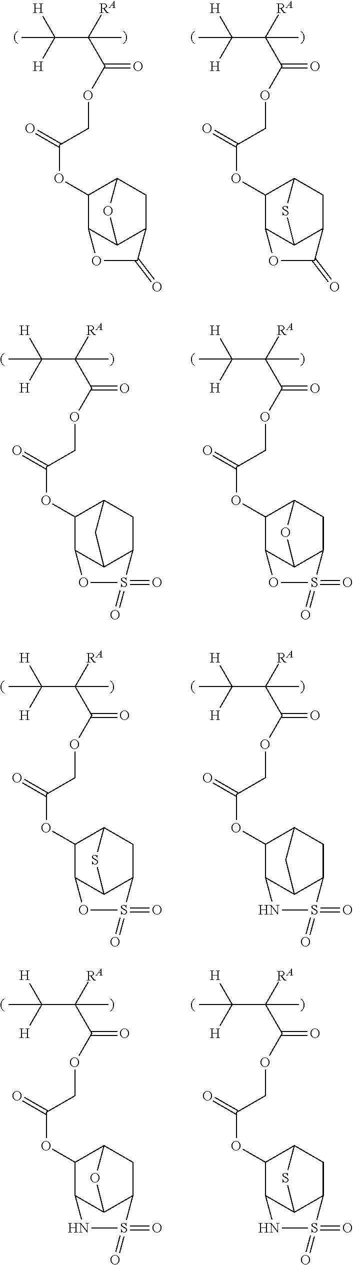

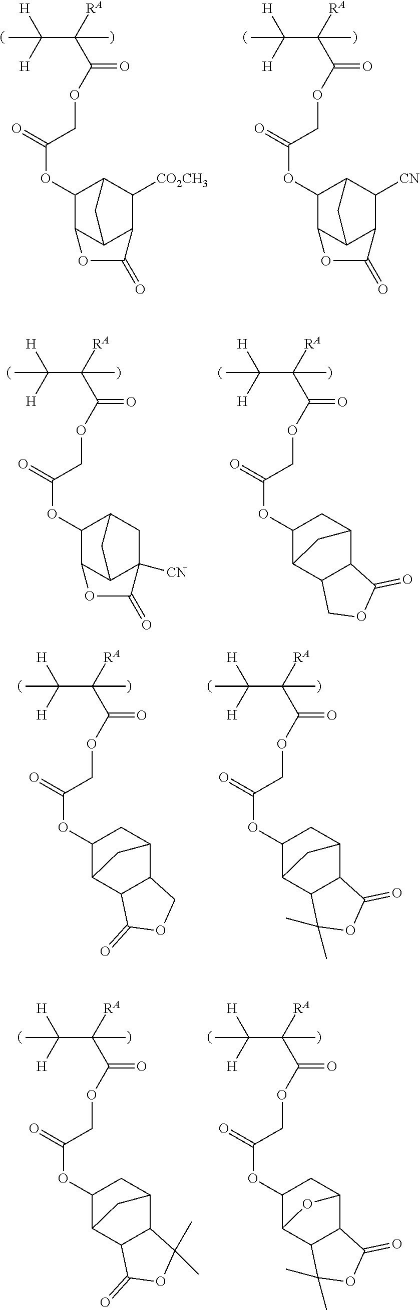

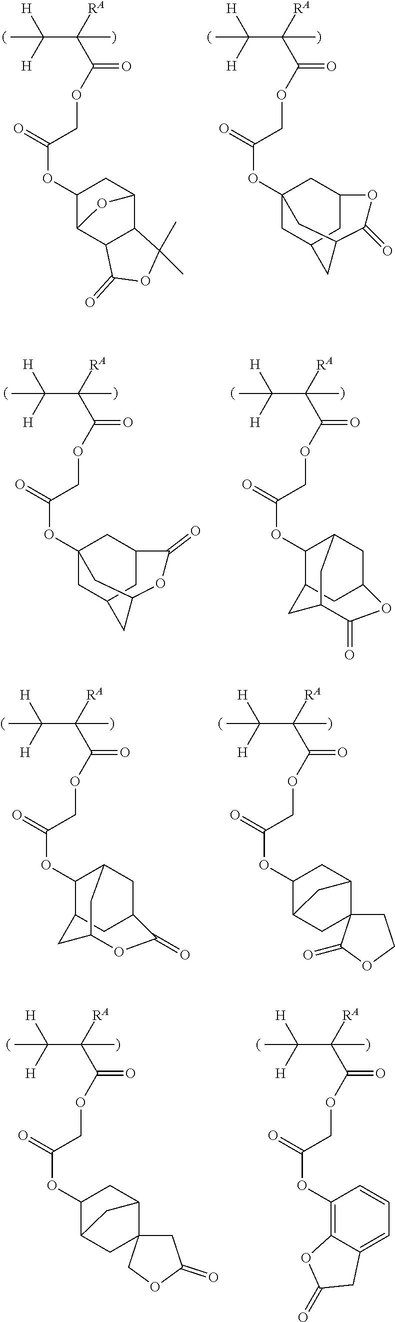

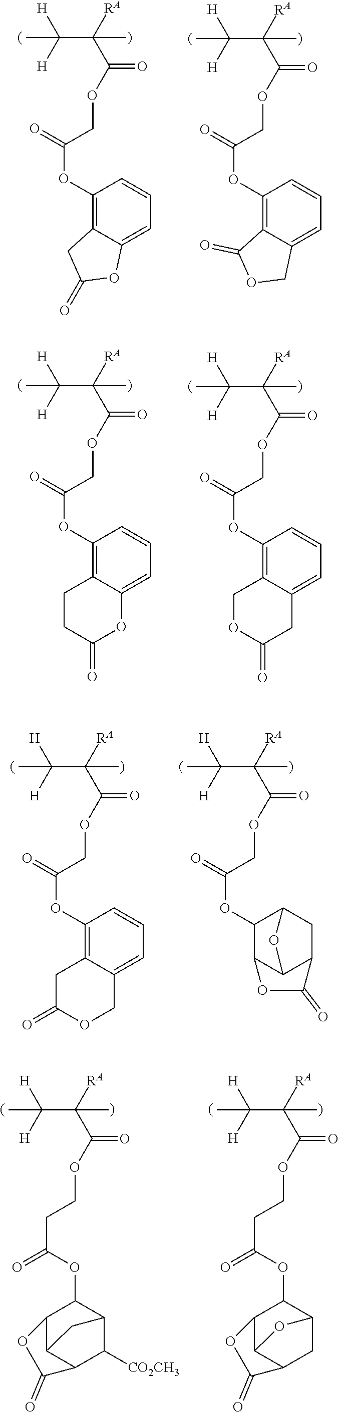

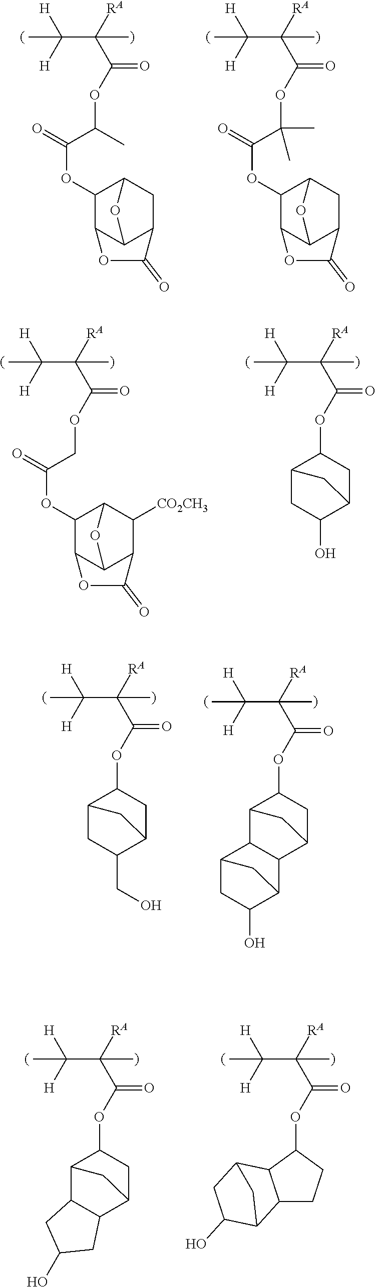

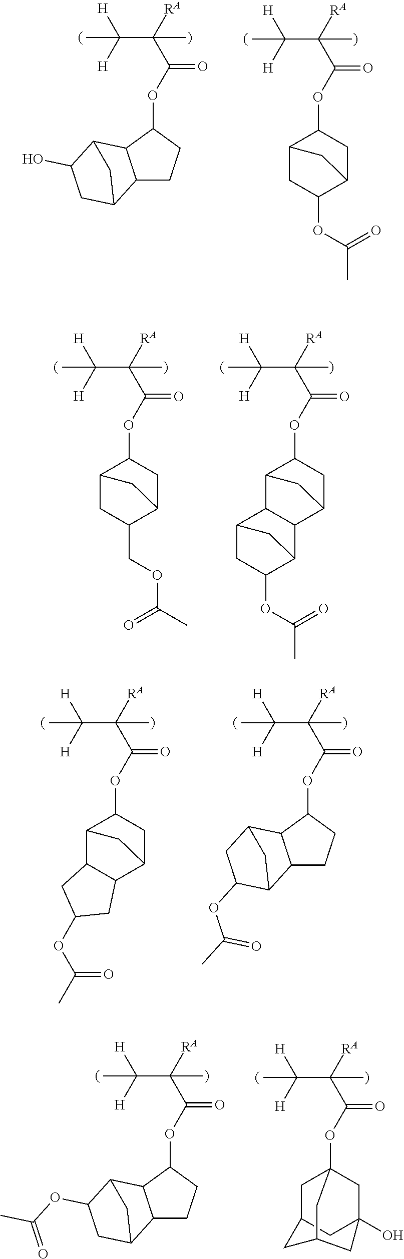

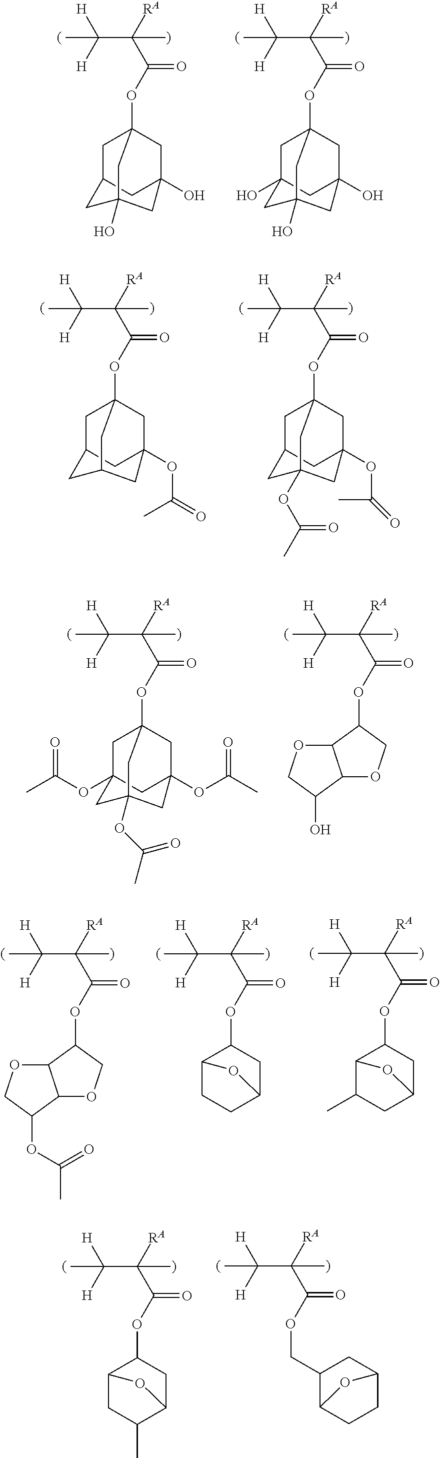

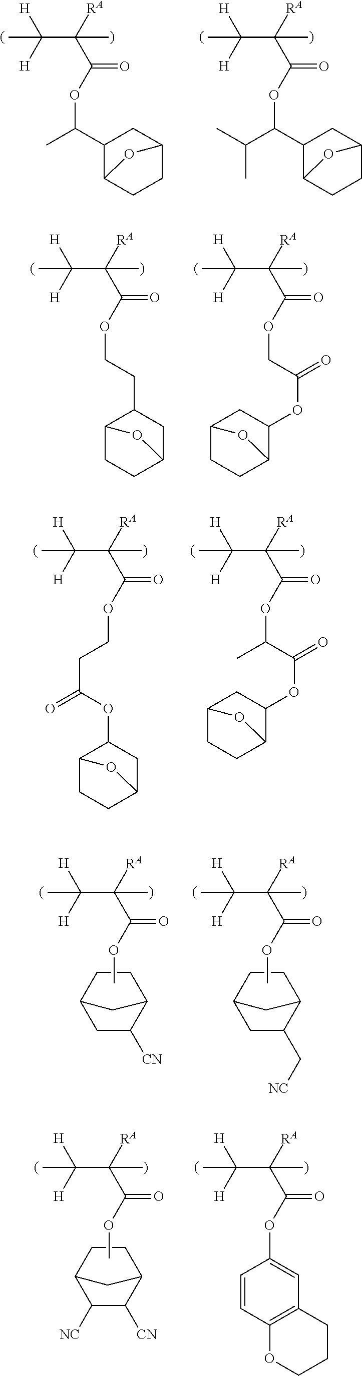

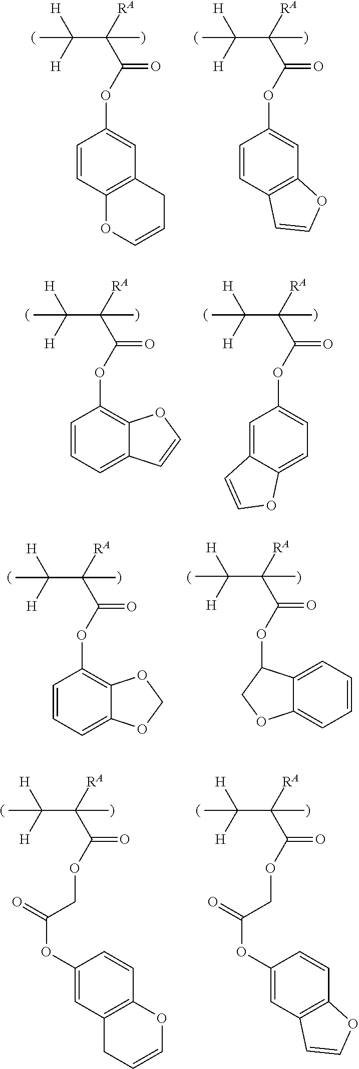

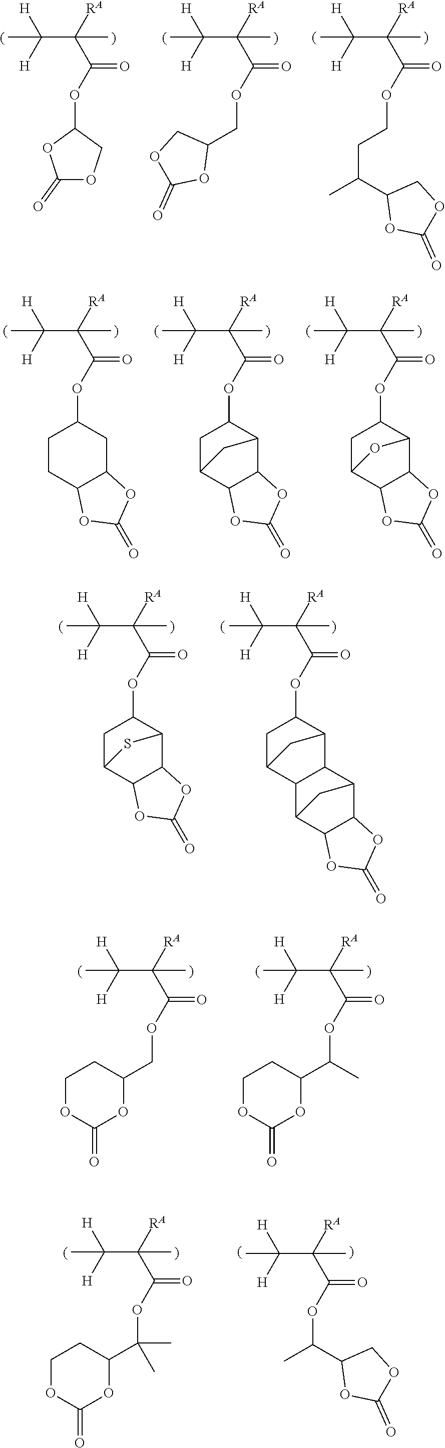

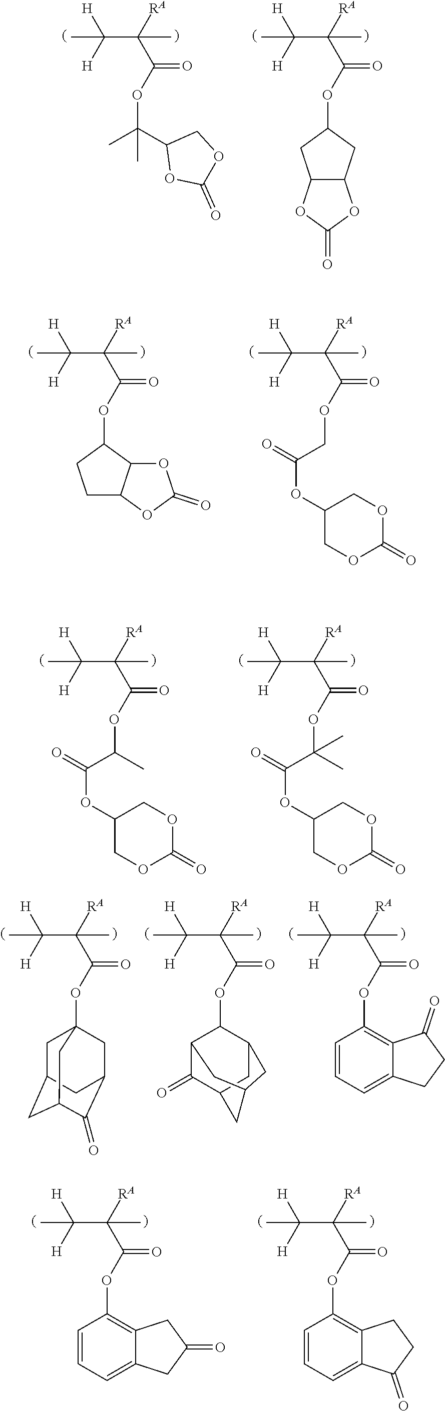

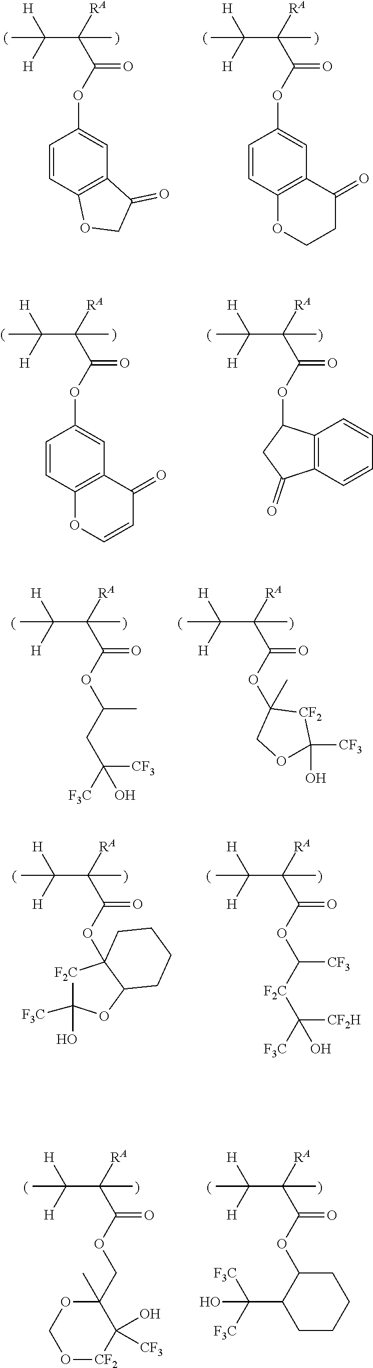

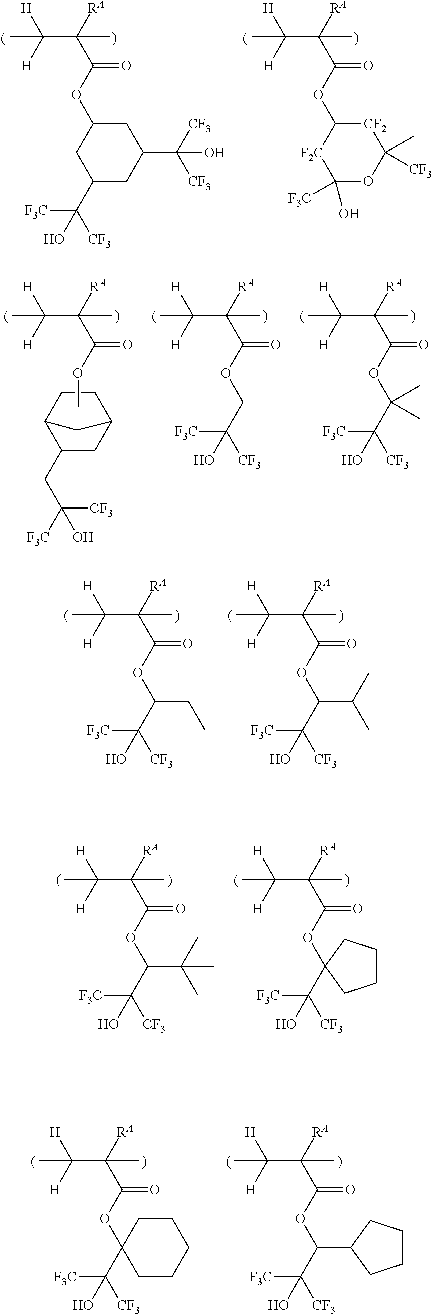

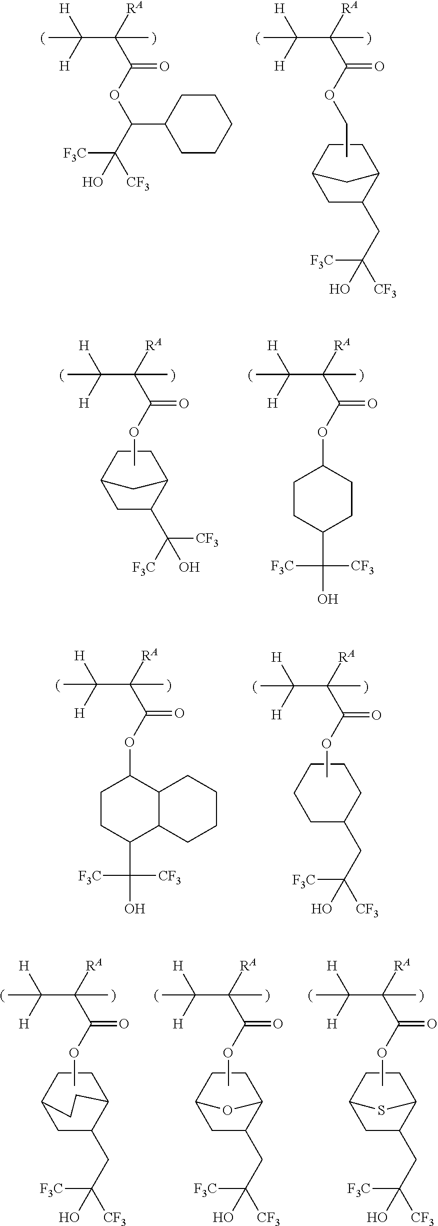

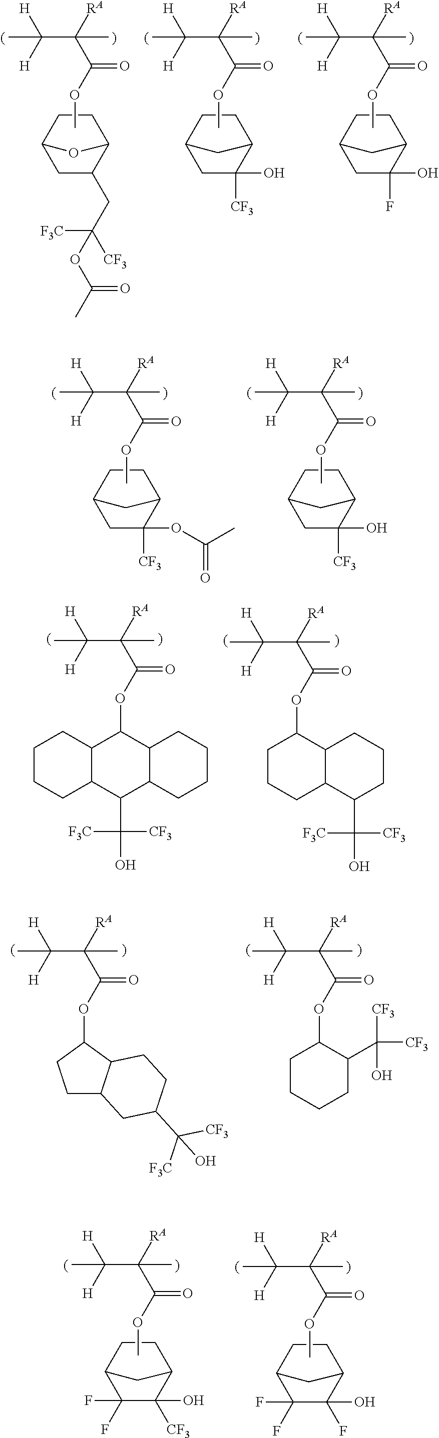

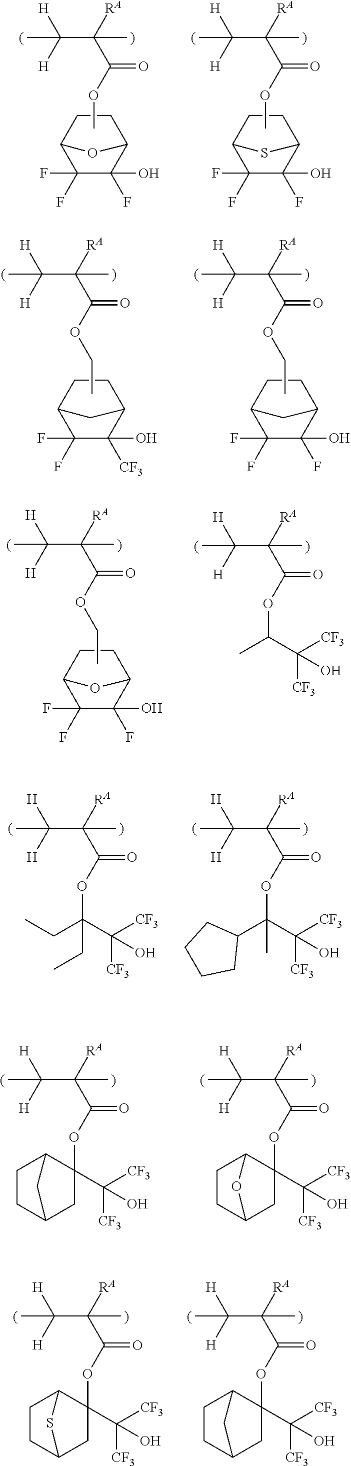

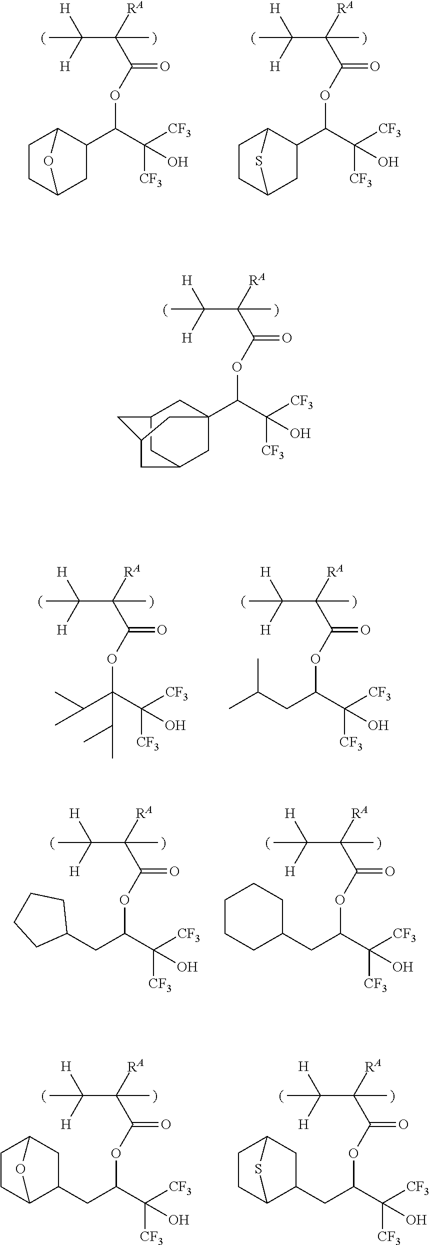

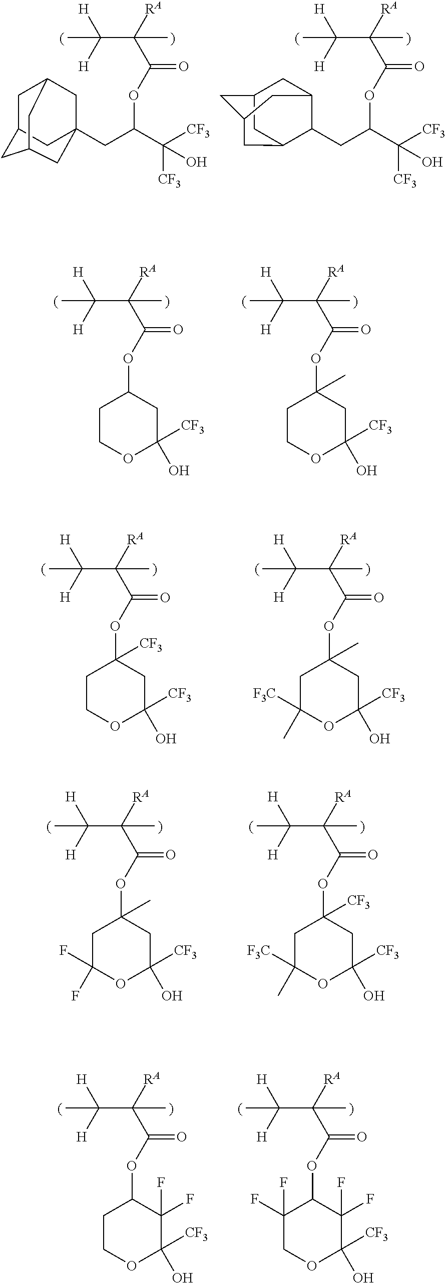

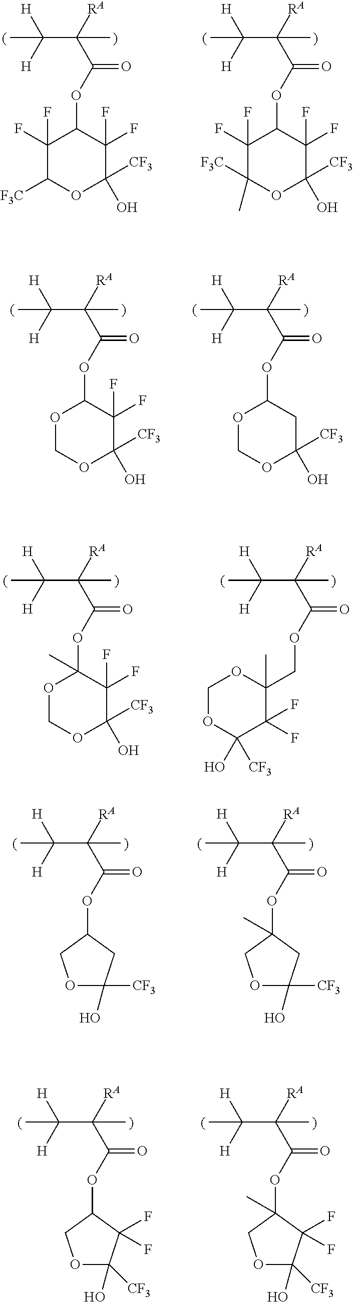

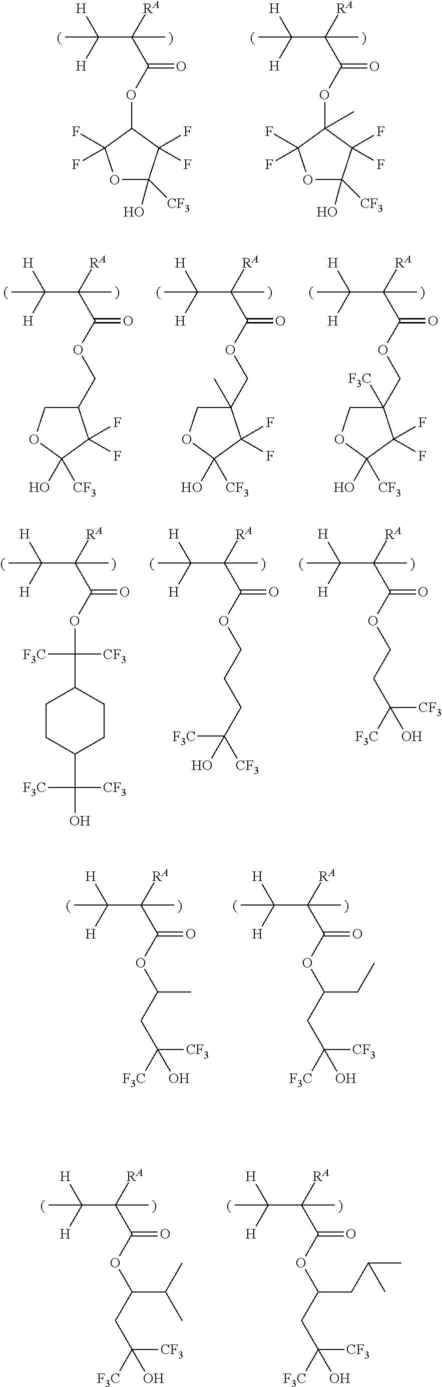

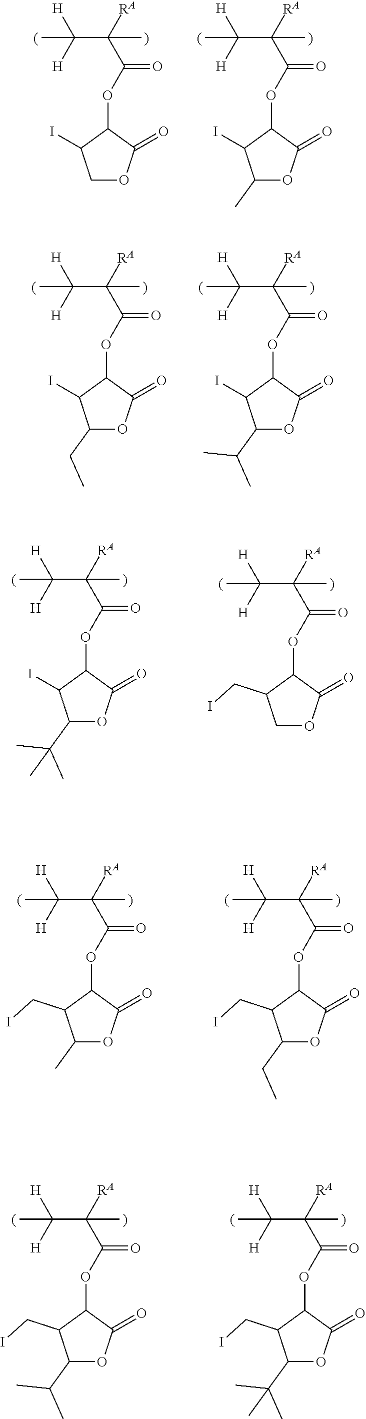

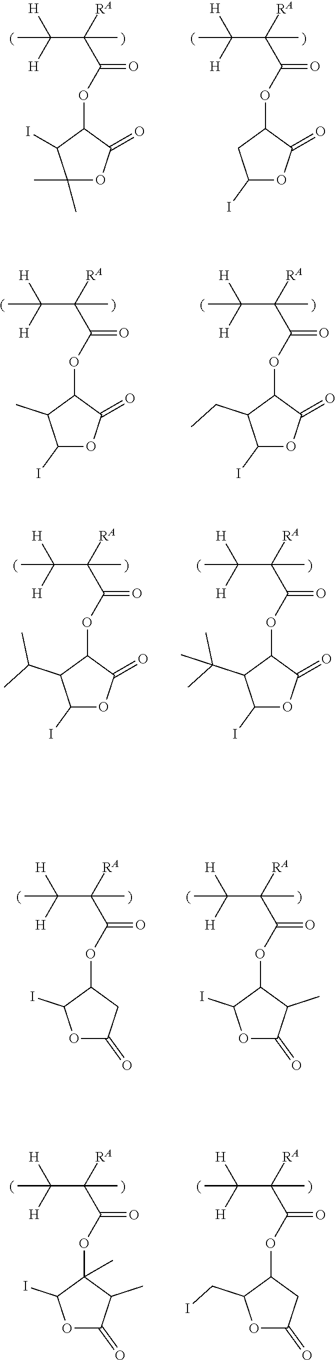

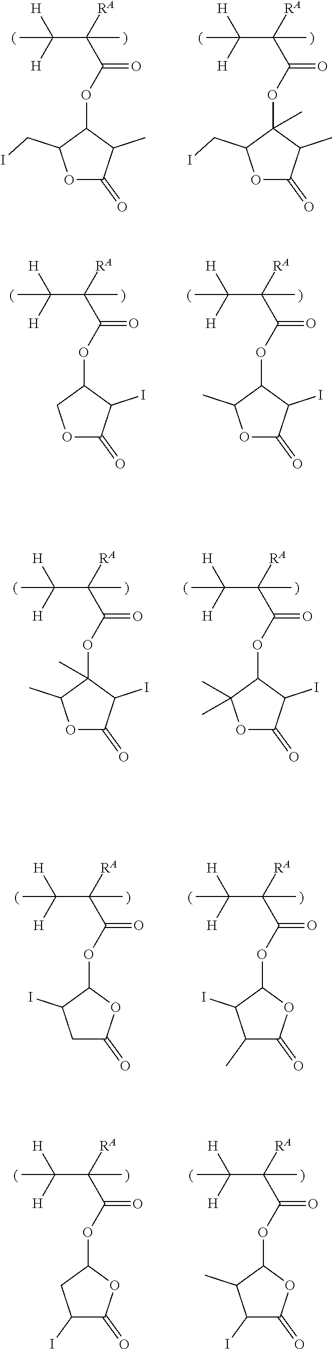

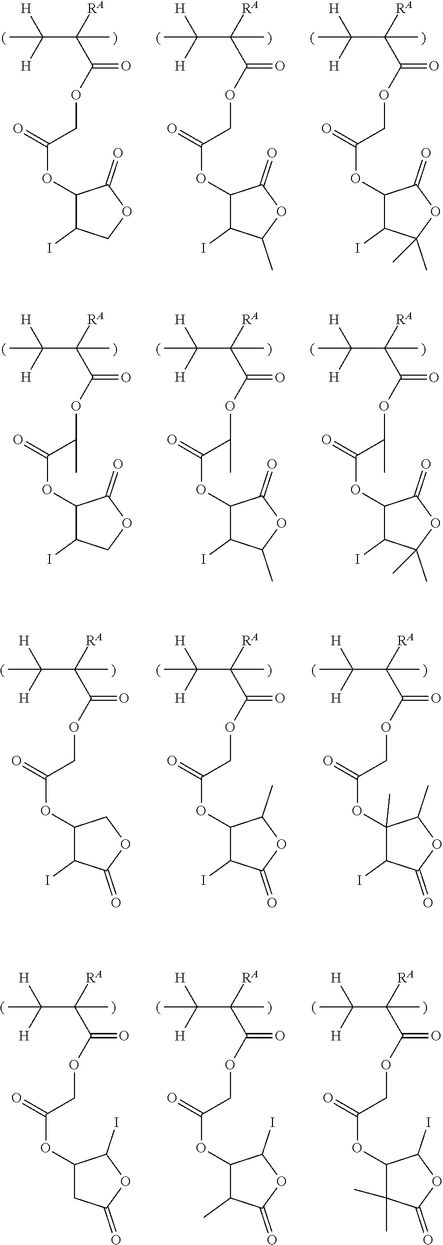

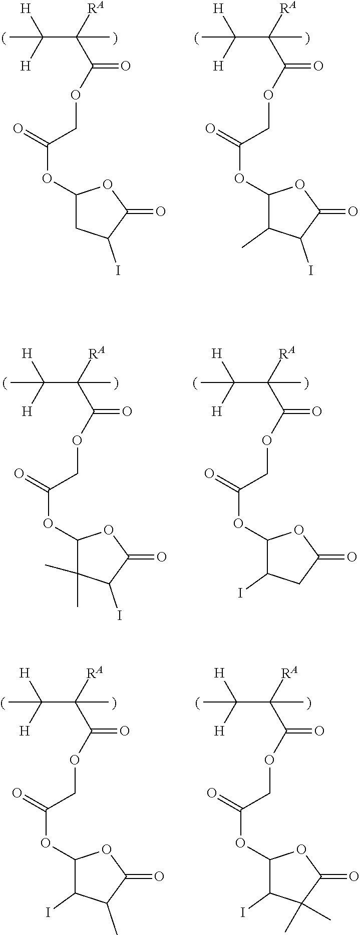

As the repeating unit-d, one having a lactone ring as a polar group is particularly preferable in ArF lithography, and one having a phenol moiety is preferable in KrF lithography, EB lithography, and EUV lithography.

The polymer may further include a repeating unit having a structure where a hydroxy group is protected with an acid-labile group (hereinafter, also referred to as repeating unit-e). The repeating unit-e is not particularly limited as long as it has one or more structures where a hydroxy group is protected, the protecting group is decomposed by the action of an acid to generate a hydroxy group, but a repeating unit represented by the following formula (e) is preferable.

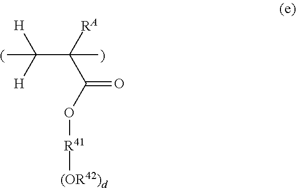

In the formula (e1), R^{A} represents a hydrogen atom, a fluorine atom, a methyl group, or a trifluoromethyl group. R⁴¹ represents a (d+1)-valent hydrocarbon group having 1 to 30 carbon atoms and optionally containing a heteroatom. R⁴² represents an acid-labile group. “d” represents 1, 2, 3, or 4.

In the formula (e), the acid-labile group represented by R⁴² may be deprotected by the action of an acid and generate a hydroxy group. The structure of R⁴² is not particularly limited, but an acetal structure, a ketal structure, an alkoxycarbonyl group, an alkoxymethyl group, etc. are preferable, and an alkoxymethyl group represented by the following formula (e1) is particularly preferable.

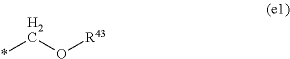

In the formula, “*” represents an attachment point. R⁴³ represents a hydrocarbyl group having 1 to 15 carbon atoms.

Specific examples of the acid-labile group represented by R⁴², the alkoxymethyl group represented by the formula (e1), and the repeating unit-e include those given as examples of the repeating unit-d disclosed in JP 2020-111564 A.

The polymer may further include a repeating unit-f derived from indene, benzofuran, benzothiophene, acenaphthylene, chromone, coumarin, norbornadiene, or a derivative thereof. Specific examples of a monomer to give the repeating unit-f include the following, but are not limited thereto.

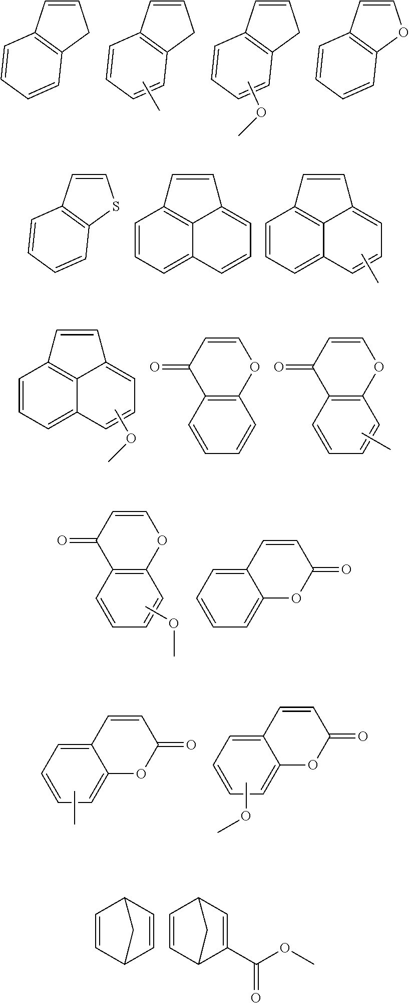

The polymer may further include a repeating unit-g derived from styrene, indane, vinylpyridine, or vinylcarbazole.

In the inventive polymer, the content ratios of the repeating units-a, -b1, -b2, -b3, -c, -d, -e, -f, and -g are preferably 0<a≤0.5, 0≤b1≤0.8, 0≤b2≤0.8, 0≤b3≤0.6, 0≤c≤0.8, 0≤d≤0.5, 0≤e≤0.3, 0≤f≤0.3, and 0≤g≤0.3; more preferably 0<a≤0.4, 0≤b1≤0.7, 0≤b2≤0.7, 0≤b3≤0.5, 0≤c≤0.7, 0≤d≤0.4, 0≤e≤0.2, 0≤f≤0.2, and 0≤g≤0.2, provided that a+b1+b2+b3+c+d+e+f+g≤1.0.

The polymer preferably has a weight-average molecular weight (Mw) of 1000 to 500000, more preferably 3000 to 100000. When the Mw is in these ranges, sufficient etching resistance can be achieved, and there is no risk of resolution being degraded by a difference in dissolution rate between before and after exposure not being ensured. Note that, in the present invention, Mw is a value measured in terms of polystyrene by gel permeation chromatography (GPC) using tetrahydrofuran (THF) or N,N-dimethylformamide (DMF) as an eluent. When measurement by GPC is performed, measurement is usually performed at a room temperature of around 23° C., but may also be performed at a temperature higher or lower than this.

Furthermore, regarding the molecular weight distribution (Mw/Mn) of the polymer, as pattern rule is miniaturized, the influence of Mw/Mn is likely to be greater, and therefore, to obtain a resist composition suitably used for a fine pattern size, the Mw/Mn is preferably 1.0 to 2.0, a narrow dispersity. Within this range, there are few polymers having a low molecular weight or a high molecular weight, and there are no risks of foreign substances being found on the pattern or the pattern shape being degraded after exposure.

Examples of methods for synthesizing the polymer include a method of subjecting the monomers to give the repeating units described above to heat polymerization in an organic solvent to which a radical polymerization initiator has been added.

Specific examples of the organic solvent used in the polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2′-azobis(2-methylpropionate), 1,1′-azobis(1-acetoxy-1-phenylethane), benzoyl peroxide, and lauroyl peroxide. The amount of the initiator to be added is preferably 0.01 to 25 mol % of the total amount of the monomers to be polymerized. The reaction temperature is preferably 50 to 150° C., more preferably 60 to 100° C. The reaction time is preferably 2 to 24 hours, and from the viewpoint of production efficiency, more preferably 2 to 12 hours.

The polymerization initiator may be added to the solution of the monomer and supplied to the reaction vessel, or a solution of the initiator may be prepared separately from the solution of the monomer, and each may be supplied to the reaction vessel independently. There is a possibility that the polymerization reaction may progress due to radicals generated from the initiator during waiting time and an ultra-high molecular weight polymer may be generated, and therefore, from the viewpoint of quality control, it is preferable to prepare each of the monomer solution and the initiator solution independently and add the solutions dropwise. An acid-labile group introduced into the monomer may be used as it is, or may be protected or partially protected after polymerization. Furthermore, to adjust the molecular weight, a known chain transfer agent, such as dodecyl mercaptan and 2-mercaptoethanol may also be used. In this case, the amount of the chain transfer agent to be added is preferably 0.01 to 20 mol % of the total amount of the monomers to be polymerized.

In the case of a monomer containing a hydroxy group, the process may include: substituting the hydroxy group with an acetal group susceptible to deprotection with acid, such as an ethoxyethoxy group, prior to the polymerization; and performing the deprotection with weak acid and water after the polymerization. Alternatively, the process may include: substituting the hydroxy group with an acetyl group, a formyl group, a pivaloyl group, or the like prior to the polymerization; and performing alkaline hydrolysis after the polymerization.

In a case where hydroxystyrene or hydroxyvinylnaphthalene is copolymerized, hydroxystyrene or hydroxyvinylnaphthalene and other monomers may be subjected to heat polymerization in an organic solvent to which a radical polymerization initiator has been added; alternatively, acetoxystyrene or acetoxyvinylnaphthalene may be used, and after the polymerization, the acetoxy group may be deprotected by the alkaline hydrolysis to convert the acetoxystyrene or acetoxyvinylnaphthalene to polyhydroxystyrene or hydroxypolyvinylnaphthalene.

As specific examples of the base in the alkaline hydrolysis, ammonia water, triethylamine, etc. are usable. The reaction temperature is preferably −20 to 100° C., more preferably 0 to 60° C. The reaction time is preferably 0.2 to 100 hours, more preferably 0.5 to 20 hours.

Incidentally, the amount of each monomer in the monomer solution can, for example, be set appropriately to achieve the above-described preferable content ratios of the repeating units.

As for the polymer obtained in the above-described manufacturing method, a reaction solution obtained by the polymerization reaction may be a final product. Alternatively, a powder obtained via a purification step, such as a reprecipitation method in which the polymerization solution is added into a poor solvent to obtain a powder, may be treated as a final product. From the viewpoints of operation efficiency and quality stabilization, the powder obtained in the purification step is preferably dissolved in a solvent to form a polymer solution to be operated as a final product.

Specific examples of the solvent used in this case include solvents described in paragraphs [0144] to [0145] in JP 2008-111103 A, and include: ketones, such as cyclohexanone and methyl-2-n-pentylketone; alcohols, such as 3-methoxybutanol, 3-methyl-3-methoxybutanol, 1-methoxy-2-propanol, and 1-ethoxy-2-propanol; ethers, such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters, such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as GBL; alcohols, such as diacetone alcohol (DAA); alcoholic solvents having a high boiling point, such as diethylene glycol, propylene glycol, glycerin, 1,4-butanediol, and 1,3-butanediol; and mixed solvents thereof.

In the polymer solution, the concentration of the polymer is preferably 0.01 to 30 mass %, more preferably 0.1 to 20 mass %.

The reaction solution and the polymer solution are preferably filtered with a filter. The filtration can remove foreign substances and gel, which may cause defects, and is effective in terms of quality stabilization.

Examples of a material of the filter used for the filtration include fluorocarbon, cellulose, nylon, polyester, and hydrocarbon materials. In the step of filtering the chemically-amplified resist composition, preferable is a filter formed with a fluorocarbon, so-called Teflon (registered trademark), a hydrocarbon, such as polyethylene and polypropylene, or nylon. A pore size of the filter can be appropriately selected depending on target cleanliness, and is preferably 100 nm or smaller, and more preferably 20 nm or smaller. One of these filters may be used alone, or a plurality of these filters may be used in combination. As for the filtration method, the solution may be passed through the filter only once, but the solution is preferably circulated to be filtered a plurality of times. In the step for producing the polymer, the filtration step may be performed in any order any number of times, but the reaction solution after the polymerization reaction, the polymer solution, or both thereof are preferably filtered.

### [Chemically-Amplified Resist Composition]

### [(A) Base Polymer]

The inventive chemically-amplified resist composition contains, as a component (A), a base polymer including the above-described polymer.

One kind of the polymer may be used, or two or more kinds thereof that differ in composition ratio, Mw, and/or Mw/Mn may be used in combination. Furthermore, the base polymer (A) may include, in addition to the above-described polymer, a hydrogenated ring-opening metathesis polymer, and regarding this polymer, those disclosed in JP 2003-66612 A can be used.

The amount of the component (A) contained in the inventive chemically-amplified resist composition is not particularly limited, and can be, for example, 0.1 to 10 parts by mass, preferably 0.5 to 5 parts by mass based on 100 parts by mass of the total composition.

### [(B) Organic Solvent]

The inventive chemically-amplified resist composition may contain an organic solvent as a component (B). The organic solvent (B) is not particularly limited as long as it is capable of dissolving the above-described components and the components described later. Specific examples of such an organic solvent include: ketones, such as cyclopentanone, cyclohexanone, and methyl-2-n-pentyl ketone; alcohols, such as 3-methoxybutanol, 3-methyl-3-methoxybutanol, 1-methoxy-2-propanol, and 1-ethoxy-2-propanol; ketoalcohols, such as DAA; ethers, such as PGME, ethylene glycol monomethyl ether, propylene glycol monoethyl ether, ethylene glycol monoethyl ether, propylene glycol dimethyl ether, and diethylene glycol dimethyl ether; esters, such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones, such as GBL; and mixed solvents thereof.

Among these organic solvents, 1-ethoxy-2-propanol, PGMEA, cyclohexanone, GBL, DAA, and mixed solvents thereof, which are particularly excellent in the solubility of the component (A) base polymer, are preferable.

In the inventive chemically-amplified resist composition, the organic solvent (B) is preferably contained in an amount of 200 to 5000 parts by mass, more preferably 400 to 3500 parts by mass based on 80 parts by mass of the base polymer (A). One kind of the organic solvent (B) may be used, or two or more kinds thereof may be used in mixture.

### [(C) Quencher]

The inventive chemically-amplified resist composition may contain a quencher as a component (C). Note that, in the present invention, a quencher means a material for trapping an acid generated from the photo-acid generator in the chemically-amplified resist composition to prevent diffusion to unexposed portions and forming a desired pattern.

Specific examples of the quencher (C) include onium salts represented by the following formula (1) or (2).

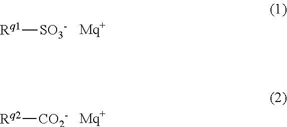

In the formula (1), R^{q1} represents a hydrogen atom or a hydrocarbyl group having 1 to 40 carbon atoms and optionally containing a heteroatom, excluding hydrocarbyl groups in which the hydrogen atom bonded to the carbon atom in the a position of the sulfo group is substituted with a fluorine atom or a fluoroalkyl group. In the formula (2), R^{q2} represents a hydrogen atom or a hydrocarbyl group having 1 to 40 carbon atoms and optionally containing a heteroatom.

Specific examples of the hydrocarbyl group represented by R^{q1} having 1 to 40 carbon atoms include: alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; aryl groups having 6 to 40 carbon atoms, such as a phenyl group, a naphthyl group, and an anthracenyl group; etc. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

Specific examples of the hydrocarbyl group represented by R^{q2} also include, besides the substituents given as specific examples of R^{q1}, fluorinated saturated hydrocarbyl groups, such as a trifluoromethyl group and a trifluoroethyl group, and fluorinated aryl groups, such as a pentafluorophenyl group and a 4-trifluoromethylphenyl group.

Specific examples of the anion of the onium salt represented by the formula (1) include the following, but are not limited thereto.

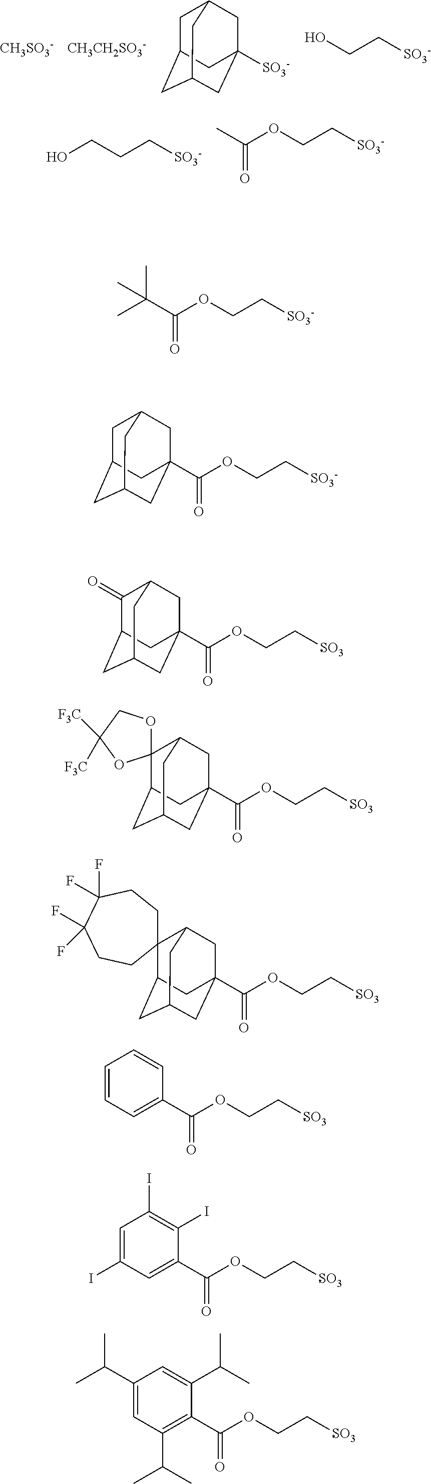

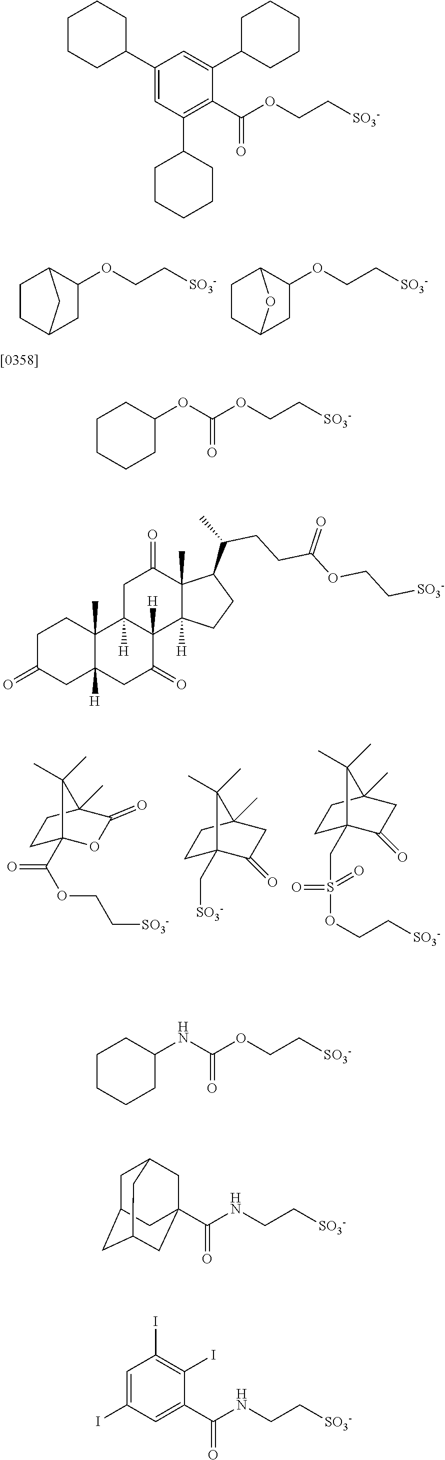

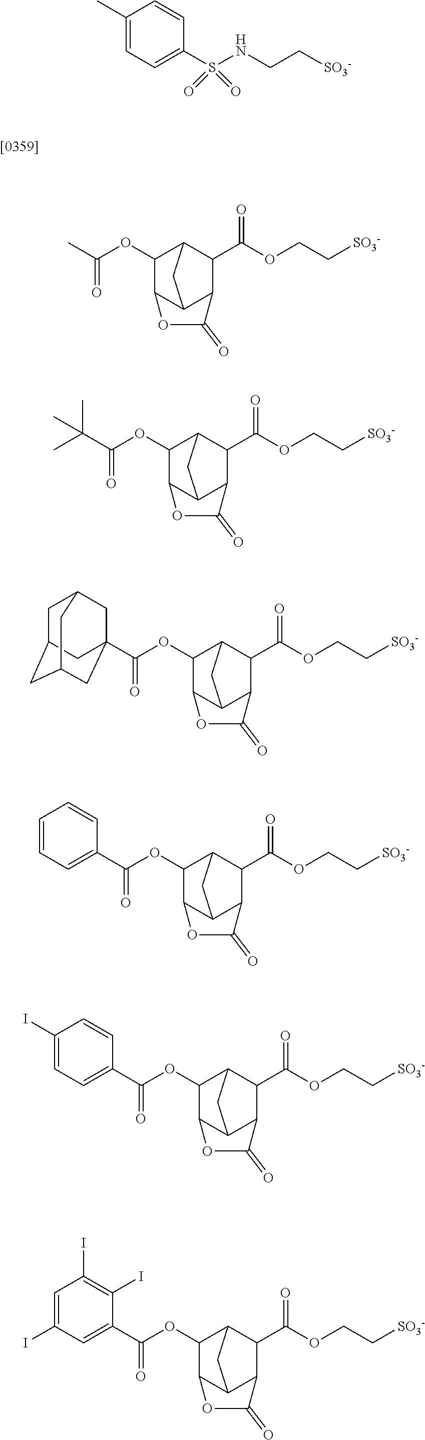

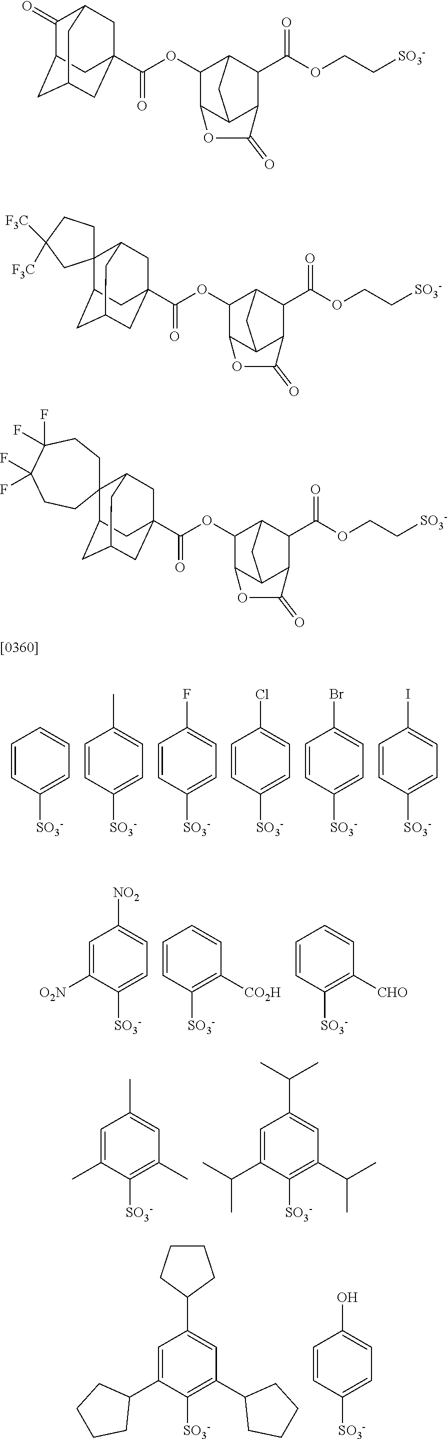

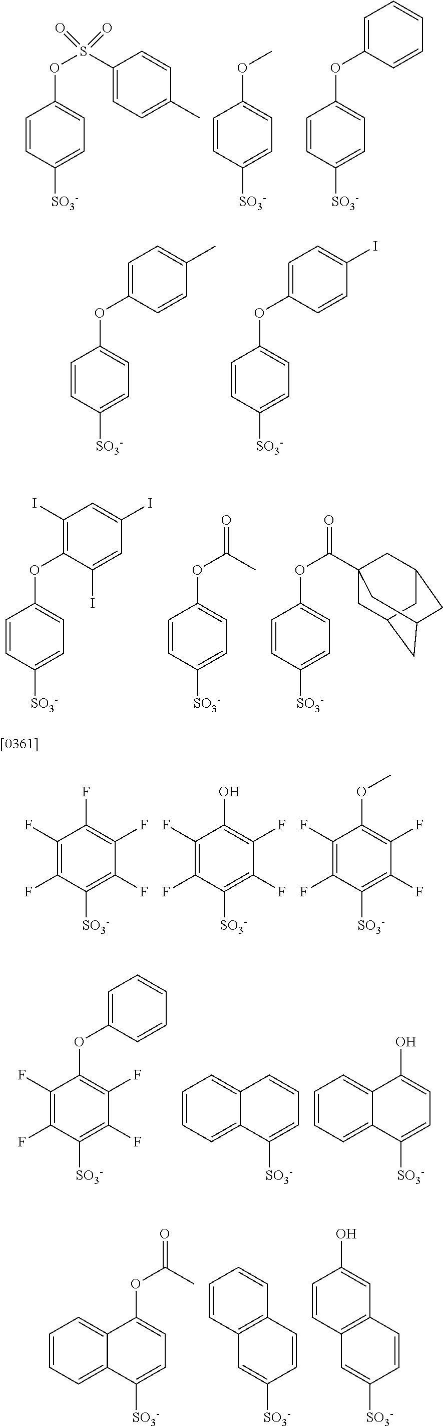

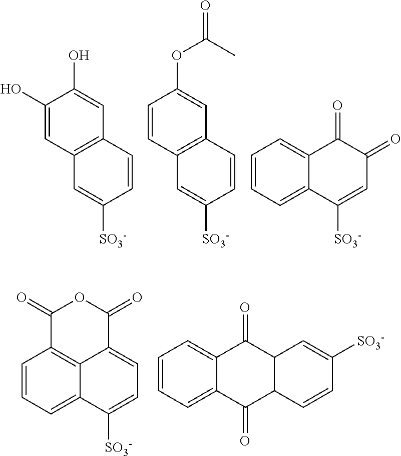

Specific examples of the anion of the onium salt represented by the formula (2) include the following, but are not limited thereto.

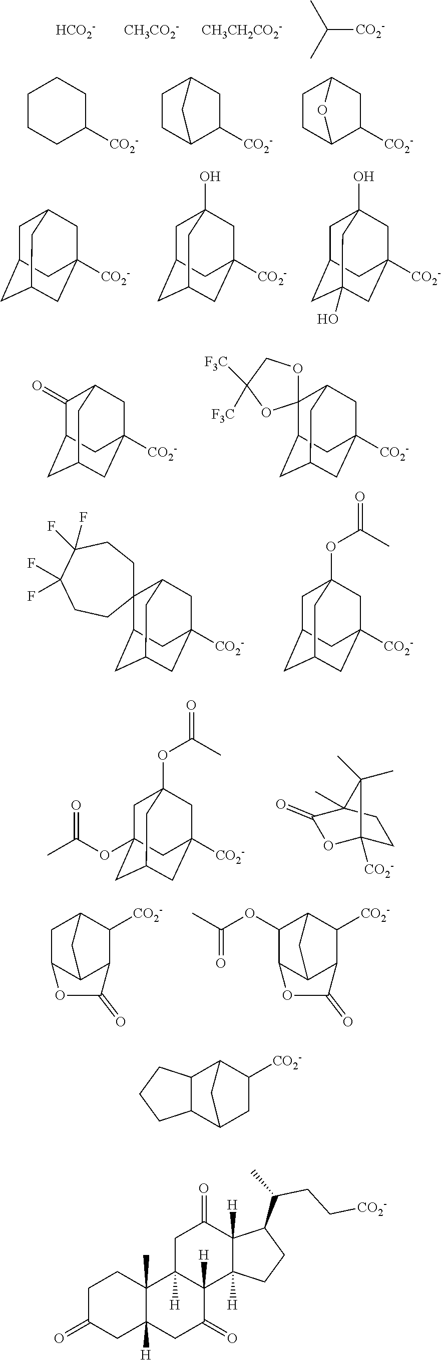

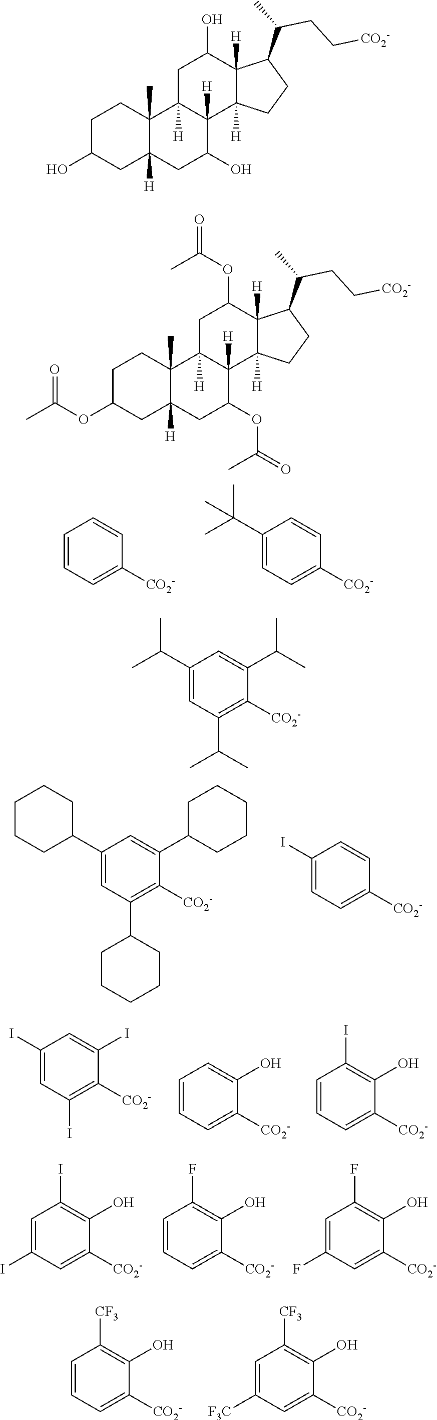

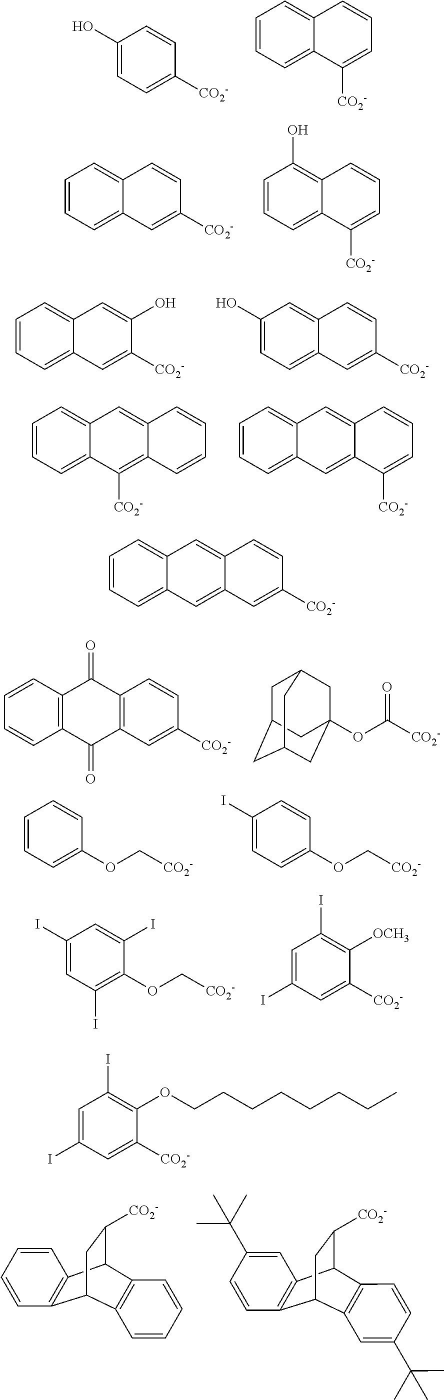

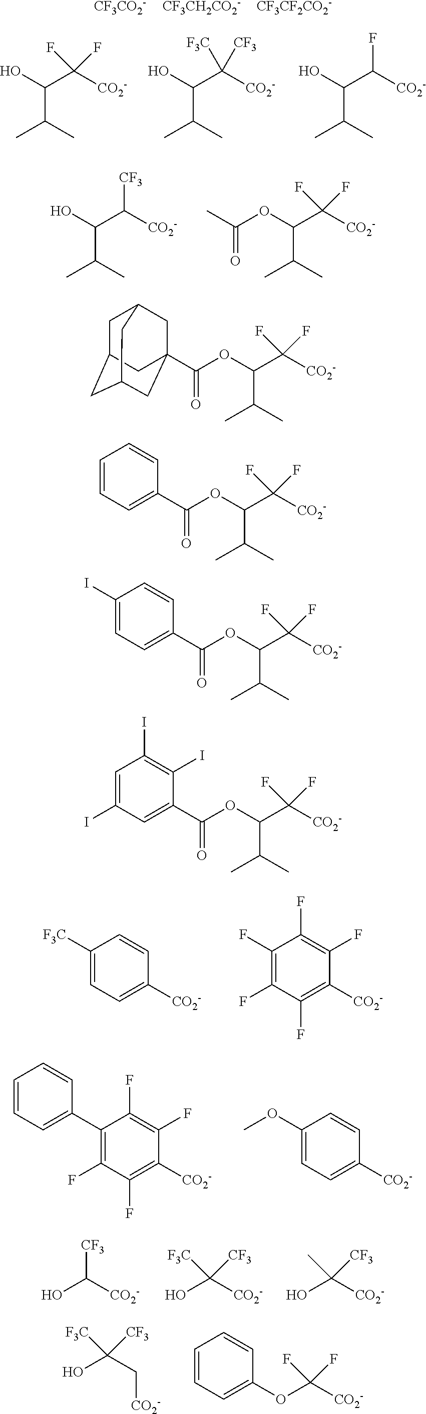

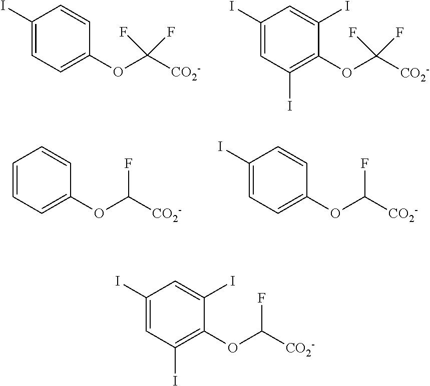

In the formulae (1) and (2), Mq⁺ represents an onium cation. As the onium cation, preferable are the sulfonium cations represented by the above-described formulae (Z-1) and (Z-3), the iodonium cation represented by the above-described formula (Z-2), or the ammonium cation represented by the following formula (am-1).

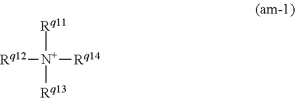

In the formula (am-1), R^{q11} to R^{q14} each independently represent a hydrocarbyl group having 1 to 40 carbon atoms and optionally containing a heteroatom. Furthermore, R^{q11} and R^{q12} may be bonded to each other to form a ring together with the nitrogen atom bonded thereto. Specific examples of the hydrocarbyl groups include those given as examples of the hydrocarbyl groups represented by R^{ct1} to R^{ct5} in the description of the formulae (Z-1) and (Z-2).

Specific examples of the ammonium cation represented by the formula (am-1) include the following, but are not limited thereto.

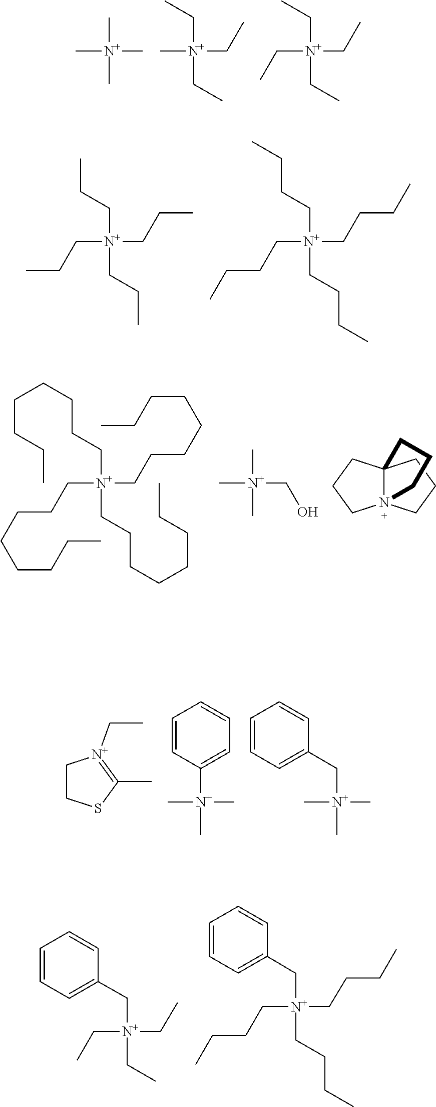

Specific examples of the onium salt represented by the formula (1) or (2) include any combination of the above-described anions and cations. Incidentally, these onium salts can be easily prepared by an ion-exchange reaction using a known organic chemistry process. Regarding the ion-exchange reaction, JP 2007-145797 A may be consulted, for example.

The onium salt represented by the formula (1) or (2) acts as a quencher in the inventive chemically-amplified resist composition. This results from the fact that each counter anion of the onium salts is a conjugate base of a weak acid. Here, weak acid means an acid that exhibits an acidity at which the acid-labile group of the unit, containing an acid-labile group, used in the base polymer cannot be deprotected. The onium salt represented by the formula (1) or (2) functions as a quencher when used in combination with an onium-salt-type photo-acid generator having, as a counter anion, a conjugate base of a strong acid, such as a sulfonic acid fluorinated at the a position. That is, when an onium salt that generates a strong acid, such as a sulfonic acid fluorinated at the a position, and an onium salt that generates a weak acid, such as a sulfonic acid not fluorinated or a carboxylic acid, are used in mixture, collision between a strong acid generated from the photo-acid generator by irradiation with a high-energy beam and an onium salt having an unreacted weak acid anion causes a weak acid to be released by salt exchange, and an onium salt having a strong acid anion is generated. In this process, the strong acid is exchanged with a weak acid having a lower catalytic ability. Therefore, the acid appears to be deactivated, and acid diffusion can be controlled.

Furthermore, as the quencher (C), it is also possible to use: an onium salt disclosed in JP 6848776 B2, having a sulfonium cation and a phenoxide anion moiety within a single molecule; furthermore, an onium salt disclosed in JP 6583136 B2 or JP 2020-200311 A, having a sulfonium cation and a carboxylate anion moiety within a single molecule; or an onium salt disclosed in JP 6274755 B2, having an iodonium cation and a carboxylate anion moiety within a single molecule.

Here, in a case where the photo-acid generator that generates a strong acid is an onium salt, as described above, it is possible to exchange, with a weak acid, a strong acid generated by irradiation with a high-energy beam. However, on the other hand, it is assumed that it is difficult for a weak acid generated by irradiation with a high-energy beam to collide with an unreacted onium salt that generates a strong acid and undergo salt exchange. This results from the phenomenon that an onium cation forms an ion pair more easily with an anion of a stronger acid.

When the inventive chemically-amplified resist composition contains, as the quencher (C), an onium salt represented by the formula (1) or (2), the contained amount is preferably 0.1 to 20 parts by mass, more preferably 0.1 to 10 parts by mass based on 80 parts by mass of the base polymer (A). When the contained amount of the onium-salt-type quencher of the component (C) is in such ranges, resolution is favorable, and there is no remarkable degradation of sensitivity. Therefore, such ranges are preferable. One kind of the onium salt represented by the formula (1) or (2) may be use, or two or more kinds thereof may be used in combination.

The inventive chemically-amplified resist composition may contain a nitrogen-containing compound as the quencher (C). Specific examples of the nitrogen-containing compound of the component (C) include primary, secondary, and tertiary amine compounds disclosed in paragraphs [0146] to [0164] of JP 2008-111103 A; in particular, amine compounds having a hydroxy group, an ether bond, an ester bond, a lactone ring, a cyano group, or a sulfonic acid ester bond. Furthermore, examples also include compounds in which a primary or secondary amine is protected with a carbamate group, disclosed in JP 3790649 B2. Further examples include compounds having an acid-labile group within the molecule, disclosed in JP 2009-109595 A.

It is also possible to use, as a nitrogen-containing compound, a sulfonic acid sulfonium salt having a nitrogen-containing substituent. Such a compound functions as a so-called photo-degradable base, which functions as a quencher in unexposed portions and loses the quencher function in exposed portions due to neutralization with the acid generated by itself. Using a photo-degradable base, the contrast between exposed and unexposed portions can be further enhanced. Regarding the photo-degradable base, JP 2009-109595 A, JP 2012-46501 A, etc. may be consulted, for example.

When the inventive chemically-amplified resist composition contains a nitrogen-containing compound as the quencher (C), the contained amount is preferably 0.001 to 12 parts by mass, more preferably 0.01 to 8 parts by mass based on 80 parts by mass of the base polymer (A). One kind of the nitrogen-containing compound may be used, or two or more kinds thereof may be used in combination.

### [(D) Acid Generator]

The inventive chemically-amplified resist composition may contain an acid generator. Examples of the acid generator include compounds that generate acids in response to actinic light or radiation (photo-acid generators). The photo-acid generator is not particularly limited as long as the compound generates an acid upon high-energy beam irradiation. Preferably, the photo-acid generator generates a sulfonic acid, imide acid, or methide acid. Suitable photo-acid generators include sulfonium salt, iodonium salt, sulfonyldiazomethane, N-sulfonyloxyimide, oxime-O-sulfonate type acid generators, etc. Specific examples of the acid generator include ones disclosed in paragraphs [0122] to [0142] of JP 2008-111103 A.

Moreover, a sulfonium salt represented by the following formula (3-1) and an iodonium salt represented by the following formula (3-2) can also be used suitably as photo-acid generators.

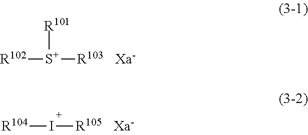

In the formulae (3-1) and (3-2), R¹⁰¹ to R¹⁰⁵ each independently represent a halogen atom or a hydrocarbyl group having 1 to 20 carbon atoms and optionally containing a heteroatom. Specific examples of the halogen atom and the hydrocarbyl group include those given as examples of the halogen atom and the hydrocarbyl group represented by R^{ct1} to R^{ct5} in the description of the formulae (Z-1) and (Z-2). Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a nitro group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc. Furthermore, R¹⁰¹ and R¹⁰² may be bonded to each other to form a ring together with the sulfur atom bonded thereto. Specific examples of the ring formed in this case include those given as examples of the ring that may be formed by R^{ct1} and R^{ct2} being bonded to each other together with the sulfur atom bonded thereto in the description of the formula (Z-1).

Specific examples of the cation of the sulfonium salt represented by the formula (3-1) include those given as examples of the sulfonium cations represented by the formulae (Z-1) and (Z-3). Meanwhile, specific examples of the cation of the iodonium salt represented by the formula (3-2) include those given as examples of the iodonium cation represented by the formula (Z-2).

In the formulae (3-1) and (3-2), Xa⁻ represents an anion selected from the following formulae (3A) to (3D).

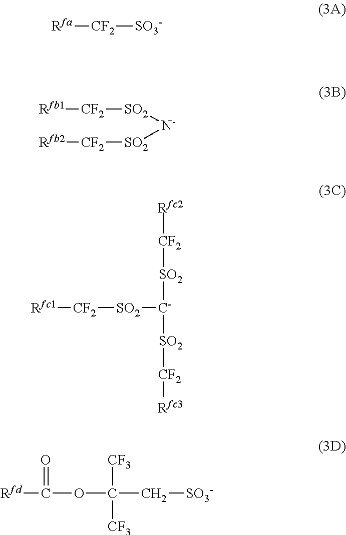

In the formula (3A), R^{f}a represents a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given later in the description of R^{fa1} of the formula (3A′).

As the anion represented by the formula (3A), one represented by the following formula (3A′) is preferable.

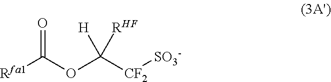

In the formula (3A′), R^{HF} represents a hydrogen atom or a trifluoromethyl group, preferably a trifluoromethyl group.

In the formula (3A′), R^{fa1} represents a hydrocarbyl group having 1 to 38 carbon atoms and optionally containing a heteroatom. The heteroatom is preferably an oxygen atom, a nitrogen atom, a sulfur atom, a halogen atom, or the like, more preferably an oxygen atom. The hydrocarbyl group particularly preferably has 6 to 30 carbon atoms from the viewpoint of achieving high resolution in fine pattern formation.

The hydrocarbyl group represented by R^{fa1} having 1 to 38 carbon atoms may be saturated or unsaturated, and may be linear, branched, or cyclic. Specific examples thereof include: alkyl groups having 1 to 38 carbon atoms, such as a methyl group, an ethyl group, an n-propyl group, an isopropyl group, an n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; cyclic saturated hydrocarbyl groups having 3 to 38 carbon atoms, such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; unsaturated aliphatic hydrocarbyl groups having 2 to 38 carbon atoms, such as an allyl group and a 3-cyclohexenyl group; aryl groups having 6 to 38 carbon atoms, such as a phenyl group, a 1-naphthyl group, and a 2-naphthyl group; aralkyl groups having 7 to 38 carbon atoms, such as a benzyl group and a diphenylmethyl group; and groups which are combinations of the groups.

Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a fluorine atom, a chlorine atom, a bromine atom, an iodine atom, a cyano group, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc. Incidentally, an oxygen atom is preferable as the heteroatom. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, a 3-oxocyclohexyl group, etc.

The synthesis of the sulfonium salt containing the anion represented by the formula (3A′) is described in detail in JP 2007-145797 A, JP 2008-106045 A, JP 2009-7327 A, JP 2009-258695 A, etc. In addition, sulfonium salts disclosed in JP 2010-215608 A, JP 2012-41320 A, JP 2012-106986 A, JP 2012-153644 A, etc. are also suitably used.

Specific examples of the anion represented by the formula (3A) include the following, but are not limited thereto. Note that, in the following formulae, Ac represents an acetyl group.

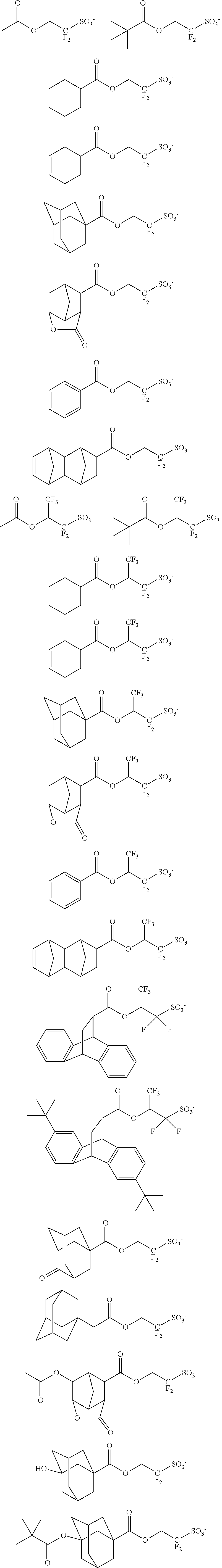

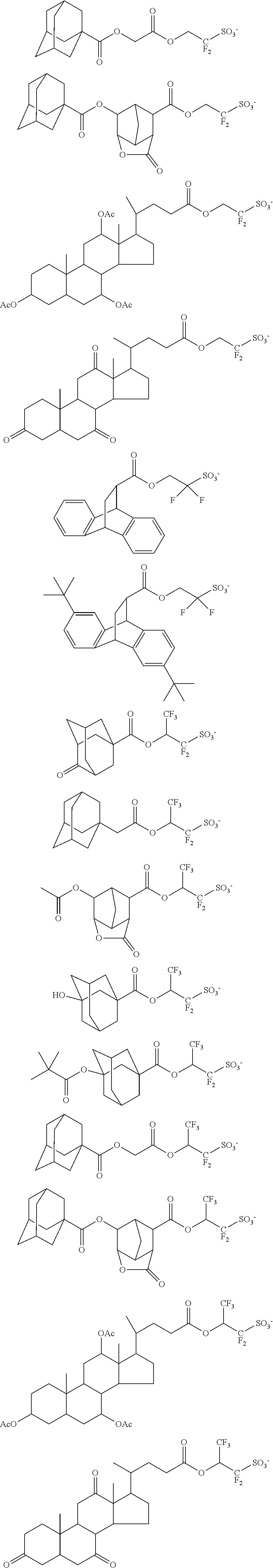

In the formula (3B), R^{fb1} and R^{fb2} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the formula (3A′). R^{fb1} and R^{fb2} are preferably a fluorine atom or a linear fluorinated alkyl group having 1 to 4 carbon atoms. Alternatively, R^{fb1} and R^{fb2} may bond with each other to form a ring together with the group (—CF₂—SO₂—N—SO₂—CF₂—) bonded thereto. In this event, the group obtained by R^{fb1} and R^{fb2} being bonded to each other is preferably a fluorinated ethylene group or a fluorinated propylene group.

In the formula (3C), R^{fc1}, R^{fc2}, and R^{fc3} each independently represent a fluorine atom or a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the formula (3A′). R^{fc1}, R^{fc2}, and R^{fc3} are preferably a fluorine atom or a linear fluorinated alkyl group having 1 to 4 carbon atoms. Alternatively, R^{fc1} and R^{fc2} may bond with each other to form a ring together with the group (—CF₂—SO₂—C—SO₂—CF₂—) bonded thereto. In this event, the group obtained by R^{fc1} and R^{fc2} being bonded to each other is preferably a fluorinated ethylene group or a fluorinated propylene group.

In the general formula (3D), R^{fd} represents a hydrocarbyl group having 1 to 40 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the formula (3A′).

The synthesis of the sulfonium salt containing the anion represented by the formula (3D) is described in detail in JP 2010-215608 A and JP 2014-133723 A.

Specific examples of the anion represented by the formula (3D) include the following, but are not limited thereto.

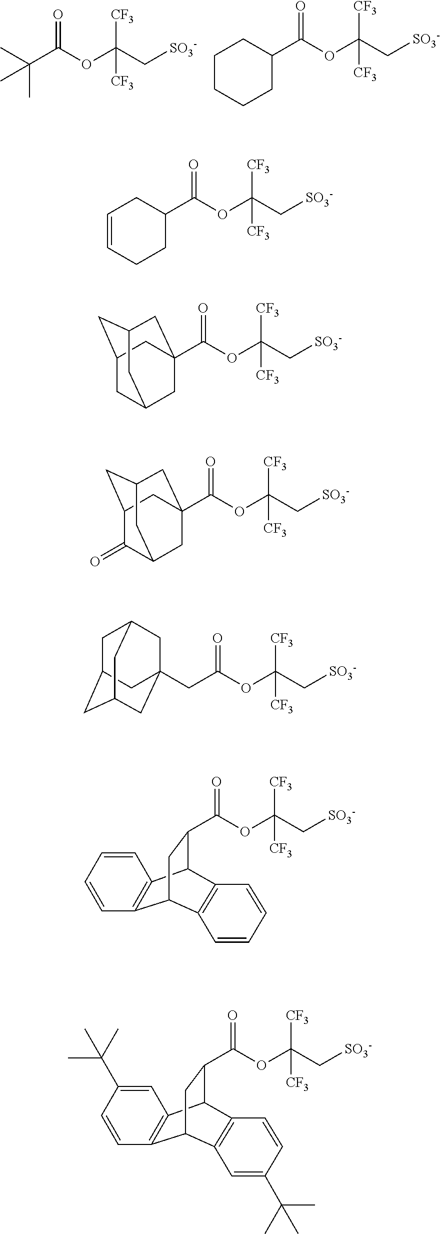

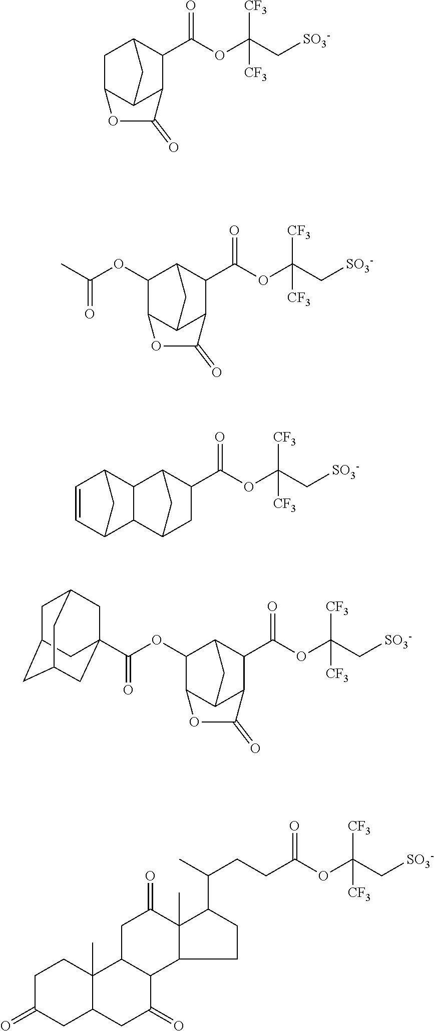

Note that the photo-acid generator containing the anion represented by the formula (3D) does not have a fluorine atom at the a position of the sulfo group, but has two trifluoromethyl groups at the B position, thereby providing sufficient acidity to cut the acid-labile group in the base polymer. Thus, this photo-acid generator is utilizable.

As the photo-acid generator, one represented by the following formula (4) can also be used suitably.

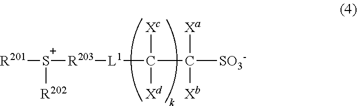

In the formula (4), R²⁰¹ and R²⁰² each independently represent a hydrocarbyl group having 1 to 30 carbon atoms and optionally containing a heteroatom. R²⁰³ represents a hydrocarbylene group having 1 to 30 carbon atoms and optionally containing a heteroatom. In addition, any two of R²⁰¹, R²⁰², and R²⁰³ may be bonded to each other to form a ring together with a sulfur atom bonded thereto. In this event, specific examples of the ring include those given as examples of the ring that may be formed by R^{ct1} and R^{ct2} being bonded to each other together with the sulfur atom bonded thereto in the description of the formula (Z-1).

The hydrocarbyl groups represented by R²⁰¹ and R²⁰² having 1 to 30 carbon atoms may be saturated or unsaturated, and may be linear, branched, or cyclic. Specific examples thereof include: alkyl groups having 1 to 30 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 30 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; aryl groups having 6 to 30 carbon atoms, such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and groups which are combinations of the groups. Furthermore, part or all of the hydrogen atoms of the hydrocarbyl groups may be substituted with a group containing a heteroatom, such as an oxygen atom, a sulfur atom, a nitrogen atom, or a halogen atom, and part of the —CH₂— of the hydrocarbyl groups may be substituted with a group containing a heteroatom such as an oxygen atom, a sulfur atom, or a nitrogen atom. The resulting hydrocarbyl groups may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc.

The hydrocarbylene group represented by R²⁰³ having 1 to 30 carbon atoms may be saturated or unsaturated, and may be linear, branched, or cyclic. Specific examples thereof include alkanediyl groups having 1 to 30 carbon atoms, such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; cyclic saturated hydrocarbylene groups having 3 to 30 carbon atoms, such as a cyclopentanediyl group, a cyclohexanediyl group, a norbornanediyl group, and an adamantanediyl group; arylene groups having 6 to 30 carbon atoms, such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group; and groups which are combinations of the groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— of the hydrocarbylene group may be substituted with a group containing a heteroatom such as an oxygen atom, a sulfur atom, or a nitrogen atom. The resulting hydrocarbylene group may contain a hydroxy group, a cyano group, a fluorine atom, a chlorine atom, a bromine atom, an iodine atom, a carbonyl group, an ether bond, an ester bond, a sulfonic acid ester bond, a carbonate bond, a lactone ring, a sultone ring, a carboxylic anhydride (—C(═O)—O—C(═O)—), a haloalkyl group, etc. As the heteroatom, an oxygen atom is preferable.

In the formula (4), L¹ represents a single bond, an ether bond, or a hydrocarbylene group having 1 to 20 carbon atoms and optionally containing a heteroatom. The hydrocarbylene group may be saturated or unsaturated, and may be linear, branched, or cyclic. Specific examples thereof include those given as examples of the hydrocarbylene group represented by R²⁰³.

In the formula (4), X^{a}, X^{b}, X^{c}, and X^{d} each independently represent a hydrogen atom, a fluorine atom, or a trifluoromethyl group, provided that at least one of X^{a}, X^{b}, X^{c}, and X^{d} is a fluorine atom or a trifluoromethyl group.

In the formula (4), “k” represents 0, 1, 2, or 3.

As the photo-acid generator represented by the formula (4), one represented by the following formula (4′) is preferable.

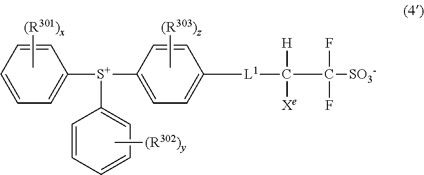

In the formula (4′), L¹ is as defined above. X^{e} represents a hydrogen atom or a trifluoromethyl group, preferably a trifluoromethyl group. R³⁰¹, R³⁰², and R³⁰³ each independently represent a hydrocarbyl group having 1 to 20 carbon atoms and optionally containing a heteroatom. The hydrocarbyl group may be saturated or unsaturated, and may be linear, branched, or cyclic. Specific examples thereof include those given as examples of the hydrocarbyl group represented by R^{fa1} in the formula (3A′). “x” and “y” each independently represent 0, 1, 2, 3, 4, or 5. “z” represents 0, 1, 2, 3, or 4.

Specific examples of the photo-acid generator represented by the formula (4) include those given as examples of a photo-acid generator represented by a formula (2) in JP 2017-26980 A.

Among the above-described photo-acid generators, those containing the anion represented by the formula (3A′) or (3D) are particularly preferable because of small acid diffusion and excellent solubility to a solvent. A photo-acid generator represented by the formula (4′) is also particularly preferable because the acid diffusion is very small.

Furthermore, as other acid generators, it is also possible to use a sulfonium salt or iodonium salt containing an anion having an aromatic ring substituted with an iodine atom represented by the following formula (5-1) or (5-2).

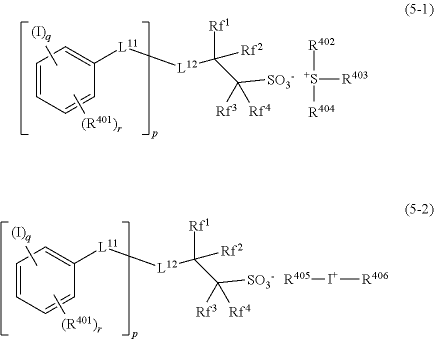

In the formulae (5-1) and (5-2), “p” represents 1, 2, or 3. “q” represents 1, 2, 3, 4, or 5. “r” represents 0, 1, 2, or 3. Here, 1≤q+r≤5. “q” is preferably 1, 2, or 3, more preferably 2 or 3. “r” is preferably 0, 1, or 2.

In the formulae (5-1) and (5-2), L¹¹ represents a single bond, an ether bond, an ester bond, or a saturated hydrocarbylene group having 1 to 6 carbon atoms and optionally including an ether bond or an ester bond. The saturated hydrocarbylene group may be linear, branched, or cyclic.

In the formulae (5-1) and (5-2), L¹² represents a single bond or a divalent linking group having 1 to 20 carbon atoms when “p” is 1, and represents a (p+1)-valent linking group having 1 to 20 carbon atoms when “p” is 2 or 3, the linking group optionally containing an oxygen atom, a sulfur atom, or a nitrogen atom.

In the formulae (5-1) and (5-2), R⁴⁰1 represents a hydroxy group, a carboxy group, a fluorine atom, a chlorine atom, a bromine atom, an amino group, a hydrocarbyl group having 1 to 20 carbon atoms, a hydrocarbyloxy group having 1 to 20 carbon atoms, a hydrocarbylcarbonyl group having 2 to 20 carbon atoms, a hydrocarbyloxycarbonyl group having 2 to 20 carbon atoms, a hydrocarbylcarbonyloxy group having 2 to 20 carbon atoms, a hydrocarbylsulfonyloxy group having 1 to 20 carbon atoms, —N(R^{401A})(R^{401B}), —N(R^{401C})—C(═O)—R^{401D}, or —N(R^{401C})—C(═O)—O—R^{401D}. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group optionally contain a fluorine atom, a chlorine atom, a bromine atom, a hydroxy group, an amino group, or an ether bond. R^{401A} and R^{401B} each independently represent a hydrogen atom or a saturated hydrocarbyl group having 1 to 6 carbon atoms. R^{401C} represents a hydrogen atom or a saturated hydrocarbyl group having 1 to 6 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. R^{401D} represents an aliphatic hydrocarbyl group having 1 to 16 carbon atoms, an aryl group having 6 to 14 carbon atoms, or an aralkyl group having 7 to 15 carbon atoms, and optionally contains a halogen atom, a hydroxy group, a saturated hydrocarbyloxy group having 1 to 6 carbon atoms, a saturated hydrocarbylcarbonyl group having 2 to 6 carbon atoms, or a saturated hydrocarbylcarbonyloxy group having 2 to 6 carbon atoms. The aliphatic hydrocarbyl group may be saturated or unsaturated, and may be linear, branched, or cyclic. The hydrocarbyl group, hydrocarbyloxy group, hydrocarbylcarbonyl group, hydrocarbyloxycarbonyl group, hydrocarbylcarbonyloxy group, and hydrocarbylsulfonyloxy group may be linear, branched, or cyclic. When “p” and/or “r” is 2 or more, the R⁴⁰¹s may be identical to or different from one another.

Among these, a hydroxy group, —N(R^{401C})—C(═O)—R^{401D}, —N(R^{401C})—C(═O)—O—R^{401D}, a fluorine atom, a chlorine atom, a bromine atom, a methyl group, a methoxy group, etc. are preferable as R⁴⁰.

In the formulae (5-1) and (5-2), Rf¹ to Rf⁴ each independently represent a hydrogen atom, a fluorine atom, or a trifluoromethyl group, provided that at least one of Rf¹ to Rf⁴ is a fluorine atom or a trifluoromethyl group. Rf¹ and Rf² may also be combined to form a carbonyl group. In particular, Rf³ and Rf⁴ are preferably both a fluorine atom.

In the formulae (5-1) and (5-2), R⁴⁰² to R⁴⁰⁶ each independently represent a halogen atom or a hydrocarbyl group having 1 to 20 carbon atoms and optionally containing a heteroatom. The hydrocarbyl group may be saturated or unsaturated, and may be linear, branched, or cyclic. Specific examples thereof include those given as examples of the hydrocarbyl groups represented by R^{ct1} to R^{ct5} in the description of the formulae (Z-1) and (Z-2). Furthermore, part or all of the hydrogen atoms of the hydrocarbyl group may be substituted with a hydroxy group, a carboxy group, a halogen atom, a cyano group, a nitro group, a mercapto group, a sultone ring, a sulfo group, or a sulfonium-salt-containing group, and part of the —CH₂— of the hydrocarbyl group may be substituted with an ether bond, an ester bond, a carbonyl group, an amide bond, a carbonate bond, or a sulfonic acid ester bond. Furthermore, R⁴⁰² and R⁴⁰³ may be bonded to each other to form a ring together with the sulfur atom bonded thereto. In this event, specific examples of the ring include those given as examples of the rings that can be formed by R^{ct1} and R^{ct2} being bonded to each other together with the sulfur atom bonded thereto in the description of the formula (Z-1).

Specific examples of the cation of the sulfonium salt represented by the formula (5-1) include those given as examples of the sulfonium cation represented by the formula (Z-1). Meanwhile, specific examples of the cation of the iodonium salt represented by the formula (5-2) include those given as examples of the iodonium cation represented by the formula (Z-2).

Specific examples of the anion of the onium salt represented by the formula (5-1) or (5-2) include the following, but are not limited thereto.

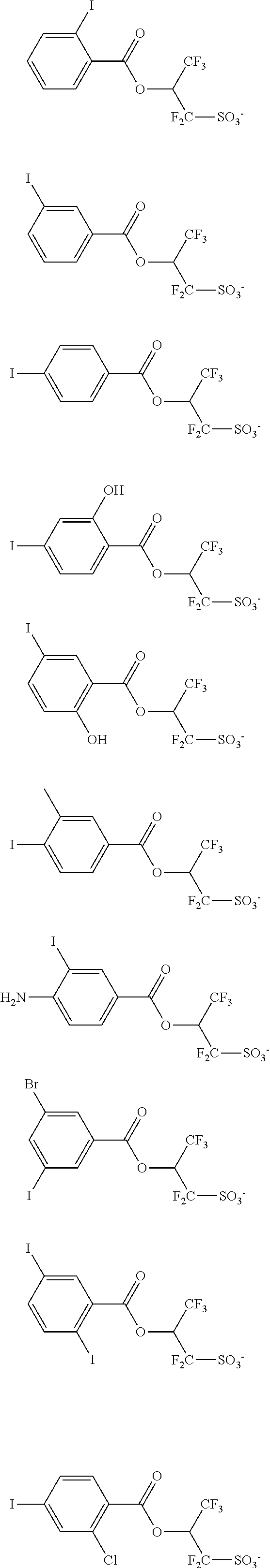

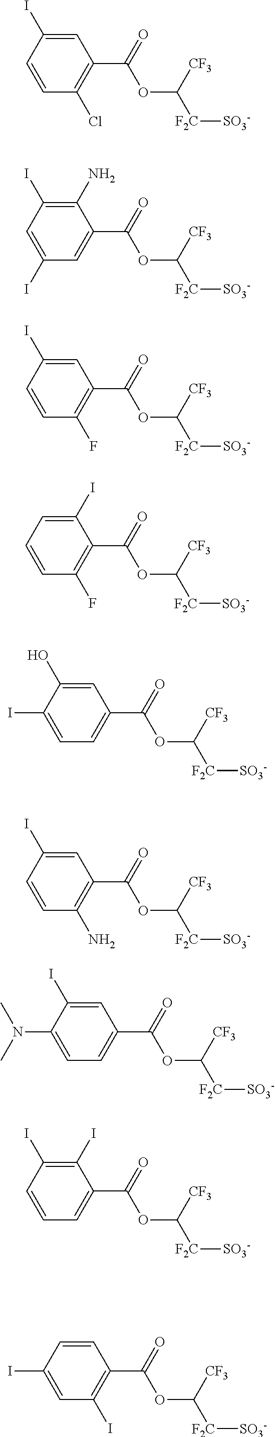

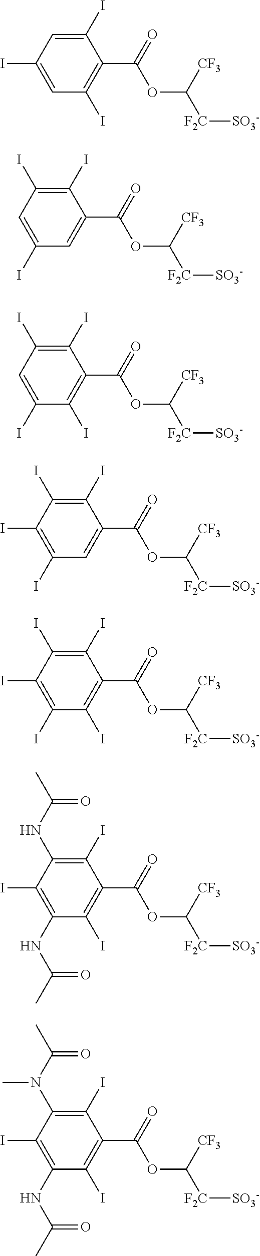

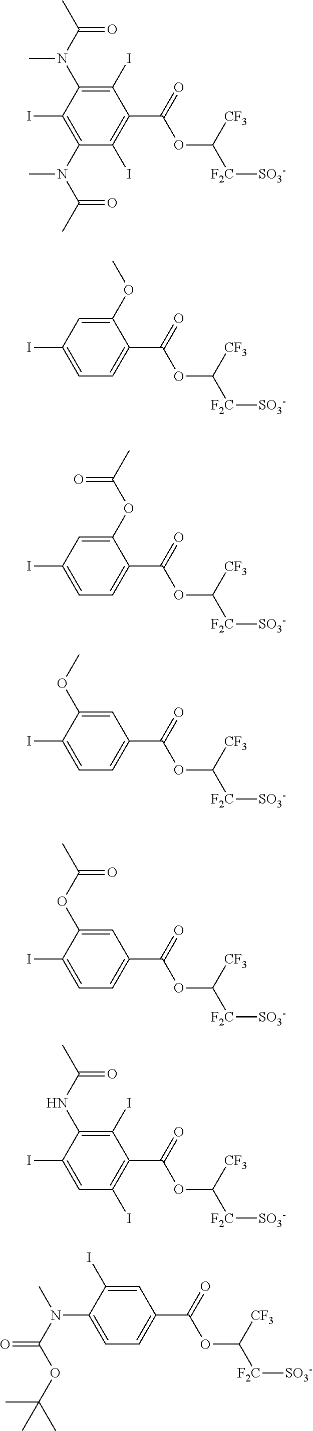

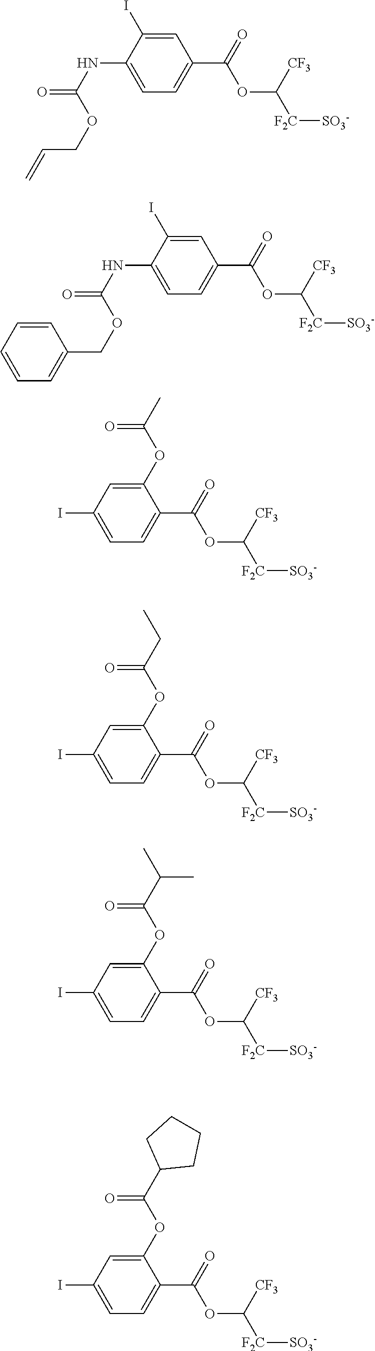

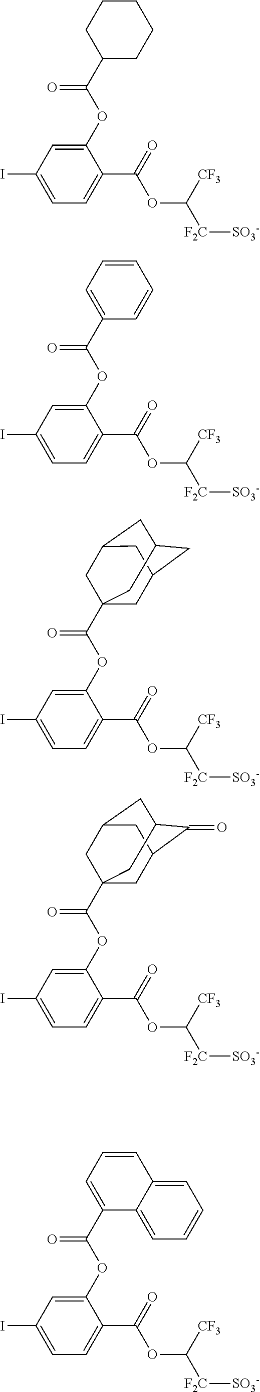

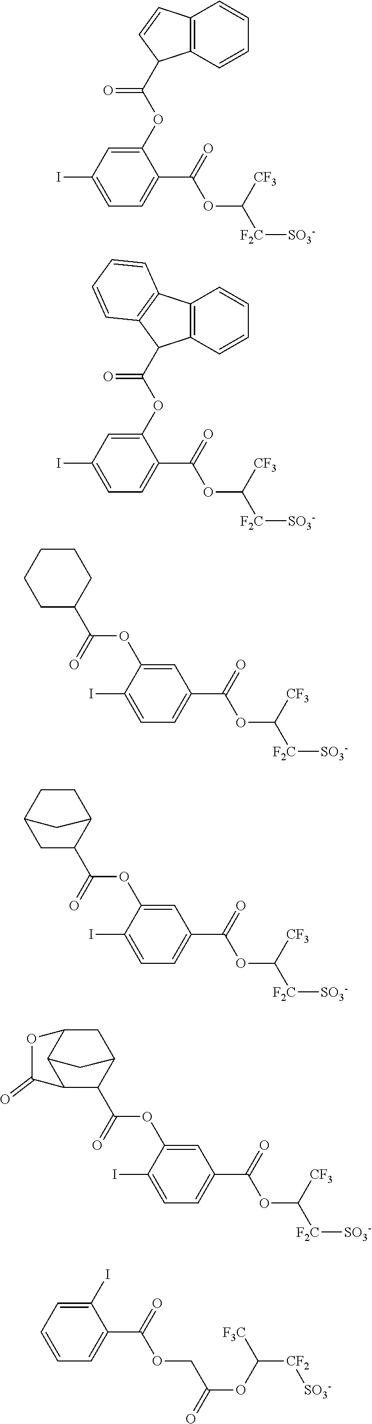

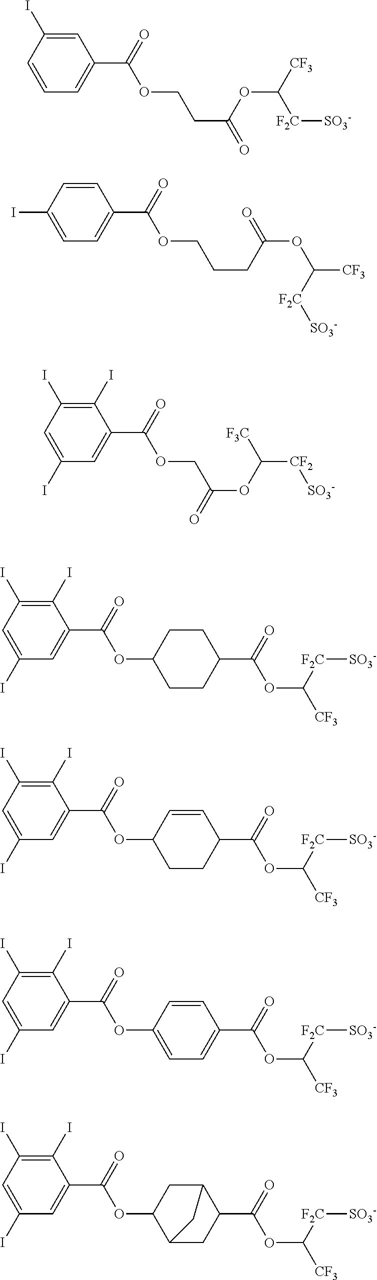

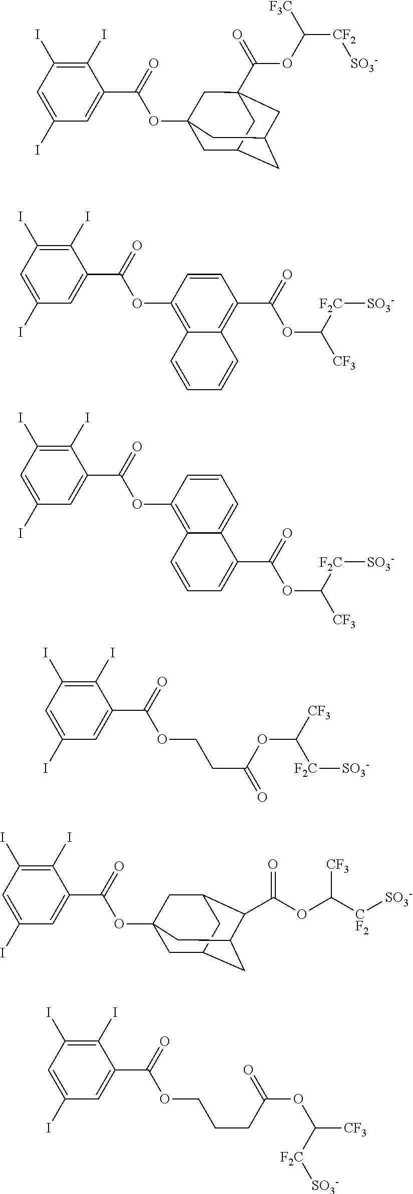

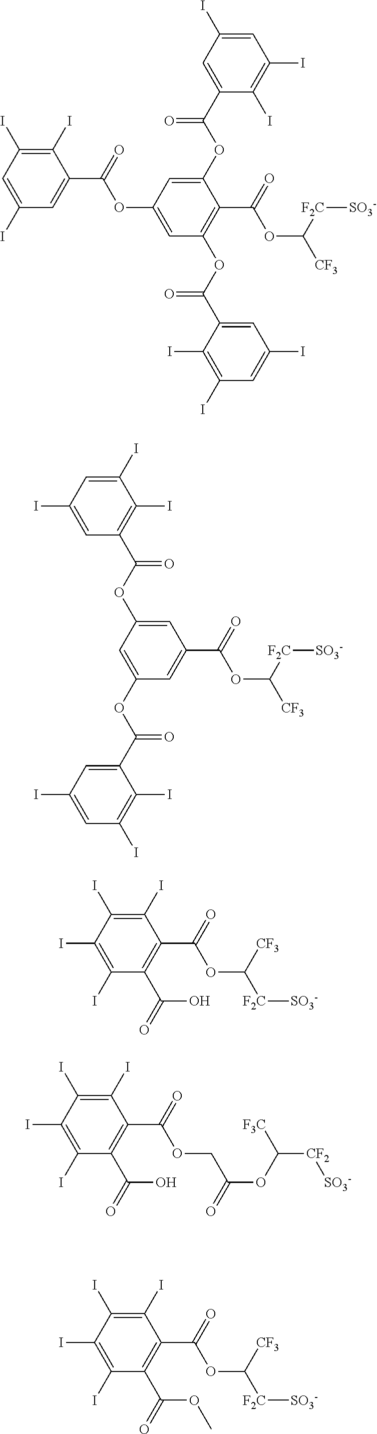

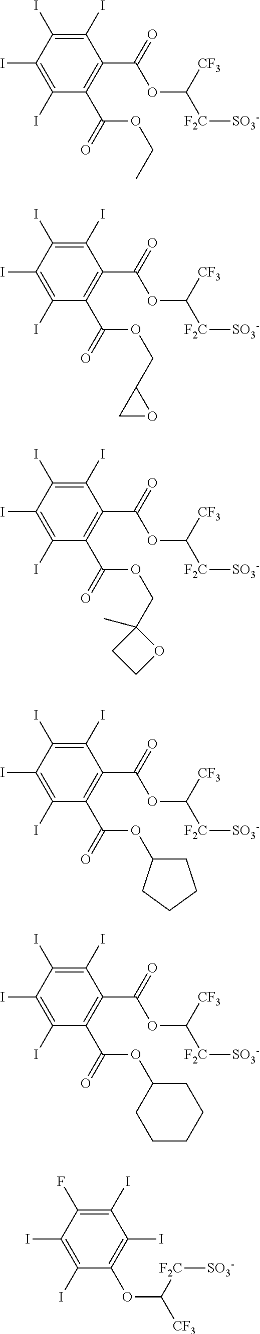

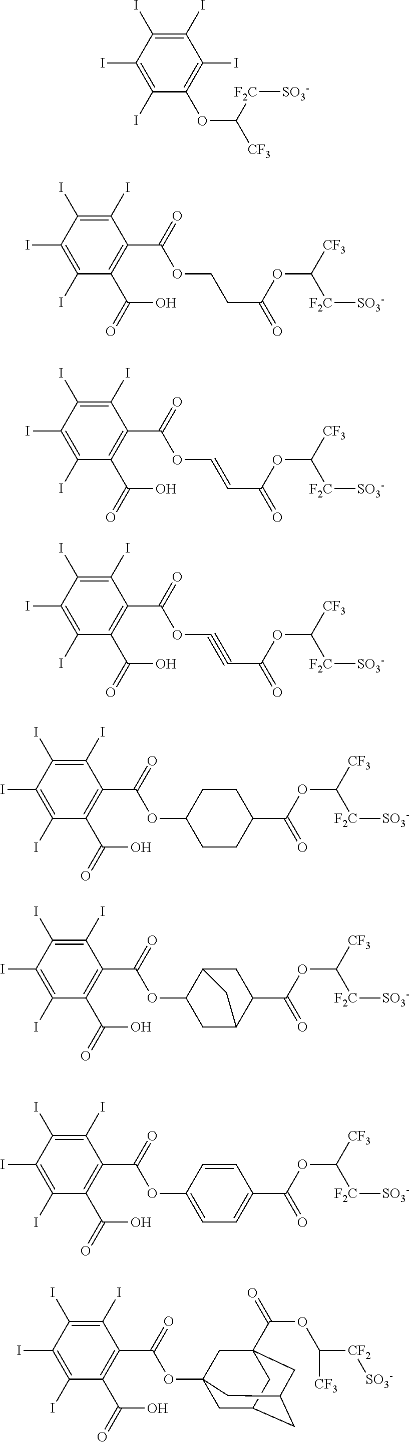

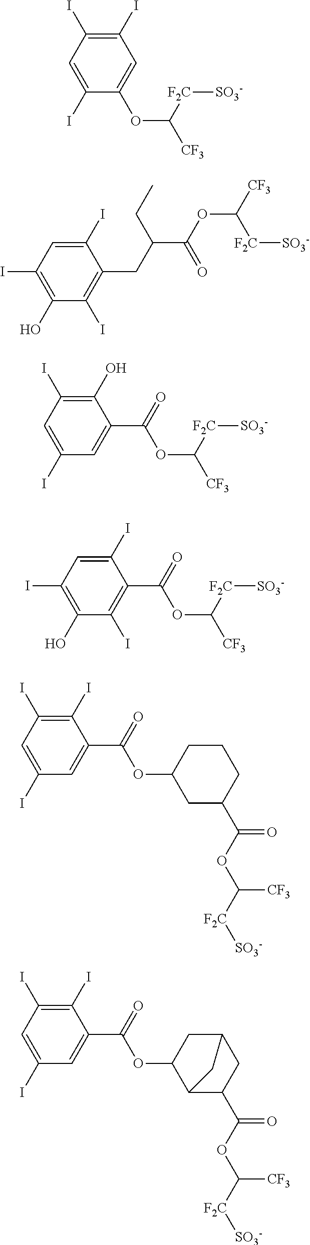

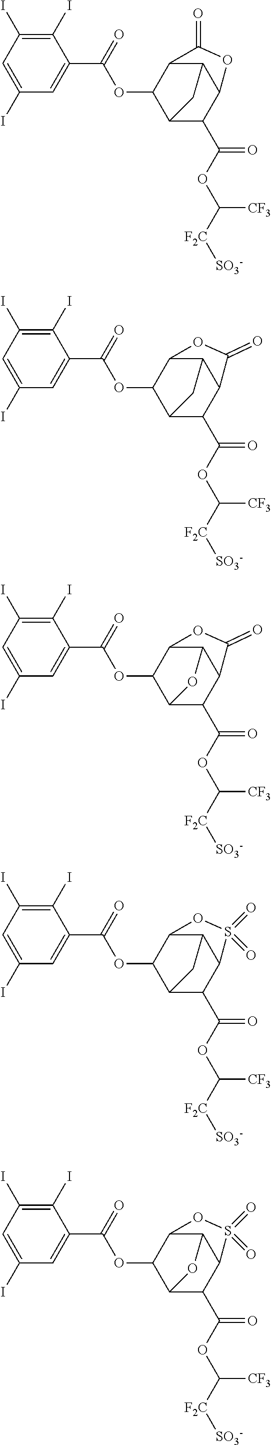

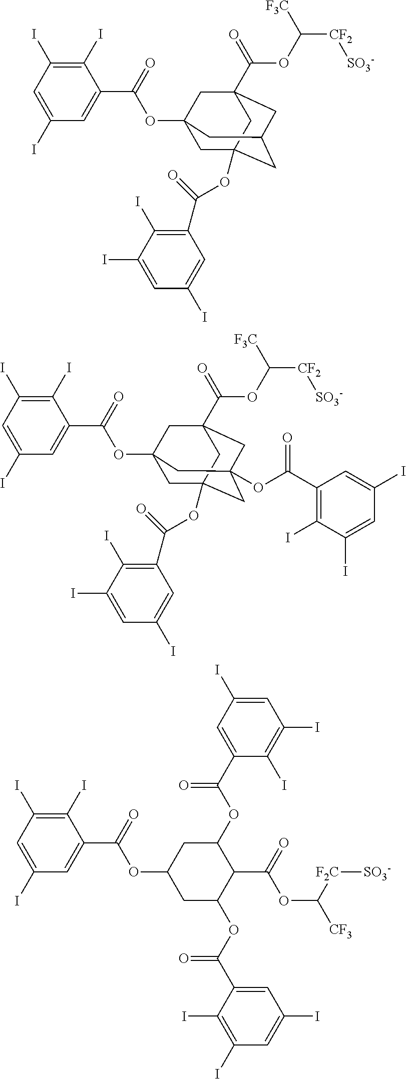

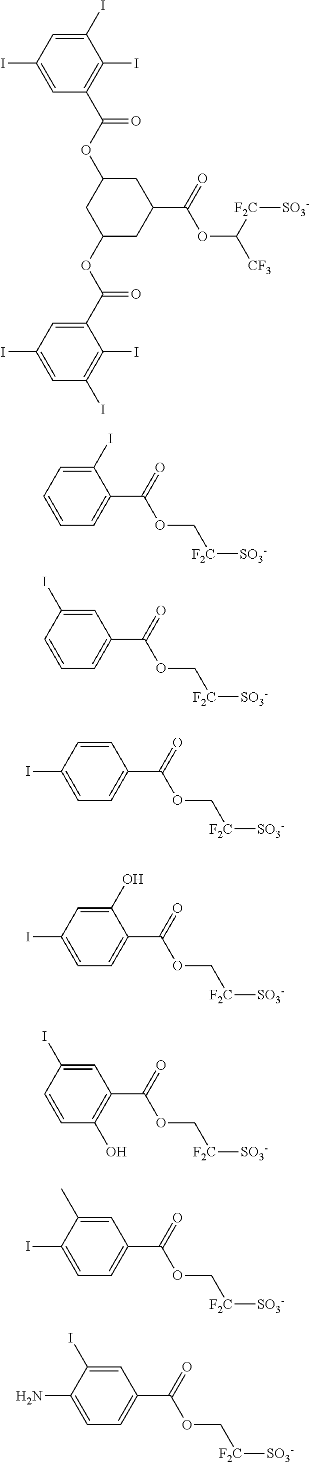

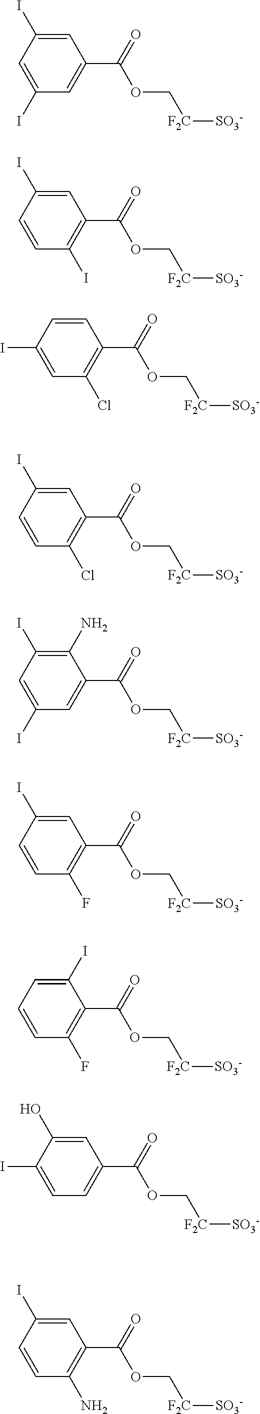

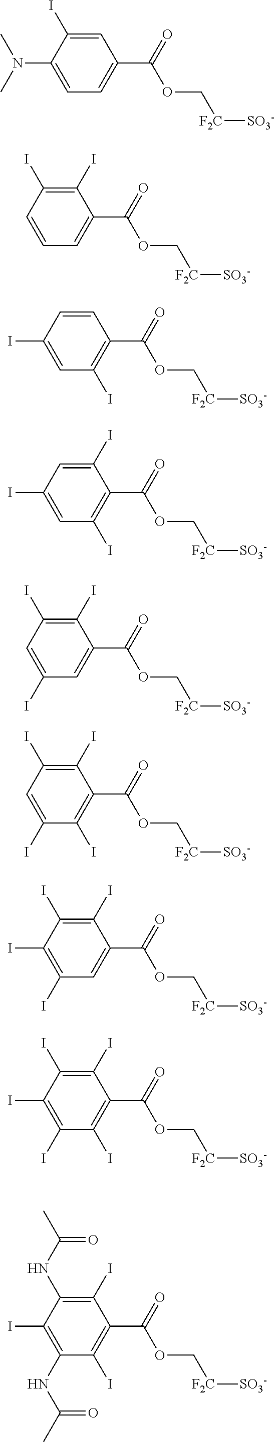

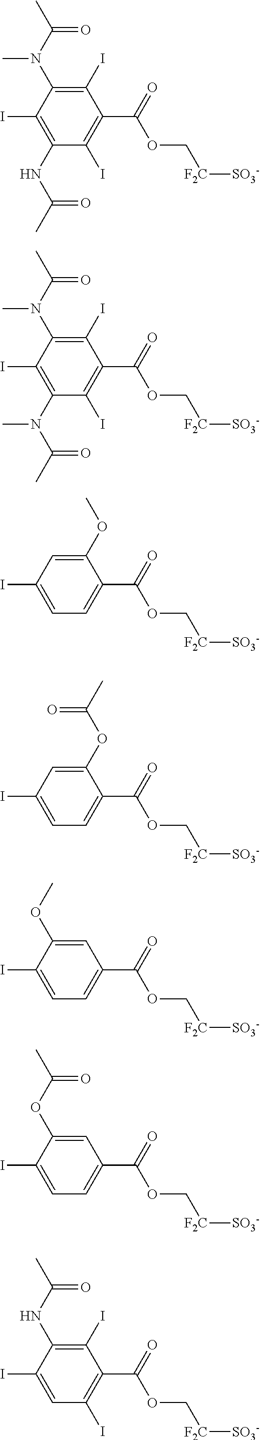

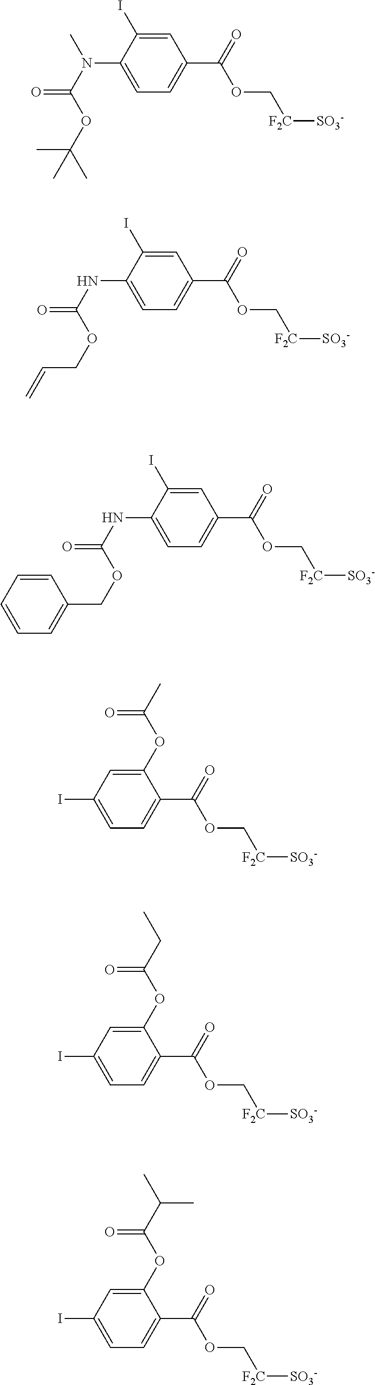

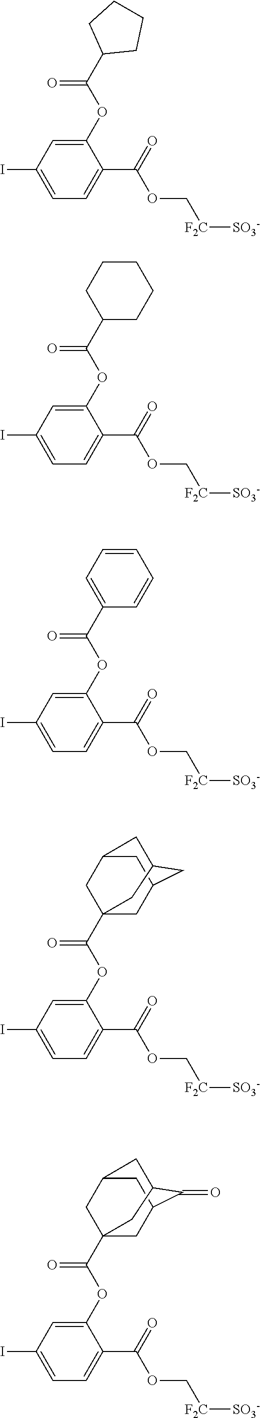

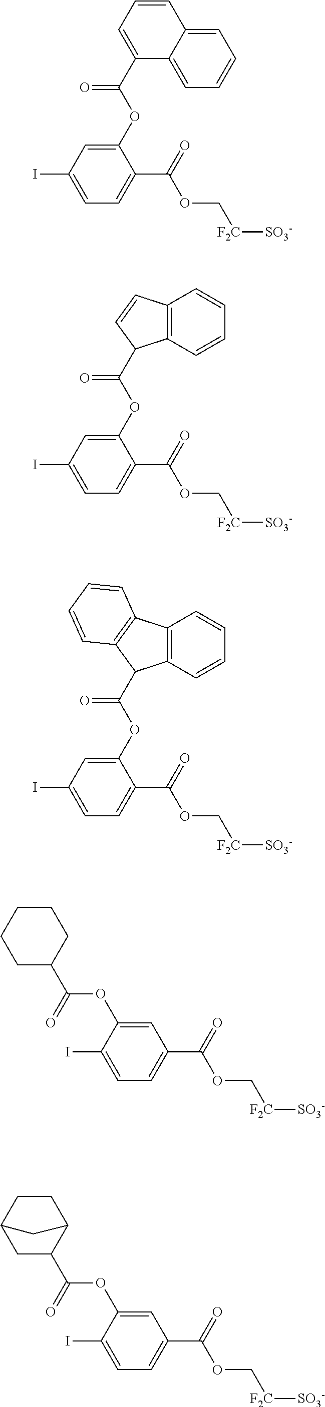

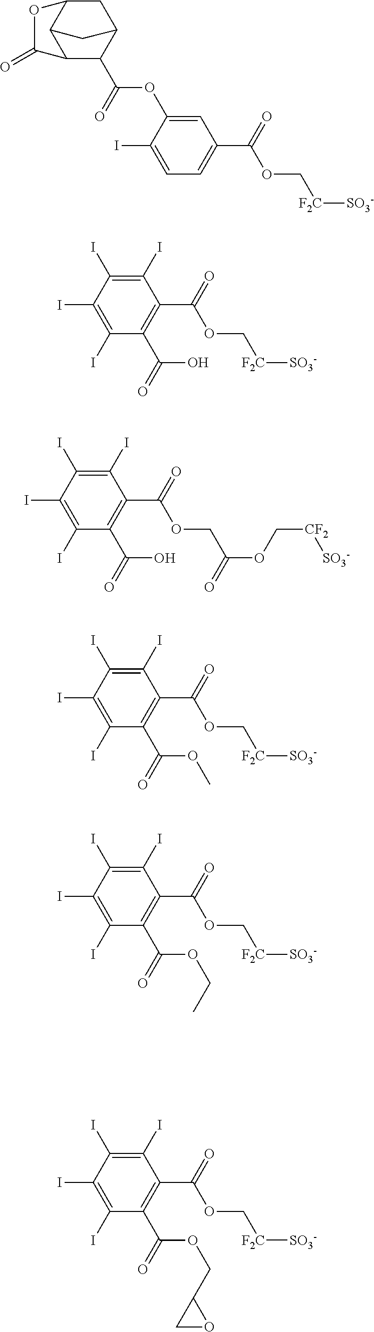

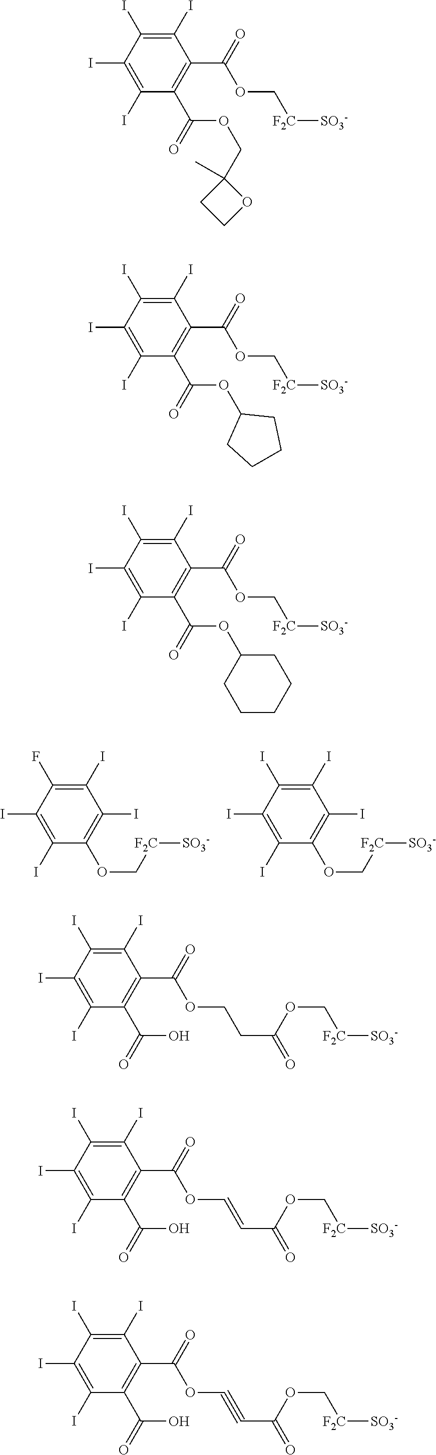

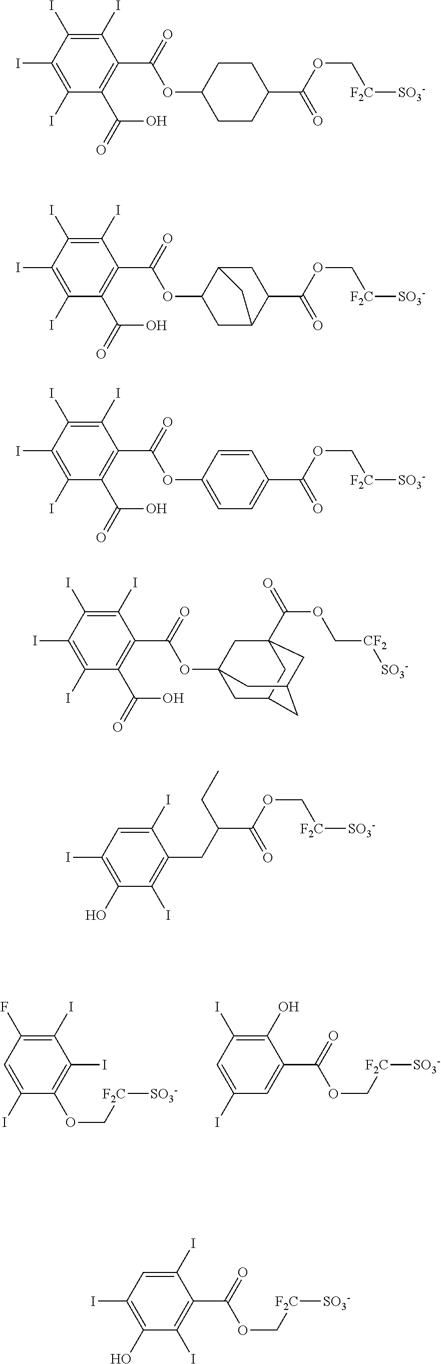

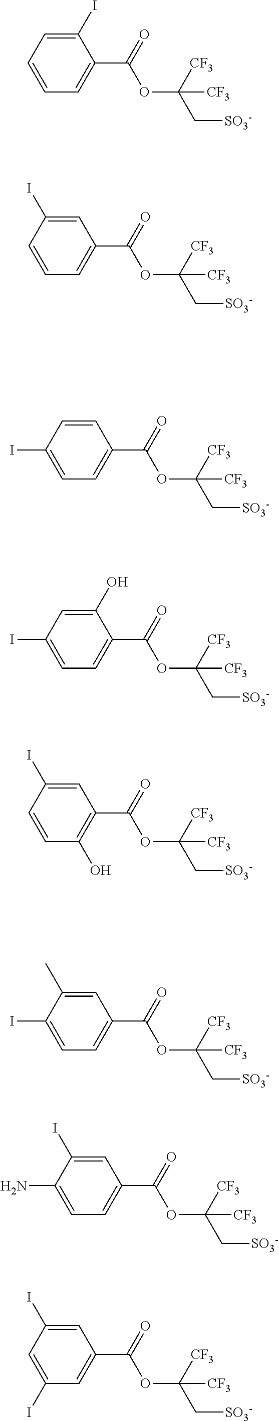

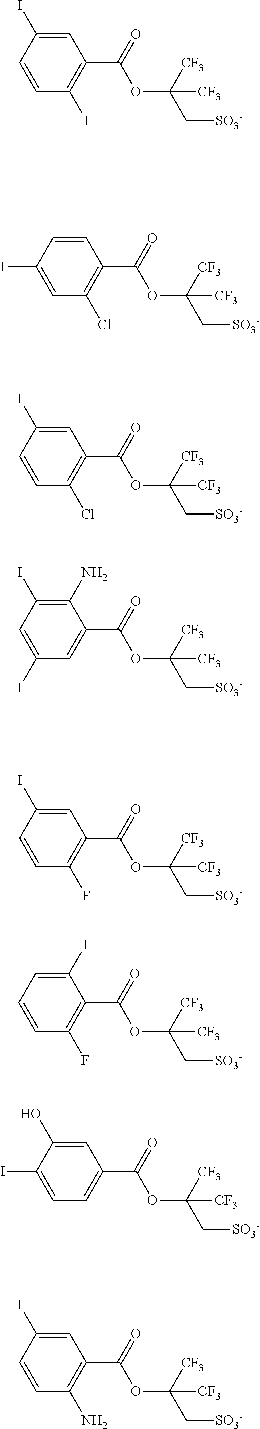

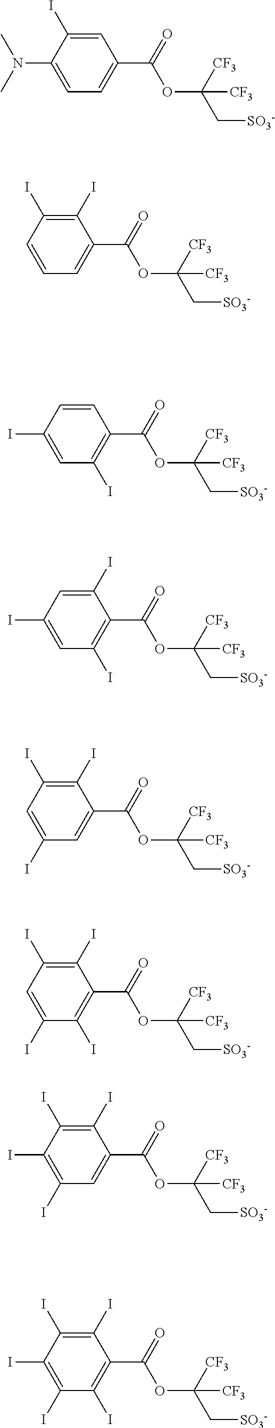

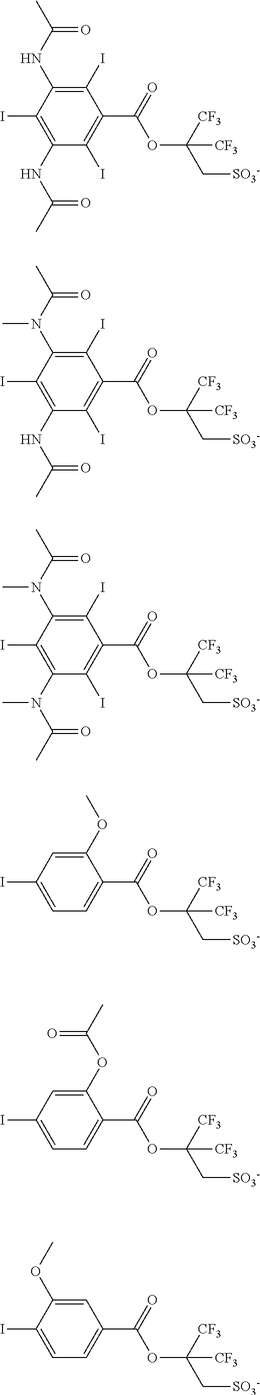

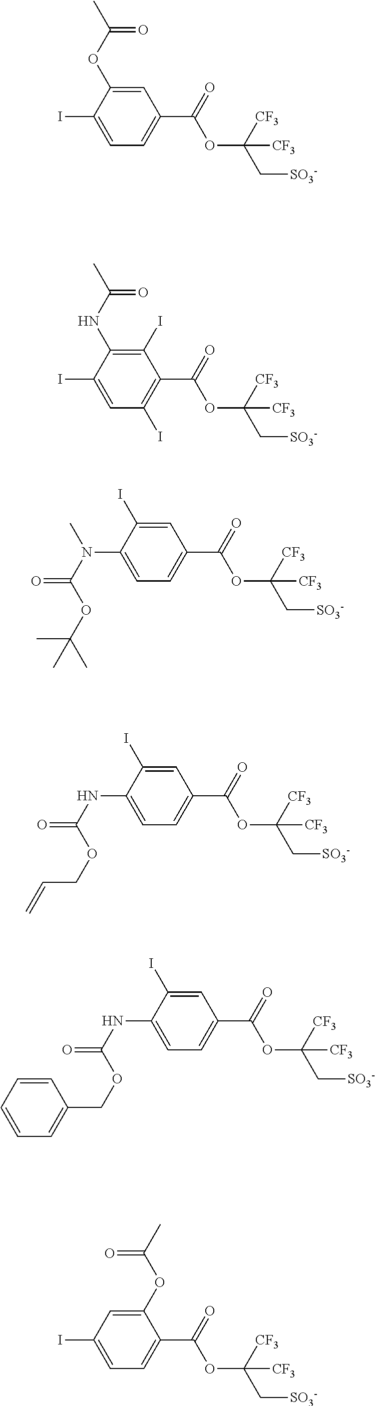

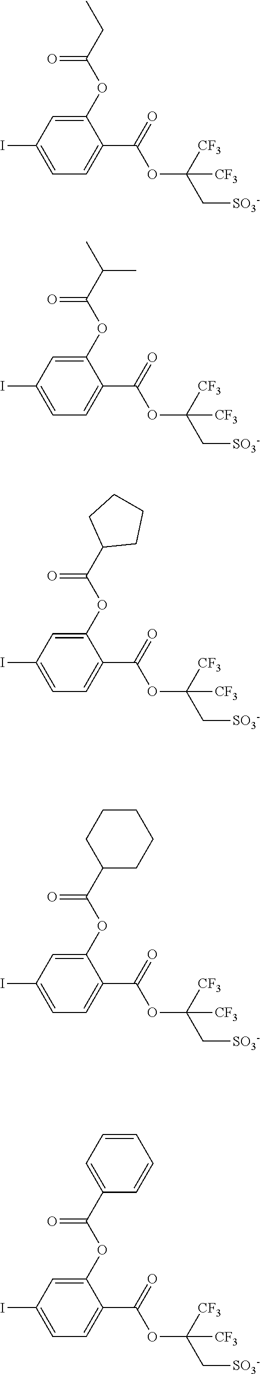

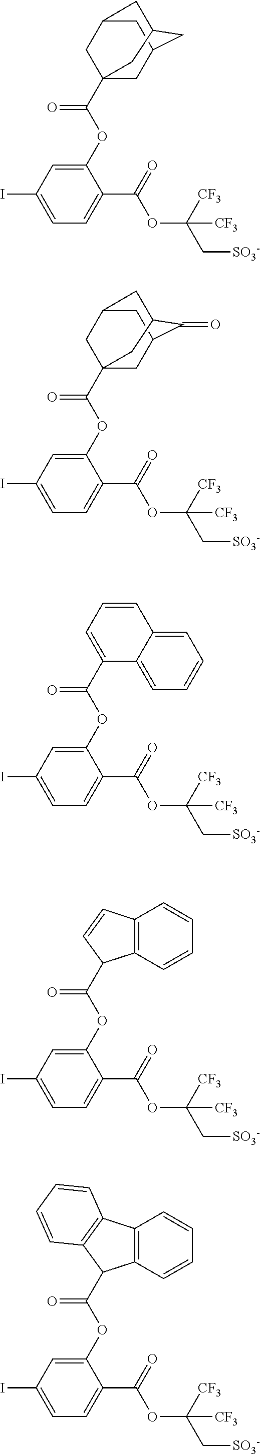

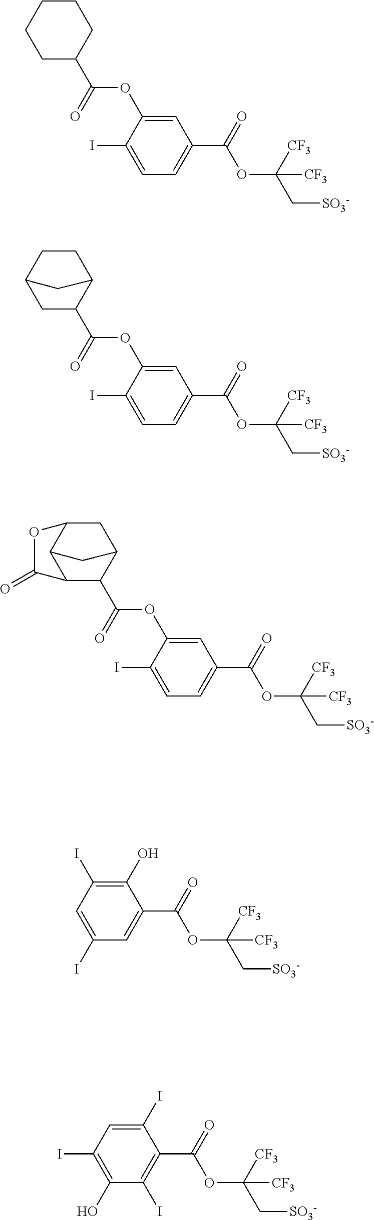

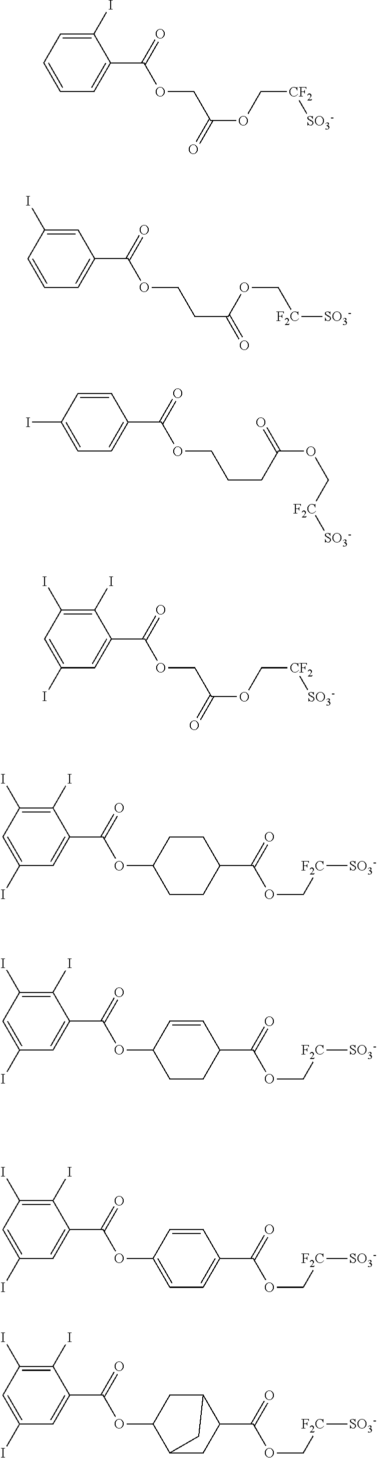

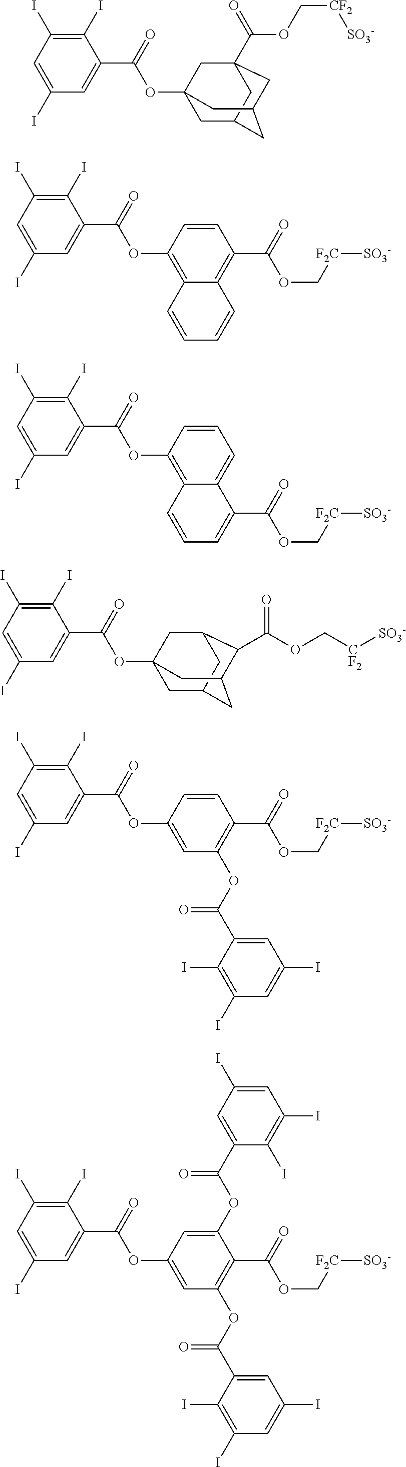

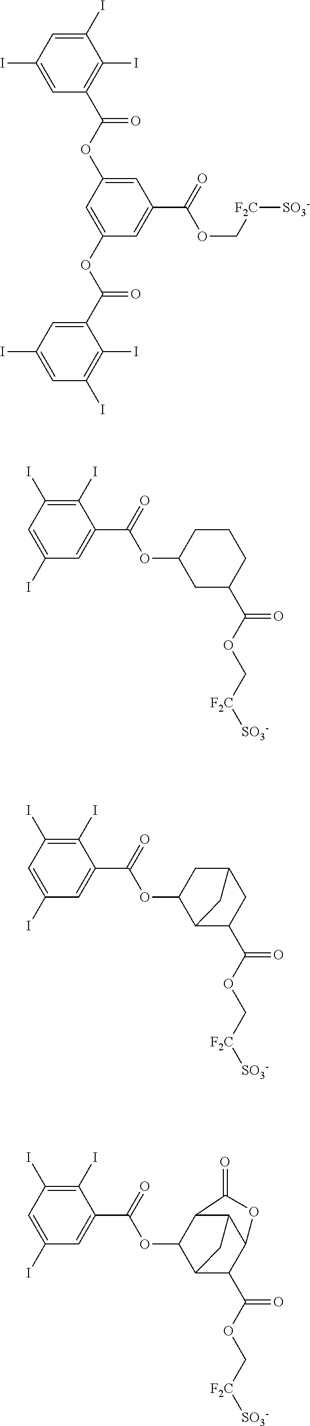

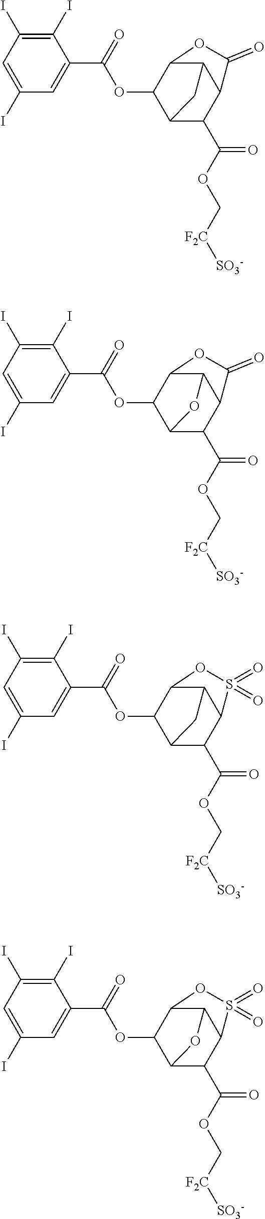

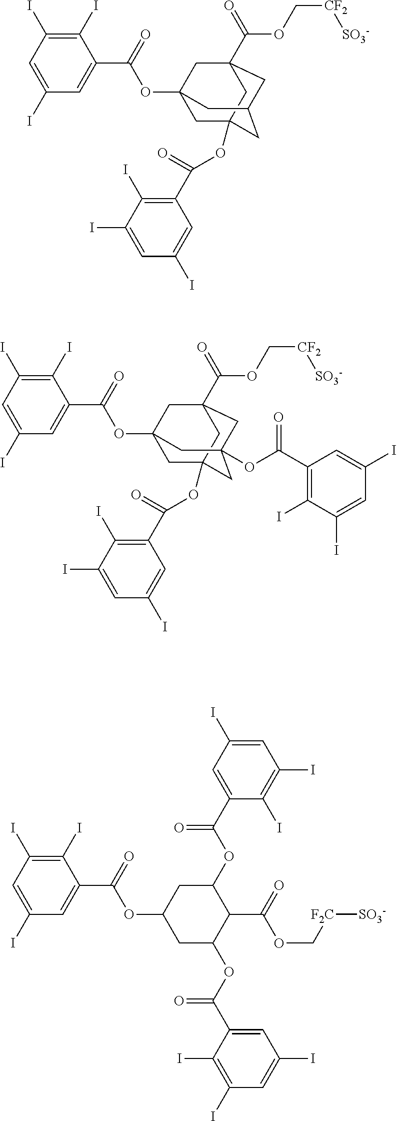

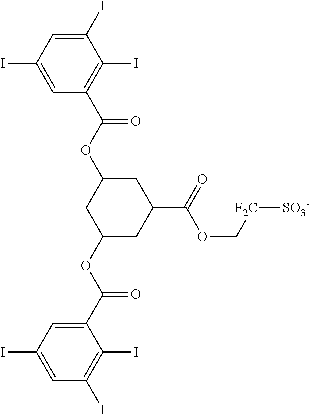

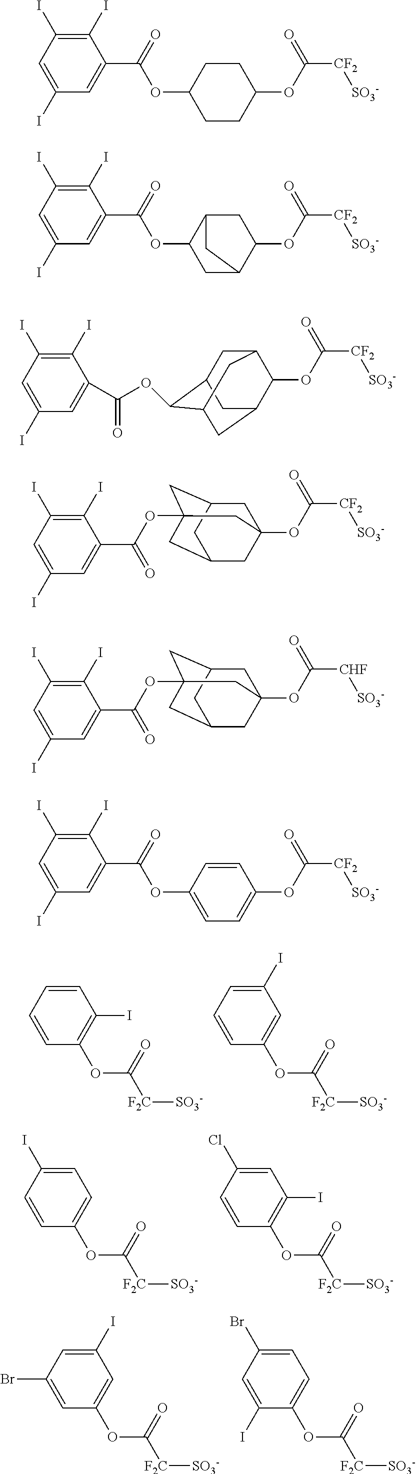

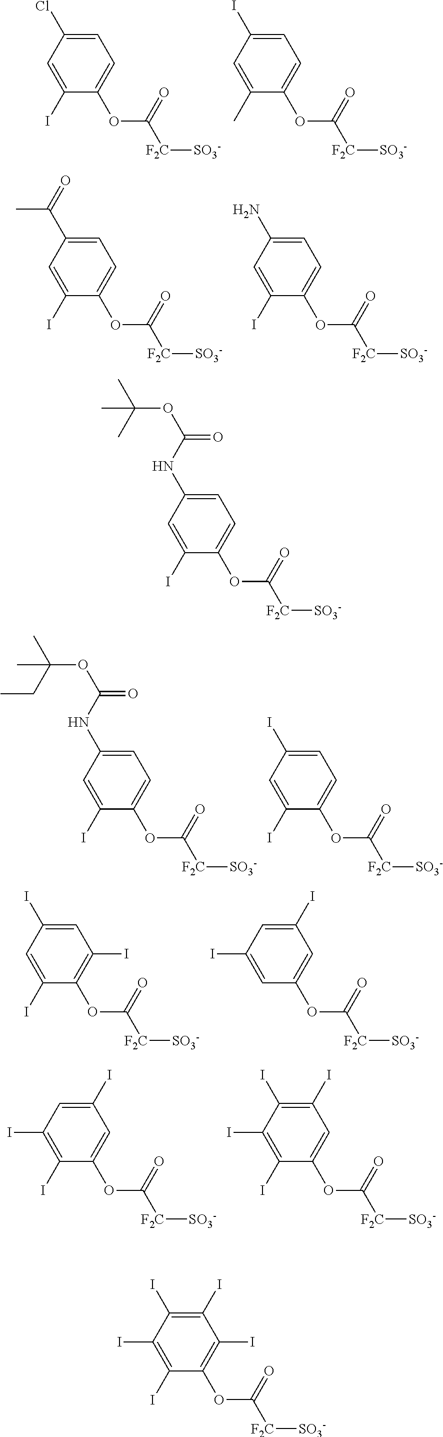

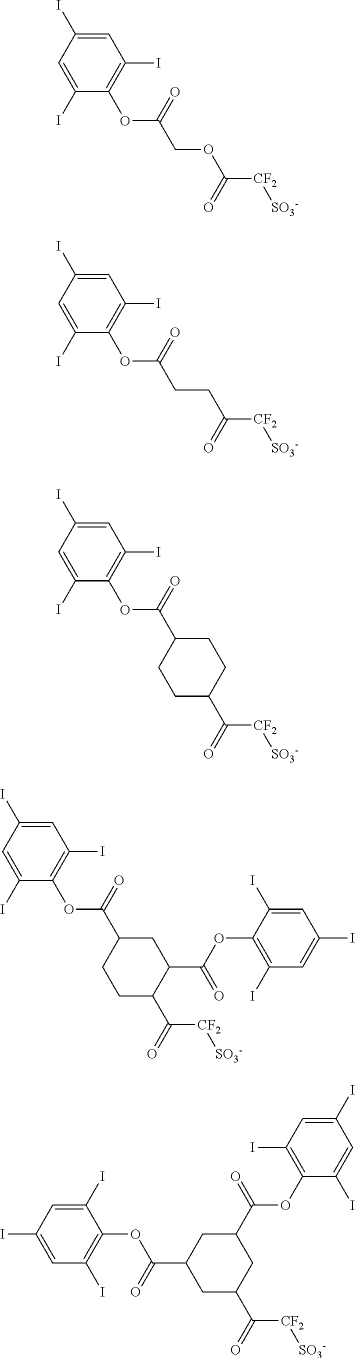

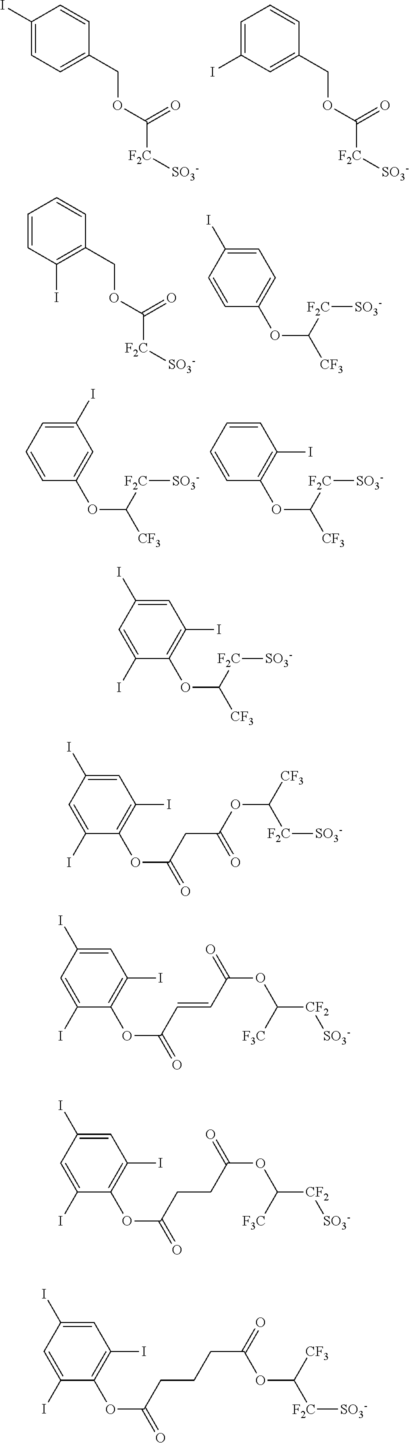

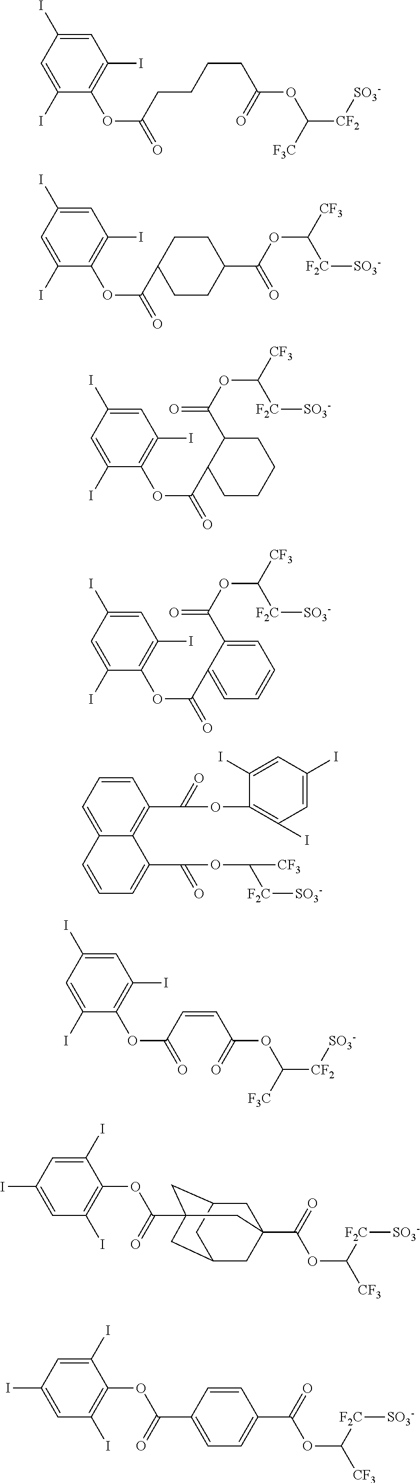

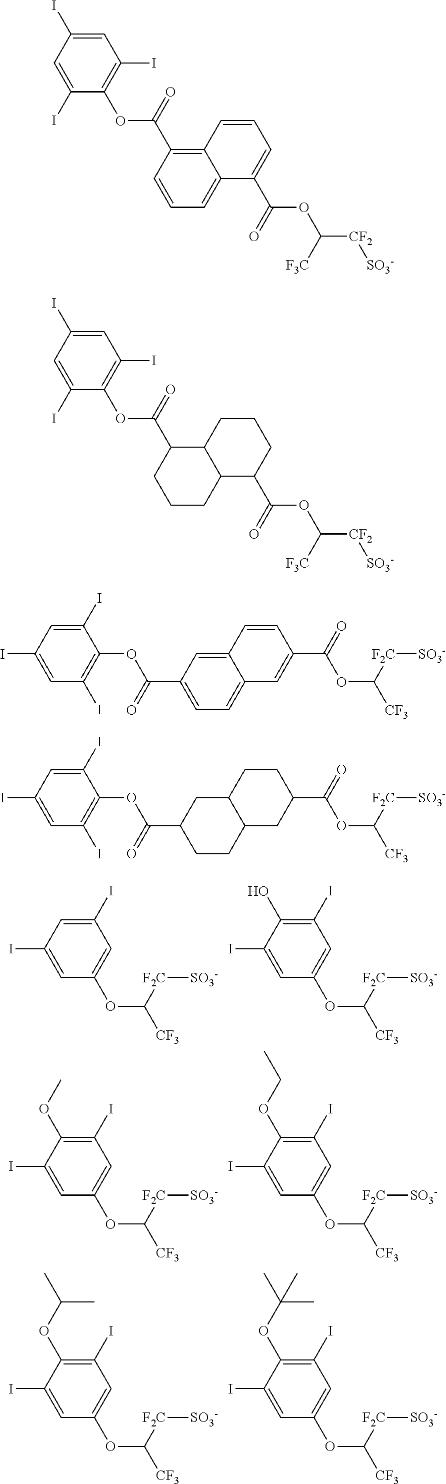

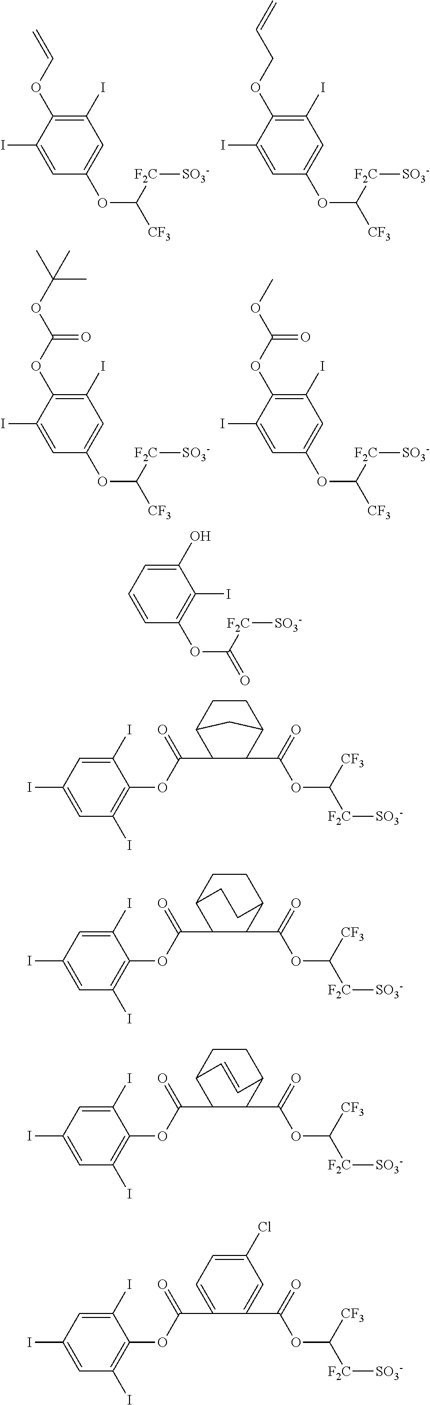

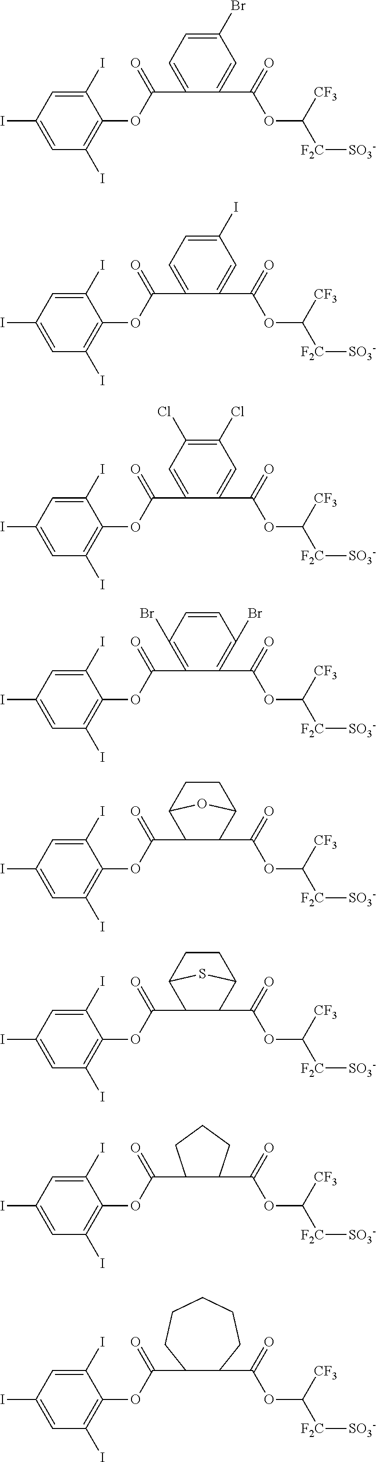

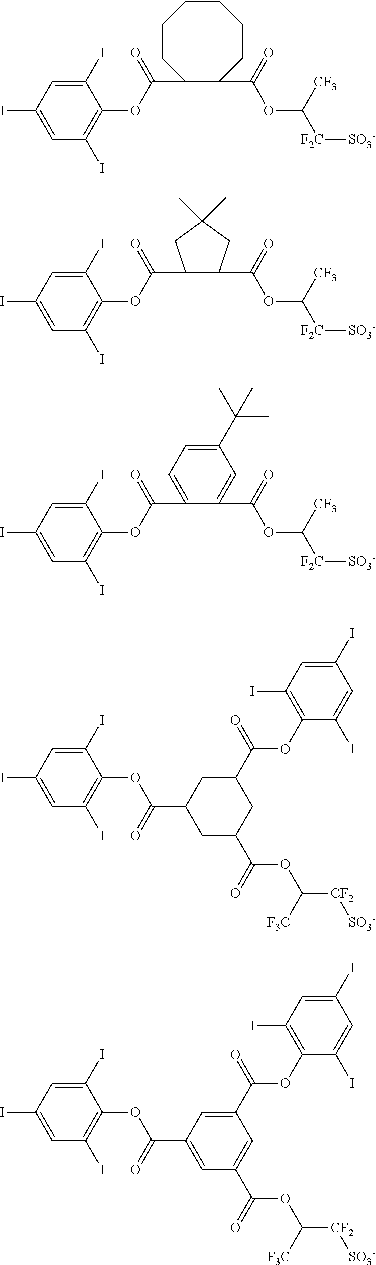

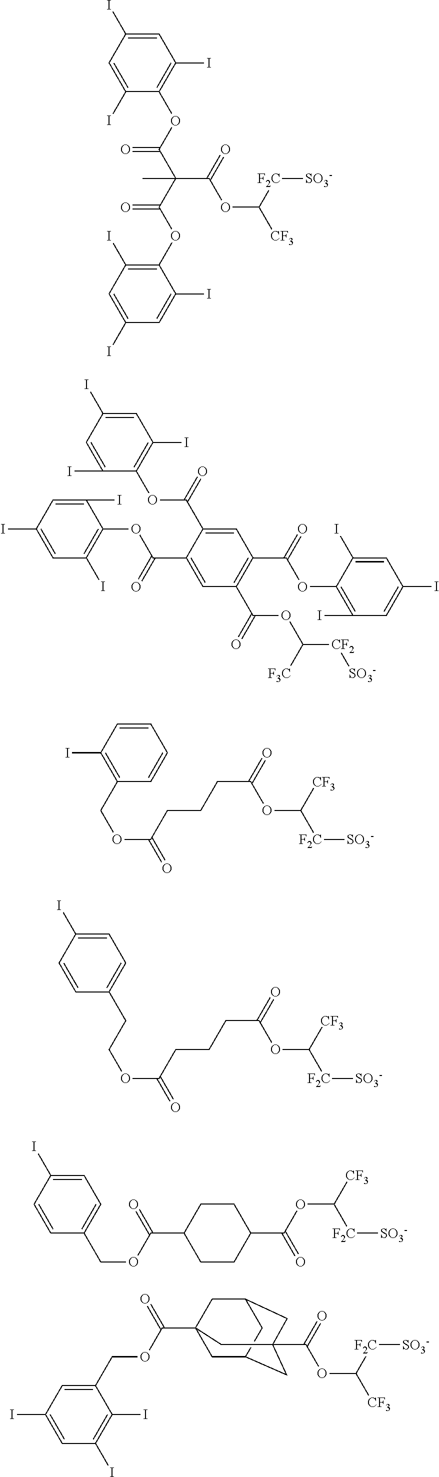

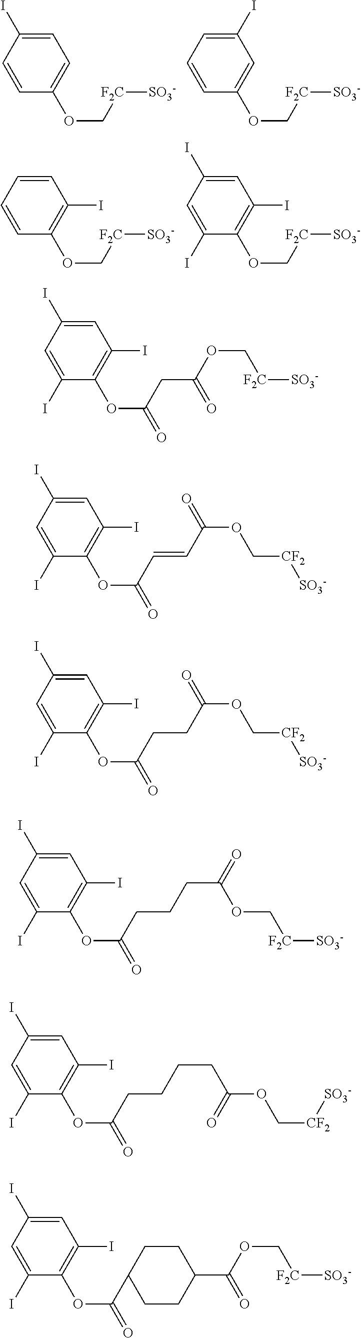

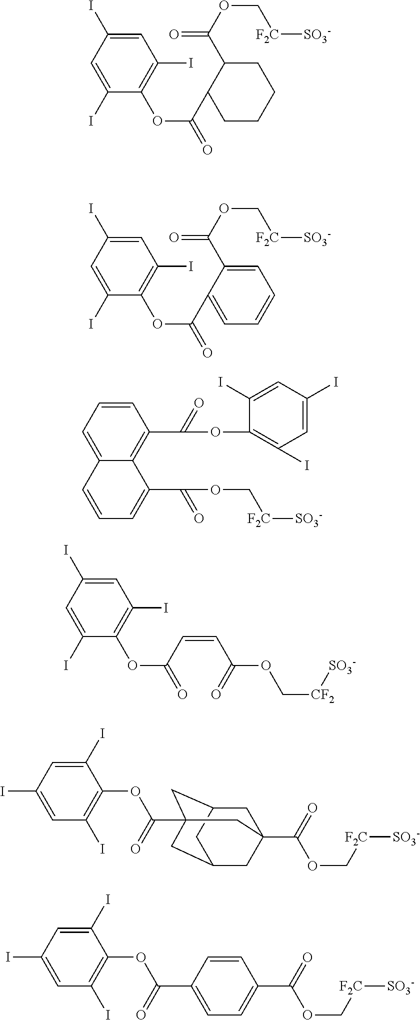

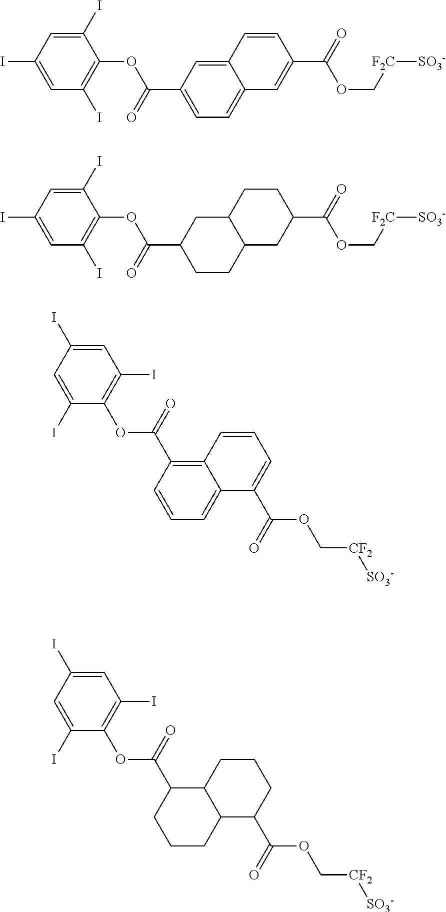

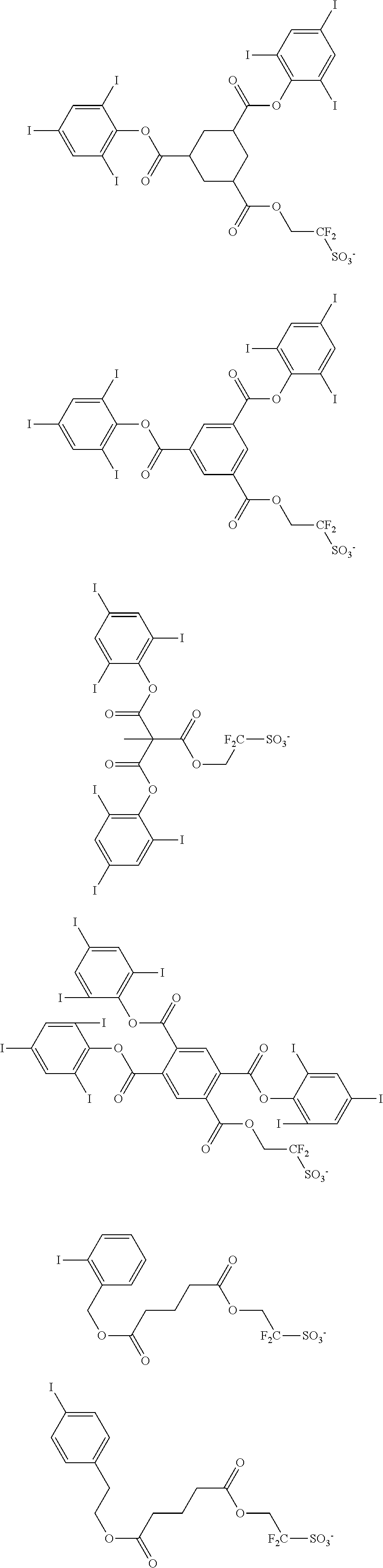

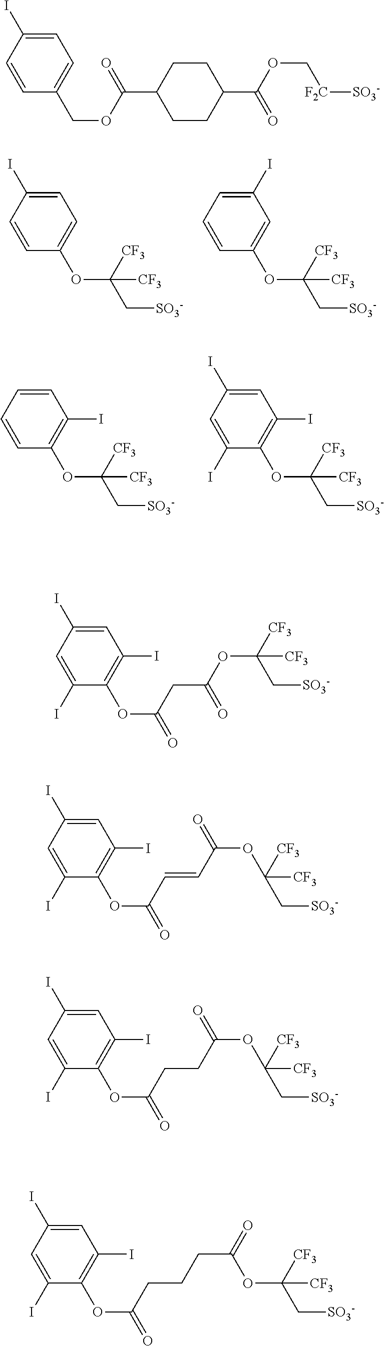

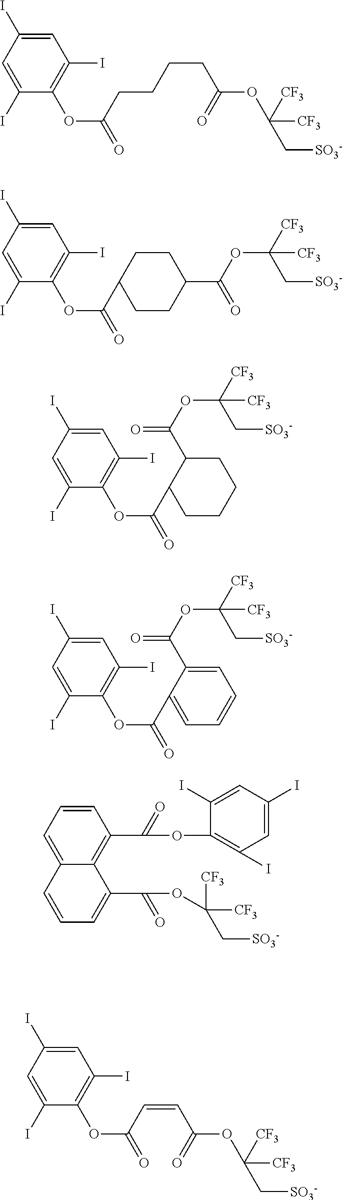

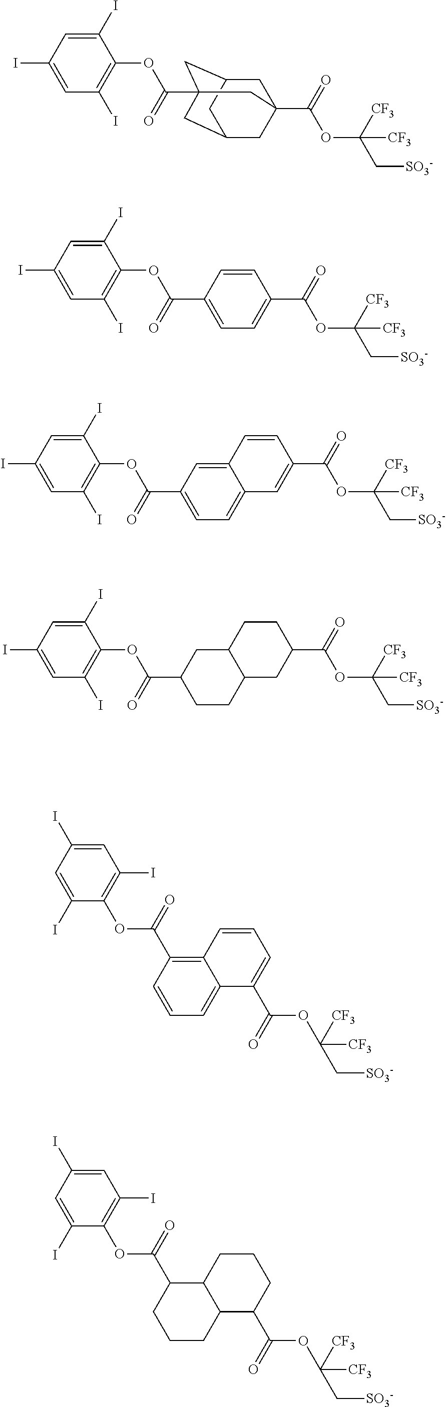

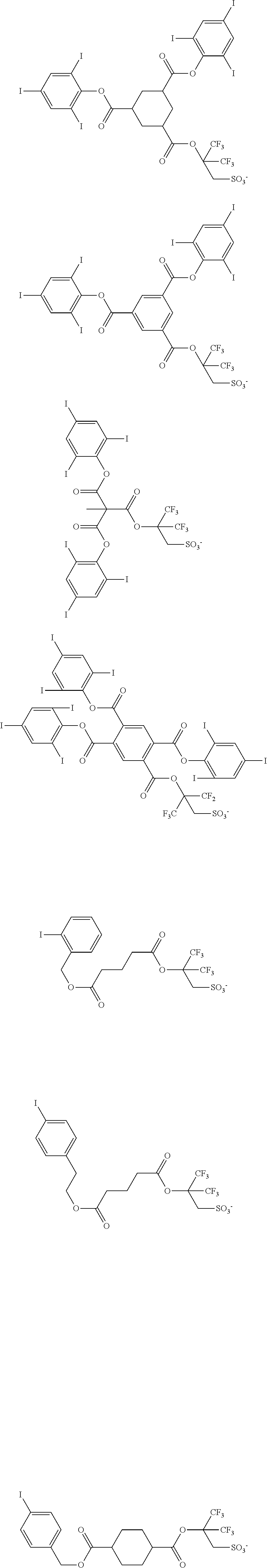

When the inventive chemically-amplified resist composition contains an acid generator (D), the contained amount is preferably 0.1 to 40 parts by mass, more preferably 0.5 to 20 parts by mass based on 80 parts by mass of the base polymer (A). When the contained amount of the component (D) acid generator is in the above-described ranges, resolution is favorable, and there is no risk of a problem of foreign substances occurring after the development of or when delaminating the resist film. Therefore, such ranges are preferable. One kind of the acid generator (D) may be used, or two or more kinds thereof may be used in combination.

### [(E) Surfactant]

The inventive chemically-amplified resist composition may further contain a surfactant as a component (E). The surfactant (E) is preferably a surfactant that is insoluble or hardly soluble in water and soluble in alkaline developers, or a surfactant that is insoluble or hardly soluble in water and alkaline developers. As such a surfactant, those disclosed in JP 2010-215608 A and JP 2011-16746 A can be referred to.

As the surfactant insoluble or hardly soluble in water and alkaline developers, among the surfactants disclosed in the above-mentioned publications, preferable are FC-4430 (manufactured by 3M Company), Surflon (registered trademark)S-381 (manufactured by AGC Seimi Chemical Co., Ltd.), OLFINE (registered trademark) E1004 (manufactured by Nissin Chemical Industry Co., Ltd.), KH-20 and KH-30 (manufactured by AGC Seimi Chemical Co., Ltd.), an oxetane ring-opened polymer represented by the following formula (surf-1), etc.

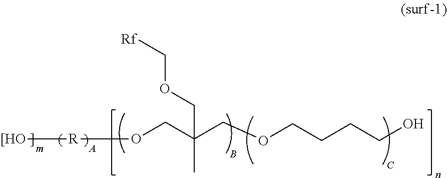

Here, R, Rf, A, B, C, “m”, and “n” apply only to the formula (surf-1), regardless of the definitions given above. R represents an aliphatic group having a valency of 2 to 4 and having 2 to 5 carbon atoms. As the aliphatic group, examples of divalent groups include an ethylene group, a 1,4-butylene group, a 1,2-propylene group, a 2,2-dimethyl-1,3-propylene group, and a 1,5-pentylene group, and examples of trivalent or tetravalent groups include the following.

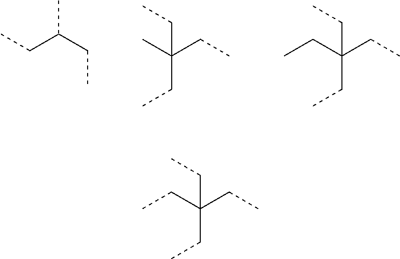

In the formulae, a broken line represents an attachment point, and each formula respectively represents a partial structure derived from glycerol, trimethylolethane, trimethylolpropane, and pentaerythritol.

Among these, a 1,4-butylene group, a 2,2-dimethyl-1,3-propylene group, etc. are preferable.

Rf represents a trifluoromethyl group or a pentafluoroethyl group, preferably a trifluoromethyl group. “m” represents an integer of 0 to 3, “n” represents an integer of 1 to 4, and the sum of “n” and “m” represents the valence of R and is an integer of 2 to 4. A represents 1. B represents an integer of 2 to 25, preferably an integer of 4 to 20. C represents an integer of 0 to 10, preferably 0 or 1. Furthermore, each constitutional unit in the formula (surf-1) does not define the arrangement, and the units may be bonded in blocks or at random. The production of the partially fluorinated oxetane ring-opened polymer-based surfactant is described in detail in U.S. Pat. No. 5,650,483 A etc.

A surfactant insoluble or hardly soluble in water and soluble in alkaline developers has a function of reducing penetration and leaching of water by being oriented on the resist film surface when a resist top coat is not used in ArF immersion lithography. Therefore, such a surfactant is useful for reducing damage to the exposure apparatus by suppressing elution of water-soluble components from the resist film, and is also useful since the surfactant is solubilized during development with an aqueous alkaline solution after exposure or after post-exposure baking (PEB) and hardly becomes a foreign substance that causes defects. Such a surfactant has a property that it is insoluble or hardly soluble in water and soluble in alkaline developers, and preferable is a polymer surfactant, in particular, one also called a hydrophobic resin and having a high water-repellency and capable of improving water-sliding property.

Specific examples of such a polymer surfactant include those including at least one kind selected from repeating units represented by the following formulae (6A) to (6E).

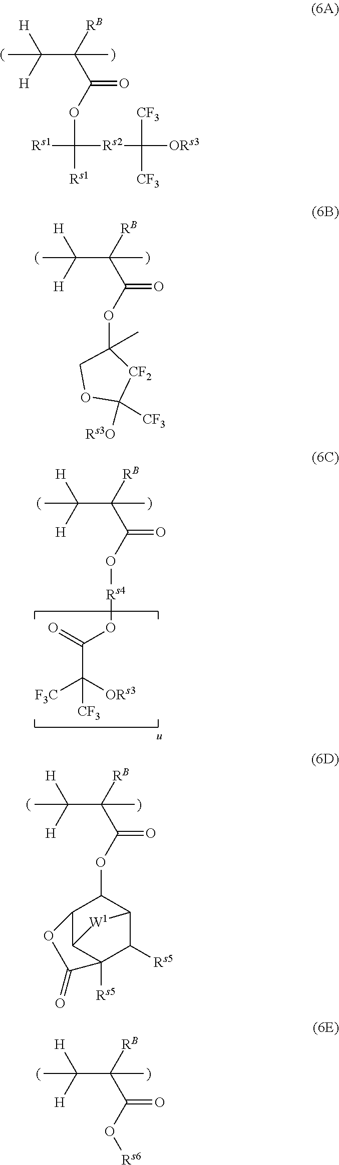

In the formulae (6A) to (6E), R^{B} represents a hydrogen atom, a fluorine atom, a methyl group, or a trifluoromethyl group. W¹ represents —CH₂—, —CH₂CH₂—, —O—, or two —H groups that are separate from each other. Each R^{s1} independently represents a hydrogen atom or a hydrocarbyl group having 1 to 10 carbon atoms. R^{s2} represents a single bond or a linear or branched hydrocarbylene group having 1 to 5 carbon atoms. Each R^{s3} independently represents a hydrogen atom, a hydrocarbyl group or fluorinated hydrocarbyl group having 1 to 15 carbon atoms, or an acid-labile group. When R^{s3} is a hydrocarbyl group or a fluorinated hydrocarbyl group, the group may have an intervening ether bond or carbonyl group in a carbon-carbon bond. R^{s4} represents a (u+1)-valent hydrocarbon group or fluorinated hydrocarbon group having 1 to 20 carbon atoms. “u” represents 1, 2, or 3. Each R^{s5} independently represents a hydrogen atom or a group represented by —C(═O)—O—R^{sa}. R^{sa} represents a fluorinated hydrocarbyl group having 1 to 20 carbon atoms. R^{s6} represents a hydrocarbyl group or fluorinated hydrocarbyl group having 1 to 15 carbon atoms, and optionally has an intervening ether bond or carbonyl group in a carbon-carbon bond thereof.

The hydrocarbyl group represented by R^{s1} having 1 to 10 carbon atoms is preferably a saturated hydrocarbyl group, and may be linear, branched, or cyclic. Specific examples thereof include: alkyl groups having 1 to 10 carbon atoms, such as a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, an n-nonyl group, and an n-decyl group; and cyclic saturated hydrocarbyl groups having 3 to 10 carbon atoms, such as a cyclopropyl group, a cyclobutyl group, a cyclopentyl group, a cyclohexyl group, an adamantyl group, and a norbornyl group. Among these, groups having 1 to 6 carbon atoms are preferable.

The hydrocarbylene group represented by R^{s2} is preferably a saturated hydrocarbylene group, and may be linear, branched, or cyclic. Specific examples thereof include a methylene group, an ethylene group, a propylene group, a butylene group, and a pentylene group.

The hydrocarbyl group represented by R^{s3} or R^{s6} may be saturated or unsaturated, and may be linear, branched, or cyclic. Specific examples thereof include saturated hydrocarbyl groups and aliphatic unsaturated hydrocarbyl groups, such as alkenyl groups and alkynyl groups, and saturated hydrocarbyl groups are preferable. Specific examples of the saturated hydrocarbyl groups include, besides those given as examples of the hydrocarbyl group represented by R^{s1}, an undecyl group, a dodecyl group, a tridecyl group, a tetradecyl group, and a pentadecyl group. Specific examples of the fluorinated hydrocarbyl group represented by R^{s3} or R^{s6} include groups which are the above-described hydrocarbyl groups in which part or all of the hydrogen atoms bonded to the carbon atoms of the hydrocarbyl groups are substituted with fluorine atoms. As described above, the fluorinated hydrocarbyl group may have an intervening ether bond or carbonyl group in a carbon-carbon bond thereof.

Specific examples of the acid-labile group represented by R^{s3} include groups represented by the above-described formulae (AL-1) to (AL-3), trialkylsilyl groups in which each alkyl group has 1 to 6 carbon atoms, and oxo-group-containing alkyl groups having 4 to 20 carbon atoms.

The (u+1)-valent hydrocarbon group or fluorinated hydrocarbon group represented by R^{s4} may be linear, branched, or cyclic, and specific examples thereof include groups obtained by further “u” hydrogen atoms being removed from the above-described hydrocarbyl groups, fluorinated hydrocarbyl groups, etc.

The fluorinated hydrocarbyl group represented by R^{sa} is preferably a saturated group, and may be linear, branched, or cyclic. Specific examples thereof include groups in which part or all of the hydrogen atoms of the above hydrocarbyl group are substituted with fluorine atoms. Specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of any of the repeating units represented by the formulae (6A) to (6E) include the following, but are not limited thereto. Note that, in the following formulae, R^{B} is as defined above.

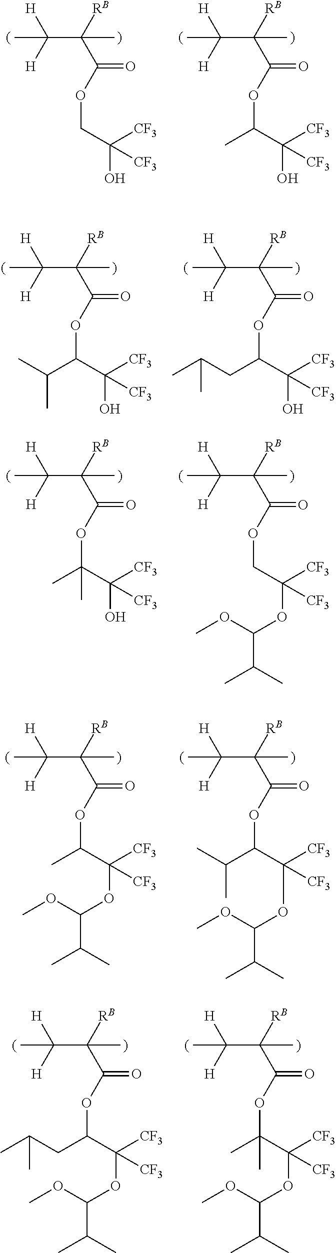

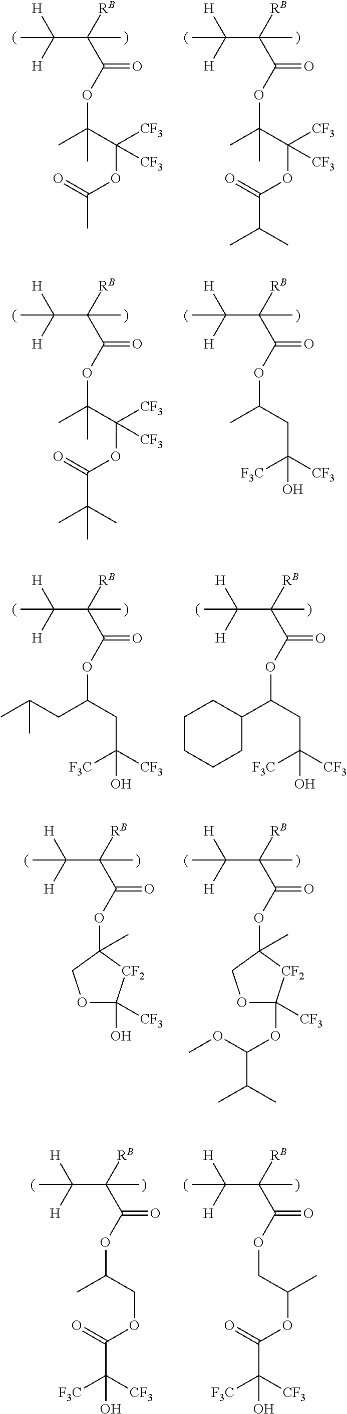

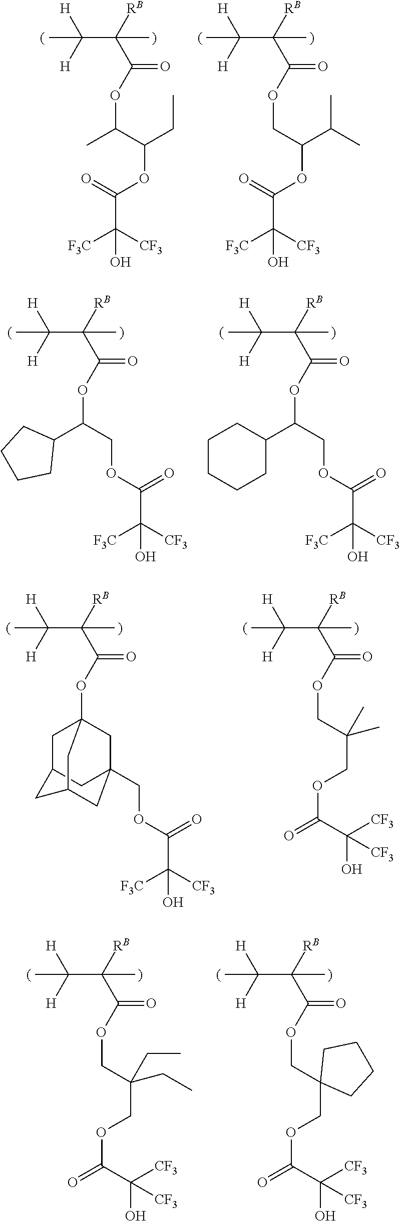

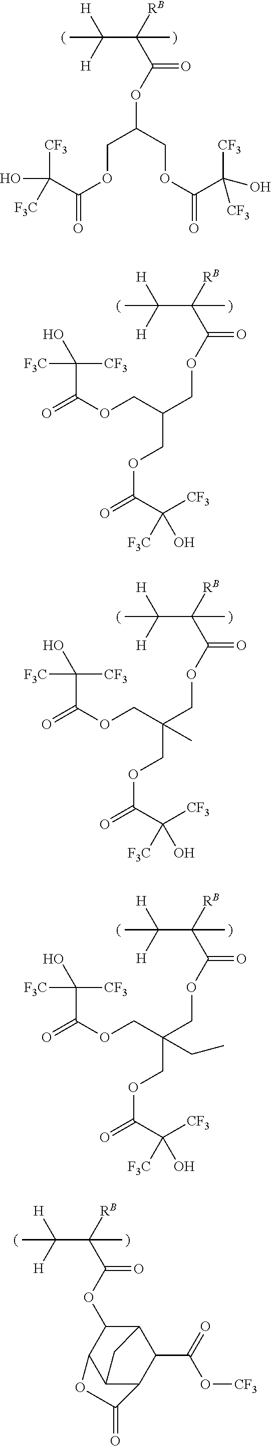

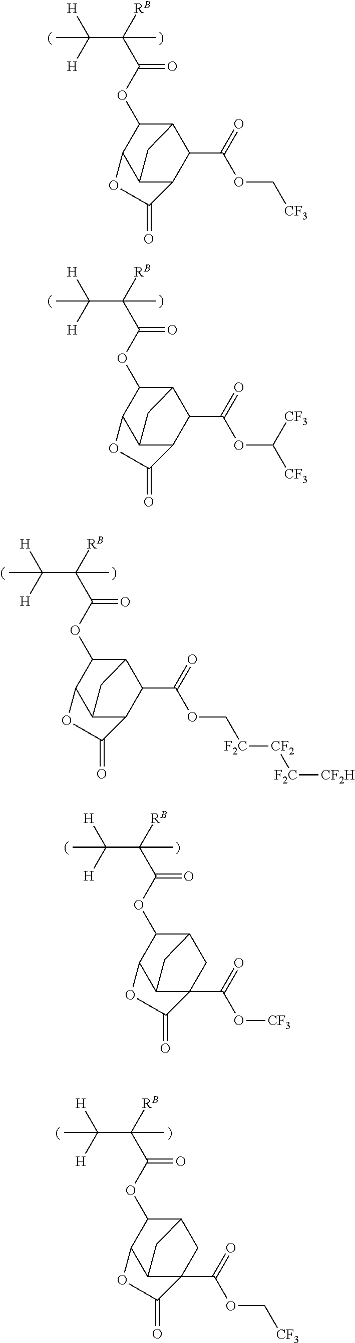

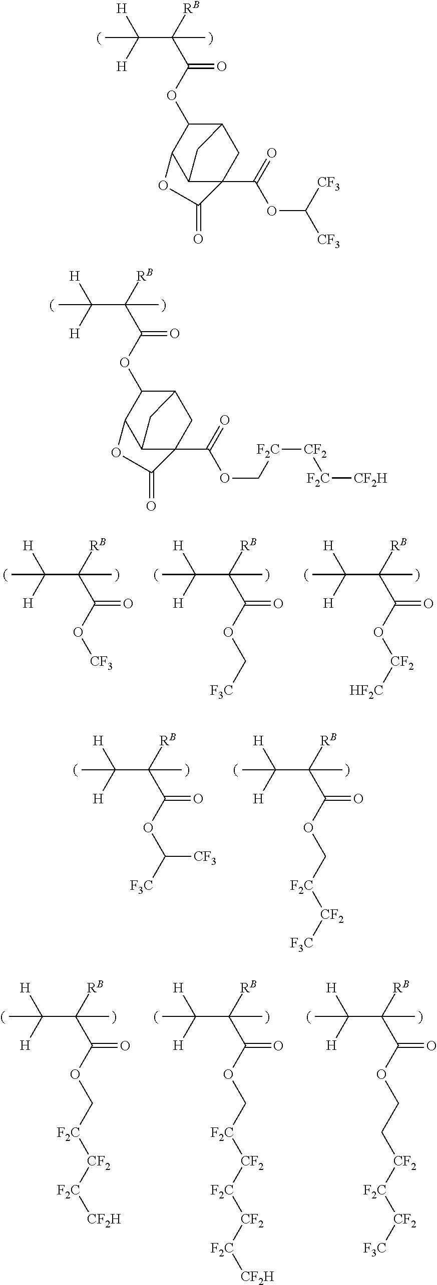

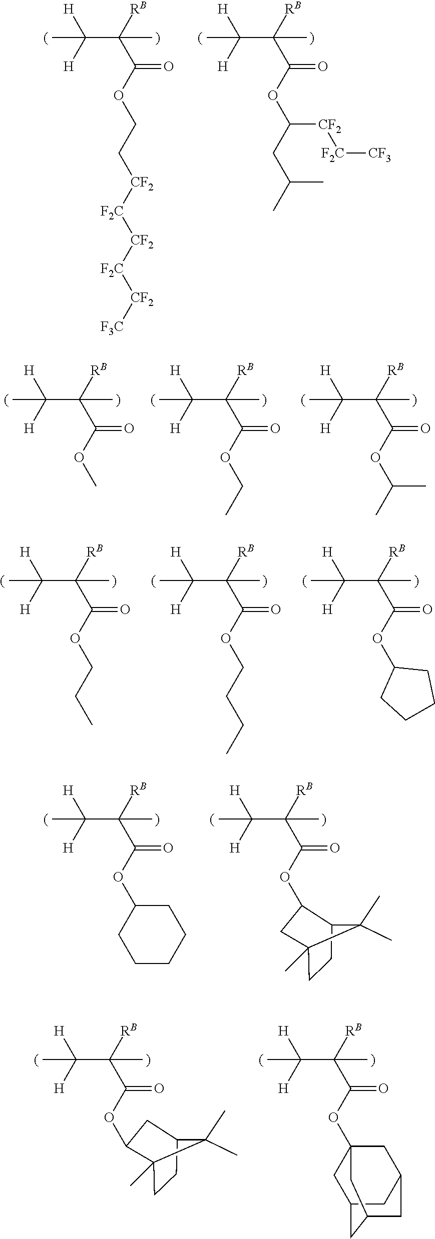

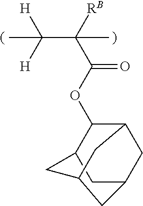

The polymer surfactant may further have a repeating unit other than the repeating unit represented by the formulae (6A) to (6E). Specific examples of the other repeating unit include repeating units obtained from methacrylic acid, an α-trifluoromethylacrylic acid derivative, etc. In the polymer surfactant, the contained amount of the repeating units represented by the formulae (6A) to (6E) is preferably 20 mol % or more, more preferably 60 mol % or more, and further preferably 100 mol % of all the repeating units.

The Mw of the polymer surfactant is preferably 1000 to 500000, and more preferably 3000 to 100000. Mw/Mn is preferably 1.0 to 2.0, more preferably 1.0 to 1.6.

Examples of a method for synthesizing the polymer surfactant include a method in which, in an organic solvent, monomers having an unsaturated bond to yield the repeating unit represented by the formulae (6A) to (6E) and, as necessary, other repeating units are added with a radical initiator and heated to be polymerized. Specific examples of the organic solvent used in the polymerization include toluene, benzene, THF, diethyl ether, and dioxane. Specific examples of the polymerization initiator include AIBN, 2,2′-azobis(2,4-dimethylvaleronitrile), dimethyl 2,2-azobis(2-methylpropionate), benzoyl peroxide, and lauroyl peroxide. The reaction temperature is preferably 50 to 100° C. The reaction time is preferably 4 to 24 hours. The acid-labile group may be introduced into the monomer to be used as it is, or may be protected or partially protected after the polymerization.

When the polymer surfactant is synthesized, known chain transfer agents, such as dodecyl mercaptan and 2-mercaptoethanol may be used to regulate the molecular weight. In this case, the addition amount of these chain transfer agents is preferably 0.01 to 10 mol % relative to the total number of moles of the monomers to be polymerized.

When the inventive chemically-amplified resist composition contains the surfactant (E), the contained amount is preferably 0.1 to 50 parts by mass, more preferably 0.5 to 10 parts by mass based on 80 parts by mass of the base polymer (A). When the contained amount of the surfactant (E) is 0.1 parts by mass or more, a sweepback contact angle between the resist film surface and water is sufficiently improved. When the contained amount is 50 parts by mass or less, the resist film surface has a low dissolution rate in the developer to sufficiently maintain the height of the formed fine pattern. One kind of the surfactant (E) may be used, or two or more kinds thereof may be used in combination.

### [(F) Dissolution Inhibitor]

The inventive chemically-amplified resist composition may further contain a dissolution inhibitor as a component (F). When the inventive chemically-amplified resist composition is a positive type, blending a dissolution inhibitor can further increase the difference in dissolution rate between exposed and unexposed portions, and further enhance the resolution.

Specific examples of the dissolution inhibitor include a compound which contains two or more phenolic hydroxy groups per molecule, and in which 0 to 100 mol % of all the hydrogen atoms of the phenolic hydroxy groups are substituted with acid-labile groups; and a compound which contains a carboxy group in a molecule, and in which 50 to 100 mol % of all the hydrogen atoms of such carboxy groups are substituted with acid-labile groups on average. The compounds each have a molecular weight of preferably 100 to 1000, more preferably 150 to 800. Specific examples include compounds obtained by substituting acid-labile groups for hydrogen atoms of hydroxy groups or carboxy groups of bisphenol A, trisphenol, phenolphthalein, cresol novolak, naphthalenecarboxylic acid, adamantanecarboxylic acid, or cholic acid; etc. Examples of such compounds include those disclosed in paragraphs [0155] to [0178] of JP 2008-122932 A.

When the inventive chemically-amplified resist composition contains the dissolution inhibitor (F), the contained amount is preferably 0 to 50 parts by mass, more preferably 5 to 40 parts by mass based on 80 parts by mass of the base polymer (A). One kind of the dissolution inhibitor (F) can be used, or two or more kinds thereof can be used in combination.

### [(G) Other Components]

The inventive chemically-amplified resist composition may contain, as other components (G): compounds to be decomposed by an acid to generate an acid (acid amplifying compounds); organic acid derivatives; fluorine-substituted alcohols; water-repellency enhancers; etc. As the acid amplifying compound, compounds described in JP 2009-269953 A or JP 2010-215608 A can be referred to. When the acid amplifying compound is contained, the contained amount is preferably 0 to 5 parts by mass, more preferably 0 to 3 parts by mass, based on 80 parts by mass of the base polymer (A). When the contained amount is within the above range, the acid diffusion is easily controlled, and degradation of resolution and degradation of the pattern profile can be suppressed. As the organic acid derivative and the fluorine-substituted alcohol, compounds described in JP 2009-269953 A or JP 2010-215608 A can be referred to.

The water-repellency enhancer can be employed in immersion lithography with no top coat. The water-repellency enhancer is preferably a polymer containing a fluorinated alkyl group, a polymer containing a 1,1,1,3,3,3-hexafluoro-2-propanol residue with a particular structure, etc., more preferably ones exemplified in JP 2007-297590 A, JP 2008-111103 A, etc. The water-repellency enhancer needs to be dissolved in an alkali developer or an organic solvent developer. The water-repellency enhancer having a particular 1,1,1,3,3,3-hexafluoro-2-propanol residue mentioned above has favorable solubility to developers. A polymer containing a repeating unit containing an amino group or amine salt as a water-repellency enhancer exhibits high effects of preventing acid evaporation during PEB and opening failure of a hole pattern after development. When the inventive chemically-amplified resist composition contains the water-repellency enhancer, the contained amount is preferably 0 to 20 parts by mass, more preferably 0.5 to 10 parts by mass based on 80 parts by mass of the base polymer (A).

### [Patterning Process]

When the inventive chemically-amplified resist composition is used for manufacturing various integrated circuits, known lithography techniques can be applied. Examples of the patterning process include a method including the steps of:
forming a resist film by using the above-described chemically-amplified resist composition on a substrate;
exposing the resist film by using a high-energy beam; and
developing the exposed resist film by using a developer.

Firstly, the inventive chemically-amplified resist composition is applied onto a substrate (such as Si, SiO₂, SiN, SiON, TiN, WSi, BPSG, SOG, or organic antireflective film) for manufacturing an integrated circuit or a substrate (such as Cr, CrO, CrON, MoSi₂, or SiO₂) for manufacturing a mask circuit by an appropriate coating process, such as spin coating, roll coating, flow coating, dip coating, spray coating, or doctor coating, so that the coating film has a thickness of 0.01 to 2.0 μm. The resultant is prebaked on a hot plate preferably at 60 to 150° C. for 10 seconds to 30 minutes, more preferably at 80 to 120° C. for 30 seconds to 20 minutes. In this manner, a resist film is formed.

Then, the resist film is exposed using a high-energy beam. Examples of the high-energy beam include ultraviolet ray, deep ultraviolet ray, EB, EUV having a wavelength of 3 to 15 nm, X-ray, soft X-ray, excimer laser beam, γ-ray, synchrotron radiation, etc. When ultraviolet ray, deep ultraviolet ray, EUV, X-ray, soft X-ray, excimer laser beam, γ-ray, synchrotron radiation, or the like is employed as the high-energy beam, the irradiation is performed directly or while using a mask for forming a target pattern at an exposure dose of preferably about 1 to 200 mJ/cm², more preferably about 10 to 100 mJ/cm². When EB is employed as the high-energy beam, the exposure dose is preferably about 0.1 to 100 μC/cm², more preferably about 0.5 to 50 μC/cm², and the writing is performed directly or while using a mask for forming a target pattern. The inventive chemically-amplified resist composition is particularly suitable for fine patterning with an ArF excimer laser beam having a wavelength of 193 nm, a KrF excimer laser beam having a wavelength of 248 nm, an EB (electron beam), an EUV (extreme ultraviolet ray) having a wavelength of 3 to 15 nm, X-ray, soft X-ray, γ-ray, or synchrotron radiation among the high-energy beams.

The exposure may be followed by PEB on a hot plate, preferably at 60 to 150° C. for 10 seconds to 30 minutes, more preferably 80 to 120° C. for 30 seconds to 20 minutes.

After the exposure or PEB, development is performed using a developer of 0.1 to 10 mass %, preferably 2 to 5 mass %, aqueous alkaline solution, such as tetramethylammonium hydroxide (TMAH), tetraethylammonium hydroxide, tetrapropylammonium hydroxide, and tetrabutylammonium hydroxide, for 3 seconds to 3 minutes, preferably 5 seconds to 2 minutes, by a conventional technique, such as a dip, puddle, or spray method. Thereby, the portion irradiated with the light is dissolved by the developer, while the unexposed portion remains undissolved. In this way, the target positive pattern is formed on the substrate.

It is also possible to obtain a negative pattern by using an organic solvent developer instead of the aqueous alkaline solution. Specific examples of the developer used in this event include 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, benzyl formate, phenylethyl formate, methyl 3-phenylpropionate, benzyl propionate, phenylethyl acetate, 2-phenylethyl acetate, etc. One of these organic solvents can be used, or two or more thereof can be used in mixture.

When the development is completed, rinsing can be performed. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, and an alkane, alkene, alkyne, and aromatic solvent, each having 6 to 12 carbon atoms.

Specific examples of the alcohol having 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, 1-octanol, etc.

Specific examples of the ether compound having 8 to 12 carbon atoms include di-n-butyl ether, diisobutyl ether, di-sec-butyl ether, di-n-pentyl ether, diisopentyl ether, di-sec-pentyl ether, di-tert-pentyl ether, di-n-hexyl ether, etc.

Specific examples of the alkane having 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, cyclononane, etc. Specific examples of the alkene having 6 to 12 carbon atoms include hexene, heptene, octene, cyclohexene, methylcyclohexene, dimethylcyclohexene, cycloheptene, cyclooctene, etc. Specific examples of the alkyne having 6 to 12 carbon atoms include hexyne, heptyne, octyne, etc.

Specific examples of the aromatic solvent include toluene, xylene, ethylbenzene, isopropylbenzene, tert-butylbenzene, mesitylene, etc.

The rinsing can reduce resist pattern collapse and defect formation. Meanwhile, the rinsing is not necessarily essential, and the amount of the solvent used can be reduced by not performing the rinsing.

After the development, a hole pattern or trench pattern can be shrunk by thermal flow, RELACS process, or DSA process. A shrink agent is applied onto the hole pattern, and the shrink agent undergoes crosslinking on the resist film surface by diffusion of the acid catalyst from the resist film during baking, so that the shrink agent is attached to sidewalls of the hole pattern. The baking temperature is preferably 70 to 180° C., more preferably 80 to 170° C. The baking time is preferably 10 to 300 seconds. The extra shrink agent is removed, and thus the hole pattern is shrunk.

### EXAMPLES

Hereinafter, the present invention will be specifically described with reference to Synthesis Examples, Examples, and Comparative Examples, but the present invention is not limited to the following Examples. Incidentally, the apparatus used is as follows.
MALDI TOF-MS: S3000 manufactured by JEOL Ltd.

### [1] Synthesis of Onium-Salt-Type Monomers

### [Example 1-1] Synthesis of Onium-Salt-Type Monomer a-1

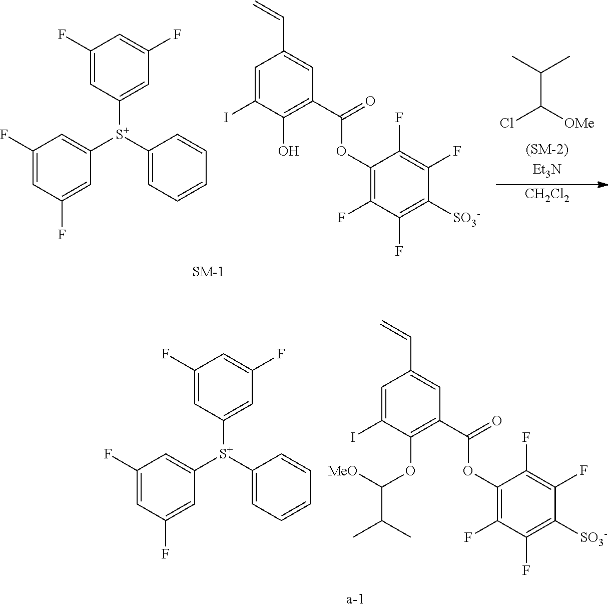

Under a nitrogen atmosphere, a raw material SM-1 (42.5 g) and triethylamine (6.1 g) were dissolved in methylene chloride (200 g) in a reaction container, and the mixture was cooled in an ice bath. While maintaining the temperature inside the reaction container at 20° C. or lower, a raw material SM-2 (6.7 g) was added dropwise. After the addition, the temperature was raised to room temperature, and the mixture was aged for 4 hours. After the aging, the reaction container was cooled in an ice bath, water was added to terminate the reaction, a common aqueous work-up was performed, the solvent was distilled off, and then, diisopropyl ether was added to wash the residue. Thus, 44.1 g of onium-salt-type monomer a-1 was obtained as an oil (94% yield).

### MALDI TOF-MS:

POSITIVE M⁺335 (C₁₈H₁₁F₄S⁺ equivalent)
NEGATIVE M⁻603 (C₂₀H₁₆F₄IO₇S⁻ equivalent)

### [Examples 1-2 to 1-8] Synthesis of Onium-Salt-Type Monomers a-2 to a-8

Onium-salt-type monomers a-2 to a-8, represented by the following formulae, were synthesized using corresponding raw materials by using known organic synthesis reactions.

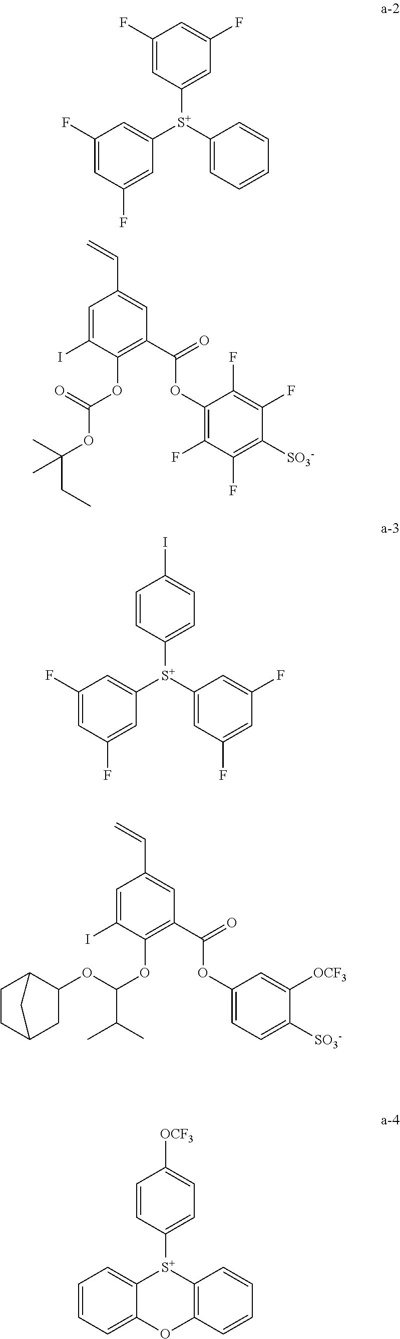

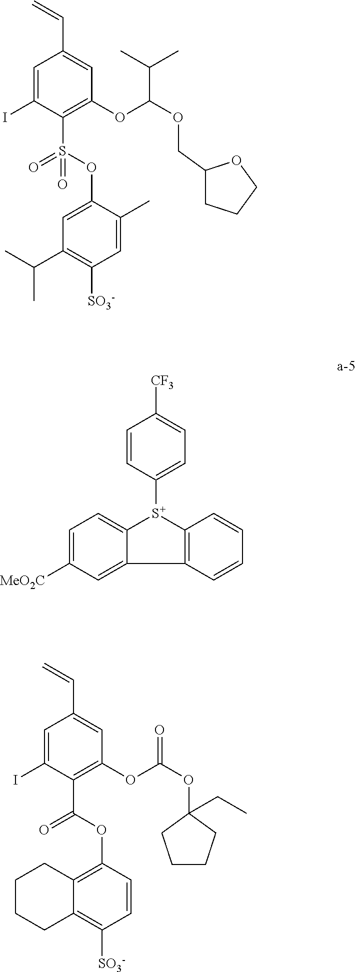

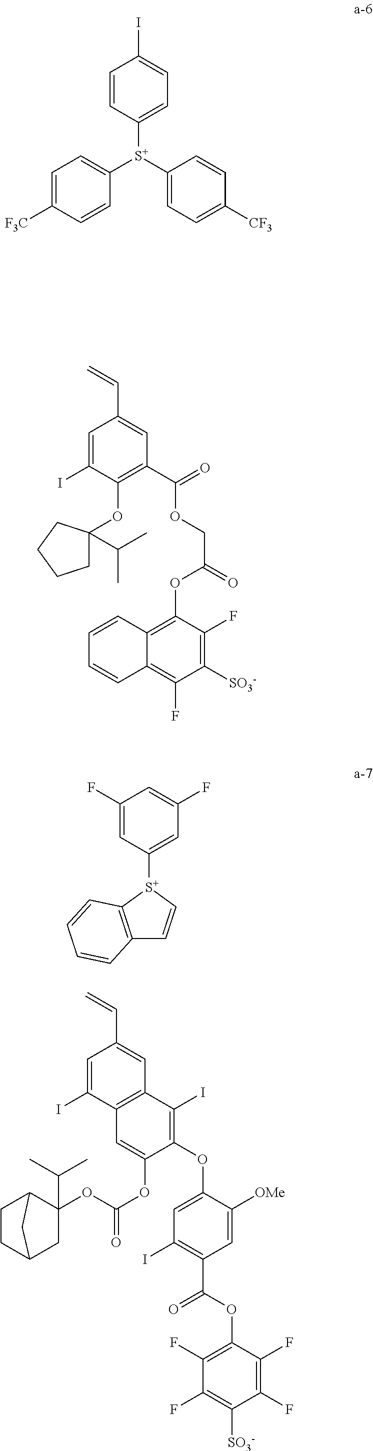

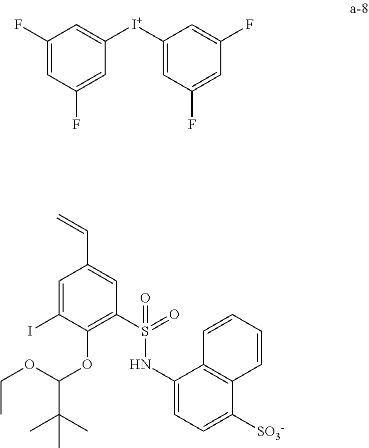

### [Comparative Examples 1-1 to 1-4] Synthesis of Comparative Onium-Salt-Type Monomers ca-1 to ca-4

Comparative onium-salt-type monomers ca-1 to ca-4, represented by the following formulae, were synthesized using corresponding raw materials by using known organic synthesis reactions.

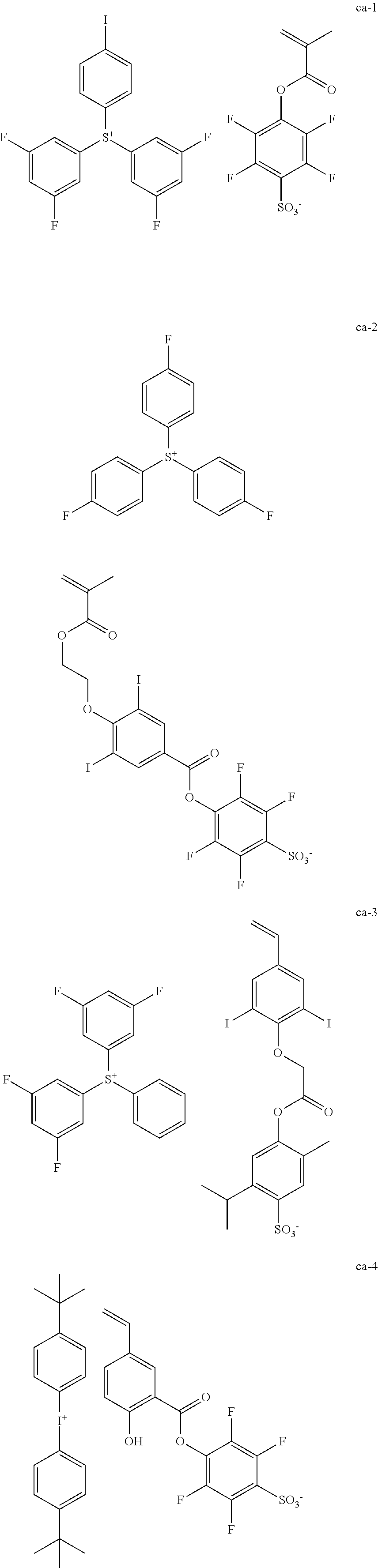

### [2] Synthesis of Base Polymers

Among the monomers used for synthesizing base polymers, monomers other than monomers a-1 to a-8 and comparative monomers ca-1 to ca-4 are as follows.

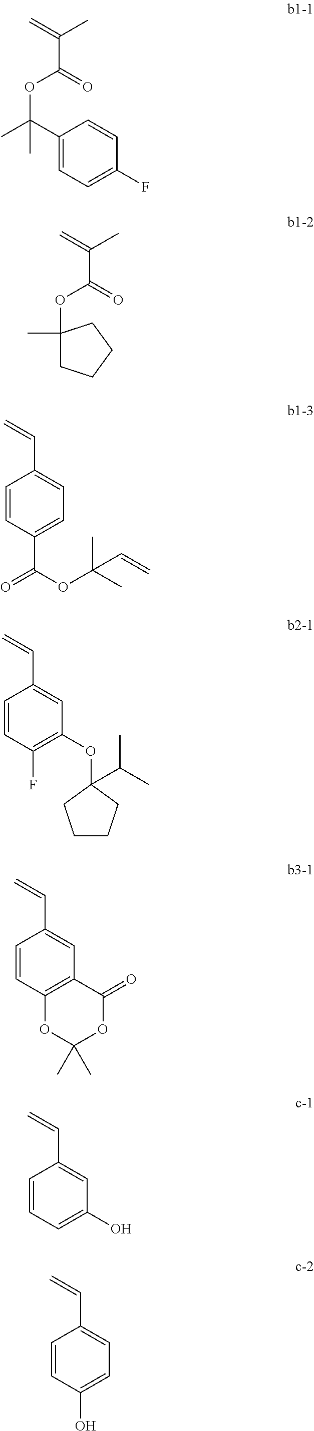

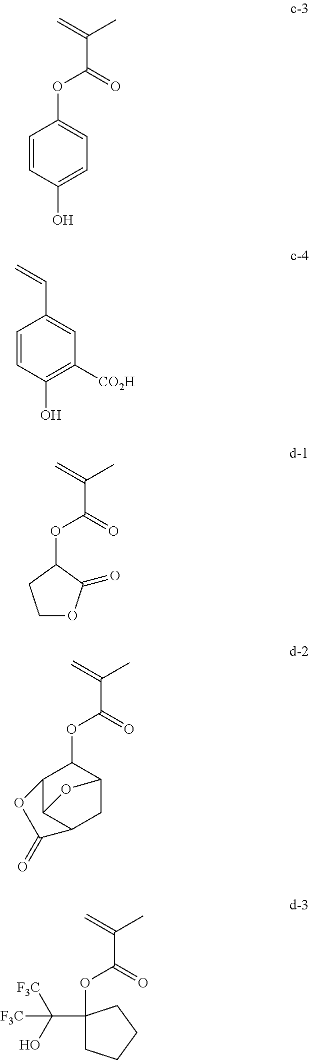

### [Example 2-1] Synthesis of Polymer P-1

Under a nitrogen atmosphere, monomer a-1 (23.5 g), monomer b1-1 (20.5 g), monomer c-1 (6.0 g), 1.92 g of V-601 (manufactured by FUJIFILM Wako Pure Chemical Corporation), and 70 g of MEK were added into a flask to prepare a monomer-polymerization initiator solution. Into another flask with a nitrogen atmosphere, 23 g of MEK was added and heated to 80° C. with stirring, and then the monomer-polymerization initiator solution was added dropwise over 4 hours. After the dropwise addition, the polymerization liquid was further stirred for 2 hours with maintaining the temperature at 80° C., and then cooled to room temperature. The obtained polymerization liquid was added dropwise to 1500 g of vigorously stirred hexane, and a precipitated polymer was filtered. The obtained polymer was washed twice with 300 g of hexane, and then dried in vacuo at 50° C. for 20 hours to obtain a white powder polymer P-1 (48.1 g, 96% yield). The polymer P-1 had Mw of 9300 and Mw/Mn of 1.61. The Mw is a polystyrene-converted measurement value obtained by GPC using DMF as an eluent.

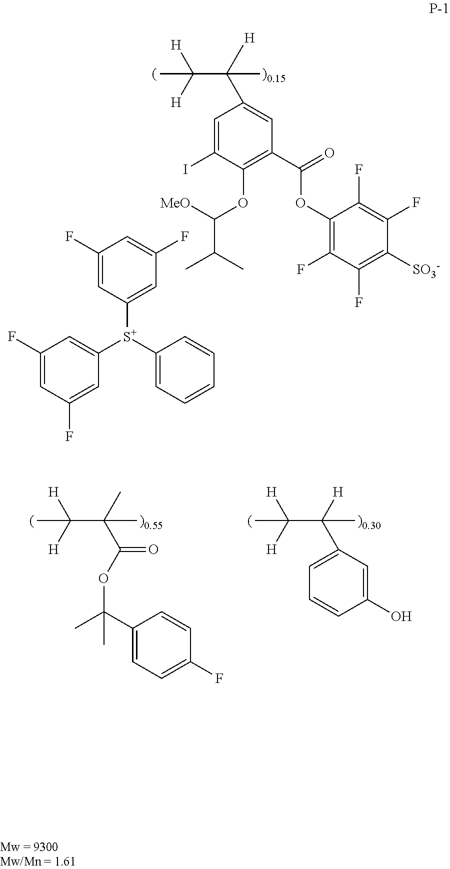

### [Examples 2-2 to 2-21 and Comparative Examples 2-1 to 2-11] Synthesis of Polymers P-2 to P-21 and Comparative Polymers CP-1 to CP-11

The polymers shown in Tables 1 and 2 were produced in the same manner as in Example 2-1 except that the kinds and blending ratios of the monomers were changed.

TABLE 1

| | | Introduction | | Introduction | | Introduction | | Introduction | | Introduction | | |
|---|---|---|---|---|---|---|---|---|---|---|---|---|
| | Unit | rate | Unit | rate | Unit | rate | Unit | rate | Unit | rate | | |
| Polymer | 1 | (mol %) | 2 | (mol %) | 3 | (mol %) | 4 | (mol %) | 5 | (mol %) | Mw | Mw/Mn |
| P-1 | a-1 | 15 | b1-1 | 55 | c-1 | 30 | — | — | — | — | 9300 | 1.61 |
| P-2 | a-2 | 15 | b1-1 | 55 | c-1 | 30 | — | — | — | — | 9200 | 1.62 |
| P-3 | a-3 | 15 | b1-1 | 55 | c-1 | 30 | — | — | — | — | 9100 | 1.62 |
| P-4 | a-4 | 15 | b1-1 | 55 | c-1 | 30 | — | — | — | — | 9000 | 1.61 |
| P-5 | a-5 | 15 | b1-1 | 55 | c-1 | 30 | — | — | — | — | 9300 | 1.63 |
| P-6 | a-6 | 15 | b1-1 | 55 | c-1 | 30 | — | — | — | — | 9200 | 1.64 |
| P-7 | a-7 | 15 | b1-1 | 55 | c-1 | 30 | — | — | — | — | 9100 | 1.61 |
| P-8 | a-8 | 15 | b1-1 | 55 | c-1 | 30 | — | — | — | — | 9200 | 1.62 |
| P-9 | a-1 | 15 | b1-2 | 55 | c-1 | 30 | — | — | — | — | 9300 | 1.63 |
| P-10 | a-1 | 15 | b1-3 | 55 | c-1 | 30 | — | — | — | — | 8900 | 1.64 |
| P-11 | a-2 | 15 | b2-1 | 55 | c-1 | 30 | — | — | — | — | 9000 | 1.61 |
| P-12 | a-3 | 10 | b1-3 | 35 | b3-1 | 20 | c-2 | 25 | d-1 | 10 | 9100 | 1.62 |
| P-13 | a-4 | 15 | b1-1 | 25 | b1-2 | 25 | c-2 | 35 | — | — | 9400 | 1.63 |
| P-14 | a-2 | 15 | b1-3 | 50 | c-3 | 25 | d-2 | 10 | — | — | 9000 | 1.61 |
| P-15 | a-6 | 15 | b2-1 | 25 | b3-1 | 25 | c-4 | 35 | — | — | 9100 | 1.63 |
| P-16 | a-7 | 10 | b1-2 | 35 | b3-1 | 15 | c-2 | 30 | d-3 | 10 | 8900 | 1.62 |
| P-17 | a-1 | 15 | b1-1 | 35 | b1-3 | 15 | c-4 | 35 | — | — | 9300 | 1.64 |
| P-18 | a-2 | 15 | b1-1 | 25 | b3-1 | 25 | c-2 | 20 | d-1 | 15 | 9200 | 1.61 |
| P-19 | a-3 | 20 | b1-2 | 45 | c-2 | 30 | d-3 | 5 | — | — | 8800 | 1.62 |
| P-20 | a-4 | 5 | b1-1 | 50 | c-2 | 25 | d-2 | 10 | d-3 | 10 | 9200 | 1.63 |
| P-21 | a-1 | 15 | b1-2 | 30 | b1-3 | 20 | c-2 | 35 | — | — | 9100 | 1.64 |

TABLE 2

| | | Introduction | | Introduction | | Introduction | | Introduction | | Introduction | | |
|---|---|---|---|---|---|---|---|---|---|---|---|---|
| | Unit | rate | Unit | rate | Unit | rate | Unit | rate | Unit | rate | | |
| Polymer | 1 | (mol %) | 2 | (mol %) | 3 | (mol %) | 4 | (mol %) | 5 | (mol %) | Mw | Mw/Mn |
| CP-1 | ca-1 | 15 | b1-1 | 55 | c-1 | 30 | — | — | — | — | 9500 | 1.62 |
| CP-2 | ca-2 | 15 | b1-1 | 55 | c-1 | 30 | — | — | — | — | 9100 | 1.61 |
| CP-3 | ca-3 | 15 | b1-1 | 55 | c-1 | 30 | — | — | — | — | 9200 | 1.63 |
| CP-4 | ca-4 | 15 | b1-1 | 55 | c-1 | 30 | — | — | — | — | 8900 | 1.61 |
| CP-5 | ca-1 | 10 | b1-3 | 35 | b3-1 | 20 | c-2 | 25 | d-1 | 10 | 9000 | 1.62 |
| CP-6 | ca-2 | 10 | b1-2 | 35 | b3-1 | 15 | c-2 | 30 | d-3 | 10 | 9200 | 1.62 |
| CP-7 | ca-3 | 15 | b1-1 | 35 | b1-3 | 15 | c-4 | 35 | — | — | 9100 | 1.64 |
| CP-8 | ca-4 | 15 | b1-3 | 50 | c-3 | 25 | d-2 | 10 | — | — | 9300 | 1.61 |
| CP-9 | ca-2 | 15 | b1-1 | 25 | b3-1 | 25 | c-2 | 20 | d-1 | 15 | 9200 | 1.62 |
| CP-10 | ca-3 | 5 | b1-1 | 50 | c-2 | 25 | d-2 | 10 | d-3 | 10 | 9200 | 1.63 |
| CP-11 | ca-4 | 15 | b1-2 | 55 | c-1 | 30 | — | — | — | — | 9200 | 1.62 |

### [3] Preparation of Chemically-Amplified Resist Compositions

### Examples 3-1 to 3-21 and Comparative Examples 3-1 to 3-11

Prepared was a solution of predetermined components selected from the base polymers (P-1 to P-21) of the present invention, the comparative base polymers (CP-1 to CP-11), acid generators (PAG-1 and PAG-2), and quenchers (SQ-1 to SQ-3 and AQ-1) at the constitution shown in the following Tables 3 and 4 by dissolving the components in a solvent containing 0.01 mass % of FC-4430, manufactured by 3M Company, as a surfactant. The solution was filtered with a 0.2-μm Teflon (registered trademark) type filter to prepare a chemically-amplified resist composition (R-1 to R-21 and CR-1 to CR-11).

TABLE 3

| | | Base | | Acid | | | |
|---|---|---|---|---|---|---|---|
| | | polymer | Quencher | generator | Solvent 1 | Solvent 2 | Solvent 3 |
| | Resist | (parts | (parts | (parts | (parts | (parts | (parts |
| | composition | by mass) | by mass) | by mass) | by mass) | by mass) | by mass) |
| Example | R-1 | P-1(80) | SQ-1(8.0) | — | PGMEA(2250) | EL(2800) | DAA(550) |
| 3-1 | | | | | | | |
| Example | R-2 | P-2(80) | SQ-1(7.8) | — | PGMEA(2250) | EL(2800) | DAA(550) |
| 3-2 | | | | | | | |
| Example | R-3 | P-3(80) | SQ-1(7.4) | — | PGMEA(2250) | EL(2800) | DAA(550) |
| 3-3 | | | | | | | |
| Example | R-4 | P-4(80) | SQ-1(8.0) | — | PGMEA(2250) | EL(2800) | DAA(550) |
| 3-4 | | | | | | | |
| Example | R-5 | P-5(80) | SQ-1(8.0) | — | PGMEA(2250) | EL(2800) | DAA(550) |
| 3-5 | | | | | | | |
| Example | R-6 | P-6(80) | SQ-1(8.0) | — | PGMEA(2250) | EL(2800) | DAA(550) |
| 3-6 | | | | | | | |
| Example | R-7 | P-7(80) | SQ-1(7.8) | — | PGMEA(2250) | EL(2800) | DAA(550) |
| 3-7 | | | | | | | |
| Example | R-8 | P-8(80) | SQ-1(7.8) | — | PGMEA(2250) | EL(2800) | DAA(550) |
| 3-8 | | | | | | | |
| Example | R-9 | P-9(80) | SQ-2(8.0) | — | PGMEA(2250) | EL(2800) | DAA(550) |
| 3-9 | | | | | | | |
| Example | R-10 | P-10(80) | SQ-2(8.0) | — | PGMEA(2250) | EL(2800) | DAA(550) |
| 3-10 | | | | | | | |
| Example | R-11 | P-11(80) | SQ-1(4.0)/ | — | PGMEA(2250) | EL(2800) | DAA(550) |
| 3-11 | | | AQ-1(4.0) | | | | |
| Example | R-12 | P-12(80) | SQ-3(7.8) | — | PGMEA(2250) | EL(2800) | DAA(550) |
| 3-12 | | | | | | | |
| Example | R-13 | P-13(80) | SQ-3(7.6) | — | PGMEA(2250) | EL(2800) | DAA(550) |
| 3-13 | | | | | | | |
| Example | R-14 | P-14(80) | SQ-1(7.8) | — | PGMEA(2250) | EL(2800) | DAA(550) |
| 3-14 | | | | | | | |
| Example | R-15 | P-15(80) | SQ-3(8.0) | — | PGMEA(2250) | EL(2800) | DAA(550) |
| 3-15 | | | | | | | |
| Example | R-16 | P-16(80) | SQ-2(4.0)/ | PAG-1(10) | PGMEA(2250) | EL(2800) | DAA(550) |
| 3-16 | | | AQ-1(4.0) | | | | |
| Example | R-17 | P-17(80) | SQ-1(8.2) | — | PGMEA(2250) | EL(2800) | DAA(550) |
| 3-17 | | | | | | | |
| Example | R-18 | P-18(80) | SQ-1(7.6) | — | PGMEA(2250) | EL(2800) | DAA(550) |
| 3-18 | | | | | | | |
| Example | R-19 | P-19(80) | SQ-3(8.0) | — | PGMEA(2250) | EL(2800) | DAA(550) |
| 3-19 | | | | | | | |
| Example | R-20 | P-20(80) | SQ-3(4.0)/ | PAG-2(15) | PGMEA(2250) | EL(2800) | DAA(550) |
| 3-20 | | | AQ-1(4.0) | | | | |
| Example | R-21 | P-21(80) | SQ-2(7.6) | — | PGMEA(2250) | EL(2800) | DAA(550) |
| 3-21 | | | | | | | |

TABLE 4

| | | Base | | Acid | | | |
|---|---|---|---|---|---|---|---|
| | | polymer | Quencher | generator | Solvent 1 | Solvent 2 | Solvent 3 |
| | Resist | (parts | (parts | (parts | (parts | (parts | (parts |
| | composition | by mass) | by mass) | by mass) | by mass) | by mass) | by mass) |
| Comparative | CR-1 | CP-1(80) | SQ-1(8.0) | — | PGMEA(2250) | EL(2800) | DAA(550) |
| Example 3-1 | | | | | | | |
| Comparative | CR-2 | CP-2(80) | SQ-1(7.8) | — | PGMEA(2250) | EL(2800) | DAA(550) |
| Example 3-2 | | | | | | | |
| Comparative | CR-3 | CP-3(80) | SQ-1(7.8) | — | PGMEA(2250) | EL(2800) | DAA(550) |
| Example 3-3 | | | | | | | |
| Comparative | CR-4 | CP-4(80) | SQ-1(8.2) | — | PGMEA(2250) | EL(2800) | DAA(550) |
| Example 3-4 | | | | | | | |
| Comparative | CR-5 | CP-5(80) | SQ-3(7.8) | — | PGMEA(2250) | EL(2800) | DAA(550) |
| Example 3-5 | | | | | | | |
| Comparative | CR-6 | CP-6(80) | SQ-2(4.0)/ | PAG-1(10) | PGMEA(2250) | EL(2800) | DAA(550) |
| Example 3-6 | | | AQ-1(4.0) | | | | |
| Comparative | CR-7 | CP-7(80) | SQ-1(8.2) | — | PGMEA(2250) | EL(2800) | DAA(550) |
| Example 3-7 | | | | | | | |
| Comparative | CR-8 | CP-8(80) | SQ-2(7.8) | — | PGMEA(2250) | EL(2800) | DAA(550) |
| Example 3-8 | | | | | | | |
| Comparative | CR-9 | CP-9(80) | SQ-3(7.6) | — | PGMEA(2250) | EL(2800) | DAA(550) |
| Example 3-9 | | | | | | | |
| Comparative | CR-10 | CP-10(80) | SQ-3(4.0)/ | PAG-2(15) | PGMEA(2250) | EL(2800) | DAA(550) |
| Example 3-10 | | | AQ-1(4.0) | | | | |
| Comparative | CR-11 | CP-11(80) | SQ-2(8.0) | — | PGMEA(2250) | EL(2800) | DAA(550) |
| Example 3-11 | | | | | | | |

In Tables 3 and 4, the solvents, the quenchers (SQ-1 to SQ-3 and AQ-1), and the acid generators (PAG-1 and PAG-2) are as follows.

### Solvents:

PGMEA (propylene glycol monomethyl ether acetate)
EL (ethyl lactate)
DAA (diacetone alcohol)

### Quenchers: SQ-1 to SQ-3 and AQ-1

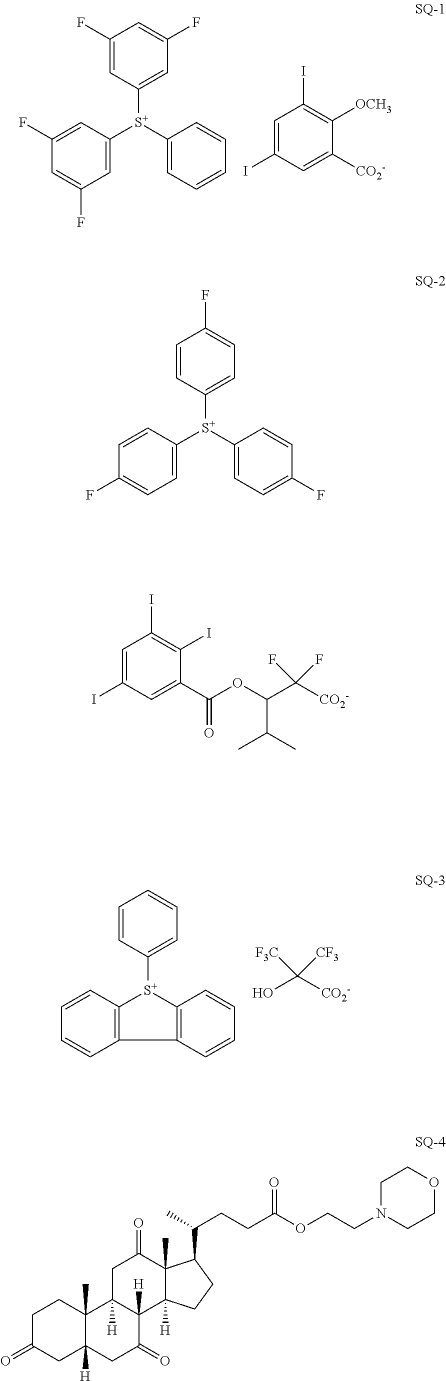

Acid generator: PAG-1 and PAG-2

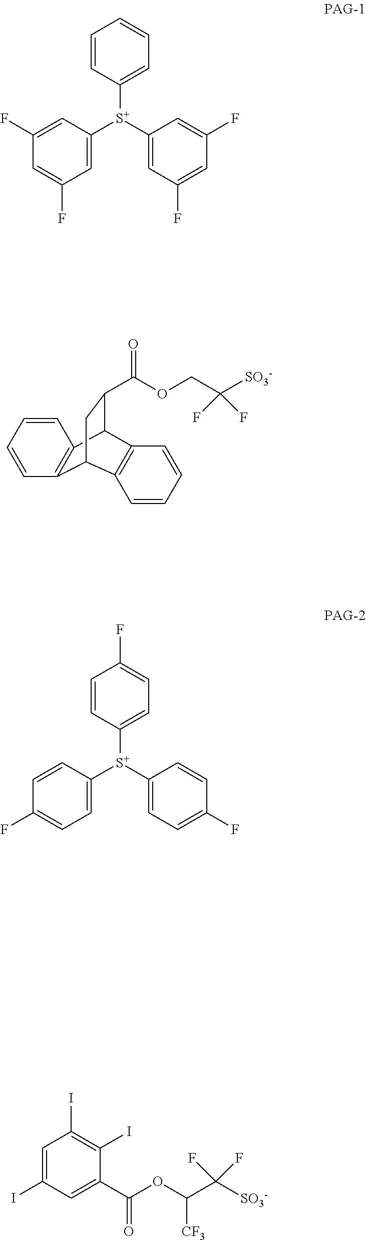

### [4] EUV Lithography Evaluation (1)

### Examples 4-1 to 4-21 and Comparative Examples 4-1 to 4-11

Each of the chemically-amplified resist compositions (R-1 to R-21 and CR-1 to CR-11) shown in Tables 3 and 4 was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 100° C. for 60 seconds using a hot plate to produce a resist film having a film thickness of 50 nm. This resist film was exposed using an EUV scanner NXE3300 (NA 0.33, σ 0.9/0.6, dipole illumination), manufactured by ASML Holding N.V. The exposure was performed with an LS pattern with 18 nm on wafer size and 36 nm in pitch, and with changing an exposure dose and focus (exposure dose pitch: 1 mJ/cm², focus pitch: 0.020 μm). After the exposure, PEB was performed at a temperature shown in Tables 5 and 6 for 60 seconds. Thereafter, puddle development with a 2.38 mass % aqueous TMAH solution for 30 seconds, rinse with a surfactant-containing rinse material, and spin-drying were performed to obtain a positive pattern.

The obtained LS pattern was observed with a length-measurement SEM (CG6300), manufactured by Hitachi High-Technologies Corporation, to evaluate sensitivity, EL, LWR, DOF, and collapse limit in accordance with the following methods. Tables 5 and 6 show the results.

### [Sensitivity Evaluation]

An optimum exposure dose Eₒₚ(mJ/cm²) to yield the LS pattern with 18 nm in line width and 36 nm in pitch was determined to specify this value as sensitivity. The smaller this value, the higher the sensitivity.

### [EL Evaluation]

From exposure doses to form the LS pattern within a range of ±10% of 18 nm space width (16.2 to 19.8 nm), EL (unit: %) was determined by the following equation. The larger the EL value, the better the performance.

$$EL ( \% ) = ( \vert {E}_{1} - {E}_{2} \vert / {E}_{op} ) \times 100$$

E₁: An optimum exposure dose to yield an LS pattern with 16.2 nm in line width and 36 nm in pitch.
E₂: An optimum exposure dose to yield an LS pattern with 19.8 nm in line width and 36 nm in pitch.
Eₒₚ: An optimum exposure dose to yield the LS pattern with 18 nm in line width and 36 nm in pitch.

### [LWR Evaluation]

In the LS pattern obtained by irradiation at Eₒₚ, sizes in 10 positions in the longitudinal direction of the line were measured. From the results, a tripled value (3σ) of a standard variation (σ) was determined as LWR. A smaller LWR value can yield a pattern with smaller roughness and uniform line width.

### [DOF Evaluation]

As evaluation of depth of focus, determined was a focus range to form the LS pattern within a range of ±10% of 18 nm size (16.2 to 19.8 nm). A larger DOF value indicates wider depth of focus.

### [Collapse Limit Evaluation of Line Pattern]

The line size of the LS pattern for each exposure dose at the optimum focus was measured in 10 positions in the longitudinal direction. The thinnest line size obtained without a collapse was obtained as the collapse limit. The smaller the collapse limit value, the better the collapse limit.

TABLE 5

| | | PEB | Optimum | | | | Collapse |
|---|---|---|---|---|---|---|---|
| | Resist | temperature | exposure dose | EL | LWR | DOF | limit |
| | composition | (° C.) | (mJ/cm²) | (%) | (nm) | (nm) | (nm) |
| Example 4-1 | R-1 | 100 | 32 | 19 | 2.4 | 120 | 10.7 |
| Example 4-2 | R-2 | 100 | 32 | 18 | 2.4 | 120 | 10.7 |
| Example 4-3 | R-3 | 100 | 32 | 18 | 2.6 | 110 | 10.8 |
| Example 4-4 | R-4 | 100 | 33 | 17 | 2.5 | 110 | 11 |
| Example 4-5 | R-5 | 105 | 34 | 19 | 2.4 | 110 | 11.1 |
| Example 4-6 | R-6 | 100 | 34 | 18 | 2.4 | 100 | 11.2 |
| Example 4-7 | R-7 | 95 | 35 | 17 | 2.5 | 120 | 11.2 |
| Example 4-8 | R-8 | 105 | 33 | 17 | 2.4 | 110 | 11.1 |
| Example 4-9 | R-9 | 100 | 32 | 18 | 2.6 | 100 | 11.1 |
| Example 4-10 | R-10 | 95 | 33 | 17 | 2.7 | 110 | 10.9 |
| Example 4-11 | R-11 | 100 | 33 | 19 | 2.5 | 120 | 10.8 |
| Example 4-12 | R-12 | 100 | 34 | 17 | 2.5 | 100 | 11.1 |
| Example 4-13 | R-13 | 105 | 32 | 18 | 2.6 | 110 | 10.8 |
| Example 4-14 | R-14 | 100 | 33 | 17 | 2.4 | 120 | 11.2 |
| Example 4-15 | R-15 | 100 | 34 | 19 | 2.5 | 110 | 11 |
| Example 4-16 | R-16 | 105 | 33 | 17 | 2.7 | 120 | 11.2 |
| Example 4-17 | R-17 | 95 | 32 | 18 | 2.5 | 120 | 11.3 |
| Example 4-18 | R-18 | 100 | 34 | 18 | 2.4 | 110 | 11.2 |
| Example 4-19 | R-19 | 100 | 35 | 17 | 2.5 | 100 | 10.8 |
| Example 4-20 | R-20 | 105 | 33 | 19 | 2.4 | 110 | 10.9 |
| Example 4-21 | R-21 | 100 | 33 | 17 | 2.5 | 120 | 11.1 |

TABLE 6

| | | PEB | Optimum | | | | Collapse |
|---|---|---|---|---|---|---|---|
| | Resist | temperature | exposure dose | EL | LWR | DOF | limit |
| | composition | (° C.) | (mJ/cm²) | (%) | (nm) | (nm) | (nm) |
| Comparative | CR-1 | 95 | 39 | 13 | 3.2 | 90 | 12.9 |
| Example 4-1 | | | | | | | |
| Comparative | CR-2 | 100 | 37 | 14 | 3.3 | 80 | 12.8 |
| Example 4-2 | | | | | | | |
| Comparative | CR-3 | 100 | 38 | 14 | 4.2 | 80 | 13.1 |
| Example 4-3 | | | | | | | |
| Comparative | CR-4 | 100 | 38 | 14 | 3.1 | 90 | 12.9 |
| Example 4-4 | | | | | | | |
| Comparative | CR-5 | 95 | 40 | 14 | 3.5 | 80 | 12.3 |
| Example 4-5 | | | | | | | |
| Comparative | CR-6 | 100 | 39 | 15 | 2.9 | 90 | 12.1 |
| Example 4-6 | | | | | | | |
| Comparative | CR-7 | 100 | 39 | 13 | 2.9 | 90 | 12.3 |
| Example 4-7 | | | | | | | |
| Comparative | CR-8 | 100 | 38 | 14 | 3 | 80 | 12.1 |
| Example 4-8 | | | | | | | |
| Comparative | CR-9 | 95 | 39 | 14 | 3.1 | 90 | 12.7 |
| Example 4-9 | | | | | | | |
| Comparative | CR-10 | 100 | 37 | 14 | 3.2 | 80 | 12.3 |
| Example 4-10 | | | | | | | |
| Comparative | CR-11 | 100 | 39 | 14 | 2.8 | 80 | 12.1 |
| Example 4-11 | | | | | | | |

From the results shown in Tables 5 and 6, it was found that the chemically-amplified resist compositions containing a base polymer containing a repeating unit derived from the inventive onium-salt-type monomer had favorable sensitivity and excellent EL, LWR, and DOF. Furthermore, the collapse limit was small, and it was observed that the compositions were resistant to pattern collapse even in fine pattern formation. Thus, it was shown that the inventive chemically-amplified resist composition is suitable as a material for EUV lithography. On the other hand, in the Comparative Examples, where the inventive onium-salt-type monomer was not used, sensitivity, EL, LWR, DOF, and performance regarding pattern collapse were poor in every case.

### [5] EUV Lithography Evaluation (2)

### Examples 5-1 to 5-21 and Comparative Examples 5-1 to 5-11

Each of the chemically-amplified resist compositions (R-1 to R-21 and CR-1 to CR-11) shown in Tables 3 and 4 was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, the substrate was prebaked at 105° C. for 60 seconds using a hot plate to produce a resist film with 50 nm in film thickness. This resist film was exposed using an EUV scanner NXE3400 (NA 0.33, σ 0.9/0.6, quadrupole illumination, 46 nm in pitch on wafer size, hole pattern mask with +20% bias), manufactured by ASML Holding N.V. Then, PEB was performed at a temperature shown in Tables 7 and 8 for 60 seconds using a hot plate. Thereafter, development was performed with a 2.38 mass % aqueous TMAH solution for 30 seconds to form a hole pattern with 23 nm in size.

Using a length-measurement SEM (CG6300), manufactured by Hitachi High-Technologies Corporation, an exposure dose when the hole size was formed with 23 nm was measured to specify this exposure dose as sensitivity. Sizes of 50 holes were measured in this time, and a tripled value (3σ) of a standard variation (σ) calculated from the results was determined as size variation (CDU). Tables 7 and 8 show the results.

TABLE 7

| | | | Optimum | |
|---|---|---|---|---|
| | | PEB | exposure | |
| | Resist | temperature | dose | CDU |
| | composition | (° C.) | (mJ/cm²) | (nm) |
| Example 5-1 | R-1 | 95 | 22 | 2.3 |
| Example 5-2 | R-2 | 95 | 23 | 2.4 |
| Example 5-3 | R-3 | 90 | 23 | 2.3 |
| Example 5-4 | R-4 | 90 | 23 | 2.5 |
| Example 5-5 | R-5 | 90 | 22 | 2.3 |
| Example 5-6 | R-6 | 95 | 23 | 2.4 |
| Example 5-7 | R-7 | 95 | 24 | 2.2 |
| Example 5-8 | R-8 | 90 | 24 | 2.4 |
| Example 5-9 | R-9 | 95 | 23 | 2.5 |
| Example 5-10 | R-10 | 95 | 24 | 2.4 |
| Example 5-11 | R-11 | 95 | 25 | 2.3 |
| Example 5-12 | R-12 | 90 | 24 | 2.4 |
| Example 5-13 | R-13 | 90 | 23 | 2.4 |
| Example 5-14 | R-14 | 90 | 22 | 2.2 |
| Example 5-15 | R-15 | 90 | 24 | 2.3 |
| Example 5-16 | R-16 | 85 | 23 | 2.5 |
| Example 5-17 | R-17 | 95 | 23 | 2.3 |
| Example 5-18 | R-18 | 95 | 22 | 2.4 |
| Example 5-19 | R-19 | 90 | 24 | 2.4 |
| Example 5-20 | R-20 | 95 | 22 | 2.3 |
| Example 5-21 | R-21 | 95 | 24 | 2.4 |

TABLE 8

| | | | Optimum | |
|---|---|---|---|---|
| | | PEB | exposure | |
| | Resist | temperature | dose | CDU |
| | composition | (° C.) | (mJ/cm²) | (nm) |
| Comparative | CR-1 | 95 | 29 | 3.1 |
| Example 5-1 | | | | |
| Comparative | CR-2 | 95 | 30 | 2.8 |
| Example 5-2 | | | | |
| Comparative | CR-3 | 95 | 27 | 3.4 |
| Example 5-3 | | | | |
| Comparative | CR-4 | 90 | 27 | 2.9 |
| Example 5-4 | | | | |
| Comparative | CR-5 | 90 | 28 | 3 |
| Example 5-5 | | | | |
| Comparative | CR-6 | 95 | 27 | 2.9 |
| Example 5-6 | | | | |
| Comparative | CR-7 | 90 | 28 | 2.9 |
| Example 5-7 | | | | |
| Comparative | CR-8 | 90 | 29 | 3.1 |
| Example 5-8 | | | | |
| Comparative | CR-9 | 90 | 31 | 3.2 |
| Example 5-9 | | | | |
| Comparative | CR-10 | 95 | 30 | 3.4 |
| Example 5-10 | | | | |
| Comparative | CR-11 | 95 | 29 | 3.3 |
| Example 5-11 | | | | |

From the results shown in Tables 7 and 8, it was confirmed that the inventive chemically-amplified resist compositions had good sensitivity and excellent CDU. On the other hand, in the Comparative Examples, where the inventive onium-salt-type monomer was not used, sensitivity and CDU were poor in every case.

### [6] Dry Etching Resistance Evaluation

### Examples 6-1 to 6-21 and Comparative Examples 6-1 to 6-11

Each of the polymers (polymers P-1 to P-21 and comparative polymers CP-1 to CP-11) shown in Tables 1 and 2 was respectively dissolved in 10 g of cyclohexanone in an amount of 2 g. Each polymer solution was filtered with a 0.2-μm filter and applied to a Si substrate by spin-coating to form a film. The film was formed to have a thickness of 300 nm, and evaluation was carried out under the following conditions.

Etching Test with CHF₃/CF₄-Based Gas:

The difference between the film thicknesses of the polymer film before and after etching was determined using a dry etching apparatus TE-8500P manufactured by Tokyo Electron Limited.

The etching conditions are as shown below.

| | | |
|---|---|---|
| Chamber pressure | 40 | Pa |
| RF power | 1000 | W |
| Gap | 9 | mm |
| CHF₃ gas flow rate | 30 | mL/min |
| CF₄ gas flow rate | 30 | mL/min |
| Ar gas flow rate | 100 | mL/min |
| Time | 60 | sec |

In this evaluation, a smaller difference in film thickness, that is, a smaller amount reduced, indicates that the film has high etching resistance.

Tables 9 and 10 show the results of the dry etching resistance.

TABLE 9

| | | CHF₃/CF₄-based gas |
|---|---|---|
| | Polymer | etching rate (nm/min) |
| Example 6-1 | P-1 | 98 |
| Example 6-2 | P-2 | 97 |
| Example 6-3 | P-3 | 97 |
| Example 6-4 | P-4 | 98 |
| Example 6-5 | P-5 | 99 |
| Example 6-6 | P-6 | 97 |
| Example 6-7 | P-7 | 97 |
| Example 6-8 | P-8 | 98 |
| Example 6-9 | P-9 | 97 |
| Example 6-10 | P-10 | 96 |
| Example 6-11 | P-11 | 98 |
| Example 6-12 | P-12 | 98 |
| Example 6-13 | P-13 | 97 |
| Example 6-14 | P-14 | 96 |
| Example 6-15 | P-15 | 98 |
| Example 6-16 | P-16 | 97 |
| Example 6-17 | P-17 | 98 |
| Example 6-18 | P-18 | 97 |
| Example 6-19 | P-19 | 97 |
| Example 6-20 | P-20 | 98 |
| Example 6-21 | P-21 | 96 |

TABLE 10

| | | CHF₃/CF₄-based gas |
|---|---|---|
| | Polymer | etching rate (nm/min) |
| Comparative Example 6-1 | CP-1 | 114 |
| Comparative Example 6-2 | CP-2 | 102 |
| Comparative Example 6-3 | CP-3 | 103 |
| Comparative Example 6-4 | CP-4 | 100 |
| Comparative Example 6-5 | CP-5 | 112 |
| Comparative Example 6-6 | CP-6 | 109 |
| Comparative Example 6-7 | CP-7 | 102 |
| Comparative Example 6-8 | CP-8 | 100 |
| Comparative Example 6-9 | CP-9 | 104 |
| Comparative Example 6-10 | CP-10 | 103 |
| Comparative Example 6-11 | CP-11 | 103 |

From the results shown in Tables 9 and 10, it was confirmed that the inventive polymers had excellent dry etching resistance to CHF₃/CF₄-based gas. On the other hand, in the Comparative Examples, where the inventive onium-salt-type monomer was not used, dry etching resistance was poor in every case.

The present description includes the following inventions.
[1]: An onium-salt-type monomer represented by the following formula (a),

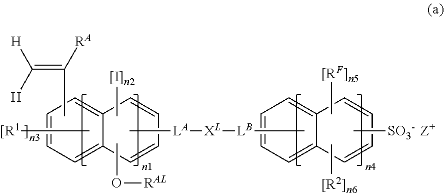

wherein “n1” represents 0 or 1, “n2” represents 1, 2, 3, or 4, “n3” represents 0, 1, or 2, provided that, when “n1” is 0, 1 n2+n3≤3 and when “n1” is 1, 1 n2+n3≤5, “n4” represents 0 or 1, “n5” represents 0, 1, 2, 3, or 4, and “n6” represents 0, 1, or 2, provided that when “n4” is 0, 0 n5+n6≤4 and when “n4” is 1, 0≤n5+n6≤6;
R^{A} represents a hydrogen atom, a fluorine atom, a methyl group, or a trifluoromethyl group;
R^{AL} represents an acid-labile group;
R¹ represents a halogen atom other than an iodine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “n3” is 2, the R's being identical to or different from each other, and the two R's optionally being bonded to each other to form a ring together with the carbon atoms bonded thereto;
R² represents a halogen atom other than a fluorine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “n6” is 2, the R²s being identical to or different from each other, and the two R²s optionally being bonded to each other to form a ring together with the carbon atoms bonded thereto;
R^{F} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms, when “n5” is 2, 3, or 4, the RFs being identical to or different from each other;
L^{A} and L^{B} each independently represent a single bond, an ether bond, an ester bond, a sulfonic acid ester bond, an amide bond, a sulfonic acid amide bond, a carbonate bond, or a carbamate bond;
X^{L} represents a single bond or a hydrocarbylene group having 1 to 40 carbon atoms and optionally containing a heteroatom; and
Z⁺ represents an onium cation.
[2]: The onium-salt-type monomer according to [1], represented by the following formula (a1),

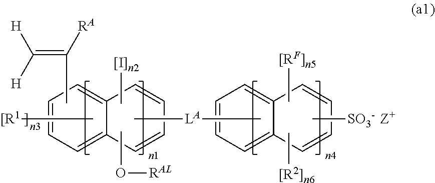

wherein “n1” to “n6”, R^{A}, R^{AL}, R¹, R², R^{F}, L^{A}, and Z⁺ are as defined above.
[3]: The onium-salt-type monomer according to [1] or [2], represented by the following formula (a2),

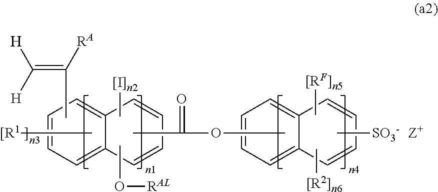

wherein “n1” to “n6”, R^{A}, R^{AL}, R¹, R², R^{F}, and Z⁺ are as defined above.
[4]: The onium-salt-type monomer according to any one of [1] to [3], wherein the acid-labile group represented by R^{AL} has a structure represented by the following formula (AL-1) or (AL-2),

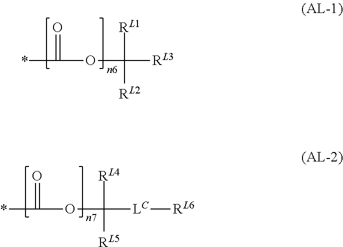

wherein “n6” represents 0 or 1 and “n7” represents 0 or 1;
R^{L1}, R^{L2}, and R^{L3} each independently represent a hydrocarbyl group having 1 to 12 carbon atoms, part of —CH₂— of the hydrocarbyl group optionally being substituted with —O— or —S—, when the hydrocarbyl group contains an aromatic ring, part or all of hydrogen atoms of the aromatic ring optionally being substituted with a halogen atom, a cyano group, a nitro group, an alkyl group having 1 to 4 carbon atoms and optionally containing a halogen atom, or an alkoxy group having 1 to 4 carbon atoms and optionally containing a halogen atom, and the R^{L1} and the R^{L2} optionally being bonded to each other to form a ring together with the carbon atom bonded thereto, part of —CH₂— of the ring optionally being substituted with —O— or —S—;
R^{L4} and R^{L5} each independently represent a hydrogen atom or a hydrocarbyl group having 1 to 10 carbon atoms and optionally containing a halogen atom, and R^{L6} represents a hydrocarbyl group having 1 to 20 carbon atoms and optionally containing a halogen atom, part of —CH₂— of the hydrocarbyl group optionally being substituted with —O— or —S—, and the R^{L5} and the R^{L6} optionally being bonded to each other to form a heterocyclic group having 3 to 20 carbon atoms together with the carbon atom and the L^{C} bonded thereto, part of the —CH₂— of the heterocyclic group optionally being substituted with —O— or —S—;
L^{C} represents —O— or —S—; and
“*” represents an attachment point to the adjacent —O—.
[5]: The onium-salt-type monomer according to any one of [1] to [4], wherein the onium cation represented by Z⁺ is a sulfonium cation represented by the following formula (Z-1) or an iodonium cation represented by the following formula (Z-2),

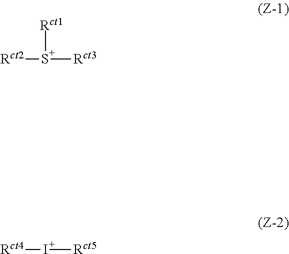

wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.
[6]: The onium-salt-type monomer according to any one of [1] to [4], wherein the onium cation represented by Z⁺ is a sulfonium cation represented by the following formula (Z-3),

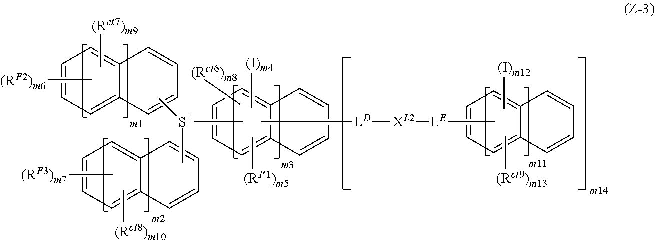

wherein “m1” represents 0 or 1, “m2” represents 0 or 1, “m3” represents 0 or 1, “m4” represents 0, 1, 2, 3, or 4, “m5” represents 0, 1, 2, 3, or 4, “m6” represents 0, 1, 2, 3, 4, 5, or 6, “m7” represents 0, 1, 2, 3, 4, 5, or 6, “m8” represents 0, 1, or 2, “m9” represents 0, 1, or 2, “m10” represents 0, 1, or 2, “m11” represents 0 or 1, “m12” represents 0, 1, 2, 3, or 4, “m13” represents 0, 1, or 2, and “m14” represents 0, 1, or 2, provided that, when “m1” is 0, 0 m6+m9≤4 and when “m1” is 1, 0 m6+m9≤6, when “m2” is 0, 0 m7+m10≤4 and when “m2” is 1, 0 m7+m10≤6, when “m3” is 0, 1 m4+m5+m8+m14≤4 and when “m3” is 1, 1 m4+m5+m8+m14≤6, when “m11” is 0, 0 m12+m13≤4 and when “m11” is 1, 0 m12+m13≤6, and m4+m12≤1;
R^{F1} to R^{F3} each independently represent a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms, when “m5” is 2 or more, the R^{F1}s being identical to or different from each other, when “m6” is 2 or more, the R^{F2}s being identical to or different from each other, and when “m7” is 2 or more, the R^{F3}s being identical to or different from each other;
R^{ct6} to R^{ct9} each represent a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m8” is 2, the two R^{ct6}s being identical to or different from each other and the two R^{ct6}s optionally being bonded to each other to form a ring together with the carbon atoms bonded thereto, when “m9” is 2, the two R^{ct7}s being identical to or different from each other and the two R^{ct7}s optionally being bonded to each other to form a ring together with the carbon atoms bonded thereto, when “m10” is 2, the two R^{ct3}s being identical to or different from each other and the two R^{ct3}s optionally being bonded to each other to form a ring together with the carbon atoms bonded thereto, and when “m13” is 2, the two R^{ct9}s being identical to or different from each other and the two R^{ct9}s optionally being bonded to each other to form a ring together with the carbon atoms bonded thereto;
the aromatic rings bonded directly to the S⁺ in the sulfonium cation are optionally bonded to each other to form a ring together with the S⁺;
L^{D} and L^{E} each independently represent a single bond, an ether bond, an ester bond, an amide bond, a sulfonic acid ester bond, a sulfonic acid amide bond, a carbonate bond, or a carbamate bond; and
X^{L2} represents a single bond or a hydrocarbylene group having 1 to 40 carbon atoms and optionally containing a heteroatom.
[7]: A monomeric photo-acid generator comprising the onium-salt-type monomer according to any one of [1] to [6].
[8]: A polymer comprising a repeating unit which is a derivative of the onium-salt-type monomer according to any one of [1] to [6].
[9]: The polymer according to [8], further comprising a repeating unit represented by the following formula (b1) or (b2),

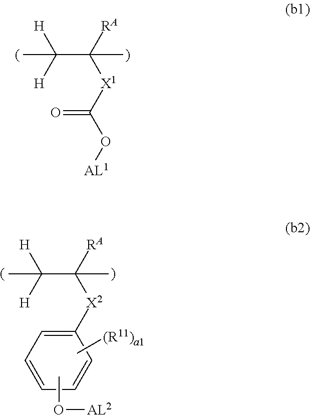

wherein each R^{A} independently represents a hydrogen atom, a fluorine atom, a methyl group, or a trifluoromethyl group;
X¹ represents a single bond, a phenylene group, a naphthylene group, *—C(═O)—O—X¹¹—, or *—C(═O)—NH—X¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, and X¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring;
X² represents a single bond, *—C(═O)—O—, or *—C(═O)—NH—;
“*” represents an attachment point to the carbon atom of the main chain;
R¹¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom;
AL¹ and AL² each independently represent an acid-labile group; and
“a1” represents 0, 1, 2, 3, or 4.
[10]: The polymer according to [8] or [9], further comprising a repeating unit represented by the following formula (b3),

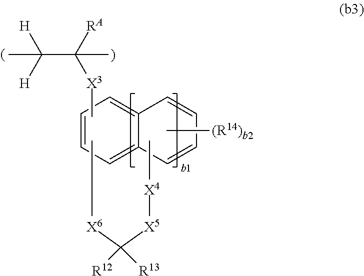

wherein “b1” represents 0 or 1, and “b2” represents 0, 1, 2, or 3 when “b1” is 0 and represents 0, 1, 2, 3, 4, or 5 when “b1” is 1;
R^{A} represents a hydrogen atom, a fluorine atom, a methyl group, or a trifluoromethyl group;
X³ represents a single bond, *—C(═O)—O—, or *—C(═O)—NH—, and “*” represents an attachment point to the carbon atom of the main chain;
R¹² and R¹³ each independently represent a hydrogen atom or a hydrocarbyl group having 1 to 20 carbon atoms and optionally containing a heteroatom, the R¹² and the R¹³ optionally being bonded to each other to form a ring together with the carbon atom bonded thereto;
R¹⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A})(R^{14B}), and R^{14A} and R^{14B} each independently represent a hydrogen atom or a hydrocarbyl group having 1 to 6 carbon atoms, when “b2” is 2 or more, the R¹⁴s optionally being bonded to each other to form a ring together with the carbon atoms of the aromatic ring bonded thereto;
X⁴ represents a single bond, an aliphatic hydrocarbylene group having 1 to 4 carbon atoms, a carbonyl group, a sulfonyl group, or a group which is a combination of the groups; and
X⁵ and X⁶ each independently represent an oxygen atom or a sulfur atom, provided that the X⁴ and the X⁶ are bonded to adjacent carbon atoms of the aromatic ring.
[11]: The polymer according to any one of [8] to [10], further comprising a repeating unit represented by the following formula (c),

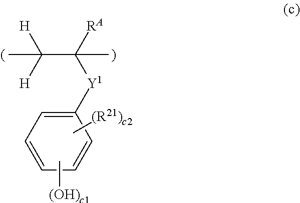

wherein R^{A} represents a hydrogen atom, a fluorine atom, a methyl group, or a trifluoromethyl group;
Y¹ represents a single bond, *—C(═O)—O—, or *—C(═O)—NH—, and “*” represents an attachment point to the carbon atom of the main chain;
R²¹ represents a halogen atom, a nitro group, a cyano group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
“c1” represents 1, 2, 3, or 4 and “c2” represents 0, 1, 2, or 3, provided that 1≤c1+c2≤5.
[12]: The polymer according to any one of [8] to [11], further comprising a repeating unit represented by the following formula (d),

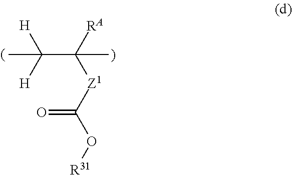

wherein R^{A} represents a hydrogen atom, a fluorine atom, a methyl group, or a trifluoromethyl group;
Z¹ represents a single bond, a phenylene group, a naphthylene group, *—C(═O)—O—Z¹¹—, or *—C(═O)—NH—Z¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, “*” represents an attachment point to the carbon atom of the main chain, and Z¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; and
R³¹ represents a hydrogen atom or a group having 1 to 20 carbon atoms and containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—).
[13]: A chemically-amplified resist composition comprising (A) a base polymer comprising the polymer according to any one of [8] to [12].
[14]: The chemically-amplified resist composition according to [13], further comprising one or more selected from (B) an organic solvent, (C) a quencher, (D) an acid generator, (E) a surfactant, and (F) a dissolution inhibitor.
[15]: A patterning process comprising the steps of:
   forming a resist film by using the chemically-amplified resist composition according to [13] or [14] on a substrate;
   exposing the resist film by using a high-energy beam; and
   developing the exposed resist film by using a developer.
[16]: The patterning process according to [15], wherein the high-energy beam is an ArF excimer laser beam having a wavelength of 193 nm, a KrF excimer laser beam having a wavelength of 248 nm, an electron beam, or an extreme ultraviolet ray having a wavelength of 3 to 15 nm.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. An onium-salt-type monomer represented by the following formula (a), 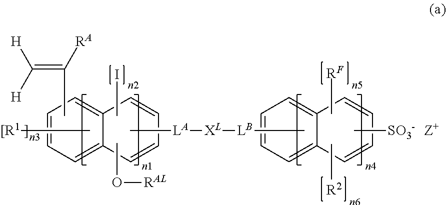
wherein “n1” represents 0 or 1, “n2” represents 1, 2, 3, or 4, “n3” represents 0, 1, or 2, provided that, when “n1” is 0, 1 n2+n3≤3 and when “n1” is 1, 1 n2+n3≤5, “n4” represents 0 or 1, “n5” represents 0, 1, 2, 3, or 4, and “n6” represents 0, 1, or 2, provided that when “n4” is 0, 0 n5+n6≤4 and when “n4” is 1, 0≤n5+n6≤6;
R^{A} represents a hydrogen atom, a fluorine atom, a methyl group, or a trifluoromethyl group;
R^{AL} represents an acid-labile group;
R¹ represents a halogen atom other than an iodine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “n3” is 2, the R's being identical to or different from each other, and the two R's optionally being bonded to each other to form a ring together with the carbon atoms bonded thereto;
R² represents a halogen atom other than a fluorine atom, a nitro group, a cyano group, a hydroxy group, an alkoxycarbonyl group, a carboxy group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “n6” is 2, the R²s being identical to or different from each other, and the two R²s optionally being bonded to each other to form a ring together with the carbon atoms bonded thereto;
R^{F} represents a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms, when “n5” is 2, 3, or 4, the R^{F}s being identical to or different from each other;
L^{A} and L^{B} each independently represent a single bond, an ether bond, an ester bond, a sulfonic acid ester bond, an amide bond, a sulfonic acid amide bond, a carbonate bond, or a carbamate bond;
X^{L} represents a single bond or a hydrocarbylene group having 1 to 40 carbon atoms and optionally containing a heteroatom; and
Z⁺ represents an onium cation.

2. The onium-salt-type monomer according to claim 1, represented by the following formula (a1), 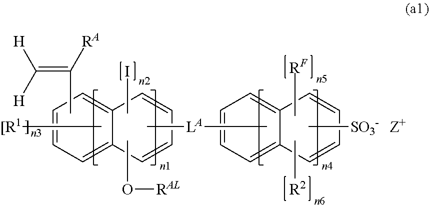
wherein “n1” to “n6”, R^{A}, R^{AL}, R¹, R², R^{F}, L^{A}, and Z⁺ are as defined above.

3. The onium-salt-type monomer according to claim 2, represented by the following formula (a2), 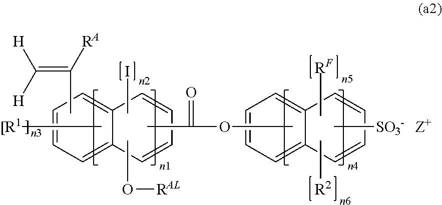
wherein “n1” to “n6”, R^{A}, R^{AL}, R¹, R², R^{F}, and Z⁺ are as defined above.

4. The onium-salt-type monomer according to claim 1, wherein the acid-labile group represented by R^{AL} has a structure represented by the following formula (AL-1) or (AL-2), 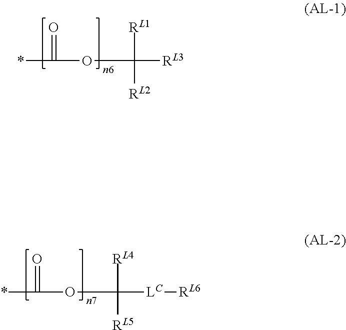
wherein “n6” represents 0 or 1 and “n7” represents 0 or 1;
R^{L1}, R^{L2}, and R^{L3} each independently represent a hydrocarbyl group having 1 to 12 carbon atoms, part of —CH₂— of the hydrocarbyl group optionally being substituted with —O— or —S—, when the hydrocarbyl group contains an aromatic ring, part or all of hydrogen atoms of the aromatic ring optionally being substituted with a halogen atom, a cyano group, a nitro group, an alkyl group having 1 to 4 carbon atoms and optionally containing a halogen atom, or an alkoxy group having 1 to 4 carbon atoms and optionally containing a halogen atom, and the R^{L1} and the R^{L2} optionally being bonded to each other to form a ring together with the carbon atom bonded thereto, part of —CH₂— of the ring optionally being substituted with —O— or —S—;
R^{L4} and R^{L5} each independently represent a hydrogen atom or a hydrocarbyl group having 1 to 10 carbon atoms and optionally containing a halogen atom, and R^{L6} represents a hydrocarbyl group having 1 to 20 carbon atoms and optionally containing a halogen atom, part of —CH₂— of the hydrocarbyl group optionally being substituted with —O— or —S—, and the R^{L5} and the R^{L6} optionally being bonded to each other to form a heterocyclic group having 3 to 20 carbon atoms together with the carbon atom and the L^{C} bonded thereto, part of the —CH₂— of the heterocyclic group optionally being substituted with —O— or —S—;
L^{C} represents —O— or —S—; and
“*” represents an attachment point to the adjacent —O—.

5. The onium-salt-type monomer according to claim 1, wherein the onium cation represented by Z⁺ is a sulfonium cation represented by the following formula (Z-1) or an iodonium cation represented by the following formula (Z-2), 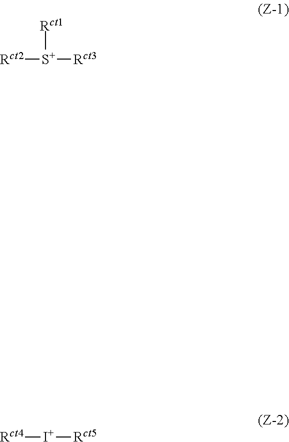
wherein R^{ct1} to R^{ct5} each independently represent a halogen atom or a hydrocarbyl group having 1 to 30 carbon atoms and optionally containing a heteroatom, the R^{ct1} and the R^{ct2} optionally being bonded to each other to form a ring together with the sulfur atom bonded thereto.

6. The onium-salt-type monomer according to claim 1, wherein the onium cation represented by Z⁺ is a sulfonium cation represented by the following formula (Z-3), 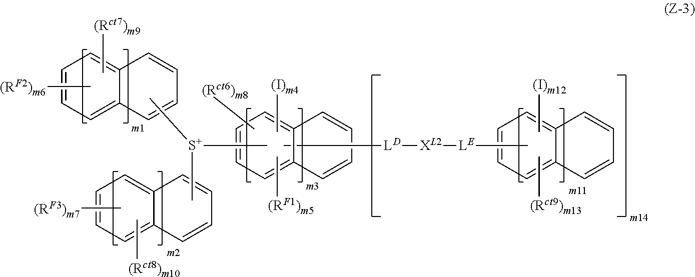
wherein “m1” represents 0 or 1, “m2” represents 0 or 1, “m3” represents 0 or 1, “m4” represents 0, 1, 2, 3, or 4, “m5” represents 0, 1, 2, 3, or 4, “m6” represents 0, 1, 2, 3, 4, 5, or 6, “m7” represents 0, 1, 2, 3, 4, 5, or 6, “m8” represents 0, 1, or 2, “m9” represents 0, 1, or 2, “m10” represents 0, 1, or 2, “m11” represents 0 or 1, “m12” represents 0, 1, 2, 3, or 4, “m13” represents 0, 1, or 2, and “m14” represents 0, 1, or 2, provided that, when “m1” is 0, 0 m6+m9≤4 and when “m1” is 1, 0 m6+m9≤6, when “m2” is 0, 0 m7+m10≤4 and when “m2” is 1, 0 m7+m10≤6, when “m3” is 0, 1 m4+m5+m8+m14≤4 and when “m3” is 1, 1 m4+m5+m8+m14≤6, when “m11” is 0, 0 m12+m13≤4 and when “m11” is 1, 0 m12+m13≤6, and m4+m12≤1;
R^{F1} to R^{F3} each independently represent a fluorine atom, a fluorinated saturated hydrocarbyl group having 1 to 6 carbon atoms, a fluorinated saturated hydrocarbyloxy group having 1 to 6 carbon atoms, or a fluorinated saturated hydrocarbylthio group having 1 to 6 carbon atoms, when “m5” is 2 or more, the R^{F1}s being identical to or different from each other, when “m6” is 2 or more, the R^{F2}s being identical to or different from each other, and when “m7” is 2 or more, the R^{F3}s being identical to or different from each other;
R^{ct6} to R^{ct9} each represent a halogen atom other than an iodine atom and a fluorine atom, a nitro group, a cyano group, a hydrocarbyl group having 1 to 20 carbon atoms and optionally containing a heteroatom, a hydrocarbyloxy group having 1 to 20 carbon atoms and optionally containing a heteroatom, or a hydrocarbylthio group having 1 to 20 carbon atoms and optionally containing a heteroatom, when “m8” is 2, the two R^{ct6}s being identical to or different from each other and the two R^{ct6}s optionally being bonded to each other to form a ring together with the carbon atoms bonded thereto, when “m9” is 2, the two R^{ct7}s being identical to or different from each other and the two R^{ct7}s optionally being bonded to each other to form a ring together with the carbon atoms bonded thereto, when “m10” is 2, the two R^{ct3}s being identical to or different from each other and the two R^{ct3}s optionally being bonded to each other to form a ring together with the carbon atoms bonded thereto, and when “m13” is 2, the two R^{ct9}s being identical to or different from each other and the two R^{ct9}s optionally being bonded to each other to form a ring together with the carbon atoms bonded thereto;
the aromatic rings bonded directly to the S⁺ in the sulfonium cation are optionally bonded to each other to form a ring together with the S⁺;
L^{D} and L^{E} each independently represent a single bond, an ether bond, an ester bond, an amide bond, a sulfonic acid ester bond, a sulfonic acid amide bond, a carbonate bond, or a carbamate bond; and
X^{L2} represents a single bond or a hydrocarbylene group having 1 to 40 carbon atoms and optionally containing a heteroatom.

7. A monomeric photo-acid generator comprising the onium-salt-type monomer according to claim 1.

8. A polymer comprising a repeating unit which is a derivative of the onium-salt-type monomer according to claim 1.

9. A polymer comprising a repeating unit which is a derivative of the onium-salt-type monomer according to claim 2.

10. A polymer comprising a repeating unit which is a derivative of the onium-salt-type monomer according to claim 3.

11. A polymer comprising a repeating unit which is a derivative of the onium-salt-type monomer according to claim 4.

12. A polymer comprising a repeating unit which is a derivative of the onium-salt-type monomer according to claim 5.

13. The polymer according to claim 8, further comprising a repeating unit represented by the following formula (b1) or (b2), 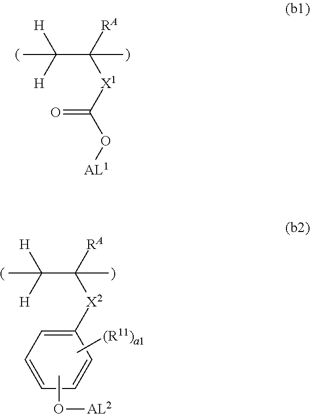
wherein each R^{A} independently represents a hydrogen atom, a fluorine atom, a methyl group, or a trifluoromethyl group;
X¹ represents a single bond, a phenylene group, a naphthylene group, *—C(═O)—O—X¹¹—, or *—C(═O)—NH—X¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, and X¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring;
X² represents a single bond, *—C(═O)—O—, or *—C(═O)—NH—;
“*” represents an attachment point to the carbon atom of the main chain;
R¹¹ represents a halogen atom, a cyano group, a hydroxy group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom;
AL¹ and AL² each independently represent an acid-labile group; and
“a1” represents 0, 1, 2, 3, or 4.

14. The polymer according to claim 8, further comprising a repeating unit represented by the following formula (b3), 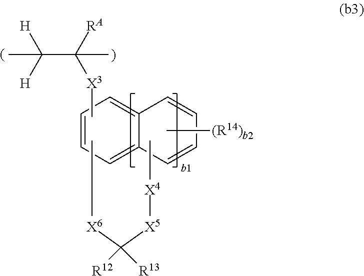
wherein “b1” represents 0 or 1, and “b2” represents 0, 1, 2, or 3 when “b1” is 0 and represents 0, 1, 2, 3, 4, or 5 when “b1” is 1;
R^{A} represents a hydrogen atom, a fluorine atom, a methyl group, or a trifluoromethyl group;
X³ represents a single bond, *—C(═O)—O—, or *—C(═O)—NH—, and “*” represents an attachment point to the carbon atom of the main chain;
R¹² and R¹³ each independently represent a hydrogen atom or a hydrocarbyl group having 1 to 20 carbon atoms and optionally containing a heteroatom, the R¹² and the R¹³ optionally being bonded to each other to form a ring together with the carbon atom bonded thereto;
R¹⁴ represents a halogen atom, a hydroxy group, a cyano group, a nitro group, a hydrocarbyl group having 1 to 20 carbon atoms and optionally containing a heteroatom, a hydrocarbyloxy group having 1 to 20 carbon atoms and optionally containing a heteroatom, a hydrocarbyloxycarbonyl group having 2 to 20 carbon atoms and optionally containing a heteroatom, a hydrocarbylthio group having 1 to 20 carbon atoms and optionally containing a heteroatom, or —N(R^{14A})(R^{14B}), and R^{14A} and R^{14B} each independently represent a hydrogen atom or a hydrocarbyl group having 1 to 6 carbon atoms, when “b2” is 2 or more, the R¹⁴s optionally being bonded to each other to form a ring together with the carbon atoms of the aromatic ring bonded thereto;
X⁴ represents a single bond, an aliphatic hydrocarbylene group having 1 to 4 carbon atoms, a carbonyl group, a sulfonyl group, or a group which is a combination of the groups; and
X⁵ and X⁶ each independently represent an oxygen atom or a sulfur atom, provided that the X⁴ and the X⁶ are bonded to adjacent carbon atoms of the aromatic ring.

15. The polymer according to claim 8, further comprising a repeating unit represented by the following formula (c), 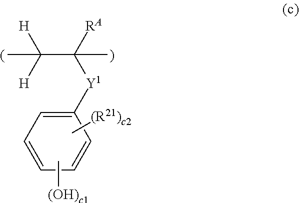
wherein R^{A} represents a hydrogen atom, a fluorine atom, a methyl group, or a trifluoromethyl group;
Y¹ represents a single bond, *—C(═O)—O—, or *—C(═O)—NH—, and “*” represents an attachment point to the carbon atom of the main chain;
R²¹ represents a halogen atom, a nitro group, a cyano group, a carboxy group, a hydrocarbyl group having 1 to 20 carbon atoms and optionally containing a heteroatom, a hydrocarbyloxy group having 1 to 20 carbon atoms and optionally containing a heteroatom, a hydrocarbylcarbonyl group having 2 to 20 carbon atoms and optionally containing a heteroatom, a hydrocarbylcarbonyloxy group having 2 to 20 carbon atoms and optionally containing a heteroatom, or a hydrocarbyloxycarbonyl group having 2 to 20 carbon atoms and optionally containing a heteroatom; and
“c1” represents 1, 2, 3, or 4 and “c2” represents 0, 1, 2, or 3, provided that 1≤c1+c2≤5.

16. The polymer according to claim 8, further comprising a repeating unit represented by the following formula (d), 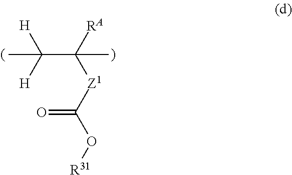
wherein R^{A} represents a hydrogen atom, a fluorine atom, a methyl group, or a trifluoromethyl group;
Z¹ represents a single bond, a phenylene group, a naphthylene group, *—C(═O)—O—Z¹¹—, or *—C(═O)—NH—Z¹¹—, the phenylene group or naphthylene group optionally being substituted with a hydroxy group, a nitro group, a cyano group, a saturated hydrocarbyl group having 1 to 10 carbon atoms and optionally containing a fluorine atom, a saturated hydrocarbyloxy group having 1 to 10 carbon atoms and optionally containing a fluorine atom, or a halogen atom, “*” represents an attachment point to the carbon atom of the main chain, and Z¹¹ represents a saturated hydrocarbylene group having 1 to 10 carbon atoms, a phenylene group, or a naphthylene group, the saturated hydrocarbylene group optionally containing a hydroxy group, an ether bond, an ester bond, or a lactone ring; and
R³¹ represents a hydrogen atom or a group having 1 to 20 carbon atoms and containing at least one structure selected from a hydroxy group other than a phenolic hydroxy group, a cyano group, a carbonyl group, a carboxy group, an ether bond, an ester bond, a sulfonic acid ester bond, a carbonate bond, a lactone ring, a sultone ring, and a carboxylic anhydride (—C(═O)—O—C(═O)—).

17. A chemically-amplified resist composition comprising (A) a base polymer comprising the polymer according to claim 8.

18. The chemically-amplified resist composition according to claim 17, further comprising one or more selected from (B) an organic solvent, (C) a quencher, (D) an acid generator, (E) a surfactant, and (F) a dissolution inhibitor.

19. A patterning process comprising the steps of:
forming a resist film by using the chemically-amplified resist composition according to claim 17 on a substrate;
exposing the resist film by using a high-energy beam; and
developing the exposed resist film by using a developer.

20. The patterning process according to claim 19, wherein the high-energy beam is an ArF excimer laser beam having a wavelength of 193 nm, a KrF excimer laser beam having a wavelength of 248 nm, an electron beam, or an extreme ultraviolet ray having a wavelength of 3 to 15 nm.
